(19) United States
(12) Patent Application Publication
(10) Pub. No.: US 2018/0362485 A1
(43) Pub. Date: Dec. 20, 2018

(54) N-HYDROXYFORMAMIDE COMPOUNDS AND COMPOSITIONS COMPRISING THEM FOR USE AS BMP l, TLL1 AND/OR TLL2 INHIBITORS

(71) Applicant: GlaxoSmithKline Intellectual Property (No. 2) Limited, Brentford, Middlesex (GB)

(72) Inventors: Carla A. DONATELLI, King of Prussia, PA (US); Sarah E. DOWDELL, King of Prussia, PA (US); Mark ELBAN, King of Prussia, PA (US); Mark A. HILFIKER, King of Prussia, PA (US); Tram H. HOANG, King of Prussia, PA (US); Dennis Alan HOLT, King of Prussia, PA (US); Sharada MANNS, King of Prussia, PA (US); Andrew MARCUS, King of Prussia, PA (US); Craig POTTEIGER, King of Prussia, PA (US); Raynold SHENJE, King of Prussia, PA (US); David G. WASHBURN, King of Prussia, PA (US)

(21) Appl. No.: 15/736,911

(22) PCT Filed: Jul. 8, 2016
(86) PCT No.: PCT/IB2016/054123

§ 371 Date: Dec. 15, 2017

Related U.S. Application Data
(60) Provisional application No. 62/190,345, filed on Jul. 9, 2015.

(51) Int. Cl. C07D 307/68; C07F 9/655; C07D 405/12; C07D 413/12; C07F 9/6558

(52) U.S. Cl. CPC C07D 307/68; C07F 9/65515; C07D 405/12; C07F 9/65522; C07F 9/65586; C07F 9/65583; C07D 413/12

(57) ABSTRACT

Compounds of Formulas (I) and (II):

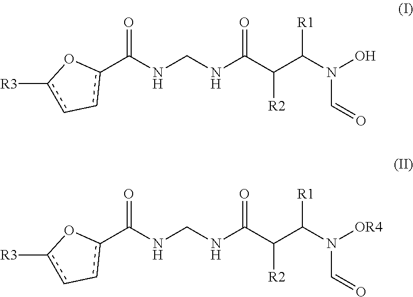

and salts thereof; methods of making and using the same, including use for inhibiting BMP1, TLL1 and/or TLL2 and in treatment of diseases associated with BMP1, TLL1 and/or TLL2 activity.

### FIELD OF THE INVENTION

The present invention relates to compounds and/or compositions thereof that inhibit BMP1 (also known as BMP-1, bone morphogenic protein 1, bone morphogenetic protein 1, procollagen C-proteinase, and procollagen C-endopeptidase), Tolloid-like 1 (TLL1) and/or Tolloid-like 2 (TLL2) metalloproteases, inclusive of isoforms, in particular multiple isoforms encoded by RNA splice variants, and methods of making and using the same.

### BACKGROUND OF THE INVENTION

Fibrous collagens are integral parts of the extracellular matrix that support tissue integrity and maintain the cellular microenvironment for normal physiological functions. Collagens I-III, the major isoforms of the fibrous collagen protein family, are synthesized as procollagen precursors containing N-terminal and C-terminal propeptides. The procollagens are post-translationally modified by proline hydroxylation, and secreted into the peri-vascular space for further processing. N-terminal propeptides of the collagens are subsequently cleaved by proteinases of the ADAMTS (A Distintegrin And Metalloproteinase with ThromboSpondin repeats) family, while the C-terminal propeptides are processed by the Tolloid family of metalloproteases, which include BMP1, TLL1 and TLL2 (Hopkins, D. R. et al., Matrix Biology, 2007, 26, 508-523). The cleavage of both N-terminal and C-terminal propeptides allows further maturation of the collagen, leading to cross-linking at lysine residues and formation of insoluble fibrillar structures (Shoulders, M. D. et al., Annual Review of Biochemistry, 2009, 78, 929-958).

Whereas the BMP1, TLL1 and TLL2 proteins are encoded by separate genes, this family also includes isoforms of BMP1, including multiple isoforms of BMP1 that result from alternative splicing of the same gene product (see e.g., Takahara, K., et al., The Journal of Biological Chemistry, 1994, 269. 32572-32578; and Cvetjeticanin, B. et al., Medical Hypotheses, 2014, 83, 656-658). The originally discovered form of BMP1 is designated BMP-1-1 or BMP1-1. Other BMP1 isoforms encoded by splice variant RNA transcripts have been described at the transcriptional level and designated with sequential suffixes, e.g., as BMP-1-2, BMP-1-3, BMP-1-4, BMP-1-5, BMP-1-6, and BMP-1-7 (see, e.g., Wozney et al., Science (1988), 242: 1528-1534; Kessler et al., Science, (1996) 271: 360-362; Li et al., Proc. Natl. Acad. Sci. USA (1996), 93: 5127-5130; Janitz et al., J. Mol. Med. (1998), 76: 141-146; Takahara et al., J. Biol. Chem. (1994), 269: 32572-32578; and Ge and Greenspan, Birth Defect Res. (2006), 78: 47-68).

A number of BMP1 isoforms have also been confirmed at the protein level as circulating in the blood of patients with various diseases and in healthy humans (see, e.g., International Patent publication Nos. WO2008/011193 A2 and WO2013/163479 A1, and Grgurevic et al., J. Am. Soc. Nephrol. (2011), 21:681-692). In addition, the role of BMP1 in processing procollagen leading to fibrosis and scar tissue formation in a variety of diseases as well as the discovery of blood profiles comprising individual BMP1 isoforms in patients with various diseases has made BMP1 an attractive target for developing new therapies (see, e.g. WO2008/011193 A2; WO2013/163479 A1; Grgurevic et al., J. Am. Soc. Nephrol. (2011), 21:681-692, Cvetjeticanin, B. et al., Medical Hypotheses, 2014, 83, 656-658; and Turtle et al., Expert Opin. Ther. Patents (2004), 14(8):1185-1197).

For TLL1, up to six theoretical RNA splice variants (www.ensembl.org; gene identifier ENSG00000038295) may exist and at least two expressed proteins (www.uniprot.org, protein identifier 043897) have been reported. Three RNA splice variants have been reported for the gene that encodes TLL2 (www.ensembl.org; gene identifier ENSG00000095587), one of which is thought to encode functional protein (www.uniprot.org, protein identifier Q9Y6L7).

Excessive production of extracellular matrix (ECM) proteins, including collagen, can lead to fibrotic pathologies in various organs or tissues that may be associated with increased tissue rigidity, parenchymal replacement, aberrant electrical conductance, sclerotic wound healing (e.g. infarction and burns), and/or abnormal cell-cell interactions. For example, increased fibrosis and collagen production are consistently observed in patients with acute and chronic cardiac diseases, e.g., heart failure, arrhythmias, hypertrophic cardiomyopathy, and myocardial infarction (Lopez, B. et al., Circulation, 2010, 121, 1645-1654; Ho, C. Y., et al., New England Journal of Medicine, 2010, 363, 552-563; Kostin, S. et al., Cardiovascular Research, 2002, 54, 361-379; See, F., et al., Current Pharmaceutical Design, 2005, 11, 477-487; Cvetjeticanin, B. et al. Medical Hypotheses, 2014, 83, 656-658), chronic obstructive pulmonary disease (“COPD”) (Salazar, L. M., et al., Lung, 2011, 189, 101-109), liver cirrhosis and nonalcoholic steatohepatitis (“NASH”) (Bataller, R., et al., Journal of Clinical Investigation, 2005, 115, 209-218), idiopathic pulmonary fibrosis (Chakraborty, S, et al., Expert Opin Investig Drugs, 2014, 23, 893-910), collagen vascular diseases, e.g. systemic lupus erythematosus, rheumatoid arthritis and scleroderma (Eckes, B., et al., J Mol Med, 2014, 92, 913-924), muscular dystrophies (e.g., Serrano, A. C., et al., Experimental Cell Research, 2010, 316, 3050-3058; Klingler, W., et al., Acta Myoligica, XXXI, 2012, 184-195), chronic kidney disease (Liu, Y., Nature Reviews Nephrology, 2011, 7, 684-696), acute kidney injury (Molitoris, B., The Journal of clinical Investigation, 2014, 124, 2355-2363; Venkatachalam, M. A. et al., Am J Physiol Renal Physiol 298: F1078-F1094, 2010), diabetic nephropathy (Sun, Y. M., et a., Biochemical and Biophysical Research Communications, 2013, 433, 359-361), keloids, wound healing, adhesions, hypertrophic and other scarring associated with, e.g. burns, surgery and other trauma (Meier K., et al., Expert Opinion on Emerging Drugs, 2006, 11, 39-47; Malecaze, F., et al., Investigative Opthalmology and Visual Science, 2014, 55, 6712-6721; van der Weer, W. et al., Burns, 2009, 35, 15-29), stroke, multiple sclerosis and spinal cord injury (Fernandez-Klett, F. and Piller, J. Brain Pathology, 2014, 24, 404-13; Rimar, D. et al., Arthritis & Rheumatology, Vol. 66, No. 3, March 2014, 726-730). Therefore, reducing excessive collagen production and maturation by targeting the BMP1, TLL1 and/or TLL2 pathway(s) can be an effective therapeutic strategy for treating fibrotic pathologies such as these diseases. This is supported by recent published studies using pharmacological agents that inhibit BMP1, TLL1 and/or TLL2 activity in cardiac and kidney disease models in small animals (Grgurevic, L., et al., Journal of the AmericanSociety of Nephrology, 2011, 21, 681-692; He, W., et al., Proceedings of the National Academy of Sciences, 2010, 107, 21110-21115; Cvetjeticanin, B. et al., Medical Hypotheses, 2014, 83, 656-658; International Patent publication Nos. WO2008/011193 A2 and WO2013/163479 A1).

The Tolloid family of metalloproteases (BMP1, TLL1 and TLL2) has additional substrates beyond collagens that may also contribute to its role in promoting ECM protein production. For example, the pro-form of lysyl oxidase 1 (LOX1) has been shown to be a substrate of BMP1, and cleavage by BMP1 enhances the LOX enzyme activity and thereby induces collagen cross-linking (Uzel, M. I., et al., Journal of Biological Chemistry, 2001, 276, 22537-22543). Thus, BMP1 also has a role in the development of pathological tissue stiffness via this mechanism, for example in glaucoma (Tovar-Vidales, T., et al., Investigative Ophthalmology & Visual Science, 2013, 54, 4741-4748) and in diastolic dysfunction in the heart (López, B., et al., American Journal of Physiology—Heart and Circulatory Physiology, 2010, 299, H1-H9). BMP1 also cleaves additional collagens, e.g. procollagens V and XII which influence fibril size and shape, as well as non-fibrillar procollagen VII (Hopkins, D. R. et al., Matrix Biology, 2007, 26, 508-523). Latent TGF-beta binding protein (LTBP) has also been shown to be cleaved by BMP1, allowing enhanced TGF-beta action to induce further collagen production (Ge, G., et al., Journal of Cell Biology, 2006, 175, 111-120). Regulation of TGF-beta by BMP1 may also play roles in other pathologies, such as control of cancer cell metastasis and invasion (Wu, X., et al. Oncogene, 2014, 33, 1506-1514). Similarly, BMP1, TLL1 and/or TLL2 also activate a broader range of other TGF-beta like molecules, such as BMPs 2 and 4, by proteolytically processing interacting proteins (Hopkins, D. R. et al., Matrix Biology, 2007, 26, 508-523). The combined actions of BMP1 and its various substrates suggest that BMP1, TLL1 and TLL2 are key regulators of tissue ECM production/maturation and that the members of the tolloid family of metalloproteases are particularly effective targets for anti-fibrosis therapeutic intervention.

BMP1, TLL1 and TLL2 may also affect other biological pathways via additional substrate processing. In particular, they may affect muscle biology via promoting activation of myostatin. Myostatin is a hormone that negatively regulates muscle growth (Lee, S. J., 2004, Annual Review of Cell & Developmental Biology, 20, 61-86). BMP1 has been demonstrated to cleave an inhibitory pro-peptide of myostatin and thus enhance myostatin activity (Wolfman N. M., et al., Proceedings of the National Academy of Sciences, 2003, 100, 15842-15846). Knockout of TLL2 in mice demonstrated enhanced muscle mass, thereby providing support for the connection between tolloid metalloprotease and myostatin (Lee, S. J., PLoS one, 2008, 3, e1628). An inhibitor of BMP1, TLL1 and/or TLL2 could therefore be beneficial in diseases where muscle function or muscle mass is diminished, including muscular dystrophy, sarcopenia, and cachexia associated with, e.g., heart failure, CKD, COPD, cancer or old age.

Taken together, the biology of BMP1, TLL1 and TLL2 lends strong support for their key roles in collagen processing, assembly and cross-linking, leading to the formation of a fibrillar collagen network that maintains tissue integrity and proper cellular microenvironment. This family of proteins may also play important roles in the etiology of fibrotic conditions, for example in the heart, lung, skeletal muscle, kidney, liver, skin, vasculature, nervous system, and eye, and inhibitors of these metalloproteases may provide broad benefits as anti-fibrotic agents for the treatment of diseases associated with fibrosis, such as myocardial infarction, heart failure, cardiac arrhythmias, hypertrophic cardiomyopathy, chronic kidney disease (CKD), post-acute kidney injury, diabetic nephropathy, delayed graft function post-transplantation, chronic obstructive pulmonary disease (COPD), idiopathic pulmonary fibrosis (IPF), liver cirrhosis, non-alcoholic steatohepatitis (NASH), muscular dystrophies (e.g., Duchenne, Becker, limb-girdle, congenital, facioscapulohumeral, myotonic, oculopharyngeal, distal, and Emery-Dreifuss), glaucoma, corneal scarring, keloids, wound healing, adhesions, hypertrophic scarring, other scarring, e.g. associated with burns, surgery or other trauma, stroke, collagen vascular diseases such as systemic lupus erythematosus, rheumatoid arthritis and scleroderma, spinal cord injury and multiple sclerosis. Furthermore, BMP1, TLL1 and TLL2 inhibitors may have additional therapeutic applications in muscular disease based on their impact on myostatin biology, in particular muscular dystrophies (e.g., Duchenne, Becker, limb-girdle, congenital, facioscapulohumeral, myotonic, oculopharyngeal, distal, and Emery-Dreifuss), sarcopenia, and cachexia associated with, e.g., heart failure, CKD, COPD, cancer or old age.

Small molecule BMP1, TLL1 and/or TLL2 inhibitors have recently been discovered and described in PCT application no. PCT/IB2015/050179, filed Jan. 9, 2015, published Jul. 16, 2015 as PCT publication no. WO2015/104684, each incorporated herein by reference. For example, PCT application no. PCT/IB2015/050179 and WO2015/104684 disclose compounds of Formula I:

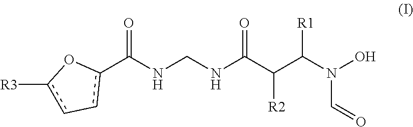

wherein:
R1 is selected from the group consisting of H, (C₁-C₄) straight chain alkyl, and (C₁-C₄) straight chain alkyl substituted with a hydroxy group;
R2 is selected from H, (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl, wherein heterocyclyl is a monocyclic ring having 5-6 ring atoms wherein 1-2 of the ring atoms are selected from nitrogen, oxygen and sulfur, and wherein said (C₁-C₁₁)alkyl, cycloalkyl, phenyl, naphthyl and heterocyclyl may be optionally substituted with 1-2 groups independently selected from (C₁-C₄)alkyl, (C₁-C₄)alkoxy, halo, and cyano; and
R3 is selected from:

a) phenyl, optionally substituted with 1-3 groups independently selected from:

(C₁-C₆)alkyl, optionally substituted with 1-3 groups independently selected from: fluoro (e.g., —CF₃); —CO₂H; —P(O)R^{f}R^{g}; NR^{a}R^{b} wherein R^{a} is selected from H and (C₁-C₄)alkyl and R^{b} is selected from (C₁-C₄)alkyl substituted with —CO₂H or —P(O)R^{f}R^{g}, and —C(O)NR^{a}R^{b} wherein R^{a} and R^{b} are independently selected from H and (C₁-C₄)alkyl, wherein the (C₁-C₄)alkyl is optionally substituted with 1-3 groups independently selected from hydroxy, —CO₂H, —C(O)O(C₁-C₄)alkyl and —P(O)R^{f}R^{g};
cyclopropyl, optionally substituted with 1 —CO₂H;
—C(O)NR^{a}R^{b} wherein R^{a} and R^{b} are independently selected from H and (C₁-C₄)alkyl, wherein the (C₁-C₄)alkyl is optionally substituted with 1-3 groups independently selected from hydroxy, —CO₂H, —C(O)O(C₁-C₄)alkyl, —P(O)R^{f}R^{g}, NR^{c}R^{d} and N⁺R^{c}R^{d}R^{e};
(C₁-C₆)alkoxy, optionally substituted with 1-3 substituents independently selected from halo, hydroxy, —CO₂H, (C₃-C₆)cycloalkyl, C(O)NH₂ and pyrrolidinyl;
(C₃-C₆)cycloalkoxy, optionally substituted with 1-3 substituents independently selected from halo, hydroxy, and —CO₂H;
—NR^{a}R^{b} wherein R^{a} and R^{b} are independently selected from H and (C₁-C₄)alkyl, wherein the (C₁-C₄)alkyl is optionally substituted with 1-3 groups independently selected from oxo and —CO₂H;
—SR^{a} wherein R^{a} is selected from H and (C₁-C₄)alkyl;
—CO₂H; —C(NOH)NH₂, cyano; —C(O)O(C₁-C₄)alkyl; —C(O)CO₂H; —P(O)R^{f}R^{g}; —OP(O)R^{f}R^{g}; halo; hydroxy; nitro; —NHSO₂(C₁-C₂)alkyl; —SO₃H; —SO₂(C₁-C₂)alkyl; —SO₂NR^{c}R^{d}; —SO₂NHC(O)(C₁-C₂)alkyl; and —B(OH)₂;

and

b) heteroaryl, optionally substituted with 1-2 groups independently selected from:

(C₁-C₄)alkyl, (C₁-C₄)alkoxy, oxo, —CO₂H, —P(O)R^{f}R^{g}, and —OP(O)R^{f}R^{g};
wherein in each occurrence: R^{c}, R^{d} and R^{e} are independently selected from H and (C₁-C₂)alkyl; and R^{f} and R^{g} are independently selected from hydroxy, (C₁-C₂)alkyl and (C₁-C₂)alkoxy;
and salts, particularly pharmaceutically acceptable salts, thereof.

Compounds disclosed therein were found to have inhibitory activity against BMP1, TLL1 and/or TLL2.

The present invention discloses novel compounds which are BMP1, TLL1 and/or TLL2 inhibitors and/or which reveal (convert to or generate) a BMP1, TLL1 and/or TLL2 inhibitor in vivo, ex vivo or in vitro. In some embodiments the compound is a derivative of a BMP1, TLL1 and/or TLL2 inhibitor disclosed in PCT application no. PCT/IB2015/050179 or PCT publication no. WO2015/104684. In some embodiments, the compound reveals a BMP1, TLL1 and/or TLL2 inhibitor disclosed in PCT application no. PCT/IB2015/050179 or PCT publication no. WO2015/104684.

### SUMMARY OF THE INVENTION

The present invention discloses novel compounds which are BMP1, TLL1 and/or TLL2 inhibitors and/or which reveal (convert to) a BMP1, TLL1 and/or TLL2 inhibitor in vivo, ex vivo or in vitro. In some embodiments the compound is a derivative of a BMP1, TLL1 and/or TLL2 inhibitor disclosed in PCT application no. PCT/IB2015/050179 or PCT publication no. WO2015/104684. In some embodiments, the compound reveals a BMP1, TLL1 and/or TLL2 inhibitor disclosed in PCT application no. PCT/IB2015/050179 or PCT publication no. WO2015/104684.

In one aspect, the present invention relates to compounds of Formula (I):

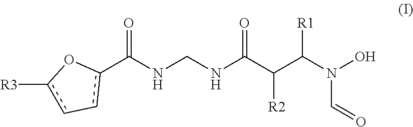

or a salt thereof,
wherein:
R1 is selected from the group consisting of H, (C₁-C₄) straight chain alkyl, and (C₁-C₄) straight chain alkyl substituted with one hydroxy group;
R2 is selected from H, (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl, wherein heterocyclyl is a monocyclic ring having 5-6 ring atoms wherein 1-2 of the ring atoms are selected from nitrogen, oxygen and sulfur, and wherein said (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl are optionally substituted with 1-2 groups independently selected from (C₁-C₄)alkyl, (C₁-C₄)alkoxy, halo, and cyano; and
R3 is selected from phenyl and heteroaryl, wherein said phenyl and heteroaryl are substituted with 1-3 substituents independently selected from:

(C₁-C₆)alkyl, optionally substituted with 1-3 groups independently selected from: fluoro; —CO₂R′; —P(O)R″R″; —NR^{a}R^{b} wherein R^{a} is selected from H and (C₁-C₄)alkyl and R^{b} is selected from (C₁-C₄)alkyl substituted with 1-3 groups independently selected from —CO₂R′ and —P(O)R″R″; and —C(O)NR^{a1}R^{b1} wherein R^{a1} and R^{b1} are independently selected from H and (C₁-C₄)alkyl, wherein the (C₁-C₄)alkyl is optionally substituted with 1-3 groups independently selected from hydroxy, —CO₂R′, -and —P(O)R″R″;

cyclopropyl, optionally substituted with one —CO₂R′;

—C(O)NR^{a2}R^{b2} wherein R^{a2} and R^{b2} are independently selected from H and (C₁-C₄)alkyl, wherein the (C₁-C₄)alkyl is optionally substituted with 1-3 groups independently selected from hydroxy, —CO₂R′, —P(O)R″R″, —NR^{c}R^{d} and —N⁺R^{c}R^{d}R^{e};

(C₁-C₆)alkoxy, optionally substituted with 1-3 substituents independently selected from halo, hydroxy, —CO₂R′, (C₃-C₆)cycloalkyl, —C(O)NH₂ and pyrrolidinyl;

(C₃-C₆)cycloalkoxy, optionally substituted with 1-3 substituents independently selected from halo, hydroxy, and —CO₂R′;

—NR^{a3}R^{b3} wherein R^{a3} and R^{b3} are independently selected from H and (C₁-C₄)alkyl, wherein the (C₁-C₄)alkyl is optionally substituted with 1-3 groups independently selected from oxo and —CO₂R′;

—SR^{a4} wherein R^{a4} is selected from H and (C₁-C₄)alkyl;

—CO₂R′; —C(NOH)NH₂; cyano; —P(O)R″R″; —OP(O)R″R″; halo; hydroxy; nitro; —NHSO₂(C₁-C₂)alkyl; —SO₃H; —SO₂(C₁-C₂)alkyl; —SO₂NR^{c}R^{d}; —SO₂NHC(O)(C₁-C₂)alkyl; and —B(OH)₂;

wherein in each occurrence:

R^{c}, R^{d} and R^{e} are independently selected from H and (C₁-C₂)alkyl;

R′ is independently selected from H, phenyl, (5-methyl-2-oxo-1,3-dioxol-4-yl)methyl:

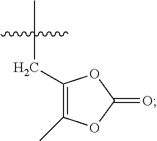 [SMILES: *Cc1oc(=O)oc1C]

and (C₁-C₄)alkyl, wherein said (C₁-C₄)alkyl is optionally substituted with 1-2 groups independently selected from phenyl, heteroaryl, —NR^{f}R^{f} wherein each R^{f} is independently selected from H and (C₁-C₄)alkyl, heterocycloalkyl optionally substituted with one oxo, —OC(O)O(C₁-C₄)alkyl, —OC(O)R^{g} wherein R^{g} is (C₁-C₄)alkyl or phenyl, and —C(O)NR^{h}R^{h} wherein R^{h} is independently selected from H and (C₁-C₄)alkyl;

R″ is independently selected from the group consisting of: —OH; (C₁-C₄)alkyl; (C₁—C₄)alkoxy; benzyloxy; phenoxy; (5-methyl-2-oxo-1,3-dioxol-4-yl)methoxy:

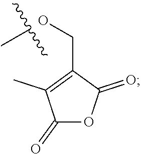

and —OCR^{a5}R^{b5}OC(O)R^{z}, wherein: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl;
provided that at least one of said 1-3 phenyl and heteroaryl substituents comprises at least 1 group selected from:

—CO₂R′, wherein R′ is phenyl, (5-methyl-2-oxo-1,3-dioxol-4-yl)methyl, or (C₁-C₄)alkyl, wherein said (C₁-C₄)alkyl is optionally substituted with 1-2 groups independently selected from phenyl, heteroaryl, NR^{f}R^{f} wherein each R^{f} is independently selected from H and (C₁-C₄)alkyl, heterocycloalkyl, —OC(O)O(C₁-C₄)alkyl, —OC(O)R^{g} wherein R^{g} is (C₁-C₄)alkyl or phenyl, and —C(O)NR^{h}R^{h} wherein R^{h} is independently selected from H and (C₁-C₄)alkyl;

—P(O)R″R″, wherein one R″ is selected from: (C₁-C₄)alkoxy; benzyloxy; phenoxy; (5-methyl-2-oxo-1,3-dioxol-4-yl)methoxy; and —OCR^{a5}R^{b5}OC(O)R^{z}, and the other R″ is selected from: OH; (C₁-C₄)alkyl; (C₁-C₄)alkoxy; benzyloxy; phenoxy; (5-methyl-2-oxo-1,3-dioxol-4-yl)methoxy; and —OCR^{a5}R^{b5}OC(O)R^{z} wherein in each occurrence: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl; and

—OP(O)R″R″, wherein one R″ is selected from: (C₁-C₄)alkoxy; benzyloxy; phenoxy; (5-methyl-2-oxo-1,3-dioxol-4-yl)methoxy; and —OCR^{a5}R^{b5}OC(O)R^{z}, and the other R″ is selected from: OH; (C₁-C₄)alkyl; (C₁-C₄)alkoxy; benzyloxy; phenoxy; (5-methyl-2-oxo-1,3-dioxol-4-yl)methoxy; and —OCR^{a5}R^{b5}OC(O)R^{z} wherein in each occurrence: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl).

In some embodiments of the compounds of formula (I):

R1 is selected from the group consisting of H, (C₁-C₄) straight chain alkyl, and (C₁-C₄) straight chain alkyl substituted with one hydroxy group;
R2 is selected from H, (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl, wherein heterocyclyl is a monocyclic ring having 5-6 ring atoms wherein 1-2 of the ring atoms are selected from nitrogen, oxygen and sulfur, and wherein said (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl are optionally substituted with 1-2 groups independently selected from (C₁-C₄)alkyl, (C₁-C₄)alkoxy, halo, and cyano; and
R3 is selected from phenyl and heteroaryl, wherein said phenyl and heteroaryl are substituted with 1-3 substituents independently selected from:
(C₁-C₆)alkyl, optionally substituted with 1-3 groups independently selected from: fluoro (e.g., —CF₃); —CO₂R′; —P(O)R″R″; NR^{a}R^{b} wherein R^{a} is selected from H and (C₁-C₄)alkyl and R^{b} is selected from (C₁-C₄)alkyl substituted with 1-3 groups independently selected from —CO₂R′ and —P(O)R″R″; and —C(O)NR^{a1}R^{b1} wherein R^{a1} and R^{b1} are independently selected from H and (C₁-C₄)alkyl, wherein the (C₁-C₄)alkyl is optionally substituted with 1-3 groups independently selected from hydroxy, —CO₂R′, —C(O)O(C₁-C₄)alkyl, and —P(O)R″R″;
cyclopropyl, optionally substituted with 1 —CO₂R′;
—C(O)NR^{a2}R^{b2} wherein R^{a2} and R^{b2} are independently selected from H and (C₁-C₄)alkyl, wherein the (C₁-C₄)alkyl is optionally substituted with 1-3 groups independently selected from hydroxy, —CO₂R′, —C(O)O(C₁-C₄)alkyl, —P(O)R″R″, NR^{c}R^{d} and N⁺R^{c}R^{d}R^{e};
(C₁-C₆)alkoxy, optionally substituted with 1-3 substituents independently selected from halo, hydroxy, —CO₂R′, (C₃-C₆)cycloalkyl, —C(O)NH₂ and pyrrolidinyl;
(C₃-C₆)cycloalkoxy, optionally substituted with 1-3 substituents independently selected from halo, hydroxy, and —CO₂R′;
—NR^{a3}R^{b3} wherein R^{a3} and R^{b3} are independently selected from H and (C₁-C₄)alkyl, wherein the (C₁-C₄)alkyl is optionally substituted with 1-3 groups independently selected from oxo and —CO₂R′;
—SR^{a4} wherein R^{a4} is selected from H and (C₁-C₄)alkyl;
—CO₂R′; —C(NOH)NH₂, cyano; —C(O)O(C₁-C₄)alkyl; —P(O)R″R″; —OP(O)R″R″; halo; hydroxy; nitro; —NHSO₂(C₁-C₂)alkyl; —SO₃H; —SO₂(C₁-C₂)alkyl; —SO₂NR^{c}R^{d}; —SO₂NHC(O)(C₁-C₂)alkyl; and —B(OH)₂;
wherein in each occurrence:
R^{c}, R^{d} and R^{e} are independently selected from H and (C₁-C₂)alkyl;
R′ is independently selected from H, (C₁-C₄)alkyl and benzyl;
R″ is independently selected from the group consisting of: OH; (C₁-C₄)alkyl; (C₁-C₄)alkoxy; benzyloxy; and —OCR^{a5}R^{b5}OC(O)R^{z}, wherein: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl;
provided that at least one of said 1-3 phenyl and heteroaryl substituents comprises at least 1 group selected from:
—CO₂R′, wherein R′ is (C₁-C₄)alkyl or benzyl;
—P(O)R″R″, wherein one R″ is selected from said (C₁-C₄)alkoxy; benzyloxy; and —OCR^{a5}R^{b5}OC(O)R^{z}, and the other R″ is selected from the group defined above for R″ (i.e., OH; (C₁-C₄)alkyl; (C₁-C₄)alkoxy; benzyloxy; and —OCR^{a5}R^{b5}OC(O)R^{z}, wherein: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl); and
—OP(O)R″R″, wherein one R″ is selected from said (C₁-C₄)alkoxy; benzyloxy; and —OCR^{a5}R^{b5}OC(O)R^{z}, and the other R″ is selected from the group defined above for R″ (i.e., OH; (C₁-C₄)alkyl; (C₁-C₄)alkoxy; benzyloxy; and —OCR^{a5}R^{b5}OC(O)R^{z}, wherein: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl).

In another aspect, the present invention relates to compounds of Formula (II):

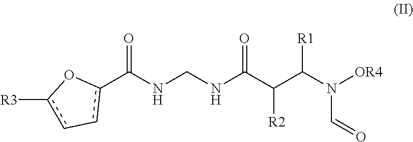

and salts thereof,
wherein:
R1 is selected from the group consisting of H, (C₁-C₄) straight chain alkyl, and (C₁-C₄) straight chain alkyl substituted with one hydroxy group;
R2 is selected from H, (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl, wherein heterocyclyl is a monocyclic ring having 5-6 ring atoms wherein 1-2 of the ring atoms are selected from nitrogen, oxygen and sulfur, and wherein said (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl are optionally substituted with 1-2 groups independently selected from (C₁-C₄)alkyl, (C₁-C₄)alkoxy, halo, and cyano; and
R3 is selected from: phenyl and heteroaryl, wherein said phenyl and heteroaryl are optionally substituted with 1-3 substituents independently selected from:

(C₁-C₆)alkyl, optionally substituted with 1-3 groups independently selected from: fluoro; —CO₂R′; —P(O)R″R″; —NR^{a}R^{b} wherein R^{a} is selected from H and (C₁-C₄)alkyl and R^{b} is selected from (C₁-C₄)alkyl substituted with 1-3 groups independently selected from —CO₂R′ and —P(O)R″R″; and —C(O)NR^{a1}R^{b1} wherein R^{a1} and R^{b1} are independently selected from H and (C₁-C₄)alkyl, wherein the (C₁-C₄)alkyl is optionally substituted with 1-3 groups independently selected from hydroxy, —CO₂R′, and —P(O)R″R″;

cyclopropyl, optionally substituted with one —CO₂R′;

—C(O)NR^{a2}R^{b2} wherein R^{a2} and R^{b2} are independently selected from H and (C₁-C₄)alkyl, wherein the (C₁-C₄)alkyl is optionally substituted with 1-3 groups independently selected from hydroxy, —CO₂R′, —P(O)R″R″, —NR^{c}R^{d} and —N⁺R^{c}R^{d}R^{e};

(C₁-C₆)alkoxy, optionally substituted with 1-3 substituents independently selected from halo, hydroxy, —CO₂R′, (C₃-C₆)cycloalkyl, —C(O)NH₂ and pyrrolidinyl;

(C₃-C₆)cycloalkoxy, optionally substituted with 1-3 substituents independently selected from halo, hydroxy, and —CO₂R′;

—NR^{a3}R^{b3} wherein R^{a3} and R^{b3} are independently selected from H and (C₁-C₄)alkyl, wherein the (C₁-C₄)alkyl is optionally substituted with 1-3 groups independently selected from oxo and —CO₂R′;

—SR^{a4} wherein R^{a4} is selected from H and (C₁-C₄)alkyl;

—CO₂R′; —C(NOH)NH₂; cyano; —P(O)R″R″; —OP(O)R″R″; halo; hydroxy; nitro; —NHSO₂(C₁-C₂)alkyl; —SO₃H; —SO₂(C₁-C₂)alkyl; —SO₂NR^{c}R^{d}; —SO₂NHC(O)(C₁-C₂)alkyl; and —B(OH)₂;

wherein in each occurrence:

R^{c}, R^{d} and R^{e} are independently selected from H and (C₁-C₂)alkyl;

R′ is independently selected from H, phenyl, (5-methyl-2-oxo-1,3-dioxol-4-yl)methyl, and (C₁-C₄)alkyl, wherein said (C₁-C₄)alkyl is optionally substituted with 1-2 groups independently selected from phenyl, heteroaryl, —NR^{f}R^{f} wherein each R^{f} is independently selected from H and (C₁-C₄)alkyl, heterocycloalkyl optionally substituted with one oxo, —OC(O)O(C₁-C₄)alkyl, —OC(O)R^{g} wherein R^{g} is (C₁-C₄)alkyl or phenyl, and —C(O)NR^{h}R^{h} wherein R^{h} is independently selected from H and (C₁-C₄)alkyl;

R″ is independently selected from the group consisting of —OH; (C₁-C₄)alkyl; (C₁-C₄)alkoxy; benzyloxy; phenoxy; (5-methyl-2-oxo-1,3-dioxol-4-yl)methoxy; and —OCR^{a5}R^{b5}OC(O)R^{z} wherein R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl; and

R4 is selected from the groups A-G defined below:

A. —C(O)R^{x} where R^{x} is selected from:

a. —NRⁱRⁱ where one Rⁱ is H or (C₁-C₄)alkyl, and the other is a —C₂H₄NH-tripeptide group; or where each Rⁱ is independently selected from H, phenyl, (C₁-C₄)alkyl optionally substituted with 1-2 hydroxy or phenyl groups, —C(O)(C₁-C₄)alkyl, —C(O)(C₁-C₈)alkoxy, and phenyl;

b. phenyl, optionally substituted with 1-2 groups independently selected from:

hydroxy;

halo;

(C₁-C₄)alkyl optionally substituted with one group NR^{v}R^{v} where each R^{v} is independently selected from H and (C₁-C₄)alkyl;

—CO₂H;

(C₁-C₄)alkoxy;

—PO₃H₂;

—OPO₃H₂;

—OC(O)(C₁-C₄)alkyl;

—NRⁱRⁱ where each Rⁱ is independently selected from H and (C₁-C₄)alkyl wherein the (C₁-C₄)alkyl is optionally substituted with 1-4 groups independently selected from —NHMe and —NMe₂;

—CF₃;

—OCF₃;

—NO₂;

—B(OH)₂;

—OC(O)NR^{k}R^{k} wherein each R^{k} is independently selected from H and (C₁-C₄)alkyl;

—(CH₂)₀₋₁ heterocycloalkyl containing 1-2 ring nitrogens and/or 1-2 ring oxygens, optionally substituted with one oxo group;

heteroaryl optionally substituted with a C-linked pyranose group:

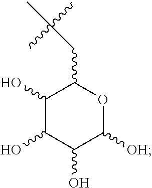 [SMILES: *CC1OC(O)C(O)C(O)C1O]

—O(C₁-C₄)alkyl substituted with one group selected from —NR^{l}R^{l} wherein each R^{l} is independently selected from H and (C₁-C₄)alkyl, heterocycloalkyl, heteroaryl wherein heteroaryl is optionally substituted with one C-linked pyranose group:

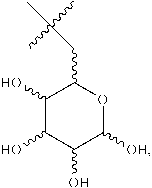 [SMILES: *CC1OC(O)C(O)C(O)C1O]

and —O(C₁-C₄)alkyl substituted with one —OH group;

—NR^{m}SO₂R^{m} wherein each R^{m} is independently selected from H and (C₁-C₄)alkyl;

—OCH₂OPO₃H₂;

an O-linked pyranose group selected from

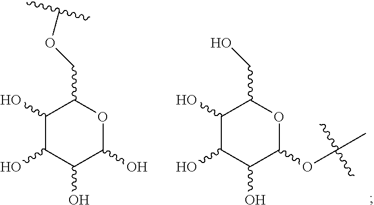 [SMILES: *OCC1OC(O)C(O)C(O)C1O ; *OC1OC(CO)C(O)C(O)C1O ; ";"]

a group

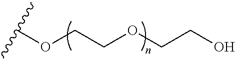

where n=an integer of from 1-20;

a group

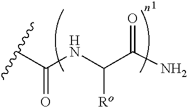

where n¹=an integer of from 1-5 and R^{o} is an amino acid side chain; and

a group

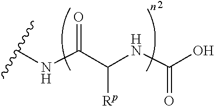

where n²=an integer of from 1-5 and R^{p} is an amino acid side chain;

c. bicyclic heteroaryl containing a phenyl ring moiety fused to a heterocycloalkyl ring moiety, wherein the heterocycloalkyl ring has 1-4 ring atoms selected from nitrogen and oxygen and is optionally substituted with 1-2 (C₁-C₄)alkyl groups;

d. a group:

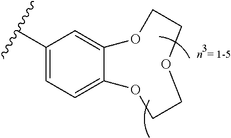

where n³ is an integer of from 1-5;

e. —NR^{q}C₂H₄OC(O)R^{r} where R^{q} is H or (C₁-C₄)alkyl and R^{r} is selected from: (C₁-C₅)alkyl; phenyl optionally substituted with 1-2 groups independently selected from hydroxy, halo, (C₁-C₄)alkyl, —CO₂H, (C₁-C₄)alkoxy, —PO₃H₂, and —OC(O)Me; pyridyl optionally substituted with 1-2 groups independently selected from hydroxy, halogen, (C₁-C₄)alkyl, —CO₂H, (C₁-C₄)alkoxy, —PO₃H₂, and —OC(O)Me; and (C₁-C₄) alkoxy;

f. (C₁-C₁₀)alkyl, optionally substituted with 1-2 phenyl groups;

g. (C₁-C₄)alkoxy;

h. monocyclic heteroaryl comprising 5 or 6 ring atoms comprising 1-3 heteroatoms selected from N and O, optionally substituted with 1-2 groups independently selected from hydroxy, halogen, (C₁-C₄)alkyl, —CO₂H, (C₁-C₄)alkoxy, —PO₃H₂, and —OC(O)Me;

i. naphthyl;

j. (C₁-C₄)alkyl optionally substituted with one —NR^{s}R^{s} where R^{s} is independently selected from: H, Me, and phenoxy optionally substituted with 1-2 groups independently selected from halogen, (C₁-C₄)alkyl, —CO₂H, (C₁-C₄)alkoxy, —PO₃H₂, and —OC(O)Me;

k. piperidinyl;

l. pyrrolidinyl; and

m. (C₃-C₆)cycloalkyl optionally substituted with one (C₁-C₄)alkyl group;

B. —CR^{a6}R^{b6}OR^{y} wherein each of R^{a6} and R^{b6} is independently selected from H, (C₁-C₄)alkyl, phenyl, and (C₃-C₆)cycloalkyl, or R^{a6} and R^{b6} together with the carbon to which they are attached form a (C₃-C₆)cycloalkyl, and R^{y} is selected from:
a. —COR^{z1} where R^{z1} is selected from:
   —NR^{t}R^{t} where one R^{t} is H or (C₁-C₄)alkyl, and the other is a —C₂H₄NH-tripeptide group;
   phenyl optionally substituted with 1-2 groups independently selected from hydroxy, halo, (C₁-C₄)alkyl, —CO₂H, (C₁-C₄)alkoxy, —PO₃H₂, and —OC(O)Me;
   benzyl;
   (C₁-C₅)alkyl;
   (C₁-C₄)alkoxy;
   (C₁-C₄)alkyl optionally substituted with one —NR^{u}R^{u} where R^{u} is independently selected from H, Me, and phenoxy optionally substituted with 1-2 groups independently selected from halogen, (C₁-C₄)alkyl, —CO₂H, (C₁-C₄)alkoxy, —PO₃H₂, and —OC(O)Me;
   pyridyl optionally substituted with 1-2 groups independently selected from hydroxy, halogen, (C₁-C₄)alkyl, —CO₂H, (C₁-C₄)alkoxy, —PO₃H₂, and —OC(O)Me;
   pyrrolidinyl; and

### —CH═CHCO₂H;

b. —P(O)R^{z2}R^{z2} where each R^{z2} is independently selected from:

### H;

### —OH;

(C₁-C₅)alkyl;
(C₁-C₅)alkoxy;
phenoxy optionally substituted with 1-2 groups independently selected from halo, —OMe, and —OEt;
benzyloxy;
—NR^{a7}R^{b7} wherein R^{a7} is selected from H and (C₁-C₄)alkyl and R^{b7} is selected from (C₁—C₄)alkyl substituted with 1-3 groups independently selected from: —CO₂R′^{A}, where R′^{A} is independently selected from H, (C₁-C₄)alkyl and benzyl; phenyl; —OH; —SH; —SMe; and phenyl substituted with one —OH, —NH₂, —C(O)NH₂, imidazol-4-yl, or indol-3-yl;
—NR^{a8}R^{b8} wherein R^{a8} and R^{b8} together with the nitrogen to which they are attached form a 4 to 6 membered heterocycloalkyl, optionally substituted with one —CO₂R′^{B} group, where R′^{B} is selected from H, (C₁-C₄)alkyl and benzyl;
—OCR^{a9}R^{b9}OC(O)R^{p1} wherein R^{a9} and R^{b9} are independently selected from H, (C₁-C₄)alkyl, phenyl, (C₁-C₄)alkoxy, and (C₃-C₆)cycloalkyl, or R^{a9} and R^{b9} together with the carbon to which they are attached form a (C₃-C₆)cycloalkyl; and R1 is selected from (C₁-C₅)alkyl, (C₁-C₅)alkoxy, and phenyl;
c. a group which is:

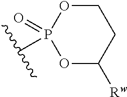

wherein R^{w} is selected from: phenyl optionally substituted with one halo, and pyridyl; and
d. a group selected from:

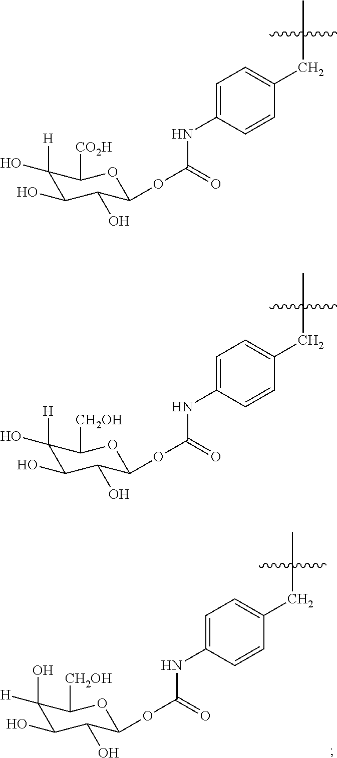 [SMILES: *Cc1ccc(NC(=O)OC2OC(C(=O)O)C(O)C(O)C2O)cc1 ; *Cc1ccc(NC(=O)OC2OC(CO)C(O)C(O)C2O)cc1 ; *Cc1ccc(NC(=O)OC2OC(CO)C(O)C(O)C2O)cc1 ; ";"]

and

C. (5-methyl-2-oxo-1,3-dioxol-4-yl)methyl:

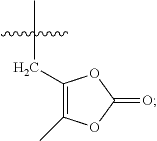 [SMILES: *Cc1oc(=O)oc1C]

D. —P(O)R^{z3}R^{z3} where each R^{z3} is independently selected from:

### H;

### —OH;

(C₁-C₅)alkyl;
(C₁-C₅)alkoxy;
phenoxy optionally substituted with 1-2 groups independently selected from halo, —OMe, and —OEt;
benzyloxy;
—NR^{a10}R^{b10} wherein R^{a10} is selected from H and (C₁-C₄)alkyl and R^{b10} is selected from (C₁-C₄)alkyl substituted with 1-3 groups independently selected from: —CO₂R′^{C}, where R′^{C} is independently selected from H, (C₁-C₄)alkyl and benzyl; phenyl; —OH; —SH; —SMe; and phenyl substituted with one group selected from —OH, —NH₂, —C(O)NH₂, imidazol-4-yl, and indol-3-yl;
—NR^{a11}R^{b11} wherein R^{a11} and R^{b11} together with the nitrogen to which they are attached form a 4 to 6 membered heterocycloalkyl optionally substituted with one —CO₂R′^{D} group, where R′^{D} is selected from H, (C₁-C₄)alkyl and benzyl; and
—OCR^{a12}R^{b12}OC(O)R^{p2} wherein R^{a12} and R^{b12} are independently selected from H, (C₁-C₄)alkyl, phenyl, (C₁-C₄)alkoxy, and (C₃-C₆)cycloalkyl, or R^{a12} and R^{b12} together with the carbon to which they are attached form a (C₃-C₆)cycloalkyl; and R^{p2} is selected from (C₁-C₅)alkyl, (C₁-C₅)alkoxy, and phenyl;

E. a group which is:

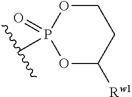

wherein R^{w1} is selected from: phenyl, phenyl substituted with one halo, and pyridyl;

F. a group selected from:

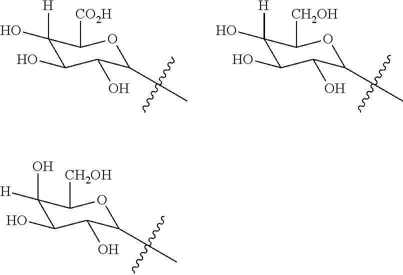 [SMILES: *C1OC(C(=O)O)C(O)C(O)C1O ; *C1OC(CO)C(O)C(O)C1O ; *C1OC(CO)C(O)C(O)C1O]

and

G. a group selected from:

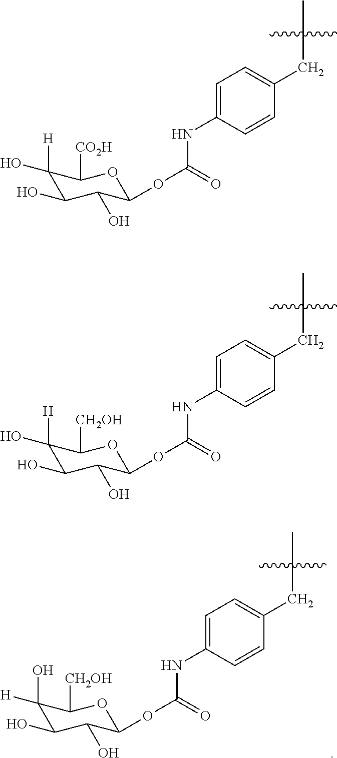 [SMILES: *Cc1ccc(NC(=O)OC2OC(C(=O)O)C(O)C(O)C2O)cc1 ; *Cc1ccc(NC(=O)OC2OC(CO)C(O)C(O)C2O)cc1 ; *Cc1ccc(NC(=O)OC2OC(CO)C(O)C(O)C2O)cc1 ; "."]

In some embodiments of the compounds of formula (II):

R1 is selected from the group consisting of H, (C₁-C₄) straight chain alkyl, and (C₁-C₄) straight chain alkyl substituted with one hydroxy group;
R2 is selected from H, (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl, wherein heterocyclyl is a monocyclic ring having 5-6 ring atoms wherein 1-2 of the ring atoms are selected from nitrogen, oxygen and sulfur, and wherein said (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl are optionally substituted with 1-2 groups independently selected from (C₁-C₄)alkyl, (C₁-C₄)alkoxy, halo, and cyano; and
R3 is selected from: phenyl and heteroaryl, wherein said phenyl and heteroaryl are optionally substituted with 1-3 substituents independently selected from:
(C₁-C₆)alkyl, optionally substituted with 1-3 groups independently selected from: fluoro (e.g., —CF₃); —CO₂R′; —P(O)R″R″; NR^{a}R^{b} wherein R^{a} is selected from H and (C₁-C₄)alkyl and R^{b} is selected from (C₁-C₄)alkyl substituted with 1-3 groups independently selected from] —CO₂R′ and —P(O)R″R″; and —C(O)NR^{a1}R^{b1} wherein R^{a1} and R^{b1} are independently selected from H and (C₁-C₄)alkyl, wherein the (C₁-C₄)alkyl is optionally substituted with 1-3 groups independently selected from hydroxy, —CO₂R′, —C(O)O(C₁-C₄)alkyl, and —P(O)R″R″;
cyclopropyl, optionally substituted with 1 —CO₂R′;
—C(O)NR^{a2}R^{b2} wherein R^{a2} and R^{b2} are independently selected from H and (C₁-C₄)alkyl, wherein the (C₁-C₄)alkyl is optionally substituted with 1-3 groups independently selected from hydroxy, —CO₂R′, —C(O)O(C₁-C₄)alkyl, —P(O)R″R″, NR^{c}R^{d} and N⁺R^{c}R^{d}R^{e};
(C₁-C₆)alkoxy, optionally substituted with 1-3 substituents independently selected from halo, hydroxy, —CO₂R′, (C₃-C₆)cycloalkyl, —C(O)NH₂ and pyrrolidinyl;
(C₃-C₆)cycloalkoxy, optionally substituted with 1-3 substituents independently selected from halo, hydroxy, and —CO₂R′;
—NR^{a3}R^{b3} wherein R^{a3} and R^{b3} are independently selected from H and (C₁-C₄)alkyl, wherein the (C₁-C₄)alkyl is optionally substituted with 1-3 groups independently selected from oxo and —CO₂R′;
—SR^{a4} wherein R^{a4} is selected from H and (C₁-C₄)alkyl;
CO₂R′; —C(NOH)NH₂, cyano; —C(O)O(C₁-C₄)alkyl; —P(O)R″R″; —OP(O)R″R″; halo; hydroxy; nitro; —NHSO₂(C₁-C₂)alkyl; —SO₃H; —SO₂(C₁-C₂)alkyl; —SO₂NR^{c}R^{d}; —SO₂NHC(O)(C₁-C₂)alkyl; and —B(OH)₂;
wherein in each occurrence:
R^{c}, R^{d} and R^{e} are independently selected from H and (C₁-C₂)alkyl;
R′ is independently selected from H, (C₁-C₄)alkyl and benzyl;
R″ is independently selected from the group consisting of —OH; (C₁-C₄)alkyl; (C₁-C₄)alkoxy; benzyloxy; and —OCR^{a5}R^{b5}OC(O)R^{z} wherein R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached, form a (C₃-C₆)cycloalkyl; and R^{z} is selected from (C₁-C₅)alkyl, (C₁-C₅)alkoxy, and phenyl;
R4 is selected from the groups A-G defined below:

A. —C(O)R^{x} where R^{x} is selected from:

—NRⁱRⁱ where one Rⁱ is H or (C₁-C₄)alkyl, and the other is a —C₂H₄NH-tripeptide group [e.g., —C₂H₄NH(Lys-Leu-DVal) or —C₂H₄NH(Lys-Phe-DAla)];
phenyl, optionally substituted with 1-2 groups independently selected from hydroxy, halogen, (C₁-C₄)alkyl, —CO₂H, (C₁-C₄)alkoxy, —PO₃H₂, and —OC(O)Me;
—NR^{q}C₂H₄OC(O)R^{r} where R^{q} is H or (C₁-C₄)alkyl and R^{r} is selected from: (C₁-C₅)alkyl; phenyl optionally substituted with 1-2 groups independently selected from hydroxy, halogen, (C₁-C₄)alkyl, —CO₂H, (C₁-C₄)alkoxy, —PO₃H₂, and —OC(O)Me; pyridyl optionally substituted with 1-2 groups independently selected from hydroxy, halogen, (C₁-C₄)alkyl, —CO₂H, (C₁-C₄)alkoxy, —PO₃H₂, and —OC(O)Me; and (C₁-C₄) alkoxy;
benzyl;
(C₁-C₅)alkyl;
(C₁-C₄)alkoxy;
(C₁-C₄)alkyl optionally substituted with one —NR^{s}R^{s} where R^{s} is independently selected from H, Me, and phenoxy optionally substituted with 1-2 groups independently selected from halogen, (C₁-C₄)alkyl, —CO₂H, (C₁-C₄)alkoxy, —PO₃H₂, and —OC(O)Me;
pyrrolidinyl;
pyridyl optionally substituted with 1-2 groups independently selected from hydroxy, halogen, (C₁-C₄)alkyl, —CO₂H, (C₁-C₄)alkoxy, —PO₃H₂, and —OC(O)Me; and
—NRⁱRⁱ where each Rⁱ is independently selected from H, Me, CH₂OH, and C(O)Me;

B. —CR^{a6}R^{b6}OR^{y} wherein each of R^{a6} and R^{b6} is independently selected from H, (C₁-C₄)alkyl, phenyl, and (C₃-C₆)cycloalkyl, or R^{a6} and R^{b6} together with the carbon to which they are attached, form a (C₃-C₆)cycloalkyl, and R^{y} is selected from:
a. —COR^{z1} where R^{z1} is selected from:
   —NR^{t}R^{t} where one R^{t} is H or (C₁-C₄)alkyl, and the other is a —C₂H₄NH-tripeptide group [e.g., —C₂H₄NH(Lys-Leu-DVal) or —C₂H₄NH(Lys-Phe-DAla)];
   phenyl optionally substituted with 1-2 groups independently selected from hydroxy, halogen, (C₁-C₄)alkyl, —CO₂H, (C₁-C₄)alkoxy, —PO₃H₂, and —OC(O)Me;
   benzyl;
   (C₁-C₅)alkyl;
   (C₁-C₄)alkoxy;
   (C₁-C₄)alkyl optionally substituted with one —NR^{u}R^{u} where R^{u} is independently selected from H, Me, and phenoxy optionally substituted with 1-2 groups independently selected from halogen, (C₁-C₄)alkyl, —CO₂H, (C₁-C₄)alkoxy, —PO₃H₂, and —OC(O)Me;
   pyridyl optionally substituted with 1-2 groups independently selected from hydroxy, halogen, (C₁-C₄)alkyl, —CO₂H, (C₁-C₄)alkoxy, —PO₃H₂, and —OC(O)Me;
   pyrrolidinyl; and

### —CHCHCO₂H;

b. —P(O)R^{z2}R^{z2} where each R^{z2} is independently selected from:

### H;

### OH;

(C₁-C₅)alkyl;
(C₁-C₅)alkoxy;
phenoxy;
phenoxy substituted with 1-2 groups independently selected from halo, OMe, and OEt;
benzyloxy;
NR^{a7}R^{b7} wherein R^{a7} is selected from H and (C₁-C₄)alkyl and R^{b7} is selected from (C₁-C₄)alkyl substituted with 1-3 groups independently selected from: —CO₂R′^{A}, where R′^{A} is independently selected from H, (C₁-C₄)alkyl and benzyl; (C₁-C₄)alkyl; phenyl; OH; SH; SMe; and phenyl substituted with one OH, NH₂, C(O)NH₂, 4-imidazole, or 3-indole group;
NR^{a8}R^{b8} wherein R^{a8} and R^{b8} together with the nitrogen to which they are attached form a 4-6 membered heterocycloalkyl optionally substituted with one —CO₂R′^{B} group, where R′^{B} is selected from H, (C₁-C₄)alkyl and benzyl;
—OCR^{a9}R^{b9}OC(O)R^{p1} wherein R^{a9} and R^{b9} are independently selected from H, (C₁-C₄)alkyl, phenyl, (C₁-C₄)alkoxy, and (C₃-C₆)cycloalkyl, or R^{a9} and R^{b9} together with the carbon to which they are attached, form a (C₃-C₆)cycloalkyl; and R^{p1} is selected from (C₁-C₅)alkyl, (C₁—C₅)alkoxy, and phenyl;
c. a group:

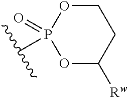

wherein R^{w} is selected from: phenyl; phenyl substituted with one halo; and pyridyl; and
d. a group selected from:

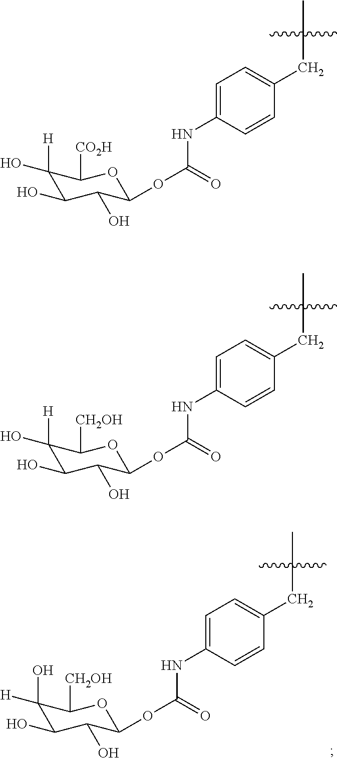 [SMILES: *Cc1ccc(NC(=O)OC2OC(C(=O)O)C(O)C(O)C2O)cc1 ; *Cc1ccc(NC(=O)OC2OC(CO)C(O)C(O)C2O)cc1 ; *Cc1ccc(NC(=O)OC2OC(CO)C(O)C(O)C2O)cc1 ; ";"]

C. (5-methyl-2-oxo-1,3-dioxol-4-yl)methyl:

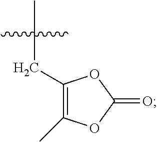 [SMILES: *Cc1oc(=O)oc1C]

D. —P(O)R^{z3}R^{z3} where each R^{z3} is independently selected from:

### H;

### OH;

(C₁-C₅)alkyl;
(C₁-C₅)alkoxy;
phenoxy;
phenoxy substituted with 1-2 groups independently selected from halo, OMe, and OEt;
benzyloxy;
NR^{a10}R^{b10} wherein R^{a10} is selected from H and (C₁-C₄)alkyl and R^{b10} is selected from (C₁-C₄)alkyl substituted with 1-3 groups independently selected from: —CO₂R′^{C}, where R′^{C} is independently selected from H, (C₁-C₄)alkyl and benzyl; (C₁-C₄)alkyl; phenyl; OH; SH; SMe; and phenyl substituted with one group selected from OH, NH₂, C(O)NH₂, 4-imidazole, and 3-indole;
NR^{a11}R^{b11} wherein R^{a11} and R^{b11} together with the nitrogen to which they are attached form a 4-6 membered heterocycloalkyl optionally substituted with one —CO₂R′^{D} group, where R′^{D} is selected from H, (C₁-C₄)alkyl and benzyl;
—OCR^{a12}R^{b12}OC(O)R^{p2} wherein R^{a12} and R^{b12} are independently selected from H, (C₁-C₄)alkyl, phenyl, (C₁-C₄)alkoxy, and (C₃-C₆)cycloalkyl, or R^{a12} and R^{b12} together with the carbon to which they are attached, form a (C₃-C₆)cycloalkyl; and R^{p2} is selected from (C₁-C₅)alkyl, (C₁-C₅)alkoxy, and phenyl;

E. a group:

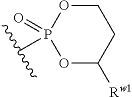

wherein R^{w1} is selected from: phenyl; phenyl substituted with one halo; and pyridyl;

F. a group selected from:

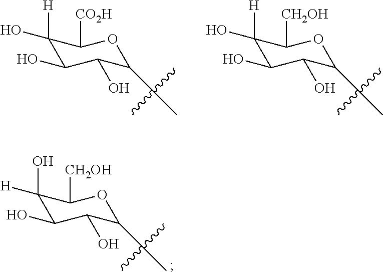 [SMILES: *C1OC(C(=O)O)C(O)C(O)C1O ; *C1OC(CO)C(O)C(O)C1O ; *C1OC(CO)C(O)C(O)C1O ; ";"]

and

G. a group selected from:

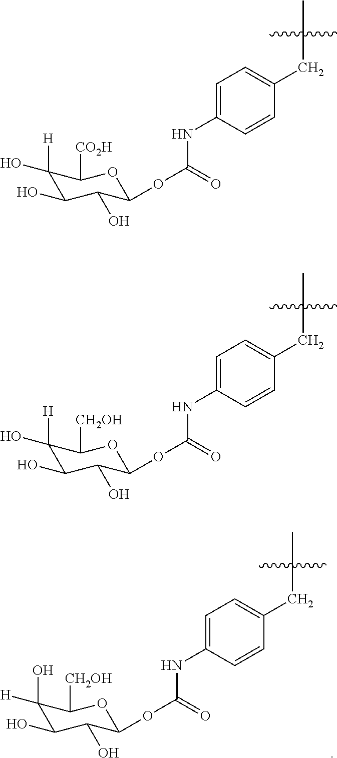 [SMILES: *Cc1ccc(NC(=O)OC2OC(C(=O)O)C(O)C(O)C2O)cc1 ; *Cc1ccc(NC(=O)OC2OC(CO)C(O)C(O)C2O)cc1 ; *Cc1ccc(NC(=O)OC2OC(CO)C(O)C(O)C2O)cc1 ; "."]

The compounds according to Formula (I) and (II), or salts, particularly pharmaceutically acceptable salts, thereof, are BMP1, TLL1 and/or TLL2 inhibitors and/or are capable of generating a BMP1, TLL1 and/or TLL2 inhibitor in vivo, ex vivo or in vitro, e.g. upon administration to a subject, and/or upon combination with one or more suitable excipients before administration to a subject. In some embodiments, the compound generates a BMP1, TLL1 and/or TLL2 inhibitor disclosed in PCT application no. PCT/IB2015/050179 or PCT publication no. WO2015/104684.

Accordingly, the present invention is also directed to a method of inhibiting BMP1, TLL1 and/or TLL2 which method comprises contacting a biological material comprising the protein(s) with a compound according to Formula (I) or (II), or a salt, particularly a pharmaceutically acceptable salt, thereof.

The invention is further directed to a method of treating a disease associated with BMP1, TLL1 and/or TLL2 activity in a subject (e.g., a human or other mammal, particularly a human) in need thereof, including for example treatment of a disease where inhibition of BMP1, TLL1 and/or TLL2 is of therapeutic benefit, which comprises administering to the subject a therapeutically effective amount of a compound according to Formula (I) or (II), or a salt thereof, particularly a pharmaceutically acceptable salt thereof. This invention also provides a compound of Formula (I) or (II), or a salt thereof, particularly a pharmaceutically acceptable salt thereof, for use in therapy, e.g. as an active therapeutic substance in the treatment of a disease associated with BMP1, TLL1 and/or TLL2 activity. The invention also provides for the use of a compound of Formula (I) or (II), or a salt thereof, particularly a pharmaceutically acceptable salt thereof, in the manufacture of a medicament for use in the treatment of a disease associated with BMP1, TLL1 and/or TLL2 activity. The present invention is further directed to a pharmaceutical composition comprising a compound according to Formula (I) or (II), or a salt thereof, particularly a pharmaceutically acceptable salt thereof, and one or more pharmaceutically acceptable excipients. Particularly, this invention is directed to a pharmaceutical composition for the treatment of a disease associated with BMP1, TLL1 and/or TLL2 activity, where the composition comprises a compound according to Formula (I) or (II), or a salt thereof, particularly a pharmaceutically acceptable salt thereof, and one or more pharmaceutically acceptable excipients.

The invention is also directed to a composition, e.g., a pharmaceutical composition, formed by combining a compound of the invention with at least one pharmaceutically acceptable excipient, as well as methods and uses analogous to the above, comprising administration or use of the composition.

In some embodiments, the disease associated with BMP1, TLL1 and/or TLL2 activity is selected from those associated with pathological fibrotic conditions in body organs or tissues, e.g., such conditions of the: heart (e.g., myocardial infarction (“MI”), heart failure (e.g., heart failure with reduced ejection fraction, heart failure with preserved ejection fraction), cardiac arrhythmias (e.g., atrial fibrillation), hypertrophic cardiomyopathy), lung (e.g. chronic obstructive pulmonary disease (“COPD”), idiopathic pulmonary fibrosis (“IPF”)), kidney (e.g. diabetic nephropathy, post-acute kidney injury, chronic kidney disease (“CKD”), delayed graft function post-transplantation), liver (e.g. liver cirrhosis, non-alcoholic steatohepatitis (“NASH”)), eye (e.g. glaucoma, corneal scarring), skeletal muscle (e.g. muscular dystrophies, including Duchenne, Becker, limb-girdle, congenital, facioscapulohumeral, myotonic, oculopharyngeal, distal, and Emery-Dreifuss), skin (e.g. keloids, wound healing, adhesions, hypertrophic scarring and other scarring, e.g., associated with burns, surgery or other trauma), the vasculature (e.g. stroke, and collagen vascular diseases such as systemic lupus erythematosus, rheumatoid arthritis and scleroderma), and the nervous system (e.g. spinal cord injury, multiple sclerosis). In some embodiments, the disease associated with BMP1, TLL1 and/or TLL2 activity is selected from muscular diseases characterized by reduced muscle function and/or mass, e.g., muscular dystrophy (e.g., Duchenne, Becker, limb-girdle, congenital, facioscapulohumeral, myotonic, oculopharyngeal, distal, and Emery-Dreifuss), sarcopenia, and cachexia associated with, e.g., heart failure, CKD, COPD, cancer, or old age.

Other aspects of the present invention will be understood in light of this disclosure.

### DETAILED DESCRIPTION OF THE INVENTION

The alternative definitions for the various groups and substituent groups of Formula (I) and (II) provided throughout the specification are intended to particularly describe each compound species disclosed herein, individually, as well as groups of one or more compound species. The scope of this invention includes any combination of these group and substituent group definitions. The compounds of the invention are only those which are contemplated to be “chemically stable” as will be appreciated by those skilled in the art.

As used herein, the term “alkyl” represents a saturated hydrocarbon moiety which, unless otherwise stated, may be straight or branched. The terms “C₁-C₂ alkyl”, “C₁-C₃ alkyl”, “C₁-C₄ alkyl”, “C₁-C₅ alkyl”, “C₁-C₆ alkyl”, “C₁-C₁₀ alkyl”, and “C₁-C₁₁ alkyl” refer to an alkyl group or moiety containing 1-2, 1-3, 1-4, 1-5, 1-6, 1-10 or 1-11 carbon atoms respectively. Exemplary alkyls include, but are not limited to methyl (Me), ethyl (Et), n-propyl (Pr), isopropyl (iPr), n-butyl, s-butyl, isobutyl, t-butyl (butyl may be abbreviated as “but” or “But”), pentyl (also known as n-pentyl), and 2-ethylbutyl, as well as hexyl, heptyl, octyl, nonyl, decyl and undecyl, including the branched isomers of these groups.

As used herein, the term “cycloalkyl” refers to a non-aromatic, saturated, cyclic hydrocarbon ring moiety. The term “(C₃-C₆)cycloalkyl” refers to a non-aromatic cyclic hydrocarbon ring moiety having three to six ring carbon atoms. Exemplary “(C₃-C₆)cycloalkyl” groups include cyclopropyl, cyclobutyl, cyclopentyl, and cyclohexyl.

“Alkoxy” refers to an alkyl radical attached through an oxygen linking atom. The terms “(C₁-C₄)alkoxy”, “(C₁-C₅)alkoxy”, “(C₁-C₆)alkoxy” and “(C₁-C₈)alkoxy” refer to a straight- or branched-chain hydrocarbon radical containing 1-4, 1-5, 1-6 or 1-8 carbon atoms respectively, attached through an oxygen linking atom. “(C₁-C₄)alkoxy”, “(C₁-C₅)alkoxy”, “(C₁-C₆)alkoxy” and “(C₁-C₈)alkoxy” may be alternatively designated as —O(C₁-C₄ alkyl), —O(C₁-C₅ alkyl), —O(C₁-C₆ alkyl), and —O(C₁-C₈ alkyl) respectively. Exemplary alkoxy groups include, but are not limited to, methoxy (alternatively, “OMe”), ethoxy (alternatively, “OEt”), n-propoxy, isopropoxy, n-butoxy, s-butoxy, isobutoxy, t-butoxy, pentoxy, hexoxy, and octyloxy, including the branched isomers of these groups.

“Cycloalkoxy” refers to a cycloalkyl radical attached through an oxygen linking atom.

The term “(C₃-C₆)cycloalkoxy” refers to a cycloalkyl radical having 3 to 6 ring carbon atoms, attached through an oxygen linking atom. “(C₃-C₆)cycloalkoxy” may be alternatively designated as —O(C₃-C₆)cycloalkyl. Exemplary cycloalkoxy groups include cyclopropyloxy, cyclobutyloxy, cyclpentyloxy, and cyclohexyloxy.

A heterocyclic (alternatively referred to as heterocyclyl) group or moiety is a mono- or bi-cyclic group or moiety having as ring members atoms of at least two different elements (carbon and one or more of nitrogen, oxygen and/or sulfur). The ring(s) may be saturated or partially unsaturated (non-aromatic) or fully unsaturated (aromatic). Heterocyclic encompasses heterocycloalkyl and heteroaryl. For example, heterocyclyl may be a cyclic group or moiety having 5-10 ring atoms (i.e. “5-10 membered”) wherein 1-4 of the ring atoms are heteroatoms selected from nitrogen, oxygen and sulfur, e.g., a monocyclic ring having 5-6 ring atoms wherein 1-2 of the ring atoms are heteroatoms selected from nitrogen, oxygen and sulfur, or a bicyclic ring having 9-10 ring atoms wherein 1-4 of the ring atoms are heteroatoms selected from nitrogen, oxygen and sulfur.

“Heterocycloalkyl” represents a group or moiety which is a non-aromatic, monocyclic radical, which is saturated or partially unsaturated, having 5-6 ring atoms wherein 1-2 of the ring atoms are heteroatoms selected from nitrogen, oxygen and sulfur. Illustrative examples of heterocycloalkyl groups include, but are not limited to, piperidyl (or piperidinyl), piperazinyl, pyrrolidinyl, morpholinyl, tetrahydrofuryl (or tetrahydrofuranyl), tetrahydropyranyl, tetrahydrothienyl, and thiomorpholinyl, including the various position isomers of the foregoing moieties.

“Heteroaryl” refers to a mono- or bi-cyclic group or moiety wherein at least one ring is aromatic, having 5-10 ring atoms wherein 1-4 of the ring atoms are heteroatoms selected from nitrogen, oxygen and sulfur. In bicyclic heteroaryl, at least one ring is aromatic and the other ring may be aromatic, or saturated or unsaturated non-aromatic, and at least one ring is heterocyclic and the other ring may be heterocyclic or carbocyclic. Thus, this term encompasses but is not limited to bicyclic heterocyclic compounds containing at least one aromatic carbocyclic or heterocylic ring moiety, e.g., a phenyl ring moiety fused to a heterocycloalkyl ring moiety. Illustrative examples of heteroaryls include, but are not limited to, thienyl, pyrrolyl, imidazolyl, pyrazolyl, furyl (or furanyl), isothiazolyl, isoxazolyl, oxazolyl, oxadiazolyl, thiazolyl, pyridyl (or pyridinyl), pyrazinyl, pyrimidinyl, pyridazinyl, triazinyl, triazolyl, tetrazolyl, indazolyl, benzothienyl, benzofuranyl, benzothiazolyl, benzimidazolyl, benzoxazolyl, benzooxazinyl, benzoxadiazolyl, benzothiadiazolyl, benzotriazolyl, 2,3-dihydrobenzoisothiazolyl, and 1,1-dioxido-2,3-dihydrobenzoisothiazolyl (e.g., 1,1-dioxido-2,3-dihydrobenzo[d]isothiazolyl), dioxolyl, 2,3-dihydrobenzo[b][1,4]dioxinyl, including the various position isomers of the foregoing moieties.

In some embodiments, compounds of the invention comprise a 5-membered or 6-membered monocyclic heteroaryl group comprising at least one nitrogen ring atom, e.g., such groups as particularly disclosed herein. Selected 5-membered heteroaryl groups contain one nitrogen, and optionally contain one oxygen ring atom or 1, 2 or 3 additional nitrogen ring atoms. Selected 6-membered heteroaryl groups contain 1, 2, or 3 nitrogen ring heteroatoms.

In other embodiments, compounds of the invention comprise a 9-membered or 10-membered bicyclic heteroaryl group, e.g. such groups as particularly disclosed herein. Selected 9-10 membered heteroaryl groups contain one nitrogen, oxygen or sulfur ring heteroatom, and optionally contain 1, 2, or 3 additional nitrogen ring atoms.

It is to be understood that the terms heterocyclic, heteroaryl, and heterocycloalkyl are intended to encompass stable heterocyclic groups where a ring nitrogen heteroatom is optionally oxidized (e.g., heterocyclic groups containing an N-oxide, e.g., pyridine-N-oxide), or where a ring sulfur heteroatom is optionally oxidized (e.g., heterocyclic groups containing sulfones or sulfoxide moieties, e.g., tetrahydrothienyl-1-oxide [a tetrahydrothienyl sulfoxide], tetrahydrothienyl-1,1-dioxide [a tetrahydrothienyl sulfone], or 1,1-dioxido-2,3-dihydrobenzoisothiazolyl [e.g., 1,1-dioxido-2,3-dihydrobenzo[d]isothiazolyl]).

When a particular heterocyclic, heterocycloalkyl or heteroaryl group is referenced (e.g., pyridyl), it is intended to encompass any one of the various position isomers (e.g., 2-pyridyl, 3-pyridyl, etc). Unless otherwise indicated, carbocyclic and heterocyclic ring moieties may be linked to the remaining molecule through any of their ring atoms, including for example a ring nitrogen atom.

When the term “alkyl” is used in combination with other groups, e.g., “(C₁-C₃)alkyl-(C₃-C₆)cycloalkyl”, “(C₁-C₃)alkyl-phenyl” and “(C₁-C₃)alkyl-heterocyclyl”, the alkyl moiety is intended to encompass a divalent straight or branched-chain hydrocarbon radical and the cycloalkyl, phenyl, and heterocyclyl moieties are as defined herein. For example, in “(C₁-C₃)alkyl-phenyl” the (C₁-C₃)alkyl moiety thereof is a divalent straight or branched-chain carbon radical linked to the aryl group phenyl, and is represented by the bonding arrangement present in a benzyl group (—CH₂-phenyl). Particular examples of such groups include (cyclopentyl)methyl, 2-phenylethyl, 3-phenylpropyl, and 2-naphthylethyl.

“Oxo” represents a double-bonded oxygen moiety; for example, if attached directly to a carbon atom forms a carbonyl moiety (C═O). The terms “halogen” and “halo” represent chloro, fluoro, bromo or iodo substituents. “Hydroxy” or “hydroxyl” is intended to mean the radical —OH. “Cyano” means the radical —CN. “Nitro” means the radical —NO₂. “COO” and “CO₂” may be used interchangeably herein (e.g., COOH and CO₂H; COOEt and CO₂Et are interchangeable respectively). PO₃H₂ and P(O)(OH)₂ may be used interchangeably herein.

As used herein, “amino acid side chain” is a side chain derived from a D- or L-form of alanine, arginine, asparagine, aspartic acid, cysteine, cystine, glutamic acid, glutamine, glycine, histidine, hydroxylysine, hydroxyproline, isoleucine, leucine, lysine, methionine, phenylalanine, proline, serine, threonine, tryptophan, tyrosine, or valine.

As used herein, “tripeptide” is a peptide formed from 3 amino acids selected from D- and/or L-forms of alanine, arginine, asparagine, aspartic acid, cysteine, cystine, glutamic acid, glutamine, glycine, histidine, hydroxylysine, hydroxyproline, isoleucine, leucine, lysine, methionine, phenylalanine, proline, serine, threonine, tryptophan, tyrosine, and/or valine. In some embodiments, the tripeptide is (L-Lysine-L-Leucine-D-Valine) or (L-Lysine-L-Phenylalanine-D-Alanine).

Where a numerical range is indicated, e.g., a carbon number range or a heteroatom number range, the range is intended to encompass particular embodiments corresponding to the particular integers within the range, and well as any range of integers within the most broadly stated range.

As used herein, the terms “compound(s) of the invention” or “compound(s) of this invention” mean a compound of Formula (I) or (II), as defined above (including more particular embodiments), in any form, i.e., any salt or non-salt form (e.g., as a free acid or base form, or as a salt, particularly a pharmaceutically acceptable salt thereof) and any physical form thereof (e.g., including non-solid forms (e.g., liquid or semi-solid forms), and solid forms (e.g., amorphous or crystalline forms, specific polymorphic forms, solvate forms, including hydrate forms (e.g., mono-, di- and hemi-hydrates)), and mixtures of various forms.

Accordingly, included within the present invention are compounds of Formulas (I) and (II), as defined herein (including more particular embodiments), in any salt or non-salt form and any physical form thereof, and mixtures of various forms. While such are included within the present invention, it will be understood that the compounds of Formulas (I) and (II), as defined herein, in any salt or non-salt form, and in any physical form thereof, may have varying levels of activity, different bioavailabilities and different handling properties for formulation purposes.

As used herein, the term “optionally substituted” indicates that a group, ring or moiety (such as an alkyl, cycloalkyl, alkoxy, cycloalkoxy, heterocycloalkyl, phenyl, heteroaryl, carbocyclic or heterocyclic group, ring or moiety) may be unsubstituted, or the group, ring or moiety may be substituted with one or more substituent(s) as defined. In the case where more than one group, ring or moiety may be or are substituted with a number of alternative substituent(s), the selected substituent(s) for each group, ring or moiety may be the same or different, i.e. the substituent(s) are selected independently for each group, ring or moiety. In the case where more than one substituent is selected from a number of possible substituents, those substituents may be the same or different, i.e. the substituents are selected independently.

As used herein, the terms “a”, “an” and “the” are intended to include one or more of the indicated moiety, unless otherwise indicated.

As used herein, “at least one” includes one and other particular higher quantities permitted in light of the disclosure, unless otherwise stated. For example, where a group may be substituted by 1-3 substituents, when the group is substituted by “at least one” substituent, it may be independently substituted by one, two or three substituents.

As used herein, “BMP1, TLL1 and/or TLL2” encompasses one or more of BMP1, TLL1 and TLL2, including isoforms thereof (including particularly isoforms encoded by RNA splice variants). Thus, for example, as used herein BMP1 may include one or more of the isoforms BMP-1-1, BMP-1-2, BMP-1-3, BMP-1-4, BMP-1-5, BMP-1-6, and BMP-1-7.

All references/publications are hereby incorporated by reference into this disclosure in their entirety.

In one aspect, the present invention is directed to a compound of Formula (I) as defined above, or a salt thereof.

In another aspect, the present invention is directed to a compound of Formula (II) as defined above, or a salt thereof.

### Compounds of Formula (I)

In some embodiments, the compound according to Formula (I) has the Formula (I)(a):

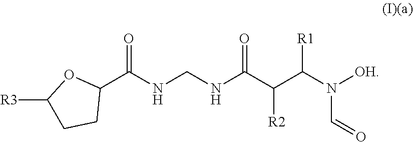

In other embodiments, the compound according to Formula (I) has the Formula (I)(b):

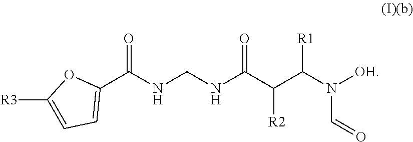

In some embodiments of the compounds of Formula (I), R1 is (C₁-C₄) straight chain alkyl. In some embodiments R1 is H, methyl, ethyl, or —CH₂OH; in more particular embodiments H, ethyl or —CH₂OH, more particularly H or ethyl and especially ethyl. In some embodiments, R1 is (C₁-C₄) straight chain alkyl substituted with one hydroxy group.

In some embodiments of the compounds of Formula (I), R2 is (C₁-C₁₁) alkyl. In some embodiments, R2 is H, n-pentyl, 2-ethylbutyl, (cyclopentyl)methyl, benzyl, 2-phenylethyl, or 3-phenylpropyl (in more particular embodiments n-pentyl, (cyclopentyl)methyl, 2-phenylethyl, or 3-phenylpropyl, even more particularly n-pentyl), where such groups are optionally substituted as defined above in accordance with Formula (I). In some embodiments of the compounds of Formula (I), R2 is 2-naphthylethyl, optionally substituted as defined above in accordance with Formula (I). In some embodiments such groups are unsubstituted. In some embodiments, R2 is n-pentyl.

In some embodiments, R1 is H, and R2 has (R) stereochemistry. In other embodiments, R1 is —CH₂OH and has (S) stereochemistry, and R2 has (R) stereochemistry. In other embodiments, R1 is (C₁-C₄) straight chain alkyl or (C₂-C₄) straight chain alkyl substituted with one hydroxy group, and both R1 and R2 have (R) stereochemistry.

In some embodiments, R′ is independently selected from phenyl, (5-methyl-2-oxo-1,3-dioxol-4-yl)methyl, and (C₁-C₄)alkyl, wherein said (C₁-C₄)alkyl is optionally substituted as defined in reference to Formula (I). In some embodiments, at least one of the R3 phenyl or heteroaryl substituents comprise at least one —CO₂R′ group, wherein R′ is phenyl, (5-methyl-2-oxo-1,3-dioxol-4-yl)methyl or (C₁-C₄)alkyl, wherein said (C₁-C₄)alkyl is optionally substituted as defined in reference to Formula (I).

In some embodiments, R″ is independently selected from (C₁-C₄)alkyl; (C₁-C₄)alkoxy; benzyloxy; phenoxy; (5-methyl-2-oxo-1,3-dioxol-4-yl)methoxy; and —OCR^{a5}R^{b5}OC(O)R^{z}, wherein R^{a5}, R^{b5} and R^{z} are independently selected from the groups as defined in reference to Formula (I). In some embodiments, at least one of the R3 phenyl or heteroaryl substituents comprise at least one —P(O)R″R″ or —OP(O)R″R″ group, wherein one or both R″ is independently selected from phenoxy, benzyloxy, (5-methyl-2-oxo-1,3-dioxol-4-yl)methoxy, and —OCR^{a5}R^{b5}OC(O)R^{z} wherein R^{a5}, R^{b5} and R^{z} are independently selected from the groups as defined in reference to Formula (I).

In some embodiments, R′ is independently selected from (C₁-C₄)alkyl and benzyl and R″ is independently selected from the group consisting of: (C₁-C₄)alkyl; (C₁-C₄)alkoxy; benzyloxy; and —OCR^{a5}R^{b5}OC(O)R^{z}.

In some embodiments, at least one of the R3 phenyl or heteroaryl substituents comprise at least one —CO₂R′ group, wherein R′ is (C₁-C₄)alkyl or benzyl.

In some embodiments, at least one of the R3 phenyl or heteroaryl substituents comprise at least one —P(O)R″R″ or —OP(O)R″R″ group, wherein one or both R″ of said —P(O)R″R″ or —OP(O)R″R″ is independently selected from C₃ and C₄ alkyl.

In some embodiments, at least one of the R3 phenyl or heteroaryl substituents comprise at least one —P(O)R″R″ or —OP(O)R″R″ group, wherein one or both R″ of said —P(O)R″R″ or —OP(O)R″R″ is independently selected from said benzyloxy and —OCR^{a5}R^{b5}OC(O)R^{z}.

In some embodiments of the compounds of Formula (I), R3 is phenyl, pyridyl, pyridazinyl, pyrimidinyl, oxazolyl, tetrazolyl, pyrazolyl, indazolyl, or 1,1-dioxido-2,3-dihydrobenzo[d]isothiazolyl (in more particular embodiments, phenyl, pyridyl, indazolyl, or 1,1-dioxido-2,3-dihydrobenzo[d]isothiazolyl), including the various position isomers thereof, where such groups are optionally substituted as defined above in accordance with Formula (I), including more particular embodiments of Formula (I).

In more particular embodiments, R3 is phenyl optionally substituted in accordance with the definition of Formula (I), including more particular embodiments of Formula (I).

In more particular embodiments, R3 is 3,4- or 3,5-disubstituted phenyl wherein the substituent groups are selected in accordance with the definition of Formula (I), including more particular embodiments of Formula (I) (said positions relative to the point of attachment of the phenyl ring to the remainder of the compound of Formula (I)).

In some embodiments, R3 is phenyl substituted with ethoxy in the 3-position and —P(O)R″R″ in the 4- or 5-position (more particularly in the 5-position), in which one or both R″ is independently selected from (C₁-C₄)alkoxy; benzyloxy; phenoxy; (5-methyl-2-oxo-1,3-dioxol-4-yl)methoxy; and said —OCR^{a5}R^{b5}OC(O)R^{z} (wherein R^{a5}, R^{b5} and R^{z} are independently selected from the groups as defined in reference to Formula (I)). It will be understood that where only one of said R″ groups is so selected, the other R″ may be selected from any of those groups defined for formula (I). In particular embodiments, the R″ groups are independently selected from —OH, phenoxy, —OCR^{a5}R^{b5}OC(O)R^{z} (wherein R^{a5} and R^{b5} are independently selected from H and (C₁-C₄)alkyl) and R^{z} is selected from (C₁-C₅)alkyl and (C₁-C₅)alkoxy), and (5-methyl-2-oxo-1,3-dioxol-4-yl)methoxy, provided that at least one R″ must be other than —OH. In some of the foregoing embodiments the R″ groups are the same; in others one R″ is OH and the other is selected from the aforementioned groups. In some embodiments said —OCR^{a5}R^{b5}OC(O)R^{z} is —OCH₂OC(O)R^{z}.

In some embodiments, R3 is phenyl substituted with ethoxy in the 3-position, and —C(O)NHCH(CO₂R′)(CH₂CO₂R′) in the 4- or 5-position (more particularly in the 4-position), in which one or both R′ is independently benzyl, phenyl, or (C₁-C₄)alkyl, wherein said alkyl is optionally substituted with 1-2 groups independently selected from —OC(O)R^{g} wherein R^{g} is (C₁-C₄)alkyl or phenyl, and (5-methyl-2-oxo-1,3-dioxol-4-yl)methoxy. It will be understood that where only one of said R′ groups is so selected, the other R′ may be selected from any of those groups defined for formula (I). In particular embodiments, the R′ groups are independently selected from —H, phenyl and (C₁-C₄)alkyl optionally substituted as stated above, provided that at least one R′ must be other than H.

In some embodiments, R3 is phenyl substituted with —OCH₂CO₂R′ in the 3-position and —C(O)NHCH(CO₂R′)(CH₂CO₂R′) in the 4 or 5 position (more particularly in the 4-position), in which R′ of —OCH₂CO₂R′, and/or one or both R′ of —C(O)NHCH(CO₂R′)(CH₂CO₂R′) is independently selected from (C₁-C₄)alkyl and benzyl.

In some embodiments, R3 is phenyl substituted with —OCH₂CO₂CH₂phenyl in the 3-position and —C(O)NHCH(CO₂CH₂phenyl)(CH₂CO₂CH₂phenyl) in the 4-position; or —OCH₂CO₂CH₃ in the 3-position and —C(O)NHCH(CO₂CH₃)(CH₂CO₂CH₃) in the 4-position.

In other embodiments, R3 is phenyl substituted with one —CO₂R′ group, wherein R′ is phenyl or (C₁-C₄)alkyl, wherein said (C₁-C₄)alkyl is optionally substituted with 1-2 groups independently selected from phenyl, heteroaryl, NR^{f}R^{f} wherein each R^{f} is independently selected from H and (C₁-C₄)alkyl, heterocycloalkyl, —OC(O)O(C₁-C₄)alkyl, —OC(O)R^{g} wherein R^{g} is (C₁-C₄)alkyl or phenyl, and —C(O)NR^{h}R^{h} wherein R^{h} is independently selected from H and (C₁-C₄)alkyl. In more particular embodiments R′ of —CO₂R′ is (C₁-C₄)alkyl, optionally substituted with 1-2 groups selected from phenyl, NR^{f}R^{f} wherein each R^{f} is independently selected from H and (C₁-C₄)alkyl, heterocycloalkyl, and —C(O)NR^{h}R^{h} wherein R^{h} is independently selected from H and (C₁-C₄)alkyl; in more particular embodiments said R′ is phenethyl.

Other particular examples of R3 phenyl substituents include: —OCH₃, —OC₂H₅, —OC₃H₇, —OCH(CH₃)₂, —OCF₃, —OCHF₂, —OCH₂CF₃, —OCH₂CHF₂, —OC₂H₄-pyrrolidine, —OCH₂CO₂H, —OCH₂C(O)NH₂, —CO₂H, —CH₃, cyclopropane-1-carboxylic acid, —CH₂CO₂H, —C(CH₃)₂CO₂H, —CH(CH₃)CO₂H, —CF₂CO₂H, —CH₂C(O)NHCH(CO₂H)(CH₂CO₂H), —CH₂P(O)(OH)₂, —CH₂N(CH₃)(CH₂CO₂H), —CH₂NHCH₂P(O)(OH)₂, —C(NH₂)(NOH), cyano, nitro, hydroxy, —SO₂NH₂, —SO₂N(CH₃)₂, —SO₂NH(CH₃), —SO₂CH₃, —SO₂NHC(O)C₂H₅, —SCH₃, —SC₂H₅, —C(O)OCH₃, —C(O)OC(CH₃)₃, —C(O)NHCH₃, —C(O)NH(C₂H₄NH₂), —C(O)NHC₂H₄N⁺(CH₃)₃, —C(O)NHCH(CO₂H)(CH₂CO₂H), —C(O)NHCH(CO₂H)(C₂H₄CO₂H), —C(O)NHCH₂CO₂H, —C(O)N(CH₂CO₂H)₂, —C(O)NHCH₂P(O)(OH)₂, —C(O)NHC(CH₂OH)₃, fluoro, —NH₂, —N⁺(CH₃)₂, —P(O)(CH₃)(OC₂H₅), —P(O)(OCH₃)₂, —P(O)(CH₃)(OH), —P(O)(OH)(OCH₃), and —P(O)(OH)₂. In some embodiments, other particular R3 phenyl substitutents are selected from: —OC₂H₅, hydroxy, —CO₂H, —OCH₂CO₂H, —P(O)(OH)₂, —C(O)NHCH(CO₂H)(CH₂CO₂H) and —C(O)NHCH₂P(O)(OH)₂, or a combination thereof.

In some embodiments of compounds of Formula (I), R3 is phenyl substituted with 1-2 groups selected from:

### OEt,

### OCH₂COOMe,

### COOEt,

### COObutyl,

### COOEtPh,

COOEtmorpholinyl (especially N-linked),

### COOEtN(Me)₂,

COOCH₂CON(Me)₂,

### CONHCH(CH₂COOH)(COOEt),

CONHCH(CH₂COOH)(COOC(Me)₃),

### CONHCH(CH₂COOH)(COOPh),

CONHCH(CH₂COOH)(COOCH₂-[5-Me-2-oxo-1,3-dioxol-4yl]),
CONHCH(CH₂COOH)(COOCH₂OC(O)iPr),
CONHCH(CH₂COOH)(COOCH₂OC(O)Ph),

### CONHCH(CH₂COOMe)(COOMe),

### CONHCH(CH₂COOEt)(COOEt),

CONHCH(CH₂COOC(Me)₃)(COOC(Me)₃),
CONHCH(CH₂COOCH₂OC(O)iPr)(COOCH₂OC(O)iPr),

### PO(OH)(OPh),

### PO(OH)(OCH₂OC(O)iPr),

PO(OH)(OCH₂-[5-Me-2-oxo-1,3-dioxol-4yl]),

### PO(OH)(OCH₂OC(O)OiPr),

### PO(OPh)₂,

PO(OCH₂OC(O)Me)₂,
PO(OCH₂OC(O)iPr)₂, and
PO(OCH₂OC(O)OiPr)₂.
(in some more particular embodiments where a substituent includes a heterocyclic ring having a ring N atom, the ring is N-linked to the rest of the molecule). In some of the foregoing embodiments, one group is OEt and the other is selected from the above stated group.

In each of the aforementioned particular embodiments of R3 phenyl and R3 substituted phenyl, in some embodiments R1 is (C₁-C₄) straight chain alkyl (especially ethyl), and R2 is (C₁-C₁₁) alkyl (especially pentyl).

In some embodiments of the compounds of Formula (I), R3 is optionally substituted heteroaryl, e.g.: pyridyl, pyridazinyl, pyrimidinyl, oxazolyl, tetrazolyl, pyrazolyl, indazolyl, or 1,1-dioxido-2,3-dihydrobenzo[d]isothiazolyl. In some embodiments, R3 is optionally substituted pyridyl, indazolyl, or 1,1-dioxido-2,3-dihydrobenzo[d]isothiazolyl (including particularly pyridin-3-yl, pyridin-2-yl, 1,1-dioxido-2,3-dihydrobenzo[d]isothiazol-6-yl, and indazol-6-yl). In these embodiments such R3 groups may be optionally substituted as defined in accordance with Formula (I), including more particular embodiments of Formula (I).

In some embodiments, the heteroaryl or phenyl R3 group is substituted with 1-3 (e.g. 1-2) groups independently selected from: ethoxy; P(O)R″R″; —OCH₂CO₂R′; and —C(O)NHCH(CO₂R′)(CH₂CO₂R′); where at least one R′ is (C₁-C₄)alkyl or benzyl; and at least one R″ is selected from (C₁-C₄)alkoxy, benzyloxy, and said —OCR^{a5}R^{b5}OC(O)R^{z}.

In some embodiments, the heteroaryl or phenyl R3 group is substituted with ethoxy and P(O)R″R″ or —C(O)NHCH(CO₂R′)(CH₂CO₂R′); or —OCH₂CO₂R′ and —C(O)NHCH(CO₂R′)(CH₂CO₂R′); where at least one R′ of the respective groups is (C₁-C₄) alkyl or benzyl (one or both R′ may be such groups, as applicable); and at least one R″ is selected from (C₁-C₄)alkoxy, benzyloxy, and said —OCR^{a5}R^{b5}OC(O)R^{z} (one or both may be such groups).

It is to be understood that in relation to particular embodiments of Formula (I), when one R′ or R″ group in a moiety is particularly defined and the other(s) is not, the other(s) may be respectively selected from the group defined for R′ or R″ according to Formula (I).

In some embodiments, all R′ groups in a particular moiety and/or in the compound of Formula (I) are the same. In some embodiments, all R″ groups in a particular moiety and/or in the compound of Formula (I) are the same. In some embodiments, the R′ groups in a particular moiety and/or in the compound of Formula (I) may be different. In some embodiments, the R″ groups in a particular moiety and/or in the compound of Formula (I) may be different. In some embodiments, all the R′ and R″ groups in a particular moiety and/or the compound of Formula (I) are the same. In some embodiments, the R′ and R″ groups in a particular moiety and/or the compound of Formula (I) may be different.

In some embodiments, R3 heteroaryl is substituted with —OCH₂CO₂CH₂phenyl and —C(O)NHCH(CO₂CH₂phenyl)(CH₂CO₂CH₂phenyl); or —OCH₂CO₂CH₃ and —C(O)NHCH(CO₂CH₃)(CH₂CO₂CH₃).

Other particular examples of R3 heteroaryl substituents include: —OCH₃, —OC₂H₅, —OC₃H₇, —OCH(CH₃)₂, —OCF₃, —OCHF₂, —OCH₂CF₃, —OCH₂CHF₂, —OC₂H₄-pyrrolidine, —OCH₂CO₂H, —OCH₂C(O)NH₂, —CO₂H, —CH₃, cyclopropane-1-carboxylic acid, —CH₂CO₂H, —C(CH₃)₂CO₂H, —CH(CH₃)CO₂H, —CF₂CO₂H, —CH₂C(O)NHCH(CO₂H)(CH₂CO₂H), —CH₂P(O)(OH)₂, —CH₂N(CH₃)(CH₂CO₂H), —CH₂NHCH₂P(O)(OH)₂, —C(NH₂)(NOH), cyano, nitro, hydroxy, —SO₂NH₂, —SO₂N(CH₃)₂, —SO₂NH(CH₃), —SO₂CH₃, —SO₂NHC(O)C₂H₅, —SCH₃, —SC₂H₅, —C(O)OCH₃, —C(O)OC(CH₃)₃, —C(O)NHCH₃, —C(O)NH(C₂H₄NH₂), —C(O)NHC₂H₄N⁺(CH₃)₃, —C(O)NHCH(CO₂H)(CH₂CO₂H), —C(O)NHCH(CO₂H)(C₂H₄CO₂H), —C(O)NHCH₂CO₂H, —C(O)N(CH₂CO₂H)₂, —C(O)NHCH₂P(O)(OH)₂, —C(O)NHC(CH₂OH)₃, fluoro, —NH₂, —N(CH₃)₂, —P(O)(CH₃)(OC₂H₅), —P(O)(OCH₃)₂, —P(O)(CH₃)(OH), —P(O)(OH)(OCH₃), and —P(O)(OH)₂. In some embodiments, other R3 heteroaryl substituents are independently selected from: —OCH₃, —OC₂H₅, —OC₃H₇, —OCH(CH₃)₂, —CO₂H, —CH₃, —P(O)(CH₃)(OC₂H₅), —P(O)(OCH₃)₂, —P(O)(CH₃)(OH), —P(O)(OH)(OCH₃), and —P(O)(OH)₂, or a combination thereof; more particularly —OCH₃, —CH₃, and —CO₂H or a combination thereof.

In some embodiments, Formula (I) herein does not encompass a compound disclosed in PCT/IB2015/050179 or PCT publication no. WO2015/104684. In some embodiments, the compound of Formula (I) herein is not a compound disclosed in PCT/IB2015/050179 or PCT publication no. WO2015/104684, for example, the compound of Formula (I) is not the title compound of WO2015/104684 Examples 16, 26, 31, 36, 45, 47, 48, 50, 51, 57, 58, 63, 95, or 101. PCT/IB2015/050179 and PCT publication no. WO2015/104684 is incorporated herein by reference, including for the purpose of defining embodiments of the present invention which exclude any subject matter disclosed therein.

Accordingly, a compound of the invention may be a compound of Formula (I), or a salt thereof, particularly a pharmaceutically acceptable salt thereof. Representative compounds of the invention include the specific compounds described herein, e.g., the compounds of Formula (I) of the Examples, as well as any alternative stereoisomeric forms, free acid/base forms, salt forms, and alternative salt forms thereof (particularly pharmaceutically acceptable salt or alternative salt forms thereof), as applicable. Accordingly, in some embodiments the compound of the invention is a compound of Formula (I) selected from:

dimethyl 2-(4-(5-(((2-(1-(N-
hydroxyformamido)propyl)heptanamido)methyl)carbamoyl)furan-2-
yl)-2-(2-methoxy-2-oxoethoxy)benzamido)succinate
ethyl 4-(5-(((2-(1-(N-
hydroxyformamido)propyl)heptanamido)methyl)carbamoyl)furan-2-
yl)benzoate
butyl 4-(5-(((2-(1-(N-
hydroxyformamido)propyl)heptanamido)methyl)carbamoyl)furan-2-
yl)benzoate
phenethyl 4-(5-(((2-(1-(N-
hydroxyformamido)propyl)heptanamido)methyl)carbamoyl)furan-2-
yl)benzoate
2-morpholinoethyl 4-(5-(((2-(1-(N-
hydroxyformamido)propyl)heptanamido)methyl)carbamoyl)furan-2-
yl)benzoate
2-(dimethylamino)ethyl 4-(5-(((2-(1-(N-
hydroxyformamido)propyl)heptanamido)methyl)carbamoyl)furan-2-
yl)benzoate
2-(dimethylamino)-2-oxoethyl 4-(5-(((2-(1-(N-
hydroxyformamido)propyl)heptanamido)methyl)carbamoyl)furan-2-
yl)benzoate
3-(2-ethoxy-4-(5-(((2-(1-(N-
hydroxyformamido)propyl)heptanamido)methyl)carbamoyl)furan-2-
yl)benzamido)-4-((isobutyryloxy)methoxy)-4-oxobutanoic acid
(((3-ethoxy-5-(5-(((2-(1-(N-
hydroxyformamido)propyl)heptanamido)methyl)carbamoyl)furan-2-
yl)phenyl)phosphoryl)bis(oxy))bis(methylene) diisopropyl dicarbonate
diphenyl (3-ethoxy-5-(5-(((2-(1-(N-
hydroxyformamido)propyl)heptanamido)methyl)carbamoyl)furan-2-
yl)phenyl)phosphonate
(((3-ethoxy-5-(5-(((2-(1-(N-
hydroxyformamido)propyl)heptanamido)methyl)carbamoyl)furan-2-
yl)phenyl)phosphoryl)bis(oxy))bis(methylene) bis(2-
methylpropanoate)
3-(2-ethoxy-4-(5-(((2-(1-(N-
hydroxyformamido)propyl)heptanamido)methyl)carbamoyl)furan-2-
yl)benzamido)-4-oxo-4-phenoxybutanoic acid
bis((isobutyryloxy)methyl) 2-(2-ethoxy-4-(5-(((2-(1-(N-
hydroxyformamido)propyl)heptanamido)methyl)carbamoyl)furan-2-
yl)benzamido)succinate
di-tert-butyl 2-(2-ethoxy-4-(5-(((2-(1-(N-
hydroxyformamido)propyl)heptanamido)methyl)carbamoyl)furan-2-
yl)benzamido)succinate
4-ethoxy-3-(2-ethoxy-4-(5-(((2-(1-(N-
hydroxyformamido)propyl)heptanamido)methyl)carbamoyl)furan-2-
yl)benzamido)-4-oxobutanoic acid
4-(tert-butoxy)-3-(2-ethoxy-4-(5-(((2-(1-(N-
hydroxyformamido)propyl)heptanamido)methyl)carbamoyl)furan-2-
yl)benzamido)-4-oxobutanoic acid
(((3-ethoxy-5-(5-(((2-(1-(N-
hydroxyformamido)propyl)heptanamido)methyl)carbamoyl)furan-2-
yl)phenyl)phosphoryl)bis(oxy))bis(methylene) diacetate
4-((benzoyloxy)methoxy)-3-(2-ethoxy-4-(5-(((2-(1-(N-
hydroxyformamido)propyl)heptanamido)methyl)carbamoyl)furan-2-
yl)benzamido)-4-oxobutanoic acid
diethyl 2-(2-ethoxy-4-(5-(((2-(1-(N-
hydroxyformamido)propyl)heptanamido)methyl)carbamoyl)furan-2-
yl)benzamido)succinate
3-(2-ethoxy-4-(5-(((2-(1-(N-
hydroxyformamido)propyl)heptanamido)methyl)carbamoyl)furan-2-
yl)benzamido)-4-((5-methyl-2-oxo-1,3-dioxol-4-yl)methoxy)-4-
oxobutanoicacid
(((3-ethoxy-5-(5-(((2-(1-(N-
hydroxyformamido)propyl)heptanamido)methyl)carbamoyl)furan-2-
yl)phenyl)(hydroxy)phosphoryl)oxy)methyl isobutyrate
(5-methyl-2-oxo-1,3-dioxol-4-yl)methyl hydrogen (3-ethoxy-5-(5-(((2-
(1-(N-
hydroxyformamido)propyl)heptanamido)methyl)carbamoyl)furan-2-
yl)phenyl)phosphonate
(((3-ethoxy-5-(5-(((2-(1-(N-
hydroxyformamido)propyl)heptanamido)methyl)carbamoyl)furan-2-
yl)phenyl)(hydroxy)phosphoryl)oxy)methyl isopropyl carbonate; and
phenyl hydrogen (3-ethoxy-5-(5-(((2-(1-(N-
hydroxyformamido)propyl)heptanamido)methyl)carbamoyl)furan-2-
yl)phenyl)phosphonate

or a salt thereof (in more particular embodiments, a pharmaceutically acceptable salt thereof).

In some embodiments the compound of the invention is a compound of Formula (I) selected from:

(S)-dimethyl 2-(4-(5-((((R)-2-((R)-1-(N-
hydroxyformamido)propyl)heptanamido)methyl)carbamoyl)furan-2-
yl)-2-(2-methoxy-2-oxoethoxy)benzamido)succinate
ethyl 4-(5-((((R)-2-((R)-1-(N-
hydroxyformamido)propyl)heptanamido)methyl)carbamoyl)furan-2-
yl)benzoate
butyl 4-(5-((((R)-2-((R)-1-(N-
hydroxyformamido)propyl)heptanamido)methyl)carbamoyl)furan-2-
yl)benzoate
phenethyl 4-(5-((((R)-2-((R)-1-(N-
hydroxyformamido)propyl)heptanamido)methyl)carbamoyl)furan-2-
yl)benzoate
2-morpholinoethyl 4-(5-((((R)-2-((R)-1-(N-
hydroxyformamido)propyl)heptanamido)methyl)carbamoyl)furan-2-
yl)benzoate
2-(dimethylamino)ethyl 4-(5-((((R)-2-((R)-1-(N-
hydroxyformamido)propyl)heptanamido)methyl)carbamoyl)furan-2-
yl)benzoate
2-(dimethylamino)-2-oxoethyl 4-(5-((((R)-2-((R)-1-(N-
hydroxyformamido)propyl)heptanamido)methyl)carbamoyl)furan-2-
yl)benzoate
(S)-3-(2-ethoxy-4-(5-((((R)-2-((R)-1-(N-
hydroxyformamido)propyl)heptanamido)methyl)carbamoyl)furan-2-
yl)benzamido)-4-((isobutyryloxy)methoxy)-4-oxobutanoic acid
(((3-ethoxy-5-(5-((((R)-2-((R)-1-(N-
hydroxyformamido)propyl)heptanamido)methyl)carbamoyl)furan-2-
yl)phenyl)phosphoryl)bis(oxy))bis(methylene) diisopropyl dicarbonate
diphenyl (3-ethoxy-5-(5-((((R)-2-((R)-1-(N-
hydroxyformamido)propyl)heptanamido)methyl)carbamoyl)furan-2-
yl)phenyl)phosphonate
(((3-ethoxy-5-(5-((((R)-2-((R)-1-(N-
hydroxyformamido)propyl)heptanamido)methyl)carbamoyl)furan-2-
yl)phenyl)phosphoryl)bis(oxy))bis(methylene) bis(2-
methylpropanoate)
(S)-3-(2-ethoxy-4-(5-((((R)-2-((R)-1-(N-
hydroxyformamido)propyl)heptanamido)methyl)carbamoyl)furan-2-
yl)benzamido)-4-oxo-4-phenoxybutanoic acid
(S)-bis((isobutyryloxy)methyl) 2-(2-ethoxy-4-(5-((((R)-2-((R)-1-(N-
hydroxyformamido)propyl)heptanamido)methyl)carbamoyl)furan-2-
yl)benzamido)succinate
(S)-di-tert-butyl 2-(2-ethoxy-4-(5-((((R)-2-((R)-1-(N-
hydroxyformamido)propyl)heptanamido)methyl)carbamoyl)furan-2-
yl)benzamido)succinate
(S)-4-ethoxy-3-(2-ethoxy-4-(5-((((R)-2-((R)-1-(N-
hydroxyformamido)propyl)heptanamido)methyl)carbamoyl)furan-2-
yl)benzamido)-4-oxobutanoic acid
(S)-4-(tert-butoxy)-3-(2-ethoxy-4-(5-((((R)-2-((R)-1-(N-
hydroxyformamido)propyl)heptanamido)methyl)carbamoyl)furan-2-
yl)benzamido)-4-oxobutanoic acid
(((3-ethoxy-5-(5-((((R)-2-((R)-1-(N-
hydroxyformamido)propyl)heptanamido)methyl)carbamoyl)furan-2-
yl)phenyl)phosphoryl)bis(oxy))bis(methylene) diacetate
(S)-4-((benzoyloxy)methoxy)-3-(2-ethoxy-4-(5-((((R)-2-((R)-1-(N-
hydroxyformamido)propyl)heptanamido)methyl)carbamoyl)furan-2-
yl)benzamido)-4-oxobutanoic acid
(S)-diethyl 2-(2-ethoxy-4-(5-((((R)-2-((R)-1-(N-
hydroxyformamido)propyl)heptanamido)methyl)carbamoyl)furan-2-
yl)benzamido)succinate
(S)-3-(2-ethoxy-4-(5-((((R)-2-((R)-1-(N-
hydroxyformamido)propyl)heptanamido)methyl)carbamoyl)furan-2-
yl)benzamido)-4-((5-methyl-2-oxo-1,3-dioxol-4-yl)methoxy)-4-
oxobutanoicacid
(((3-ethoxy-5-(5-((((R)-2-((R)-1-(N-
hydroxyformamido)propyl)heptanamido)methyl)carbamoyl)furan-2-
yl)phenyl)(hydroxy)phosphoryl)oxy)methyl isobutyrate
(5-methyl-2-oxo-1,3-dioxol-4-yl)methyl hydrogen (3-ethoxy-5-(5-
((((R)-2-((R)-1-(N-
hydroxyformamido)propyl)heptanamido)methyl)carbamoyl)furan-2-
yl)phenyl)phosphonate
(((3-ethoxy-5-(5-((((R)-2-((R)-1-(N-
hydroxyformamido)propyl)heptanamido)methyl)carbamoyl)furan-2-
yl)phenyl)(hydroxy)phosphoryl)oxy)methyl isopropyl carbonate; and
phenyl hydrogen (3-ethoxy-5-(5-((((R)-2-((R)-1-(N-
hydroxyformamido)propyl)heptanamido)methyl)carbamoyl)furan-2-
yl)phenyl)phosphonate

or a salt thereof (in more particular embodiments, a pharmaceutically acceptable salt thereof).

### Compounds of Formula (II)

In some embodiments, the compound according to Formula (II) has the Formula (II)(a):

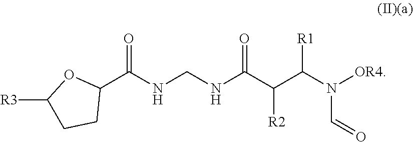

In other embodiments, the compound according to Formula (II) has the Formula (II)(b):

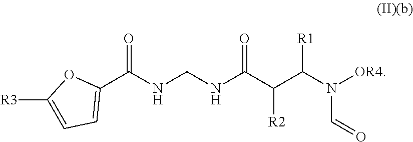

In some embodiments of the compounds of Formula (II), R1 is (C₁-C₄) straight chain alkyl. In some embodiments, R1 is H, methyl, ethyl, or —CH₂OH; in more particular embodiments H, ethyl or —CH₂OH, more particularly H or ethyl and especially ethyl. In some embodiments, R1 is (C₁-C₄) straight chain alkyl substituted with one hydroxy group.

In some embodiments of the compounds of Formula (II), R2 is (C₁-C₁₁) alkyl. In some embodiments, R2 is H, n-pentyl, 2-ethylbutyl, (cyclopentyl)methyl, benzyl, 2-phenylethyl, or 3-phenylpropyl (in more particular embodiments n-pentyl, (cyclopentyl)methyl, 2-phenylethyl, or 3-phenylpropyl, even more particularly n-pentyl), where such groups are optionally substituted as defined above in accordance with Formula (II). In some embodiments of the compounds of Formula (II), R2 is 2-naphthylethyl, optionally substituted as defined above in accordance with Formula (II). In some embodiments such groups are unsubstituted. In some embodiments, R2 is n-pentyl.

In some embodiments, R1 is H, and R2 has (R) stereochemistry. In other embodiments, R1 is —CH₂OH and has (S) stereochemistry, and R2 has (R) stereochemistry. In other embodiments, R1 is (C₁-C₄) straight chain alkyl or (C₂-C₄) straight chain alkyl substituted with one hydroxy group, and both R1 and R2 have (R) stereochemistry.

In some embodiments of the compounds of Formula (II), at least one of said R3 phenyl and heteroaryl optional substituents comprises at least 1 group selected from:

—CO₂R′, wherein R′ is (C₁-C₄)alkyl or benzyl;
—P(O)R″R″, wherein one R″ is selected from (C₁-C₄)alkoxy; benzyloxy; and —OCR^{a5}R^{b5}OC(O)R^{z} (wherein: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached, form a (C₃-C₆)cycloalkyl; and R^{z} is selected from (C₁-C₅)alkyl, (C₁-C₅)alkoxy, and phenyl); and the other R″ is selected from the group defined for R″ according to Formula (II); and
—OP(O)R″R″, wherein one R″ is selected from (C₁-C₄)alkoxy; benzyloxy; and —OCR^{a5}R^{b5}OC(O)R^{z} (wherein: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached, form a (C₃-C₆)cycloalkyl; and R^{z} is selected from (C₁-C₅)alkyl, (C₁-C₅)alkoxy, and phenyl); and the other R″ is selected from the group defined for R″ according to Formula (II).

In some embodiments, R′ is H and R″ is independently selected from —OH, C₁-C₄)alkyl, and (C₁-C₄)alkoxy (in some embodiments, —OH, (C₁-C₄)alkyl (e.g. (C₁-C₂)alkyl), and (C₁-C₄)alkoxy (e.g. (C₁-C₂)alkoxy)).

In some embodiments, R3 is phenyl, pyridyl, pyridazinyl, pyrimidinyl, oxazolyl, tetrazolyl, pyrazolyl, indazolyl, or 1,1-dioxido-2,3-dihydrobenzo[d]isothiazolyl (in more particular embodiments, phenyl, pyridyl, indazolyl, or 1,1-dioxido-2,3-dihydrobenzo[d]isothiazolyl), including the various position isomers thereof, where such groups are optionally substituted in accordance with Formula (II) as defined above, including more particular embodiments of Formula (II).

In some embodiments, R3 is phenyl.

In some embodiments, R3 is phenyl substituted in accordance with the definition of Formula (II), including more particular embodiments of Formula (II). In some embodiments, R3 is 3,4- or 3,5-disubstituted phenyl wherein the substituent groups are selected in accordance with the definition of Formula (II), including more particular embodiments of Formula (II) (said positions relative to the point of attachment of the phenyl ring to the remainder of the compound of Formula (II)).

In some embodiments, R3 is phenyl substituted with one (C₁-C₆)alkoxy group.

In some embodiments, R3 is phenyl substituted with one (C₁-C₆)alkoxy group and one group —C(O)NR^{a2}R^{b2} wherein R^{a2} and R^{b2} are independently selected from H and (C₁-C₄)alkyl, wherein the (C₁-C₄)alkyl is optionally substituted with 1-3 COOH groups. In more particular such embodiments R^{a2} is H and R^{b2} is (C₁-C₄)alkyl, optionally substituted with 1-3 COOH groups (e.g. 2 COOH groups),

In some embodiments, R3 is phenyl substituted with 1-3 (e.g. 1-2) groups independently selected from: —OCH₃, —OC₂H₅, —OC₃H₇, —OCH(CH₃)₂, —OCF₃, —OCHF₂, —OCH₂CF₃, —OCH₂CHF₂, —OC₂H₄— pyrrolidinyl, —OCH₂CO₂H, —OCH₂C(O)NH₂, —CO₂H, —CH₃, cyclopropanyl-1-carboxylic acid, —CH₂CO₂H, —C(CH₃)₂CO₂H, —CH(CH₃)CO₂H, —CF₂CO₂H, —CH₂C(O)NHCH(CO₂H)(CH₂CO₂H), —CH₂P(O)(OH)₂, —CH₂OP(O)(OH)₂, —CH₂N(CH₃)(CH₂CO₂H), —CH₂NHCH₂P(O)(OH)₂, —C(NH₂)(NOH), cyano, nitro, hydroxy, —SO₂NH₂, —SO₂N(CH₃)₂, —SO₂NH(CH₃), —SO₂CH₃, —SO₂NHC(O)C₂H₅, —SCH₃, —SC₂H₅, —C(O)OCH₃, —C(O)OC(CH₃)₃, —C(O)NHCH₃, —C(O)NH(C₂H₄NH₂), —C(O)NHC₂H₄N⁺(CH₃)₃, —C(O)NHCH(CO₂H)(CH₂CO₂H), —C(O)NHCH(CO₂H)(C₂H₄CO₂H), —C(O)NHCH₂CO₂H, —C(O)N(CH₂CO₂H)₂, —C(O)NHCH₂P(O)(OH)₂, —C(O)NHC(CH₂OH)₃, —C(O)pyridyl, fluoro, —NH₂, —N(CH₃)₂, —P(O)(CH₃)(OC₂H₅), —P(O)(OCH₃)₂, —P(O)(CH₃)(OH), —P(O)(OH)(OCH₃), and —P(O)(OH)₂. In more particular embodiments, R3 is phenyl substituted with 1-3 (e.g. 1-2) groups independently selected from —OC₂H₅, —OCH₂CO₂H, —C(O)NHCH(CO₂H)(CH₂CO₂H), and —P(O)(OH)₂.

In some more particular embodiments, R3 is phenyl substituted with 1-2 groups independently selected from —OC₂H₅, —OCH₂CO₂H, —CO₂H, —C(O)NHCH(CO₂H)(CH₂CO₂H), —C(O)NHCH₂P(O)(OH)₂, and —P(O)(OH)₂, in even more particular embodiments independently selected from —OC₂H₅, —OCH₂CO₂H, —C(O)NHCH(CO₂H)(CH₂CO₂H), and —P(O)(OH)₂, in even more particular embodiments independently selected from —OC₂H₅, —OCH₂CO₂H, C(O)NHCH(CO₂H)(CH₂CO₂H), and —P(O)(OH)₂.

In some embodiments, R3 is phenyl substituted with one ethoxy group (in more particular embodiments in the 3-position).

In some embodiments, R3 is phenyl substituted with one ethoxy group and one group —C(O)NHCH(CO₂H)(CH₂CO₂H).

In some embodiments, R3 is phenyl substituted with:

ethoxy in the 3-position, and —P(O)(OH)₂ or —CO₂H in the 4- or 5-position (in more particular embodiments, —P(O)(OH)₂, more particularly in the 5-position);
ethoxy in the 3-position, and —OCH₂CO₂H or —C(O)NHCH(CO₂H)(CH₂CO₂H) in the 4- or 5-position (in more particular embodiments —C(O)NHCH(CO₂H)(CH₂CO₂H), more particularly in the 4-position); or
—OCH₂CO₂H in the 3-position and —C(O)NHCH(CO₂H)(CH₂CO₂H) in the 4 or 5 position (in more particular embodiments, in the 4-position).

In some embodiments, R3 is phenyl substituted with at least one substituent comprising at least one —CO₂R′ group, wherein R′ is (C₁-C₄)alkyl or benzyl.

In some embodiments, R3 is phenyl substituted with at least one substituent comprising at least one group selected from —P(O)R″R″ and —OP(O)R″R″, wherein one or both R″ of said —P(O)R″R″ and —OP(O)R″R″ are independently selected from the group consisting of: (C₁-C₄)alkoxy; benzyloxy; and —OCR^{a5}R^{b5}OC(O)R^{z} (wherein: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl). In more particular embodiments, one or both R″ are independently C₃- or C₄-alkoxy, and/or one or both R″ are independently selected from said benzyloxy and —OCR^{a5}R^{b5}OC(O)R^{z}).

In some embodiments of the compounds of Formula (II), R3 is phenyl either unsubstituted or substituted with 1-2 groups selected from: —COOH, —OEt, —CONHCH(COOH)(CH₂COOH), —PO(OH)₂, —OCH₂COOH and —CONHCH₂PO(OH)₂.

In each of the aforementioned particular embodiments of R3 phenyl and R3 substituted phenyl, in some embodiments R1 is (C₁-C₄) straight chain alkyl (especially ethyl), and R2 is (C₁-C₁₁) alkyl (especially pentyl). As a non-limiting example, in some embodiments R1 is (C₁-C₄) straight chain alkyl (especially ethyl), R2 is (C₁-C₁₁) alkyl (especially pentyl), and R3 is phenyl or phenyl substituted with 1-2 groups independently selected from —OC₂H₅, —OCH₂CO₂H, —CO₂H, —C(O)NHCH(CO₂H)(CH₂CO₂H), —C(O)NHCH₂P(O)(OH)₂, and —P(O)(OH)₂.

In some embodiments, R3 is optionally substituted heteroaryl, e.g.: pyridyl, pyridazinyl, pyrimidinyl, oxazolyl, tetrazolyl, pyrazolyl, indazolyl, or 1,1-dioxido-2,3-dihydrobenzo[d]isothiazolyl. In some embodiments, R3 is optionally substituted pyridyl, indazolyl, or 1,1-dioxido-2,3-dihydrobenzo[d]isothiazolyl (including particularly pyridin-3-yl, pyridin-2-yl, 1,1-dioxido-2,3-dihydrobenzo[d]isothiazol-6-yl, and indazol-6-yl). In these embodiments such R3 groups may be optionally substituted as defined in accordance with Formula II defined above, including more particular embodiments of Formula II.

In some embodiments, heteroaryl R3 is substituted with 1-3 (e.g. 1-2) groups independently selected from: —OCH₃, —OC₂H₅, —OC₃H₇, —OCH(CH₃)₂, —OCF₃, —OCHF₂, —OCH₂CF₃, —OCH₂CHF₂, —OC₂H₄-pyrrolidine, —OCH₂CO₂H, —OCH₂C(O)NH₂, —CO₂H, —CH₃, cyclopropane-1-carboxylic acid, —CH₂CO₂H, —C(CH₃)₂CO₂H, —CH(CH₃)CO₂H, —CF₂CO₂H, —CH₂C(O)NHCH(CO₂H)(CH₂CO₂H), —CH₂P(O)(OH)₂, —CH₂N(CH₃)(CH₂CO₂H), —CH₂NHCH₂P(O)(OH)₂, —C(NH₂)(NOH), cyano, nitro, hydroxy, —SO₂NH₂, —SO₂N(CH₃)₂, —SO₂NH(CH₃), —SO₂CH₃, —SO₂NHC(O)C₂H₅, —SCH₃, —SC₂H₅, —C(O)OCH₃, —C(O)OC(CH₃)₃, —C(O)NHCH₃, —C(O)NH(C₂H₄NH₂), —C(O)NHC₂H₄N⁺(CH₃)₃, —C(O)NHCH(CO₂H)(CH₂CO₂H), —C(O)NHCH(CO₂H)(C₂H₄CO₂H), —C(O)NHCH₂CO₂H, —C(O)N(CH₂CO₂H)₂, —C(O)NHCH₂P(O)(OH)₂, —C(O)NHC(CH₂OH)₃, fluoro, —NH₂, —N(CH₃)₂, —P(O)(CH₃)(OC₂H₅), —P(O)(OCH₃)₂, —P(O)(CH₃)(OH), —P(O)(OH)(OCH₃), and —P(O)(OH)₂. In more particular embodiments, R3 heteroaryl is substituted with 1-3 (e.g. 1-2) —OC₂H₅, —OCH₂CO₂H, —C(O)NHCH(CO₂H)(CH₂CO₂H), —P(O)(OH)₂ or a combination thereof.

In some embodiments, heteroaryl R3 is substituted with:

ethoxy and —P(O)(OH)₂ or —CO₂H (particularly —P(O)(OH)₂); or
ethoxy and —OCH₂CO₂H or —C(O)NHCH(CO₂H)(CH₂CO₂H) (particularly —C(O)NHCH(CO₂H)(CH₂CO₂H); or
—OCH₂CO₂H and —C(O)NHCH(CO₂H)(CH₂CO₂H).

In some embodiments, heteroaryl R3 is substituted with 1-3, e.g., 1-2 groups independently selected from: —OCH₃, —OC₂H₅, —OC₃H₇, —OCH(CH₃)₂, —CO₂H, —CH₃, —P(O)(CH₃)(OC₂H₅), —P(O)(OCH₃)₂, —P(O)(CH₃)(OH), —P(O)(OH)(OCH₃), and —P(O)(OH)₂. In some such embodiments, heteroaryl R3 is substituted with 1-3, e.g., 1-2 groups independently selected from: —OCH₃, —CH₃, and —CO₂H.

In some embodiments, heteroaryl R3 is substituted with at least one substituent comprising at least one —CO₂R′ group, wherein R′ is (C₁-C₄)alkyl or benzyl.

In some embodiments, heteroaryl R3 is substituted with at least one substituent comprising at least one group selected from —P(O)R″R″ and —OP(O)R″R″, wherein one or both R″ of said P(O)R″R″ and —OP(O)R″R″ is independently selected from the group consisting of: (C₁-C₄)alkoxy; benzyloxy; and —OCR^{a5}R^{b5}OC(O)R^{z} (wherein: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached, form a (C₃-C₆)cycloalkyl; and R^{z} is selected from (C₁-C₅)alkyl, (C₁-C₅)alkoxy, and phenyl). In more particular embodiments, one or both R″ is C₃- or C₄-alkoxy, and/or one or both R″ is selected from said benzyloxy and —OCR^{a5}R^{b5}OC(O)R^{z}).

It is to be understood that in relation to particular embodiments of Formula (II), when one R′ or R″ group in a moiety is particularly defined and the other(s) is not, the other(s) may be respectively selected from the group defined for R′ or R″ according to Formula (II).

In some embodiments, all R′ groups in a particular moiety and/or in the compound of Formula (II) are the same. In some embodiments, all R″ groups in a particular moiety and/or in the compound of Formula (II) are the same. In some embodiments, the R′ groups in a particular moiety and/or in the compound of Formula (II) may be different. In some embodiments, the R″ groups in a particular moiety and/or in the compound of Formula (II) may be different. In some embodiments, all the R′ and R″ groups in a particular moiety and/or the compound of Formula (II) are the same. In some embodiments, the R′ and R″ groups in a particular moiety and/or the compound of Formula (II) may be different.

In some embodiments of compounds of Formula (II), R4 is selected from the groups: A. —C(O)R^{x} B. —CR^{a6}R^{b6}OR^{y}; C. (5-methyl-2-oxo-1,3-dioxol-4-yl)methyl; and D. —P(O)R^{z3}R^{z3} as defined above for Formula (II). In more particular embodiments R4 is selected from the groups A. —C(O)R^{x}; B. —CR^{a6}R^{b6}OR^{y}, where R^{y} is —COR^{z1} or —P(O)R^{z2}R^{z2}; C. (5-methyl-2-oxo-1,3-dioxol-4-yl)methyl; and D. —P(O)R^{z3}R^{z3} where R^{z3} is independently selected from phenoxy and NR^{a10}R^{b10} as defined above for Formula (II). In even more particular embodiments, R4 is selected from A. —C(O)R^{x} where R^{x} is optionally substituted phenyl and B. —CR^{a6}R^{b6}OR^{y} where R^{a6} and R^{b6} are H, R^{y} is —P(O)R^{z2}R^{z2}, and each R^{z2} is —OH. In the foregoing embodiments where group A. is optionally substituted phenyl, in more particular embodiments the phenyl substituents are independently selected from (C₁-C₄)alkyl; (C₁-C₄)alkoxy; —(CH₂)₀₋₁heterocycloalkyl containing 1-2 ring nitrogens and/or 1-2 ring oxygens, optionally substituted with one oxo group, and heteroaryl. In some embodiments of the compounds of Formula (II) having the aforementioned R4 groups, R1 is (C₁-C₄) straight chain alkyl (especially ethyl), R2 is (C₁-C₁₁) alkyl (especially pentyl), and R3 is optionally substituted phenyl (especially the particular optionally substituted phenyl embodiments defined herein above and more especially phenyl either unsubstituted or substituted with 1-2 groups selected from: —COOH,—OEt, —CONHCH(COOH)(CH₂COOH), —PO(OH)₂, —OCH₂COOH and —CONHCH₂PO(OH)₂).

In some embodiments of compounds of Formula (II), R4 is selected from: —C(O)phenyl; substituted —C(O)phenyl; —C(O)CH₂phenyl; C(O)t-butyl; —C(O)pyridyl; —COOCH₃; —COOt-butyl; —CH₂OC(O)t-butyl; —CH₂OPO₃H₂; and (5-methyl-2-oxo-1,3-dioxol-4-yl)methyl:

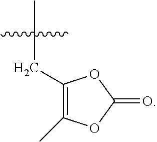 [SMILES: *Cc1oc(=O)oc1C]

In some embodiments of the compounds of Formula (II), R4 is selected from:

—COPh (where Ph is optionally substituted with 1-2 groups selected from: —OH, —COOH, —NO₂, Me, iPr,—CH₂morpholinyl (especially N-linked), —CH₂NH₂, —CF₃, —OMe, —OCF₃, —OC₂H₄OC₂H₄OH, —OCH₂OPO(OH)₂, —OPO(OH)₂, —OC(O)Me, —OC(O)N(Me)₂, —NH₂, —NHMe, —NMeSO₂Me, —N(Me)₂, F, Br, Cl, pyrrolidinyl, optionally substituted with one oxo group, e.g. 2-oxo-pyrrolidinyl, morpholinyl, pyrrolyl, triazolyl, pyrazolyl, and imidazolyl);
—CH₂(2-oxo-1,3-dioxol-4yl);
—CH₂OC(O)C(Me)₃;
—CH₂OPO(OH)₂;

### —COC(Me)₃;

—COC₈H₁₇;

### —COCH(propyl)₂;

### —COCH(Et)₂;

### —COCH₂Ph;

### —COCH(Ph)₂;

—C₀(1-methyl-cyclopropyl);
—CO(2-methyl-cyclopropyl);

### —COnaphthyl;

—COpyridinyl, optionally substituted with 1-2 Me groups;
—CO-isoxazolyl, optionally substituted with 1-2 Me groups;
—COdihydrobenzodioxinyl optionally substituted with 1 Me group;

### —COpiperidinyl;

—CObenzooxazinyl, optionally substituted with one Me group;

### —CONHMe;

### —CONHCH(Me)₂;

### —CONHC(Me)₃;

### —CONHPh;

### —CONHC(Me)₂Ph;

### —CONMeC(O)OC(Me)₃;

### —C(O)OMe;

### —C(O)OC(Me)₃; and

### —PO(OPh)NHCH₂(Me)(COOMe)

(in some more particular embodiments where a substituent includes a heterocyclic ring having a ring N atom, the ring is N-linked to the rest of the molecule).

In some embodiments of the compounds of Formula (II) having any of the aforementioned R4 groups, R1 is (C₁-C₄) straight chain alkyl (especially ethyl), R2 is (C₁-C₁₁) alkyl (especially pentyl), and R3 is optionally substituted phenyl (especially the particular optionally substituted phenyl embodiments defined herein above and more especially phenyl either unsubstituted or substituted with 1-2 groups selected from: —COOH,—OEt, —CONHCH(COOH)(CH₂COOH), —PO(OH)₂, —OCH₂COOH and —CONHCH₂PO(OH)₂).

Accordingly, a compound of the invention may be a compound of Formula (II), or a salt thereof, particularly a pharmaceutically acceptable salt thereof. Representative compounds of the invention include the specific compounds described herein, e.g., the compounds of Formula (II) of the Examples, as well as any alternative stereoisomeric forms, free acid/base forms, salt forms, and alternative salt forms thereof (particularly pharmaceutically acceptable salt or alternative salt forms thereof), as applicable.

Accordingly, in some embodiments the compound of the invention is a compound of Formula (II) selected from the group consisting of:

2-(4-(5-(((2-(1-(N-(benzoyloxy)formamido)propyl)heptanamido)methyl)carbamoyl)furan-2-yl)-
2-ethoxybenzamido)succinic acid
(3-(5-(((2-(1-(N-(benzoyloxy)formamido)propyl)heptanamido)methyl)carbamoyl)furan-2-yl)-5-
ethoxyphenyl)phosphonic acid
(3-ethoxy-5-(5-(((2-(1-(N-(2-
phenylacetoxy)formamido)propyl)heptanamido)methyl)carbamoyl)furan-2-
yl)phenyl)phosphonic acid
2-(2-ethoxy-4-(5-(((2-(1-(N-(2-
phenylacetoxy)formamido)propyl)heptanamido)methyl)carbamoyl)furan-2-
yl)benzamido)succinic acid
2-(2-ethoxy-4-(5-(((2-(1-(N-
(pivaloyloxy)formamido)propyl)heptanamido)methyl)carbamoyl)furan-2-
yl)benzamido)succinic acid
2-(2-(carboxymethoxy)-4-(5-(((2-(1-(N-((2-
hydroxybenzoyl)oxy)formamido)propyl)heptanamido)methyl)carbamoyl)furan-2-
yl)benzamido)succinic acid
2-(4-(5-(((2-(1-(N-(benzoyloxy)formamido)propyl)heptanamido)methyl)carbamoyl)furan-2-yl)-
2-(carboxymethoxy)benzamido)succinic acid
2-(2-(carboxymethoxy)-4-(5-(((2-(1-(N-((2-
methylbenzoyl)oxy)formamido)propyl)heptanamido)methyl)carbamoyl)furan-2-
yl)benzamido)succinic acid
(3-ethoxy-5-(5-((5-ethyl-6-formyl-11,11-dimethyl-3,10-dioxo-4-pentyl-7,9-dioxa-2,6-
diazadodecyl)carbamoyl)furan-2-yl)phenyl)phosphonic acid
(3-ethoxy-5-(5-(((2-(1-(N-((phosphonooxy)methoxy)formamido)propyl)-
heptanamido)methyl)carbamoyl)furan-2-yl)phenyl)phosphonic acid
2-(2-(carboxymethoxy)-4-(5-(((2-(1-(N-
((phosphonooxy)methoxy)formamido)propyl)heptanamido)methyl)carbamoyl)furan-2-
yl)benzamido)succinic acid
2-(4-(5-(((2-(1-(N-((2-
acetoxybenzoyl)oxy)formamido)propyl)heptanamido)methyl)carbamoyl)furan-2-yl)-2-
(carboxymethoxy)benzamido)succinic acid
2-(2-(carboxymethoxy)-4-(5-(((2-(1-(N-
(nicotinoyloxy)formamido)propyl)heptanamido)methyl)carbamoyl)furan-2-
yl)benzamido)succinic acid
2-(2-(carboxymethoxy)-4-(5-((5-ethyl-6-formyl-10,10-dimethyl-3,8-dioxo-4-pentyl-7,9-dioxa-
2,6-diazaundecyl)carbamoyl)furan-2-yl)benzamido)succinic acid
5-phenyl-N-((2-(1-(N-(pivaloyloxy)formamido)propyl)heptanamido)methyl)furan-2-
carboxamide
5-phenyl-N-((2-(1-(N-(2-phenylacetoxy)formamido)propyl)heptanamido)methyl)furan-2-
carboxamide
N-(6-ethyl-5-formyl-3,8-dioxo-7-pentyl-2,4-dioxa-5,9-diazadecan-10-yl)-5-phenylfuran-2-
carboxamide
N-(5-ethyl-6-formyl-10,10-dimethyl-3,8-dioxo-4-pentyl-7,9-dioxa-2,6-diazaundecyl)-5-
phenylfuran-2-carboxamide
N-((2-(1-(N-(benzoyloxy)formamido)propyl)heptanamido)methyl)-5-phenylfuran-2-
carboxamide
(3-ethoxy-5-(5-(((2-(1-(N-((5-methyl-2-oxo-1,3-dioxol-4-
yl)methoxy)formamido)propyl)heptanamido)methyl)carbamoyl)furan-2-yl)phenyl)phosphonic
acid
(3-ethoxy-5-(5-(((2-(1-(N-((2-
hydroxybenzoyl)oxy)formamido)propyl)heptanamido)methyl)carbamoyl)furan-2-
yl)phenyl)phosphonic acid
2-(4-(5-(((2-(1-(N-((3-
carboxybenzoyl)oxy)formamido)propyl)heptanamido)methyl)carbamoyl)furan-2-yl)-2-
(carboxymethoxy)benzamido)succinic acid
2-(2-(carboxymethoxy)-4-(5-(((2-(1-(N-((1-
methylcyclopropanecarbonyl)oxy)formamido)propyl)heptanamido)methyl)carbamoyl)furan-2-
yl)benzamido)succinic acid
2-(2-(carboxymethoxy)-4-(5-(((2-(1-(N-((2-methylcyclopropanecarbonyl)oxy)formamido)propyl)-
heptanamido)methyl)carbamoyl)furan-2-yl)benzamido)succinic acid
2-(2-(carboxymethoxy)-4-(5-(((2-(1-(N-((4-
methoxybenzoyl)oxy)formamido)propyl)heptanamido)methyl)carbamoyl)furan-2-
yl)benzamido)succinic acid
2-(2-(carboxymethoxy)-4-(5-((6-ethyl-5-formyl-3,8-dioxo-7-pentyl-4-oxa-2,5,9-triazadecan-
10-yl)carbamoyl)furan-2-yl)benzamido)succinic acid
2-(4-(5-(((2-(1-(N-((2-
aminobenzoyl)oxy)formamido)propyl)heptanamido)methyl)carbamoyl)furan-2-yl)-2-
(carboxymethoxy)benzamido)succinic acid
2-(2-(carboxymethoxy)-4-(5-(((2-(1-(N-((2-
(methylamino)benzoyl)oxy)formamido)propyl)heptanamido)methyl)carbamoyl)furan-2-
yl)benzamido)succinic acid
2-(2-(carboxymethoxy)-4-(5-(((2-(1-(N-((2-
ethylbutanoyl)oxy)formamido)propyl)heptanamido)methyl)carbamoyl)furan-2-
yl)benzamido)succinic acid
2-(2-(carboxymethoxy)-4-(5-(((2-(1-(N-((3,5-dimethylisoxazole-4-
carbonyl)oxy)formamido)propyl)heptanamido)methyl)carbamoyl)furan-2-
yl)benzamido)succinic acid
2-(2-(carboxymethoxy)-4-(5-(((2-(1-(N-((2,4-
dimethylnicotinoyl)oxy)formamido)propyl)heptanamido)methyl)carbamoyl)furan-2-
yl)benzamido)succinic acid
2-(2-(carboxymethoxy)-4-(5-(((2-(1-(N-(2,2-
diphenylacetoxy)formamido)propyl)heptanamido)methyl)carbamoyl)furan-2-
yl)benzamido)succinic acid
2-(2-(carboxymethoxy)-4-(5-(((2-(1-(N-((2-
isopropylbenzoyl)oxy)formamido)propyl)heptanamido)methyl)carbamoyl)furan-2-
yl)benzamido)succinic acid
2-(2-(carboxymethoxy)-4-(5-((5-ethyl-6-formyl-10,10-dimethyl-3,8-dioxo-4-pentyl-7-oxa-
2,6,9-triazaundecyl)carbamoyl)furan-2-yl)benzamido)succinic acid
2-(2-(carboxymethoxy)-4-(5-((5-ethyl-6-formyl-10-methyl-3,8-dioxo-4-pentyl-7-oxa-2,6,9-
triazaundecyl)carbamoyl)furan-2-yl)benzamido)succinic acid
(3-(5-(((2-(1-(N-((2-
((dimethylcarbamoyl)oxy)benzoyl)oxy)formamido)propyl)heptanamido)methyl)carbamoyl)furan-
2-yl)-5-ethoxyphenyl)phosphonic acid
2-(2-(carboxymethoxy)-4-(5-(((2-(1-(N-((2-
((dimethylcarbamoyl)oxy)benzoyl)oxy)formamido)propyl)heptanamido)methyl)carbamoyl)furan-
2-yl)benzamido)succinic acid
2-(4-(5-(((2-(1-(N-((1-
naphthoyl)oxy)formamido)propyl)heptanamido)methyl)carbamoyl)furan-2-yl)-2-
(carboxymethoxy)benzamido)succinic acid
2-(4-(5-(((2-(1-(N-((2-
naphthoyl)oxy)formamido)propyl)heptanamido)methyl)carbamoyl)furan-2-yl)-2-
(carboxymethoxy)benzamido)succinic acid
2-(2-(carboxymethoxy)-4-(5-(((2-(1-(N-((4-
(morpholinomethyl)benzoyl)oxy)formamido)propyl)heptanamido)methyl)carbamoyl)furan-2-
yl)benzamido)succinic acid
2-(2-(carboxymethoxy)-4-(5-((5-ethyl-6-formyl-10-methyl-3,8-dioxo-4-pentyl-10-phenyl-7-
oxa-2,6,9-triazaundecyl)carbamoyl)furan-2-yl)benzamido)succinic acid
2-(2-(carboxymethoxy)-4-(5-(((2-(1-(N-((((1-methoxy-1-oxopropan-2-
yl)amino)(phenoxy)phosphoryl)oxy)formamido)propyl)heptanamido)methyl)carbamoyl)furan-
2-yl)benzamido)succinic acid
2-(2-(carboxymethoxy)-4-(5-(((2-(1-(N-((2-fluoro-6-
methylbenzoyl)oxy)formamido)propyl)heptanamido)methyl)carbamoyl)furan-2-
yl)benzamido)succinic acid
2-(2-(carboxymethoxy)-4-(5-(((2-(1-(N-((4-methoxy-2-
methylbenzoyl)oxy)formamido)propyl)heptanamido)methyl)carbamoyl)furan-2-
yl)benzamido)succinic acid
2-(2-(carboxymethoxy)-4-(5-(((2-(1-(N-((3-
methylisonicotinoyl)oxy)formamido)propyl)heptanamido)methyl)carbamoyl)furan-2-
yl)benzamido)succinic acid
2-(2-(carboxymethoxy)-4-(5-(((2-(1-(N-((2-methyl-4-
(morpholinomethyl)benzoyl)oxy)formamido)propyl)heptanamido)methyl)carbamoyl)furan-2-
yl)benzamido)succinic acid
2-(2-(carboxymethoxy)-4-(5-(((2-(1-(N-((3,4-
dimethoxybenzoyl)oxy)formamido)propyl)heptanamido)methyl)carbamoyl)furan-2-
yl)benzamido)succinic acid
2-(2-(carboxymethoxy)-4-(5-(((2-(1-(N-((2-
propylpentanoyl)oxy)formamido)propyl)heptanamido)methyl)carbamoyl)furan-2-
yl)benzamido)succinic acid
2-(2-(carboxymethoxy)-4-(5-(((2-(1-(N-((2,4-
dimethoxybenzoyl)oxy)formamido)propyl)heptanamido)methyl)carbamoyl)furan-2-
yl)benzamido)succinic acid
(3-ethoxy-5-(5-(((2-(1-(N-((2-
ethylbutanoyl)oxy)formamido)propyl)heptanamido)methyl)carbamoyl)furan-2-
yl)phenyl)phosphonic acid
(3-ethoxy-5-(5-(((2-(1-(N-((2-fluoro-6-
methylbenzoyl)oxy)formamido)propyl)heptanamido)methyl)carbamoyl)furan-2-
yl)phenyl)phosphonic acid
(3-ethoxy-5-(5-(((2-(1-(N-((4-
methoxybenzoyl)oxy)formamido)propyl)heptanamido)methyl)carbamoyl)furan-2-
yl)phenyl)phosphonic acid
2-(2-ethoxy-4-(5-(((2-(1-(N-((2-
ethylbutanoyl)oxy)formamido)propyl)heptanamido)methyl)carbamoyl)furan-2-
yl)benzamido)succinic acid
2-(2-ethoxy-4-(5-(((2-(1-(N-((2-fluoro-6-
methylbenzoyl)oxy)formamido)propyl)heptanamido)methyl)carbamoyl)furan-2-
yl)benzamido)succinic acid
(3-ethoxy-5-(5-(((2-(1-(N-((4-(pyrrolidin-1-
yl)benzoyl)oxy)formamido)propyl)heptanamido)methyl)carbamoyl)furan-2-
yl)phenyl)phosphonic acid
2-(2-ethoxy-4-(5-(((2-(1-(N-((4-
methoxybenzoyl)oxy)formamido)propyl)heptanamido)methyl)carbamoyl)furan-2-
yl)benzamido)succinic acid
2-(2-(carboxymethoxy)-4-(5-(((2-(1-(N-((2-isopropyl-4-
methoxybenzoyl)oxy)formamido)propyl)heptanamido)methyl)carbamoyl)furan-2-
yl)benzamido)succinic acid
2-(2-(carboxymethoxy)-4-(5-(((2-(1-(N-((4-fluoro-2-
isopropylbenzoyl)oxy)formamido)propyl)heptanamido)methyl)carbamoyl)furan-2-
yl)benzamido)succinic acid
2-(2-ethoxy-4-(5-(((2-(1-(N-((4-methoxy-2-
methylbenzoyl)oxy)formamido)propyl)heptanamido)methyl)carbamoyl)furan-2-
yl)benzamido)succinic acid
(3-ethoxy-5-(5-((5-ethyl-6-formyl-10,10-dimethyl-3,8-dioxo-4-pentyl-7-oxa-2,6,9-
triazaundecyl)carbamoyl)furan-2-yl)phenyl)phosphonic acid
(3-ethoxy-5-(5-((5-ethyl-6-formyl-10-methyl-3,8-dioxo-4-pentyl-10-phenyl-7-oxa-2,6,9-
triazaundecyl)carbamoyl)furan-2-yl)phenyl)phosphonic acid
(3-ethoxy-5-(5-(((2-(1-(N-((4-
morpholinobenzoyl)oxy)formamido)propyl)heptanamido)methyl)carbamoyl)furan-2-
yl)phenyl)phosphonic acid
(3-(5-(((2-(1-(N-((4-(1H-pyrazol-1-
yl)benzoyl)oxy)formamido)propyl)heptanamido)methyl)carbamoyl)furan-2-yl)-5-
ethoxyphenyl)phosphonic acid
(3-ethoxy-5-(5-(((2-(1-(N-((4-(2-oxopyrrolidin-1-
yl)benzoyl)oxy)formamido)propyl)heptanamido)methyl)carbamoyl)furan-2-
yl)phenyl)phosphonic acid
(3-ethoxy-5-(5-(((2-(1-(N-((4-methyl-3,4-dihydro-2H-benzo[b][1,4]oxazine-7-
carbonyl)oxy)formamido)propyl)heptanamido)methyl)carbamoyl)furan-2-
yl)phenyl)phosphonic acid
(3-ethoxy-5-(5-(((2-(1-(N-((2-methyl-6-
((phosphonooxy)methoxy)benzoyl)oxy)formamido)propyl)heptanamido)methyl)carbamoyl)furan-
2-yl)phenyl)phosphonic acid
(3-ethoxy-5-(5-(((2-(1-(N-((3-fluoro-4-(pyrrolidin-1-
yl)benzoyl)oxy)formamido)propyl)heptanamido)methyl)carbamoyl)furan-2-
yl)phenyl)phosphonic acid
(3-ethoxy-5-(5-(((2-(1-(N-((3-(pyrrolidin-1-
yl)benzoyl)oxy)formamido)propyl)heptanamido)methyl)carbamoyl)furan-2-
yl)phenyl)phosphonic acid
(3-ethoxy-5-(5-(((2-(1-(N-((2-methyl-4-
(morpholinomethyl)benzoyl)oxy)formamido)propyl)heptanamido)methyl)carbamoyl)furan-2-
yl)phenyl)phosphonic acid
(3-ethoxy-5-(5-((5-ethyl-6-formyl-10-methyl-3,8-dioxo-4-pentyl-7-oxa-2,6,9-
triazaundecyl)carbamoyl)furan-2-yl)phenyl)phosphonic acid
2-(2-ethoxy-4-(5-(((2-(1-(N-((2-isopropyl-4-
methoxybenzoyl)oxy)formamido)propyl)heptanamido)methyl)carbamoyl)furan-2-
yl)benzamido)succinic acid
2-(2-ethoxy-4-(5-(((2-(1-(N-((4-fluoro-2-
isopropylbenzoyl)oxy)formamido)propyl)heptanamido)methyl)carbamoyl)furan-2-
yl)benzamido)succinic acid
(3-ethoxy-5-(5-(((2-(1-(N-((4-fluoro-2-
isopropylbenzoyl)oxy)formamido)propyl)heptanamido)methyl)carbamoyl)furan-2-
yl)phenyl)phosphonic acid
(3-ethoxy-5-(5-(((2-(1-(N-
(nonanoyloxy)formamido)propyl)heptanamido)methyl)carbamoyl)furan-2-
yl)phenyl)phosphonic acid
2-(2-ethoxy-4-(5-(((2-(1-(N-((4-
(morpholinomethyl)benzoyl)oxy)formamido)propyl)heptanamido)methyl)carbamoyl)furan-2-
yl)benzamido)succinic acid
2-(2-ethoxy-4-(5-(((2-(1-(N-((2-methyl-4-
(morpholinomethyl)benzoyl)oxy)formamido)propyl)heptanamido)methyl)carbamoyl)furan-2-
yl)benzamido)succinic acid
2-(2-ethoxy-4-(5-((5-ethyl-6-formyl-10-methyl-3,8-dioxo-4-pentyl-7-oxa-2,6,9-
triazaundecyl)carbamoyl)furan-2-yl)benzamido)succinic acid
(3-ethoxy-5-(5-(((2-(1-(N-((4-
(morpholinomethyl)benzoyl)oxy)formamido)propyl)heptanamido)methyl)carbamoyl)furan-2-
yl)phenyl)phosphonic acid
2-(2-ethoxy-4-(5-((5-ethyl-6-formyl-10,10-dimethyl-3,8-dioxo-4-pentyl-7-oxa-2,6,9-
triazaundecyl)carbamoyl)furan-2-yl)benzamido)succinic acid
2-(2-ethoxy-4-(5-((5-ethyl-6-formyl-10-methyl-3,8-dioxo-4-pentyl-10-phenyl-7-oxa-2,6,9-
triazaundecyl)carbamoyl)furan-2-yl)benzamido)succinic acid
2-(2-ethoxy-4-(5-(((2-(1-(N-((3-(pyrrolidin-1-
yl)benzoyl)oxy)formamido)propyl)heptanamido)methyl)carbamoyl)furan-2-
yl)benzamido)succinic acid
2-(2-ethoxy-4-(5-(((2-(1-(N-((3-fluoro-4-(pyrrolidin-1-
yl)benzoyl)oxy)formamido)propyl)heptanamido)methyl)carbamoyl)furan-2-
yl)benzamido)succinic acid
2-(2-ethoxy-4-(5-(((2-(1-(N-((4-(2-oxopyrrolidin-1-
yl)benzoyl)oxy)formamido)propyl)heptanamido)methyl)carbamoyl)furan-2-
yl)benzamido)succinic acid
2-(4-(5-(((2-(1-(N-((4-(1H-pyrazol-1-
yl)benzoyl)oxy)formamido)propyl)heptanamido)methyl)carbamoyl)furan-2-yl)-2-
ethoxybenzamido)succinic acid
2-(2-ethoxy-4-(5-(((2-(1-(N-
((phosphonooxy)methoxy)formamido)propyl)heptanamido)methyl)carbamoyl)furan-2-
yl)benzamido)succinic acid
(3-(5-(((2-(1-(N-((2,3-dihydrobenzo[b][1,4]dioxine-6-
carbonyl)oxy)formamido)propyl)heptanamido)methyl)carbamoyl)furan-2-yl)-5-
ethoxyphenyl)phosphonic acid
(3-ethoxy-5-(5-((5-ethyl-6-formyl-10,10-dimethyl-3,8-dioxo-4-pentyl-7,9-dioxa-2,6-
diazaundecyl)carbamoyl)furan-2-yl)phenyl)phosphonic acid
2-(4-(5-(((2-(1-(N-((2,3-dihydrobenzo[b][1,4]dioxine-6-
carbonyl)oxy)formamido)propyl)heptanamido)methyl)carbamoyl)furan-2-yl)-2-
ethoxybenzamido)succinic acid
(3-ethoxy-5-(5-(((2-(1-(N-((4-methyl-3,4-dihydro-2H-benzo[b][1,4]oxazine-6-
carbonyl)oxy)formamido)propyl)heptanamido)methyl)carbamoyl)furan-2-
yl)phenyl)phosphonic acid
2-(2-ethoxy-4-(5-(((2-(1-(N-((4-methyl-3,4-dihydro-2H-benzo[b][1,4]oxazine-6-
carbonyl)oxy)formamido)propyl)heptanamido)methyl)carbamoyl)furan-2-
yl)benzamido)succinic acid
2-(2-ethoxy-4-(5-((5-ethyl-6-formyl-10,10-dimethyl-3,8-dioxo-4-pentyl-7,9-dioxa-2,6-
diazaundecyl)carbamoyl)furan-2-yl)benzamido)succinic acid
2-(2-(carboxymethoxy)-4-(5-(((2-(1-(N-((2-methoxy-6-
methylbenzoyl)oxy)formamido)propyl)heptanamido)methyl)carbamoyl)tetrahydrofuran-2-
yl)benzamido)succinic acid
(3-ethoxy-5-(5-(((2-(1-(N-((4-methoxy-2-
methylbenzoyl)oxy)formamido)propyl)heptanamido)methyl)carbamoyl)furan-2-
yl)phenyl)phosphonic acid
(3-ethoxy-5-(5-(((2-(1-(N-((2-isopropyl-4-
methoxybenzoyl)oxy)formamido)propyl)heptanamido)methyl)carbamoyl)furan-2-
yl)phenyl)phosphonic acid
2-(2-(carboxymethoxy)-4-(5-(((2-(1-(N-
((phenylcarbamoyl)oxy)formamido)propyl)heptanamido)methyl)carbamoyl)furan-2-
yl)benzamido)succinic acid
2-(4-(5-(((2-(1-(N-((3,4-
dimethoxybenzoyl)oxy)formamido)propyl)heptanamido)methyl)carbamoyl)furan-2-yl)-2-
ethoxybenzamido)succinic acid
(3-ethoxy-5-(5-(((2-(1-(N-
((phenylcarbamoyl)oxy)formamido)propyl)heptanamido)methyl)carbamoyl)furan-2-
yl)phenyl)phosphonic acid
2-(2-ethoxy-4-(5-(((2-(1-(N-((2-
methylbenzoyl)oxy)formamido)propyl)heptanamido)methyl)carbamoyl)furan-2-
yl)benzamido)succinic acid
2-(2-ethoxy-4-(5-((5-ethyl-6-formyl-11,11-dimethyl-3,10-dioxo-4-pentyl-7,9-dioxa-2,6-
diazadodecyl)carbamoyl)furan-2-yl)benzamido)succinic acid
N-{[2-(carboxymethoxy)-4-(5-{[5-ethyl-6-formyl-9,12,12-trimethyl-3,8,10-trioxo-4-pentyl-7,11-
dioxa-2,6,9-triazatridec-1-yl]carbamoyl}furan-2-yl)phenyl]carbonyl}-L-aspartic acid
2-(4-(5-(((2-(1-(N-((2,4-
dimethoxybenzoyl)oxy)formamido)propyl)heptanamido)methyl)carbamoyl)furan-2-yl)-2-
ethoxybenzamido)succinic acid
(3-ethoxy-5-(5-(((2-(1-(N-((2-methyl-4-
morpholinobenzoyl)oxy)formamido)propyl)heptanamido)methyl)carbamoyl)furan-2-
yl)phenyl)phosphonic acid
2-(2-ethoxy-4-(5-(((2-(1-(N-((2-
isopropylbenzoyl)oxy)formamido)propyl)heptanamido)methyl)carbamoyl)furan-2-
yl)benzamido)succinic acid
2-(4-(5-(((2-(1-(N-((2,4-
dimethylnicotinoyl)oxy)formamido)propyl)heptanamido)methyl)carbamoyl)furan-2-yl)-2-
ethoxybenzamido)succinic acid
2-(2-ethoxy-4-(5-(((2-(1-(N-((4-fluoro-2-
methylbenzoyl)oxy)formamido)propyl)heptanamido)methyl)carbamoyl)furan-2-
yl)benzamido)succinic acid
2-(2-ethoxy-4-(5-(((2-(1-(N-((piperidine-4-
carbonyl)oxy)formamido)propyl)heptanamido)methyl)carbamoyl)furan-2-
yl)benzamido)succinic acid
2-(2-ethoxy-4-(5-(((2-(1-(N-((4-
(trifluoromethyl)benzoyl)oxy)formamido)propyl)heptanamido)methyl)carbamoyl)furan-2-
yl)benzamido)succinic acid
2-(2-ethoxy-4-(5-(((2-(1-(N-((4-
nitrobenzoyl)oxy)formamido)propyl)heptanamido)methyl)carbamoyl)furan-2-
yl)benzamido)succinic acid
2-(2-ethoxy-4-(5-(((2-(1-(N-((4-
hydroxybenzoyl)oxy)formamido)propyl)heptanamido)methyl)carbamoyl)furan-2-
yl)benzamido)succinic acid
2-(2-ethoxy-4-(5-(((2-(1-(N-((2-fluoro-4-
methoxybenzoyl)oxy)formamido)propyl)heptanamido)methyl)carbamoyl)furan-2-
yl)benzamido)succinic acid
2-(2-ethoxy-4-(5-(((2-(1-(N-((4-methoxy-2-
(trifluoromethyl)benzoyl)oxy)formamido)propyl)heptanamido)methyl)carbamoyl)furan-2-
yl)benzamido)succinic acid
2-(2-ethoxy-4-(5-(((2-(1-(N-((4-methoxy-2-
(trifluoromethoxy)benzoyl)oxy)formamido)propyl)heptanamido)methyl)carbamoyl)furan-2-
yl)benzamido)succinic acid
((4-(5-(((2-(1-(N-(benzoyloxy)formamido)propyl)heptanamido)methyl)carbamoyl)furan-2-yl)-
2-ethoxybenzamido)methyl)phosphonic acid
2-(2-ethoxy-4-(5-(((2-(1-(N-((2-methyl-4-
(trifluoromethyl)benzoyl)oxy)formamido)propyl)heptanamido)methyl)carbamoyl)furan-2-
yl)benzamido)succinic acid
2-(4-(5-(((2-(1-(N-((4-(4H-1,2,4-triazol-4-
yl)benzoyl)oxy)formamido)propyl)heptanamido)methyl)carbamoyl)furan-2-yl)-2-
ethoxybenzamido)succinic acid
2-(4-(5-(((2-(1-(N-((3-(1H-pyrrol-1-
yl)benzoyl)oxy)formamido)propyl)heptanamido)methyl)carbamoyl)furan-2-yl)-2-
ethoxybenzamido)succinic acid
2-(2-ethoxy-4-(5-(((2-(1-(N-((4-(N-
methylmethylsulfonamido)benzoyl)oxy)formamido)propyl)heptanamido)methyl)carbamoyl)furan-
2-yl)benzamido)succinic acid
2-(4-(5-(((2-(1-(N-((4-(1H-pyrazol-1-
yl)benzoyl)oxy)formamido)propyl)heptanamido)methyl)carbamoyl)furan-2-yl)-2-
ethoxybenzamido)succinic acid
2-(4-(5-(((2-(1-(N-((4-(1H-imidazol-1-
yl)benzoyl)oxy)formamido)propyl)heptanamido)methyl)carbamoyl)furan-2-yl)-2-
ethoxybenzamido)succinic acid
((2-ethoxy-4-(5-(((2-(1-(N-((4-
methoxybenzoyl)oxy)formamido)propyl)heptanamido)methyl)carbamoyl)furan-2-
yl)benzamido)methyl)phosphonic acid
((2-ethoxy-4-(5-(((2-(1-(N-((4-methoxy-2-
methylbenzoyl)oxy)formamido)propyl)heptanamido)methyl)carbamoyl)furan-2-
yl)benzamido)methyl)phosphonic acid
((2-ethoxy-4-(5-(((2-(1-(N-((2-methyl-4-
(morpholinomethyl)benzoyl)oxy)formamido)propyl)heptanamido)methyl)carbamoyl)furan-2-
yl)benzamido)methyl)phosphonic acid
2-(2-ethoxy-4-(5-(((2-(1-(N-((4-(2-(2-
hydroxyethoxy)ethoxy)benzoyl)oxy)formamido)propyl)heptanamido)methyl)carbamoyl)furan-
2-yl)benzamido)succinic acid
4-ethyl-3-formyl-6,10-dioxo-5-pentyl-10-(5-phenylfuran-2-yl)-2-oxa-3,7,9-triazadecyl
dihydrogen phosphate
2-(2-ethoxy-4-(5-(((2-(1-(N-((2-methyl-6-
(phosphonooxy)benzoyl)oxy)formamido)propyl)heptanamido)methyl)carbamoyl)furan-2-
yl)benzamido)succinic acid
4-(5-(((2-(1-(N-(benzoyloxy)formamido)propyl)heptanamido)methyl)carbamoyl)furan-2-
yl)benzoic acid
2-(2-ethoxy-4-(5-(((2-(1-(N-
((phenylcarbamoyl)oxy)formamido)propyl)heptanamido)methyl)carbamoyl)furan-2-
yl)benzamido)succinic acid
2-(4-(5-(((2-(1-(N-((2-bromo-4-
methoxybenzoyl)oxy)formamido)propyl)heptanamido)methyl)carbamoyl)furan-2-yl)-2-
ethoxybenzamido)succinic acid
2-(4-(5-(((2-(1-(N-((2-chloro-4-
methoxybenzoyl)oxy)formamido)propyl)heptanamido)methyl)carbamoyl)furan-2-yl)-2-
ethoxybenzamido)succinic acid
2-(2-ethoxy-4-(5-(((2-(1-(N-((4-
(methylamino)benzoyl)oxy)formamido)propyl)heptanamido)methyl)carbamoyl)furan-2-
yl)benzamido)succinic acid
2-(4-(5-(((2-(1-(N-((4-
aminobenzoyl)oxy)formamido)propyl)heptanamido)methyl)carbamoyl)furan-2-yl)-2-
ethoxybenzamido)succinic acid
2-(4-(5-(((2-(1-(N-((4-
(aminomethyl)benzoyl)oxy)formamido)propyl)heptanamido)methyl)carbamoyl)furan-2-yl)-2-
ethoxybenzamido)succinic acid
2-(2-ethoxy-4-(5-(((2-(1-(N-((2-methyl-4-
nitrobenzoyl)oxy)formamido)propyl)heptanamido)methyl)carbamoyl)furan-2-
yl)benzamido)succinic acid
2-(4-(5-(((2-(1-(N-((3-
aminobenzoyl)oxy)formamido)propyl)heptanamido)methyl)carbamoyl)furan-2-yl)-2-
ethoxybenzamido)succinic acid
2-(4-(5-(((2-(1-(N-((3-
(dimethylamino)benzoyl)oxy)formamido)propyl)heptanamido)methyl)carbamoyl)furan-2-yl)-
2-ethoxybenzamido)succinic acid; and
2-(2-ethoxy-4-(5-(((2-(1-(N-((3-
(methylamino)benzoyl)oxy)formamido)propyl)heptanamido)methyl)carbamoyl)furan-2-
yl)benzamido)succinic acid

or a salt (e.g., a pharmaceutically acceptable salt) thereof.

In some embodiments the compound of the invention is a compound of Formula (II) selected from the group consisting of:

(S)-2-(4-(5-((((R)-2-((R)-1-(N-
(benzoyloxy)formamido)propyl)heptanamido)methyl)carbamoyl)furan-2-yl)-2-
ethoxybenzamido)succinic acid
(3-(5-((((R)-2-((R)-1-(N-
(benzoyloxy)formamido)propyl)heptanamido)methyl)carbamoyl)furan-2-yl)-5-
ethoxyphenyl)phosphonic acid
(3-ethoxy-5-(5-((((R)-2-((R)-1-(N-(2-
phenylacetoxy)formamido)propyl)heptanamido)methyl)carbamoyl)furan-2-
yl)phenyl)phosphonic acid
(S)-2-(2-ethoxy-4-(5-((((R)-2-((R)-1-(N-(2-
phenylacetoxy)formamido)propyl)heptanamido)methyl)carbamoyl)furan-2-
yl)benzamido)succinic acid
(S)-2-(2-ethoxy-4-(5-((((R)-2-((R)-1-(N-
(pivaloyloxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-
hydroxybenzoyl)oxy)formamido)propyl)heptanamido)methyl)carbamoyl)furan-2-
yl)benzamido)succinic acid
(S)-2-(4-(5-((((R)-2-((R)-1-(N-
(benzoyloxy)formamido)propyl)heptanamido)methyl)carbamoyl)furan-2-yl)-2-
(carboxymethoxy)benzamido)succinic acid
(S)-2-(2-(carboxymethoxy)-4-(5-((((R)-2-((R)-1-(N-((2-
methylbenzoyl)oxy)formamido)propyl)heptanamido)methyl)carbamoyl)furan-2-
yl)benzamido)succinic acid
(3-ethoxy-5-(5-(((4R,5R)-5-ethyl-6-formyl-11,11-dimethyl-3,10-dioxo-4-pentyl-7,9-dioxa-2,6-
diazadodecyl)carbamoyl)furan-2-yl)phenyl)phosphonic acid
(3-ethoxy-5-(5-((((R)-2-((R)-1-(N-
((phosphonooxy)methoxy)formamido)propyl)heptanamido)methyl)carbamoyl)furan-
2-yl)phenyl)phosphonic acid
(S)-2-(2-(carboxymethoxy)-4-(5-((((R)-2-((R)-1-(N-
((phosphonooxy)methoxy)formamido)propyl)heptanamido)methyl)carbamoyl)furan-2-
yl)benzamido)succinic acid
(S)-2-(4-(5-((((R)-2-((R)-1-(N-((2-
acetoxybenzoyl)oxy)formamido)propyl)heptanamido)methyl)carbamoyl)furan-2-yl)-2-
(carboxymethoxy)benzamido)succinic acid
(S)-2-(2-(carboxymethoxy)-4-(5-((((R)-2-((R)-1-(N-
(nicotinoyloxy)formamido)propyl)heptanamido)methyl)carbamoyl)furan-2-
yl)benzamido)succinic acid
(S)-2-(2-(carboxymethoxy)-4-(5-(((4R,5R)-5-ethyl-6-formyl-10,10-dimethyl-3,8-dioxo-4-
pentyl-7,9-dioxa-2,6-diazaundecyl)carbamoyl)furan-2-yl)benzamido)succinic acid
5-phenyl-N-(((R)-2-((R)-1-(N-(pivaloyloxy)formamido)propyl)heptanamido)methyl)furan-2-
carboxamide
5-phenyl-N-(((R)-2-((R)-1-(N-(2-
phenylacetoxy)formamido)propyl)heptanamido)methyl)furan-2-carboxamide
N-((6R,7R)-6-ethyl-5-formyl-3,8-dioxo-7-pentyl-2,4-dioxa-5,9-diazadecan-10-yl)-5-
phenylfuran-2-carboxamide
N-((4R,5R)-5-ethyl-6-formyl-10,10-dimethyl-3,8-dioxo-4-pentyl-7,9-dioxa-2,6-diazaundecyl)-
5-phenylfuran-2-carboxamide
N-(((R)-2-((R)-1-(N-(benzoyloxy)formamido)propyl)heptanamido)methyl)-5-phenylfuran-2-
carboxamide
(3-ethoxy-5-(5-((((R)-2-((R)-1-(N-((5-methyl-2-oxo-1,3-dioxol-4-
yl)methoxy)formamido)propyl)heptanamido)methyl)carbamoyl)furan-2-yl)phenyl)phosphonic
acid
(3-ethoxy-5-(5-((((R)-2-((R)-1-(N-((2-
hydroxybenzoyl)oxy)formamido)propyl)heptanamido)methyl)carbamoyl)furan-2-
yl)phenyl)phosphonic acid
(S)-2-(4-(5-((((R)-2-((R)-1-(N-((3-
carboxybenzoyl)oxy)formamido)propyl)heptanamido)methyl)carbamoyl)furan-2-yl)-2-
(carboxymethoxy)benzamido)succinic acid
(S)-2-(2-(carboxymethoxy)-4-(5-((((R)-2-((R)-1-(N-((1-
methylcyclopropanecarbonyl)oxy)formamido)propyl)heptanamido)methyl)carbamoyl)furan-2-
yl)benzamido)succinic acid
(2S)-2-(2-(carboxymethoxy)-4-(5-((((2R)-2-((1R)-1-(N-
((2-methylcyclopropanecarbonyl)oxy)formamido)propyl)heptanamido)methyl)carbamoyl)furan-
2-yl)benzamido)succinic acid
(S)-2-(2-(carboxymethoxy)-4-(5-((((R)-2-((R)-1-(N-((4-
methoxybenzoyl)oxy)formamido)propyl)heptanamido)methyl)carbamoyl)furan-2-
yl)benzamido)succinic acid
(S)-2-(2-(carboxymethoxy)-4-(5-(((6R,7R)-6-ethyl-5-formyl-3,8-dioxo-7-pentyl-4-oxa-2,5,9-
triazadecan-10-yl)carbamoyl)furan-2-yl)benzamido)succinic acid
(S)-2-(4-(5-((((R)-2-((R)-1-(N-((2-
aminobenzoyl)oxy)formamido)propyl)heptanamido)methyl)carbamoyl)furan-2-yl)-2-
(carboxymethoxy)benzamido)succinic acid
(S)-2-(2-(carboxymethoxy)-4-(5-((((R)-2-((R)-1-(N-((2-
(methylamino)benzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-
ethylbutanoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((3,5-dimethylisoxazole-4-
carbonyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2,4-
dimethylnicotinoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-(2,2-
diphenylacetoxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-
isopropylbenzoyl)oxy)formamido)propyl)heptanamido)methyl)carbamoyl)furan-2-
yl)benzamido)succinic acid
(S)-2-(2-(carboxymethoxy)-4-(5-(((4R,5R)-5-ethyl-6-formyl-10,10-dimethyl-3,8-dioxo-4-
pentyl-7-oxa-2,6,9-triazaundecyl)carbamoyl)furan-2-yl)benzamido)succinic acid
(S)-2-(2-(carboxymethoxy)-4-(5-(((4R,5R)-5-ethyl-6-formyl-10-methyl-3,8-dioxo-4-pentyl-7-
oxa-2,6,9-triazaundecyl)carbamoyl)furan-2-yl)benzamido)succinic acid
(3-(5-((((R)-2-((R)-1-(N-((2-
((dimethylcarbamoyl)oxy)benzoyl)oxy)formamido)propyl)heptanamido)methyl)carbamoyl)furan-
2-yl)-5-ethoxyphenyl)phosphonic acid
(S)-2-(2-(carboxymethoxy)-4-(5-((((R)-2-((R)-1-(N-((2-
((dimethylcarbamoyl)oxy)benzoyl)oxy)formamido)propyl)heptanamido)methyl)carbamoyl)furan-
2-yl)benzamido)succinic acid
(S)-2-(4-(5-((((R)-2-((R)-1-(N-((1-
naphthoyl)oxy)formamido)propyl)heptanamido)methyl)carbamoyl)furan-2-yl)-2-
(carboxymethoxy)benzamido)succinic acid
(S)-2-(4-(5-((((R)-2-((R)-1-(N-((2-
naphthoyl)oxy)formamido)propyl)heptanamido)methyl)carbamoyl)furan-2-yl)-2-
(carboxymethoxy)benzamido)succinic acid
(S)-2-(2-(carboxymethoxy)-4-(5-((((R)-2-((R)-1-(N-((4-
(morpholinomethyl)benzoyl)oxy)formamido)propyl)heptanamido)methyl)carbamoyl)furan-2-
yl)benzamido)succinic acid
(S)-2-(2-(carboxymethoxy)-4-(5-(((4R,5R)-5-ethyl-6-formyl-10-methyl-3,8-dioxo-4-pentyl-10-
phenyl-7-oxa-2,6,9-triazaundecyl)carbamoyl)furan-2-yl)benzamido)succinic acid
(2S)-2-(2-(carboxymethoxy)-4-(5-((((2R)-2-((1R)-1-(N-(((((S)-1-methoxy-1-oxopropan-2-
yl)amino)(phenoxy)phosphoryl)oxy)formamido)propyl)heptanamido)methyl)carbamoyl)furan-
2-yl)benzamido)succinic acid
(S)-2-(2-(carboxymethoxy)-4-(5-((((R)-2-((R)-1-(N-((2-fluoro-6-
methylbenzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((4-methoxy-2-
methylbenzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((3-
methylisonicotinoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-methyl-4-
(morpholinomethyl)benzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((3,4-
dimethoxybenzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-
propylpentanoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2,4-
dimethoxybenzoyl)oxy)formamido)propyl)heptanamido)methyl)carbamoyl)furan-2-
yl)benzamido)succinic acid
(3-ethoxy-5-(5-((((R)-2-((R)-1-(N-((2-
ethylbutanoyl)oxy)formamido)propyl)heptanamido)methyl)carbamoyl)furan-2-
yl)phenyl)phosphonic acid
(3-ethoxy-5-(5-((((R)-2-((R)-1-(N-((2-fluoro-6-
methylbenzoyl)oxy)formamido)propyl)heptanamido)methyl)carbamoyl)furan-2-
yl)phenyl)phosphonic acid
(3-ethoxy-5-(5-((((R)-2-((R)-1-(N-((4-
methoxybenzoyl)oxy)formamido)propyl)heptanamido)methyl)carbamoyl)furan-2-
yl)phenyl)phosphonic acid
(S)-2-(2-ethoxy-4-(5-((((R)-2-((R)-1-(N-((2-
ethylbutanoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((2-fluoro-6-
methylbenzoyl)oxy)formamido)propyl)heptanamido)methyl)carbamoyl)furan-2-
yl)benzamido)succinic acid
(3-ethoxy-5-(5-((((R)-2-((R)-1-(N-((4-(pyrrolidin-1-
yl)benzoyl)oxy)formamido)propyl)heptanamido)methyl)carbamoyl)furan-2-
yl)phenyl)phosphonic acid
(S)-2-(2-ethoxy-4-(5-((((R)-2-((R)-1-(N-((4-
methoxybenzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-isopropyl-4-
methoxybenzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((4-fluoro-2-
isopropylbenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-methoxy-2-
methylbenzoyl)oxy)formamido)propyl)heptanamido)methyl)carbamoyl)furan-2-
yl)benzamido)succinic acid
(3-ethoxy-5-(5-(((4R,5R)-5-ethyl-6-formyl-10,10-dimethyl-3,8-dioxo-4-pentyl-7-oxa-2,6,9-
triazaundecyl)carbamoyl)furan-2-yl)phenyl)phosphonic acid
(3-ethoxy-5-(5-(((4R,5R)-5-ethyl-6-formyl-10-methyl-3,8-dioxo-4-pentyl-10-phenyl-7-oxa-
2,6,9-triazaundecyl)carbamoyl)furan-2-yl)phenyl)phosphonic acid
(3-ethoxy-5-(5-((((R)-2-((R)-1-(N-((4-
morpholinobenzoyl)oxy)formamido)propyl)heptanamido)methyl)carbamoyl)furan-2-
yl)phenyl)phosphonic acid
(3-(5-((((R)-2-((R)-1-(N-((4-(1H-pyrazol-1-
yl)benzoyl)oxy)formamido)propyl)heptanamido)methyl)carbamoyl)furan-2-yl)-5-
ethoxyphenyl)phosphonic acid
(3-ethoxy-5-(5-((((R)-2-((R)-1-(N-((4-(2-oxopyrrolidin-1-
yl)benzoyl)oxy)formamido)propyl)heptanamido)methyl)carbamoyl)furan-2-
yl)phenyl)phosphonic acid
(3-ethoxy-5-(5-((((R)-2-((R)-1-(N-((4-methyl-3,4-dihydro-2H-benzo[b][1,4]oxazine-7-
carbonyl)oxy)formamido)propyl)heptanamido)methyl)carbamoyl)furan-2-
yl)phenyl)phosphonic acid
(3-ethoxy-5-(5-((((R)-2-((R)-1-(N-((2-methyl-6-
((phosphonooxy)methoxy)benzoyl)oxy)formamido)propyl)heptanamido)methyl)carbamoyl)furan-
2-yl)phenyl)phosphonic acid
(3-ethoxy-5-(5-((((R)-2-((R)-1-(N-((3-fluoro-4-(pyrrolidin-1-
yl)benzoyl)oxy)formamido)propyl)heptanamido)methyl)carbamoyl)furan-2-
yl)phenyl)phosphonic acid
(3-ethoxy-5-(5-((((R)-2-((R)-1-(N-((3-(pyrrolidin-1-
yl)benzoyl)oxy)formamido)propyl)heptanamido)methyl)carbamoyl)furan-2-
yl)phenyl)phosphonic acid
(3-ethoxy-5-(5-((((R)-2-((R)-1-(N-((2-methyl-4-
(morpholinomethyl)benzoyl)oxy)formamido)propyl)heptanamido)methyl)carbamoyl)furan-2-
yl)phenyl)phosphonic acid
(3-ethoxy-5-(5-(((4R,5R)-5-ethyl-6-formyl-10-methyl-3,8-dioxo-4-pentyl-7-oxa-2,6,9-
triazaundecyl)carbamoyl)furan-2-yl)phenyl)phosphonic acid
(S)-2-(2-ethoxy-4-(5-((((R)-2-((R)-1-(N-((2-isopropyl-4-
methoxybenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-fluoro-2-
isopropylbenzoyl)oxy)formamido)propyl)heptanamido)methyl)carbamoyl)furan-2-
yl)benzamido)succinic acid
(3-ethoxy-5-(5-((((R)-2-((R)-1-(N-((4-fluoro-2-
isopropylbenzoyl)oxy)formamido)propyl)heptanamido)methyl)carbamoyl)furan-2-
yl)phenyl)phosphonic acid
(3-ethoxy-5-(5-((((R)-2-((R)-1-(N-
(nonanoyloxy)formamido)propyl)heptanamido)methyl)carbamoyl)furan-2-
yl)phenyl)phosphonic acid
(S)-2-(2-ethoxy-4-(5-((((R)-2-((R)-1-(N-((4-
(morpholinomethyl)benzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((2-methyl-4-
(morpholinomethyl)benzoyl)oxy)formamido)propyl)heptanamido)methyl)carbamoyl)furan-2-
yl)benzamido)succinic acid
(S)-2-(2-ethoxy-4-(5-(((4R,5R)-5-ethyl-6-formyl-10-methyl-3,8-dioxo-4-pentyl-7-oxa-2,6,9-
triazaundecyl)carbamoyl)furan-2-yl)benzamido)succinic acid
(3-ethoxy-5-(5-((((R)-2-((R)-1-(N-((4-
(morpholinomethyl)benzoyl)oxy)formamido)propyl)heptanamido)methyl)carbamoyl)furan-2-
yl)phenyl)phosphonic acid
(S)-2-(2-ethoxy-4-(5-(((4R,5R)-5-ethyl-6-formyl-10,10-dimethyl-3,8-dioxo-4-pentyl-7-oxa-
2,6,9-triazaundecyl)carbamoyl)furan-2-yl)benzamido)succinic acid
(S)-2-(2-ethoxy-4-(5-(((4R,5R)-5-ethyl-6-formyl-10-methyl-3,8-dioxo-4-pentyl-10-phenyl-7-
oxa-2,6,9-triazaundecyl)carbamoyl)furan-2-yl)benzamido)succinic acid
(S)-2-(2-ethoxy-4-(5-((((R)-2-((R)-1-(N-((3-(pyrrolidin-1-
yl)benzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((3-fluoro-4-(pyrrolidin-1-
yl)benzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-(2-oxopyrrolidin-1-
yl)benzoyl)oxy)formamido)propyl)heptanamido)methyl)carbamoyl)furan-2-
yl)benzamido)succinic acid
(S)-2-(4-(5-((((R)-2-((R)-1-(N-((4-(1H-pyrazol-1-
yl)benzoyl)oxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-
((phosphonooxy)methoxy)formamido)propyl)heptanamido)methyl)carbamoyl)furan-2-
yl)benzamido)succinic acid
(3-(5-((((R)-2-((R)-1-(N-((2,3-dihydrobenzo[b][1,4]dioxine-6-
carbonyl)oxy)formamido)propyl)heptanamido)methyl)carbamoyl)furan-2-yl)-5-
ethoxyphenyl)phosphonic acid
(3-ethoxy-5-(5-(((4R,5R)-5-ethyl-6-formyl-10,10-dimethyl-3,8-dioxo-4-pentyl-7,9-dioxa-2,6-
diazaundecyl)carbamoyl)furan-2-yl)phenyl)phosphonic acid
(S)-2-(4-(5-((((R)-2-((R)-1-(N-((2,3-dihydrobenzo[b][1,4]dioxine-6-
carbonyl)oxy)formamido)propyl)heptanamido)methyl)carbamoyl)furan-2-yl)-2-
ethoxybenzamido)succinic acid
(3-ethoxy-5-(5-((((R)-2-((R)-1-(N-((4-methyl-3,4-dihydro-2H-benzo[b][1,4]oxazine-6-
carbonyl)oxy)formamido)propyl)heptanamido)methyl)carbamoyl)furan-2-
yl)phenyl)phosphonic acid
(S)-2-(2-ethoxy-4-(5-((((R)-2-((R)-1-(N-((4-methyl-3,4-dihydro-2H-benzo[b][1,4]oxazine-6-
carbonyl)oxy)formamido)propyl)heptanamido)methyl)carbamoyl)furan-2-
yl)benzamido)succinic acid
(S)-2-(2-ethoxy-4-(5-(((4R,5R)-5-ethyl-6-formyl-10,10-dimethyl-3,8-dioxo-4-pentyl-7,9-dioxa-
2,6-diazaundecyl)carbamoyl)furan-2-yl)benzamido)succinic acid
(2S)-2-(2-(carboxymethoxy)-4-(5-((((R)-2-((R)-1-(N-((2-methoxy-6-
methylbenzoyl)oxy)formamido)propyl)heptanamido)methyl)carbamoyl)tetrahydrofuran-2-
yl)benzamido)succinic acid
(3-ethoxy-5-(5-((((R)-2-((R)-1-(N-((4-methoxy-2-
methylbenzoyl)oxy)formamido)propyl)heptanamido)methyl)carbamoyl)furan-2-
yl)phenyl)phosphonic acid
(3-ethoxy-5-(5-((((R)-2-((R)-1-(N-((2-isopropyl-4-
methoxybenzoyl)oxy)formamido)propyl)heptanamido)methyl)carbamoyl)furan-2-
yl)phenyl)phosphonic acid
(S)-2-(2-(carboxymethoxy)-4-(5-((((R)-2-((R)-1-(N-
((phenylcarbamoyl)oxy)formamido)propyl)heptanamido)methyl)carbamoyl)furan-2-
yl)benzamido)succinic acid
(S)-2-(4-(5-((((R)-2-((R)-1-(N-((3,4-
dimethoxybenzoyl)oxy)formamido)propyl)heptanamido)methyl)carbamoyl)furan-2-yl)-2-
ethoxybenzamido)succinic acid
(3-ethoxy-5-(5-((((R)-2-((R)-1-(N-
((phenylcarbamoyl)oxy)formamido)propyl)heptanamido)methyl)carbamoyl)furan-2-
yl)phenyl)phosphonic acid
(S)-2-(2-ethoxy-4-(5-((((R)-2-((R)-1-(N-((2-
methylbenzoyl)oxy)formamido)propyl)heptanamido)methyl)carbamoyl)furan-2-
yl)benzamido)succinic acid
(S)-2-(2-ethoxy-4-(5-(((4R,5R)-5-ethyl-6-formyl-11,11-dimethyl-3,10-dioxo-4-pentyl-7,9-
dioxa-2,6-diazadodecyl)carbamoyl)furan-2-yl)benzamido)succinic acid
N-{[2-(carboxymethoxy)-4-(5-{[(4R,5R)-5-ethyl-6-formyl-9,12,12-trimethyl-3,8,10-trioxo-4-
pentyl-7,11-dioxa-2,6,9-triazatridec-1-yl]carbamoyl}furan-2-yl)phenyl]carbonyl}-L-aspartic
acid
(S)-2-(4-(5-((((R)-2-((R)-1-(N-((2,4-
dimethoxybenzoyl)oxy)formamido)propyl)heptanamido)methyl)carbamoyl)furan-2-yl)-2-
ethoxybenzamido)succinic acid
(3-ethoxy-5-(5-((((R)-2-((R)-1-(N-((2-methyl-4-
morpholinobenzoyl)oxy)formamido)propyl)heptanamido)methyl)carbamoyl)furan-2-
yl)phenyl)phosphonic acid
(S)-2-(2-ethoxy-4-(5-((((R)-2-((R)-1-(N-((2-
isopropylbenzoyl)oxy)formamido)propyl)heptanamido)methyl)carbamoyl)furan-2-
yl)benzamido)succinic acid
(S)-2-(4-(5-((((R)-2-((R)-1-(N-((2,4-
dimethylnicotinoyl)oxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-((4-fluoro-2-
methylbenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((piperidine-4-
carbonyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-
(trifluoromethyl)benzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-
nitrobenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-
hydroxybenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((2-fluoro-4-
methoxybenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-methoxy-2-
(trifluoromethyl)benzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-methoxy-2-
(trifluoromethoxy)benzoyl)oxy)formamido)propyl)heptanamido)methyl)carbamoyl)furan-2-
yl)benzamido)succinic acid
((4-(5-((((R)-2-((R)-1-(N-
(benzoyloxy)formamido)propyl)heptanamido)methyl)carbamoyl)furan-2-yl)-2-
ethoxybenzamido)methyl)phosphonic acid
(S)-2-(2-ethoxy-4-(5-((((R)-2-((R)-1-(N-((2-methyl-4-
(trifluoromethyl)benzoyl)oxy)formamido)propyl)heptanamido)methyl)carbamoyl)furan-2-
yl)benzamido)succinic acid
(S)-2-(4-(5-((((R)-2-((R)-1-(N-((4-(4H-1,2,4-triazol-4-
yl)benzoyl)oxy)formamido)propyl)heptanamido)methyl)carbamoyl)furan-2-yl)-2-
ethoxybenzamido)succinic acid
(S)-2-(4-(5-((((R)-2-((R)-1-(N-((3-(1H-pyrrol-1-
yl)benzoyl)oxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-((4-(N-
methylmethylsulfonamido)benzoyl)oxy)formamido)propyl)heptanamido)methyl)carbamoyl)furan-
2-yl)benzamido)succinic acid
(S)-2-(4-(5-((((R)-2-((R)-1-(N-((4-(1H-pyrazol-1-
yl)benzoyl)oxy)formamido)propyl)heptanamido)methyl)carbamoyl)furan-2-yl)-2-
ethoxybenzamido)succinic acid
(S)-2-(4-(5-((((R)-2-((R)-1-(N-((4-(1H-imidazol-1-
yl)benzoyl)oxy)formamido)propyl)heptanamido)methyl)carbamoyl)furan-2-yl)-2-
ethoxybenzamido)succinic acid
((2-ethoxy-4-(5-((((R)-2-((R)-1-(N-((4-
methoxybenzoyl)oxy)formamido)propyl)heptanamido)methyl)carbamoyl)furan-2-
yl)benzamido)methyl)phosphonic acid
((2-ethoxy-4-(5-((((R)-2-((R)-1-(N-((4-methoxy-2-
methylbenzoyl)oxy)formamido)propyl)heptanamido)methyl)carbamoyl)furan-2-
yl)benzamido)methyl)phosphonic acid
((2-ethoxy-4-(5-((((R)-2-((R)-1-(N-((2-methyl-4-
(morpholinomethyl)benzoyl)oxy)formamido)propyl)heptanamido)methyl)carbamoyl)furan-2-
yl)benzamido)methyl)phosphonic acid
(S)-2-(2-ethoxy-4-(5-((((R)-2-((R)-1-(N-((4-(2-(2-
hydroxyethoxy)ethoxy)benzoyl)oxy)formamido)propyl)heptanamido)methyl)carbamoyl)furan-
2-yl)benzamido)succinic acid
(4R,5R)-4-ethyl-3-formyl-6,10-dioxo-5-pentyl-10-(5-phenylfuran-2-yl)-2-oxa-3,7,9-triazadecyl
dihydrogen phosphate
(S)-2-(2-ethoxy-4-(5-((((R)-2-((R)-1-(N-((2-methyl-6-
(phosphonooxy)benzoyl)oxy)formamido)propyl)heptanamido)methyl)carbamoyl)furan-2-
yl)benzamido)succinic acid
4-(5-((((R)-2-((R)-1-(N-(benzoyloxy)formamido)propyl)heptanamido)methyl)carbamoyl)furan-
2-yl)benzoic acid
(S)-2-(2-ethoxy-4-(5-((((R)-2-((R)-1-(N-
((phenylcarbamoyl)oxy)formamido)propyl)heptanamido)methyl)carbamoyl)furan-2-
yl)benzamido)succinic acid
(S)-2-(4-(5-((((R)-2-((R)-1-(N-((2-bromo-4-
methoxybenzoyl)oxy)formamido)propyl)heptanamido)methyl)carbamoyl)furan-2-yl)-2-
ethoxybenzamido)succinic acid
(S)-2-(4-(5-((((R)-2-((R)-1-(N-((2-chloro-4-
methoxybenzoyl)oxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-((4-
(methylamino)benzoyl)oxy)formamido)propyl)heptanamido)methyl)carbamoyl)furan-2-
yl)benzamido)succinic acid
(S)-2-(4-(5-((((R)-2-((R)-1-(N-((4-
aminobenzoyl)oxy)formamido)propyl)heptanamido)methyl)carbamoyl)furan-2-yl)-2-
ethoxybenzamido)succinic acid
(S)-2-(4-(5-((((R)-2-((R)-1-(N-((4-
(aminomethyl)benzoyl)oxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-((2-methyl-4-
nitrobenzoyl)oxy)formamido)propyl)heptanamido)methyl)carbamoyl)furan-2-
yl)benzamido)succinic acid
(S)-2-(4-(5-((((R)-2-((R)-1-(N-((3-
aminobenzoyl)oxy)formamido)propyl)heptanamido)methyl)carbamoyl)furan-2-yl)-2-
ethoxybenzamido)succinic acid
(S)-2-(4-(5-((((R)-2-((R)-1-(N-((3-
(dimethylamino)benzoyl)oxy)formamido)propyl)heptanamido)methyl)carbamoyl)furan-2-yl)-
2-ethoxybenzamido)succinic acid; and
(S)-2-(2-ethoxy-4-(5-((((R)-2-((R)-1-(N-((3-
(methylamino)benzoyl)oxy)formamido)propyl)heptanamido)methyl)carbamoyl)furan-2-
yl)benzamido)succinic acid

or a salt (e.g., a pharmaceutically acceptable salt) thereof.

In some embodiments, the compound of the invention is a compound of Formula (II) selected from the group consisting of:

(S)-2-(4-(5-((((R)-2-((R)-1-(N-
(benzoyloxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-((4-
methoxybenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-methoxy-2-
methylbenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-(2-oxopyrrolidin-1-
yl)benzoyl)oxy)formamido)propyl)heptanamido)methyl)carbamoyl)furan-2-
yl)benzamido)succinic acid
(S)-2-(4-(5-((((R)-2-((R)-1-(N-((4-(1H-pyrazol-1-
yl)benzoyl)oxy)formamido)propyl)heptanamido)methyl)carbamoyl)furan-2-yl)-2-
ethoxybenzamido)succinic acid; and
(S)-2-(2-ethoxy-4-(5-((((R)-2-((R)-1-(N-
((phosphonooxy)methoxy)formamido)propyl)heptanamido)methyl)carbamoyl)furan-2-
yl)benzamido)succinic acid

or a salt (e.g., a pharmaceutically acceptable salt) thereof.

In some embodiments, the invention is directed to a method of inhibiting BMP1, TLL1 and/or TLL2 comprising contacting a biological material comprising the protein(s) with a compound of the invention, particularly a compound of Formula (I) or (II), or a pharmaceutically acceptable salt thereof, or a composition formed by combining a compound of the invention and at least one excipient (e.g., a pharmaceutically acceptable excipient). In some embodiments the contact is made in-vitro, and the biological material is, e.g., cell culture or cellular tissue. In other embodiments, the contact is made in-vivo.

In other embodiments, the invention is directed to a method of treating a disease associated with BMP1, TLL1 and/or TLL2 activity in a subject (e.g., a human or other mammal) in need thereof, comprising administering to the subject a therapeutically effective amount of a compound of the invention (particularly a compound of Formula (I) or (II), or a pharmaceutically acceptable salt thereof), a pharmaceutical composition comprising the compound, or a pharmaceutical composition formed by combining the compound with one or more pharmaceutically acceptable excipients. The invention is still further directed to the use of a compound of the invention or a pharmaceutical composition comprising a compound of the invention (particularly a compound of Formula (I) or (II), or a pharmaceutically acceptable salt thereof), or formed by combining the compound with one or more pharmaceutically acceptable excipients, to treat a disease associated with BMP1, TLL1 and/or TLL2 activity. The invention is further directed to a compound of the invention ((particularly a compound of Formula (I) or (II), or a pharmaceutically acceptable salt thereof), or a composition formed by combining a compound of the invention with at least one excipient, for use in therapy, particularly as an active therapeutic substance in the treatment of a disease associated with BMP1, TLL1 and/or TLL2 activity. The invention is further directed to the use of a compound of the invention (particularly a compound of Formula (I) or (II), or a pharmaceutically acceptable salt thereof), or a composition formed by combining a compound of the invention with an excipient, in the manufacture of a medicament for use in treating a disease associated with BMP1, TLL1 and/or TLL2 activity.

In some embodiments the compound of Formula (I) or (II) is active in the treatment of the disease. In other embodiments, the compound of Formula (I) or (II) is converted to a compound which is active in the treatment of the disease (e.g., converted to a compound disclosed in PCT application no. PCT/IB2015/050179 or PCT publication no. WO2015/104684).

In some embodiments, the disease associated with BMP1, TLL1 and/or TLL2 activity is selected from those associated with pathological fibrotic conditions in body organs or tissues, e.g., such conditions of the heart, lung, kidney, liver, eye, skeletal muscle, skin, the vasculature, and the nervous system, e.g., myocardial infarction (“MI”), heart failure (e.g., heart failure with reduced ejection fraction, heart failure with preserved ejection fraction), cardiac arrhythmias (e.g., atrial fibrillation), hypertrophic cardiomyopathy, chronic obstructive pulmonary disease (“COPD”), idiopathic pulmonary fibrosis (“IPF”), diabetic nephropathy, post-acute kidney injury, chronic kidney disease (“CKD”), delayed graft function post-transplantation, liver cirrhosis, non-alcoholic steatohepatitis (“NASH”), glaucoma, corneal scarring, muscular dystrophies (including Duchenne, Becker, limb-girdle, congenital, facioscapulohumeral, myotonic, oculopharyngeal, distal, and Emery-Dreifuss), keloids, wound healing, adhesions, hypertrophic scarring and other scarring, e.g., associated with burns, surgery or other trauma, stroke, collagen vascular diseases (such as systemic lupus erythematosus, rheumatoid arthritis and scleroderma), spinal cord injury, and multiple sclerosis.

In some embodiments, the disease associated with BMP1, TLL1 and/or TLL2 activity is selected from muscular diseases characterized by reduced muscle function and/or mass, e.g., muscular dystrophy (e.g., Duchenne, Becker, limb-girdle, congenital, facioscapulohumeral, myotonic, oculopharyngeal, distal, and Emery-Dreifuss), sarcopenia, and cachexia associated with, e.g., heart failure, CKD, COPD, cancer, or old age.

The compounds according to Formula (I) and (II) may contain one or more asymmetric center(s) (also referred to as a chiral center(s)) and may, therefore, exist as individual enantiomers, diastereomers, or other stereoisomeric forms, or as mixtures thereof. Chiral centers, such as a chiral carbon, sulfur or phosphorus, may also be present in the compounds of this invention. Where the stereochemistry of a chiral center present in a compound of this invention (e.g., compound name or in any chemical structure illustrated herein) is not specified, the compound, compound name, or structure is intended to encompass all individual stereoisomers and all mixtures thereof. Thus, compounds according to Formula (I) and (II) containing one or more chiral center(s) may be present as racemic mixtures, enantiomerically enriched mixtures, or as enantiomerically pure individual stereoisomers.

Individual stereoisomers of a compound according to Formula (I) or (II) which contain one or more asymmetric center(s) may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

It is to be understood that a solid form of a compound of the invention may exist in crystalline forms, non-crystalline forms or a mixture thereof. Such crystalline forms may also exhibit polymorphism (i.e. the capacity to occur in different crystalline forms). These different crystalline forms are typically known as “polymorphs.” Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

Because of their potential use in medicine, the salts of the compounds of Formula (I) and (II) are preferably pharmaceutically acceptable salts. Suitable pharmaceutically acceptable salts include those described by Berge, S. M. et al., Journal of Pharmaceutical Sciences, 1977, 66, 1-19.

When a compound of the invention is a base (contains a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, and the like, or with a pyranosidyl acid, such as glucuronic acid or galacturonic acid, or with an alpha-hydroxy acid, such as citric acid or tartaric acid, or with an amino acid, such as aspartic acid or glutamic acid, or with an aromatic acid, such as benzoic acid or cinnamic acid, or with a sulfonic acid, such as p-toluenesulfonic acid, methanesulfonic acid, ethanesulfonic acid or the like. These acids may be used to provide negative counterions in compounds of of Formula (I) or (II) having a positively charged nitrogen atom.

Suitable acid addition salts include acetate, p-aminobenzoate, ascorbate, aspartate, benzenesulfonate, benzoate, bicarbonate, bismethylenesalicylate, bisulfate, bitartrate, borate, calcium edetate, camsylate, carbonate, clavulanate, citrate, cyclohexylsulfamate, edetate, edisylate, estolate, esylate, ethanedisulfonate, ethanesulfonate, formate, fumarate, gluceptate, gluconate, glutamate, glycollate, glycollylarsanilate, hexylresorcinate, hydrabamine, hydrobromide, hydrochloride, dihydrochloride, hydrofumarate, hydrogen phosphate, hydroiodide, hydromaleate, hydrosuccinate, hydroxynaphthoate, isethionate, itaconate, lactate, lactobionate, laurate, malate, maleate, mandelate, mesylate, methylsulfate, monopotassium maleate, mucate, napsylate, nitrate, N-methylglucamine, oxalate, oxaloacetate, pamoate (embonate), palmate, palmitate, pantothenate, phosphate/diphosphate, pyruvate, polygalacturonate, propionate, saccharate, salicylate, stearate, subacetate, succinate, sulfate, tannate, tartrate, teoclate, tosylate, triethiodide, trifluoroacetate and valerate.

Other exemplary acid addition salts include pyrosulfate, sulfite, bisulfite, decanoate, caprylate, acrylate, isobutyrate, caproate, heptanoate, propiolate, oxalate, malonate, suberate, sebacate, butyne-1,4-dioate, hexyne-1,6-dioate, chlorobenzoate, methylbenzoate, dinitrobenzoate, hydroxybenzoate, methoxybenzoate, phthalate, phenylacetate, phenylpropionate, phenylbutrate, lactate, γ-hydroxybutyrate, mandelate, and sulfonates, such as xylenesulfonate, propanesulfonate, naphthalene-1-sulfonate and naphthalene-2-sulfonate.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound.

When a compound of the invention is an acid (contains an acidic moiety), a desired salt may be prepared by any suitable method known to the art, including treatment of the free acid with an inorganic or organic base, such as an amine (primary, secondary, tertiary or quaternary), an alkali metal or alkaline earth metal hydroxide, alkoxide (e.g. (C₁₋₄)alkoxide), alkyl ester (e.g., (C₁₋₄)alkyl ester, e.g. acetate), or the like. Illustrative examples of suitable salts include organic salts derived from amino acids such as glycine, lysine, and arginine, ammonia, primary, secondary, tertiary, and quaternary amines, cyclic amines, and amino sugars, e.g., 2-amino-2-deoxysugars, such as N-methyl-D-glucamine, diethylamine, isopropylamine, trimethylamine, ethylene diamine, dicyclohexylamine, ethanolamine, choline, piperidine, morpholine, piperazine, Tris (also known as THAM, or tris(hydroxymethyl)aminomethane), 2-amino-2-hydroxymethyl-propane-1,3-diol, and 2-amino-2-(hydroxymethyl)-1,3-propanediol), meglumine (also known as 1-Deoxy-1-(methylamino)-D-glucitol), galactosamine, glucosamine, and N-acetylglucosamine, as well as inorganic salts derived from sodium, calcium, potassium, magnesium, manganese, iron, copper, zinc, aluminum, and lithium (e.g., hydroxides, (C₁₋₄)alkoxides, and (C₁₋₄)alkyl esters of such alkali and alkaline earth metals).

Treatment of a compound of Formula (I) or (II) containing a free acid with an inorganic or organic base, or containing a free base with an acid, to form a salt of the compound of Formula (I) or (II) may be done by methods known in the art. For example, the free acid may be admixed with a suitable solvent (e.g. in which the free acid is soluble) and treated with the base, with stirring, and optionally with heating and/or temperature cycling. Analogously, for a compound of Formula (I) or (II) containing a free base, the free base may be admixed with a suitable solvent (e.g. in which the free base is soluble) and treated with the acid, with stirring, and optionally with heating and/or temperature cycling. Certain of the compounds of the invention may form salts with one or more equivalents of an acid (if the compound contains a basic moiety) or a base (if the compound contains an acidic moiety). The present invention includes within its scope all possible stoichiometric and non-stoichiometric salt forms.

Compounds of the invention having both a basic and acidic moiety may be in the form of zwitterions, acid-addition salts of the basic moiety or base salts of the acidic moiety.

This invention also provides for the conversion of one pharmaceutically acceptable salt of a compound of this invention into another pharmaceutically acceptable salt of a compound of this invention.

The compounds of Formula (I) and (II), and salts (including pharmaceutically acceptable salts) thereof may be in the form of a solvate. For solvates of the compounds of Formula (I) and (II), including solvates of salts of the compounds of Formula (I) and (II), that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, dimethylsulfoxide, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Solvates include stoichiometric solvates as well as compositions containing variable amounts of the incorporated solvent(s), e.g. a hydrate includes stoichiometric hydrates and compositions containing variable amounts of water. The invention includes all such solvates, particularly hydrates. It is to be understood that the term “a salt, particularly a pharmaceutically acceptable salt, thereof, or solvate thereof” and the like in reference to a compound of Formula (I) or (II) encompasses respectively a salt of a compound of Formula (I) or (II), a pharmaceutically acceptable salt of a compound of Formula (I) or (II), a solvate of a compound of Formula (I) or (II), a solvate of a salt of a compound of Formula (I) or (II), and a solvate of a pharmaceutically acceptable salt of a compound of Formula (I) or (II) (for example, where water is the incorporated solvent, said solvates are hydrates).

Because the compounds of the invention, particularly compounds of Formula (I) and (II), and pharmaceutically acceptable salts thereof, or a solvate (e.g., hydrate) thereof, are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

### General Methods of Preparation

The compounds of Formula (I) and (II) may be obtained by using synthetic procedures illustrated in the Schemes below or by drawing on the knowledge of a skilled organic chemist. The syntheses provided in these Schemes are applicable for producing compounds of the invention having a variety of different R1, R2, R3 and as applicable R4 groups employing appropriate precursors. Those skilled in the art will appreciate that in the preparation of compounds of the invention it may be necessary and/or desirable to protect one or more sensitive groups in the molecule or the appropriate intermediate to prevent undesirable side reactions. Suitable protecting groups for use according to the present invention are well know to those skilled in the art and may be used in a conventional manner. See for example, “Protective groups in organic synthesis” by T. W. Green and P. G. M Wuts (Wiley & Sons, 1991) or “Protecting Groups” by P. J. Kocienski (Georg Thieme Verlag, 1994). Subsequent deprotection, where needed, affords compounds of the nature generally disclosed. While the Schemes are shown with compounds of Formula (I) and (II), they are illustrative of processes that may be used to make the compounds of the invention.

Compound names were generated using the software naming program Chem Draw Ultra v12.0 available from Perkin Elmer, 940 Winter Street, Waltham, Mass., 02451, USA. (http://www.perkinelmer.com/). Example 95 and the preceding intermediate were named using Advanced Chemistry Development, Inc. ACD/Labs Release: 11:00 Product Version 11.01 (Build 22379, 18 Oct. 2007) available from Advanced Chemistry Development, Inc. 110 Yonge Street 14^{th} Floor Toronto, Ontario M5C 1T4, Canada.

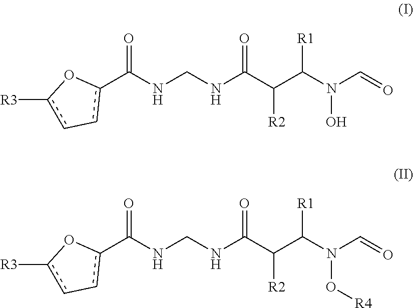

In a general process, compounds of Formula (I) may be prepared according to reaction Schemes 1:

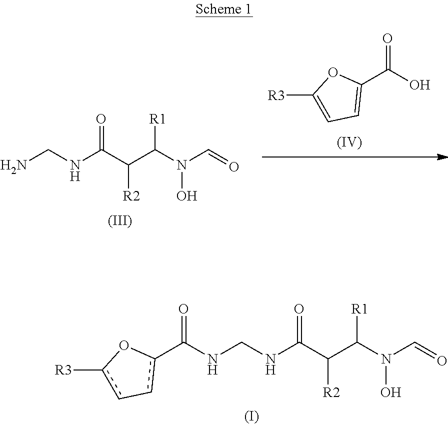

React (III) and (IV) in the presence of an amide coupling reagent (e.g. EDC/HOBT, HATU or HBTU, iPrOC(O)Cl, iBuOC(O)Cl, EtOC(O)Cl) in the presence of a base (e.g. TEA, DIPEA or NMO) in a solvent such as DCM or DMF in the presence or absence of TMSCl at room temperature or at an elevated temperature such as 50 OC to yield the final product (I).

In a general process, compounds of Formula (III) may be prepared according to reaction Scheme 2.

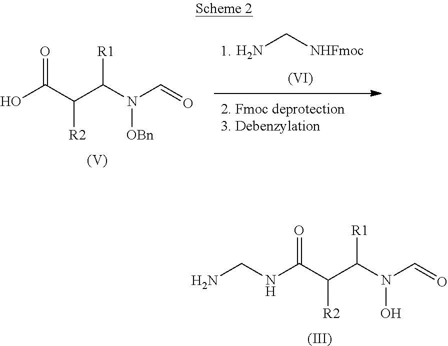

1. React compound of Formula (V) with (VI) in the presence of a coupling reagent such as HATU in the presence of a base such as DIPEA in a suitable solvent such as DMF at room temperature. 2. Fmoc deprotection may be achieved by reaction with a secondary amine such as morpholine in a suitable solvent such as acetonitrile. 3. Debenzylation may be achieved via hydrogenation using a catalyst such as Pd/C and a hydrogen source (e.g. hydrogen gas or ammonium formate) at atmospheric pressure and temperature.

With regard to the above Schemes 1-2:

Compounds of Formula (IV) are commercially available or may be prepared by methods known in the literature or by processes known to those skilled in the art.

Compound (VI) may be prepared by processes known to those skilled in the art.

In a general process, compounds of Formula (V) may be prepared according to reaction Schemes 3:

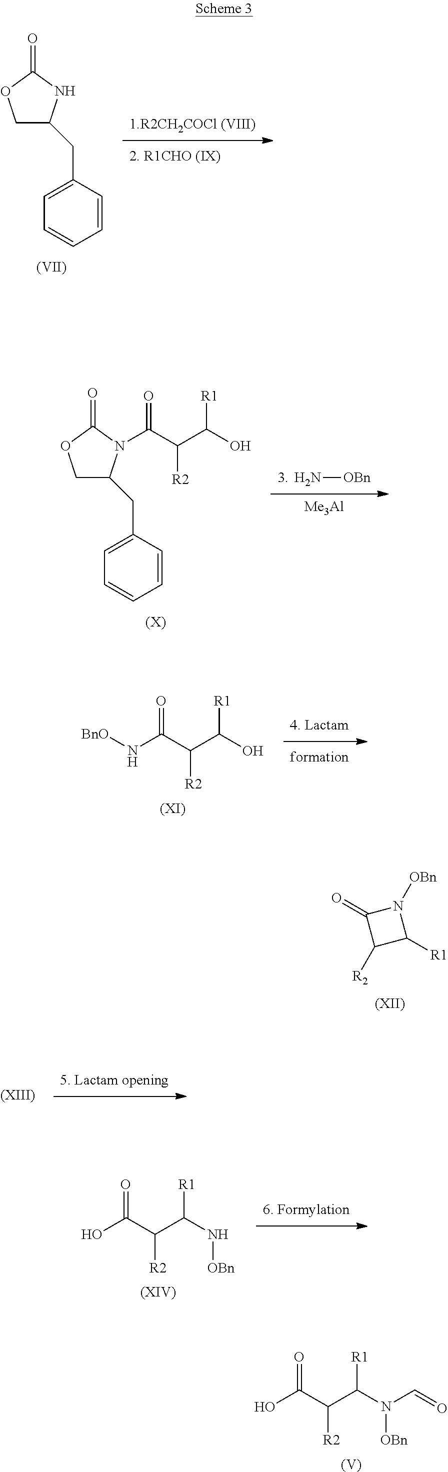

1. React the appropriate enantiomer of (VII) with a base such as butyl lithium followed by the appropriate acyl chloride (VIII) in a suitable solvent such as THF. 2. React with TiCl₄ in the presence of a suitable base such as DIPEA with a suitable additive such as NMP in a suitable solvent such as DCM followed by reaction with the appropriate aldehyde (IX). 3. React with O-benzylhydroxylamine hydrochloride in a suitable solvent such as THF in the presence of trimethylaluminium. 4. React with methanesulfonyl chloride using a suitable base such as pyridine as the solvent. 5. React with tetrabutylammonium hydroxide in a suitable solvent such as 2-methyl tetrahydrofuran. 6. Formylation may be achieved utilizing a mixture of CDI/formic acid in a solvent such as DCM at room temperature.

In a general process, compounds of Formula (II) may be prepared according to reaction Schemes 4:

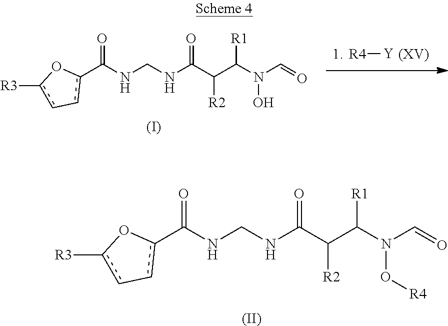

1. React (I) with (XV) in the presence of a suitable base (Et₃N, DIPEA, K₂CO₃) in a solvent (THF, MeCN, DMF).

With regard to the above Scheme 4:

Compounds (XV) are commercially available, may be prepared by methods known in the literature or by processes known to those skilled in the art. Y may be defined as a halogen, appropriate anhydride, or other leaving group.

In a general process, compounds of Formula (II) wherein R3 contains a carboxylic acid (e.g., Formula (IIb)) or phosphonic acid (e.g., Formula (IId) or (IIf)) may be prepared according to Schemes 1-4 above from their corresponding ester functionalities (IIa) and (IIc) or (lie) as shown in scheme 5. The transformations in Scheme 5 are illustrated with a phenyl ring R3 however Scheme 5 applies analogously to preparation of corresponding molecules of Formula (IIa-b) with all embodiments of R3 disclosed herein (including, e.g., where R3 is heteroaryl and/or optionally further substituted).

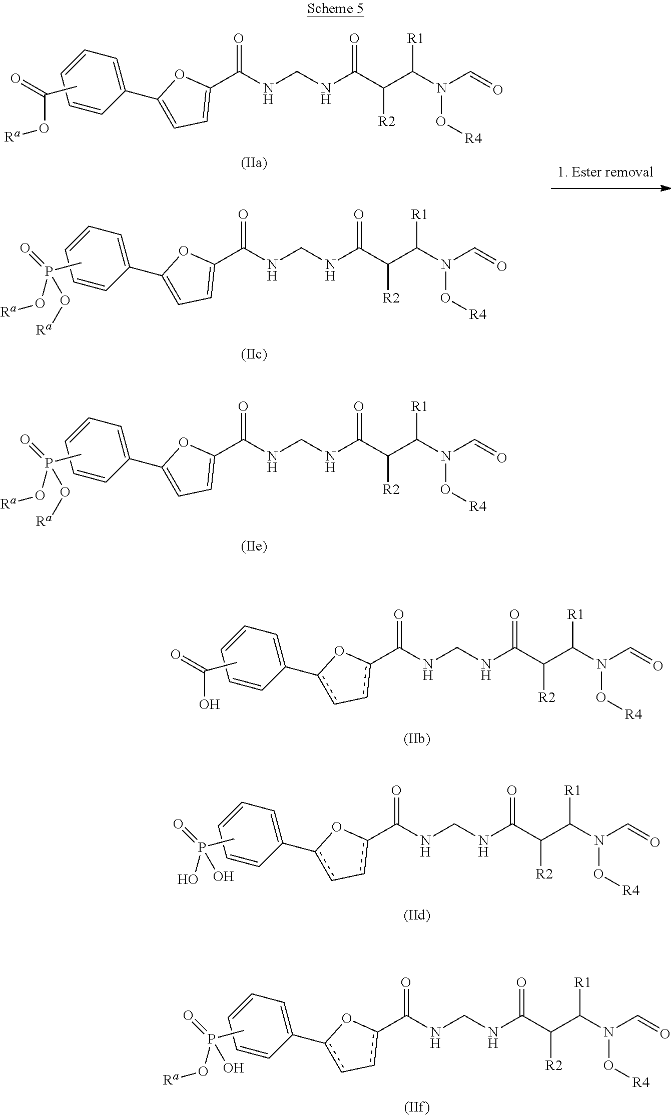

1. Ester removal may include the following: Debenzylation may be achieved as described in Step 3 of Scheme 2. Ester hydrolysis may be achieved by reaction with lithium or sodium hydroxide in a suitable solvent such as a THF/water mixture or alcoholic solvent (e.g. EtOH or MeOH/water mixture). For compounds of Formula (IIc), hydrolysis may be alternatively achieved by reaction with TMS-Br in a suitable solvent such as DCM at 0° C. to room temperature. Additionally, ester hydrolysis may be achieved by treatment with TFA in a suitable solvent such as DCM.

Further details for the preparation of compounds of the invention are found in the Intermediates and Examples section hereinafter.

### Use of Compounds of the Invention

The compounds of the invention are BMP1, TLL1 and/or TLL2 inhibitors, or reveal (convert to or generate) a BMP1, TLL1 and/or TLL2inhibitor in vivo, ex vivo or in vitro. In some embodiments a compound of the invention is both itself a BMP1, TLL1 and/or TLL2 inhibitor, and reveals a different BMP1, TLL1 and/or TLL2 inhibitor in vivo, ex vivo or in vitro. In some embodiments the compound is a derivative of a BMP1, TLL1 and/or TLL2 inhibitor disclosed in PCT application no. PCT/IB2015/050179 or PCT publication no. WO2015/104684. In some embodiments, the compound reveals a BMP1, TLL1 and/or TLL2 inhibitor disclosed in PCT application no. PCT/IB2015/050179 or PCT publication no. WO2015/104684.

The compounds of the invention may generate a BMP1, TLL1 and/or TLL2 inhibitor under one or more conditions, for example:
a) the compound of the invention may generate a BMP1, TLL1 and/or TLL2 inhibitor in vivo following administration to a subject: e.g., in plasma (e.g. following injection), or after oral administration; and/or the compound of the invention may generate a BMP1, TLL1 and/or TLL2 inhibitor ex vivo in isolated plasma (which may be used to predict in vivo generation of the BMP1, TLL1 and/or TLL2inhibitor, e.g. following injection). Such compounds of the invention may be described as “pro-drugs”, and/or
b) the compound of the invention may generate a BMP1, TLL1 and/or TLL2 inhibitor when combined with one or more suitable excipients, e.g. to reveal the BMP1, TLL1 and/or TLL2 inhibitor before administration to a subject. For example, the compound of the invention may be combined with a solution of suitable pH which may contain an agent which promotes generation of the BMP1, TLL1 and/or TLL2 inhibitor, e.g. an enzyme on a bead, a functionalized resin, etc.) Such compounds of the invention may be described as “pre-drugs”.

Conversion of a pro-drug or pre-drug to the BMP1, TLL1 and/or TLL2 inhibitor may be assessed, respectively, using the plasma stability assay or solution stability assay described herein. Conversion of a prodrug to the BMP1, TLL1 and/or TLL2 inhibitor in vivo following administration to a subject may be determined by standard pharmacokinetic techniques.

Accordingly, the compounds of this invention may be particularly useful for treatment of diseases associated with BMP1, TLL1 and/or TLL2 activity, including for example treatment of diseases where inhibition of BMP1, TLL1 and/or TLL2 is of therapeutic benefit. For example, compounds of the invention may be particularly useful for treatment of diseases where inhibition of tissue ECM (extracellular matrix) production and/or maturation would be beneficial, or where inhibition of myostatin activity would be beneficial.

In some embodiments, the disease associated with BMP1, TLL1 and/or TLL2 activity is selected from diseases associated with pathological fibrotic conditions in body organs or tissues, e.g., such conditions of the:

heart (e.g., myocardial infarction (“MI”), heart failure (e.g., heart failure with reduced ejection fraction, heart failure with preserved ejection fraction), cardiac arrhythmias (e.g., atrial fibrillation), hypertrophic cardiomyopathy),

lung (e.g. chronic obstructive pulmonary disease (“COPD”), idiopathic pulmonary fibrosis (“IPF”)),

kidney (e.g. diabetic nephropathy, post-acute kidney injury, chronic kidney disease (“CKD”), delayed graft function post-transplantation),

liver (e.g. liver cirrhosis, non-alcoholic steatohepatitis (“NASH”)),

eye (e.g. glaucoma, corneal scarring),

skeletal muscle (e.g. muscular dystrophies, including Duchenne, Becker, limb-girdle, congenital, facioscapulohumeral, myotonic, oculopharyngeal, distal, and Emery-Dreifuss),

skin (e.g. keloids, wound healing, adhesions, hypertrophic scarring and other scarring, e.g., associated with burns, surgery or other trauma),

the vasculature (e.g. stroke, and collagen vascular diseases such as systemic lupus erythematosus, rheumatoid arthritis and scleroderma), and

the nervous system (e.g. spinal cord injury, multiple sclerosis).

In some embodiments, the disease associated with BMP1, TLL1 and/or TLL2 activity is selected from muscular diseases characterized by reduced muscle function and/or mass, e.g., muscular dystrophy (e.g., Duchenne, Becker, limb-girdle, congenital, facioscapulohumeral, myotonic, oculopharyngeal, distal, and Emery-Dreifuss), sarcopenia, and cachexia associated with, e.g., heart failure, CKD, COPD, cancer, or old age.

Accordingly, this invention provides a method of treating a disease associated with BMP1, TLL1 and/or TLL2 activity in a subject in need thereof (e.g. a human or other mammal, particularly a human), for example the diseases recited herein, comprising administering to the subject a therapeutically effective amount of a compound of Formula (I) or (II), or a salt thereof, particularly a pharmaceutically acceptable salt thereof.

In some embodiments, a compound of the invention is administered post-MI (i.e. to a subject who has suffered an MI), e.g. to treat fibrosis associated with myocardial infarction. In some embodiments, a compound of the invention is administered post-MI, e.g. to prevent fibrosis associated with myocardial infarction.

In some embodiments, the method of treating comprises administering a specific compound described herein, e.g., a compound of the Examples, or any alternative stereoisomeric form, free acid/base form, salt form, or alternative salt form (particularly pharmaceutically acceptable salts or alternative pharmaceutically acceptable salt forms) thereof, as applicable.

This invention also provides a compound of Formula (I) or (II), or a salt thereof, particularly a pharmaceutically acceptable salt thereof, for use in therapy. This invention specifically provides for the use of a compound of Formula (I) or (II), or a pharmaceutically acceptable salt thereof, as an active therapeutic substance in the treatment of a disease associated with BMP1, TLL1 and/or TLL2 activity, for example the diseases recited herein.

This invention specifically provides a compound of Formula (I) or (II), or a pharmaceutically acceptable salt thereof, for use as an active therapeutic substance in the treatment of a disease associated with BMP1, TLL1 and/or TLL2 activity, for example the diseases recited herein.

In some embodiments, the compound for use in therapy, e.g. for use in the treatment of a disease associated with BMP1, TLL1 and/or TLL2 activity, is a specific compound described herein, e.g., a compound of the Examples, or any alternative stereoisomeric form, free acid/base form, salt form, or alternative salt form (particularly pharmaceutically acceptable salts or alternative pharmaceutically acceptable salt forms) thereof, as applicable.

The invention also provides for the use of a compound of Formula (I) or (II), or a salt thereof, particularly a pharmaceutically acceptable salt thereof, in the manufacture of a medicament for use in the treatment of a disease associated with BMP1, TLL1 and/or TLL2 activity, for example the diseases recited herein.

In some embodiments, the invention provides for the use of a specific compound described herein, e.g., a compound of the Examples, or any alternative stereoisomeric form, free acid/base form, salt form, or alternative salt form (particularly pharmaceutically acceptable salts or alternative pharmaceutically acceptable salt forms) thereof, as applicable, in the manufacture of a medicament for use in the treatment of a disease associated with BMP1, TLL1 and/or TLL2 activity.

Treatment of a disease associated with BMP1, TLL1 and/or TLL2 activity may be achieved using a compound of this invention as a monotherapy, or in dual or multiple combination therapy. For example, the compounds of this invention may be administered in combination with one or more therapeutically active agents selected from the group consisting of: anticoagulants, angiotensin-converting-enzyme (ACE) inhibitors, angiotensin II receptor blockers (ARBs), beta(“β”)-blockers, aldosterone antagonists, diuretics, vasodilators, cholesterol-lowering drugs (e.g., statins, fibrates, niacin, resins), statins, platelet antagonists, anti-arrhythmics, calcium channel blockers, erythropoiesis-stimulating agents (ESAs), iron, beta agonists, inhaled or oral steroids, anticholinergics, theophylline, PDE4 inhibitors, antibiotics, other antifibrotic agents, PDE5 inhibitors, immune modulators, neprilysin inhibitors, and digitalis preparations, e.g., any such agents as are known in the art, and combinations thereof. Particular therapeutic agents in these classes include those in the United States Pharmacopeia (USP). It will be understood that a particular active agent may fall within one or more of the foregoing classes. Such agents may be administered in therapeutically effective amounts, e.g., as is known in the art, or lesser or greater amounts than known in the art provided that the amount administered is therapeutically effective.

For example, treatment of cardiac diseases may include administration of one or more agents selected from the group: anticoagulants, ACE inhibitors, ARBs, R-blockers, aldosterone antagonists, diuretics, vasodilators (e.g. nitrates), cholesterol lowering drugs (e.g., statins, fibrates, niacin, resins), platelet antagonists, anti-arrhythmics, calcium channel blockers, neprilysin inhibitors, digitalis preparations, and combinations thereof. In particular embodiments, treatment of atrial fibrillation, heart failure, or hypertrophic cardiomyopathy may comprise administration of one or more such agents.

As another example, treatment of CKD may include administration of one or more agents selected from ESAs, iron, ACE inhibitors, ARBs, R-blockers, diuretics, calcium channel blockers, statins, and combinations thereof.

In other exemplary embodiments, treatment of COPD may include administration of one or more agents selected from the group: beta agonists, inhaled or oral steroids, anticholinergics, theophylline, PDE4 inhibitors, antibiotics, and combinations thereof.

For example, idiopathic pulmonary fibrosis may include administration of one or more agents selected from the group: antifibrotics, PDE5 inhibitors, immune modulators, and combinations thereof.

Particular examples of other therapeutically active agents which may be used in combination with one or more compounds of the invention, for example to treat cardiac diseases, include:

anticoagulants such as: dalteparin (FRAGMIN), danaparoid (ORGARAN), enoxaparin (LOVENOX), heparin, tinzaparin (INNOHEP), warfarin (COUMADIN), alteplase, aspirin, ardeparin, fondaparinux, lepirudin, desirudin, bivalirudin, urokinase, rivaroxaban, apixaban, dabigatran, argatroban;

ACE inhibitors such as benazepril (LOTENSIN), captopril (CAPOTEN), enalapril (VASOTEC), fosinopril (MONOPRIL), lisinopril (PRINIVIL, ZESTRIL), moexipril (UNIVASC), perindopril (ACEON), quinapril (ACCUPRIL), Ramipril (ALTACE), trandolapril (MAVIK), imidapril;

ARBs such as candesartan (ATACAND), eprosartan (TEVETEN), irbesartan (AVAPRO), losartan (COZAAR), telmisartan (MICARDIS), valsartan (DIOVAN), olmesartan, azilsartan;

beta-blockers such as acebutolol (SECTRAL), atenolol (TENORMIN), betaxolol (KERLONE), bisoprolol/hydrochlorothiazide (ZIAC), bisoprolol (ZEBETA), carteolol (CARTROL), metoprolol (LOPRESSOR, TOPROL XL), nadolol (CORGARD), propranolol (INDERAL), sotalol (BETAPACE), timolol (BLOCADREN);

aldosterone antagonists such as spironolactone, eplerenone, Canrenone (canrenoate potassium), Prorenone (prorenoate potassium), Mexrenone (mexrenoate potassium);

diuretics such as amiloride (MIDAMOR), bumetanide (BUMEX), chlorothiazide (DIURIL), chlorthalidone (HYGROTON), furosemide (LASIX), hydro-chlorothiazide (ESIDRIX, HYDRODIURIL), indapamide (LOZOL), spironolactone (ALDACTONE), metolazone, torsemide, triamterene;

vasodilators such as nitroglycerin, isosorbide dinitrate (ISORDIL), isosorbide mononitrate, nesiritide (NATRECOR), hydralazine (APRESOLINE)

cholesterol-lowering drugs, e.g., statins, such as atorvastatin, fluvastatin, lovastatin, pitavastatin, pravastatin, rosuvastatin, simvastatin, including combination products, such as ADVICOR (lovastatin/niacin extended-release), SIMCOR (simvastatin/niacin extended-release), and VYTORIN (simvastatin/ezetimibe); nicotinic acid (niacin), fibrates such as gemfibrozil (LOPID), fenofibrate (TRICOR, FIBRICOR), clofibrate;

platelet antagonists such as aspirin, ticlopidine, clopidogrel (PLAVIX), dipyridamole;

anti-arrhythmics such as quinidine, procainamide, disopyramide, lidocaine, phenytoin, mexiletine, tocainide, encainide, flecainide, propafenone, moricizine, carvedilol, propranolol, esmolol, timolol, metoprolol, atenolol, bisoprolol, amiodarone, sotalol, ibutilide, dofetilide, dronedarone, verapamil, diltiazem, adenosine, digoxin, magnesium sulfate;

calcium channel blockers, such as amlodipine (NORVASC, LOTREL), bepridil (VASCOR), diltiazem (CARDIZEM, TIAZAC), felodipine (PLENDIL), nifedipine (ADALAT, PROCARDIA), nimodipine (NIMOTOP), nisoldipine (SULAR), verapamil (CALAN, ISOPTIN, VERELAN), isradipine, nicardipine;

neprilysin inhibitors such as sacubitril, including, e.g., a combination of sacubitril and valsartan, such as LCZ696;

digitalis preparations such as digoxin, digitoxin.

Combination therapy includes administration of the therapeutically active agents in separate dosage forms or together in a single dosage form. Combination therapy may involve simultaneous administration or separate administration of the therapeutically active agents, which may be substantially simultaneous or substantially separate administration. Typically, combination therapy will involve administration of each agent such that therapeutically effective amounts of each agent are present in the subject's body in at least an overlapping period.

In some embodiments, combination therapy comprises administering a specific compound described herein, e.g., a compound of the Examples, or any alternative stereoisomeric form, free acid/base form, salt form, or alternative salt form (particularly pharmaceutically acceptable salts or alternative pharmaceutically acceptable salt forms) thereof, as applicable, and one or more additional therapeutically active agents.

Accordingly, the present invention provides a composition comprising a) a compound of Formula (I) or (II), or a pharmaceutically acceptable salt thereof and b) a combination partner. As used herein, suitable combination partners include one or more other therapeutically active agents such as those described above by classification or more particularly.

The present invention further provides a method for treating a disease associated with BMP1, TLL1 and/or TLL2 activity in a subject (e.g. a human or other mammal, particularly a human) in need thereof comprising administering to said subject a therapeutically effective amount of a) a compound of Formula (I) or (II), or a pharmaceutically acceptable salt thereof and b) a combination partner. The individual components of the combination may be administered either sequentially or simultaneously in separate or combined pharmaceutical formulations by any convenient route.

The invention further provides a combination of a) a compound of Formula (I) or (II), or a pharmaceutically acceptable salt thereof and b) a combination partner.

In the compositions, methods and combinations of the invention comprising a combination partner, suitable combination partners include other therapeutically active agents such as described above by classification or more particularly.

In some embodiments of the compositions, methods and combinations of the inventions comprising a combination partner, the compound of Formula (I) or (II), or a pharmaceutically acceptable salt thereof is a specific compound described herein, e.g., a compound of the Examples, or any alternative stereoisomeric form, free acid/base form, pharmaceutically acceptable salt form or alternative pharmaceutically acceptable salt form thereof, as applicable.

A “therapeutically effective amount” is intended to mean that amount of a compound that, when administered to a subject in need of such treatment, is sufficient to effect treatment, as defined herein. Thus, for example, a therapeutically effective amount of a compound of the invention, e.g. a compound of Formula (I) or (II), or a pharmaceutically acceptable salt thereof, is a quantity of such agent that, when administered to a subject (e.g., human) in need thereof, is sufficient to modulate or inhibit the activity of BMP1, TLL1 and/or TLL2 such that a disease condition which is mediated or inhibited by that activity is reduced, alleviated or prevented. The amount of a given compound that will correspond to such an amount will vary depending upon factors such as the particular compound (e.g., the potency (pIC₅₀) and the biological half-life of the particular compound), disease condition and its severity, and the identity (e.g., age, size and weight) of the subject in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the subject in need of treatment (e.g., weight), the particular compound and its properties (e.g., pharmaceutical characteristics), disease and its severity and the specific composition and method being used, but can nevertheless be determined by one of skill in the art.

In some embodiments, 0.1 mg to 1000 mg (e.g., 0.1-500 mg, or 0.1-100 mg) of a compound of the invention, particularly a compound of Formula (I) or (II), or a pharmaceutically acceptable salt thereof, is administered at a frequency of twice a day, once a day, once a week, or frequencies therebetween. In some embodiments, a compound of the invention, particularly a compound of Formula (I) or (II), or a pharmaceutically acceptable salt thereof, is administered sub-cutaneously in an amount of less than 100 mg per dose (e.g., 0.1-<100 mg per dose).

“Treat”, “treating” or “treatment” is intended to mean at least the mitigation of a disease in a subject. The methods of treatment for mitigation of a disease include the use of the compounds in this invention in any conventionally acceptable manner, for example for prevention, retardation, prophylaxis, therapy, improvement or cure of a disease. Thus, treatment may involve at least the mitigation of one or more symptoms of a disease. Specific diseases that may be particularly susceptible to treatment using a compound of this invention include those described herein.

The compounds of the invention may be administered by any suitable route of administration, including both systemic administration and topical administration. Systemic administration includes oral administration, parenteral administration, transdermal administration, rectal administration, and administration by inhalation. Oral administration includes enteral (digestive tract) and buccal or sublingual administration. Parenteral administration refers to routes of administration other than enteral, transdermal, or by inhalation, and is typically by injection or infusion into tissue or blood. Parenteral administration includes intravenous, intramuscular, subcutaneous, intradermal, and transdermal implant injection or infusion. Inhalation refers to administration into the subject's lungs whether inhaled through the mouth or through the nasal passages. Topical administration includes application to the skin.

For use in therapy, the compounds of the invention will be normally, but not necessarily, formulated into a pharmaceutical composition prior to administration to a subject. Accordingly, the invention also is directed to pharmaceutical compositions comprising a compound of the invention, particularly a compound of Formula (I) or (II), or a pharmaceutically acceptable salt thereof, and one or more pharmaceutically acceptable excipients.

The pharmaceutical compositions of the invention may be prepared and packaged in bulk form wherein an effective amount of a compound of the invention can be extracted and then given to the subject such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of Formula (I) or (II), or a salt, particularly a pharmaceutically acceptable salt, thereof). When prepared in unit dosage form, the pharmaceutical compositions may contain from 0.1 mg to 1000 mg (e.g., 0.1-500 mg, or 0.1-100 mg) of a compound of this invention.

The pharmaceutical composition may include one or more compounds of the invention and/or one or more pharmaceutically acceptable excipients. The pharmaceutical compositions of the invention typically contain one compound of the invention. However, in certain embodiments, the pharmaceutical compositions of the invention contain more than one compound of the invention. In addition, the pharmaceutical compositions of the invention may optionally further comprise one or more additional pharmaceutically active compounds, e.g., the therapeutically active agents described above by classification or more particularly.

In some embodiments, the pharmaceutical composition comprises a) 0.01-100 mg of a compound of formula (I) of (II), or a pharmaceutically acceptable salt thereof and b) 0.001-900 mg of one or more pharmaceutically acceptable excipients. In some embodiments, the pharmaceutical composition comprises a) 0.01-100 mg/mL of a compound of formula (I) or (II), or a pharmaceutically acceptable salt thereof and b) 0.001-900 mg/mL of one or more pharmaceutically acceptable excipients.

In some embodiments, the pharmaceutical composition comprises a specific compound described herein, e.g., a compound of the Examples, or any alternative stereoisomeric form, free acid/base form, pharmaceutically acceptable salt form, or alternative pharmaceutically acceptable salt form thereof, as applicable.

As used herein, “pharmaceutically acceptable excipient” means a material, composition or vehicle other than a pharmaceutical active ingredient(s) intended for treating a disease (e.g., a compound of the invention). Pharmaceutically acceptable excipients are involved in providing a property or function useful to a pharmaceutical composition, for example an excipient may be involved in modifying physical, sensory, stability, or pharmaco-kinetic properties of the composition, for example in giving form or consistency to the composition, in bulking up the active ingredient (e.g. for convenient and accurate dispensation), in enhancing therapy (e.g. facilitating drug absorption or solubility, or other pharmacokinetic properties), in the manufacturing process (e.g. as a handling or processing aid), in stabilizing the composition, or in enhancing subject compliance (e.g., enhancing palatability or appearance of the composition). Each excipient must be compatible with the other ingredients of the pharmaceutical composition when commingled such that interactions which would substantially reduce the efficacy of the compound of the invention (or any other active ingredient, if present) when administered to a subject and interactions which would result in pharmaceutical compositions that are sufficiently high purity to render it pharmaceutically acceptable.

The compounds of the invention and the pharmaceutically acceptable excipient or excipients will typically be formulated into a dosage form adapted for administration to the subject by the desired route of administration. Conventional dosage forms include those adapted for (1) oral administration such as tablets, capsules, caplets, pills, lozenges, troches, powders, syrups, elixirs, suspensions, solutions, emulsions, sachets, and cachets; (2) parenteral administration such as sterile solutions, suspensions, lyophiles, microparticles, nanocarriers, implants, preformed implants and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, gels, dermal patches, and transdermal patches or sprays.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to: facilitate the production of uniform dosage forms, to facilitate the production of stable dosage forms, to facilitate the carrying or transporting the compound or compounds of the invention once administered to the subject from one organ, or portion of the body, to another organ, or portion of the body, and/or to enhance subject compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, carriers, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press), including current and past editions.

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising an effective amount of a compound of the invention and a diluent or filler, and optionally a binder, disintegrant, and/or lubricant. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). Suitable disintegrants include crospovidone, sodium starch glycolate, croscarmellose, alginic acid, and sodium carboxymethyl cellulose. Suitable lubricants include stearic acid, magnesium stearate, calcium stearate, and talc.

In another aspect, the invention is directed to a parenteral formulation, e.g., in-situ gels, microspheres, nanospheres, nanosuspensions, or lyophilized products to control the release of a compound following subcutaneous and/or intramuscular administration, comprising a compound of the invention, a surfactant and/or a polymeric carrier and/or a solubilising excipient and/or an excipient to control osmolality. Suitable surfactants include polysorbates, polyvinyl alcohol, polyvinyl pyrrolidone and combinations thereof. Suitable polymeric carriers include polyethylene glycol, polymethacrylate, ethylene vinyl acetate copolymer, polyglactin, polyoxyethylene fatty acid esters, poly(lactic-co-glycolic acid), poly(epsilon-caprolactone), poly(p-dioxanone), poly(anhydride esters) and combinations thereof. Suitable solubilising excipients include n-methyl pyrollidone, polyethoxylated castor oil (e.g., CREMOPHOR such as CREMOPHOR EL), polysorbates, Solutol® (Macrogol 15 Hydroxystearate Ph. Eur; Polyoxyl 15 Hydroxystearate USP), ethanol and combinations thereof. Suitable excipients to control osmolality (and in the case of lyophiles, to bulk the lyophilized material) include mannitol, sucrose, glycine, and polyvinyl pyrrolidone.

In-situ gels can be prepared by solubilising a compound of the invention in solvent phase and water-insoluble polymeric carrier(s). The solution is then sterilized, e.g., by gamma irradiation.

Nanosuspensions or micron-sized suspensions can be prepared by combining a compound of the invention, a surfactant, a polymeric carrier and an excipient to control osmolality in aqueous phase, then bead milling or microfluidising the combination in aqueous phase to deliver particles of the compound sized less than 5 μm, e.g., less than 1 μm, or e.g., between 100 nm to less than 5 μm or to less than 1 μm. The nanosuspension is sterilized, e.g., by utilizing terminal heat sterilization or gamma irradiation techniques.

Microspheres and nanospheres can be prepared by various methods known in the art including water/oil/water emulsion methods, solvent/oil/water emulsion methods, oil/water emulsion methods, organic phase separation or melt extrusion/cryomilling techniques which involve inclusion of the compound of the invention and polymer(s) to control drug delivery. The particles are delivered to less than 100 μm for microspheres and between 100 nm to less than 1 μm for nanospheres. The microspheres and nanospheres can go through further processing, including lyophilization, and require sterilization, e.g., through gamma irradiation.

A lyophilized product may suitably include a compound of the invention in a concentration of from 0.01-100 mg/mL, a surfactant, a polymeric carrier, and a solubilizing excipient. General conditions to provide a lyophilized product involve forming a solution or suspension of the product ingredients, reducing the solution or suspension below the glass transition, providing differential pressure to pull off aqueous and/or solvent phase, and slowly increasing temperature to form a lyophilized cake.

In another aspect, the pharmaceutical composition is in liquid bulk or unit dosage form, e.g. for oral, topical or parenteral administration. For example, a compound of the invention, as a pre-drug, may be combined with one more suitable pharmaceutically acceptable excipients to provide a liquid composition in which a BMP1, TLL1 and/or TLL2 inhibitor is revealed prior to administration of the composition to the subject. Such compositions suitably contain, in addition to a compound of the invention, pharmaceutically acceptable excipients selected from but not limited to sodium hydroxide, hydrochloric acid, sodium chloride, mannitol, polyethylene glycol, cyclodextrin, tromethamine (or other suitable buffer) and water.

In some embodiments, a compound of the invention converts to a BMP1, TLL1 and/or TLL2 inhibitor when combined with one or pharmaceutically acceptable excipients. Accordingly, it will be understood that the present invention encompasses a composition, e.g. a pharmaceutical composition, formed by combining a compound of the invention with at least one pharmaceutically acceptable excipient, It will be further understood that the present invention also encompasses methods of treatment, uses, compositions, and combinations analogous to those described herein, comprising administration or use of a composition, e.g., a pharmaceutical composition, formed by combining a compound of the invention with one or more pharmaceutically acceptable excipients.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

It will be understood by the skilled artisan that purification methods (using acidic or basic modifiers) or compound workup procedures (using acidic or basic conditions) may result in formation of a salt of a title compound (for example, hydrobromic acid, formic acid, hydrochloric acid, trifluoroacetic acid, or ammonia salts of a title compound). The present invention is intended to encompass such salts. In the Examples the parent compound is depicted structurally.

In the following experimental descriptions, the following abbreviations may be used:

| Abbreviation | Meaning |
|---|---|
| AcOH | acetic acid |
| aq. | aqueous |
| BBr₃ | boron tribromide |
| BCl₃ | boron trichloride |
| BH₃ | borane |
| Bn | benzyl |
| brine | saturated aqueous sodium chloride |
| BuLi | butyl lithium |
| CDI | carbonyldiimidazole |
| CH₂Cl₂ | methylene chloride |
| COCl₂ | oxalyl chloride |
| DCC | dicyclohexylcarbodiimide |
| DCM | methylene chloride |
| DEAD | Diethyl azodicarboxylate |
| DEAP | diethyl aminopyridine |
| DIAD | diisopropyl azodicarboxylate |
| DIPEA | N,N-diisopropylethylamine |
| DMAP | 4-dimethylaminopyridine |
| DMF | N,N-dimethylformamide |
| DME | dimethoxyethane |
| DMSO | dimethylsulfoxide |
| EDC | 1-[3-(dimethylamino)propyl]-3-ethylcarbodiimide |
| | hydrochloride |
| Et | ethyl |
| Et₃N | triethylamine |
| Et₂O | diethyl ether |
| EtOAc | ethyl acetate |
| EtOH | ethanol |
| Fmoc | fluorenylmethyloxycarbonyl |
| h | hour(s) |
| H₂ | hydrogen |
| H₂O₂ | hydrogen peroxide |
| H₂O | water |
| H₂SO₄ | sulfuric acid |
| HATU | (O-(7-azabenzotriazol-1-yl)-N,N,N′,N′- |
| | tetramethyluronium hexafluorophosphate) |
| HBTU | 2-(1H-benzo[d][1,2,3]triazol-1-yl)-1,1,3,3- |
| | tetramethylisouronium hexafluorophosphate(V) |
| HCl | hydrochloric acid |
| HCO₂H | formic acid |
| HOBt | 1-hydroxybenzotriazole |
| HPLC | high performance liquid chromatography |
| I₂ | Iodine |
| JLR | jacketed lab reactor |
| K₂CO₃ | potassium carbonate |
| KHSO₄ | potassium hydrogen sulfate |
| KOAc | potassium acetate |
| LAH | Lithium aluminum hydride |
| LCMS | liquid chromatography-mass spectroscopy |
| LDA | lithium diisopropyl amide |
| LiOH | lithium hydroxide |
| LHMDS | lithium bis(trimethylsilyl)amide |
| Me | methyl |
| MeOH | methanol |
| MeCN | acetonitrile |
| MgBr₂ | magnesium bromide |
| MgSO₄ | magnesium sulfate |
| min or mins | minute(s) |
| MS | mass spectrum |
| MTBE | Methyl tert-butyl ether |
| qs | Quantum Satis |
| μw | microwave |
| N₂ | nitrogen |
| Na(CN)BH₃ | sodium cyanoborohydride |
| NaCl | sodium chloride |
| Na₂CO₃ | sodium carbonate |
| NaHCO₃ | sodium bicarbonate |
| NaHMDS | sodium bis(trimethylsilyl)amide |
| NaHSO₃ | sodium bisulfite |
| NaH | sodium hydride |
| NaI | sodium iodide |
| NaOH | sodium hydroxide |
| Na₂SO₃ | sodium sulfite |
| Na₂SO₄ | sodium sulfate |
| NH₄Cl | ammonium chloride |
| HCO₂•NH₄ | ammonium formate |
| NH₄OH | ammonium hydroxide |
| NMO | 4-methylmorpholine N-oxide |
| NMP | N-methyl-2-pyrrolidone |
| Pd/C | 10% weight palladium on carbon |
| PdCl₂(dbpf) | 1,1′-bis(di-tert-butylphosphino)ferrocene |
| | dichloropalladium |
| Pd(dppf)Cl₂/ | [1,1′-bis(diphenylphosphino)ferrocene] |
| PdCl₂(dppf) | dichloropalladium(II) |
| Pd(Ph₃)₄ | tetrakis(triphenylphosphine)palladium(0) |
| Pd(OAc)₂ | palladium acetate |
| Pd(OH)₂ | palladium hydroxide |
| Ph | phenyl |
| PIFA | [Bis(trifluoroacetoxy)iodo]benzene |
| PL HCO₃ MP | macroporus polystyrene supported carbonate |
| POCl₃ | phosphoryl chloride |
| PTFE | polytetrafluoroethylene |
| rRt or RT | room temperature |
| sat. | saturated |
| SFC | supercritical fluid chromatography |
| Si | silica |
| SPE | solid phase extraction |
| T3P ® | propylphosphonic anhydride |
| TBAF | tetrabutylammonium fluoride |
| TBAI | tetrabutylammonium iodide |
| TBDMSCl | tert-butyldimethylsilyl chloride |
| TBME | tert-butylmethyl ether |
| TFA | trifluoroacetic acid |
| THF | tetrahydrofuran |
| TiCl₄ | titanium tetrachloride |
| TMS-Br | trimethylsilyl bromide |
| TMS-Cl | trimethylsilyl chloride |
| TMS-OTf | trimethylsilyl triflate |
| tR | retention time |
| UPLC | ultra performance liquid chromatography |

### Intermediate 1: (9H-fluoren-9-yl)methyl (aminomethyl)carbamate, 4-methylbenzenesulphonic Acid Salt

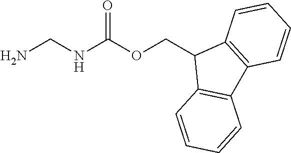 [SMILES: NCNC(=O)OCC1c2ccccc2-c2ccccc21]

### Step 1: (9H-fluoren-9-yl)methyl (2-amino-2-oxoethyl)carbamate

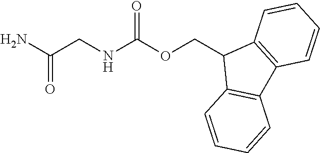 [SMILES: NC(=O)CNC(=O)OCC1c2ccccc2-c2ccccc21]

2-aminoacetamide, hydrochloride (250 g, 2148 mmol) was taken up in DCM (12 L). The reaction was mixed with overhead stirring. DIPEA (826 mL, 4727 mmol) was added slowly and the reaction was stirred at RT for 15 min. (9H-Fluoren-9-yl)methyl carbonochloridate (Fmoc-Cl) (582 g, 2256 mmol) was added in 5 equal portions over 45 min. Stirring was continued for 1 h and diluted with DCM (1.1 L). The reaction was quenched with water (1.33 L) and stirring was continued at RT for 20 min. The product was isolated by filtration and was dried on the funnel 1 h. The cake was then crushed with a mortar and pestle and dried under high vacuum without heat to afford the title compound as a pale yellow solid (77% purity). (1272 g, 100%)

### Step 2: (9H-fluoren-9-yl)methyl (aminomethyl)carbamate, 4-methylbenzenesulphonic Acid Salt

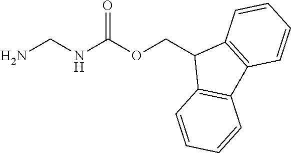 [SMILES: NCNC(=O)OCC1c2ccccc2-c2ccccc21]

A 6 L jacketed laboratory reactor with overhead stirring and Huber temperature control was charged with (9H-fluoren-9-yl)methyl (2-amino-2-oxoethyl)carbamate (40 g, 64.8 mmol) and EtOAc (384 mL). The suspension was stirred and heated to 50° C. (internal temp). Most of the solids dissolved. The warm mixture was washed with 5 wt % aqueous NaHCO₃ (48 mL), charged slowly to control any off-gassing. The mixture was then washed twice with water (48 mL). The reaction was cooled to 20° C., during which the starting material precipitated. To the cooled mixture was added (bis(trifluoroacetoxy)iodo)benzene (PIFA) (41.9 g, 97.2 mmol) as a solid in one portion, followed by water (19.2 mL) The mixture was observed to nearly form a solution within ˜5 mins following the water addition. The reaction was stirred at 20° C. for 90 min. Agitation of the mixture was increased, then a solution of p-toluenesulfonic acid (pTsOH) (13.1 g, 68 mmol) in water (19.2 mL) was added over 30 min. Mixture opacity increased during the charge, beginning when ˜25% had been added. The mixture was a slurry once ˜66% had been added. The slurry was stirred at 15° C. for 30 min, then at 0° C. for 40 min. The solids were isolated via vacuum filtration. The cake was washed with MeCN (48 mL). The solids were dried in a vacuum oven without heat for 12-20 h, then a slow stream of N2 was introduced while the product remained under vacuum for 4 h to afford the title compound as a colorless solid. (21.8 g, 76%).

### Intermediate 2: (R)—N-(aminomethyl)-2-((R)-1-(N-hydroxyformamido)propyl)heptanamide

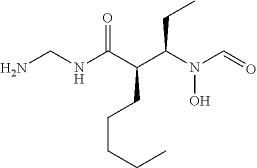 [SMILES: CCCCC[C@@H](C(=O)NCN)[C@@H](CC)N(O)C=O]

### Step 1: (R)-4-benzyl-3-heptanoyloxazolidin-2-one

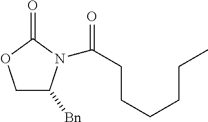 [SMILES: CCCCCCC(=O)N1C(=O)OC[C@H]1Cc1ccccc1]

(R)-4-benzyloxazolidin-2-one (9.95 g, 56.2 mmol) was dissolved in dry THF (100 mL) and the mixture cooled in a dry-ice acetone bath. BuLi (2.7 M in hexanes, 20.80 mL, 56.2 mmol) was added over 5 min under nitrogen resulting in a color change to dark yellow. The color was titrated out by addition of HCl in dioxane, then re-treated with enough BuLi to turn the mixture slightly yellow. Heptanoyl chloride (8.87 mL, 57.3 mmol) was then added. The mixture was stirred for ˜30 min and then additional heptanoyl chloride (3 mL) was added. The reaction was then quenched by slow addition of water (10 mL). Solid formation was noted, additional water was added to obtain a solution. EtOAc (300 mL) was added and the layers separated. The organic was washed with sat. aq. sodium carbonate then dried over sodium sulfate, filtered and concentrated. The residue was dissolved in heptane, and then concentrated to a thick oil. The residue was dissolved in heptane (100 mL) and the mixture cooled in an acetone/dry ice bath with stirring. The resultant precipitate was collected by filtration and dried under vacuum overnight to give the title compound as a white solid. (15.1 g, 93% yield).

### Step 2: (R)-4-benzyl-3-((R)-2-((S)-1-hydroxypropyl)heptanoyl)oxazolidin-2-one

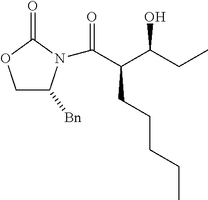 [SMILES: CCCCC[C@@H](C(=O)N1C(=O)OC[C@H]1Cc1ccccc1)[C@@H](O)CC]

To a solution of (R)-4-benzyl-3-heptanoyloxazolidin-2-one (15 g, 51.8 mmol) in DCM (300 mL) under N₂ in a ice-acetone bath was added TiCl₄ (6.00 mL, 54.4 mmol). DIPEA (9.96 mL, 57.0 mmol) was then slowly added followed by NMP (9.98 mL, 104 mmol) and the mixture stirred for 15 min. Propionaldehyde (7 mL, 95 mmol) was then added and the reaction stirred for 1.5 h. The reaction was then quenched by addition of a solution of AcOH in DCM (15 mL of a 50:50 mixture by volume). Saturated aqueous Rochelles salt was added followed by aq. HCl (50% v/v) to dissolve any solids. The layers were then separated and the aqueous extracted with additional DCM. The combined organics were treated with aq. NaHSO₃ for 30 min and the layers allowed to settle in a separation funnel overnight. The organic phase was then separated and filtered through a plug of silica (˜3 cm). The filtrates were combined, concentrated and dried under vacuum to give the title compound (19 g, 87% yield) which was used without further purification.

### Step 3: (R)—N-(benzyloxy)-2-((S)-1-hydroxypropyl)heptanamide

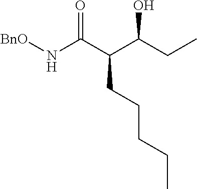 [SMILES: CCCCC[C@@H](C(=O)NOCc1ccccc1)[C@@H](O)CC]

THF was boiled out of a 2 L JLR and the reactor purged with N₂ whilst cooling to rt. O-benzylhydroxylamine, hydrochloride (15.96 g, 100 mmol) was added and the vessel purged with N₂. THF (800 mL) was then added and the mixture cooled to 0° C. Trimethylaluminum (50 mL, 2 M in toluene, 100 mmol) was then added slowly. The white slurry was stirred for 15 min to obtain a clear solution. A solution of (R)-4-benzyl-3-((R)-2-((S)-1-hydroxypropyl)heptanoyl)oxazolidin-2-one (18 g, 51.8 mmol) in THF (200 mL) was then added over 5 min via cannula and the mixture stirred for 1.5 h at 0° C. The reaction mixture was warmed to 5° C. Separately, O-benzylhydroxylamine, hydrochloride (5 g, 31 mmol) was dissolved in THF (100 mL) and treated with trimethylaluminum (17 mL, 2 M in toluene, 34 mmol) at 0° C. The mixture was stirred until a solution was obtained and then added to the primary reaction via cannula. The reaction was then quenched by the addition of sat. aq. KHSO₄. A HCl solution (500 mL water, 500 mL conc HCl) was added and the layers separated. The organics were reduced in volume and re-combined with the aqueous. The volatiles were removed via rotovap and a white precipitate formed. The solids were collected by filtration and washed with 10% HCl followed by water. The filter cake was then washed with toluene (2×100 mL) and air dried to give the title compound (10.65 g, 70% yield). MS (m/z) 294.3 (M+H)⁺

### Step 4: (3R,4R)-1-(benzyloxy)-4-ethyl-3-pentylazetidin-2-one

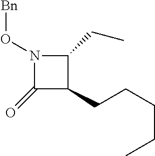 [SMILES: CCCCC[C@H]1C(=O)N(OCc2ccccc2)[C@@H]1CC]

R)—N-(benzyloxy)-2-((S)-1-hydroxypropyl)heptanamide (4.61 g, 15.7 mmol) was dissolved in pyridine (14 mL) and cooled in an ice bath. Methanesulfonyl chloride (2.45 mL, 31.4 mmol) was then added drop wise maintaining the internal temperature below 10° C. The reaction was then stirred for 2 h. The reaction was diluted by the addition of TBME (23 mL). The diluted reaction solution was cooled in an ice bath and treated with 1 M HCl (46 mL). The layers were separated and the organic was washed with 1 M HCl (23 mL), sat. aq. NaHCO₃ (9 mL) and brine (9 mL) and then concentrated to minimum volume. The crude was dissolved in acetone (46 mL), treated with K₂CO₃ (6.51 g, 47.1 mmol), and the resulting reaction heated at 50° C. for 1 h. The reaction was then cooled to RT and filtered. The flask and filter cake were rinsed with acetone (2×23 mL). The filtrate was concentrated give the title compound (4.26 g, 98% yield).

### Step 5: (R)-2-((R)-1-((benzyloxy)amino)propyl)heptanoic Acid

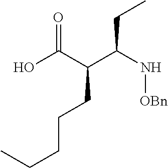 [SMILES: CCCCC[C@@H](C(=O)O)[C@@H](CC)NOCc1ccccc1]

(3R,4R)-1-(benzyloxy)-4-ethyl-3-pentylazetidin-2-one (356 g, 1.27 mol) was dissolved in 2-methyltetrahydrofuran (3560 mL). Tetrabutylammonium hydroxide (40% aqueous solution, 1245 mL, 1.90 mol) was added. The reaction was heated to 50° C. for 2 h and then cooled to rt. The reaction was diluted with water (1780 mL) and acidified with 6 M HCl (338 mL) to pH 3-4. The phases were separated and the organic phase was concentrated down to 5 volumes (1780 mL) and was used without further purification.

### Step 6: (R)-2-((R)-1-(N-(benzyloxy)formamido)propyl)heptanoic Acid

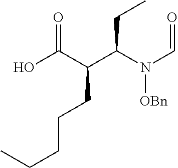 [SMILES: CCCCC[C@@H](C(=O)O)[C@@H](CC)N(C=O)OCc1ccccc1]

Carbonyldiimidazole (822 g, 5.07 mol) was suspended in 2-methyltetrahydrofuran (5340 mL) and cooled to 0° C. Formic acid (88%, 276 mL, 6.33 mol) was added drop wise via addition funnel. The reaction was stirred at 5° C. for 10 min and then warmed to rt for an additional 30 min. The reaction was cooled back to 5° C. and (R)-2-((R)-1-((benzyloxy)amino)propyl)heptanoic acid in 2-methyltetrahydrofuran (1780 mL solution from previous step) was added. The reaction was warmed to RT and stirred for 40 min. In a separate vessel, carbonyldiimidazole (279 g, 1.72 mol) was suspended in 2-methyltetrahydrofuran (1500 mL) and cooled to 0° C. Formic acid, (88%, 93.8 mL, 2.16 mol) was added drop wise via addition funnel and stirred at 5° C. for 10 min and then warmed to rt for an additional 30 min. This mixture was then added drop wise via addition funnel to the original reaction at 5° C. The reaction was warmed to RT and stirred for 60 min. The reaction was then cooled to 10° C. and quenched by addition of NaOH (4 M, 2122 mL) to pH 9. The phases were separated and the organic phase was washed with a 1:1 mixture (v/v) of 6 M HCl and saturated aqueous brine (4561 mL). The phases were separated and the organic phase was concentrated to 3.5 volumes (1246 mL) to give the title compound as a 30% by weight solution in 2-methyltetrahydrofuran (1.15 kg, equates to 346 g of crude title product).

Steps 5 and 6 were repeated with (3R,4R)-1-(benzyloxy)-4-ethyl-3-pentylazetidin-2-one (365 g) scale to provide a second batch (R)-2-((R)-1-(N-(benzyloxy)formamido)propyl)heptanoic acid as a 36% by weight solution in 2-methyltetrahydrofuran (1.09 kg, equates to 363 g of crude title product). This process (steps 5 and 6) was repeated again on 20 g scale to provide (R)-2-((R)-1-(N-(benzyloxy)formamido)propyl)heptanoic acid as a 30% by weight solution in 2-methyltetrahydrofuran (66 g, equates to 19.8 g of crude title product).

The 30% by weight solution of crude title compound in 2-methyltetrahydrofuran (1.15 kg, 346 g crude) was concentrated, azeotroped three times with hexanes and then diluted with hexanes (2500 mL). The solution was seeded with crystals obtained from a previous SFC purification. Nitrogen was then passed over the solution with stirring overnight. The resulting crystalline material was broken up, diluted with hexanes and stirred at RT for 30 min then filtered to give the title compound as a light yellow crystalline solid (275 g). The 36% (1.09 kg, 363 g crude) and 30% (66.04 g, 19.8 g crude) by weight solutions of crude title compound were concentrated, azeotroped three times with MeOH and combined with the filtrate from the initial 30% by weight batch. The residue was diluted with MeOH to a concentration of 200 mg/mL and purified by SFC (Thar SFC-70, DEAP column, 5 μM, 30×250 mm, i.d., eluting with 35% isocratic MeOH co-solvent, 60 g/min, 7 minute run) to give the title compound as a yellow oil. The oil was diluted with hexanes (2500 mL) and the solution seeded with crystals obtained from previous isolates. Nitrogen was passed over the solution with stirring overnight. The resulting crystalline material was broken up, diluted with hexanes, and stirred at RT for 30 min then filtered to give the title compound as a light yellow crystalline solid (360 g). MS (m/z) 322.0 (M+H)⁺

### Step 7: (9H-fluoren-9-yl)methyl (((R)-2-((R)-1-(N-(benzyloxy)formamido)propyl)heptanamido)methyl)carbamate

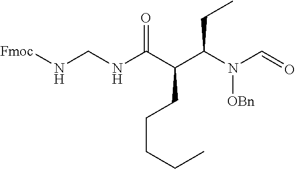

To a solution containing (R)-2-((R)-1-(N-(benzyloxy)formamido)propyl)heptanoic acid (47 g, 146 mmol) in THF (937 mL) at 0° C. with an ice bath was added HATU (55.6 g, 146 mmol) and then DIPEA (77 mL, 439 mmol). The reaction was stirred for 10 min at 0° C. (9H-fluoren-9-yl)methyl (aminomethyl)carbamate, 4-Methylbenzenesulphonic acid salt (64.4 g, 146 mmol) was added portion wise and the reaction was stirred 10 min at 0° C., then at rt for 2 h. CH₃CN (500 mL) was added and reaction stirred overnight. The reaction was filtered through celite, and the filtrate was concentrated and partitioned between EtOAc and water. The aqueous phase was extracted with EtOAc 3×. The combined organic phases were dried with MgSO₄, filtered and concentrated to afford the title product as a yellow oil. (84 g, 100% yield). MS (m/z) 572.4 (M+H)⁺

### Step 8: (R)—N-(aminomethyl)-2-((R)-1-(N-(benzyloxy)formamido)propyl)heptanamide

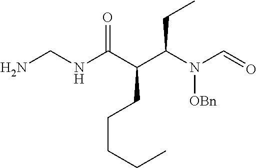 [SMILES: CCCCC[C@@H](C(=O)NCN)[C@@H](CC)N(C=O)OCc1ccccc1]

To a solution containing (9H-fluoren-9-yl)methyl (((R)-2-((R)-1-(N-(benzyloxy)formamido)propyl)heptanamido)methyl)carbamate (84 g, 147 mmol) in THF (466 mL) was added dihexylamine (317 mL, 1469 mmol) and the reaction mixture was stirred at rt for 18 h. The reaction mixture was concentrated and the resulting residue was triturated with 500 mL hexanes. The hexanes layer was decanted and the process repeated 5 times until the oil becomes very thick/viscous and sticky light yellow colored. The crude oil was then taken up in 50:50 DCM/MeOH and concentrated onto Si. Purification on Si (750 g) with 0-10% MeOH in DCM as eluant afforded the title compound as a yellow oil (35.11 g, 68.4% yield). MS (m/z) 350.0 (M+H)⁺

### Step 9: (R)—N-(aminomethyl)-2-((R)-1-(N-hydroxyformamido)propyl)heptanamide

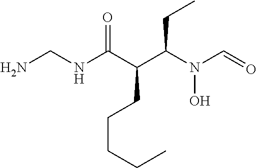 [SMILES: CCCCC[C@@H](C(=O)NCN)[C@@H](CC)N(O)C=O]

To a 1000 mL RBF was added Pd—C (4.89 g, 4.59 mmol), followed by addition of DCM (17.60 mL) under nitrogen. A mixture of (R)—N-(aminomethyl)-2-((R)-1-(N-(benzyloxy)formamido)propyl)heptanamide (32.11 g, 92 mmol) in EtOH (616 mL) was slowly added under nitrogen. The reaction was then sealed and purged with nitrogen. A hydrogen balloon was attached and the reaction was stirred at RT overnight. The reaction was filtered through a large thick plug of celite, washed with EtOAc, and concentrated to get a dark brown oil. The oil was triturated with 1000 mL hexanes. An off-white solid precipitated, which was filtered with washing in excess hexane, and then air dried to obtain the title compound as a colorless solid (18.22 g, 76% yield). MS (m/z) 260.2 (M+H)⁺

### Intermediate 3: (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate

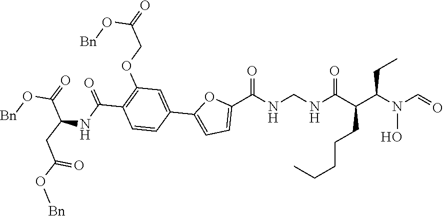 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(O)C=O]

### Step 1: (S)-dibenzyl 2-(4-bromo-2-hydroxybenzamido)succinate

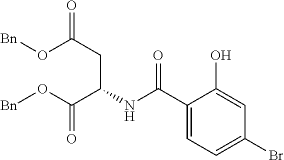 [SMILES: O=C(C[C@H](NC(=O)c1ccc(Br)cc1O)C(=O)OCc1ccccc1)OCc1ccccc1]

To a solution containing (S)-dibenzyl 2-aminosuccinate, 4-Methylbenzenesulfinate salt (56.7 g, 121 mmol) and 4-bromo-2-hydroxybenzoic acid (25 g, 115 mmol) and HATU (52.6 g, 138 mmol) in DMF (150 mL) was added DIPEA (40.2 mL, 230 mmol). The reaction mixture was stirred for 18 h at RT. Reaction mixture was diluted with NH₄Cl aq. (450 mL), extracted with EtOAc (2×250 mL). The organic layer was washed with NH₄Cl aq. (200 mL), and brine (200 mL), dried over MgSO₄, filtered and concentrated onto Si. Purification on Si (220 g) with (0-40% EtOAc/hexanes) as eluant afforded the title compound as a colorless glass (37.86 g, 64.1% yield). MS (m/z) 512.4 (M+H)⁺

### Step 2: (S)-dibenzyl 2-(2-(2-(benzyloxy)-2-oxoethoxy)-4-bromobenzamido)succinate

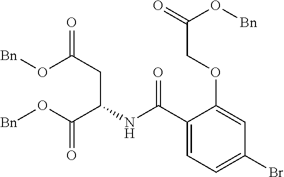 [SMILES: O=C(COc1cc(Br)ccc1C(=O)N[C@@H](CC(=O)OCc1ccccc1)C(=O)OCc1ccccc1)OCc1ccccc1]

To a solution containing (S)-dibenzyl 2-(4-bromo-2-hydroxybenzamido)succinate (37.86 g, 73.9 mmol) in Acetone (200 mL) was added benzyl 2-bromoacetate (12.88 mL, 81 mmol) and potassium carbonate (20.43 g, 148 mmol). The reaction mixture was stirred for 5 h and diluted with water (500 mL) and extracted with EtOAc (2×300 mL). Organic phase was dried over MgSO₄, filtered and concentrated onto Si. Purification on Si (330 g) with (0-40% EtOAc/hexanes) as eluant afforded the title compound as a pale yellow glass (42.26 g, 87% yield). MS (m/z) 660.5 (M+H)⁺

### Step 3: (S)-dibenzyl 2-(2-(2-(benzyloxy)-2-oxoethoxy)-4-(4,4,5,5-tetramethyl-1,3,2-dioxaborolan-2-yl)benzamido)succinate

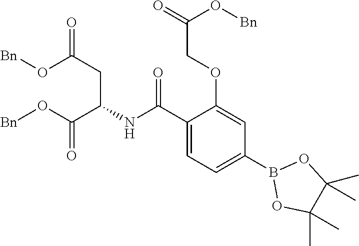 [SMILES: CC1(C)OB(c2ccc(C(=O)N[C@@H](CC(=O)OCc3ccccc3)C(=O)OCc3ccccc3)c(OCC(=O)OCc3ccccc3)c2)OC1(C)C]

(S)-dibenzyl 2-(2-(2-(benzyloxy)-2-oxoethoxy)-4-bromobenzamido)succinate (13.18 g, 19.95 mmol) in 1,4-Dioxane (110 mL) was degassed for 5 min with N₂. At this time, 4,4,4′,4′,5,5,5′,5′-octamethyl-2,2′-bi(1,3,2-dioxaborolane) (5.07 g, 19.95 mmol), potassium acetate (5.88 g, 59.9 mmol), and PdCl₂(dppf)-CH₂Cl₂ (1.467 g, 1.796 mmol) were added and degassed for 5 min with N₂ and then heated to 90° C. for 18 h under N₂. The reaction mixture was cooled, diluted with EtOAc (400 mL) and brine (400 mL) and filtered through celite. The celite was washed with EtOAc (200 mL). The layers were separated and the organics dried over MgSO₄, filtered and concentrated onto SiO₂. Purification on Si (220 g) with (0-40% EtOAc/hexanes) as eluant afforded the title compound as a tan oil (10.05 g, 71.2% yield). MS (m/z) 708.4 (M+H)⁺

### Step 4: (S)-dibenzyl 2-(2-(2-(benzyloxy)-2-oxoethoxy)-4-(5-(tert-butoxycarbonyl)furan-2-yl)benzamido)succinate

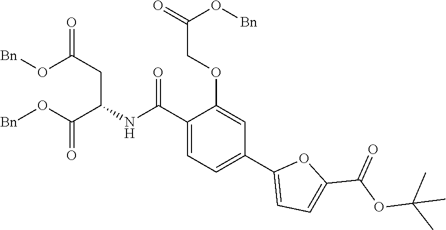 [SMILES: CC(C)(C)OC(=O)c1ccc(-c2ccc(C(=O)N[C@@H](CC(=O)OCc3ccccc3)C(=O)OCc3ccccc3)c(OCC(=O)OCc3ccccc3)c2)o1]

(S)-dibenzyl 2-(2-(2-(benzyloxy)-2-oxoethoxy)-4-(4,4,5,5-tetramethyl-1,3,2-dioxaborolan-2-yl)benzamido)succinate (9.8 g, 13.85 mmol) in 1,4-Dioxane (100 mL) was degassed for 10 min with N₂. At this time, tert-butyl 5-bromofuran-2-carboxylate (3.76 g, 15.24 mmol), sodium carbonate (1M aq. solution, 41.6 mL, 41.6 mmol), and PdCl₂(dppf)-CH₂Cl₂ (0.622 g, 0.762 mmol) were added and degassed for 5 min with N₂ and then heated to 50° C. for 30 min under N₂. The reaction mixture was cooled to ambient temperature and diluted with EtOAc (300 mL) and washed with water (300 mL). The aqueous phase was back extracted with EtOAc (100 mL). The organic phase was combined, dried over MgSO₄, filtered and concentrated onto Si. Purification on Si (330 g) with (0-40% EtOAc/hexanes) as eluant afforded the title compound as a pale brown oil (15.67 g, 78% yield). MS (m/z) 747.9 (M+H)⁺

### Step 5: (S)-5-(3-(2-(benzyloxy)-2-oxoethoxy)-4-((1,4-bis(benzyloxy)-1,4-dioxobutan-2-yl)carbamoyl)phenyl)furan-2-carboxylic Acid

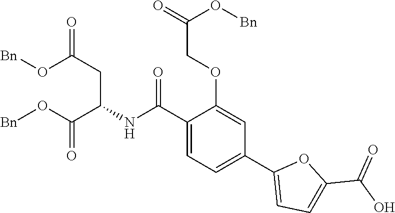 [SMILES: O=C(COc1cc(-c2ccc(C(=O)O)o2)ccc1C(=O)N[C@@H](CC(=O)OCc1ccccc1)C(=O)OCc1ccccc1)OCc1ccccc1]

To a solution containing (S)-dibenzyl 2-(2-(2-(benzyloxy)-2-oxoethoxy)-4-(5-(tert-butoxycarbonyl)furan-2-yl)benzamido)succinate (15.67 g, 20.96 mmol) in DCM (200 mL) was added TFA (32.3 mL, 419 mmol). The reaction was stirred at room temp for 18 h. The reaction mixture was concentrated, azeotroped 2×DCM (100 mL), 2× toluene (100 mL), and 1×CH₃CN (100 mL) to give the title compound as brown foam (14.49 g, 100% yield). MS (m/z) 691.9 (M+H)⁺

### Step 6: (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate

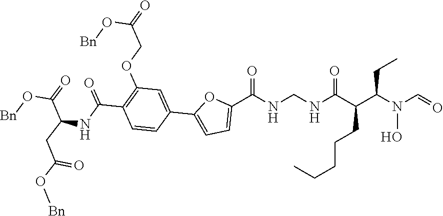 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(O)C=O]

To a solution containing (S)-5-(3-(2-(benzyloxy)-2-oxoethoxy)-4-((1,4-bis(benzyloxy)-1,4-dioxobutan-2-yl)carbamoyl)phenyl)furan-2-carboxylic acid (0.76 g, 1.099 mmol) in DMF (5 mL) was added DIPEA (0.576 mL, 3.30 mmol) followed by isopropyl carbonochloridate (1.209 mL, 1.209 mmol). The reaction mixture was stirred for 15 min. The activated ester was added drop wise to a solution containing (R)—N-(aminomethyl)-2-((R)-1-(N-hydroxyformamido)propyl)heptanamide (0.299 g, 1.154 mmol) in DMF (5 mL) at 0° C. The reaction was stirred for 1 h at 0° C. and then warmed to RT for 2 h. The reaction was poured into NH₄Cl aq., extracted with EtOAc, and washed with brine. The organics were dried over MgSO₄, filtered, and concentrated onto SiO₂. Purification on Si (40 g) with (0-100% EtOAc/hexanes) as eluant afforded the title compound as a brown foam (0.51 g, 50% yield). MS (m/z) 933.4 (M+H)⁺

### Intermediate 4: (S)-dibenzyl 2-(2-ethoxy-4-(5-((((R)-2-((R)-1-(N-hydroxyformamido)propyl)heptanamido)methyl)carbamoyl)furan-2-yl)benzamido)succinate

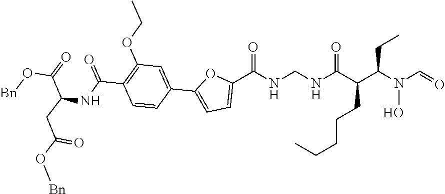 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(O)C=O]

### Step 1: Ethyl 4-bromo-2-ethoxybenzoate

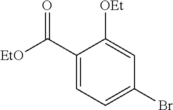 [SMILES: CCOC(=O)c1ccc(Br)cc1OCC]

Ethyl iodide (74.5 mL, 922 mmol) was added drop wise to a solution containing 4-bromo-2-hydroxybenzoic acid (50.0 g, 230 mmol) and potassium carbonate (159 g, 1152 mmol) in DMF (200 mL). The mixture was stirred at RT for 18 h. Water and EtOAc were added and the organics were washed with brine (3×). The combined washes were extracted with EtOAc, and the extracts were combined with the washed EtOAc layer. The combined organic phase was dried over MgSO4, filtered, and concentrated. Purification on Si with (0-50% EtOAc/hexanes) as eluant afforded the title compound as a yellow solid (54.7 g, 87% yield). MS (m/z) 272.9 (M+H)⁺

### Step 2: 4-bromo-2-ethoxybenzoic Acid

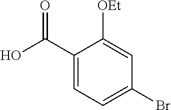 [SMILES: CCOc1cc(Br)ccc1C(=O)O]

To a solution containing ethyl 4-bromo-2-ethoxybenzoate (54.7 g, 200 mmol) in THF (668 mL) was added 6M NaOH (100 mL, 601 mmol). The resulting mixture was stirred for 2 h at RT and was then concentrated to an aqueous slurry. The concentrate was diluted with more water, and 6M HCl (100 mL, 601 mmol) was added. The mixture was stirred for 5 min and was then filtered washing with water to afford the title compound as a colorless solid. (49.3 g, 99% yield). MS (m/z) 244.8 (M+H)⁺

### Step 3: (S)-dibenzyl 2-(4-bromo-2-ethoxybenzamido)succinate

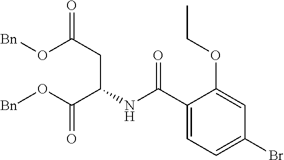 [SMILES: CCOc1cc(Br)ccc1C(=O)N[C@@H](CC(=O)OCc1ccccc1)C(=O)OCc1ccccc1]

To a solution containing 4-bromo-2-ethoxybenzoic acid (49.27 g, 201 mmol), (S)-dibenzyl 2-aminosuccinate 4-methylbenzenesulfonate (117 g, 241 mmol), and HATU (92 g, 241 mmol) in DMF (402 mL) was added DIPEA (105 mL, 603 mmol). The resulting mixture was stirred at RT for 1 h. EtOAc (1 L) and water (1 L) were added. The organics were separated and washed with brine (2×500 mL). The combined washes were extracted with EtOAc (2×500 mL). The combined organics were dried over MgSO₄, filtered, and concentrated. Purification on Si (0-50% EtOAc/Hexanes) afforded the title compound as a yellow gum. (110.4 g, 100% yield). MS (m/z) 540.2 (M+H)⁺

### Step 4: (S)-dibenzyl 2-(2-ethoxy-4-(4,4,5,5-tetramethyl-1,3,2-dioxaborolan-2-yl)benzamido)succinate

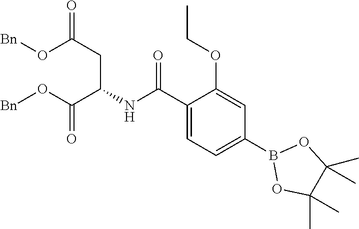 [SMILES: CCOc1cc(B2OC(C)(C)C(C)(C)O2)ccc1C(=O)N[C@@H](CC(=O)OCc1ccccc1)C(=O)OCc1ccccc1]

(S)-dibenzyl 2-(4-bromo-2-ethoxybenzamido)succinate (19.06 g, 35.3 mmol), 4,4,4′,4′,5,5,5′,5′-octamethyl-2,2′-bi(1,3,2-dioxaborolane) (10.75 g, 42.3 mmol), potassium acetate (10.38 g, 106 mmol), and PdCl₂(dppf)-CH₂Cl₂ (2.58 g, 3.53 mmol) in 1,4-Dioxane (118 mL) was degassed for 5 min with N₂. The reaction was heated to 90° C. for 12 h under N₂. Water was added to the cooled reaction, and the organics were extracted with EtOAc (3×). The combined organics were washed with brine (3×), dried over MgSO₄, filtered, and concentrated. Purification on Si(0-75% EtOAc/Hexanes) afforded the title compound as a thick orange oil (17.13 g, 83% yield). MS (m/z) 588.3 (M+H)⁺

### Step 5: (S)-5-(4-((1,4-bis(benzyloxy)-1,4-dioxobutan-2-yl)carbamoyl)-3-ethoxyphenyl)furan-2-carboxylic Acid

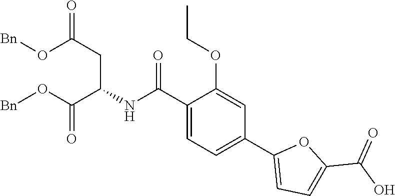 [SMILES: CCOc1cc(-c2ccc(C(=O)O)o2)ccc1C(=O)N[C@@H](CC(=O)OCc1ccccc1)C(=O)OCc1ccccc1]

N₂ was bubbled through a solution of (S)-dibenzyl 2-(2-ethoxy-4-(4,4,5,5-tetramethyl-1,3,2-dioxaborolan-2-yl)benzamido)succinate (17.13 g, 29.2 mmol) in 1,4-Dioxane (194 mL) for 10 min. 5-bromofuran-2-carboxylic acid (6.68 g, 35.0 mmol) and PdCl₂(dppf)-CH₂Cl₂ (1.280 g, 1.57 mmol) were added followed by addition of a 1M Na₂CO₃ aq. solution (87 mL, 87 mmol). The resulting mixture was heated to 50° C. under N₂ for 2 h. The dioxane was evaporated, and EtOAc (500 mL) was added. The aqueous phase was extracted with EtOAc (3×). The combined extracts were washed with brine (2×), dried over MgSO₄, filtered, and concentrated. Purification on Si (0-10% MeOH/EtOAc w/1% AcOH) afforded title compound (5.95 g, 26.8% yield). MS (m/z) 572.2 (M+H)⁺

### Step 6: (S)-dibenzyl 2-(2-ethoxy-4-(5-((((R)-2-((R)-1-(N-hydroxyformamido)propyl)heptanamido)methyl)carbamoyl)furan-2-yl)benzamido)succinate

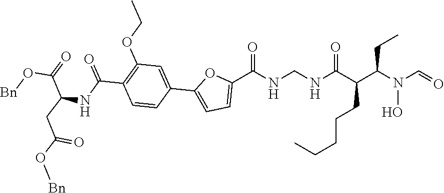 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(O)C=O]

HATU (4.71 g, 12.39 mmol) was added to a stirring mixture of (S)-5-(4-((1,4-bis(benzyloxy)-1,4-dioxobutan-2-yl)carbamoyl)-3-ethoxyphenyl)furan-2-carboxylic acid (5.90 g, 10.32 mmol) and DIPEA (5.27 mL, 31.0 mmol) in CH₃CN (68.8 mL). The resulting mixture was stirred at rt for 15 min. A solution of (R)—N-(aminomethyl)-2-((R)-1-(N-hydroxyformamido)propyl)heptanamide (2.94 g, 11.35 mmol) in DMF (20 mL) was added, and the mixture was stirred for 1 h. Water was added, and the organics were extracted using EtOAc (3×). The combined organics were washed with sat. aq. NaHCO₃, brine (2×), dried over MgSO₄, filtered, and concentrated. Purification on Si (0-10% MeOH/EtOAc) afforded the title compound as a thick brown oil (8.43 g, 90% yield). MS (m/z) 813.4 (M+H)⁺

### Intermediate 5: Dibenzyl (3-ethoxy-5-(5-((((R)-2-((R)-1-(N-hydroxyformamido)propyl)heptanamido)methyl)carbamoyl)furan-2-yl)phenyl)phosphonate

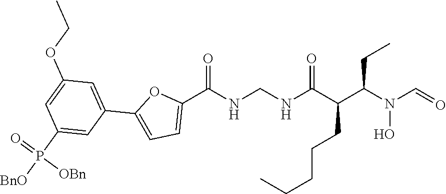 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(O)C=O]

### Step 1: 1-bromo-3-ethoxy-5-iodobenzene

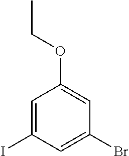

To a solution containing 3-bromo-5-iodophenol (15 g, 50.2 mmol) in CH₃CN (201 mL) was added K₂CO₃ (34.7 g, 251 mmol) followed by iodoethane (4.46 mL, 55.2 mmol). The white suspension was heated to reflux at 80° C. overnight. The reaction was filtered and washed with CH₃CN. The filtrate was concentrated and stirred in hexanes, and filtered washing with excess hexanes. The filtrate was concentrated to obtain the title compound as a yellow oil (16.1 g, 98% yield).

### Step 2: Dimethyl (3-bromo-5-ethoxyphenyl)phosphonate

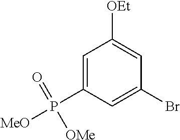 [SMILES: CCOc1cc(Br)cc(P(=O)(OC)OC)c1]

To a solution containing 1-bromo-3-ethoxy-5-iodobenzene (16.1 g, 49.2 mmol) in 1,4-Dioxane (141 mL) was added trimethyl phosphite (14.55 mL, 123 mmol) followed by Pd(OAc)₂ (1.382 g, 6.16 mmol). The reaction was refluxed at 110° C. overnight. The reaction was then cooled, diluted with hexanes and water. The layers were separated, and the aqueous layer was extracted with EtOAc (×3). EtOAc and hexanes layers were combined, washed with brine, dried over MgSO₄, filtered and concentrated. Purification on Si (0-5% MeOH/DCM) afforded the title compound as a yellow brown oil (9.85 g, 65% yield). MS (m/z) 309.0 (M+H)⁺

### Step 3: (3-bromo-5-ethoxyphenyl)phosphonic acid

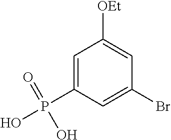 [SMILES: CCOc1cc(Br)cc(P(=O)(O)O)c1]

To a solution containing dimethyl (3-bromo-5-ethoxyphenyl)phosphonate (9.85 g, 31.9 mmol) in DCM (106 mL) was added bromotrimethylsilane (8.27 mL, 63.7 mmol). The reaction was stirred at rt for 2 h. bromotrimethylsilane (4.13 mL, 31.9 mmol) was again added and reaction stirred overnight. The reaction was concentrated, dissolved in 200 mL MeOH, and concentrated. This process was repeated 4 times to afford the title compound as a brown oil (10.8 g, ˜100%) MS (m/z) 280.9 (M+H)⁺

### Step 4: Dibenzyl (3-bromo-5-ethoxyphenyl)phosphonate

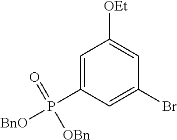 [SMILES: CCOc1cc(Br)cc(P(=O)(OCc2ccccc2)OCc2ccccc2)c1]

To a solution containing (3-bromo-5-ethoxyphenyl)phosphonic acid (6.26 g, 22.3 mmol) in anhydrous DCM (100 mL) was added DMF (26 μL, 0.13 mmol). The reaction mixture was heated to reflux for 25 min followed by addition of oxalyl chloride (5 mL, 57.2 mmol). Evolution of gas was observed. The reaction mixture turned clear brown and was heated for an additional 1 h. The reaction mixture was then concentrated and taken up in anhydrous DCM (100 mL). The solution was cooled to 0° C. Benzyl alcohol was added (7.4 mL, 71.3 mmol) followed by dropwise addition of Et₃N (9.9 mL, 71.3 mmol). (Evolution of gas observed). The reaction mixture was allowed to cool to rt and stir for 12 h. The reaction mixture was washed with water (2×100 mL), dried over Na₂SO₄ and concentrated. Purification on Si (0-100% EtOAc/Hexanes) afforded the title compound as a clear oil (10.2 g, 99% yield). MS (m/z) 461.2 (M+H)⁺

### Step 5 dibenzyl (3-ethoxy-5-(4,4,5,5-tetramethyl-1,3,2-dioxaborolan-2-yl)phenyl)phosphonate

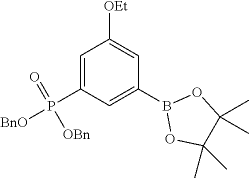 [SMILES: CCOc1cc(B2OC(C)(C)C(C)(C)O2)cc(P(=O)(OCc2ccccc2)OCc2ccccc2)c1]

To a solution containing dibenzyl (3-(bromo)-5-ethoxyphenyl)phosphonate (10.2 g, 22.1 mmol), bis(pinacolato)diboron (6.18 g, 24.3 mmol), and potassium acetate (8.68 g, 88 mmol) in 1,4-Dioxane (110 mL) was added PdCl₂(dppf)-CH₂Cl₂ (0.9 g, 1.1 mmol). The reaction mixture was heated at 105° C. for 12 h and then cooled to rt. The reaction was diluted with water and EtOAc and the layers were separated. The organic phase was concentrated to give a dark residue. Purification on Si (0-100% EtOAc/Hexanes) afforded the title compound as a clear oil (12.4 g, 80%). MS (m/z) 509.3 (M+H)⁺

### Step 6: Tert-butyl 5-(3-(bis(benzyloxy)phosphoryl)-5-ethoxyphenyl)furan-2-carboxylate

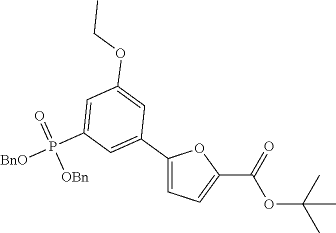 [SMILES: CCOc1cc(-c2ccc(C(=O)OC(C)(C)C)o2)cc(P(=O)(OCc2ccccc2)OCc2ccccc2)c1]

A solution of tert-butyl 5-bromofuran-2-carboxylate (1.4 g, 5.67 mmol) in dioxane/water (21:7 mL) was purged under N₂ for 15 min. PdCl₂(dppf)-CH₂Cl₂ (0.347 g, 0.425 mmol), dibenzyl (3-ethoxy-5-(4,4,5,5-tetramethyl-1,3,2-dioxaborolan-2-yl)phenyl)phosphonate (2.88 g, 5.67 mmol) and sodium carbonate 2M (8.5 mL, 17 mmol) was added and the reaction mixture was stirred under N₂ at 50° C. for 1 h. The reaction mixture was then poured into water and extracted with EtOAc. The combined organic extracts were washed with brine dried over Na₂SO₄ and concentrated. Purification on Si (0-70% EtOAc/Hexanes) afforded the title compound as a clear oil (1.52 g, 50% yield). MS (m/z) 549.2 (M+H)⁺

### Step 7: 5-(3-(bis(benzyloxy)phosphoryl)-5-ethoxyphenyl)furan-2-carboxylic Acid

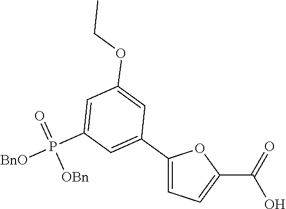 [SMILES: CCOc1cc(-c2ccc(C(=O)O)o2)cc(P(=O)(OCc2ccccc2)OCc2ccccc2)c1]

To a solution containing tert-butyl 5-(3-(bis(benzyloxy)phosphoryl)-5-ethoxyphenyl)furan-2-carboxylate (1.52 g, 2.77 mmol) in DCM (50 mL) was added TFA (4 mL, 52 mmol). The reaction mixture was stirred for 4 h. The reaction was concentrated to dryness to give the title compound as a brown oil (1.33 g, 97% yield). MS (m/z) 492.3 (M+H)⁺

### Step 8: Dibenzyl (3-ethoxy-5-(5-((((R)-2-((R)-1-(N-hydroxyformamido)propyl)heptanamido)methyl)carbamoyl)furan-2-yl)phenyl)phosphonate

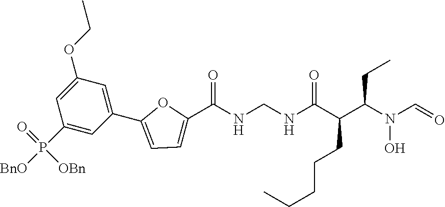 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(O)C=O]

To a solution containing 5-(3-(bis(benzyloxy)phosphoryl)-5-ethoxyphenyl)furan-2-carboxylic acid (1.13 g, 2.3 mmol) in CH₃CN (50 mL) was added Et₃N (0.8 mL, 5.7 mmol) and HATU (0.96 g, 2.52 mmol). The reaction was stirred for 15 min. (R)—N-(aminomethyl)-2-((R)-1-(N-hydroxyformamido)propyl)heptanamide (0.62 g, 2.4 mmol) in DMF (5 mL) was cooled to 0° C. and added to the acid solution. The reaction was stirred for 2 h at which time the mixture was poured into water and extracted into EtOAc. The organic phase was washed with brine, dried over Na₂SO₄, filtered and concentrated. Purification on Si (0-10% MeOH/EtOAc) afforded the title compound as a brown oil (1.2 g, 70% yield). MS (m/z) 734.4 (M+H)⁺

### Intermediate 6: (S)-dibenzyl 2-(4-(5-((((R)-2-((R)-1-(N-(benzoyloxy)formamido)propyl)heptanamido)methyl)carbamoyl)furan-2-yl)-2-ethoxybenzamido)succinate

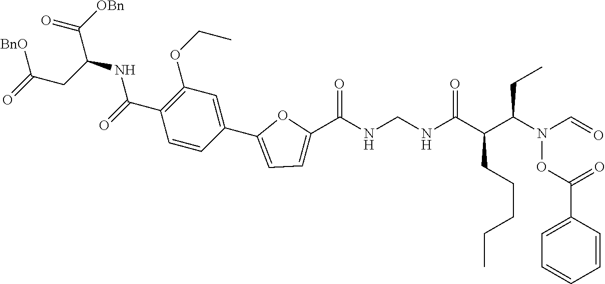 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccccc1]

Benzoic anhydride (278 mg, 1.230 mmol) was slowly added to a stirring solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (200 mg, 0.246 mmol) and Et₃N (171 μl, 1.230 mmol) in CH₃CN (1.6 mL) at rt. The resulting mixture was stirred for 1 h and then concentrated to dryness. Purification on Si (0-100% EtOAc/Hexanes) afforded the title compound as an off-white solid. (86 mg, 36.2% yield) MS (m/z) 917.4 (M+H)⁺

### Intermediate 7: Dibenzyl (3-(5-((((R)-2-((R)-1-(N-(benzoyloxy)formamido)propyl)heptanamido)methyl)carbamoyl)furan-2-yl)-5-ethoxyphenyl)phosphonate

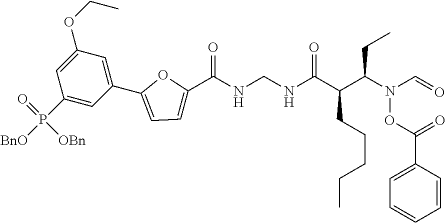 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccccc1]

To a solution containing dibenzyl (3-ethoxy-5-(5-((((R)-2-((R)-1-(N-hydroxyformamido)propyl)heptanamido)methyl)carbamoyl)furan-2-yl)phenyl)phosphonate (0.12 g, 0.16 mmol) in CH₃CN (3 mL) was added Et₃N (0.053 mL, 0.38 mmol) followed by benzoic anhydride (69.1 mg, 0.305 mmol). A clear solution formed and the reaction was stirred for 1 h at which time the reaction was concentrated. Purification on Si (50-100% EtOAc/Hexanes) afforded the title compound as an light brown solid. (90 mg, 70% yield) MS (m/z) 838.4 (M+H)⁺

### Intermediate 8: Dibenzyl (3-ethoxy-5-(5-((((R)-2-((R)-1-(N-(2-phenylacetoxy)formamido)propyl)heptanamido)methyl)carbamoyl)furan-2-yl)phenyl)phosphonate

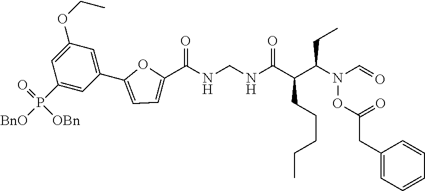 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)Cc1ccccc1]

To a solution containing dibenzyl (3-ethoxy-5-(5-((((R)-2-((R)-1-(N-hydroxyformamido)propyl)heptanamido)methyl)carbamoyl)furan-2-yl)phenyl)phosphonate (0.12 g, 0.16 mmol) in CH₃CN (3 mL) was added Et₃N (0.053 mL, 0.38 mmol) followed by 2-phenylacetyl chloride (47.2 mg, 0.305 mmol). A clear solution formed and the reaction was stirred for 1 h at which time the reaction was concentrated. Purification on Si (50-100% EtOAc/Hexanes) afforded the title compound as a light brown solid. (85 mg, 65% yield) MS (m/z) 852.4 (M+H)⁺

### Intermediate 9: (S)-dibenzyl 2-(2-ethoxy-4-(5-((((R)-2-((R)-1-(N-(2-phenylacetoxy)formamido)propyl)heptanamido)methyl)carbamoyl)furan-2-yl)benzamido)succinate

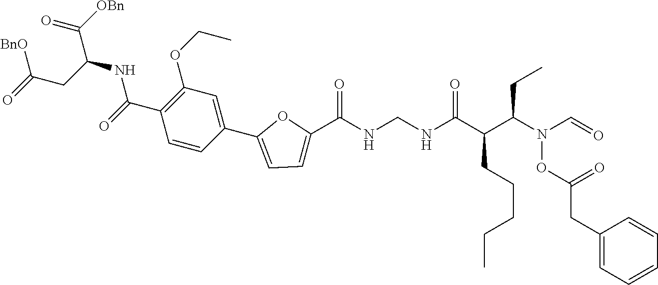 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)Cc1ccccc1]

To a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.325 g, 0.399 mmol) in anhydrous CH₃CN (2.66 mL) was added Et₃N (0.139 mL, 0.998 mmol), followed by dropwise addition of 2-phenylacetyl chloride (0.063 mL, 0.479 mmol). The reaction was stirred for 4 h and then concentrated to dryness. Purification on Si (0-75% EtOAc:Hexanes) afforded the title compound as a yellow oil. (0.075 g, 19% yield). MS (m/z) 931.4 (M+H)⁺

### Intermediate 10: (S)-dibenzyl 2-(2-ethoxy-4-(5-((((R)-2-((R)-1-(N-(pivaloyloxy)formamido)propyl)heptanamido)methyl)carbamoyl)furan-2-yl)benzamido)succinate

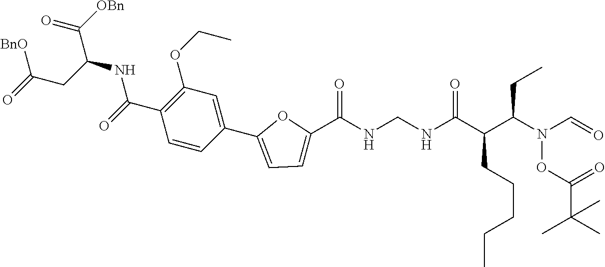 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)C(C)(C)C]

To a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.345 g, 0.425 mmol) in anhydrous CH₃CN (2.83 mL) was added Et₃N (0.296 mL, 2.124 mmol). The reaction was cooled to 0° C. and then pivalic anhydride (0.870 mL, 4.25 mmol) was added dropwise. The reaction was then stirred at rt for 3 h and concentrated. Purification on Si (0-100% EtOAc/Hexanes) afforded the title compound as a yellow solid. (0.170 g, 42% yield). MS (m/z) 897.4 (M+H)⁺

### Intermediate 11: (S)-dibenzyl 2-(2-(2-(benzyloxy)-2-oxoethoxy)-4-(5-((((R)-2-((R)-1-(N-((2-(benzyloxy)benzoyl)oxy)formamido)propyl)heptanamido)methyl)carbamoyl)furan-2-yl)benzamido)succinate

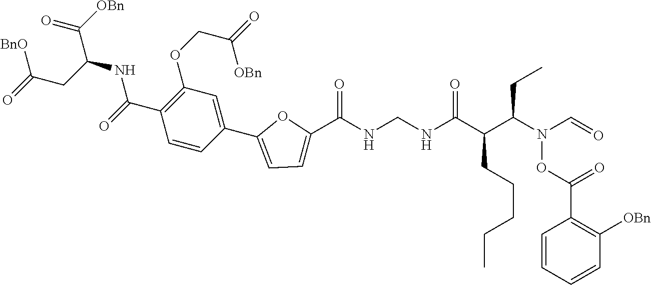 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccccc1OCc1ccccc1]

Et₃N (263 μL, 1.886 mmol) was added to a stirring solution of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (220 mg, 0.236 mmol) and 2-(benzyloxy)benzoyl chloride (233 mg, 0.943 mmol) in CH₃CN (1.5 mL). The resulting mixture was stirred at RT for 4 h. Water was added, and the organics were extracted using DCM (3×). The combined organics were dried over MgSO₄, filtered, and concentrated to dryness. Purification on Si (0-100% EtOAc/Hexanes) afforded the title compound as a tan solid. (148 mg, 55% yield). MS (m/z) 1143.7 (M+H)⁺

### Intermediate 12: (S)-dibenzyl 2-(4-(5-((((R)-2-((R)-1-(N-(benzoyloxy)formamido)propyl)heptanamido)methyl)carbamoyl)furan-2-yl)-2-(2-(benzyloxy)-2-oxoethoxy)benzamido)succinate

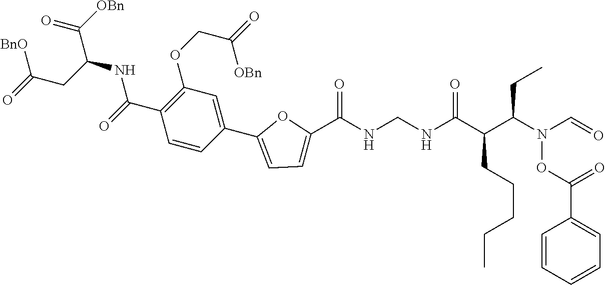 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccccc1]

Et₃N (170 μL, 1.222 mmol) was added to a stirring solution of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (228 mg, 0.244 mmol) and benzoic anhydride (276 mg, 1.222 mmol) in CH₃CN (1.6 mL). The resulting mixture was stirred for 15 min, and was then concentrated. Purification on Si (0-100% EtOAc/Hexanes) afforded the title compound as a tan solid. (153 mg, 60.4% yield) MS (m/z) 1037.6 (M+H)⁺

### Intermediate 13: (S)-dibenzyl 2-(2-(2-(benzyloxy)-2-oxoethoxy)-4-(5-((((R)-2-((R)-1-(N-((2-methylbenzoyl)oxy)formamido)propyl)heptanamido)methyl)carbamoyl)furan-2-yl)benzamido)succinate

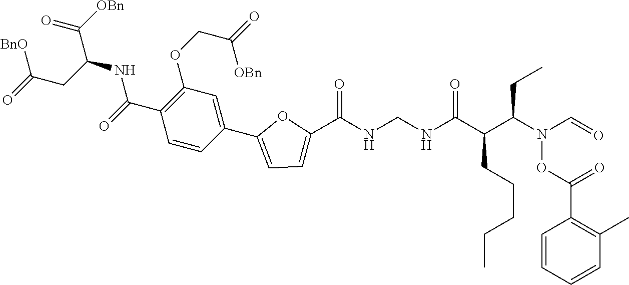 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccccc1C]

Et₃N (140 μl, 1.007 mmol) was added to a stirring solution of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (188 mg, 0.201 mmol) and 2-methylbenzoyl chloride (131 μl, 1.007 mmol) in CH₃CN (1.3 mL). The resulting mixture was stirred for 15 min and concentrated. Purification on Si (0-100% EtOAc/Hexanes) afforded the title compound as a tan solid. (157 mg, 70.4% yield) MS (m/z) 1052.0 (M+H)⁺

### Intermediate 14: Iodomethyl Pivalate

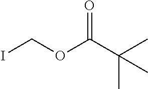

A mixture of chloromethyl pivalate (10 g, 66.4 mmol) and sodium iodide (11.94 g, 80 mmol) were stirred in Acetone (75 mL) for 5 h at RT. The mixture was evaporated, and ether was added. The mixture was filtered, and the filtrate was washed with saturated aqueous sodium thiosulfate followed by water. The combined organics were dried over MgSO₄, filtered, and concentrated. Purification on Si (0-10% EtOAc/Hexanes) afforded the title compound as an orange liquid. (6.9 g, 38.6% yield). Product does not ionize well—confirmed by NMR.

### Intermediate 15: (4R,5R)-10-(5-(3-(bis(benzyloxy)phosphoryl)-5-ethoxyphenyl)furan-2-yl)-4-ethyl-3-formyl-6,10-dioxo-5-pentyl-2-oxa-3,7,9-triazadecyl Pivalate

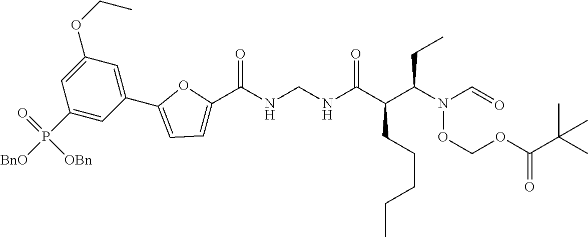 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OCOC(=O)C(C)(C)C]

To a solution containing dibenzyl (3-ethoxy-5-(5-((((R)-2-((R)-1-(N-hydroxyformamido)propyl)heptanamido)methyl)carbamoyl)furan-2-yl)phenyl)phosphonate (200 mg, 0.273 mmol) in THF (2 mL) was added DIPEA (0.143 mL, 0.818 mmol) and iodomethyl pivalate (132 mg, 0.545 mmol). The reaction was stirred for 12 h and concentrated. Purification on Si (0-100% EtOAc/Hexanes) afforded the title compound as a light brown solid. (118 mg, 51.1% yield). MS (m/z) 848.4 (M+H)⁺

### Intermediate 16: Benzyl ((4R,5R)-10-(5-(3-(bis(benzyloxy)phosphoryl)-5-ethoxyphenyl)furan-2-yl)-4-ethyl-3-formyl-6,10-dioxo-5-pentyl-2-oxa-3,7,9-triazadecyl) Hydrogen Phosphate

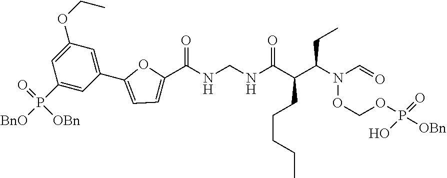 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OCOP(=O)(O)OCc1ccccc1]

To a solution containing dibenzyl (3-ethoxy-5-(5-((((R)-2-((R)-1-(N-hydroxyformamido)propyl)heptanamido)methyl)carbamoyl)furan-2-yl)phenyl)phosphonate (647 mg, 0.882 mmol) in THF (4 mL) at rt was added DIPEA (0.462 mL, 2.65 mmol) followed by dibenzyl (chloromethyl) phosphate (864 mg, 2.65 mmol) and sodium iodide (66.1 mg, 0.441 mmol). The reaction mixture was stirred for 4 days and was concentrated. Purification on Si (0-100% EtOAc/hexanes followed by 0-50% MeOH/EtOAc) afforded the title compound as a yellow solid. (314 mg, 38% yield) MS (m/z) 934.5 (M+H)⁺

### Intermediate 17: N—(((R)-2-((R)-1-(N-hydroxyformamido)propyl)heptanamido)methyl)-5-phenylfuran-2-carboxamide

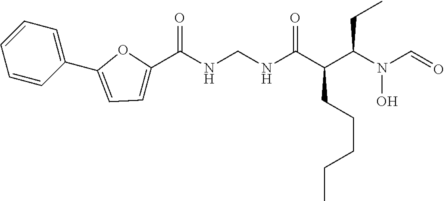 [SMILES: CCCCC[C@@H](C(=O)NCNC(=O)c1ccc(-c2ccccc2)o1)[C@@H](CC)N(O)C=O]

5-phenylfuran-2-carboxylic acid (0.73 g, 3.88 mmol), (R)—N-(aminomethyl)-2-((R)-1-(N-hydroxyformamido)propyl)heptanamide (1.006 g, 3.88 mmol), 2-(1H-benzo[d][1,2,3]triazol-1-yl)-1,1,3,3-tetramethylisouronium hexafluorophosphate(V) (1.618 g, 4.27 mmol) and DIPEA (2.033 mL, 11.64 mmol) were dissolved in DMF (30 mL) and stirred at rt for 2 h. The reaction mixture was diluted with EtOAc and water. The organic layer was washed with water (3×), dried over MgSO₄, filtered and concentrated. Purification on Si (0-60% EtOAc/Hexanes) afforded the title compound as a colorless oil. (0.86 g, 51% yield). MS (m/z) 430.2 (M+H)⁺

### Intermediate 18: (2S)-dibenzyl 2-(4-(5-((((2R)-2-((1R)-1-(N-((((benzyloxy)(hydroxy)phosphoryl)oxy)methoxy)formamido)propyl)heptanamido)methyl)carbamoyl)furan-2-yl)-2-(2-(benzyloxy)-2-oxoethoxy)benzamido)succinate

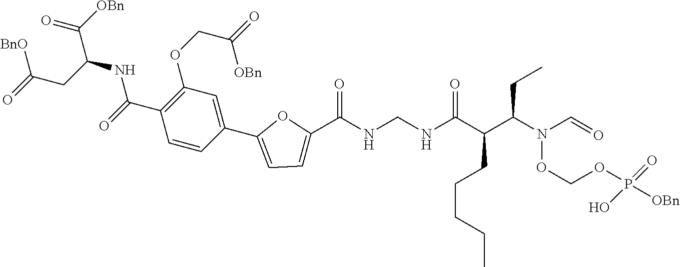 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OCOP(=O)(O)OCc1ccccc1]

DIPEA (112 μl, 0.643 mmol) was added to a stirring solution of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (200 mg, 0.214 mmol) and dibenzyl (chloromethyl) phosphate (210 mg, 0.643 mmol) in THF (1.4 mL). The resulting mixture was stirred at RT for 4 days and was concentrated. Purification on Si (0-40% MeOH/EtOAc) afforded product mixed with silica after concentration. The concentrate was taken up in EtOAc and was filtered through a syringe filter into a tared vial to give the title compound as a yellow solid. (106 mg, 44% yield) MS (m/z) 1133.7 (M+H)⁺

### Intermediate 19: (S)-dibenzyl 2-(4-(5-((((R)-2-((R)-1-(N-((2-acetoxybenzoyl)oxy)formamido) propyl)heptanamido)methyl)carbamoyl)furan-2-yl)-2-(2-(benzyloxy)-2-oxoethoxy)benzamido)succinate

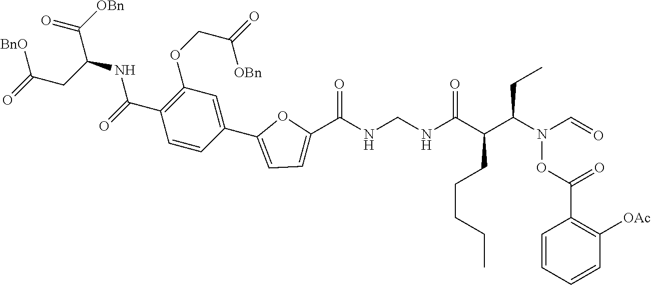 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccccc1OC(C)=O]

Et₃N (115 μl, 0.825 mmol) was added to a stirring solution of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (154 mg, 0.165 mmol) and 2-(chlorocarbonyl)phenyl acetate (164 mg, 0.825 mmol) in CH₃CN (1.1 mL). The resulting mixture was stirred for 15 min and concentrated. Purification on Si (0-100% EtOAc/Hexanes) afforded the title compound as a tan solid. (145 mg, 79% yield). MS (m/z) 1095.4 (M+H)⁺

### Intermediate 20: Dibenzyl (3-ethoxy-5-(5-((((R)-2-((R)-1-(N-((5-methyl-2-oxo-1,3-dioxol-4-yl)methoxy)formamido)propyl)heptanamido)methyl)carbamoyl)furan-2-yl)phenyl)phosphonate

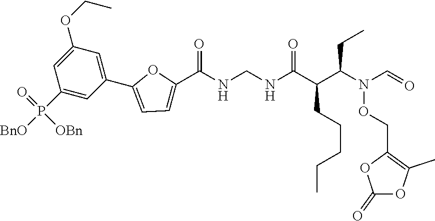 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OCc1oc(=O)oc1C]

To a solution containing dibenzyl (3-ethoxy-5-(5-((((R)-2-((R)-1-(N-hydroxyformamido)propyl)heptanamido)methyl)carbamoyl)furan-2-yl)phenyl)phosphonate (117 mg, 0.159 mmol) in THF (2 mL) was added potassium carbonate (66.1 mg, 0.478 mmol) and 4-(chloromethyl)-5-methyl-1,3-dioxol-2-one (71.1 mg, 0.478 mmol), followed by addition of sodium iodide (11.95 mg, 0.080 mmol). The reaction was stirred at RT overnight. The reaction was concentrated. Purification on Si (0-100% EtOAc/Hexanes) afforded the title compound as an off white solid. (70 mg, 52% yield). MS (m/z) 846.3 (M+H)⁺

### Intermediate 21: (S)-dibenzyl 2-(2-(2-(benzyloxy)-2-oxoethoxy)-4-(5-((((R)-2-((R)-1-(N-(nicotinoyloxy)formamido)propyl)heptanamido)methyl)carbamoyl)furan-2-yl)benzamido)succinate

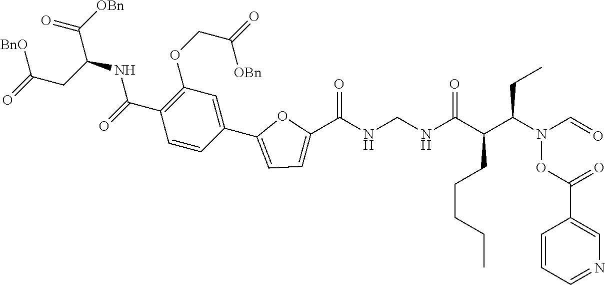 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1cccnc1]

To a rt suspension containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (100 mg, 0.107 mmol) in THF (2 mL) was added Et₃N (32.5 mg, 0.322 mmol) and nicotinoyl chloride (45.5 mg, 0.322 mmol). A dark yellow solution formed and the reaction was stirred for 12 h. The reaction mixture was concentrated. Purification on Si (0-100% EtOAc/Hexanes) afforded the title compound as a yellow solid. (67 mg, 60% yield). MS (m/z) 519.7 (M+H)^{+/}2

### Intermediate 22: (S)-dibenzyl 2-(2-(2-(benzyloxy)-2-oxoethoxy)-4-(5-(((4R,5R)-5-ethyl-6-formyl-10,10-dimethyl-3,8-dioxo-4-pentyl-7,9-dioxa-2,6-diazaundecyl)carbamoyl)furan-2-yl)benzamido)succinate

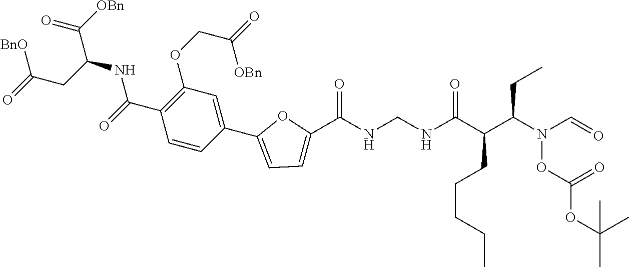 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)OC(C)(C)C]

Et₃N (116 μl, 0.831 mmol) was added to a stirring solution of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (155 mg, 0.166 mmol) and di-tert-butyl dicarbonate (181 mg, 0.831 mmol) in CH₃CN (1.1 mL). The resulting mixture was stirred for 15 min and concentrated. Purification on Si (0-100% EtOAc/Hexanes) afforded the title compound as a colorless solid. (100 mg, 58.3% yield). MS (m/z) 1033.9 (M+H)⁺

### Intermediate 23: (S)-5-(4-((1,4-dimethoxy-1,4-dioxobutan-2-yl)carbamoyl)-3-(2-methoxy-2-oxoethoxy)phenyl)furan-2-carboxylic Acid

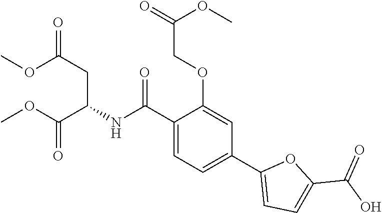 [SMILES: COC(=O)COc1cc(-c2ccc(C(=O)O)o2)ccc1C(=O)N[C@@H](CC(=O)OC)C(=O)OC]

### Step 1: (S)-dimethyl 2-(4-bromo-2-hydroxybenzamido)succinate

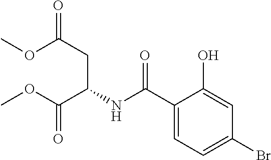 [SMILES: COC(=O)C[C@H](NC(=O)c1ccc(Br)cc1O)C(=O)OC]

DIPEA (79 mL, 464 mmol) was added to a stirring 0° C. mixture of 4-bromo-2-hydroxybenzoic acid (50.3 g, 232 mmol) and HATU (97 g, 255 mmol) in DMF (232 mL). The mixture was stirred for 10 min followed by addition of (S)-dimethyl 2-aminosuccinate hydrochloride (47.2 g, 239 mmol) in one portion. The resulting mixture was allowed to warm to RT and was stirred for 18 h at RT. Sat. aq. NH₄Cl was added, and the organics were extracted using EtOAc (3×). The combined organics were washed with sat. aq. NH₄Cl (3×), brine, dried over MgSO₄, filtered, and concentrated. Purification on Si (0-50% EtOAc/Hexanes) afforded the title compound as an orange gum. (81.76 g, 85% yield). MS (m/z) 360.1 (M+H)⁺

### Step 2: (S)-dimethyl 2-(4-bromo-2-(2-methoxy-2-oxoethoxy)benzamido)succinate

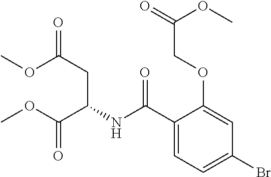 [SMILES: COC(=O)COc1cc(Br)ccc1C(=O)N[C@@H](CC(=O)OC)C(=O)OC]

Methyl 2-bromoacetate (47.3 mL, 499 mmol) and potassium carbonate (125 g, 908 mmol) were added to a stirring solution of (S)-dimethyl 2-(4-bromo-2-hydroxybenzamido)succinate (163.52 g, 454 mmol) in Acetone (649 mL). The resulting mixture was allowed to stir at RT for 3 h. Water was added, and the product was extracted into EtOAc (3×). The combined organics were washed with brine (1×), dried over MgSO₄, filtered, and concentrated. Purification on Si (0-70% EtOAc/Hexanes) afforded the title compound as an orange oil. (129.16 g, 65% yield). MS (m/z) 432.1 (M+H)⁺

### Step 3: (S)-dimethyl 2-(2-(2-methoxy-2-oxoethoxy)-4-(4,4,5,5-tetramethyl-1,3,2-dioxaborolan-2-yl)benzamido)succinate

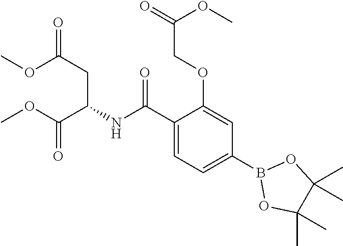

A solution of (S)-dimethyl 2-(4-bromo-2-(2-methoxy-2-oxoethoxy)benzamido)succinate (129.16 g, 299 mmol) in 1,4-Dioxane (1200 mL) was degassed with N₂ for 10 min. 4,4,4′,4′,5,5,5′,5′-octamethyl-2,2′-bi(1,3,2-dioxaborolane) (83 g, 329 mmol), PdCl₂(dppf)-CH₂Cl₂ (21.96 g, 26.9 mmol), and potassium acetate (88 g, 896 mmol) were added, and the mixture was degassed further for 5 min. The mixture was then heated to 90° C. for 12 h. Water was added, and the mixture was filtered through celite and the celite was washed with EtOAc. The combined organic phases were separated and the aqueous phase was extracted using EtOAc (2×). The combined organics were washed with brine, dried over MgSO₄, filtered, and concentrated onto silica. Purification on Si (0-100% EtOAc/Hexanes) afforded the title compound as an orange solid (143.49 g, 100% yield). MS (m/z) 480.2 (M+H)⁺

### Step 4: (S)-dimethyl 2-(4-(5-(tert-butoxycarbonyl)furan-2-yl)-2-(2-methoxy-2-oxoethoxy)benzamido)succinate

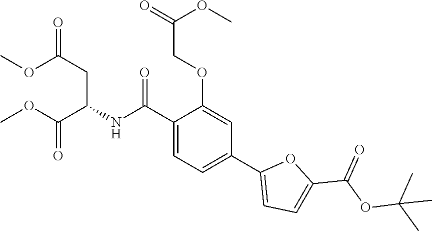 [SMILES: COC(=O)COc1cc(-c2ccc(C(=O)OC(C)(C)C)o2)ccc1C(=O)N[C@@H](CC(=O)OC)C(=O)OC]

N₂ was bubbled through a solution of (S)-dimethyl 2-(2-(2-methoxy-2-oxoethoxy)-4-(4,4,5,5-tetramethyl-1,3,2-dioxaborolan-2-yl)benzamido)succinate (70 g, 146 mmol) in 1,4-Dioxane (974 mL) for 30 min. Tert-butyl 5-bromofuran-2-carboxylate (39.7 g, 161 mmol) and PdCl₂(dppf)-CH₂Cl₂ (6.56 g, 8.03 mmol) were added followed by addition of sodium carbonate (438 mL, 438 mmol). The resulting mixture was heated to 50° C. under N₂ for 25 min. Water was added and the mixture was cooled to rt. EtOAc (200 mL) and water (100 mL) were added. The aqueous phase was extracted with EtOAc (3×). The combined extracts were dried over MgSO₄, filtered, and concentrated. Purification on Si (0-100% EtOAc/Hexanes) afforded the title compound as a tan solid. (43.75 g, 55% yield) MS (m/z) 520.2 (M+H)⁺

### Step 5: (S)-5-(4-((1,4-dimethoxy-1,4-dioxobutan-2-yl)carbamoyl)-3-(2-methoxy-2-oxoethoxy)phenyl)furan-2-carboxylic Acid

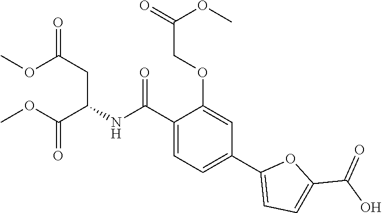 [SMILES: COC(=O)COc1cc(-c2ccc(C(=O)O)o2)ccc1C(=O)N[C@@H](CC(=O)OC)C(=O)OC]

TFA (348 mL, 4515 mmol) was added to a stirring solution of (S)-dimethyl 2-(4-(5-(tert-butoxycarbonyl)furan-2-yl)-2-(2-methoxy-2-oxoethoxy)benzamido)succinate (58.64 g, 113 mmol) in DCM (376 mL). The resulting mixture was allowed to stir for 18 h. The mixture was concentrated and azeotroped 3× with toluene to afford the title compound as a orange/brown gum. (55.6 g, 100% yield). MS (m/z) 464.1 (M+H)⁺

### Intermediate 25: Dibenzyl (3-(5-((((R)-2-((R)-1-(N-((2-(benzyloxy)benzoyl)oxy)formamido)propyl)heptanamido)methyl)carbamoyl)furan-2-yl)-5-ethoxyphenyl)phosphonate

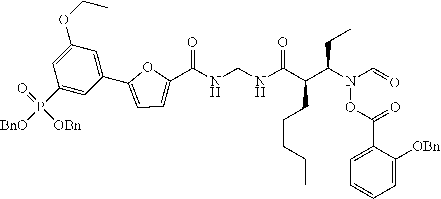 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccccc1OCc1ccccc1]

To a solution containing dibenzyl (3-ethoxy-5-(5-((((R)-2-((R)-1-(N-hydroxyformamido)propyl)heptanamido)methyl)carbamoyl)furan-2-yl)phenyl)phosphonate (0.22 g, 0.3 mmol) in THF (2 mL) was added DIPEA (0.219 mL, 1.2 mmol) and 2-(benzyloxy)benzoyl chloride (0.22 g, 0.90 mmol). The reaction mixture was stirred for 18 h. Purification on Si (0-100% EtOAc/Hexanes) afforded the title compound. (0.15 g, 51% yield) MS (m/z) 944.5 (M+H)⁺

### Intermediate 26: 3-((4R,5R)-10-(5-(3-(2-(benzyloxy)-2-oxoethoxy)-4-(((S)-1,4-bis(benzyloxy)-1,4-dioxobutan-2-yl)carbamoyl)phenyl)furan-2-yl)-4-ethyl-3-formyl-6,10-dioxo-5-pentyl-2-oxa-3,7,9-triazadecan-1-oyl)benzoic Acid

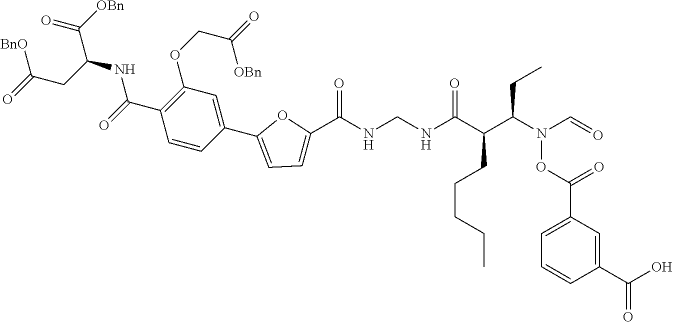 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1cccc(C(=O)O)c1]

To a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (100 mg, 0.107 mmol) in MeCN was added 4-methylmorpholine (32.5 mg, 0.322 mmol) followed by isophthalic acid (17.81 mg, 0.107 mmol), N1-((ethylimino)methylene)-N3,N3-dimethylpropane-1,3-diamine hydrochloride (24.66 mg, 0.129 mmol), and 3H-[1,2,3]triazolo[4,5-b]pyridin-3-ol (17.51 mg, 0.129 mmol). The resulting mixture was stirred for 1 h and was then concentrated. Purification by reverse phase HPLC afforded the title compounds as a colorless solid. (100 mg, 86% yield)

### Intermediate 27: (S)-dibenzyl 2-(2-(2-(benzyloxy)-2-oxoethoxy)-4-(5-((((R)-2-((R)-1-(N-((1-methylcyclopropanecarbonyl)oxy)formamido)propyl)heptanamido)methyl)carbamoyl)furan-2-yl)benzamido)succinate

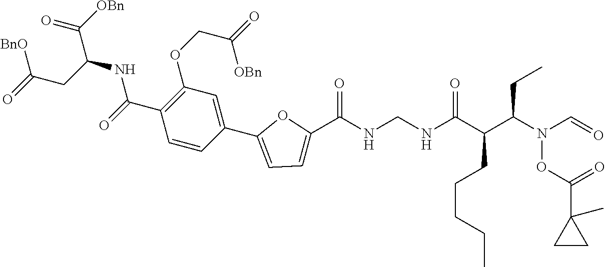 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)C1(C)CC1]

DIPEA (84 μl, 0.482 mmol) was added to a stirring mixture of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol), 1-methylcyclopropanecarboxylic acid (24.14 mg, 0.241 mmol), and HATU (73.4 mg, 0.193 mmol) in MeCN (643 μl). The mixture was stirred for 1 h and was then concentrated to dryness. Purification on Si (0-100% EtOAc/Hex) afforded the title compound as a yellow solid. (142 mg, 87% yield) MS (m/z) 1015.5 (M+H)⁺

### Intermediate 28: (2S)-dibenzyl 2-(2-(2-(benzyloxy)-2-oxoethoxy)-4-(5-((((2R)-2-((1R)-1-(N-((2-methylcyclopropanecarbonyl)oxy)formamido)propyl)heptanamido)methyl)carbamoyl)furan-2-yl)benzamido)succinate

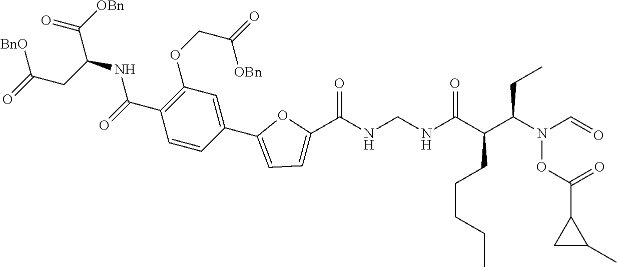 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)C1CC1C]

DIPEA (84 μl, 0.482 mmol) was added to a stirring mixture of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol), 2-methylcyclopropanecarboxylic acid (cis and trans) (24.14 mg, 0.241 mmol), and HATU (73.4 mg, 0.193 mmol) in MeCN (643 μl). The mixture was stirred for 1 h and was then concentrated to dryness. Purification on Si (0-100% EtOAc/Hex) afforded the title compound as a yellow solid. (163 mg, 100% yield) MS (m/z) 1015.6 (M+H)⁺

### Intermediate 29: (S)-dibenzyl 2-(2-(2-(benzyloxy)-2-oxoethoxy)-4-(5-((((R)-2-((R)-1-(N-((4-methoxybenzoyl)oxy)formamido)propyl)heptanamido)methyl)carbamoyl)furan-2-yl)benzamido)succinate

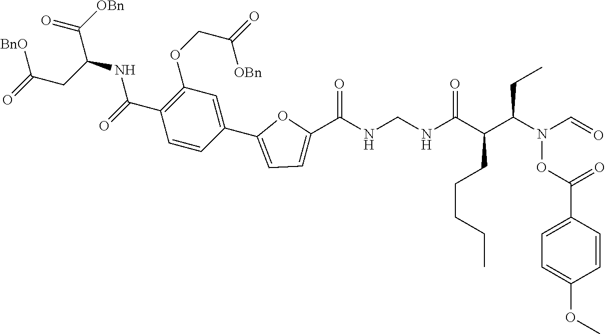 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccc(OC)cc1]

DIPEA (84 μl, 0.482 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) and 4-methoxybenzoyl chloride (82 mg, 0.482 mmol) in MeCN (643 μl). The mixture was stirred for 1 h and was then concentrated to dryness. Purification on Si (0-100% EtOAc/Hex) afforded the title compound as a yellow solid. (150 mg, 87% yield). MS (m/z) 1067.6 (M+H)⁺

### Intermediate 30: (S)-dibenzyl 2-(2-(2-(benzyloxy)-2-oxoethoxy)-4-(5-(((6R,7R)-6-ethyl-5-formyl-3,8-dioxo-7-pentyl-4-oxa-2,5,9-triazadecan-10-yl)carbamoyl)furan-2-yl)benzamido)succinate

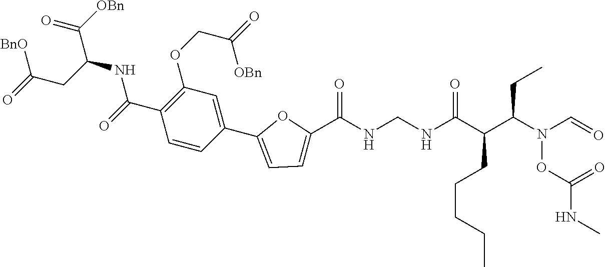 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)NC]

TEA (132 μl, 0.949 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (177 mg, 0.190 mmol) and methylcarbamic chloride (89 mg, 0.949 mmol) in MeCN (1265 μl). The resulting mixture was stirred for 18 h, and was then evaporated. Purification on Si (0-100% EtOAc/Hex) afforded the title compound as a tan solid. (94 mg, 50% yield). MS (m/z) 990.5 (M+H)⁺

### Intermediate 31: (S)-dibenzyl 2-(4-(5-((((R)-2-((R)-1-(N-((2-aminobenzoyl)oxy)formamido)propyl)heptanamido)methyl)carbamoyl)furan-2-yl)-2-(2-(benzyloxy)-2-oxoethoxy)benzamido)succinate

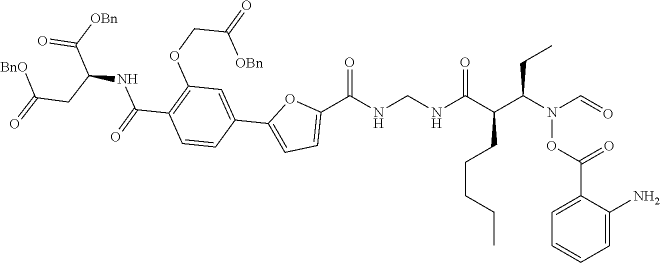 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccccc1N]

DMAP (2.62 mg, 0.021 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (200 mg, 0.214 mmol) and 1H-benzo[d][1,3]oxazine-2,4-dione (45.5 mg, 0.279 mmol), and DIPEA (74.9 μl, 0.429 mmol) in DMF (715 μl). The reaction mixture was heated to 60° C. for 1 hr and was then stirred for 18 h at RT. Silica gel was added to the mixture, and the solvent was evaporated. Purification by Si (0-100% EtOAc/Hex) afforded the tile compound as a yellow solid. (200 mg, 89% yield). MS (m/z) 1052.8 (M+H)⁺

### Intermediate 32: (S)-dibenzyl 2-(2-(2-(benzyloxy)-2-oxoethoxy)-4-(5-((((R)-2-((R)-1-(N-((2-(methylamino)benzoyl)oxy)formamido)propyl)heptanamido)methyl)carbamoyl)furan-2-yl)benzamido)succinate

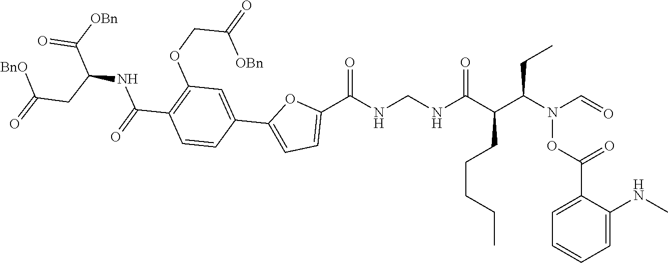 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccccc1NC]

DMAP (2.62 mg, 0.021 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (200 mg, 0.214 mmol) and 1-methyl-1H-benzo[d][1,3]oxazine-2,4-dione (49.4 mg, 0.279 mmol), and DIPEA (74.9 μl, 0.429 mmol) in DMF (715 μl). The resulting mixture was heated to 60° C. for 1 h and was then stirred for 18 h at RT. Silica gel was added to the mixture, and the solvent was evaporated. Purification by Si (0-100% EtOAc/Hex) afforded the title compound as a yellow gum. (161 mg, 70% yield). MS (m/z) 1066.8 (M+H)⁺

### Intermediate 33: (S)-dibenzyl 2-(2-(2-(benzyloxy)-2-oxoethoxy)-4-(5-((((R)-2-((R)-1-(N-((2-ethylbutanoyl)oxy)formamido)propyl)heptanamido)methyl)carbamoyl)furan-2-yl)benzamido)succinate

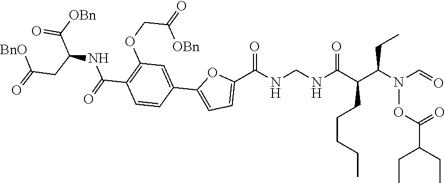 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)C(CC)CC]

N-methylmorpholine (106 μl, 0.965 mmol) was added to a solution containing (R)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (300 mg, 0.322 mmol), 2-ethylbutanoic acid (60.9 μl, 0.482 mmol), and HATU (147 mg, 0.386 mmol). The resulting mixture was stirred for 1 h and was then concentrated. Purification using Si (0-100% EtOAc/Hex) afforded the title compound. (308 mg, 92% yield). MS (m/z) 1031.6 (M+H)⁺

### Intermediate 34: (S)-dibenzyl 2-(2-(2-(benzyloxy)-2-oxoethoxy)-4-(5-((((R)-2-((R)-1-(N-((3,5-dimethylisoxazole-4-carbonyl)oxy)formamido)propyl)heptanamido)methyl)carbamoyl)furan-2-yl)benzamido)succinate

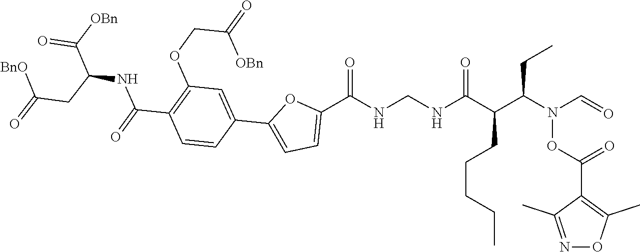 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1c(C)noc1C]

DIPEA (84 μl, 0.482 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) and 3,5-dimethylisoxazole-4-carboxylic acid (24.96 mg, 0.177 mmol), and HATU (73.4 mg, 0.193 mmol) in DMF (1072 μl). The resulting mixture was stirred for 2 days at RT. Purification of the crude reaction mixture by reverse phase HPLC afforded the title compound as a colorless solid. (98 mg, 52% yield). MS (m/z) 1056.6 (M+H)⁺

### Intermediate 35: (S)-dibenzyl 2-(2-(2-(benzyloxy)-2-oxoethoxy)-4-(5-((((R)-2-((R)-1-(N-((2,4-dimethylnicotinoyl)oxy)formamido)propyl)heptanamido)methyl)carbamoyl)furan-2-yl)benzamido)succinate

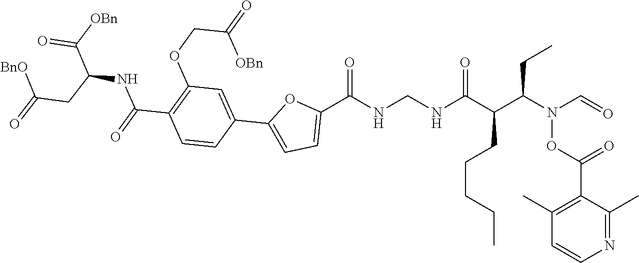 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1c(C)ccnc1C]

DIPEA (84 μl, 0.482 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) and 2,4-dimethylnicotinic acid (26.7 mg, 0.177 mmol), and HATU (73.4 mg, 0.193 mmol) in DMF (1072 μl). The reaction mixture was stirred for 2 days at RT. Purification of the crude reaction mixture by reverse phase HPLC afforded the title compound as a colorless solid. (76 mg, 44% yield). MS (m/z) 1067.1 (M+H)⁺

### Intermediate 36: (S)-dibenzyl 2-(2-(2-(benzyloxy)-2-oxoethoxy)-4-(5-((((R)-2-((R)-1-(N-(2,2-diphenylacetoxy)formamido)propyl)heptanamido)methyl)carbamoyl)furan-2-yl)benzamido)succinate

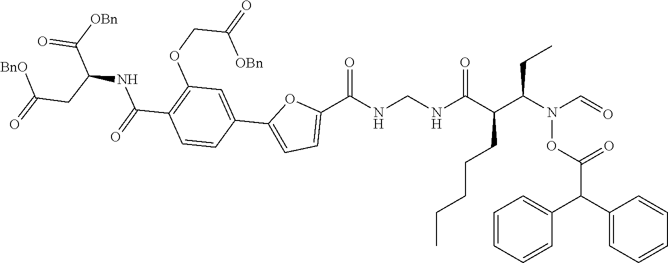 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)C(c1ccccc1)c1ccccc1]

DIPEA (84 μl, 0.482 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) and 2,2-diphenylacetic acid (34.1 mg, 0.161 mmol), and HATU (73.4 mg, 0.193 mmol) in DMF (1072 μl). The resulting mixture was stirred for 2 days at RT. Purification of the crude reaction mixture by reverse phase HPLC afforded the title compound as a pale yellow solid. (83 mg, 46% yield). MS (m/z) 1127.2 (M+H)⁺

### Intermediate 37: (S)-dibenzyl 2-(2-(2-(benzyloxy)-2-oxoethoxy)-4-(5-((((R)-2-((R)-1-(N-((2-isopropylbenzoyl)oxy)formamido)propyl)heptanamido)methyl)carbamoyl)furan-2-yl)benzamido)succinate

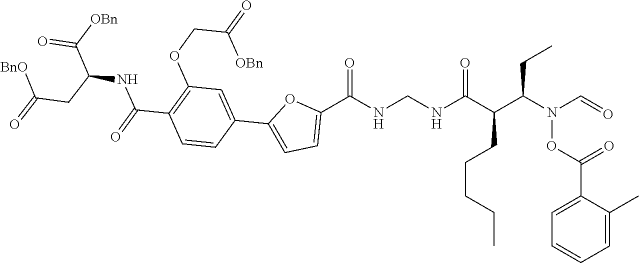 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccccc1C]

DIPEA (84 μl, 0.482 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) and 2-isopropylbenzoic acid (29.0 mg, 0.177 mmol), and HATU (73.4 mg, 0.193 mmol) in DMF (1072 μl). The resulting mixture was heated to 50° C. for 1 hr and was then stirred for 18 h at RT. Purification of the crude reaction mixture by reverse phase HPLC afforded the title compound as an yellow solid. (112 mg, 65% yield). MS (m/z) 1079.9 (M+H)⁺

### Intermediate 38: (S)-dibenzyl 2-(2-(2-(benzyloxy)-2-oxoethoxy)-4-(5-(((4R,5R)-5-ethyl-6-formyl-10,10-dimethyl-3,8-dioxo-4-pentyl-7-oxa-2,6,9-triazaundecyl)carbamoyl)furan-2-yl)benzamido)succinate

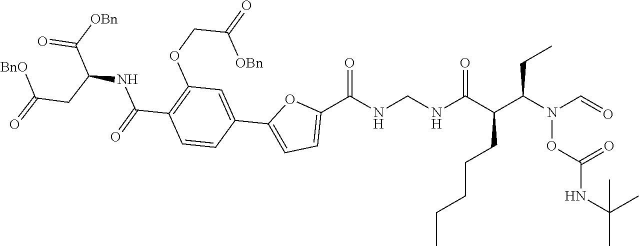 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)NC(C)(C)C]

2-isocyanato-2-methylpropane (80 mg, 0.804 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) and TEA (81 mg, 0.804 mmol) in DCM (322 μl). The resulting mixture was stirred for 18 h and evaporated. Purification of the crude reaction mixture by reverse phase HPLC afforded the title compound as a white solid. (125 mg, 75% yield). MS (m/z) 1032.9 (M+H)⁺

### Intermediate 39: (S)-dibenzyl 2-(2-(2-(benzyloxy)-2-oxoethoxy)-4-(5-(((4R,5R)-5-ethyl-6-formyl-10-methyl-3,8-dioxo-4-pentyl-7-oxa-2,6,9-triazaundecyl)carbamoyl)furan-2-yl)benzamido)succinate

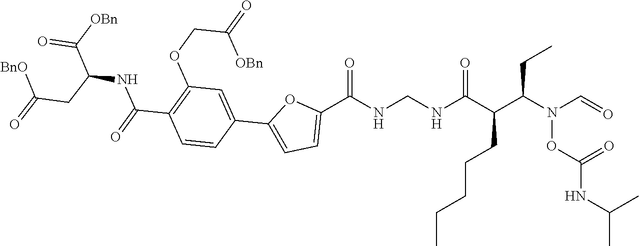 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)NC(C)C]

2-isocyanatopropane (68.4 mg, 0.804 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) and TEA (81 mg, 0.804 mmol) in DCM (322 μl). The resulting mixture was stirred for 18 h and evaporated. Purification of the crude reaction mixture by reverse phase HPLC afforded the title compound as a white solid. (112 mg, 68% yield). MS (m/z) 1018.8 (M+H)⁺

### Intermediate 40: 2-((4R,5R)-10-(5-(3-(bis(benzyloxy)phosphoryl)-5-ethoxyphenyl)furan-2-yl)-4-ethyl-3-formyl-6,10-dioxo-5-pentyl-2-oxa-3,7,9-triazadecan-1-oyl)phenyl Dimethylcarbamate

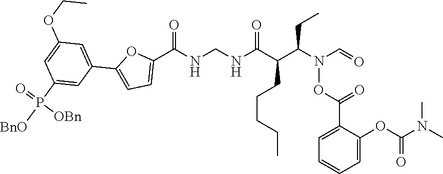 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccccc1OC(=O)N(C)C]

To a solution containing dibenzyl (3-ethoxy-5-(5-((((R)-2-((R)-1-(N-hydroxyformamido)propyl)heptanamido)methyl)carbamoyl)furan-2-yl)phenyl)phosphonate (150 mg, 0.204 mmol) and 2-((dimethylcarbamoyl)oxy)benzoic acid (47.0 mg, 0.225 mmol) in DMF (2 mL) was added HATU (93 mg, 0.245 mmol) and DIPEA (0.107 mL, 0.613 mmol). The reaction was stirred at RT for 2 h. Reaction was diluted with water (10 mL) and extracted with EtOAc. Organic phase was combined and washed with water and brine and passed through hydrophobic frit and concentrated. Purification by Si (0-100% EtOAc/Hex) afforded the title compound as a dark yellow oil. (106 mg, 56% yield). MS (m/z) 925.5 (M+H)⁺

### Intermediate 41: 2-((dimethylcarbamoyl)oxy)benzoic Acid

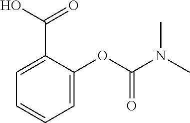

### Step 1: Methyl 2-((dimethylcarbamoyl)oxy)benzoate

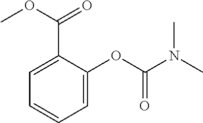

To a solution containing methyl 2-hydroxybenzoate (0.085 mL, 0.657 mmol) and potassium carbonate (454 mg, 3.29 mmol) in MeCN (2 mL) was added dimethylcarbamic chloride (0.605 mL, 6.57 mmol). Reaction was stirred at 60° C. for 18 h. The reaction was filtered and washed with MeCN. The filtrate was concentrated, dilute with MeCN, filter again and concentrated to afford title compound as a clear oil. (194 mg, ˜100% yield). MS (m/z) 224.2 (M+H)⁺

### Step 2: 2-((dimethylcarbamoyl)oxy)benzoic Acid

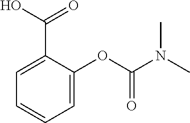

To a solution containing methyl 2-((dimethylcarbamoyl)oxy)benzoate (194.7 mg, 0.872 mmol) in MeOH (2 mL) and water (0.5 mL) was added LiOH (47.2 mg, 1.9 mmol). The reaction was stirred at RT for 18 hr. Additional LiOH (15 mg) was added and stirred for 4 h. The reaction was concentrated and diluted with EtOAc (5 mL), and acidified with 1N HCl. Extracted with EtOAc and the combined organic layer was passed through a hydrophobic frit and concentrated to afford the title compound as a colorless solid. (144 mg, 100% yield).

### Intermediate 42: (S)-dibenzyl 2-(2-(2-(benzyloxy)-2-oxoethoxy)-4-(5-((((R)-2-((R)-1-(N-((2-((dimethylcarbamoyl)oxy)benzoyl)oxy)formamido)propyl)heptanamido)methyl)carbamoyl)furan-2-yl)benzamido)succinate

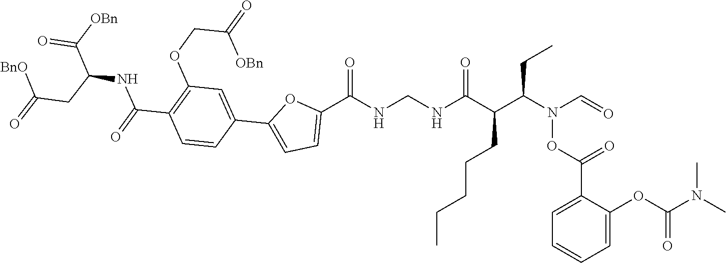 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccccc1OC(=O)N(C)C]

To a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (200 mg, 0.214 mmol) and 2-((dimethylcarbamoyl)oxy)benzoic acid (49.3 mg, 0.236 mmol) in DMF (2 mL) was added HATU (98 mg, 0.257 mmol) and DIPEA (0.112 mL, 0.643 mmol). The reaction was stirred at RT for 2 h. Diluted with water (10 mL) and extracted with EtOAc (10 mL). The organic phase was washed with water and brine, passed through a hydrophobic frit, and concentrated. Purification by Si (0-100% EtOAc/Hex) afforded the title compound as a yellow oil. (206 mg, 86% yield). MS (m/z) 1124.7 (M+H)⁺

### Intermediate 43: (S)-2-(4-(5-((((R)-2-((R)-1-(N-((1-naphthoyl)oxy)formamido)propyl)heptanamido)methyl)carbamoyl)furan-2-yl)-2-(carboxymethoxy)benzamido)succinic Acid

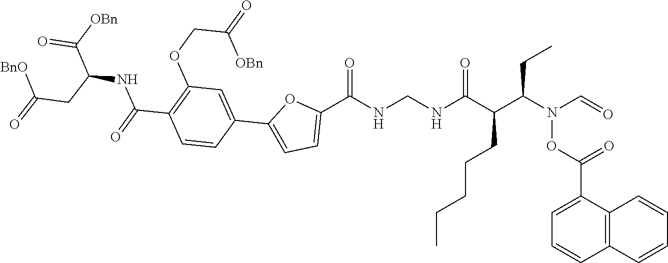 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1cccc2ccccc12]

To a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) in DCM (3 mL) at 0° C. was added TEA (0.067 mL, 0.482 mmol) followed by addition of 1-naphthoyl chloride (0.051 mL, 0.322 mmol). The reaction was mixture was stirred at RT for 2 h. Additional 1-naphthoyl chloride (0.051 mL, 0.322 mmol) was added and stirred for 18 h. Reaction was concentrated. Purification by Si (0-100% EtOAc/Hex) afforded the title compound as a clear oil. (53 mg, 30% yield). MS (m/z) 1087.5 (M+H)⁺

### Intermediate 44: (S)-dibenzyl 2-(4-(5-((((R)-2-((R)-1-(N-((2-naphthoyl)oxy)formamido)propyl)heptanamido)methyl)carbamoyl)furan-2-yl)-2-(2-(benzyloxy)-2-oxoethoxy)benzamido)succinate

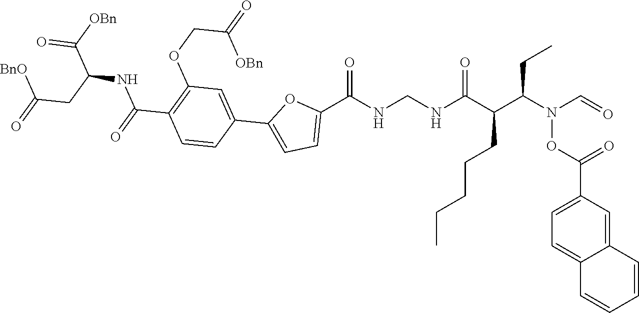 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccc2ccccc2c1]

To a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) in DCM (3 mL) at 0° C. was added TEA (0.067 mL, 0.482 mmol) followed by addition of 2-naphthoyl chloride (61.3 mg, 0.322 mmol). The reaction was mixture was stirred at RT for 2 h. Additional 2-naphthoyl chloride (61.3 mg, 0.322 mmol) was added and stirred for 18 h. Reaction was concentrated. Purification by Si (0-100% EtOAc/Hex) afforded the title compound as a clear oil. (80 mg, 46% yield). MS (m/z) 1087.5 (M+H)⁺

### Intermediate 45: (S)-dibenzyl 2-(2-(2-(benzyloxy)-2-oxoethoxy)-4-(5-((((R)-2-((R)-1-(N-((4-(morpholinomethyl)benzoyl)oxy)formamido)propyl)heptanamido)methyl)carbamoyl)furan-2-yl)benzamido)succinate

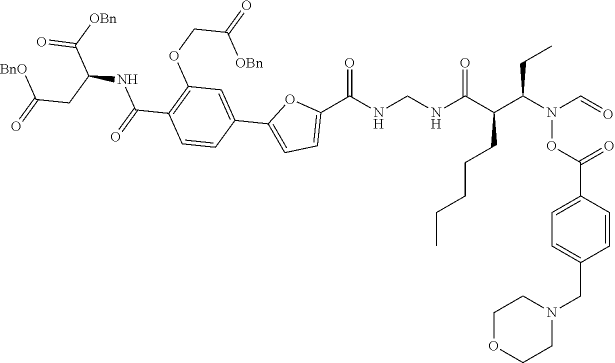 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccc(CN2CCOCC2)cc1]

To a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (0.66 g, 0.707 mmol) in MeCN (6 mL) was added HATU (0.403 g, 1.061 mmol), 4-(morpholinomethyl)benzoic acid, Hydrochloride (0.182 g, 0.707 mmol) and DIPEA (0.494 mL, 2.83 mmol). The reaction was stirred for 18 h and the reaction mixture was poured into NH4Cl aq., extracted with EtOAc, dried over MgSO4, filtered and concentrated onto SiO2. Purification by Si (0-100% EtOAc/Hex) afforded the title compound as a brown oil. (190 mg, 24% yield). MS (m/z) 1036.6 (M+H)⁺

### Intermediate 46: (S)-dibenzyl 2-(2-(2-(benzyloxy)-2-oxoethoxy)-4-(5-(((4R,5R)-5-ethyl-6-formyl-10-methyl-3,8-dioxo-4-pentyl-10-phenyl-7-oxa-2,6,9-triazaundecyl)carbamoyl)furan-2-yl)benzamido)succinate

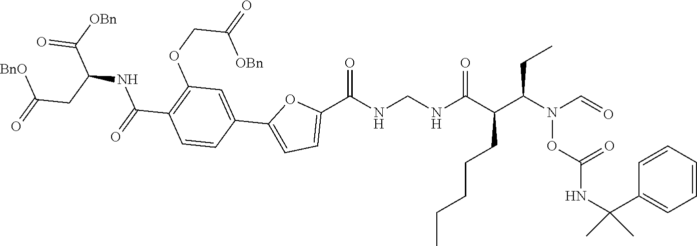 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)NC(C)(C)c1ccccc1]

To a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) and TEA (112 μl, 0.804 mmol) in DCM (322 μl) was added (2-isocyanatopropan-2-yl)benzene (130 mg, 0.804 mmol). The resulting mixture was stirred for 3 h and concentrated. Purification by Si (0-100% EtOAc/Hex) afforded the title compound as a clear oil. (110 mg, 63% yield). MS (m/z) 1094.5 (M+H)⁺

### Intermediate 47: (2S)-dibenzyl 2-(2-(2-(benzyloxy)-2-oxoethoxy)-4-(5-((((2R)-2-((1R)-1-(N—(((((S)-1-methoxy-1-oxopropan-2-yl)amino)(phenoxy)phosphoryl)oxy)formamido)propyl)heptanamido)methyl)carbamoyl)furan-2-yl)benzamido)succinate

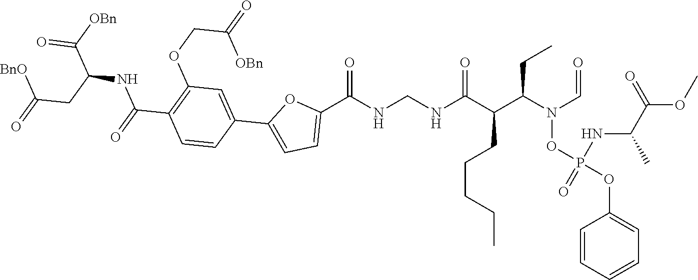 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OP(=O)(N[C@@H](C)C(=O)OC)Oc1ccccc1]

(S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (0.366 g, 0.392 mmol) and TEA (0.055 mL, 0.392 mmol) in DCM (2 mL) was added drop wise to a solution of phenyl phosphorodichloridate (0.059 mL, 0.392 mmol) in DCM (2 mL) at −60° C. After stirring for 0.5 hr at −60° C., (S)-methyl 2-aminopropanoate, Hydrochloride (0.055 g, 0.392 mmol) was added followed by drop wise addition of a solution of TEA (0.055 mL, 0.392 mmol) (2×) in DCM (2 mL) and the reaction allowed to warm up to RT over 3 h. Water was added and the reaction was extracted with DCM. The organic layer was passed through a hydrophobic cartridge and concentrated. Purification by Si (0-70% EtOAc/Hex) afforded the title compound as a clear oil. (45 mg, 10% yield). MS (m/z) 587.9 (M/2+H)⁺

### Intermediate 48: (S)-dibenzyl 2-(2-(2-(benzyloxy)-2-oxoethoxy)-4-(5-((((R)-2-((R)-1-(N-((2-fluoro-6-methylbenzoyl)oxy)formamido)propyl)heptanamido)methyl)carbamoyl)furan-2-yl)benzamido)succinate

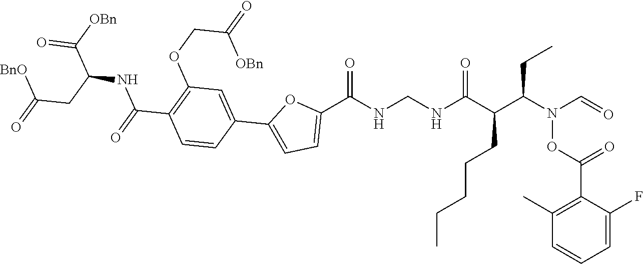 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1c(C)cccc1F]

N-methylmorpholine (70.7 μl, 0.643 mmol) was added to a solution containing (R)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (200 mg, 0.214 mmol) and 2-fluoro-6-methylbenzoyl chloride (55.5 mg, 0.322 mmol) in MeCN (2144 μl). The resulting mixture was stirred for 1 h and was then concentrated. Purification by Si (0-100% EtOAc/Hex) afforded the title compound as a clear solid. (113 mg, 47% yield). MS (m/z) 1069.5 (M+H)⁺

### Intermediate 49: (S)-dibenzyl 2-(2-(2-(benzyloxy)-2-oxoethoxy)-4-(5-((((R)-2-((R)-1-(N-((4-methoxy-2-methylbenzoyl)oxy)formamido)propyl)heptanamido)methyl)carbamoyl)furan-2-yl)benzamido)succinate

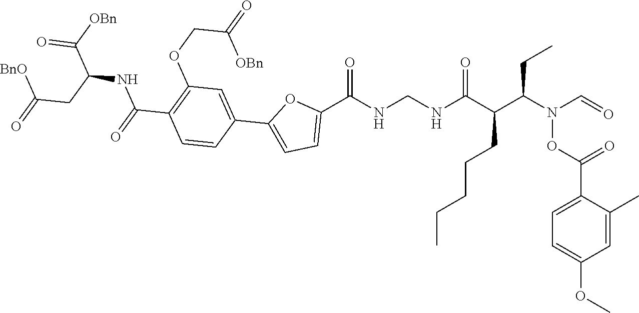 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccc(OC)cc1C]

Methanesulfonyl chloride (0.014 mL, 0.177 mmol) was added to a solution containing 4-methoxy-2-methylbenzoic acid (0.027 g, 0.161 mmol) and 1-methylimidazole (0.026 mL, 0.322 mmol) in DCM (1.6 mL) at 0° C. After stirring for 30 mins at 0° C., (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (0.15 g, 0.161 mmol), was added and the reaction was stirred at RT for 18 h. Water was added and the reaction mixture was partitioned between water and DCM. The organic layer was passed through a hydrophobic cartridge and concentrated. Purification by Si (0-80% EtOAc/Hex) afforded the title compound as a clear solid. (91 mg, 52% yield). MS (m/z) 541.3 (M/2+H)⁺

### Intermediate 50: (S)-dibenzyl 2-(2-(2-(benzyloxy)-2-oxoethoxy)-4-(5-((((R)-2-((R)-1-(N-((3-methylisonicotinoyl)oxy)formamido)propyl)heptanamido)methyl)carbamoyl)furan-2-yl)benzamido)succinate

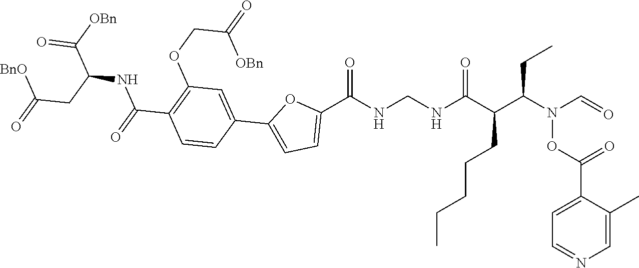 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccncc1C]

Methanesulfonyl chloride (0.014 mL, 0.177 mmol) was added to a solution containing 3-methylisonicotinic acid (0.022 g, 0.161 mmol) and 1-methylimidazole (0.026 mL, 0.322 mmol) in DCM (1.072 mL) at 0° C. After stirring at 0° C. for 30 mins, (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (0.15 g, 0.161 mmol) was added and the reaction was allowed to warm up to RT and stirred for 2.5 days. Water was added and the reaction was partitioned between water and DCM. The organic layer was passed through a hydrophobic cartridge and concentrated. Purification by Si (0-100% EtOAc/Hex) afforded the title compound as a colorless foam. (78 mg, 46% yield). MS (m/z) 526.8 (M/2+H)⁺

### Intermediate 51: (S)-dibenzyl 2-(2-(2-(benzyloxy)-2-oxoethoxy)-4-(5-((((R)-2-((R)-1-(N-((2-methyl-4-(morpholinomethyl)benzoyl)oxy)formamido)propyl)heptanamido)methyl)carbamoyl)furan-2-yl)benzamido)succinate

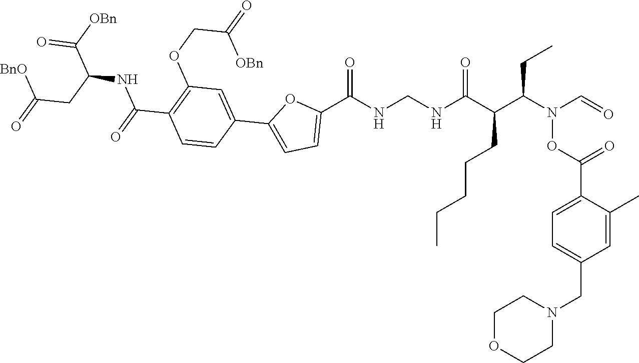 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccc(CN2CCOCC2)cc1C]

To a solution containing 2-methyl-4-(morpholinomethyl)benzoic acid, Hydrochloride (0.261 g, 0.962 mmol) in MeCN (2 mL) was added HATU (0.499 g, 1.312 mmol) and DIPEA (0.611 mL, 3.50 mmol). The reaction was stirred for 15 min at which time, (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (0.816 g, 0.875 mmol) was added in MeCN (2 mL). The reaction was stirred 18 h. Reaction mixture was concentrated onto celite. Purification by Si (0-100% EtOAc/Hex) afforded the title compound as a brown oil. (438 mg, 44% yield). MS (m/z) 1150.6 (M+H)⁺

### Intermediate 52: 2-methyl-4-(morpholinomethyl)benzoic Acid, Hydrochloride

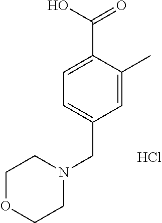 [SMILES: Cc1cc(CN2CCOCC2)ccc1C(=O)O ; Cl]

### Step 1: Ethyl 4-(bromomethyl)-2-methylbenzoate

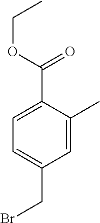

To a solution containing ethyl 2,4-dimethylbenzoate (10 g, 56.1 mmol) in carbontetrachloride (100 mL) was added NBS (10.69 g, 60.0 mmol) and benzoyl peroxide (0.166 g, 0.673 mmol). The reaction was stirred at 80° C. for 40 h. The mixture was filtered and washed with DCM. The resulting solution was concentrated to afford a yellow oil. Oil was heated to 140° C. for 90 min. The resulting oil was cooled and stirred with hexanes to induce precipitation of the undesired lactone which was filtered and washed with hexanes. Further precipitation was observed upon concentration. The solution was filtered again and washed with hexanes. Filtrate was concentrated onto celite. Purification by Si (0-10% EtOAc/Hex) afforded the title compound as a colorless oil. (4.5 g, 31% yield). MS (m/z) 257.0 (M+H)⁺

### Step 2: Ethyl 2-methyl-4-(morpholinomethyl)benzoate

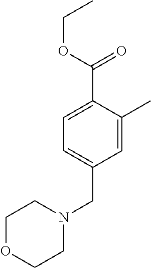 [SMILES: CCOC(=O)c1ccc(CN2CCOCC2)cc1C]

To a solution containing morpholine (2.264 mL, 26.3 mmol) in EtOH (50 mL) was added potassium iodide (0.581 g, 3.50 mmol) and potassium carbonate (6.05 g, 43.8 mmol) followed by ethyl 4-(bromomethyl)-2-methylbenzoate (4.5 g, 17.50 mmol). The reaction was stirred at RT for 18 h. Reaction mixture was diluted with water and extracted with EtOAc, dried over MgSO4, filtered and concentrated onto SiO₂. Purification by Si (0-75% EtOAc/Hex) afforded the title compound as a colorless oil. (2.7 g, 59% yield). MS (m/z) 264.1 (M+H)⁺

### Step 3: 2-methyl-4-(morpholinomethyl)benzoic Acid, Hydrochloride

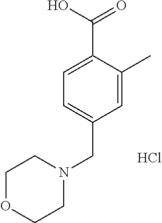 [SMILES: Cc1cc(CN2CCOCC2)ccc1C(=O)O ; Cl]

ethyl 2-methyl-4-(morpholinomethyl)benzoate (2.71 g, 10.29 mmol) was taken up in sodium hydroxide (15.44 mL, 30.9 mmol) and stirred for 18 h. Hydrochloric acid (10.29 mL, 61.7 mmol) was added and the reaction was chilled to 0° C. and stirred for 1 hr. Reaction mixture was filtered, washed with hexanes, and dried to afford the title compound as a colorless solid. (2.43 g, 87% yield). MS (m/z) 236.1 (M+H)⁺

### Intermediate 53: (S)-dibenzyl 2-(2-(2-(benzyloxy)-2-oxoethoxy)-4-(5-((((R)-2-((R)-1-(N-((3,4-dimethoxybenzoyl)oxy)formamido)propyl)heptanamido)methyl)carbamoyl)furan-2-yl)benzamido)succinate

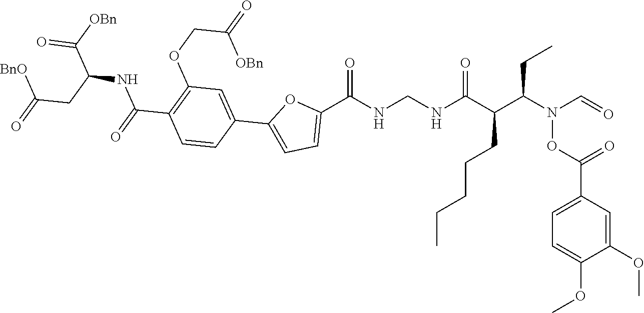 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccc(OC)c(OC)c1]

Methanesulfonyl chloride (0.016 mL, 0.210 mmol) was added to a solution containing 3,4-dimethoxybenzoic acid (0.035 g, 0.191 mmol) and 1-methylimidazole (0.030 mL, 0.382 mmol) in DCM (1.908 mL) at 0° C. After stirring for 30 mins at 0° C., (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (0.178 g, 0.191 mmol), was added and the reaction was stirred at RT overnight. Water was added and the reaction was partitioned between water and DCM. The organic layer was passed through a hydrophobic cartridge and concentrated. Purification by Si (0-100% EtOAc/Hex) afforded the title compound as a colorless oil. (95 mg, 45% yield). MS (m/z) 549.3 (M/2+H)⁺

### Intermediate 54: (S)-dibenzyl 2-(2-(2-(benzyloxy)-2-oxoethoxy)-4-(5-((((R)-2-((R)-1-(N-((2-propylpentanoyl)oxy)formamido)propyl)heptanamido)methyl)carbamoyl)furan-2-yl)benzamido)succinate

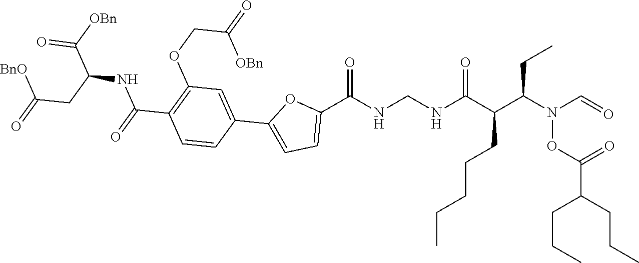 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)C(CCC)CCC]

N-methylmorpholine (70.7 μl, 0.643 mmol) was added to a stirring solution containing 2-propylpentanoic acid (51.5 μl, 0.322 mmol) and HATU (135 mg, 0.356 mmol) in MeCN (1072 μl). This mixture was stirred for 10 min. (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (200 mg, 0.214 mmol) was added in MeCN (1072 μl). The resulting mixture was stirred for 1 h at RT, and was then stirred for 18 h at 50° C. The mixture was concentrated. Purification by Si (0-100% EtOAc/Hex) afforded the title compound as a colorless oil. (157 mg, 69% yield). MS (m/z) 1059.6 (M+H)⁺

### Intermediate 55: (S)-dibenzyl 2-(2-(2-(benzyloxy)-2-oxoethoxy)-4-(5-((((R)-2-((R)-1-(N-((2,4-dimethoxybenzoyl)oxy)formamido)propyl)heptanamido)methyl)carbamoyl)furan-2-yl)benzamido)succinate

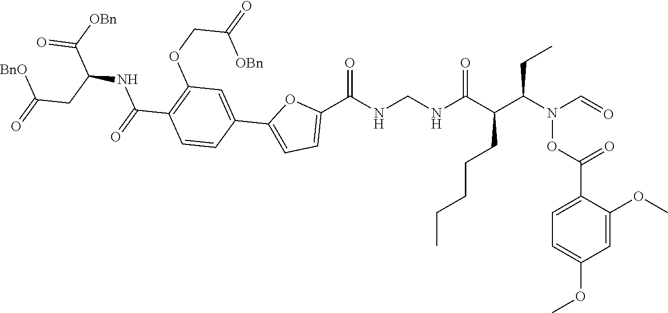 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccc(OC)cc1OC]

Methanesulfonyl chloride (0.011 mL, 0.141 mmol) was added to a solution containing 2,4-dimethoxybenzoic acid (0.023 g, 0.129 mmol) and 1-methylimidazole (0.021 mL, 0.257 mmol) in DCM (1.286 mL) at 0° C. After stirring for 30 mins at 0° C., (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (0.12 g, 0.129 mmol) was added and the reaction was stirred at RT overnight. Water was added and the reaction was partitioned between water and DCM. The organic layer was passed through a hydrophobic cartridge and concentrated. Purification by Si (0-100% EtOAc/Hex) afforded the title compound as a colorless oil. (84 mg, 60% yield).

### Intermediate 56: Dibenzyl (3-ethoxy-5-(5-((((R)-2-((R)-1-(N-((2-ethylbutanoyl)oxy)formamido)propyl)heptanamido)methyl)carbamoyl)furan-2-yl)phenyl)phosphonate

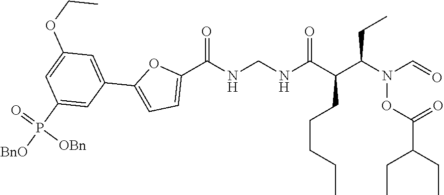 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)C(CC)CC]

2-ethylbutanoyl chloride (28.0 μl, 0.204 mmol) was added to a stirring 0° C. solution containing dibenzyl (3-ethoxy-5-(5-((((R)-2-((R)-1-(N-hydroxyformamido)propyl)heptanamido)methyl)carbamoyl)furan-2-yl)phenyl)phosphonate (100 mg, 0.136 mmol) and TEA (57.0 μl, 0.409 mmol) in MeCN (1363 μl). The mixture was allowed to warm to RT over 1 h and was then concentrated. Purification by Si (0-100% EtOAc/Hex) afforded the title compound as a colorless oil. (114 mg, 100% yield). MS (m/z) 832.6 (M+H)⁺

### Intermediate 57: Dibenzyl (3-ethoxy-5-(5-((((R)-2-((R)-1-(N-((2-fluoro-6-methylbenzoyl)oxy)formamido)propyl)heptanamido)methyl)carbamoyl)furan-2-yl)phenyl)phosphonate

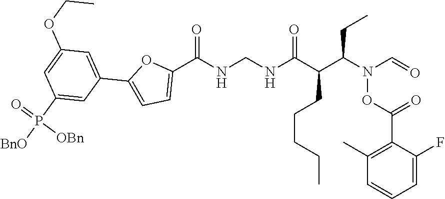 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1c(C)cccc1F]

2-fluoro-6-methylbenzoyl chloride (23.52 mg, 0.136 mmol) was added to a stirring 0° C. solution containing dibenzyl (3-ethoxy-5-(5-((((R)-2-((R)-1-(N-hydroxyformamido)propyl)heptanamido)methyl)carbamoyl)furan-2-yl)phenyl)phosphonate (100 mg, 0.136 mmol) and TEA (57.0 μl, 0.409 mmol) in MeCN (1363 μl). The mixture was allowed to warm to RT over 1 h and concentrated. Purification by Si (0-100% EtOAc/Hex) afforded the title compound as a colorless oil. (109 mg, 91% yield). MS (m/z) 870.5 (M+H)⁺

### Intermediate 58: Dibenzyl (3-ethoxy-5-(5-((((R)-2-((R)-1-(N-((4-methoxybenzoyl)oxy)formamido)propyl)heptanamido)methyl)carbamoyl)furan-2-yl)phenyl)phosphonate

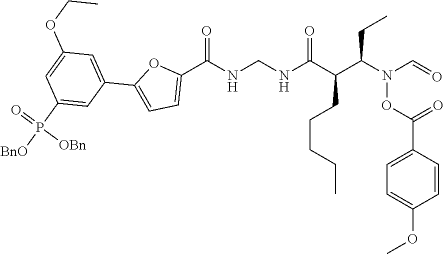 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(OC)cc1]

N-methylmorpholine (44.9 μl, 0.409 mmol) was added to a stirring solution containing dibenzyl (3-ethoxy-5-(5-((((R)-2-((R)-1-(N-hydroxyformamido)propyl)heptanamido)methyl)carbamoyl)furan-2-yl)phenyl)phosphonate (100 mg, 0.136 mmol), HATU (78 mg, 0.204 mmol), and 4-methoxybenzoic acid (31.1 mg, 0.204 mmol) in MeCN (681 μl) and DMF (681 μl). The mixture was stirred at 40° C. for 1 h and was then concentrated. Purification by Si (0-100% EtOAc/Hex) afforded the title compound as a colorless oil. (104 mg, 82% yield). MS (m/z) 868.5 (M+H)⁺

### Intermediate 59: (S)-dibenzyl 2-(2-ethoxy-4-(5-((((R)-2-((R)-1-(N-((2-ethylbutanoyl)oxy)formamido)propyl)heptanamido)methyl)carbamoyl)furan-2-yl)benzamido)succinate

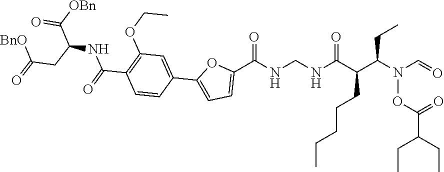 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)C(CC)CC]

2-ethylbutanoyl chloride (25.3 μl, 0.185 mmol) was added to a stirring 0° C. solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (100 mg, 0.123 mmol) and TEA (51.4 μl, 0.369 mmol) in MeCN (1230 μl). The mixture was allowed to warm to RT over 1 h and was then concentrated. Purification by Si (0-100% EtOAc/Hex) afforded the title compound as a colorless oil. (91 mg, 79% yield). MS (m/z) 911.7 (M+H)⁺

### Intermediate 60: (S)-dibenzyl 2-(2-ethoxy-4-(5-((((R)-2-((R)-1-(N-((2-fluoro-6-methylbenzoyl)oxy)formamido)propyl)heptanamido)methyl)carbamoyl)furan-2-yl)benzamido)succinate

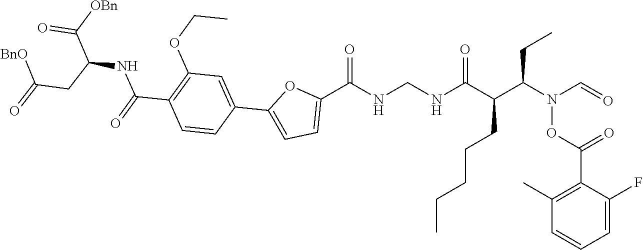 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1c(C)cccc1F]

2-fluoro-6-methylbenzoyl chloride (21.23 mg, 0.123 mmol) was added to a stirring 0° C. solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (100 mg, 0.123 mmol) and TEA (51.4 μl, 0.369 mmol) in MeCN (1230 μl). The mixture was allowed to warm to RT over 1 h and was then concentrated. Purification by Si (0-100% EtOAc/Hex) afforded the title compound as a colorless oil. (77 mg, 65% yield). MS (m/z) 949.7 (M+H)⁺

### Intermediate 61: Dibenzyl (3-ethoxy-5-(5-((((R)-2-((R)-1-(N-((4-(pyrrolidin-1-yl)benzoyl)oxy)formamido)propyl)heptanamido)methyl)carbamoyl)furan-2-yl)phenyl)phosphonate

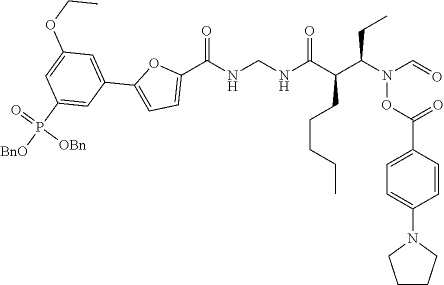 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(N2CCCC2)cc1]

Methanesulfonyl chloride (0.018 mL, 0.225 mmol) was added to a solution containing 4-(pyrrolidin-1-yl)benzoic acid (0.039 g, 0.204 mmol) and 1-methylimidazole (0.033 mL, 0.409 mmol) in DCM (1 mL) at 0° C. After stirring for 30 mins at 0° C., dibenzyl (3-ethoxy-5-(5-((((R)-2-((R)-1-(N-hydroxyformamido)propyl)heptanamido)methyl)carbamoyl)furan-2-yl)phenyl)phosphonate (0.15 g, 0.204 mmol) was added and the reaction was stirred at RT overnight. Water was added and the reaction was partitioned between water and DCM. The organic layer was passed through a hydrophobic cartridge and concentrated. Purification by Si (0-85% EtOAc/Hex) afforded the title compound as a colorless oil. (80 mg, 43% yield). MS (m/z) 907.4 (M+H)⁺

### Intermediate 62: (S)-dibenzyl 2-(2-ethoxy-4-(5-((((R)-2-((R)-1-(N-((4-methoxybenzoyl)oxy)formamido)propyl)heptanamido)methyl)carbamoyl)furan-2-yl)benzamido)succinate

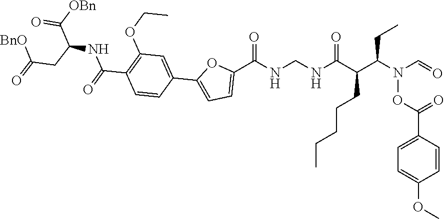 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(OC)cc1]

N-methylmorpholine (40.6 μl, 0.369 mmol) was added to a stirring solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (100 mg, 0.123 mmol), HATU (70.2 mg, 0.185 mmol), and 4-methoxybenzoic acid (28.1 mg, 0.185 mmol) in MeCN (615 μl) and DMF (615 μl). The mixture was stirred at 40° C. for 1 h and was then concentrated. Purification by Si (0-100% EtOAc/Hex) afforded the title compound. (124 mg, 94% yield). MS (m/z) 947.8 (M+H)⁺

### Intermediate 63: (S)-dibenzyl 2-(2-(2-(benzyloxy)-2-oxoethoxy)-4-(5-((((R)-2-((R)-1-(N-((2-isopropyl-4-methoxybenzoyl)oxy)formamido)propyl)heptanamido)methyl)carbamoyl)furan-2-yl)benzamido)succinate

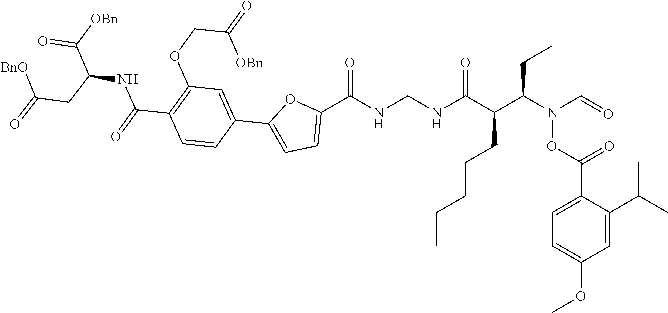 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccc(OC)cc1C(C)C]

oxalyl dichloride (2M in DCM) (335 μl, 0.669 mmol) was slowly added to a stirring solution containing 2-isopropyl-4-methoxybenzoic acid (100 mg, 0.515 mmol) in DMF (3.99 μl, 0.051 mmol) and DCM (1716 μl) at 0° C. The resulting mixture was stirred for 18 h as it warmed to RT. The mixture was then concentrated to dryness to give 2-isopropyl-4-methoxybenzoyl chloride. The crude product was cooled to 0° C., and a solution of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (120 mg, 0.129 mmol) in MeCN (2 mL) was added followed by 4-methylmorpholine (104 mg, 1.030 mmol). The resulting solution was allowed to come to RT over 18 h. The mixture was concentrated. Purification by Si (0-100% EtOAc/Hex) afforded the title compound as a colorless gum. (102 mg, 18% yield). MS (m/z) 1109.7 (M+H)⁺

### Intermediate 64: Methyl 2-isopropyl-4-methoxybenzoate

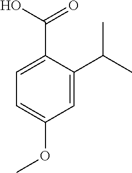

### Step 1: Methyl 4-methoxy-2-(prop-1-en-2-yl)benzoate

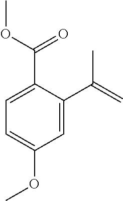

A solution containing methyl 2-bromo-4-methoxybenzoate (1.20 g, 4.90 mmol) and 4,4,5,5-tetramethyl-2-(prop-1-en-2-yl)-1,3,2-dioxaborolane (1.381 mL, 7.34 mmol) was degassed by bubbling N2 through the solution for 10 min. PdCl2(dppf)-CH2Cl2 (0.200 g, 0.245 mmol) was added followed by Na2CO3 (14.69 mL, 14.69 mmol). The resulting mixture was stirred at 50° C. for 3 h. The mixture was diluted with EtOAc and water. The organic phase was washed with brine (2×). Aqueous washes were back extracted using EtOAc (2×). The combined organics were dried over MgSO4, filtered, and concentrated. Purification by Si (0-100% EtOAc/Hex) afforded the title compound as a yellow oil. (619 mg, 61% yield). MS (m/z) 206.9 (M+H)⁺

### Step 2: Methyl 2-isopropyl-4-methoxybenzoate

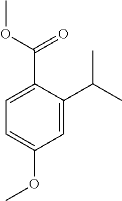

10% Pd—C (0.161 g, 1.515 mmol) was added to a solution containing methyl 4-methoxy-2-(prop-1-en-2-yl)benzoate (0.625 g, 3.03 mmol) in DCM (0.918 mL). MeOH (9.18 mL) was added under N₂. The reaction vial was evacuated and back-filled with N₂ (3×) and was then evacuated and back-filled with H₂ (3×). The mixture was stirred under hydrogen for 3 days. The mixture was evacuated and back-filled with N₂ (3×) and was filtered through a syringe filter washing with MeOH and was concentrated to afford the title compound as an orange oil. (569 mg, 78% yield). MS (m/z) 208.9 (M+H)⁺

### Step 3: Methyl 2-isopropyl-4-methoxybenzoate

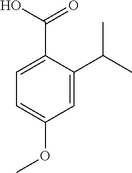

Sodium hydroxide (6M) (2.73 mL, 16.39 mmol) was added to a stirring solution containing methyl 2-isopropyl-4-methoxybenzoate (0.569 g, 2.73 mmol) in MeOH (9.11 mL). The resulting solution was stirred for 18 h at RT. A second portion of sodium hydroxide (6M) (2.73 mL, 16.39 mmol) was added, and the mixture was stirred for 18 h at 50° C. The reaction was concentrated to a viscous solution of water and salts, and HCl (2.73 mL, 16.39 mmol) was added carefully followed by addition of more water. The precipitate was filtered and collected to afford the title compound as a off-white solid. (477 mg, 89% yield). MS (m/z) 194.9 (M+H)⁺

### Intermediate 65: (S)-dibenzyl 2-(2-(2-(benzyloxy)-2-oxoethoxy)-4-(5-((((R)-2-((R)-1-(N-((4-fluoro-2-isopropylbenzoyl)oxy)formamido)propyl)heptanamido)methyl)carbamoyl)furan-2-yl)benzamido)succinate

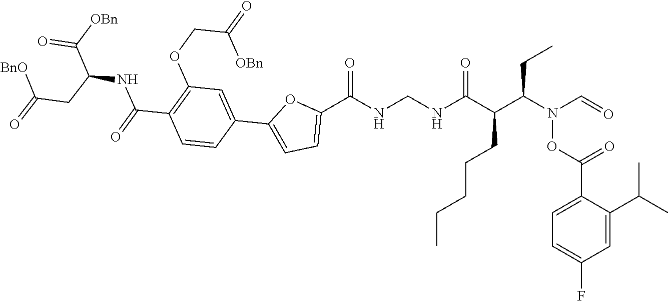 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1ccc(F)cc1C(C)C]

oxalyl dichloride (2 M in DCM) (457 μl, 0.913 mmol) was slowly added to a stirring solution containing 4-fluoro-2-isopropylbenzoic acid (128 mg, 0.703 mmol) and DMF (5.44 μl, 0.070 mmol) in DCM (2342 μl) at 0° C. The resulting mixture was stirred for 18 h as it warmed to RT. The mixture was then concentrated to dryness to give 4-fluoro-2-isopropylbenzoyl chloride. The acid chloride was cooled to 0° C., and a solution of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (164 mg, 0.176 mmol) in MeCN (2 mL) was added followed by 4-methylmorpholine (142 mg, 1.405 mmol). The resulting solution was allowed to come to RT over 18 h. The mixture was concentrated. Purification by Si (0-100% EtOAc/Hex) afforded the title compound as a colorless gum. (102 mg, 18% yield). MS (m/z) 1097.6 (M+H)⁺

### Intermediate 66: 4-fluoro-2-isopropylbenzoic Acid

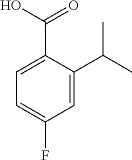

### Step 1: Methyl 4-fluoro-2-(prop-1-en-2-yl)benzoate

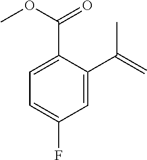

A solution containing methyl 2-bromo-6-fluorobenzoate (1.00 g, 4.29 mmol) and 4,4,5,5-tetramethyl-2-(prop-1-en-2-yl)-1,3,2-dioxaborolane (1.210 mL, 6.44 mmol) was degassed by bubbling N2 through the solution for 10 min. PdCl2(dppf)-CH2Cl2 (0.175 g, 0.215 mmol) was added followed by Na₂CO₃ (12.87 mL, 12.87 mmol). The resulting mixture was stirred at 50° C. for 3 h. The mixture was diluted with EtOAc and water. The mixture was washed with brine (2×). Aqueous washes were back extracted using EtOAc (2×). The combined organic phase was dried over MgSO4, filtered, and concentrated. Purification by Si (0-100% EtOAc/Hex) afforded the title compound as a clear oil. (625 mg, 75% yield). MS (m/z) 194.9 (M+H)⁺

### Step 2: Methyl 4-fluoro-2-isopropylbenzoate

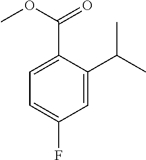

Pd—C (0.123 g, 1.156 mmol) was added to a solution containing methyl 2-fluoro-6-(prop-1-en-2-yl)benzoate (0.449 g, 2.312 mmol) in DCM (0.701 mL). MeOH (7.01 mL) was added under N₂. The reaction vial was evacuated and back-filled with N₂ (3×) and was then evacuated and back-filled with H₂ (3×). The mixture was stirred under H₂ for 18 h. The mixture was evacuated and back-filled with N₂ (3×) and was filtered through a syringe filter washing with MeOH and was concentrated to afford the title compound as a yellow oil. (195 mg, 32% yield). MS (m/z) 196.9 (M+H)⁺

### Step 3: 4-fluoro-2-Isopropylbenzoic Acid

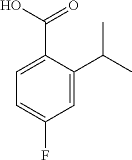

Sodium hydroxide (2981 μl, 5.96 mmol) was added to a stirring solution containing methyl 4-fluoro-2-isopropylbenzoate (195 mg, 0.994 mmol) in MeOH (3313 μl). The resulting solution was stirred for 18 h at RT. HCl (994 μl, 5.96 mmol) was added, and the mixture was concentrated to dryness. Product is an oil, so EtOAc was added, and the organics were washed with water (2×) and brine (1×). The organics phase was dried over MgSO₄, filtered, and concentrated to afford the title compound. (128 mg, 67% yield). MS (m/z) 182.9 (M+H)⁺

### Intermediate 67: (S)-dibenzyl 2-(2-ethoxy-4-(5-((((R)-2-((R)-1-(N-((4-methoxy-2-methylbenzoyl)oxy)formamido)propyl)heptanamido)methyl)carbamoyl)furan-2-yl)benzamido)succinate

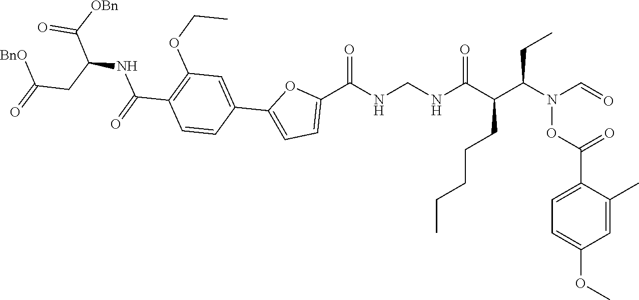 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(OC)cc1C]

HATU (0.103 g, 0.271 mmol) was added to a solution containing 4-methoxy-2-methylbenzoic acid (0.041 g, 0.246 mmol) and DIPEA (0.129 mL, 0.738 mmol) in MeCN (1.5 mL). After stirring for 30 mins, (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.2 g, 0.246 mmol) was added and the reaction was stirred at RT overnight. Water was added and the reaction was extracted with EtOAc. The organic layer was dried over Na₂SO₄ and concentrated. Purification by Si (0-80% EtOAc/Hex) afforded the title compound as a thin film. (100 mg, 42% yield). MS (m/z) 961.4 (M+H)⁺

### Intermediate 68: Dibenzyl (3-ethoxy-5-(5-(((4R,5R)-5-ethyl-6-formyl-10,10-dimethyl-3,8-dioxo-4-pentyl-7-oxa-2,6,9-triazaundecyl)carbamoyl)furan-2-yl)phenyl)phosphonate

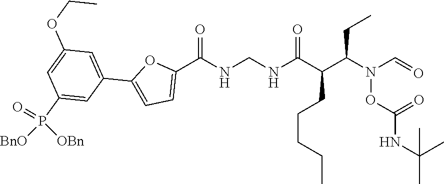 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)NC(C)(C)C]

Dibenzyl (3-ethoxy-5-(5-((((R)-2-((R)-1-(N-hydroxyformamido)propyl)heptanamido)methyl)carbamoyl)furan-2-yl)phenyl)phosphonate (0.1 g, 0.136 mmol) in DCM (1.4 mL) was added to a solution containing 2-isocyanato-2-methylpropane (0.078 mL, 0.681 mmol) in DCM (1.363 mL) at 0° C. TEA (0.095 mL, 0.681 mmol) was then added and the reaction was warmed to RT over 18 h. The reaction was concentrated. Purification by Si (20-100% EtOAc/Hex) afforded the title compound as a thin film. (90 mg, 79% yield). MS (m/z) 833.3 (M+H)⁺

### Intermediate 69: Dibenzyl (3-ethoxy-5-(5-(((4R,5R)-5-ethyl-6-formyl-10-methyl-3,8-dioxo-4-pentyl-10-phenyl-7-oxa-2,6,9-triazaundecyl)carbamoyl)furan-2-yl)phenyl)phosphonate

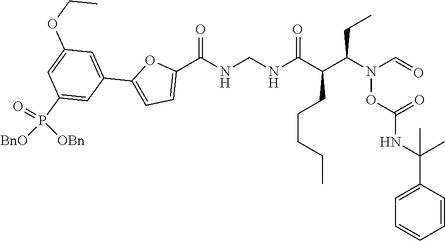 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)NC(C)(C)c1ccccc1]

Dibenzyl (3-ethoxy-5-(5-((((R)-2-((R)-1-(N-hydroxyformamido)propyl)heptanamido)methyl)carbamoyl)furan-2-yl)phenyl)phosphonate (0.1 g, 0.136 mmol) in DCM (1.4 mL) was added to a solution containing (2-isocyanatopropan-2-yl)benzene (0.110 g, 0.681 mmol) in DCM (1.363 mL) at 0° C. TEA (0.095 mL, 0.681 mmol) was then added and the reaction was warmed to RT over 18 h. The reaction was concentrated. Purification by Si (20-100% EtOAc/Hex) afforded the title compound as a thin film. (80 mg, 66% yield). MS (m/z) 895.3 (M+H)⁺

### Intermediate 70: Dibenzyl (3-ethoxy-5-(5-((((R)-2-((R)-1-(N-((4-morpholinobenzoyl)oxy)formamido)propyl)heptanamido)methyl)carbamoyl)furan-2-yl)phenyl)phosphonate

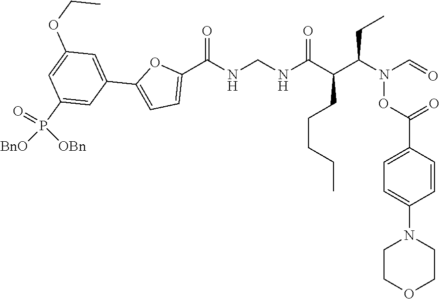 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(N2CCOCC2)cc1]

Methanesulfonyl chloride (0.023 mL, 0.300 mmol) was added to a solution containing 4-morpholinobenzoic acid (0.056 g, 0.273 mmol) and 1-methylimidazole (0.043 mL, 0.545 mmol) in DCM (1.5 mL) at 0° C. After stirring for 15 mins at 0° C. and then at RT for 15 mins, dibenzyl (3-ethoxy-5-(5-((((R)-2-((R)-1-(N-hydroxyformamido)propyl)heptanamido)methyl)carbamoyl)furan-2-yl)phenyl)phosphonate (0.2 g, 0.273 mmol) was added and the reaction was stirred at RT overnight. Water was added and the reaction was partitioned between water and DCM. The organic phase was passed through a hydrophobic cartridge and concentrated. Purification by Si (0-80% EtOAc/Hex) afforded the title compound as a colorless solid. (130 mg, 52% yield). MS (m/z) 923.4 (M+H)⁺

### Intermediate 71: Dibenzyl (3-(5-((((R)-2-((R)-1-(N-((4-(1H-pyrrol-1-yl)benzoyl)oxy)formamido)propyl)heptanamido)methyl)carbamoyl)furan-2-yl)-5-ethoxyphenyl)phosphonate

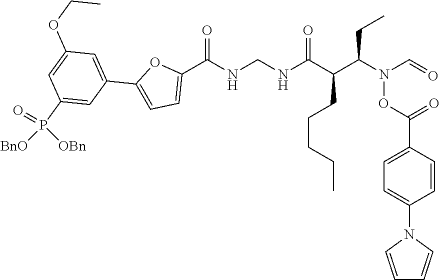 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(-n2cccc2)cc1]

Methanesulfonyl chloride (0.023 mL, 0.300 mmol) was added to a solution containing 4-(1H-pyrrol-1-yl)benzoic acid (0.051 g, 0.273 mmol) and 1-methylimidazole (0.043 mL, 0.545 mmol) in DCM (1.5 mL) at 0° C. After stirring for 30 mins at 0° C., dibenzyl (3-ethoxy-5-(5-((((R)-2-((R)-1-(N-hydroxyformamido)propyl)heptanamido)methyl)carbamoyl)furan-2-yl)phenyl)phosphonate (0.2 g, 0.273 mmol) was added and the reaction was stirred at RT overnight. Water was added and the reaction was partitioned between water and DCM. The organic layer was passed through a hydrophobic cartridge and concentrated. Purification by Si (0-80% EtOAc/Hex) afforded the title compound as a colorless solid. (160 mg, 65% yield). MS (m/z) 903.4 (M+H)⁺

### Intermediate 72: Dibenzyl (3-ethoxy-5-(5-((((R)-2-((R)-1-(N-((4-(2-oxopyrrolidin-1-yl)benzoyl)oxy)formamido)propyl)heptanamido)methyl)carbamoyl)furan-2-yl)phenyl)phosphonate

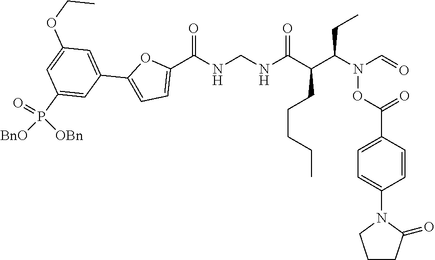 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(N2CCCC2=O)cc1]

Methanesulfonyl chloride (0.023 mL, 0.300 mmol) was added to a solution containing 4-(2-oxopyrrolidin-1-yl)benzoic acid (0.056 g, 0.273 mmol) and 1-methylimidazole (0.043 mL, 0.545 mmol) in DCM (1.5 mL) at 0° C. The reaction was stirred at 0° C. for 10 mins and then at RT for 20 mins. The reaction was cooled down in an ice bath again and dibenzyl (3-ethoxy-5-(5-((((R)-2-((R)-1-(N-hydroxyformamido)propyl)heptanamido)methyl)carbamoyl)furan-2-yl)phenyl)phosphonate (0.2 g, 0.273 mmol) was added and the reaction was stirred at RT overnight. Water was added and the reaction was partitioned between water and DCM. The organic phase was passed through a hydrophobic cartridge and concentrated. Purification by Si (0-80% EtOAc/Hex) afforded the title compound as a colorless solid. (134 mg, 53% yield). MS (m/z) 921.4 (M+H)⁺

### Intermediate 73: Dibenzyl (3-ethoxy-5-(5-((((R)-2-((R)-1-(N-((4-methyl-3,4-dihydro-2H-benzo[b][1,4]oxazine-7-carbonyl)oxy)formamido)propyl)heptanamido)methyl)carbamoyl)furan-2-yl)phenyl)phosphonate

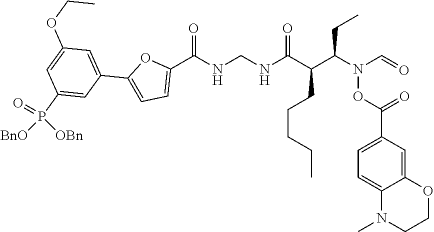 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc2c(c1)OCCN2C]

Methanesulfonyl chloride (0.023 mL, 0.300 mmol) was added to a solution of 4-methyl-3,4-dihydro-2H-benzo[b][1,4]oxazine-7-carboxylic acid (0.053 g, 0.273 mmol) and 1-methylimidazole (0.043 mL, 0.545 mmol) in DCM (1.5 mL) at 0° C. The reaction was stirred at 0° C. for 10 mins and then at RT for 20 mins. The reaction was cooled down in an ice bath again and, dibenzyl (3-ethoxy-5-(5-((((R)-2-((R)-1-(N-hydroxyformamido)propyl)heptanamido)methyl)carbamoyl)furan-2-yl)phenyl)phosphonate (0.2 g, 0.273 mmol) was added and the reaction was stirred at RT overnight. Water was added and the reaction was partitioned between water and DCM. The organic layer was passed through a hydrophobic cartridge and concentrated. Purification by Si (0-100% EtOAc/Hex) afforded the title compound as a colorless solid. (116 mg, 47% yield). MS (m/z) 909.4 (M+H)⁺

### Intermediate 74: (2-((4R,5R)-10-(5-(3-(bis(benzyloxy)phosphoryl)-5-ethoxyphenyl)furan-2-yl)-4-ethyl-3-formyl-6,10-dioxo-5-pentyl-2-oxa-3,7,9-triazadecan-1-oyl)-3-methylphenoxy)methyl Dihydrogen Phosphate

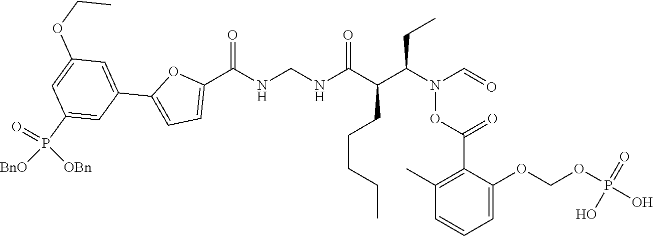 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1c(C)cccc1OCOP(=O)(O)O]

### Step 1: (2-((4R,5R)-10-(5-(3-(bis(benzyloxy)phosphoryl)-5-ethoxyphenyl)furan-2-yl)-4-ethyl-3-formyl-6,10-dioxo-5-pentyl-2-oxa-3,7,9-triazadecan-1-oyl)-3-methylphenoxy)methyl di-tert-butyl Phosphate

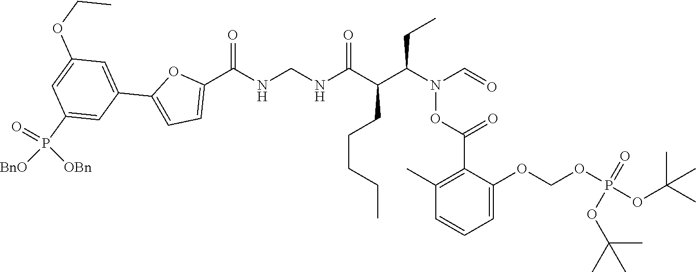 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1c(C)cccc1OCOP(=O)(OC(C)(C)C)OC(C)(C)C]

2-(((di-tert-butoxyphosphoryl)oxy)methoxy)-6-methylbenzoic acid (0.18 g, 0.433 mmol) was added to a stirring solution of HATU (0.181 g, 0.476 mmol), dibenzyl (3-ethoxy-5-(5-((((R)-2-((R)-1-(N-hydroxyformamido)propyl)heptanamido)methyl)carbamoyl)furan-2-yl)phenyl)phosphonate (0.25 g, 0.341 mmol) and TEA (0.151 mL, 1.082 mmol) in MeCN (4.33 mL). The reaction was stirred at room temp for 12 h and concentrated. Purification by Si (0-100% EtOAc/Hex) afforded the title compound as a colorless solid. (190 mg, 40% yield). MS (m/z) 1090.6 (M+H)⁺

### Step 2: (2-((4R,5R)-10-(5-(3-(bis(benzyloxy)phosphoryl)-5-ethoxyphenyl)furan-2-yl)-4-ethyl-3-formyl-6,10-dioxo-5-pentyl-2-oxa-3,7,9-triazadecan-1-oyl)-3-methylphenoxy)methyl Dihydrogen Phosphate

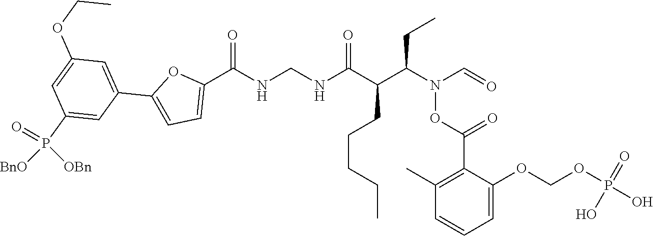 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1c(C)cccc1OCOP(=O)(O)O]

(2-((4R,5R)-10-(5-(3-(bis(benzyloxy)phosphoryl)-5-ethoxyphenyl)furan-2-yl)-4-ethyl-3-formyl-6,10-dioxo-5-pentyl-2-oxa-3,7,9-triazadecan-1-oyl)-3-methylphenoxy)methyl di-tert-butyl phosphate (190 mg, 0.17 mmol) was taken up in DCM (20 mL) and treated with TFA (2 mL). The reaction was stirred for 18 h and concentrated to afford the title compound as a clear oil. (150 mg, 88% yield). MS (m/z) 978.2 (M+H)⁺

### Intermediate 75: 2-(((di-tert-butoxyphosphoryl)oxy)methoxy)-6-methylbenzoic Acid

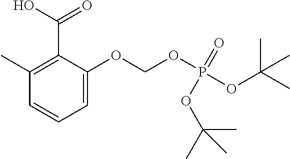 [SMILES: Cc1cccc(OCOP(=O)(OC(C)(C)C)OC(C)(C)C)c1C(=O)O]

### Step 1: Allyl 2-hydroxy-6-methylbenzoate

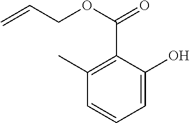

To a solution containing 2-hydroxy-6-methylbenzoic acid (1.82 g, 11.72 mmol) MeCN (58.6 mL) was added cesium carbonate (3.82 g, 11.72 mmol), followed by addition of 3-bromoprop-1-ene (1.116 mL, 12.90 mmol). The reaction mixture was stirred at 50° C. overnight and concentrated. Purification by Si (0-100% EtOAc/Hex) afforded the title compound as a colorless solid. (1250 mg, 56% yield). MS (m/z) 192.9 (M+H)⁺

### Step 2: Allyl 2-(((di-tert-butoxyphosphoryl)oxy)methoxy)-6-methylbenzoate

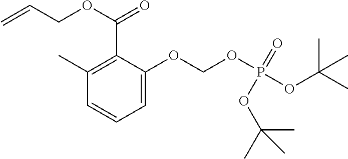 [SMILES: C=CCOC(=O)c1c(C)cccc1OCOP(=O)(OC(C)(C)C)OC(C)(C)C]

To a solution containing allyl 2-hydroxy-6-methylbenzoate (0.88 g, 4.58 mmol) and tetrabutylammonium iodide (0.101 g, 0.275 mmol) in DMF (7 mL) was added sodium hydride (0.275 g, 6.87 mmol). The mixture was stirred for 15 mins and then a solution of di-tert-butyl (chloromethyl) phosphate (1.184 g, 4.58 mmol) in DMF (5 mL) was added. The reaction mixture was heated to 50° C. overnight and concentrated. Purification by Si (0-100% EtOAc/Hex) afforded the title compound as a colorless solid. (1.41 g, 74% yield). MS (m/z) 415.1 (M+H)⁺

### Step 3: 2-(((di-tert-butoxyphosphoryl)oxy)methoxy)-6-methylbenzoic Acid

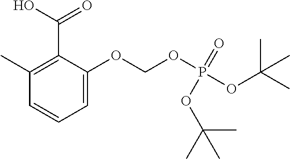 [SMILES: Cc1cccc(OCOP(=O)(OC(C)(C)C)OC(C)(C)C)c1C(=O)O]

To a solution containing allyl 2-(((di-tert-butoxyphosphoryl)oxy)methoxy)-6-methylbenzoate (0.2 g, 0.483 mmol) DCM (3 mL) was added 1,3-dimethylpyrimidine-2,4,6(1H,3H,5H)-trione (0.226 g, 1.448 mmol) and Pd(Ph3p)4 (0.112 g, 0.097 mmol). The reaction was stirred at RT overnight. The reaction mixture was filtered through celite and concentrated. Purification by Si (0-100% EtOAc/Hex) afforded the title compound as a orange oil. (100 mg, 55% yield). MS (m/z) 397.1 (M+23)+

### Intermediate 76: Dibenzyl (3-ethoxy-5-(5-((((R)-2-((R)-1-(N-((3-fluoro-4-(pyrrolidin-1-yl)benzoyl)oxy)formamido)propyl)heptanamido)methyl)carbamoyl)furan-2-yl)phenyl)phosphonate

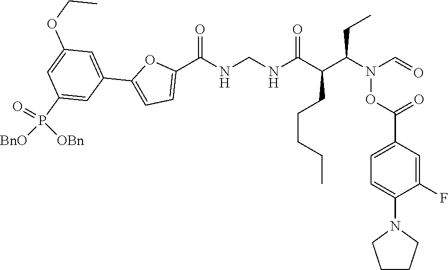 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(N2CCCC2)c(F)c1]

HATU (0.093 g, 0.245 mmol) was added to a solution containing 3-fluoro-4-(pyrrolidin-1-yl)benzoic acid (0.047 g, 0.225 mmol) and DIPEA (0.107 mL, 0.613 mmol) in MeCN (1.363 mL). After stirring for 30 mins, dibenzyl (3-ethoxy-5-(5-((((R)-2-((R)-1-(N-hydroxyformamido)propyl)heptanamido)methyl)carbamoyl)furan-2-yl)phenyl)phosphonate (0.15 g, 0.204 mmol) was added and the reaction was stirred at RT overnight. Water was added and the reaction was partitioned between water and EtOAc. The organic layer was dried over Na₂SO₄ and concentrated. Purification by Si (0-80% EtOAc/Hex) afforded the title compound as a colorless solid. (160 mg, 85% yield). MS (m/z) 926.4 (M+H)⁺

### Intermediate 77: Dibenzyl (3-ethoxy-5-(5-((((R)-2-((R)-1-(N-((3-(pyrrolidin-1-yl)benzoyl)oxy)formamido)propyl)heptanamido)methyl)carbamoyl)furan-2-yl)phenyl)phosphonate

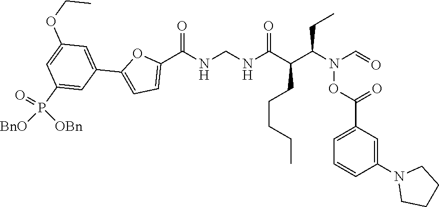 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1cccc(N2CCCC2)c1]

HATU (0.093 g, 0.245 mmol) was added to a solution of 3-(pyrrolidin-1-yl)benzoic acid (0.043 g, 0.225 mmol) and DIPEA (0.107 mL, 0.613 mmol) in MeCN (1.5 mL). After stirring for 30 mins, dibenzyl (3-ethoxy-5-(5-((((R)-2-((R)-1-(N-hydroxyformamido)propyl)heptanamido)methyl)carbamoyl)furan-2-yl)phenyl)phosphonate (0.15 g, 0.204 mmol) was added and the reaction was stirred at RT overnight. Water was added and the reaction was partitioned between water and EtOAc. The organic layer was dried over Na₂SO₄ and concentrated. Purification by Si (0-80% EtOAc/Hex) afforded the title compound as a colorless solid. (152 mg, 82% yield). MS (m/z) 907.4 (M+H)⁺

### Intermediate 78: Dibenzyl (3-ethoxy-5-(5-((((R)-2-((R)-1-(N-((2-methyl-4-(morpholinomethyl)benzoyl)oxy)formamido)propyl)heptanamido)methyl)carbamoyl)furan-2-yl)phenyl)phosphonate

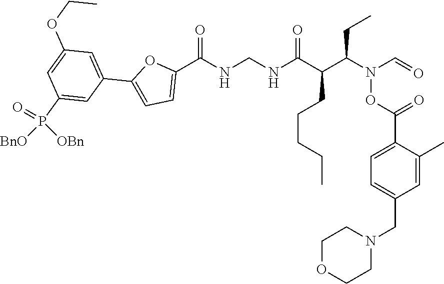 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(CN2CCOCC2)cc1C]

N-methylmorpholine (44.9 μl, 0.409 mmol) was added to a stirring solution containing dibenzyl (3-ethoxy-5-(5-((((R)-2-((R)-1-(N-hydroxyformamido)propyl)heptanamido)methyl)carbamoyl)furan-2-yl)phenyl)phosphonate (100 mg, 0.136 mmol), HATU (78 mg, 0.204 mmol), and 2-methyl-4-(morpholinomethyl)benzoic acid hydrochloride (37.0 mg, 0.136 mmol) in MeCN (681 μl) and DMF (681 μl). The mixture was stirred at 40° C. for 18 h and was then concentrated. Purification by Si (0-100% EtOAc/Hex and then 5% MeOH in EtOAc) afforded the title compound as a colorless solid. (118 mg, 91% yield). MS (m/z) 951.4 (M+H)⁺

### Intermediate 79: Dibenzyl (3-ethoxy-5-(5-(((4R,5R)-5-ethyl-6-formyl-10-methyl-3,8-dioxo-4-pentyl-7-oxa-2,6,9-triazaundecyl)carbamoyl)furan-2-yl)phenyl)phosphonate

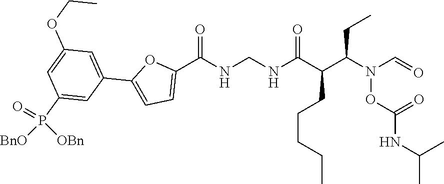 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)NC(C)C]

2-isocyanatopropane (20.09 μl, 0.204 mmol) was added to a stirring solution of dibenzyl (3-ethoxy-5-(5-((((R)-2-((R)-1-(N-hydroxyformamido)propyl)heptanamido)methyl)carbamoyl)furan-2-yl)phenyl)phosphonate (100 mg, 0.136 mmol) and TEA (57.0 μl, 0.409 mmol) in MeCN (681 μl). The mixture was stirred at RT for 1 h and was then concentrated. Purification by Si (0-100% EtOAc/Hex) afforded the title compound as a colorless solid. (98 mg, 88% yield). MS (m/z) 819.5 (M+H)⁺

### Intermediate 80: (S)-dibenzyl 2-(2-ethoxy-4-(5-((((R)-2-((R)-1-(N-((2-isopropyl-4-methoxybenzoyl)oxy)formamido)propyl)heptanamido)methyl)carbamoyl)furan-2-yl)benzamido)succinate

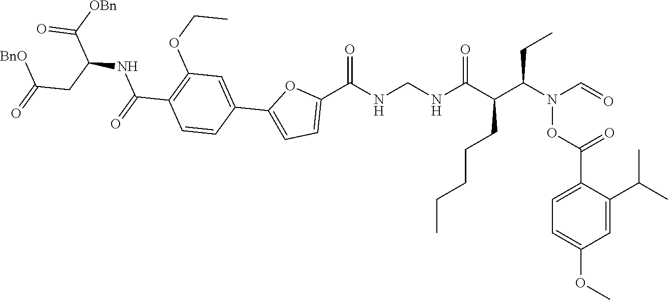 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(OC)cc1C(C)C]

Oxalyl dichloride (2 M in DCM) (335 μl, 0.669 mmol) was slowly added to a stirring solution containing 2-isopropyl-4-methoxybenzoic acid (100 mg, 0.515 mmol) and DMF (3.99 μl, 0.051 mmol) in DCM (1716 μl). The resulting mixture was stirred for 18 h as it warmed to RT. The mixture was then concentrated to dryness to give 2-isopropyl-4-methoxybenzoyl chloride. The crude product was cooled to 0° C., and a solution of (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (105 mg, 0.129 mmol) in MeCN (2 mL) was added followed by TEA (215 μl, 1.545 mmol). The resulting solution was allowed to come to RT over 18 h. The mixture was concentrated. Purification by Si (0-100% EtOAc/Hex) afforded the title compound as a colorless solid. (78 mg, 12% yield). MS (m/z) 989.4 (M+H)⁺

### Intermediate 81: (S)-dibenzyl 2-(2-ethoxy-4-(5-((((R)-2-((R)-1-(N-((4-fluoro-2-isopropylbenzoyl)oxy)formamido)propyl)heptanamido)methyl)carbamoyl)furan-2-yl)benzamido)succinate

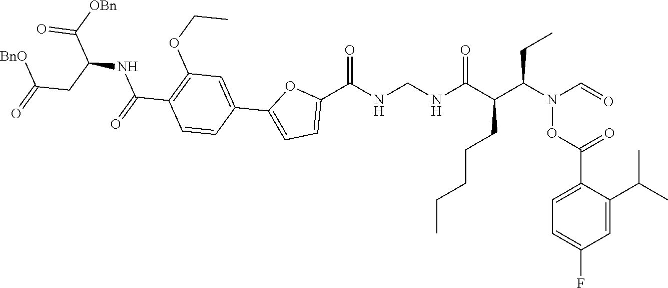 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(F)cc1C(C)C]

Oxalyl dichloride (2 M in DCM) (357 μl, 0.714 mmol) was slowly added to a stirring solution containing 4-fluoro-2-isopropylbenzoic acid (100 mg, 0.549 mmol) and DMF (4.25 μl, 0.055 mmol) in DCM (1830 μl). The resulting mixture was stirred for 18 h as it warmed to RT. The mixture was then concentrated to dryness to give 4-fluoro-2-isopropylbenzoyl chloride. The crude product was cooled to 0° C., and a solution of (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (112 mg, 0.137 mmol) in MeCN (2 mL) was added followed by TEA (230 μl, 1.647 mmol). The resulting solution was allowed to come to RT over 18 h and concentrated. Purification by Si (0-100% EtOAc/Hex) afforded the title compound as a colorless solid. (52 mg, 10% yield). MS (m/z) 977.7 (M+H)⁺

### Intermediate 82: Dibenzyl (3-ethoxy-5-(5-((((R)-2-((R)-1-(N-((4-fluoro-2-isopropylbenzoyl)oxy)formamido)propyl)heptanamido)methyl)carbamoyl)furan-2-yl)phenyl)phosphonate

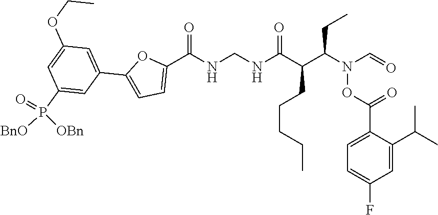 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(F)cc1C(C)C]

Oxalyl dichloride (2 M in DCM) (357 μl, 0.714 mmol) was slowly added to a stirring solution containing 4-fluoro-2-isopropylbenzoic acid (100 mg, 0.549 mmol) and DMF (4.25 μl, 0.055 mmol) in DCM (1830 μl). The resulting mixture was stirred for 18 h as it warmed to RT. The mixture was then concentrated to dryness to give 4-fluoro-2-isopropylbenzoyl chloride. The crude product was cooled to 0° C., and a solution of dibenzyl (3-ethoxy-5-(5-((((R)-2-((R)-1-(N-hydroxyformamido)propyl)heptanamido)methyl)carbamoyl)furan-2-yl)phenyl)phosphonate (101 mg, 0.137 mmol) in MeCN (2 mL) was added followed by TEA (230 μl, 1.647 mmol). The resulting solution was allowed to come to RT over 18 h and concentrated. Purification by Si (0-100% EtOAc/Hex) afforded the title compound as a colorless solid. (103 mg, 21% yield). MS (m/z) 898.5 (M+H)⁺

### Intermediate 83: Dibenzyl (3-ethoxy-5-(5-((((R)-2-((R)-1-(N-(nonanoyloxy)formamido)propyl)heptanamido)methyl)carbamoyl)furan-2-yl)phenyl)phosphonate

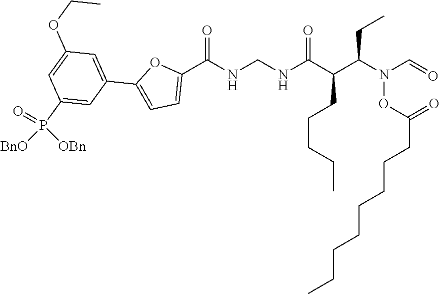 [SMILES: CCCCCCCCC(=O)ON(C=O)[C@H](CC)[C@@H](CCCCC)C(=O)NCNC(=O)c1ccc(-c2cc(OCC)cc(P(=O)(OCc3ccccc3)OCc3ccccc3)c2)o1]

N-methylmorpholine (44.9 μl, 0.409 mmol) was added to a stirring solution containing dibenzyl (3-ethoxy-5-(5-((((R)-2-((R)-1-(N-hydroxyformamido)propyl)heptanamido)methyl)carbamoyl)furan-2-yl)phenyl)phosphonate (100 mg, 0.136 mmol), HATU (78 mg, 0.204 mmol), and nonanoic acid (35.7 μl, 0.204 mmol) in MeCN (681 μl) and DMF (681 μl). The mixture was stirred at 40° C. for 18 h and was then concentrated. Purification by Si (0-100% EtOAc/Hex) afforded the title compound as a colorless solid. (132 mg, 80% yield). MS (m/z) 874.3 (M+H)⁺

### Intermediate 84: (S)-dibenzyl 2-(2-ethoxy-4-(5-((((R)-2-((R)-1-(N-((4-(morpholinomethyl)benzoyl)oxy)formamido)propyl)heptanamido)methyl)carbamoyl)furan-2-yl)benzamido)succinate

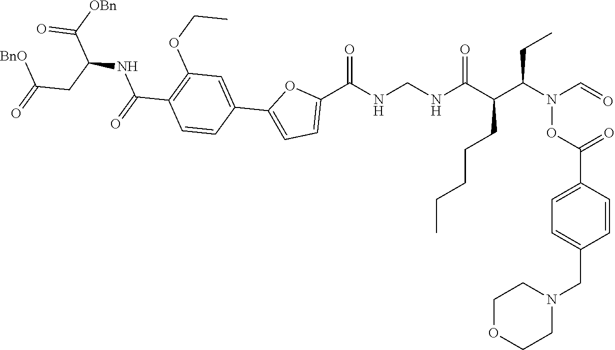 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(CN2CCOCC2)cc1]

N-methylmorpholine (32.5 μl, 0.295 mmol) was added to a stirring solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (80 mg, 0.098 mmol), HATU (56.1 mg, 0.148 mmol), and 4-(morpholinomethyl)benzoic acid hydrochloride (25.4 mg, 0.1 mmol) in MeCN (492 μl) and DMF (492 μl). The mixture was stirred at 40° C. for 18 h and was then concentrated. Purification by Si (0-100% EtOAc/Hex) afforded the title compound as a colorless solid. (115 mg, 93% yield). MS (m/z) 1016.4 (M+H)⁺

### Intermediate 85: 4-(morpholinomethyl)benzoic Acid, Hydrochloride

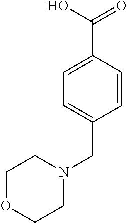 [SMILES: O=C(O)c1ccc(CN2CCOCC2)cc1]

To a solution containing morpholine (5.65 mL, 65.5 mmol) in EtOH (50 mL) was added potassium iodide (1.449 g, 8.73 mmol), potassium carbonate (15.08 g, 109 mmol) followed by methyl 4-(bromomethyl)benzoate (10 g, 43.7 mmol). The reaction was stirred at RT for 18 h. Reaction mixture was diluted with water and extracted with EtOAc, dried over MgSO4, filtered and concentrated. The resulting residue was taken up in sodium hydroxide (65.5 mL, 131 mmol) and stirred for 18 h. hydrochloric acid (43.7 mL, 262 mmol) was added and the reaction was chilled to 0° C. and stirred for 1 hr. The resulting precipitate was filtered and washed with hexanes to afford the title compound as a colorless solid. (10.1 g, 89% yield). MS (m/z) 222.0 (M+H)⁺

### Intermediate 86: (S)-dibenzyl 2-(2-ethoxy-4-(5-((((R)-2-((R)-1-(N-((2-methyl-4-(morpholinomethyl)benzoyl)oxy)formamido)propyl)heptanamido)methyl)carbamoyl)furan-2-yl)benzamido)succinate

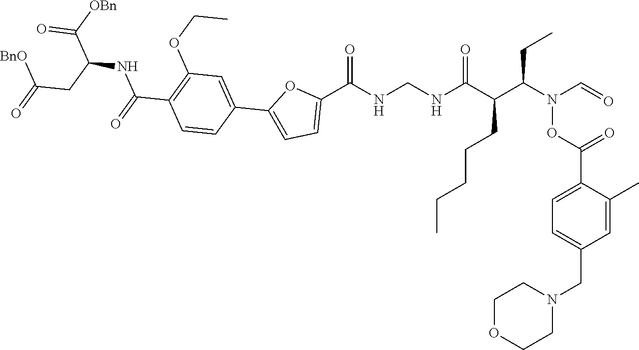 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(CN2CCOCC2)cc1C]

N-methylmorpholine (32.5 μl, 0.295 mmol) was added to a stirring solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (80 mg, 0.098 mmol), HATU (56.1 mg, 0.148 mmol), and 2-methyl-4-(morpholinomethyl)benzoic acid hydrochloride (26.7 mg, 0.1 mmol) in MeCN (492 μl) and DMF (492 μl). The mixture was stirred at 40° C. for 18 h and was then concentrated. Purification by Si (0-100% EtOAc/Hex and then 5% MeOH in EtOAc) afforded the title compound as a colorless solid. (107 mg, 67% yield). MS (m/z) 1030.6 (M+H)⁺

### Intermediate 87: (S)-dibenzyl 2-(2-ethoxy-4-(5-(((4R,5R)-5-ethyl-6-formyl-10-methyl-3,8-dioxo-4-pentyl-7-oxa-2,6,9-triazaundecyl)carbamoyl)furan-2-yl)benzamido)succinate

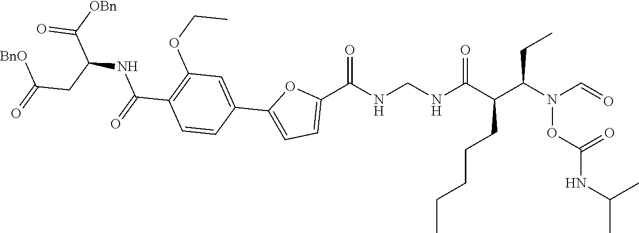 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)NC(C)C]

2-isocyanatopropane (14.51 μl, 0.148 mmol) was added to a stirring solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (80 mg, 0.098 mmol) and TEA (41.2 μl, 0.295 mmol) in MeCN (984 μl). The mixture was stirred at RT for 1 h and concentrated. Purification by Si (0-100% EtOAc/Hex and then 5% MeOH in EtOAc) afforded the title compound as a colorless solid. (60 mg, 66% yield). MS (m/z) 898.5 (M+H)⁺

### Intermediate 88: Dibenzyl (3-ethoxy-5-(5-((((R)-2-((R)-1-(N-((4-(morpholinomethyl)benzoyl)oxy)formamido)propyl)heptanamido)methyl)carbamoyl)furan-2-yl)phenyl)phosphonate

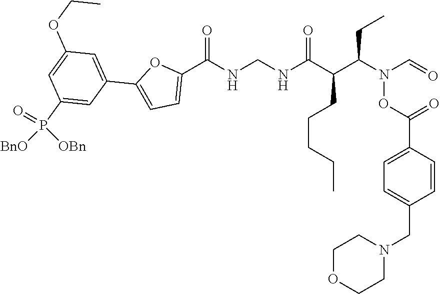 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(CN2CCOCC2)cc1]

N-methylmorpholine (44.9 μl, 0.409 mmol) was added to a stirring solution containing dibenzyl (3-ethoxy-5-(5-((((R)-2-((R)-1-(N-hydroxyformamido)propyl)heptanamido)methyl)carbamoyl)furan-2-yl)phenyl)phosphonate (100 mg, 0.136 mmol), HATU (78 mg, 0.204 mmol), and 4-(morpholinomethyl)benzoic acid hydrochloride (35.1 mg, 0.136 mmol) in MeCN (681 μl) and DMF (681 μl). The mixture was stirred at 40° C. for 18 h and concentrated. Purification by Si (0-100% EtOAc/Hex and then 5% MeOH in EtOAc) afforded the title compound as a colorless solid. (132 mg, 92% yield). MS (m/z) 937.4 (M+H)⁺

### Intermediate 89: (S)-dibenzyl 2-(2-ethoxy-4-(5-(((4R,5R)-5-ethyl-6-formyl-10,10-dimethyl-3,8-dioxo-4-pentyl-7-oxa-2,6,9-triazaundecyl)carbamoyl)furan-2-yl)benzamido)succinate

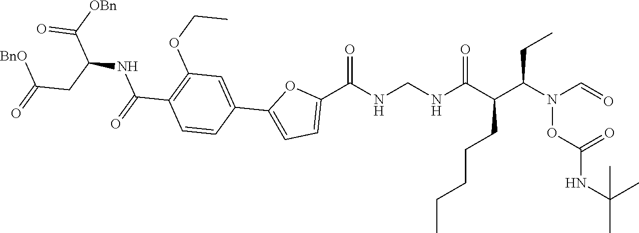 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)NC(C)(C)C]

(S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.1 g, 0.123 mmol) was added to a solution of 2-isocyanato-2-methylpropane (0.070 mL, 0.615 mmol) in DCM (1.230 mL) at 0° C. The reaction was stirred at RT overnight and concentrated. Purification by Si (20-100% EtOAc/Hex) afforded the title compound. (80 mg, 71% yield). MS (m/z) 912.3 (M+H)⁺

### Intermediate 90: (S)-dibenzyl 2-(2-ethoxy-4-(5-(((4R,5R)-5-ethyl-6-formyl-10-methyl-3,8-dioxo-4-pentyl-10-phenyl-7-oxa-2,6,9-triazaundecyl)carbamoyl)furan-2-yl)benzamido)succinate

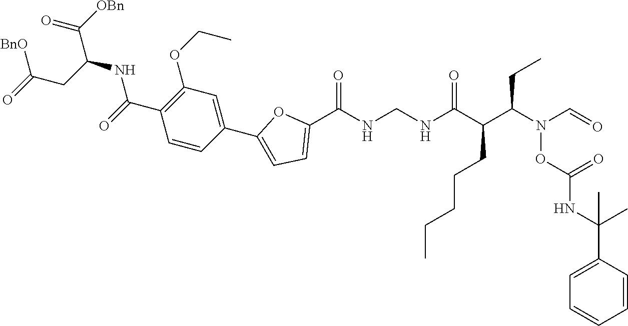 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)NC(C)(C)c1ccccc1]

(S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.1 g, 0.123 mmol) was added to a solution of and (2-isocyanatopropan-2-yl)benzene (0.099 g, 0.615 mmol) in DCM (1.230 mL) at 0° C. The reaction was stirred at RT overnight and concentrated. Purification by Si (20-100% EtOAc/Hex) afforded the title compound. (90 mg, 75% yield). MS (m/z) 974.3 (M+H)⁺

### Intermediate 91: (S)-dibenzyl 2-(2-ethoxy-4-(5-((((R)-2-((R)-1-(N-((3-(pyrrolidin-1-yl)benzoyl)oxy)formamido)propyl)heptanamido)methyl)carbamoyl)furan-2-yl)benzamido)succinate

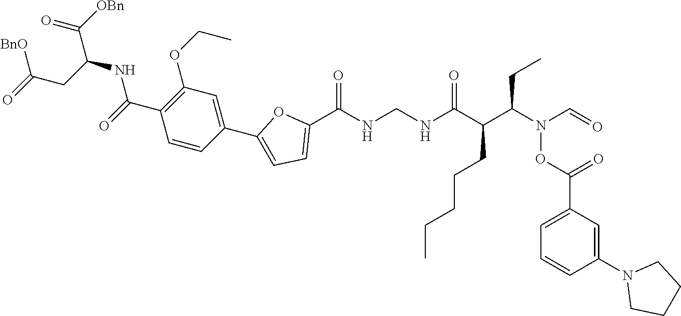 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1cccc(N2CCCC2)c1]

A mixture of 3-(pyrrolidin-1-yl)benzoic acid (0.039 g, 0.204 mmol), HATU (0.085 g, 0.224 mmol), and DIPEA (0.129 mL, 0.738 mmol) in MeCN (2 mL) was stirred at RT for 20 mins. (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.2 g, 0.246 mmol) was added and the reaction was stirred at RT overnight. Water was added and the reaction was extracted with EtOAc. The organic layer was dried over Na₂SO₄ and concentrated. Purification by Si (0-80% EtOAc/Hex) afforded the title compound as a light yellow foam. (72 mg, 36% yield). MS (m/z) 986.5 (M+H)⁺

### Intermediate 92: (S)-dibenzyl 2-(2-ethoxy-4-(5-((((R)-2-((R)-1-(N-((3-fluoro-4-(pyrrolidin-1-yl)benzoyl)oxy)formamido)propyl)heptanamido)methyl)carbamoyl)furan-2-yl)benzamido)succinate

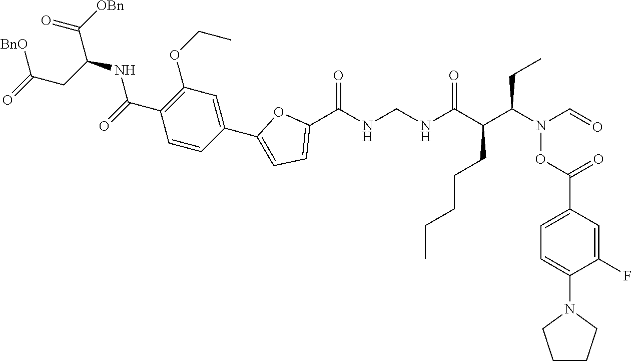 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(N2CCCC2)c(F)c1]

HATU (0.070 g, 0.184 mmol) was added to a solution of 3-fluoro-4-(pyrrolidin-1-yl)benzoic acid (0.035 g, 0.167 mmol) and DIPEA (0.129 mL, 0.738 mmol) in MeCN (2 mL). After stirring for 30 mins, (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.2 g, 0.246 mmol) was added and the reaction was stirred at RT overnight. Water was added and the reaction was extracted with EtOAc. The organic layer was dried over Na₂SO₄ and concentrated. Purification by Si (0-80% EtOAc/Hex) afforded the title compound as a light yellow solid. (74 mg, 43% yield).

### Intermediate 93: (S)-dibenzyl 2-(4-(5-((((R)-2-((R)-1-(N-((4-(1H-pyrrol-1-yl)benzoyl)oxy)formamido)propyl)heptanamido)methyl)carbamoyl)furan-2-yl)-2-ethoxybenzamido)succinate

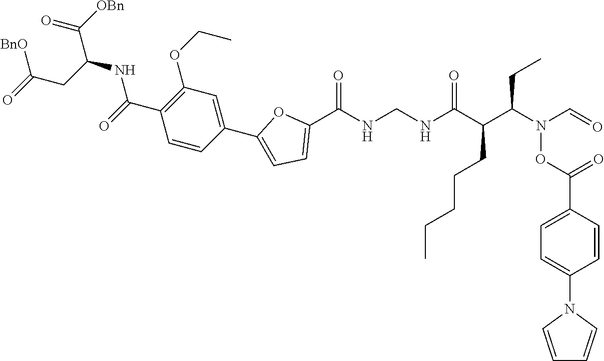 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(-n2cccc2)cc1]

HATU (0.077 g, 0.203 mmol) was added to a solution of 4-(1H-pyrrol-1-yl)benzoic acid (0.035 g, 0.185 mmol) and DIPEA (0.129 mL, 0.738 mmol) in MeCN (2 mL). After stirring for 20 mins, (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.2 g, 0.246 mmol) was added and the reaction was stirred at RT overnight. Water was added and the reaction was extracted with EtOAc. The organic layer was dried over Na₂SO₄ and concentrated. Purification by Si (0-80% EtOAc/Hex) afforded the title compound. (102 mg, 56% yield). MS (m/z) 982.5 (M+H)⁺

### Intermediate 94: (2S)-dibenzyl 2-(4-(5-((((2R)-2-((1R)-1-(N-((((benzyloxy)(hydroxy)phosphoryl)oxy)methoxy)formamido)propyl)heptanamido)methyl)carbamoyl)furan-2-yl)-2-ethoxybenzamido)succinate

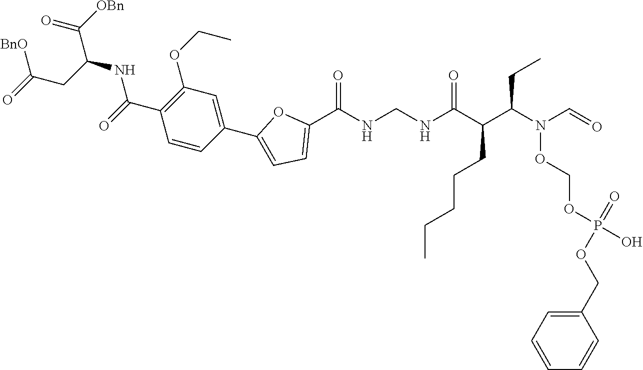 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OCOP(=O)(O)OCc1ccccc1]

### Step 1: (9H-fluoren-9-yl)methyl (((R)-2-((R)-1-(N-hydroxyformamido)propyl)heptanamido)methyl)carbamate

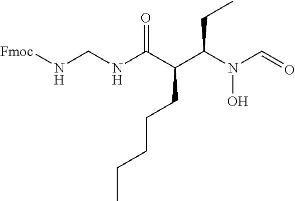

A solution containing (9H-fluoren-9-yl)methyl (((R)-2-((R)-1-(N-(benzyloxy)formamido)propyl)heptanamido)methyl)carbamate (9.9 g, 17.32 mmol) in MeOH (115 mL) was charged with Pd—C (1.843 g, 1.732 mmol) under Nitrogen. The solution was vacuum pumped and purged with Nitrogen 3×. Hydrogen was added via balloon. The reaction stirred for 1 h at RT. Additional MeOH:DCM was added to dissolve product which was precipitating out. Reaction was filtered and washed with MeOH. The filtrate was concentrated and taken up in MeOH/EtOAc and filtered again. Upon concentration, the residue was suspended in water and acidified with 1N HCl and then extracted into EtOAc 3×. The combined organic layers were washed with brine, dried over sodium sulfate, filtered and concentrated. Purification by Si-C18 reverse phase (20-60% MeCN/0.1% NH4OH in H2O) afforded the title compound. (3.8 g, 46% yield). MS (m/z) 482.2 (M+H)⁺

### Step 2: (9H-fluoren-9-yl)methyl (((2R)-2-((1R)-1-(N-((((benzyloxy)(hydroxy)phosphoryl)oxy)methoxy)formamido)propyl)heptanamido)methyl)carbamate

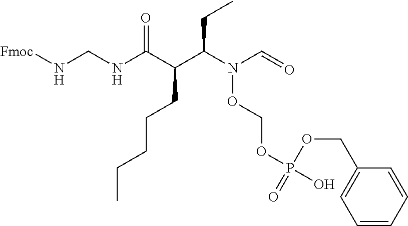

To a solution containing (9H-fluoren-9-yl)methyl (((R)-2-((R)-1-(N-hydroxyformamido)propyl)heptanamido)methyl)carbamate (5.0 g, 10.38 mmol) in THF (104 mL) was added dibenzyl (chloromethyl) phosphate (7.12 g, 21.80 mmol), sodium iodide (1.245 g, 8.31 mmol), and DIPEA (3.63 mL, 20.76 mmol). The reaction was stirred for 4 days in a sealed tube under nitrogen then filtered. The precipitate was washed with MeCN and the filtrate was then concentrated. Purification by Si-C18 reverse phase (20-70% MeCN/0.1% NH4OH in H2O) afforded the title compound as a yellow solid. (1 g, 14% yield). MS (m/z) 682.3 (M+H)⁺

### Step 3: ((N-((3R,4R)-4-((aminomethyl)carbamoyl)nonan-3-yl)formamido)oxy)methyl Benzyl Hydrogen Phosphate

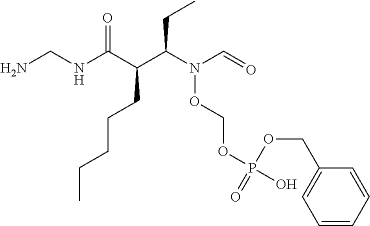 [SMILES: CCCCC[C@@H](C(=O)NCN)[C@@H](CC)N(C=O)OCOP(=O)(O)OCc1ccccc1]

To a solution containing (9H-fluoren-9-yl)methyl (((2R)-2-((1R)-1-(N-((((benzyloxy)(hydroxy)phosphoryl)oxy)methoxy)formamido)propyl)heptanamido)methyl)carbamate (1.8 g, 2.64 mmol) in DCM (26.4 mL) was added piperidine (0.660 mL, 6.60 mmol). The reaction was stirred for 18 h at RT under Nitrogen and concentrated. Purification by Si-C18 reverse phase (20-70% MeCN/0.1% NH4OH in H2O) afforded the title compound as a colorless solid. (600 mg, 50% yield). MS (m/z) 460.3 (M+H)⁺

### Step 4: (2S)-dibenzyl 2-(4-(5-((((2R)-2-((1R)-1-(N-((((benzyloxy)(hydroxy)phosphoryl)oxy)methoxy)formamido)propyl)heptanamido)methyl)carbamoyl)furan-2-yl)-2-ethoxybenzamido)succinate

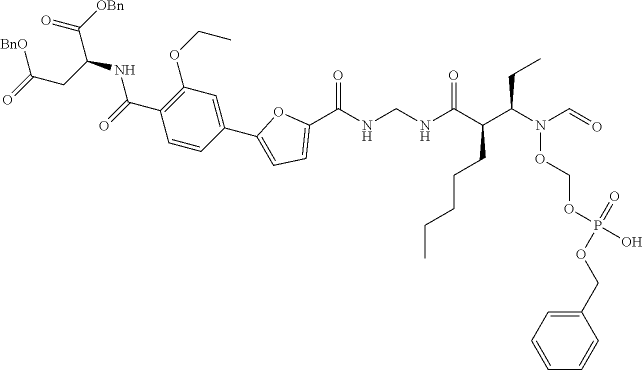 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OCOP(=O)(O)OCc1ccccc1]

To a solution containing ((N-((3R,4R)-4-((aminomethyl)carbamoyl)nonan-3-yl)formamido)oxy)methyl benzyl hydrogen phosphate (600 mg, 1.306 mmol) and (S)-5-(4-((1,4-bis(benzyloxy)-1,4-dioxobutan-2-yl)carbamoyl)-3-ethoxyphenyl)furan-2-carboxylic acid (896 mg, 1.567 mmol) in DMF (13 mL) was added EDC (751 mg, 3.92 mmol), 1-hydroxy-7-azabenzotriazole (533 mg, 3.92 mmol), and N-methylmorpholine (0.72 mL, 6.53 mmol). The reaction was stirred in for 18 h at RT under nitrogen. The reaction was poured into water and acidified to pH=4 with 1N HCl and extracted into EtOAc (4×100 mL). The combined organic layers were dried over sodium sulfate, filtered, and concentrated. Purification by Si-C18 reverse phase (10-70% MeCN/0.1% NH4OH in H2O) afforded the title compound. (572 mg, 43% yield). MS (m/z) 1013.2 (M+H)⁺

### Intermediate 95: Dibenzyl (3-(5-((((R)-2-((R)-1-(N-((2,3-dihydrobenzo[b][1,4]dioxine-6-carbonyl)oxy)formamido)propyl)heptanamido)methyl)carbamoyl)furan-2-yl)-5-ethoxyphenyl)phosphonate

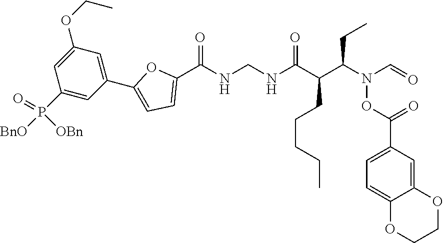 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc2c(c1)OCCO2]

HATU (0.093 g, 0.245 mmol) was added to a solution containing 2,3-dihydrobenzo[b][1,4]dioxine-6-carboxylic acid (0.041 g, 0.225 mmol) and DIPEA (0.107 mL, 0.613 mmol) in MeCN (1.5 mL). After stirring for 30 mins, dibenzyl (3-ethoxy-5-(5-((((R)-2-((R)-1-(N-hydroxyformamido)propyl)heptanamido)methyl)carbamoyl)furan-2-yl)phenyl)phosphonate (0.15 g, 0.204 mmol) was added and the reaction was stirred at RT overnight. Water was added and the reaction was partitioned between water and EtOAc. The organic layer was dried over Na₂SO₄ and concentrated. Purification by Si (0-80% EtOAc/Hex) afforded the title compound as a pale yellow foam. (177 mg, 97% yield). MS (m/z) 896.4 (M+H)⁺

### Intermediate 96: Dibenzyl (3-ethoxy-5-(5-(((4R,5R)-5-ethyl-6-formyl-10,10-dimethyl-3,8-dioxo-4-pentyl-7,9-dioxa-2,6-diazaundecyl)carbamoyl)furan-2-yl)phenyl)phosphonate

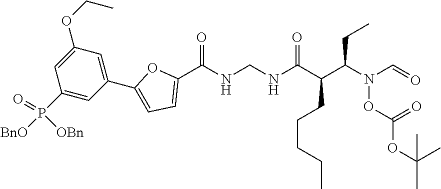 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)OC(C)(C)C]

TEA (42.7 μl, 0.307 mmol) was added to a solution containing dibenzyl (3-ethoxy-5-(5-((((R)-2-((R)-1-(N-hydroxyformamido)propyl)heptanamido)methyl)carbamoyl)furan-2-yl)phenyl)phosphonate (75 mg, 0.102 mmol) and Boc₂O (35.6 μl, 0.153 mmol) in MeCN (1022 μl). The reaction was stirred for 30 min and then concentrated. Purification by Si (0-100% EtOAc/Hex) afforded the title compound. (72 mg, 84% yield). MS (m/z) 934.4 (M+H)⁺

### Intermediate 97: (S)-dibenzyl 2-(4-(5-((((R)-2-((R)-1-(N-((2,3-dihydrobenzo[b][1,4]dioxine-6-carbonyl)oxy)formamido)propyl)heptanamido)methyl)carbamoyl)furan-2-yl)-2-ethoxybenzamido)succinate

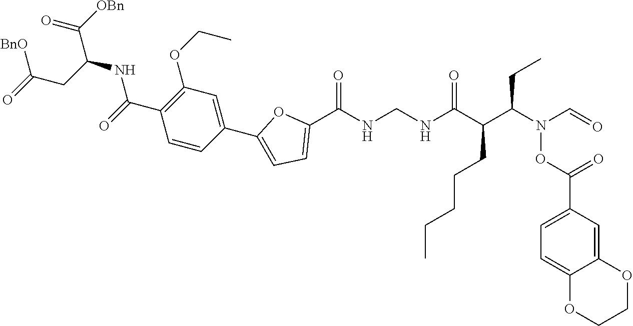 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc2c(c1)OCCO2]

HATU (0.084 g, 0.221 mmol) was added to a solution containing 2,3-dihydrobenzo[b][1,4]dioxine-6-carboxylic acid (0.037 g, 0.203 mmol) and DIPEA (0.097 mL, 0.554 mmol) in MeCN (2 mL) at RT. After stirring for 30 mins, (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.2 g, 0.185 mmol), was added and the reaction was stirred at RT overnight. Water was added and the reaction was extracted with EtOAc. The organic layer was dried over Na₂SO₄ and concentrated. Purification by Si (0-80% EtOAc/Hex) afforded the title compound as a pale yellow solid. (162 mg, 90% yield). MS (m/z) 975.4 (M+H)⁺

### Intermediate 98: Dibenzyl (3-ethoxy-5-(5-((((R)-2-((R)-1-(N-((4-methyl-3,4-dihydro-2H-benzo[b][1,4]oxazine-6-carbonyl)oxy)formamido)propyl)heptanamido)methyl)carbamoyl)furan-2-yl)phenyl)phosphonate

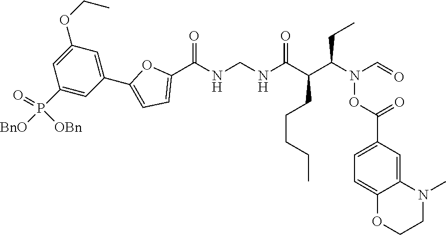 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc2c(c1)N(C)CCO2]

HATU (0.093 g, 0.245 mmol) was added to a solution of 4-methyl-3,4-dihydro-2H-benzo[b][1,4]oxazine-6-carboxylic acid (0.043 g, 0.225 mmol) and DIPEA (0.107 mL, 0.613 mmol) in MeCN (1.5 mL). After stirring for 30 mins, dibenzyl (3-ethoxy-5-(5-((((R)-2-((R)-1-(N-hydroxyformamido)propyl)heptanamido)methyl)carbamoyl)furan-2-yl)phenyl)phosphonate (0.15 g, 0.204 mmol) was added and the reaction was stirred at RT overnight. Water was added and the reaction was partitioned between water and EtOAc. The organic layer was dried over Na₂SO₄ and concentrated. Purification by Si (0-80% EtOAc/Hex) afforded the title compound as a pale yellow solid. (159 mg, 86% yield). MS (m/z) 909.4 (M+H)⁺

### Intermediate 99: (S)-dibenzyl 2-(2-ethoxy-4-(5-((((R)-2-((R)-1-(N-((4-methyl-3,4-dihydro-2H-benzo[b][1,4]oxazine-6-carbonyl)oxy)formamido)propyl)heptanamido)methyl)carbamoyl)furan-2-yl)benzamido)succinate

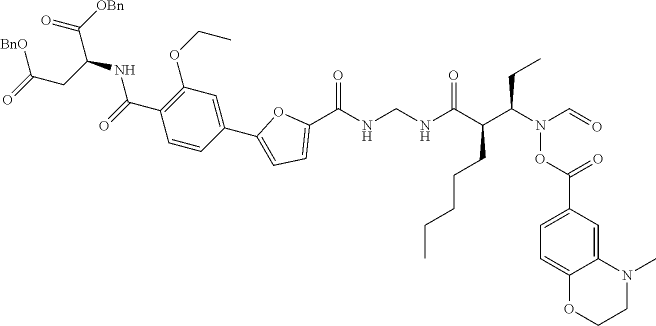 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc2c(c1)N(C)CCO2]

HATU (0.093 g, 0.245 mmol) was added to a solution of 4-methyl-3,4-dihydro-2H-benzo[b][1,4]oxazine-6-carboxylic acid (0.043 g, 0.225 mmol) and DIPEA (0.107 mL, 0.613 mmol) in MeCN (1.5 mL). After stirring for 30 mins, (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.208 g, 0.204 mmol) was added and the reaction was stirred at RT overnight. Water was added and the reaction was partitioned between water and EtOAc. The organic layer was dried over Na₂SO₄ and concentrated. Purification by Si (0-80% EtOAc/Hex) afforded the title compound as a yellow foam. (76 mg, 38% yield). MS (m/z) 989.6 (M+H)⁺

### Intermediate 100: (S)-dibenzyl 2-(2-ethoxy-4-(5-(((4R,5R)-5-ethyl-6-formyl-10,10-dimethyl-3,8-dioxo-4-pentyl-7,9-dioxa-2,6-diazaundecyl)carbamoyl)furan-2-yl)benzamido)succinate

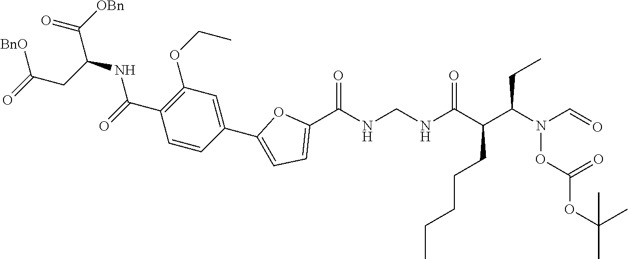 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)OC(C)(C)C]

TEA (38.6 μl, 0.277 mmol) was added to a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (75 mg, 0.092 mmol) and Boc₂O (32.1 μl, 0.138 mmol) in MeCN (923 μl). The resulting mixture was stirred for 30 min and was then concentrated. Purification by Si (0-100% EtOAc/Hex) afforded the title compound. (29 mg, 34% yield). MS (m/z) 913.5 (M+H)⁺

### Intermediate 101: (S)-dibenzyl 2-(2-(2-(benzyloxy)-2-oxoethoxy)-4-(5-((((R)-2-((R)-1-(N-((2-methoxy-6-methylbenzoyl)oxy)formamido)propyl)heptanamido)methyl)carbamoyl)furan-2-yl)benzamido)succinate

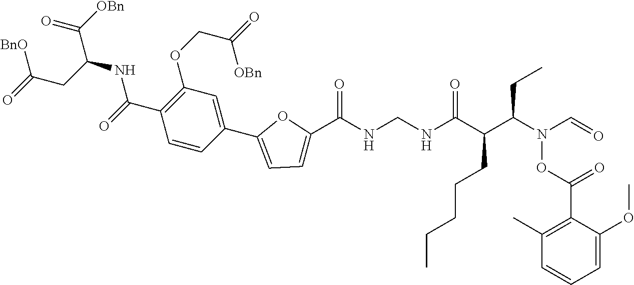 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)c1c(C)cccc1OC]

DIPEA (84 μl, 0.482 mmol) was added to a solution containing (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (150 mg, 0.161 mmol) and 2-methoxy-6-methylbenzoic acid (29.4 mg, 0.177 mmol), and HATU (73.4 mg, 0.193 mmol) in DMF (1072 μl). The resulting mixture was stirred for 2 days at RT. Purification by reverse phase HPLC (MeCN:Water TFA 50-100%) afforded the title compound as a yellow solid. (62 mg, 36% yield). MS (m/z) 1081.6 (M+H)⁺

### Intermediate 102: Dibenzyl (3-ethoxy-5-(5-((((R)-2-((R)-1-(N-((4-methoxy-2-methylbenzoyl)oxy)formamido)propyl)heptanamido)methyl)carbamoyl)furan-2-yl)phenyl)phosphonate

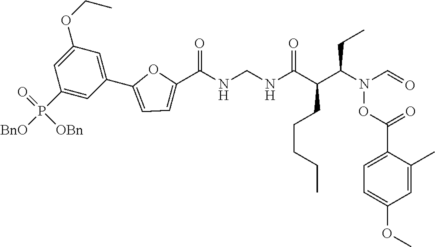 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(OC)cc1C]

HATU (0.114 g, 0.300 mmol) was added to a solution containing 4-methoxy-2-methylbenzoic acid (0.045 g, 0.273 mmol) and DIPEA (0.143 mL, 0.818 mmol) in MeCN (1.5 mL). After stirring for 30 mins, dibenzyl (3-ethoxy-5-(5-((((R)-2-((R)-1-(N-hydroxyformamido)propyl)heptanamido)methyl)carbamoyl)furan-2-yl)phenyl)phosphonate (0.2 g, 0.273 mmol) was added and the reaction was stirred at RT overnight. Water was added and the reaction was partitioned between water and EtOAc. The organic layer was dried over Na₂SO₄ and concentrated. Purification by Si (0-85% EtOAc/Hex) afforded the title compound as a colorless foam. (165 mg, 69% yield). MS (m/z) 882.4 (M+H)⁺

### Intermediate 103: Dibenzyl (3-ethoxy-5-(5-((((R)-2-((R)-1-(N-((2-isopropyl-4-methoxybenzoyl)oxy)formamido)propyl)heptanamido)methyl)carbamoyl)furan-2-yl)phenyl)phosphonate

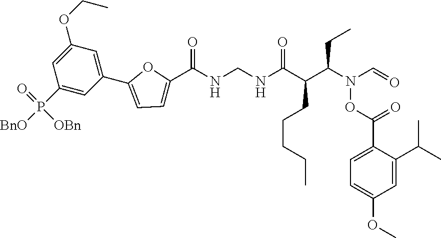 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(OC)cc1C(C)C]

Oxalyl dichloride (2 M in DCM) (335 μl, 0.669 mmol) was slowly added to a solution containing 2-isopropyl-4-methoxybenzoic acid (100 mg, 0.515 mmol) and DMF (3.99 μl, 0.051 mmol) in DCM (1716 μl) at 0° C. The resulting mixture was stirred for 18 h as it warmed to RT. The mixture was then concentrated to dryness to give 2-isopropyl-4-methoxybenzoyl chloride. The crude product was cooled to 0° C., and a solution of dibenzyl (3-ethoxy-5-(5-((((R)-2-((R)-1-(N-hydroxyformamido)propyl)heptanamido)methyl)carbamoyl)furan-2-yl)phenyl)phosphonate (94 mg, 0.129 mmol) in MeCN (2 mL) was added followed by TEA (215 μl, 1.545 mmol). The resulting solution was allowed to come to RT over 18 h and concentrated. Purification by Si (0-100% EtOAc/Hex) afforded the title compound. (96 mg, 20% yield). MS (m/z) 910.5 (M+H)⁺

### Intermediate 104: (S)-dibenzyl 2-(2-(2-(benzyloxy)-2-oxoethoxy)-4-(5-((((R)-2-((R)-1-(N-((phenylcarbamoyl)oxy)formamido)propyl)heptanamido)methyl)carbamoyl)furan-2-yl)benzamido)succinate

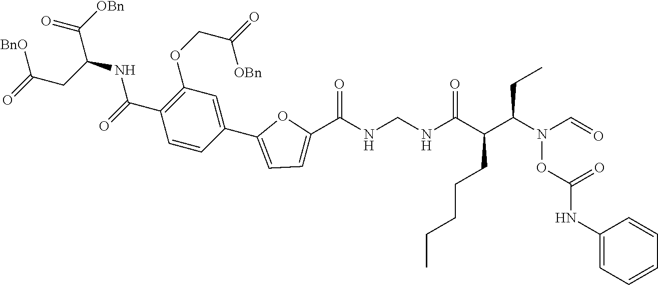 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)Nc1ccccc1]

Bis(trichloromethyl) carbonate (23.54 mg, 0.079 mmol) was dissolved in DCM (1 mL) and cooled to 0° C. TEA (0.066 mL, 0.472 mmol) and aniline (0.020 mL, 0.214 mmol) in DCM (1 mL) was added drop wise. The mixture was stirred at RT for 15 min and (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (200 mg, 0.214 mmol) in DCM (1 mL) was added at 0° C. The solution was stirred at 0° C. for 10 min, followed by addition of TEA (0.066 mL, 0.472 mmol). The reaction mixture was allowed to warm to RT over 18 h and concentrated. Purification by Si (0-100% EtOAc/Hex) afforded the title compound as a yellow oil. (100 mg, 44% yield). MS (m/z) 1053.4 (M+2H)+

### Intermediate 105: (S)-dibenzyl 2-(4-(5-((((R)-2-((R)-1-(N-((3,4-dimethoxybenzoyl)oxy)formamido)propyl)heptanamido)methyl)carbamoyl)furan-2-yl)-2-ethoxybenzamido)succinate

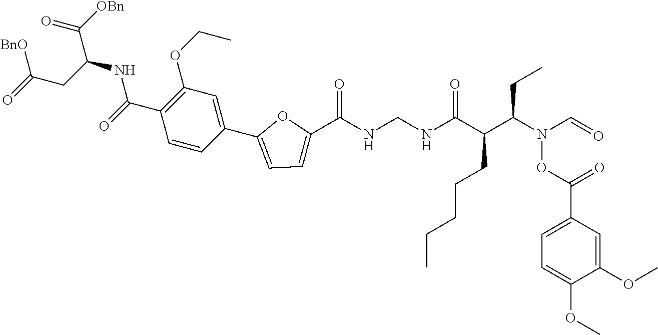 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(OC)c(OC)c1]

Methanesulfonyl chloride (0.013 mL, 0.162 mmol) was added to a solution of 1-methylimidazole (0.024 mL, 0.295 mmol) and 3,4-dimethoxybenzoic acid (0.027 g, 0.148 mmol) in DCM (2 mL) at 0° C. After stirring for 30 mins at 0° C., (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.12 g, 0.148 mmol) was added and the reaction was stirred at RT overnight and concentrated. Purification by Si (0-100% EtOAc/Hex) afforded the title compound. (70 mg, 49% yield). MS (m/z) 977.5 (M+H)⁺

### Intermediate 106: Dibenzyl (3-ethoxy-5-(5-((((R)-2-((R)-1-(N-((phenylcarbamoyl)oxy)formamido)propyl)heptanamido)methyl)carbamoyl)furan-2-yl)phenyl)phosphonate

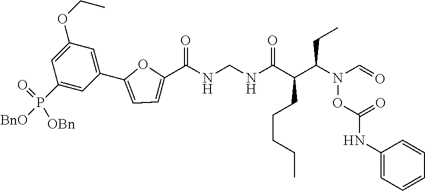 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)Nc1ccccc1]

Triphosgene (0.030 g, 0.101 mmol) was dissolved in DCM (1.363 mL) and cooled to 0° C. Aniline (0.025 mL, 0.273 mmol) and TEA (0.084 mL, 0.600 mmol) in DCM (1.4 mL) were added drop wise. The mixture was stirred at RT for 15 min and dibenzyl (3-ethoxy-5-(5-((((R)-2-((R)-1-(N-hydroxyformamido)propyl)heptanamido)methyl)carbamoyl)furan-2-yl)phenyl)phosphonate (0.1 g, 0.136 mmol) in DCM (1.4 mL) was added at 0° C. The solution was stirred at 0° C. for 10 min. The reaction mixture was allowed to warm to RT for 18 h. The reaction mixture was concentrated. Purification by Si (0-100% EtOAc/Hex) afforded the title compound as a yellow oil. (80 mg, 69% yield). MS (m/z) 853.2 (M+H)⁺

### Intermediate 107: (S)-dibenzyl 2-(2-ethoxy-4-(5-((((R)-2-((R)-1-(N-((2-methylbenzoyl)oxy)formamido)propyl)heptanamido)methyl)carbamoyl)furan-2-yl)benzamido)succinate

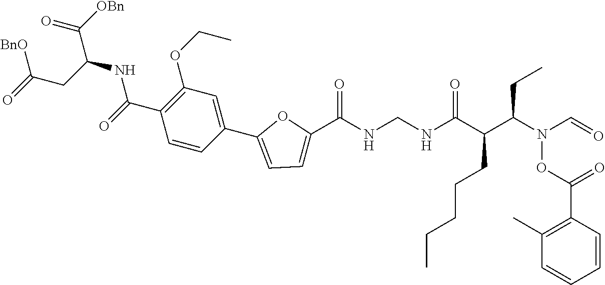 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccccc1C]

TEA (0.086 mL, 0.615 mmol) was added to a stirring solution of (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.1 g, 0.123 mmol) and 2-methylbenzoyl chloride (0.080 mL, 0.615 mmol) in MeCN (2 mL). The resulting mixture was stirred for 15 min and concentrated. Purification by Si (0-100% EtOAc/Hex) afforded the title compound as a colorless foam. (22 mg, 21% yield). MS (m/z) 931.6 (M+H)⁺

### Intermediate 108: (S)-dibenzyl 2-(2-ethoxy-4-(5-(((4R,5R)-5-ethyl-6-formyl-11,11-dimethyl-3,10-dioxo-4-pentyl-7,9-dioxa-2,6-diazadodecyl)carbamoyl)furan-2-yl)benzamido)succinate

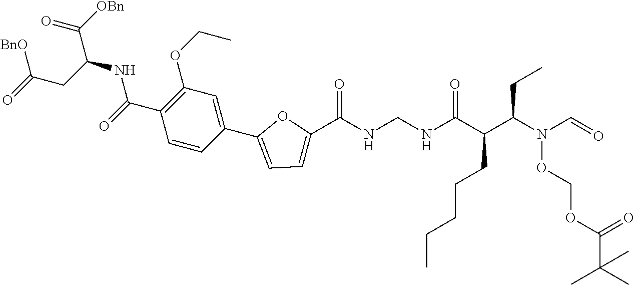 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OCOC(=O)C(C)(C)C]

(S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.15 g, 0.185 mmol) was suspended in THF (2 mL). DIPEA (0.097 mL, 0.554 mmol) and iodomethyl pivalate (0.089 g, 0.369 mmol) were added and stirred for 12 h. DIPEA (0.097 mL, 0.554 mmol) and iodomethyl pivalate (0.089 g, 0.369 mmol) were added again and stirred for 3 h and concentrated. Purification by Si (0-100% EtOAc/Hex) afforded the title compound as a yellow oil. (80 mg, 23% yield). MS (m/z) 927.6 (M+H)⁺

### Intermediate 109: Dibenzyl N-({2-[2-(benzyloxy)-2-oxoethoxy]-4-(5-{[(4R,5R)-5-ethyl-6-formyl-9,12,12-trimethyl-3,8,10-trioxo-4-pentyl-7,11-dioxa-2,6,9-triazatridec-1-yl]carbamoyl}furan-2-yl)phenyl}carbonyl)-L-aspartate

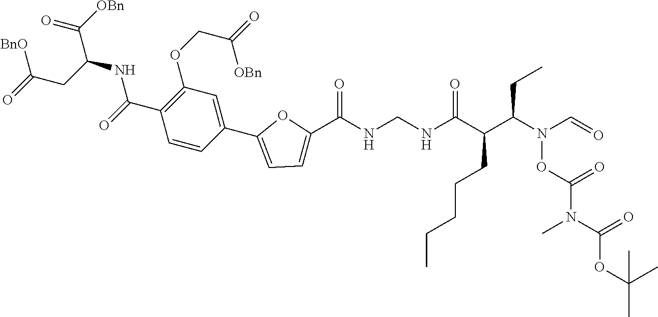 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC(=O)OCc3ccccc3)c2)o1)[C@@H](CC)N(C=O)OC(=O)N(C)C(=O)OC(C)(C)C]

To a solution of (S)-dibenzyl 2-(2-(2-(benzyloxy)-2-oxoethoxy)-4-(5-((((R)-2-((R)-1-(N-hydroxyformamido)propyl)heptanamido)methyl)carbamoyl)furan-2-yl)benzamido)succinate (0.1 g, 0.107 mmol), and DIPEA (0.056 mL, 0.322 mmol) in THF (0.5 mL) was slowly added N-methyl-Boc chloromethyl (0.072 g, 0.322 mmol). The reaction was stirred for 18 h and concentrated. Purification by Si (0-100% EtOAc/Hex) afforded the title compound as a colorless foam. (50 mg, 43% yield). MS (m/z) 1090.5 (M+H)⁺

### Intermediate 110: Chloromethyl N-methyl Carbamate

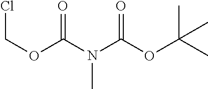

A solution of DMAP (0.052 g, 0.427 mmol), pyridine (2.59 mL, 32.0 mmol), and tert-butyl methylcarbamate (2.8 g, 21.35 mmol) in DCM (25 mL) was slowly added to a stirring solution 0° C. of chloromethyl carbonochloridate (1.993 mL, 22.41 mmol) in DCM (107 mL). The solution was allowed to warm to RT over 1.5 h and concentrated. Purification by Si (0-100% EtOAc/Hex) afforded the title compound as a clear oil. (2.56 g, 54% yield).

### Intermediate 111: (S)-dibenzyl 2-(4-(5-((((R)-2-((R)-1-(N-((2,4-dimethoxybenzoyl)oxy)formamido)propyl)heptanamido)methyl)carbamoyl)furan-2-yl)-2-ethoxybenzamido)succinate

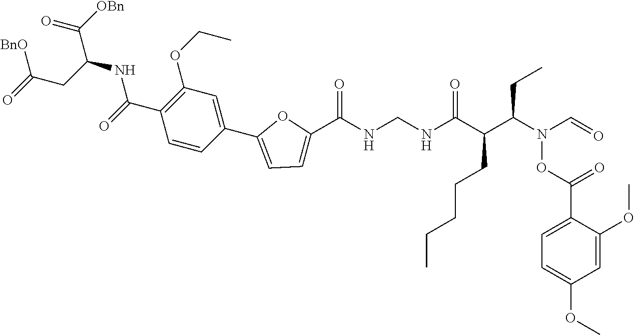 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(OC)cc1OC]

TEA (0.070 mL, 0.499 mmol) was added to a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.12 g, 0.148 mmol) and 2,4-dimethoxybenzoyl chloride (0.1 g, 0.498 mmol) in MeCN (4 mL). The resulting mixture was stirred for 15 min and concentrated. Purification by Si (0-100% EtOAc/Hex) afforded the title compound as a colorless foam. (60 mg, 42% yield). MS (m/z) 977.5 (M+H)⁺

### Intermediate 112: Dibenzyl (3-ethoxy-5-(5-((((R)-2-((R)-1-(N-((2-methyl-4-morpholinobenzoyl)oxy)formamido)propyl)heptanamido)methyl)carbamoyl)furan-2-yl)phenyl)phosphonate

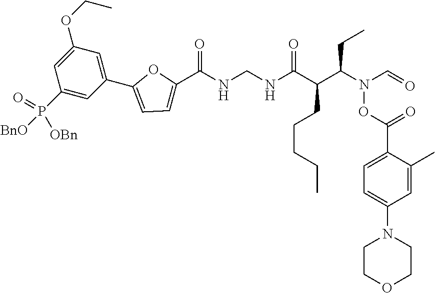 [SMILES: CCCCC[C@@H](C(=O)NCNC(=O)c1ccc(-c2cc(OCC)cc(P(=O)(OCc3ccccc3)OCc3ccccc3)c2)o1)[C@@H](CC)N(C=O)OC(=O)c1ccc(N2CCOCC2)cc1C]

A suspension of HATU (0.093 g, 0.245 mmol), 2-methyl-4-morpholinobenzoic acid (0.045 g, 0.204 mmol) and DIPEA (0.107 mL, 0.613 mmol) in MeCN (1.5 mL) was stirred for 30 mins. dibenzyl (3-ethoxy-5-(5-((((R)-2-((R)-1-(N-hydroxyformamido)propyl)heptanamido)methyl)carbamoyl)furan-2-yl)phenyl)phosphonate (0.15 g, 0.204 mmol) was added and the reaction was stirred at RT for 18 h. Water was added and the reaction was partitioned between water and EtOAc. The organic layer was dried over Na₂SO₄ and concentrated. Purification by Si (0-80% EtOAc/Hex) afforded the title compound as a colorless foam. (121 mg, 63% yield). MS (m/z) 937.4 (M+H)⁺

### Intermediate 113: 2-methyl-4-morpholinobenzoic Acid

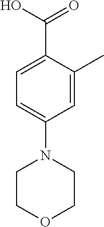 [SMILES: Cc1cc(N2CCOCC2)ccc1C(=O)O]

### Step 1: Methyl 2-methyl-4-morpholinobenzoate

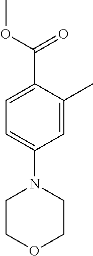 [SMILES: COC(=O)c1ccc(N2CCOCC2)cc1C]

Tris(dibenzylideneacetone)dipalladium(0) (0.2 g, 0.218 mmol) was added to a degassed suspension of methyl 4-bromo-2-methylbenzoate (1 g, 4.37 mmol), cesium carbonate (2 g, 6.14 mmol), morpholine (0.570 mL, 6.55 mmol), and (R)-(+)-2,2′-bis(diphenylphosphino)-1,1′-binaphthyl (0.2 g, 0.321 mmol) in toluene (10 mL). The reaction was stirred under N₂ for 15 min before it was stirred at 110° C. for 6 h. The reaction was cooled and water was added and the reaction was extracted with EtOAc. The organic layer was dried over Na₂SO₄ and concentrated.

Purification by Si (0-20% EtOAc/Hex) afforded the title compound as a colorless solid. (857 mg, 83% yield). MS (m/z) 236.2 (M+H)⁺

### Step 2: 2-methyl-4-morpholinobenzoic Acid

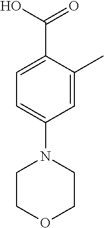 [SMILES: Cc1cc(N2CCOCC2)ccc1C(=O)O]

A mixture of methyl 2-methyl-4-morpholinobenzoate (0.857 g, 3.64 mmol) and lithium hydroxide (0.420 g, 17.54 mmol) in THF (14 mL) and Water (7 mL) was stirred at 50° C. overnight. The reaction was cooled down and concentrated. The residue was partitioned between EtOAc and water. The water layer was acidified with 6N HCl until pH˜5. The resulted solid was collected by filtration, washed with water and air dried to afford the title compound as a pale grey solid. (720 mg, 89% yield). MS (m/z) 222.1 (M+H)⁺

### Intermediate 114: (S)-dibenzyl 2-(2-ethoxy-4-(5-((((R)-2-((R)-1-(N-((2-isopropylbenzoyl)oxy)formamido)propyl)heptanamido)methyl)carbamoyl)furan-2-yl)benzamido)succinate

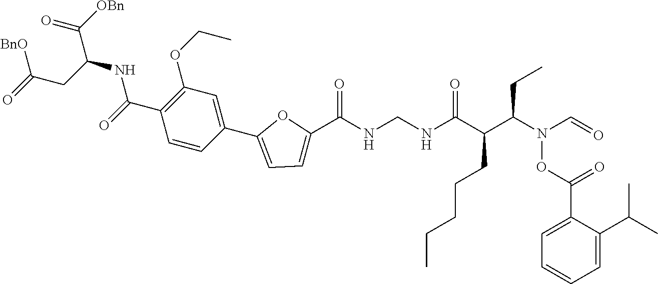 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccccc1C(C)C]

DIPEA (0.081 mL, 0.461 mmol) was added to a solution containing 2-isopropylbenzoic acid (0.025 g, 0.154 mmol), HATU (0.070 g, 0.185 mmol), and (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.125 g, 0.154 mmol) in DMF (1 mL). The mixture was stirred at 25° C. for 18 h and was then concentrated. Purification by Si (0-100% EtOAc/Hex) afforded the title compound. (60 mg, 41% yield). MS (m/z) 959.5 (M+H)⁺

### Intermediate 115: (S)-dibenzyl 2-(4-(5-((((R)-2-((R)-1-(N-((2,4-dimethylnicotinoyl)oxy)formamido)propyl)heptanamido)methyl)carbamoyl)furan-2-yl)-2-ethoxybenzamido)succinate

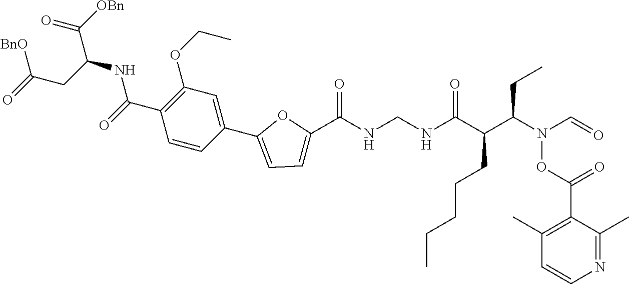 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1c(C)ccnc1C]

DIPEA (0.081 mL, 0.461 mmol) was added to a solution containing 2,4-dimethylnicotinic acid (0.026 g, 0.169 mmol), HATU (0.070 g, 0.185 mmol), and (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.125 g, 0.154 mmol) in DMF (1 mL). The mixture was stirred at 25° C. for 18 h and was then concentrated. Purification by Si (0-100% EtOAc/Hex) afforded the title compound. (80 mg, 55% yield). MS (m/z) 946.5 (M+H)⁺

### Intermediate 116: Ethyl 4-(5-((((R)-2-((R)-1-(N-(benzyloxy)formamido)propyl)heptanamido)methyl)carbamoyl)furan-2-yl)benzoate

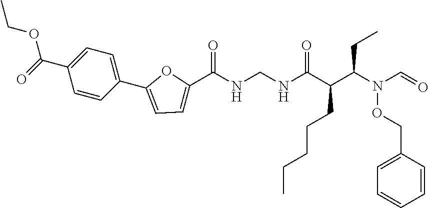 [SMILES: CCCCC[C@@H](C(=O)NCNC(=O)c1ccc(-c2ccc(C(=O)OCC)cc2)o1)[C@@H](CC)N(C=O)OCc1ccccc1]

### Step 1: Ethyl 4-(4,4,5,5-tetramethyl-1,3,2-dioxaborolan-2-yl)benzoate

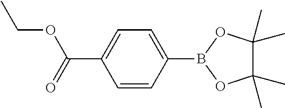 [SMILES: CCOC(=O)c1ccc(B2OC(C)(C)C(C)(C)O2)cc1]

Ethyl 4-bromobenzoate (0.356 mL, 2.183 mmol), bis(pinacolato)diboron (0.665 g, 2.62 mmol), potassium acetate (0.857 g, 8.73 mmol), PdCl2(dppf)-CH2Cl2 (0.089 g, 0.109 mmol) in 1,4-dioxane (20 mL) was placed in a microwave vial. The vial was sealed and heated to 140° C. for 20 min in μwave. The reaction was diluted with EtOAc (100 mL) and brine (100 mL) and passed through a pad of celite. The organic phase was passed through a phase separator and the organic phase was concentrated. Purification by Si (0-100% EtOAc/Hex) afforded the title compound as a yellow oil. (876 mg, 67% yield). MS (m/z) 277.2 (M+H)⁺

### Step 2: Ethyl 4-(5-((((R)-2-((R)-1-(N-(benzyloxy)formamido)propyl)heptanamido)methyl)carbamoyl)furan-2-yl)benzoate

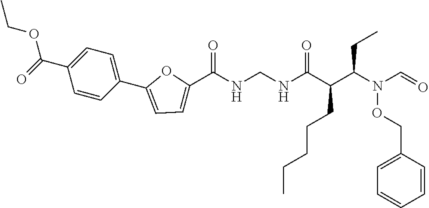 [SMILES: CCCCC[C@@H](C(=O)NCNC(=O)c1ccc(-c2ccc(C(=O)OCC)cc2)o1)[C@@H](CC)N(C=O)OCc1ccccc1]

To a microwave vial was added a solution containing ethyl 4-(4,4,5,5-tetramethyl-1,3,2-dioxaborolan-2-yl)benzoate (0.876 g, 3.17 mmol) in 1,4-Dioxane (15 mL) along with N—(((R)-2-((R)-1-(N-(benzyloxy)formamido)propyl)heptanamido)methyl)-5-bromofuran-2-carboxamide (1.35 g, 2.58 mmol), Na2CO3 (1M in water) (7.75 mL, 7.75 mmol), and PdCl2(dppf)-CH2Cl2 (0.106 g, 0.129 mmol). The reaction was heated to 100° C. for 6 min in μwave.

The reaction mixture was diluted with brine (40 mL) and EtOAc (80 mL) and water (80 mL) and filtered through a pad of celite. The phases were separated, passed through a phase separator, filtered, and concentrated. Purification by Si (0-100% EtOAc/Hex) afforded the title compound as a pale pink solid. (892 mg, 55% yield). MS (m/z) 592.3 (M+H)⁺

### Intermediate 117: N—(((R)-2-((R)-1-(N-(benzyloxy)formamido)propyl)heptanamido)methyl)-5-bromofuran-2-carboxamide

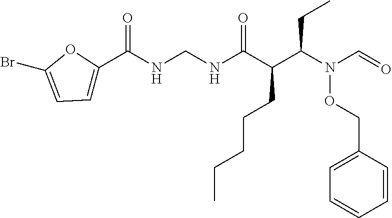 [SMILES: CCCCC[C@@H](C(=O)NCNC(=O)c1ccc(Br)o1)[C@@H](CC)N(C=O)OCc1ccccc1]

To a solution containing (R)—N-(aminomethyl)-2-((R)-1-(N-(benzyloxy)formamido)propyl)heptanamide (10.67 g, 30.5 mmol) in DMF (69.4 mL) was prepared. A solution containing 5-bromofuran-2-carboxylic acid (5.30 g, 27.8 mmol), HATU (12.66 g, 33.3 mmol), and DIPEA (14.54 mL, 83 mmol) in MeCN (69.4 mL) was stirred for 30 min at RT and was then slowly added to the amine solution. The mixture was stirred for 1 h at RT and EtOAc was added. The organic phase was washed with saturated NaHCO₃ (2×) and brine (1×). The combined washes were back extracted using EtOAc (1×). The combined organic phase was dried over MgSO4, filtered, and concentrated. Purification by Si (0-100% EtOAc/Hex) afforded the title compound. (14.1 g, 87% yield). MS (m/z) 522.2 (M+H)⁺

### Intermediate 118: Butyl 4-(5-((((R)-2-((R)-1-(N-(benzyloxy)formamido)propyl)heptanamido)methyl)carbamoyl)furan-2-yl)benzoate

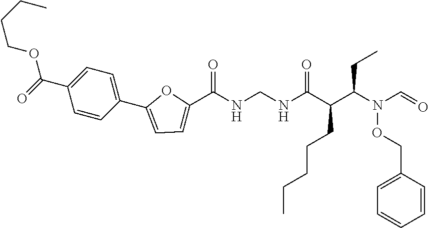 [SMILES: CCCCC[C@@H](C(=O)NCNC(=O)c1ccc(-c2ccc(C(=O)OCCCC)cc2)o1)[C@@H](CC)N(C=O)OCc1ccccc1]

### Step 1: 4-(5-((((R)-2-((R)-1-(N-(benzyloxy)formamido)propyl)heptanamido)methyl)carbamoyl)furan-2-yl)benzoic Acid

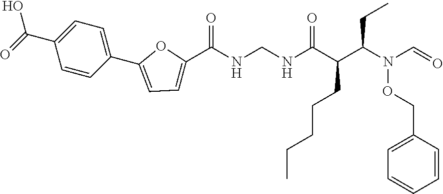 [SMILES: CCCCC[C@@H](C(=O)NCNC(=O)c1ccc(-c2ccc(C(=O)O)cc2)o1)[C@@H](CC)N(C=O)OCc1ccccc1]

To a solution containing ethyl 4-(5-((((R)-2-((R)-1-(N-(benzyloxy)formamido)propyl)heptanamido)methyl)carbamoyl)furan-2-yl)benzoate (2 g, 3.38 mmol) in EtOH (16.00 mL) and water (4 mL) was added LiOH (0.243 g, 10.14 mmol). Reaction was vortexed to dissolve and stirred at RT for 3.5 h at which time reaction was concentrated. The aqueous layer was acidified to ˜pH 4 and extracted into EtOAc (3×). The combined organic layers were passed through a phase separator and concentrated. Purification by Si (0-10% MeOH/EtOAc) afforded the title compound as an off white solid. (1.7 g, 89% yield). MS (m/z) 564.3 (M+H)⁺

### Step 2: Butyl 4-(5-((((R)-2-((R)-1-(N-(benzyloxy)formamido)propyl)heptanamido)methyl)carbamoyl)furan-2-yl)benzoate

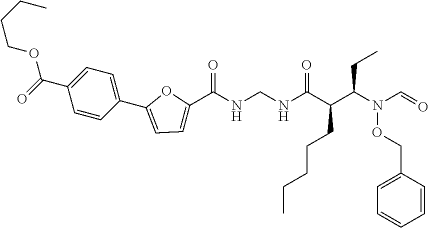 [SMILES: CCCCC[C@@H](C(=O)NCNC(=O)c1ccc(-c2ccc(C(=O)OCCCC)cc2)o1)[C@@H](CC)N(C=O)OCc1ccccc1]

To a solution containing 4-(5-((((R)-2-((R)-1-(N-(benzyloxy)formamido)propyl)heptanamido)methyl)carbamoyl)furan-2-yl)benzoic acid (100 mg, 0.177 mmol) in MeCN (1 mL) and DMF (0.2 mL) was added potassium carbonate (123 mg, 0.887 mmol) and 1-bromobutane (0.038 mL, 0.355 mmol). The reaction was stirred at 75° C. for 18 h at which time it was cooled, filtered, and concentrated. Purification by Si (0-100% EtOAc/Hex) afforded the title compound as an white solid. (24 mg, 22% yield). MS (m/z) 620.2 (M+H)⁺

### Intermediate 119: Phenethyl 4-(5-((((R)-2-((R)-1-(N-(benzyloxy)formamido)propyl)heptanamido)methyl)carbamoyl)furan-2-yl)benzoate

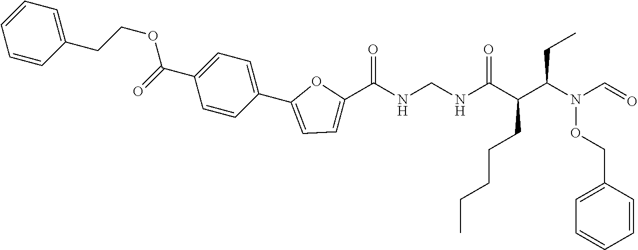 [SMILES: CCCCC[C@@H](C(=O)NCNC(=O)c1ccc(-c2ccc(C(=O)OCCc3ccccc3)cc2)o1)[C@@H](CC)N(C=O)OCc1ccccc1]

To a solution containing 4-(5-((((R)-2-((R)-1-(N-(benzyloxy)formamido)propyl)heptanamido)methyl)carbamoyl)furan-2-yl)benzoic acid (100 mg, 0.177 mmol) and potassium carbonate (123 mg, 0.887 mmol) in MeCN (1 mL) and DMF (0.2 mL) was added (2-bromoethyl)benzene (0.048 mL, 0.355 mmol). The reaction was stirred at 75° C. for 21 h and cooled to RT. The reaction was filtered and concentrated. Purification by Si (0-100% EtOAc/Hex) afforded the title compound as an white solid. (112 mg, 95% yield). MS (m/z) 668.2 (M+H)⁺

### Intermediate 120: 2-morpholinoethyl 4-(5-((((R)-2-((R)-1-(N-(benzyloxy)formamido)propyl)heptanamido)methyl)carbamoyl)furan-2-yl)benzoate

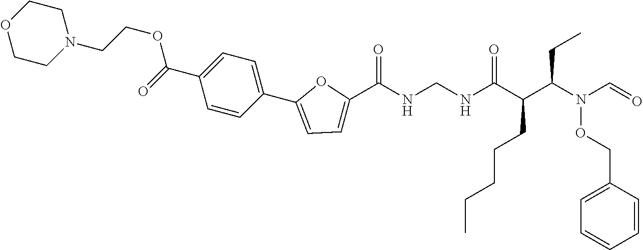 [SMILES: CCCCC[C@@H](C(=O)NCNC(=O)c1ccc(-c2ccc(C(=O)OCCN3CCOCC3)cc2)o1)[C@@H](CC)N(C=O)OCc1ccccc1]

To a solution containing 4-(5-((((R)-2-((R)-1-(N-(benzyloxy)formamido)propyl)heptanamido)methyl)carbamoyl)furan-2-yl)benzoic acid (100 mg, 0.177 mmol), sodium iodide (31.9 mg, 0.213 mmol) and 4-(2-chloroethyl)morpholine, Hydrochloride (66.0 mg, 0.355 mmol) in DMF (1 mL) was added TEA (0.124 mL, 0.887 mmol). The reaction mixture was stirred at 80° C. for 65 h. The reaction mixture was diluted with EtOAc and washed with water. The organic phase was washed with water 2× and the combined aqueous layers were washed with EtOAc. The combined organic layer was passed through a hydrophobic frit and concentrated. Purification by Si (0-10% MeOH/EtOAc) afforded the title compound as an orange solid. (72 mg, 60% yield). MS (m/z) 677.3 (M+H)⁺

### Intermediate 121: 2-(dimethylamino)ethyl 4-(5-((((R)-2-((R)-1-(N-(benzyloxy)formamido)propyl)heptanamido)methyl)carbamoyl)furan-2-yl)benzoate

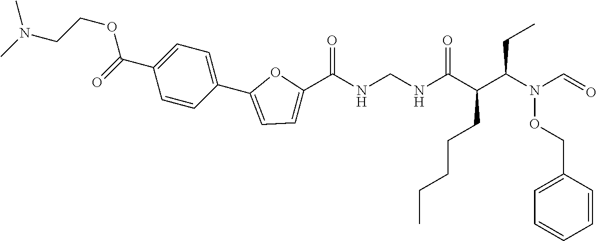 [SMILES: CCCCC[C@@H](C(=O)NCNC(=O)c1ccc(-c2ccc(C(=O)OCCN(C)C)cc2)o1)[C@@H](CC)N(C=O)OCc1ccccc1]

To a solution containing 4-(5-((((R)-2-((R)-1-(N-(benzyloxy)formamido)propyl)heptanamido)methyl)carbamoyl)furan-2-yl)benzoic acid (100 mg, 0.177 mmol), sodium iodide (31.9 mg, 0.213 mmol) and 2-chloro-N,N-dimethylethanamine, Hydrochloride (51.1 mg, 0.355 mmol) in DMF (1 mL) was added TEA (0.124 mL, 0.887 mmol). The reaction mixtures were stirred at 80° C. for 65 h. The reaction mixture was diluted with EtOAc and washed with water. The organic phase was washed with water 2× and the combined aqueous layers were washed with EtOAc. The combined organic layer was passed through a hydrophobic frit and concentrated. Purification by Si (0-20% MeOH/EtOAc) afforded the title compound as an orange solid. (19 mg, 17% yield). MS (m/z) 635.3 (M+H)⁺

### Intermediate 122: 2-(dimethylamino)-2-oxoethyl 4-(5-((((R)-2-((R)-1-(N-(benzyloxy)formamido)propyl)heptanamido)methyl)carbamoyl)furan-2-yl)benzoate

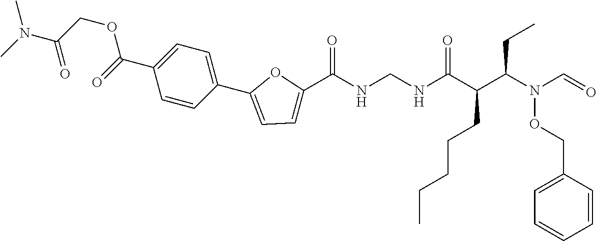 [SMILES: CCCCC[C@@H](C(=O)NCNC(=O)c1ccc(-c2ccc(C(=O)OCC(=O)N(C)C)cc2)o1)[C@@H](CC)N(C=O)OCc1ccccc1]

To a solution containing 4-(5-((((R)-2-((R)-1-(N-(benzyloxy)formamido)propyl)heptanamido)methyl)carbamoyl)furan-2-yl)benzoic acid (100 mg, 0.177 mmol), sodium iodide (31.9 mg, 0.213 mmol) and 2-chloro-N,N-dimethylacetamide (0.036 mL, 0.355 mmol) in DMF (1 mL) was added TEA (0.124 mL, 0.887 mmol). The reaction mixtures were stirred at RT for 65 h. Additional 2-chloro-N,N-dimethylacetamide (0.036 mL, 0.355 mmol), sodium iodide (31.9 mg, 0.213 mmol) and TEA (0.124 mL, 0.887 mmol) was added and stirred for 96 h RT.

The reaction mixture was diluted with EtOAc and washed with water. The organic phase was washed with water 2× and the combined aqueous layers were washed with EtOAc. The combined organic layer was passed through a hydrophobic frit and concentrated. Purification by Si (0-10% MeOH/EtOAc) afforded the title compound as an yellow solid. (86 mg, 75% yield). MS (m/z) 649.2 (M+H)⁺

### Intermediate 123: Di-tert-butyl ((4R,5R)-4-ethyl-3-formyl-6,10-dioxo-5-pentyl-10-(5-phenylfuran-2-yl)-2-oxa-3,7,9-triazadecyl) Phosphate

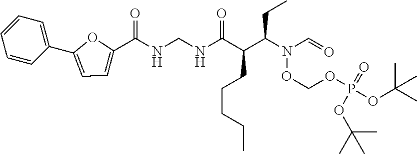 [SMILES: CCCCC[C@@H](C(=O)NCNC(=O)c1ccc(-c2ccccc2)o1)[C@@H](CC)N(C=O)OCOP(=O)(OC(C)(C)C)OC(C)(C)C]

DIPEA (305 μl, 1.746 mmol) was added to a stirring solution of N—(((R)-2-((R)-1-(N-hydroxyformamido)propyl)heptanamido)methyl)-5-phenylfuran-2-carboxamide (250 mg, 0.582 mmol) and di-tert-butyl (chloromethyl) phosphate (452 mg, 1.746 mmol) in THF (3880 μl). The resulting mixture was stirred for 24 h and evaporated. Purification by Si (0-100% EtOAc/Hex) afforded the title compound as an yellow gum. (320 mg, 84% yield). MS (m/z) 652.7 (M+H)⁺

### Intermediate 124: (S)-di-tert-butyl 2-(4-(5-((((R)-2-((R)-1-(N-((2-((di-tert-butoxyphosphoryl)oxy)-6-methylbenzoyl)oxy)formamido)propyl)heptanamido)methyl)carbamoyl)furan-2-yl)-2-ethoxybenzamido)succinate

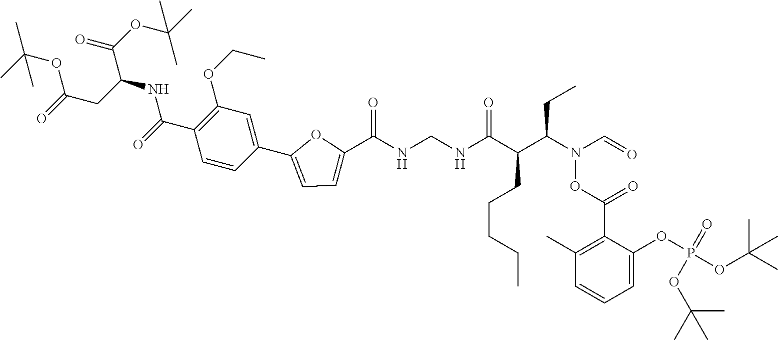 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(C=O)OC(=O)c1c(C)cccc1OP(=O)(OC(C)(C)C)OC(C)(C)C]

### Step 1: Allyl 2-((di-tert-butoxyphosphoryl)oxy)-6-methylbenzoate

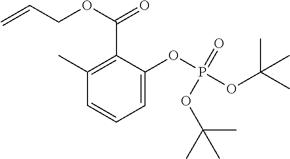 [SMILES: C=CCOC(=O)c1c(C)cccc1OP(=O)(OC(C)(C)C)OC(C)(C)C]

To a solution containing allyl 2-hydroxy-6-methylbenzoate (300 mg, 1.561 mmol) and tetrabutylammonium iodide (34.6 mg, 0.094 mmol) in DMF (10 mL) and added (carefully) sodium hydride (94 mg, 2.341 mmol). After 15 mins, di-tert-butyl phosphorochloridate (357 mg, 1.561 mmol) was added. The reaction was stirred for 90 min at 50° C. The reaction was diluted with water carefully and extracted with EtOAc (3×10 mL). The combined organic phase was washed with water and then passed through a hydrophobic frit and concentrated. Purification by Si (0-70% EtOAc/Hex) afforded the title compound as a yellow oil. (181 mg, 30% yield). MS (m/z) 272.9 (M (−2XtBu)+H)⁺

### Step 2: 2-((di-tert-butoxyphosphoryl)oxy)-6-methylbenzoic Acid

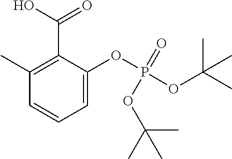 [SMILES: Cc1cccc(OP(=O)(OC(C)(C)C)OC(C)(C)C)c1C(=O)O]

To a solution containing allyl 2-((di-tert-butoxyphosphoryl)oxy)-6-methylbenzoate (181 mg, 0.471 mmol, 30.2% yield) in MeCN (5.00 mL) was added 1,3-dimethylpyrimidine-2,4,6(1H,3H,5H)-trione (402 mg, 2.58 mmol) and tetrakis(triphenylphosphine)palladium(0) (180 mg, 0.156 mmol) in a microwave vial. Degassed by bubbling nitrogen through the reaction mixture before sealing tube and letting stir at RT for 3 h. The reaction was filtered through a plug of celite, washed plug with MeCN (5 mL) and concentrated. Purification by Si (0-20% MeOH/EtOAc) afforded the title compound as a orange residue. (114 mg, 70% yield). MS (m/z) 345.0 (M+H)⁺

### Step 3: (S)-di-tert-butyl 2-(4-(5-((((R)-2-((R)-1-(N-((2-((di-tert-butoxyphosphoryl)oxy)-6-methylbenzoyl)oxy)formamido)propyl)heptanamido)methyl)carbamoyl)furan-2-yl)-2-ethoxybenzamido)succinate

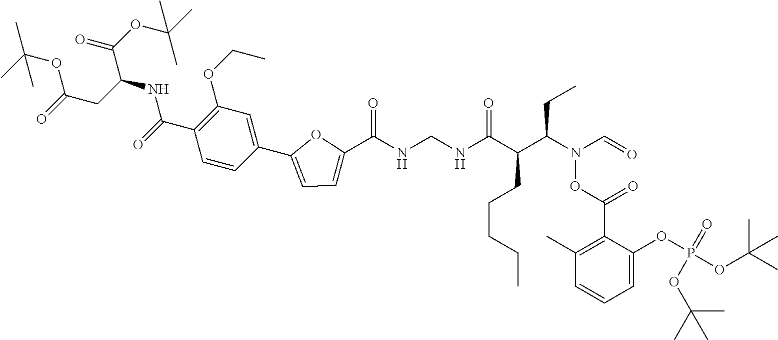 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(C=O)OC(=O)c1c(C)cccc1OP(=O)(OC(C)(C)C)OC(C)(C)C]

To a solution containing (S)-di-tert-butyl 2-(2-ethoxy-4-(5-((((R)-2-((R)-1-(N-hydroxyformamido)propyl)heptanamido)methyl)carbamoyl)furan-2-yl)benzamido)succinate (240 mg, 0.322 mmol) and 2-((di-tert-butoxyphosphoryl)oxy)-6-methylbenzoic acid (111 mg, 0.322 mmol) in MeCN (1.5 mL) was added DIPEA (0.169 mL, 0.967 mmol) and added HATU (147 mg, 0.387 mmol). The reaction was stirred at RT for 2 h and concentrated. Purification by Si (0-100% EtOAc/Hex followed by 100% MeOH) afforded the title compound as a orange residue. (130 mg, 38% yield). MS (m/z) 959.5 (M-(2XtBu)+H)⁺

### Intermediate 125: (S)-di-tert-butyl 2-(2-ethoxy-4-(5-((((R)-2-((R)-1-(N-hydroxyformamido)propyl)heptanamido)methyl)carbamoyl)furan-2-yl)benzamido)succinate

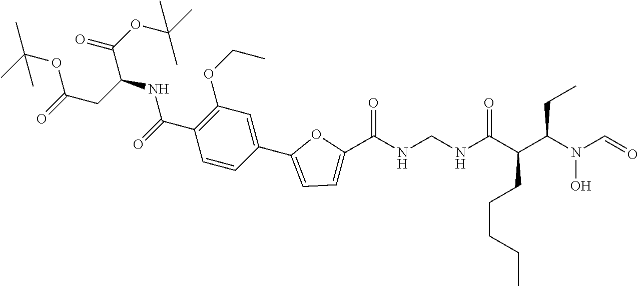 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(O)C=O]

### Step 1: (S)-di-tert-butyl 2-(4-bromo-2-ethoxybenzamido)succinate

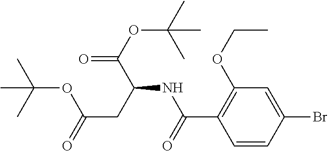 [SMILES: CCOc1cc(Br)ccc1C(=O)N[C@@H](CC(=O)OC(C)(C)C)C(=O)OC(C)(C)C]

To a solution containing (S)-di-tert-butyl 2-aminosuccinate, Hydrochloride (6.90 g, 24.48 mmol) and 4-bromo-2-ethoxybenzoic acid (5 g, 20.40 mmol) in DCM (40 mL) at RT was added HATU (9.31 g, 24.48 mmol) followed by DIPEA (10.69 mL, 61.2 mmol). The reaction mixture was stirred at RT for 2 h. The reaction mixture was diluted with NH₄Cl aq. and extracted with DCM. The combined organic layer was dried over MgSO4, filtered and concentrated onto Celite. Purification by Si (0-100% EtOAc/Hex) afforded the title compound as a pale yellow oil. (5.2 g, 52% yield). MS (m/z) 472.2 (M)⁺

### Step 2: (S)-di-tert-butyl 2-(2-ethoxy-4-(4,4,5,5-tetramethyl-1,3,2-dioxaborolan-2-yl)benzamido)succinate

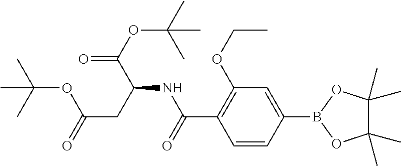 [SMILES: CCOc1cc(B2OC(C)(C)C(C)(C)O2)ccc1C(=O)N[C@@H](CC(=O)OC(C)(C)C)C(=O)OC(C)(C)C]

(S)-di-tert-butyl 2-(4-bromo-2-ethoxybenzamido)succinate (5.23 g, 11.07 mmol) in 1,4-Dioxane (50 mL) was degassed for 5 min with N2. At this time, 4,4,4′,4′,5,5,5′,5′-octamethyl-2,2′-bi(1,3,2-dioxaborolane), potassium acetate (3.26 g, 33.2 mmol), and PdCl2(dppf)-CH2Cl2 (0.814 g, 0.996 mmol) were added and degassed for 5 min with N2 and then heated to 90° C. for 6 h under N2. The reaction was cooled and diluted with EtOAc and brine and filtered through celite and washed with EtOAc. Organic phase was dried over MgSO4, filtered and concentrated onto SiO2. Purification by Si (0-50% EtOAc/Hex) afforded the title compound as a brown oil. (5.3 g, 93% yield). MS (m/z) 520.4 (M+H)⁺

### Step 3: (S)-5-(4-((1,4-di-tert-butoxy-1,4-dioxobutan-2-yl)carbamoyl)-3-ethoxyphenyl) furan-2-carboxylic Acid

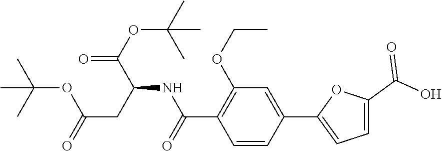 [SMILES: CCOc1cc(-c2ccc(C(=O)O)o2)ccc1C(=O)N[C@@H](CC(=O)OC(C)(C)C)C(=O)OC(C)(C)C]

N₂ was bubbled through a solution of (S)-di-tert-butyl 2-(2-ethoxy-4-(4,4,5,5-tetramethyl-1,3,2-dioxaborolan-2-yl)benzamido)succinate (5.33 g, 10.26 mmol) in 1,4-Dioxane (68.4 mL) for 30 min. 5-bromofuran-2-carboxylic acid (2.450 g, 12.83 mmol) and PdCl2 (dppf) DCM (0.461 g, 0.564 mmol) were added followed by addition of sodium carbonate (1 M) (30.8 mL, 30.8 mmol). The resulting mixture was heated to 50° C. under N₂ for 30 min and cooled to RT. The pH of the crude reaction mixture was adjusted to 4 by careful addition of 6N HCl. EtOAc (500 mL) was added and the organic phase was washed with water (2×) and brine (1×). The combined washes were extracted using EtOAc (1×). The combined organics were dried over MgSO4, filtered, and concentrated. Purification by Si (0-100% EtOAc/Hex) afforded the title compound as an orange solid. (3.5 g, 62% yield). MS (m/z) 504.1 (M+H)⁺

### Step 4: (S)-di-tert-butyl 2-(4-(5-((((R)-2-((R)-1-(N-(benzyloxy)formamido)propyl)heptanamido)methyl)carbamoyl)furan-2-yl)-2-ethoxybenzamido)succinate

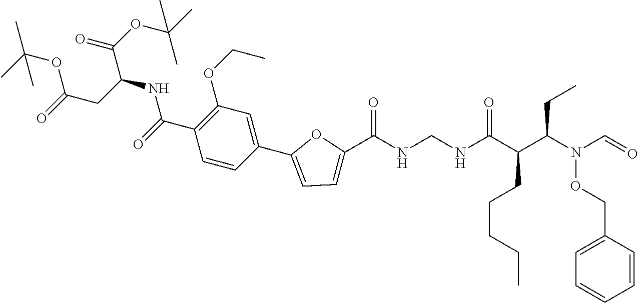 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(C=O)OCc1ccccc1]

A solution of (R)—N-(aminomethyl)-2-((R)-1-(N-(benzyloxy)formamido)propyl)heptanamide (1.035 g, 2.96 mmol) in DMF (7.05 mL) was prepared. A solution of (S)-5-(4-((1,4-di-tert-butoxy-1,4-dioxobutan-2-yl)carbamoyl)-3-ethoxyphenyl)furan-2-carboxylic acid (1.42 g, 2.82 mmol), HATU (1.287 g, 3.38 mmol), and DIPEA (1.478 mL, 8.46 mmol) in MeCN (7.05 mL) was stirred for 30 min at RT, and was then slowly added to the amine solution. The mixture was stirred for 1 h at RT. EtOAc was added, and the organics were washed with saturated NaHCO₃ (2×) and brine (1×). The combined washes were back extracted using EtOAc (1×). The combined extracts were dried over MgSO4, filtered, and concentrated. Purification by Si (0-100% EtOAc/Hex) afforded the title compound as a colorless solid. (1.58 g, 66% yield). MS (m/z) 835.3 (M+H)⁺

### Step 5: (S)-di-tert-butyl 2-(2-ethoxy-4-(5-((((R)-2-((R)-1-(N-hydroxyformamido)propyl)heptanamido)methyl)carbamoyl)furan-2-yl)benzamido)succinate

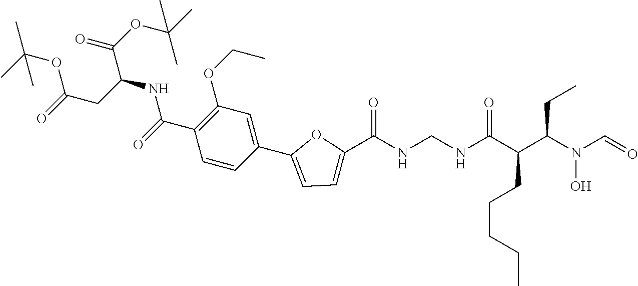 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(O)C=O]

Pd—C (10% Degussa Type) (0.169 g, 0.159 mmol) was added to a solution of (S)-di-tert-butyl 2-(4-(5-((((R)-2-((R)-1-(N-(benzyloxy)formamido)propyl)heptanamido)methyl)carbamoyl)furan-2-yl)-2-ethoxybenzamido)succinate (1.327 g, 1.589 mmol) in DCM (2.65 mL). MeOH (13.24 mL) was added under N₂. Ammonium formate (1.002 g, 15.89 mmol) was added, and a N₂ line was affixed to flask. The mixture was stirred for 2 h at which time it was placed under a hydrogen balloon for 20 min. The mixture was evacuated and flushed with N₂ (3×) and was filtered through a pad of Celite and concentrated. Purification by Si (0-100% EtOAc/Hex and then 3% MeOH/EtOAc) afforded the title compound as a grey solid. (1.1 g, 90% yield). MS (m/z) 745.4 (M+H)⁺

### Intermediate 126: Tert-butyl 4-(5-((((R)-2-((R)-1-(N-(benzoyloxy)formamido)propyl)heptanamido)methyl)carbamoyl)furan-2-yl)benzoate

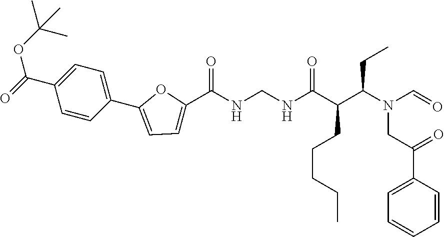 [SMILES: CCCCC[C@@H](C(=O)NCNC(=O)c1ccc(-c2ccc(C(=O)OC(C)(C)C)cc2)o1)[C@@H](CC)N(C=O)CC(=O)c1ccccc1]

### Step 1: Tert-butyl 4-(5-((((R)-2-((R)-1-(N-(benzyloxy)formamido)propyl)heptanamido)methyl)carbamoyl)furan-2-yl)benzoate

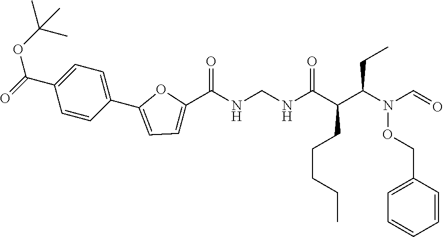 [SMILES: CCCCC[C@@H](C(=O)NCNC(=O)c1ccc(-c2ccc(C(=O)OC(C)(C)C)cc2)o1)[C@@H](CC)N(C=O)OCc1ccccc1]

In a microwave vial, to a solution containing N—(((R)-2-((R)-1-(N-(benzyloxy)formamido)propyl)heptanamido)methyl)-5-bromofuran-2-carboxamide (250 mg, 0.479 mmol) and (4-(tert-butoxycarbonyl)phenyl)boronic acid (117 mg, 0.526 mmol) in 1,4-dioxane (2.8 mL) and added Na2CO3 (1M in water) (1.436 mL, 1.436 mmol) and PdCl2(dppf)-CH2Cl2 (39.1 mg, 0.048 mmol). The vial was sealed and heated to 100° C. for 6 mins in a microwave reactor. Reaction was diluted with EtOAc (10 mL) and brine (10 mL), pass through a 0.45 uM PTFE filter and separate layers. The organic phase was passed through a hydrophobic frit and concentrated. Purification by Si (0-100% EtOAc/Hex) afforded the title compound as an orange oil. (316 mg, ˜100% yield). MS (m/z) 620.5 (M+H)⁺

### Step 2: Tert-butyl 4-(5-((((R)-2-((R)-1-(N-hydroxyformamido)propyl)heptanamido)methyl)carbamoyl)furan-2-yl)benzoate

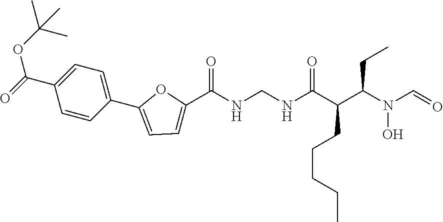 [SMILES: CCCCC[C@@H](C(=O)NCNC(=O)c1ccc(-c2ccc(C(=O)OC(C)(C)C)cc2)o1)[C@@H](CC)N(O)C=O]

To a solution containing Pd/C (50.9 mg, 0.048 mmol) in DCM (0.700 mL) was added tert-butyl 4-(5-((((R)-2-((R)-1-(N-(benzyloxy)formamido)propyl)heptanamido)methyl)carbamoyl)furan-2-yl)benzoate (315.8 mg, 0.510 mmol, 106% yield) in MeOH (2.80 mL). Ammonium formate (151 mg, 2.393 mmol) was added and stirred for 25 min. The reaction mixture was passed through a pad of celite and wash with MeOH (10 mL) and the filtrate was concentrated. Purification by Si (0-100% EtOAc/Hex) afforded the title compound as a yellow oil. (229 mg, 90% yield). MS (m/z) 530.2 (M+H)⁺

### Step 3: Tert-butyl 4-(5-((((R)-2-((R)-1-(N-(benzoyloxy)formamido)propyl)heptanamido)methyl)carbamoyl)furan-2-yl)benzoate

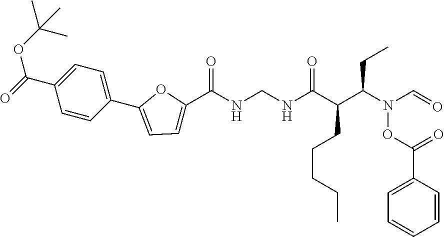 [SMILES: CCCCC[C@@H](C(=O)NCNC(=O)c1ccc(-c2ccc(C(=O)OC(C)(C)C)cc2)o1)[C@@H](CC)N(C=O)OC(=O)c1ccccc1]

To a solution containing tert-butyl 4-(5-((((R)-2-((R)-1-(N-hydroxyformamido)propyl)heptanamido)methyl)carbamoyl)furan-2-yl)benzoate (225 mg, 0.425 mmol) in THF (2.5 mL) was added DIPEA (0.223 mL, 1.274 mmol) and benzoyl chloride (0.099 mL, 0.850 mmol). The reaction was stirred for 3 h and concentrated. Purification by Si (0-100% EtOAc/Hex) afforded the title compound as a yellow residue. (238 mg, 88% yield). MS (m/z) 634.4 (M+H)⁺

### Intermediate 127: (S)-di-tert-butyl 2-(2-ethoxy-4-(5-((((R)-2-((R)-1-(N-((phenylcarbamoyl)oxy)formamido)propyl)heptanamido)methyl)carbamoyl)furan-2-yl)benzamido)succinate

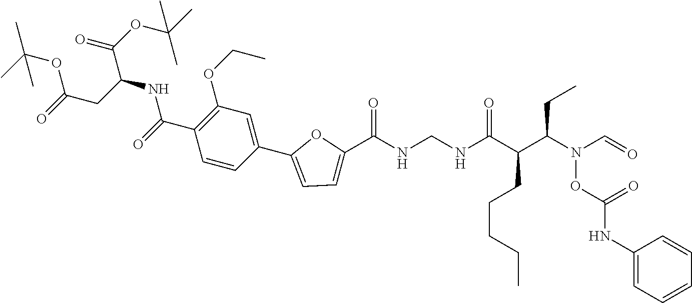 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(C=O)OC(=O)Nc1ccccc1]

To a solution containing triphosgene (0.044 g, 0.149 mmol) in DCM (2.014 mL) at 0° C. was added aniline (0.037 mL, 0.403 mmol) and TEA (0.123 mL, 0.886 mmol) in DCM (2.014 mL) drop wise. The mixture was stirred at RT for 15 min and (S)-di-tert-butyl 2-(2-ethoxy-4-(5-((((R)-2-((R)-1-(N-hydroxyformamido)propyl)heptanamido)methyl)carbamoyl)furan-2-yl)benzamido)succinate (0.15 g, 0.201 mmol) in DCM (2.014 mL) was added at 0° C. The solution was stirred at 0° C. for 10 min. The reaction mixture was allowed to warm to RT and stir for 18 h and concentrated. Purification by Si (0-100% EtOAc/Hex) afforded the title compound as a yellow oil. (80 mg, 23% yield). MS (m/z) 864.1 (M+H)⁺

### Intermediate 128: (S)-4-benzyl 1-((isobutyryloxy)methyl) 2-(4-(5-((((R)-2-((R)-1-(N-(benzyloxy)formamido)propyl)heptanamido)methyl)carbamoyl)furan-2-yl)-2-ethoxybenzamido)succinate

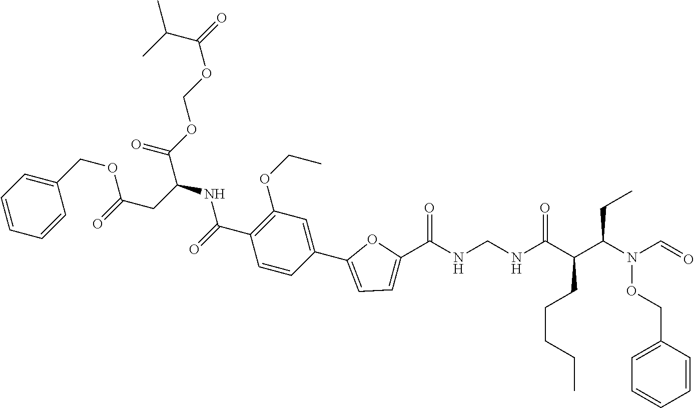 [SMILES: CCCCC[C@@H](C(=O)NCNC(=O)c1ccc(-c2ccc(C(=O)N[C@@H](CC(=O)OCc3ccccc3)C(=O)OCOC(=O)C(C)C)c(OCC)c2)o1)[C@@H](CC)N(C=O)OCc1ccccc1]

### Step 1: (S)-4-(benzyloxy)-2-(2-ethoxy-4-(4,4,5,5-tetramethyl-1,3,2-dioxaborolan-2-yl)benzamido)-4-oxobutanoic Acid

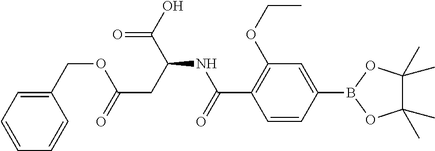 [SMILES: CCOc1cc(B2OC(C)(C)C(C)(C)O2)ccc1C(=O)N[C@@H](CC(=O)OCc1ccccc1)C(=O)O]

To a N₂ degassed solution containing (S)-dibenzyl 2-(4-bromo-2-ethoxybenzamido)succinate (110.42 g, 204 mmol) in 1,4-Dioxane (681 mL) was added 4,4,4′,4′,5,5,5′,5′-octamethyl-2,2′-bi(1,3,2-dioxaborolane) (62.3 g, 245 mmol), potassium acetate (60.2 g, 613 mmol), and PdCl2(dppf)-CH2Cl2 (16.69 g, 20.43 mmol). The resulting mixture was heated to 90° C. for 6 h. Additional amounts of each reagent were added (4,4,4′,4′,5,5,5′,5′-octamethyl-2,2′-bi(1,3,2-dioxaborolane) (15 g), potassium acetate (15 g), and PdCl2(dppf)-CH2Cl2 (3 g)) and the reaction was allowed to stir at 90° C. for 4 h more. The mixture was cooled to RT and stirred over night. Water was added to the cooled reaction, and the crude mixture was filtered through a pad of Celite. The Celite was washed thoroughly with excess EtOAc (1 L). The layers were separated and additional organics were extracted from the aqueous layer with EtOAc (2×1 L). The combined organics were washed with brine (3×), dried over MgSO₄, filtered, and concentrated. Purification by Si (0-75% EtOAc/Hex) afforded the title compound as an orange oil. (54 g, 53% yield). MS (m/z) 498.3 (M+H)⁺

### Step 2: (S)-4-(benzyloxy)-2-(4-(5-((((R)-2-((R)-1-(N-(benzyloxy)formamido)propyl)heptanamido)methyl)carbamoyl)furan-2-yl)-2-ethoxybenzamido)-4-oxobutanoic Acid

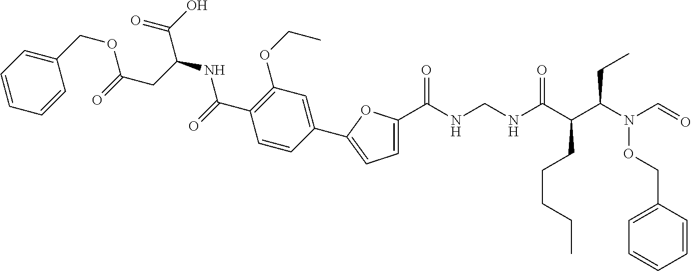 [SMILES: CCCCC[C@@H](C(=O)NCNC(=O)c1ccc(-c2ccc(C(=O)N[C@@H](CC(=O)OCc3ccccc3)C(=O)O)c(OCC)c2)o1)[C@@H](CC)N(C=O)OCc1ccccc1]

To a solution containing (S)-4-(benzyloxy)-2-(2-ethoxy-4-(4,4,5,5-tetramethyl-1,3,2-dioxaborolan-2-yl)benzamido)-4-oxobutanoic acid (1.714 g, 3.45 mmol) in 1,4-Dioxane (7.66 mL):2M Na2CO3 (7.66 mL) was added N—(((R)-2-((R)-1-(N-(benzyloxy)formamido)propyl)heptanamido)methyl)-5-bromofuran-2-carboxamide (1.2 g, 2.297 mmol), and PdCl2(dppf)-CH2Cl2 (0.188 g, 0.230 mmol). The reaction was heated to 50° C. for 1 h under Nitrogen. The reaction was poured into water and extracted into EtOAc (3×100 mL). The combined organic layers were washed with brine, dried over sodium sulfate and decolorizing C, filtered through a pad of celite and washed with EtOAc 3×. The filtrate was concentrated in-vacuo, dissolved in water (200 mL) and treated with 1N HCl (20 mL) upon which product precipitated out. The solution was stirred for 1 h at RT at which time it was filtered, washed with hexanes 2× and dried via suction filtration overnight to afford the title compounds as as a colorless solid. (1.58 g, 85% yield). MS (m/z) 813.6 (M+H)⁺

### Step 3: (S)-4-benzyl 1-((isobutyryloxy)methyl) 2-(4-(5-((((R)-2-((R)-1-(N-(benzyloxy)formamido)propyl)heptanamido)methyl)carbamoyl)furan-2-yl)-2-ethoxybenzamido)succinate

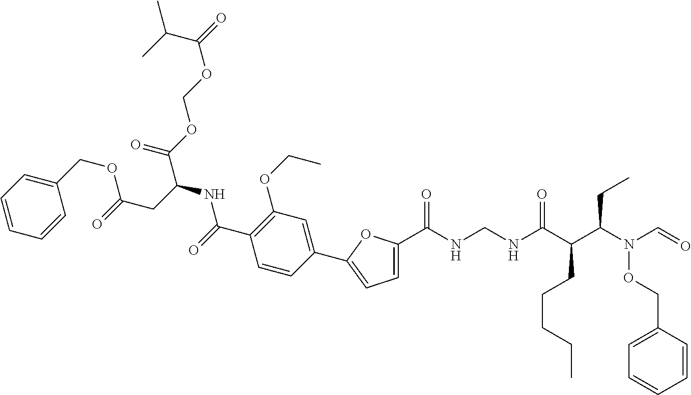 [SMILES: CCCCC[C@@H](C(=O)NCNC(=O)c1ccc(-c2ccc(C(=O)N[C@@H](CC(=O)OCc3ccccc3)C(=O)OCOC(=O)C(C)C)c(OCC)c2)o1)[C@@H](CC)N(C=O)OCc1ccccc1]

To a solution containing (S)-4-(benzyloxy)-2-(4-(5-((((R)-2-((R)-1-(N-(benzyloxy)formamido)propyl)heptanamido)methyl)carbamoyl)furan-2-yl)-2-ethoxybenzamido)-4-oxobutanoic acid (200 mg, 0.246 mmol) in DMF (2.5 mL) was added sodium carbonate (104 mg, 0.984 mmol) followed by chloromethyl isobutyrate (124 μl, 0.984 mmol). The reaction was stirred at RT for 18 h in a sealed tube. Purification by Si (0-100% EtOAc/Hex) afforded the title compound as an pale yellow oil. (198 mg, 88% yield). MS (m/z) 914.7 (M+H)⁺

### Intermediate 129: (((3-(5-((((R)-2-((R)-1-(N-(benzyloxy)formamido)propyl)heptanamido)methyl)carbamoyl)furan-2-yl)-5-ethoxyphenyl)phosphoryl)bis(oxy))bis(methylene) Diisopropyl Dicarbonate

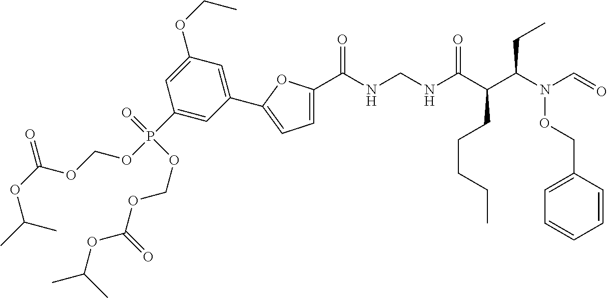 [SMILES: CCCCC[C@@H](C(=O)NCNC(=O)c1ccc(-c2cc(OCC)cc(P(=O)(OCOC(=O)OC(C)C)OCOC(=O)OC(C)C)c2)o1)[C@@H](CC)N(C=O)OCc1ccccc1]

### Step 1: Dimethyl (3-ethoxy-5-(4,4,5,5-tetramethyl-1,3,2-dioxaborolan-2-yl)phenyl)phosphonate

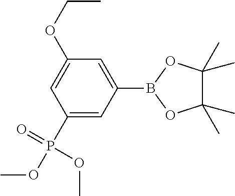

A solution containing dimethyl (3-bromo-5-ethoxyphenyl)phosphonate (12.6 g, 40.8 mmol) in 1,4-Dioxane (163 mL) was degassed by bubbling N₂ through it for 10 min. 4,4,4′,4′,5,5,5′,5′-octamethyl-2,2′-bi(1,3,2-dioxaborolane) (11.39 g, 44.8 mmol), PdCl2(dppf)-CH2Cl2 (3.00 g, 3.67 mmol), and potassium acetate (12.00 g, 122 mmol) were added, and the mixture was degassed further for 5 min. The mixture was then heated to 90° C. for 12 h. Water was added, and the mixture was filtered through Celite washing with EtOAc. The phases were separated and the organics were extracted using EtOAc (2×). The combined organics were washed with brine, dried over MgSO4, filtered, and concentrated onto silica. Purification by Si (0-100% EtOAc/Hex) afforded the title compound as an orange oil. (16.4 g, ˜100% yield). MS (m/z) 357.1 (M+H)⁺

### Step 2: Tert-butyl 5-(3-(dimethoxyphosphoryl)-5-ethoxyphenyl)furan-2-carboxylate

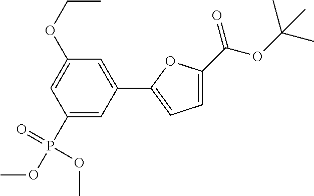 [SMILES: CCOc1cc(-c2ccc(C(=O)OC(C)(C)C)o2)cc(P(=O)(OC)OC)c1]

A solution of dimethyl (3-ethoxy-5-(4,4,5,5-tetramethyl-1,3,2-dioxaborolan-2-yl)phenyl)phosphonate (16.38 g, 46.0 mmol) in 1,4-Dioxane (184 mL) was degassed by bubbling N₂ through it for 10 min. Tert-butyl 5-bromofuran-2-carboxylate (12.50 g, 50.6 mmol), PdCl2(dppf)-CH2Cl2 (2.066 g, 2.53 mmol), and sodium carbonate (138 mL, 138 mmol) were added, and the mixture was degassed further for 5 min. The mixture was then heated to 50° C. for 30 min. Water was added, and the mixture was extracted with EtOAc (3×). The combined extracts were washed with brine (3×), dried over MgSO4, filtered, and concentrated. Purification by Si (0-100% EtOAc/Hex) afforded the title compound as a brown oil. (16.5 g, 85% yield). MS (m/z) 397.1 (M+H)⁺

### Step 3: 5-(3-(dimethoxyphosphoryl)-5-ethoxyphenyl)furan-2-carboxylic Acid

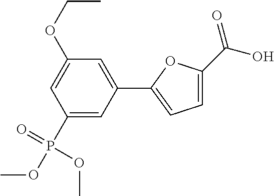 [SMILES: CCOc1cc(-c2ccc(C(=O)O)o2)cc(P(=O)(OC)OC)c1]

To a stirring 0° C. solution of tert-butyl 5-(3-(dimethoxyphosphoryl)-5-ethoxyphenyl)furan-2-carboxylate (16.23 g, 40.9 mmol) in DCM (512 mL) was slowly added TFA (95 mL, 1228 mmol). The resulting mixture was allowed to warm to RT over 2 h, and was stirred at RT for 2 h. The mixture was cooled back down to 0° C. Saturated aqueous NaHCO₃ was added slowly until the pH of the reaction mixture was above 8. The DCM was evaporated, and the organics were extracted using EtOAc (2×). The combined extracts were washed with brine, dried over MgSO₄, filtered, and concentrated to dryness. The solids were taken up in ether and stirred for 5 min and filtered and dried to afford the title compound as a brown/grey solid. (9.9 g, 72% yield). MS (m/z) 340.9 (M+H)⁺

### Step 4: Dimethyl (3-(5-((((R)-2-((R)-1-(N-(benzyloxy)formamido)propyl)heptanamido)methyl)carbamoyl)furan-2-yl)-5-ethoxyphenyl)phosphonate

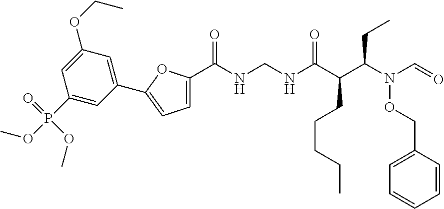 [SMILES: CCCCC[C@@H](C(=O)NCNC(=O)c1ccc(-c2cc(OCC)cc(P(=O)(OC)OC)c2)o1)[C@@H](CC)N(C=O)OCc1ccccc1]

To a solution containing 5-(3-(dimethoxyphosphoryl)-5-ethoxyphenyl)furan-2-carboxylic acid (0.544 g, 1.6 mmol) in MeCN (8.00 mL) was added HATU (0.669 g, 1.760 mmol), DIPEA (1.118 mL, 6.40 mmol), and (R)—N-(aminomethyl)-2-((R)-1-(N-(benzyloxy)formamido)propyl)heptanamide (0.56 g, 1.602 mmol) in was stirred at RT overnight. Water was added and the reaction was extracted with EtOAc, dried over Na2SO4 and concentrated. Purification by Si (0-100% EtOAc/Hex) afforded the title compound as a brown oil. (0.5 g, 47% yield). MS (m/z) 672.3 (M+H)⁺

### Step 5: (3-(5-((((R)-2-((R)-1-(N-(benzyloxy)formamido)propyl)heptanamido)methyl)carbamoyl)furan-2-yl)-5-ethoxyphenyl)phosphonic Acid

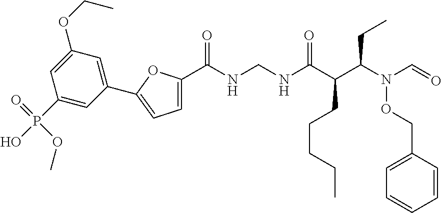 [SMILES: CCCCC[C@@H](C(=O)NCNC(=O)c1ccc(-c2cc(OCC)cc(P(=O)(O)OC)c2)o1)[C@@H](CC)N(C=O)OCc1ccccc1]

To a solution containing dimethyl (3-(5-((((R)-2-((R)-1-(N-(benzyloxy)formamido)propyl)heptanamido)methyl)carbamoyl)furan-2-yl)-5-ethoxyphenyl)phosphonate (0.5 g, 0.744 mmol) in MeCN (1.489 mL) and DCM (5.95 mL) at 0° C. under N₂ was added bromotrimethylsilane (0.483 mL, 3.72 mmol). The reaction was stirred at 0° C. for 5 mins and then at RT for 1 hr. The reaction was cooled to 0° C. again and quenched with 1N HCl (˜5 mL). The reaction was then extracted with EtOAc, dried over Na₂SO₄ and concentrated to give the title compound as a light yellow, crispy foam. (0.4 g, 83% yield). MS (m/z) 644.3 (M+H)⁺

### Step 6: (((3-(5-((((R)-2-((R)-1-(N-(benzyloxy)formamido)propyl)heptanamido)methyl)carbamoyl)furan-2-yl)-5-ethoxyphenyl)phosphoryl)bis(oxy))bis(methylene) Diisopropyl Dicarbonate

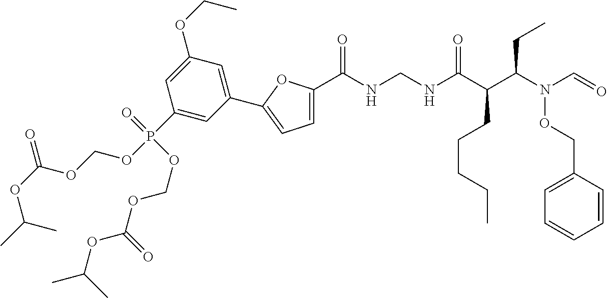 [SMILES: CCCCC[C@@H](C(=O)NCNC(=O)c1ccc(-c2cc(OCC)cc(P(=O)(OCOC(=O)OC(C)C)OCOC(=O)OC(C)C)c2)o1)[C@@H](CC)N(C=O)OCc1ccccc1]

To a solution containing (3-(5-((((R)-2-((R)-1-(N-(benzyloxy)formamido)propyl)heptanamido)methyl)carbamoyl)furan-2-yl)-5-ethoxyphenyl)phosphonic acid (0.125 g, 0.194 mmol) in DMF (1 mL) was added chloromethyl isopropyl carbonate (0.103 mL, 0.777 mmol) and potassium carbonate (0.107 g, 0.777 mmol). The reaction was stirred at 60° C. overnight. The reaction was cooled, water was added and the reaction was extracted with EtOAc. The organic layer was washed with water, brine, dried over Na₂SO₄ and concentrated. Purification by Si (0-100% EtOAc/Hex) afforded the title compound as a clear foam. (40 mg, 24% yield). MS (m/z) 876.3 (M+H)⁺

### Intermediate 130: Diphenyl (3-(5-((((R)-2-((R)-1-(N-(benzyloxy)formamido)propyl)heptanamido)methyl)carbamoyl)furan-2-yl)-5-ethoxyphenyl)phosphonate

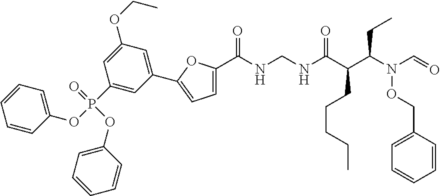 [SMILES: CCCCC[C@@H](C(=O)NCNC(=O)c1ccc(-c2cc(OCC)cc(P(=O)(Oc3ccccc3)Oc3ccccc3)c2)o1)[C@@H](CC)N(C=O)OCc1ccccc1]

To a solution containing (3-(5-((((R)-2-((R)-1-(N-(benzyloxy)formamido)propyl)heptanamido)methyl)carbamoyl)furan-2-yl)-5-ethoxyphenyl)phosphonic acid (0.1 g, 0.155 mmol) in DCM (1.554 mL) was added oxalyl chloride (0.030 mL, 0.342 mmol) and DMF (2 drops). The reaction was stirred at RT for 2 h. The reaction was concentrated to a crispy foam. Phenol (0.029 g, 0.311 mmol) was added to the foam and the reaction was dissolved in DCM (1.5 mL) and cooled in an ice bath. TEA (0.043 mL, 0.311 mmol) was added slowly and the reaction was allowed to warm up to RT overnight. Water and DCM was added and the layers were separated. The organic layer was passed through a hydrophobic cartridge and concentrated. Purification by Si (0-100% EtOAc/Hex) afforded the title compound as a clear foam. (13 mg, 11% yield). MS (m/z) 796.4 (M+H)⁺

### Intermediate 131: (((3-(5-((((R)-2-((R)-1-(N-(benzyloxy)formamido)propyl)heptanamido)methyl)carbamoyl)furan-2-yl)-5-ethoxyphenyl)phosphoryl)bis(oxy))bis(methylene) Bis(2-methylpropanoate)

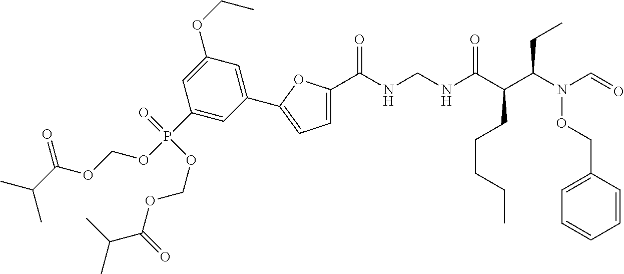 [SMILES: CCCCC[C@@H](C(=O)NCNC(=O)c1ccc(-c2cc(OCC)cc(P(=O)(OCOC(=O)C(C)C)OCOC(=O)C(C)C)c2)o1)[C@@H](CC)N(C=O)OCc1ccccc1]

To a solution containing (3-(5-((((R)-2-((R)-1-(N-(benzyloxy)formamido)propyl)heptanamido)methyl)carbamoyl)furan-2-yl)-5-ethoxyphenyl)phosphonic acid (0.175 g, 0.272 mmol) in DMF (1.4 mL) was added chloromethyl isobutyrate (0.076 mL, 0.598 mmol), and potassium carbonate (0.083 g, 0.598 mmol). The reaction was stirred at 60° C. overnight. Chloromethyl isobutyrate (0.076 mL, 0.598 mmol) and potassium carbonate (0.083 g, 0.598 mmol) were added and the reaction was stirred at 60° C. for 24 h and then at RT for 48 h. Water was added and the reaction was extracted with EtOAc. The organic layer was washed with brine, dried over Na₂SO₄ and concentrated. Purification by Si (0-70% EtOAc/Hex) afforded the title compound as a clear foam. (55 mg, 10% yield). MS (m/z) 844.4 (M+H)⁺

### Intermediate 132: (S)-4-benzyl 1-phenyl 2-(4-(5-((((R)-2-((R)-1-(N-(benzyloxy)formamido)propyl)heptanamido)methyl)carbamoyl)furan-2-yl)-2-ethoxybenzamido)succinate

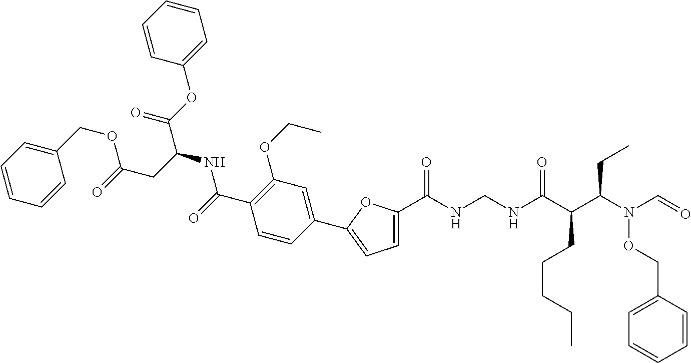 [SMILES: CCCCC[C@@H](C(=O)NCNC(=O)c1ccc(-c2ccc(C(=O)N[C@@H](CC(=O)OCc3ccccc3)C(=O)Oc3ccccc3)c(OCC)c2)o1)[C@@H](CC)N(C=O)OCc1ccccc1]

To a cooled solution of (S)-4-(benzyloxy)-2-(4-(5-((((R)-2-((R)-1-(N-(benzyloxy)formamido)propyl)heptanamido)methyl)carbamoyl)furan-2-yl)-2-ethoxybenzamido)-4-oxobutanoic acid (120 mg, 0.148 mmol) in DCM (1.5 mL) was added methanesulfonyl chloride (12.65 μl, 0.162 mmol) and 1-methylimidazole (23.53 μl, 0.295 mmol). Reaction stirred for 30 min before the addition of phenol (13.9 mg, 0.15 mmol). The reaction was allowed to warm to RT overnight. Purification by Si (0-100% EtOAc/Hex) afforded the title compound as a colorless solid. (45 mg, 34% yield). MS (m/z) 889.6 (M+H)⁺

### Intermediate 133: (S)-bis((isobutyryloxy)methyl) 2-(4-(5-((((R)-2-((R)-1-(N-(benzyloxy)formamido)propyl)heptanamido)methyl)carbamoyl)furan-2-yl)-2-ethoxybenzamido)succinate

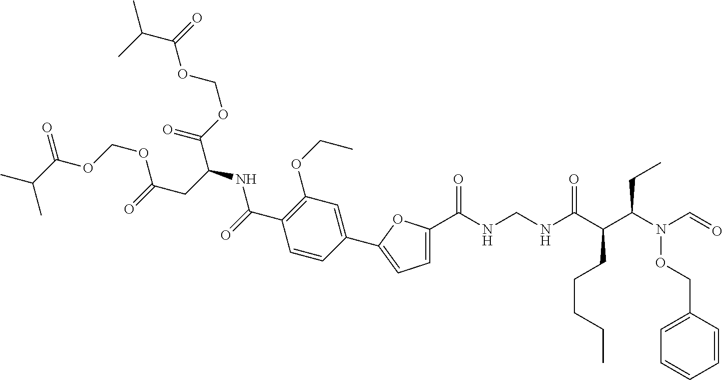 [SMILES: CCCCC[C@@H](C(=O)NCNC(=O)c1ccc(-c2ccc(C(=O)N[C@@H](CC(=O)OCOC(=O)C(C)C)C(=O)OCOC(=O)C(C)C)c(OCC)c2)o1)[C@@H](CC)N(C=O)OCc1ccccc1]

### Step 1: (S)-dimethyl 2-(4-bromo-2-ethoxybenzamido)succinate

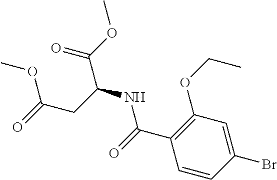 [SMILES: CCOc1cc(Br)ccc1C(=O)N[C@@H](CC(=O)OC)C(=O)OC]

To a suspension containing 4-bromo-2-ethoxybenzoic acid (43.27 g, 177 mmol), (S)-dimethyl 2-aminosuccinate, Hydrochloride (36.6 g, 185 mmol), and TEA (73.8 mL, 530 mmol) in DCM (420 mL) (placed in a water bath to avoid warming) was added T3P (50% wt in EtOAc) (150 mL, 252 mmol) drop wise (in 1 h 10 mins). After addition was completed, the reaction was stirred at RT for 2 h. The reaction was diluted with DCM, washed with water, 1N HCl, and then with sat. NaHCO3. The organic layer was dried over MgSO4 and concentrated. Purification by Si (0-50% EtOAc/Hex) afforded the title compound as a yellow oil. (62.7 g, 91% yield). MS (m/z) 388.0 (M)+/390.0 (M+2H)

### Step 2: (S)-dimethyl 2-(2-ethoxy-4-(4,4,5,5-tetramethyl-1,3,2-dioxaborolan-2-yl)benzamido)succinate

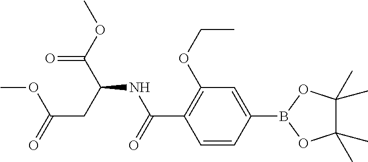

A mixture of (S)-dimethyl 2-(4-bromo-2-ethoxybenzamido)succinate (31.93 g, 82 mmol), bis(pinacolato)diboron (25.06 g, 99 mmol), potassium acetate (33 g, 336 mmol), and PdCl2(dppf)-CH2Cl2 (2.69 g, 3.29 mmol) in 1,4-Dioxane (300 mL) was stirred at 80° C. for 10 h. The reaction was cooled, hexane (˜800 mL) was added the reaction was filtered through a pad of silica gel. The silica gel was washed with EtOAc/hex (1/3) until all the product out. The filtrate was concentrated to afford the title compound as a brown oil. (148 g, 89% yield). MS (m/z) 436.2 (M+H)⁺

### Step 3: (S)-2-(2-ethoxy-4-(4,4,5,5-tetramethyl-1,3,2-dioxaborolan-2-yl)benzamido)succinic Acid

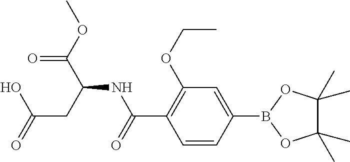 [SMILES: CCOc1cc(B2OC(C)(C)C(C)(C)O2)ccc1C(=O)N[C@@H](CC(=O)O)C(=O)OC]

To a solution containing (S)-dimethyl 2-(2-ethoxy-4-(4,4,5,5-tetramethyl-1,3,2-dioxaborolan-2-yl)benzamido)succinate (1.88 g, 4.32 mmol) THF (32.4 mL):Water (10.80 mL) was added LiOH (0.310 g, 12.96 mmol). The reaction was stirred for 1 h at RT and concentrated. The aqueous solution was acidified to pH=4 with HCl. A white gum formed which was extracted into EtOAc (3×20 mL). The combined organic layers were combined and washed with brine, dried over sodium sulfate, filtered, and concentrated to afford the title compound. (1.4 g, 80% yield). MS (m/z) 408.1 (M+H)⁺

### Step 4: (S)-bis((isobutyryloxy)methyl) 2-(2-ethoxy-4-(4,4,5,5-tetramethyl-1,3,2-dioxaborolan-2-yl)benzamido)succinate

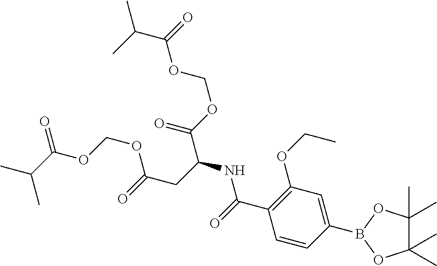 [SMILES: CCOc1cc(B2OC(C)(C)C(C)(C)O2)ccc1C(=O)N[C@@H](CC(=O)OCOC(=O)C(C)C)C(=O)OCOC(=O)C(C)C]

To a solution containing (S)-2-(2-ethoxy-4-(4,4,5,5-tetramethyl-1,3,2-dioxaborolan-2-yl)benzamido)succinic acid (200 mg, 0.491 mmol) and K2CO3 (136 mg, 0.982 mmol) DMF (1 mL) was added chloromethyl isobutyrate (124 μl, 0.982 mmol) and sodium iodide (73.6 mg, 0.491 mmol). The reaction stirred overnight at RT. Additional K2CO3 (136 mg, 0.982 mmol), chloromethyl isobutyrate (124 μl, 0.982 mmol), and sodium iodide (73.6 mg, 0.491 mmol) was added and the reaction was stirred for 48 h. The reaction was poured into water and extracted into EtOAc (3×50 mL). The combined organic layers were washed with brine, dried over sodium sulfate, filtered through a small silica plug, concentrated and dried to afford the title compound as a yellow oil. (295 mg, 99% yield). MS (m/z) 608.4 (M+H)⁺

### Step 5: (S)-bis((isobutyryloxy)methyl) 2-(4-(5-((((R)-2-((R)-1-(N-(benzyloxy)formamido)propyl)heptanamido)methyl)carbamoyl)furan-2-yl)-2-ethoxybenzamido)succinate

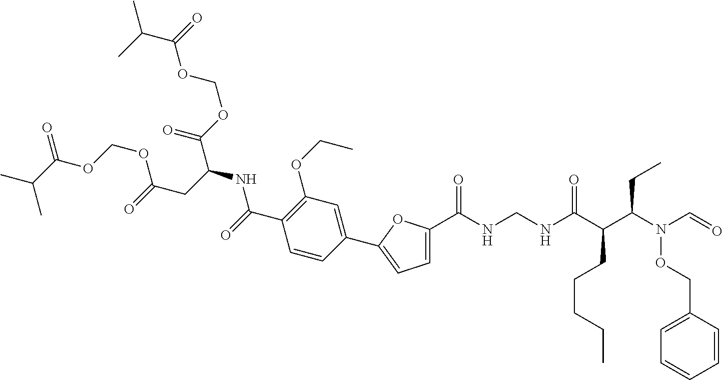 [SMILES: CCCCC[C@@H](C(=O)NCNC(=O)c1ccc(-c2ccc(C(=O)N[C@@H](CC(=O)OCOC(=O)C(C)C)C(=O)OCOC(=O)C(C)C)c(OCC)c2)o1)[C@@H](CC)N(C=O)OCc1ccccc1]

A solution containing (S)-bis((isobutyryloxy)methyl) 2-(2-ethoxy-4-(4,4,5,5-tetramethyl-1,3,2-dioxaborolan-2-yl)benzamido)succinate (128 mg, 0.211 mmol), N—(((R)-2-((R)-1-(N-(benzyloxy)formamido)propyl)heptanamido)methyl)-5-bromofuran-2-carboxamide (100 mg, 0.191 mmol), PdCl2(dppf)-CH2Cl2 (15.63 mg, 0.019 mmol), and K2CO3 (26.5 mg, 0.191 mmol) in 1,4-Dioxane (718 μl):water (239 μl) was stirred for 1 h at RT. (S)-bis((isobutyryloxy)methyl) 2-(2-ethoxy-4-(4,4,5,5-tetramethyl-1,3,2-dioxaborolan-2-yl)benzamido)succinate (128 mg, 0.211 mmol) was added and stirred for 1 h. The reaction was poured into water and extracted into EtOAc (3×50 mL). The combined organic phase was washed with brine, dried over sodium sulfate, filtered, and concentrated in-vacuo. Purification by Si (0-60% EtOAc/Hex) afforded the title compound as a colorless solid. (25 mg, 14% yield).

MS (m/z) 923.8 (M+H)⁺

### Intermediate 134: (S)-dibenzyl 2-(2-ethoxy-4-(5-((((R)-2-((R)-1-(N-((4-(2-oxopyrrolidin-1-yl)benzoyl)oxy)formamido)propyl)heptanamido)methyl)carbamoyl)furan-2-yl)benzamido)succinate

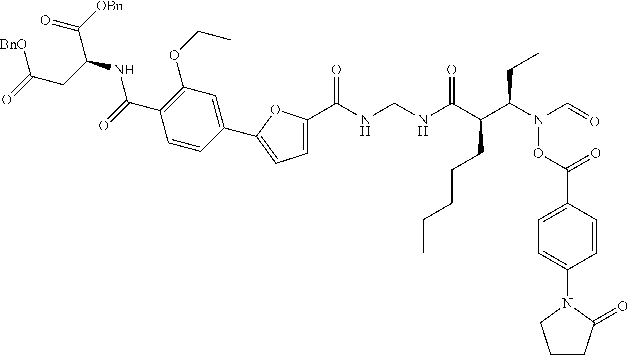 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(N2CCCC2=O)cc1]

A solution containing 4-(2-oxopyrrolidin-1-yl)benzoic acid (0.035 g, 0.171 mmol), HATU (0.071 g, 0.187 mmol), and DIPEA (0.129 mL, 0.738 mmol) in DCM (2.5 mL) was stirred at RT for 20 min. (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.2 g, 0.246 mmol) was added and the reaction was stirred at RT overnight. Water was added and the reaction was extracted with EtOAc. The organic layer was dried over Na₂SO₄ and concentrated. Purification by Si (0-80% EtOAc/Hex) afforded the title compound as a yellow solid. (100 mg, 58% yield). MS (m/z) 500.6 (M/2+H)⁺

### Intermediate 135: (S)-4-benzyl 1-ethyl 2-(2-ethoxy-4-(5-((((R)-2-((R)-1-(N-hydroxyformamido)propyl)heptanamido)methyl)carbamoyl)furan-2-yl)benzamido)succinate

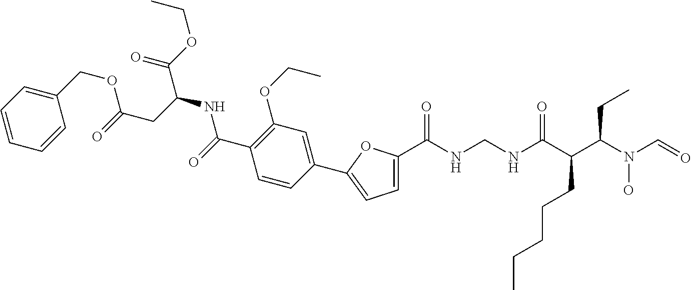 [SMILES: CCCCC[C@@H](C(=O)NCNC(=O)c1ccc(-c2ccc(C(=O)N[C@@H](CC(=O)OCc3ccccc3)C(=O)OCC)c(OCC)c2)o1)[C@@H](CC)N(O)C=O]

### Step 1: (S)-4-(benzyloxy)-2-(4-(5-(tert-butoxycarbonyl)furan-2-yl)-2-ethoxybenzamido)-4-oxobutanoic Acid

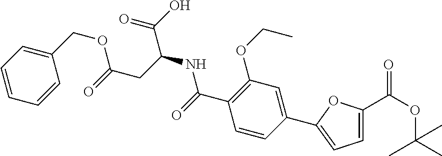 [SMILES: CCOc1cc(-c2ccc(C(=O)OC(C)(C)C)o2)ccc1C(=O)N[C@@H](CC(=O)OCc1ccccc1)C(=O)O]

N₂ was bubbled through a solution containing (S)-4-(benzyloxy)-2-(2-ethoxy-4-(4,4,5,5-tetramethyl-1,3,2-dioxaborolan-2-yl)benzamido)-4-oxobutanoic acid (5.00 g, 10.05 mmol) in 1,4-Dioxane (67.0 mL) for 30 min. Tert-butyl 5-bromofuran-2-carboxylate (2.73 g, 11.06 mmol) and PdCl2 (dppf) (0.452 g, 0.553 mmol) were added followed by addition of sodium carbonate (1 M) (30.2 mL, 30.2 mmol). The resulting mixture was heated to 50° C. under N₂ for 15 min. Excess water was added and the mixture was cooled to RT. EtOAc (200 mL) and water (200 mL) were added and the phases were separated. The organics were washed with brine and the combined aqueous phases were extracted with EtOAc (200 mL). The combined organics were dried over MgSO₄, filtered, and concentrated. Purification by Si (0-100% EtOAc/Hex) afforded the title compound as a orange oil. (1.8 g, 33% yield). MS (m/z) 538.2 (M+H)⁺

### Step 2: (S)-4-benzyl 1-ethyl 2-(4-(5-(tert-butoxycarbonyl)furan-2-yl)-2-ethoxybenzamido)succinate

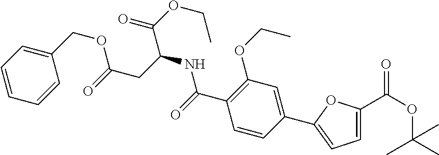 [SMILES: CCOC(=O)[C@H](CC(=O)OCc1ccccc1)NC(=O)c1ccc(-c2ccc(C(=O)OC(C)(C)C)o2)cc1OCC]

ethyl iodide (1.076 mL, 13.32 mmol) was added to a stirring slurry containing (S)-4-(benzyloxy)-2-(4-(5-(tert-butoxycarbonyl)furan-2-yl)-2-ethoxybenzamido)-4-oxobutanoic acid (1.79 g, 3.33 mmol) and potassium carbonate (2.301 g, 16.65 mmol) in DMF (16.65 mL). The mixture was stirred at RT for 18 h. Water and EtOAc were added and the organic phase was washed with brine (3×). The combined washes were extracted with EtOAc, and the extracts were combined with the washed EtOAc layer. The combined organic phase was dried over MgSO4, filtered, and concentrated. Purification by Si (0-100% EtOAc/Hex) afforded the title compound as a colorless solid. (1.5 g, 76% yield). MS (m/z) 566.3 (M+H)⁺

### Step 3: (S)-5-(4-((4-(benzyloxy)-1-ethoxy-1,4-dioxobutan-2-yl)carbamoyl)-3-ethoxyphenyl)furan-2-carboxylic Acid

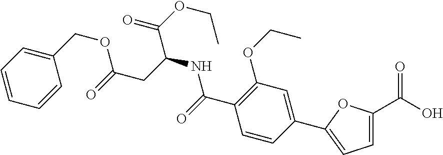 [SMILES: CCOC(=O)[C@H](CC(=O)OCc1ccccc1)NC(=O)c1ccc(-c2ccc(C(=O)O)o2)cc1OCC]

TFA (5.97 mL, 77 mmol) was added slowly to a stirring 0° C. solution containing (S)-4-benzyl 1-ethyl 2-(4-(5-(tert-butoxycarbonyl)furan-2-yl)-2-ethoxybenzamido)succinate (1.46 g, 2.58 mmol) in DCM (7.82 mL). The resulting mixture was stirred for 30 min at 0° C., and was allowed to come to RT. After 4 h, the mixture was then concentrated (azeotroping several times with PhMe) to afford the title compound as a brown solid. (1.4 g, ˜100% yield). MS (m/z) 510.1 (M+H)⁺

### Step 4: (S)-4-benzyl 1-ethyl 2-(2-ethoxy-4-(5-((((R)-2-((R)-1-(N-hydroxyformamido)propyl)heptanamido)methyl)carbamoyl)furan-2-yl)benzamido)succinate

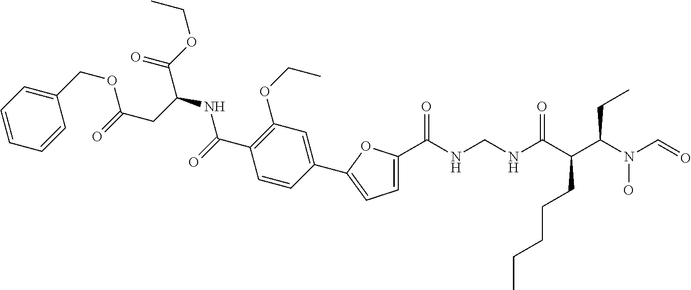 [SMILES: CCCCC[C@@H](C(=O)NCNC(=O)c1ccc(-c2ccc(C(=O)N[C@@H](CC(=O)OCc3ccccc3)C(=O)OCC)c(OCC)c2)o1)[C@@H](CC)N(O)C=O]

To a solution containing (R)—N-(aminomethyl)-2-((R)-1-(N-hydroxyformamido)propyl)heptanamide (0.784 g, 3.02 mmol) in DMF (13.7 mL) was added CDI (0.512 g, 3.16 mmol). The mixture was stirred for 1 h. (S)-5-(4-((4-(benzyloxy)-1-ethoxy-1,4-dioxobutan-2-yl)carbamoyl)-3-ethoxyphenyl)furan-2-carboxylic acid (1.4 g, 2.75 mmol) was added. The mixture was stirred for 18 h. EtOAc was added, and the organic phase was washed with saturated NaHCO₃ followed by brine. The combined washes were extracted with EtOAc (1×). The EtOAc layers were combined and dried over MgSO₄, filtered, and concentrated. The crude material was taken up in MeOH and water and heated to reflux for 18 h and concentrated. Purification by Si (0-10% MeOH/DCM) afforded the title compound as a brown foam. (1.04 g, 51% yield). MS (m/z) 751.2 (M+H)⁺

### Intermediate 136: (S)-4-benzyl 1-tert-butyl 2-(4-(5-((((R)-2-((R)-1-(N-(benzyloxy)formamido)propyl)heptanamido)methyl)carbamoyl)furan-2-yl)-2-ethoxybenzamido)succinate

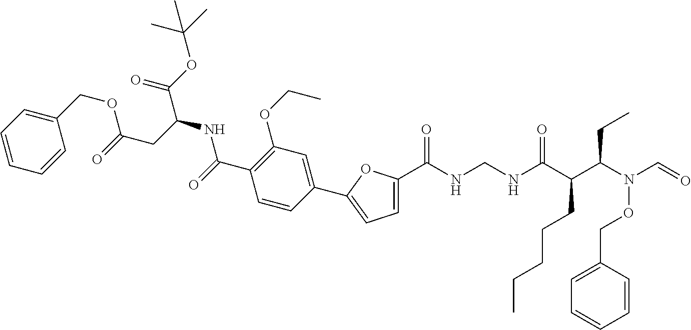 [SMILES: CCCCC[C@@H](C(=O)NCNC(=O)c1ccc(-c2ccc(C(=O)N[C@@H](CC(=O)OCc3ccccc3)C(=O)OC(C)(C)C)c(OCC)c2)o1)[C@@H](CC)N(C=O)OCc1ccccc1]

### Step 1: (S)-4-benzyl 1-tert-butyl 2-(4-bromo-2-ethoxybenzamido)succinate

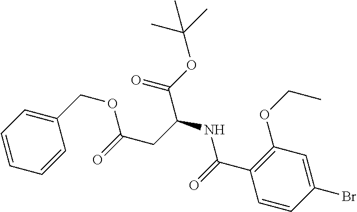 [SMILES: CCOc1cc(Br)ccc1C(=O)N[C@@H](CC(=O)OCc1ccccc1)C(=O)OC(C)(C)C]

DIPEA (7.48 mL, 42.8 mmol) was added to a stirring solution containing 4-bromo-2-ethoxybenzoic acid (3.5 g, 14.28 mmol), (S)-4-(benzyloxy)-1-(tert-butoxy)-1,4-dioxobutan-2-aminium chloride (4.96 g, 15.71 mmol), and HATU (6.52 g, 17.14 mmol) in DMF (40 mL). The resulting solution was stirred at RT for 1 h. Water was added followed by EtOAc. The organics were washed with brine (2×). The combined washes were back-extracted using EtOAc. The combined organics were dried over MgSO4, filtered, and concentrated. Purification by Si (0-100% EtOAc/Hex) afforded the title compound as a colorless oil. (7.03 g, 100% yield). MS (m/z) 506.0 (M+H)⁺

### Step 2: (S)-4-benzyl 1-tert-butyl 2-(2-ethoxy-4-(4,4,5,5-tetramethyl-1,3,2-dioxaborolan-2-yl)benzamido)succinate

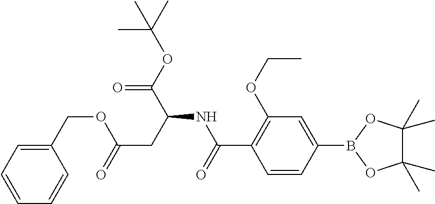 [SMILES: CCOc1cc(B2OC(C)(C)C(C)(C)O2)ccc1C(=O)N[C@@H](CC(=O)OCc1ccccc1)C(=O)OC(C)(C)C]

To a N₂ degassed solution containing (S)-4-benzyl 1-tert-butyl 2-(4-bromo-2-ethoxybenzamido)succinate (7.03 g, 13.88 mmol) in 1,4-Dioxane (69.4 mL) was added bis(pinacolato)diboron (4.23 g, 16.66 mmol), potassium acetate (4.09 g, 41.6 mmol), and PdCl2(dppf)-CH2Cl2 (1.134 g, 1.388 mmol). The resulting mixture was heated to 90° C. for 12 h. Water was added to the cooled reaction, and the crude mixture was filtered through a pad of Celite. The Celite was washed thoroughly with excess EtOAc (1 L). The layers were separated and additional organics were extracted from the aqueous layer with EtOAc (2×1 L). The combined organics were washed with brine (3×), dried over MgSO₄, filtered, and concentrated. Purification by Si (0-100% EtOAc/Hex) afforded the title compound as a colorless oil. (1.14 g, 6% yield). MS (m/z) 554.4 (M+H)⁺

### Step 3: (S)-4-benzyl 1-tert-butyl 2-(4-(5-((((R)-2-((R)-1-(N-(benzyloxy)formamido)propyl)heptanamido)methyl)carbamoyl)furan-2-yl)-2-ethoxybenzamido)succinate

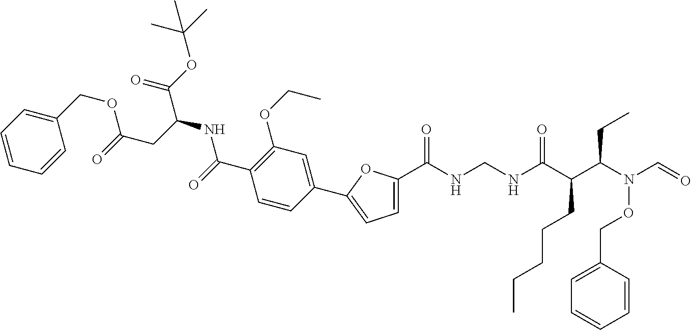 [SMILES: CCCCC[C@@H](C(=O)NCNC(=O)c1ccc(-c2ccc(C(=O)N[C@@H](CC(=O)OCc3ccccc3)C(=O)OC(C)(C)C)c(OCC)c2)o1)[C@@H](CC)N(C=O)OCc1ccccc1]

To a solution containing (S)-4-benzyl 1-tert-butyl 2-(2-ethoxy-4-(4,4,5,5-tetramethyl-1,3,2-dioxaborolan-2-yl)benzamido)succinate (1.14 g, 0.824 mmol), N—(((R)-2-((R)-1-(N-(benzyloxy)formamido)propyl)heptanamido)methyl)-5-bromofuran-2-carboxamide (0.473 g, 0.906 mmol), and PdCl2(dppf)-CH2Cl2 (0.037 g, 0.045 mmol) in 1,4-Dioxane (4.12 mL) was added sodium carbonate (2.472 mL, 2.472 mmol). The resulting mixture was stirred at RT for 15 min. Water was added followed by EtOAc. The organics were washed with brine (2×). The combined washes were back-extracted using EtOAc. The combined organics were dried over MgSO4, filtered, and concentrated. Purification by Si (0-100% EtOAc/Hex) afforded the title compound as a tan solid. (379 mg, 41% yield). MS (m/z) 869.6 (M+H)⁺

### Intermediate 137: (((3-(5-((((R)-2-((R)-1-(N-(benzyloxy)formamido)propyl)heptanamido)methyl)carbamoyl)furan-2-yl)-5-ethoxyphenyl)phosphoryl)bis(oxy))bis(methylene) Diacetate

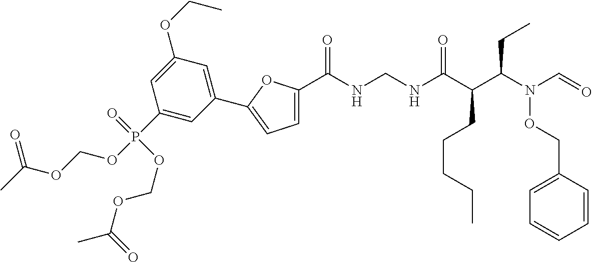 [SMILES: CCCCC[C@@H](C(=O)NCNC(=O)c1ccc(-c2cc(OCC)cc(P(=O)(OCOC(C)=O)OCOC(C)=O)c2)o1)[C@@H](CC)N(C=O)OCc1ccccc1]

To a solution containing (3-(5-((((R)-2-((R)-1-(N-(benzyloxy)formamido)propyl)heptanamido)methyl)carbamoyl)furan-2-yl)-5-ethoxyphenyl)phosphonic acid (0.135 g, 0.210 mmol) in MeCN (1 mL) was added bromoethyl acetate (0.048 g, 0.315 mmol) and DIPEA (0.055 mL, 0.315 mmol). The reaction was stirred at 60° C. for 18 h. Water was added and the reaction was extracted with EtOAc, dried over Na₂SO₄ and concentrated. Purification by Si (0-100% EtOAc/Hex) afforded the title compound as a tan solid. (63 mg, 38% yield). MS (m/z) 788.3 (M+H)⁺

### Intermediate 138: (S)-1-((benzoyloxy)methyl) 4-benzyl 2-(4-(5-((((R)-2-((R)-1-(N-(benzyloxy)formamido)propyl)heptanamido)methyl)carbamoyl)furan-2-yl)-2-ethoxybenzamido)succinate

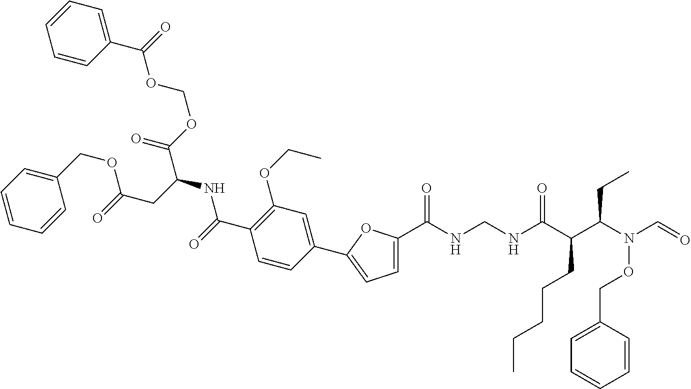 [SMILES: CCCCC[C@@H](C(=O)NCNC(=O)c1ccc(-c2ccc(C(=O)N[C@@H](CC(=O)OCc3ccccc3)C(=O)OCOC(=O)c3ccccc3)c(OCC)c2)o1)[C@@H](CC)N(C=O)OCc1ccccc1]

To a solution containing (S)-4-(benzyloxy)-2-(4-(5-((((R)-2-((R)-1-(N-(benzyloxy)formamido)propyl)heptanamido)methyl)carbamoyl)furan-2-yl)-2-ethoxybenzamido)-4-oxobutanoic acid (200 mg, 0.246 mmol) and sodium carbonate (104 mg, 0.984 mmol) in DMF (2.5 mL) was added chloromethyl benzoate (136 μl, 0.984 mmol) and allowed to stir at RT for 18 h. The reaction was poured into water and extracted into EtOAc (3×50 mL). The combined organic layers were washed with water and brine then dried over sodium sulfate and filtered through a plug of silica and concentrated to afford the title compound as a brown oil. (264 mg, 74% yield). MS (m/z) 947.8 (M+H)⁺

### Intermediate 139: (S)-4-tert-butyl 1-((5-methyl-2-oxo-1,3-dioxol-4-yl)methyl) 2-(2-ethoxy-4-(5-((((R)-2-((R)-1-(N-((4-methoxybenzyl)oxy)formamido)propyl)heptanamido)methyl)carbamoyl)furan-2-yl)benzamido)succinate

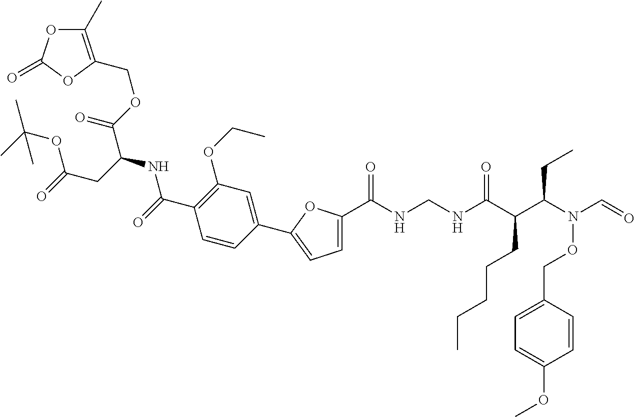 [SMILES: CCCCC[C@@H](C(=O)NCNC(=O)c1ccc(-c2ccc(C(=O)N[C@@H](CC(=O)OC(C)(C)C)C(=O)OCc3oc(=O)oc3C)c(OCC)c2)o1)[C@@H](CC)N(C=O)OCc1ccc(OC)cc1]

### Step 1: (S)-4-tert-butyl 1-methyl 2-(4-bromo-2-ethoxybenzamido)succinate

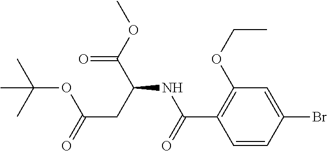 [SMILES: CCOc1cc(Br)ccc1C(=O)N[C@@H](CC(=O)OC(C)(C)C)C(=O)OC]

A solution containing 4-bromo-2-ethoxybenzoic acid (1.07 g, 4.37 mmol), HATU (1.826 g, 4.80 mmol) and DIPEA (0.839 mL, 4.80 mmol) stirred in MeCN (22 mL) was stirred for 30 min. (S)-4-tert-butyl 1-methyl 2-aminosuccinate, Hydrochloride (1.047 g, 4.37 mmol) in DMF (50 mL) was added. The reaction was stirred for 2 h at RT and poured into water and extracted into EtOAc (3×100 mL). The combined organic layers were washed with brine, dried over sodium sulfate, filter through a plug of celite and concentrated in-vacuo to a colorless oil. Purification by Si (10-80% EtOAc/Hex) afforded the title compound as a colorless oil. (683 mg, 36% yield). MS (m/z) 430.1 (M+H)⁺

### Step 2: (S)-4-tert-butyl 1-methyl 2-(2-ethoxy-4-(4,4,5,5-tetramethyl-1,3,2-dioxaborolan-2-yl)benzamido)succinate

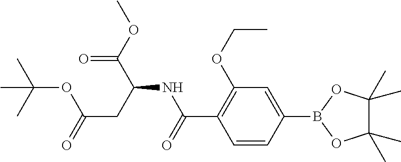 [SMILES: CCOc1cc(B2OC(C)(C)C(C)(C)O2)ccc1C(=O)N[C@@H](CC(=O)OC(C)(C)C)C(=O)OC]

A solution containing (S)-4-tert-butyl 1-methyl 2-(4-bromo-2-ethoxybenzamido)succinate (686 mg, 1.594 mmol), bis(pinacolato)diboron (607 mg, 2.391 mmol), PdCl2(dppf)-CH2Cl2 (52.1 mg, 0.064 mmol), and potassium acetate (469 mg, 4.78 mmol) in 1,4-dioxane (8 mL) was stirred at 100° C. for 18 h. Bis(pinacolato)diboron (607 mg, 2.391 mmol) and PdCl2(dppf)-CH2Cl2 (52.1 mg, 0.064 mmol) was added and stirred for 1 h at 110° C. The reaction was cooled and poured into water and extracted into EtOAc (3×50 mL). The combined organic layers were washed with brine, dried over sodium sulfate and decolorizing C and filtered through a short silica plug. The filtrate was concentrated and dried overnight to afford the title compound as a yellow oil. (827 mg, 98% yield). MS (m/z) 478.3 (M+H)⁺

### Step 3: (S)-4-tert-butyl 1-methyl 2-(2-ethoxy-4-(5-((((R)-2-((R)-1-(N-((4-methoxybenzyl)oxy)formamido)propyl)heptanamido)methyl)carbamoyl)furan-2-yl)benzamido)succinate

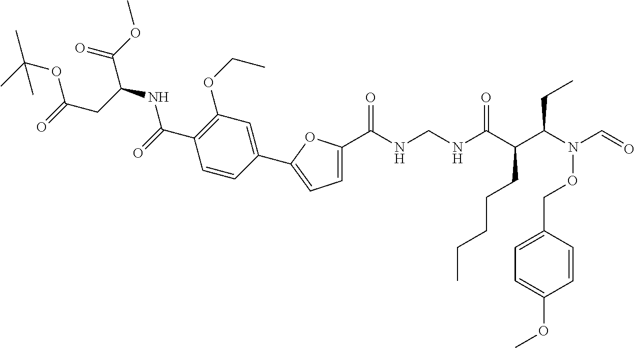 [SMILES: CCCCC[C@@H](C(=O)NCNC(=O)c1ccc(-c2ccc(C(=O)N[C@@H](CC(=O)OC(C)(C)C)C(=O)OC)c(OCC)c2)o1)[C@@H](CC)N(C=O)OCc1ccc(OC)cc1]

To a solution containing (S)-4-tert-butyl 1-methyl 2-(2-ethoxy-4-(4,4,5,5-tetramethyl-1,3,2-dioxaborolan-2-yl)benzamido)succinate (587 mg, 1.231 mmol) in 1,4-Dioxane (3 mL):2M Na2CO3 (3 mL) was added 5-bromo-N—(((R)-2-((R)-1-(N-((4-methoxybenzyl)oxy)formamido)propyl)heptanamido)methyl)furan-2-carboxamide (618 mg, 1.119 mmol), and PdCl2(dppf)-CH2Cl2 (91 mg, 0.112 mmol). The reaction was stirred at 50° C. for 1 h and 1 h at 70° C. The reaction was cooled to RT. Poured into water and extracted into EtOAc (3×). The combined organic layers were dried over sodium sulfate and filtered through a plug of silica and concentrated. Purification by Si (0-80% EtOAc/Hex) afforded the title compound as a colorless oil. (250 mg, 27% yield). MS (m/z) 823.6 (M+H)⁺

### Step 4: (S)-4-(tert-butoxy)-2-(2-ethoxy-4-(5-((((R)-2-((R)-1-(N-((4-methoxybenzyl)oxy)formamido)propyl)heptanamido)methyl)carbamoyl)furan-2-yl)benzamido)-4-oxobutanoic Acid

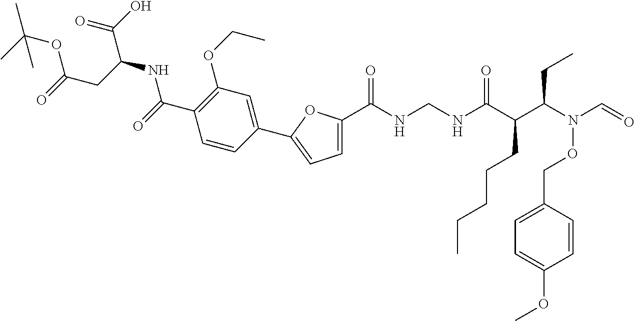 [SMILES: CCCCC[C@@H](C(=O)NCNC(=O)c1ccc(-c2ccc(C(=O)N[C@@H](CC(=O)OC(C)(C)C)C(=O)O)c(OCC)c2)o1)[C@@H](CC)N(C=O)OCc1ccc(OC)cc1]

To a solution containing (S)-4-tert-butyl 1-methyl 2-(2-ethoxy-4-(5-((((R)-2-((R)-1-(N-((4-methoxybenzyl)oxy)formamido)propyl)heptanamido)methyl)carbamoyl)furan-2-yl)benzamido)succinate (250 mg, 0.304 mmol) in THF (2278 μl):water (759 μl) was added LiOH (22 mg, 0.911 mmol). The reaction was stirred for 18 h at RT. The reaction was diluted with water and acidified with 1N HCl and then extracted into EtOAc (3×25 mL). The combined organic layers were washed with brine, dried over sodium sulfate, filtered, and concentrated in-vacuo to afford the title compound as a yellow oil. (200 mg, 81% yield). MS (m/z) 809.7 (M+H)⁺

### Step 4: (S)-4-tert-butyl 1-((5-methyl-2-oxo-1,3-dioxol-4-yl)methyl) 2-(2-ethoxy-4-(5-((((R)-2-((R)-1-(N-((4-methoxybenzyl)oxy)formamido)propyl)heptanamido)methyl)carbamoyl)furan-2-yl)benzamido)succinate

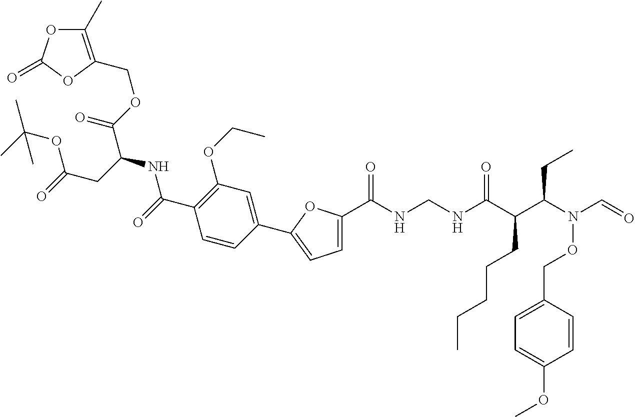 [SMILES: CCCCC[C@@H](C(=O)NCNC(=O)c1ccc(-c2ccc(C(=O)N[C@@H](CC(=O)OC(C)(C)C)C(=O)OCc3oc(=O)oc3C)c(OCC)c2)o1)[C@@H](CC)N(C=O)OCc1ccc(OC)cc1]

To a solution containing (S)-4-(tert-butoxy)-2-(2-ethoxy-4-(5-((((R)-2-((R)-1-(N-((4-methoxybenzyl)oxy)formamido)propyl)heptanamido)methyl)carbamoyl)furan-2-yl)benzamido)-4-oxobutanoic acid (200 mg, 0.247 mmol) in DMF (1236 μl) was added 4-(chloromethyl)-5-methyl-1,3-dioxol-2-one (108 μl, 0.989 mmol) and sodium carbonate (105 mg, 0.989 mmol). The reaction was stirred for 18 h. The reaction was poured into water and extracted into EtOAc (3×50 mL). The combined organic layers were washed with brine, dried over sodium sulfate, filtered, and concentrated. Purification by Si (0-100% EtOAc/Hex) afforded the title compound as a colorless solid. (50 mg, 22% yield). MS (m/z) 922.8 (M+H)⁺

### Intermediate 140: 5-bromo-N—(((R)-2-((R)-1-(N-((4-methoxybenzyl)oxy)formamido)propyl)heptanamido)methyl)furan-2-carboxamide

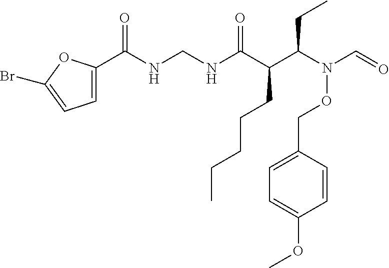 [SMILES: CCCCC[C@@H](C(=O)NCNC(=O)c1ccc(Br)o1)[C@@H](CC)N(C=O)OCc1ccc(OC)cc1]

### Step 1: 5-bromo-N—(((R)-2-((R)-1-(N-hydroxyformamido)propyl)heptanamido)methyl)furan-2-carboxamide

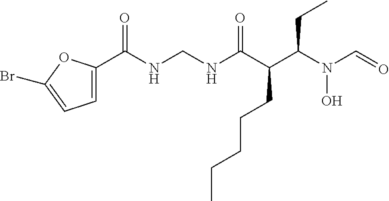 [SMILES: CCCCC[C@@H](C(=O)NCNC(=O)c1ccc(Br)o1)[C@@H](CC)N(O)C=O]

A solution containing 5-bromofuran-2-carboxylic acid (3 g, 15.71 mmol), HATU (6.57 g, 17.28 mmol), and DIPEA (3.02 mL, 17.28 mmol) in MeCN (79 mL) was stirred for 30 min. (R)—N-(aminomethyl)-2-((R)-1-(N-hydroxyformamido)propyl)heptanamide (4.07 g, 15.71 mmol) in DMF (100 mL) was added and the reaction stirred for 2 h at RT. The reaction was poured into water and extracted into EtOAc (3×100 mL). The combined organic layers were washed with brine, dried over sodium sulfate, filter through a plug of celite and concentrated in-vacuo to an pale orange solid. The solids were suspended in diethyl ether, stirred for 30 min, filtered, and dried to afford the title compound as a colorless solid. (4.9 g, 72% yield). MS (m/z) 432.1 (M+H)⁺

### Step 2: 5-bromo-N—(((R)-2-((R)-1-(N-((4-methoxybenzyl)oxy)formamido)propyl)heptanamido)methyl)furan-2-carboxamide

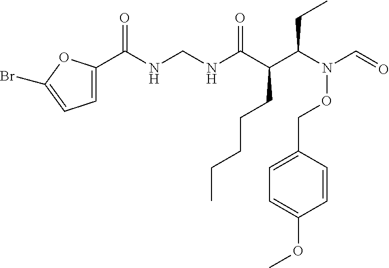 [SMILES: CCCCC[C@@H](C(=O)NCNC(=O)c1ccc(Br)o1)[C@@H](CC)N(C=O)OCc1ccc(OC)cc1]

A solution containing 5-bromo-N—(((R)-2-((R)-1-(N-hydroxyformamido)propyl)heptanamido)methyl)furan-2-carboxamide (1 g, 2.313 mmol), 1-(chloromethyl)-4-methoxybenzene (345 μL, 3.47 mmol) and DIPEA (1010 μL, 5.78 mmol) in DCM (11.6 mL) was stirred for 18 h at RT. DMF (20 mL) was added and heated to 90° C. for 1 h. NaI (catalytic) was added followed by 1-(chloromethyl)-4-methoxybenzene (345 μL, 3.47 mmol). The reaction was stirred for 18 h and heated to 110° C. for 2 h. The reaction was cooled to RT and poured into water and extracted into EtOAc (3×50 mL). The combined organic layers were washed with brine, dried over sodium sulfate, and filtered through a plug of silica. The filtrate was concentrated in-vacuo to afford the title compound as an orange oil. (618 mg, 48% yield). MS (m/z) 552.2 (M+H)⁺

### Intermediate 141: (3-ethoxy-5-(5-((((R)-2-((R)-1-(N-hydroxyformamido)propyl)heptanamido)methyl)carbamoyl)furan-2-yl)phenyl)phosphonic Acid

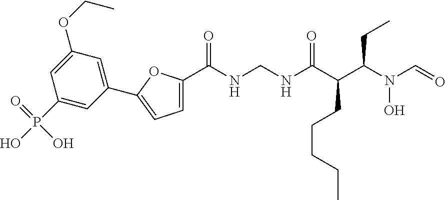 [SMILES: CCCCC[C@@H](C(=O)NCNC(=O)c1ccc(-c2cc(OCC)cc(P(=O)(O)O)c2)o1)[C@@H](CC)N(O)C=O]

### Step 1: Dimethyl (3-ethoxy-5-(5-((((R)-2-((R)-1-(N-hydroxyformamido)propyl)heptanamido)methyl)carbamoyl)furan-2-yl)phenyl)phosphonate

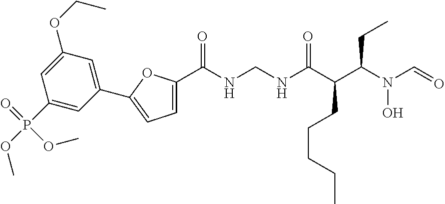 [SMILES: CCCCC[C@@H](C(=O)NCNC(=O)c1ccc(-c2cc(OCC)cc(P(=O)(OC)OC)c2)o1)[C@@H](CC)N(O)C=O]

A solution containing 5-(3-(dimethoxyphosphoryl)-5-ethoxyphenyl)furan-2-carboxylic acid (21.24 g, 62.4 mmol) in MeCN (200 mL) was treated with TEA (12.18 mL, 87 mmol) followed by HATU (24.92 g, 65.5 mmol) and the reaction was stirred for 20 min. in a separate 2 L flask, (R)—N-(aminomethyl)-2-((R)-1-(N-hydroxyformamido)propyl)heptanamide (17.32 g, 66.8 mmol) was suspended in MeCN (200 mL) and TEA (24 mL, 174 mmol) was added followed by TMSCl (16.75 mL, 131 mmol). The reaction was then immersed in an ice bath for 20 mins. The HATU adduct solution above was added slowly to the amine silane mixture (˜25 mins). The ice bath was removed and the reaction was stirred for 1.5 h. TBAF (1M in THF) (250 mL, 250 mmol) was added and the reaction stirred for 20 min and then concentrated. To the residue was added water (700 mL) and stirred for 20 min. The solid was collected by filtration and washed with water. The water layer was extracted with DCM (300 mL). The DCM layer was used to dissolve the solid collected above and passed through a hydrophobic cartridge and concentrated. Purification by Si (0-100% EtOAc/Hex followed by 0-10% MeOH/DCM) afforded the title compound mixed with impurities. The material was taken up in DCM (350 mL), washed with water, 0.5 N HCl, and then with water (2×) again. The organic layer was passed through a hydrophobic cartridge and concentrated. EtOAc (160 mL) was added to the foam residue and the flask was spun on the rotovap with temperature set at 55° C. until the foam completely dissolved. After cooled down to RT, hexane was added slowly with stirring. Some solid formed upon contact but dissolved after stirring for a few seconds. Stopped adding hexane (˜160 mL) when the solid did not go way. Added a few drops of EtOAc just enough to dissolve the solid and the reaction was stirred for 48 h. The solid precipitated was collected by filtration and air dried to afford the title compound as colorless solid. (22.5 g, 62% yield). MS (m/z) 582.3 (M+H)⁺

### Step 2: (3-ethoxy-5-(5-((((R)-2-((R)-1-(N-hydroxyformamido)propyl)heptanamido)methyl)carbamoyl)furan-2-yl)phenyl)phosphonic Acid

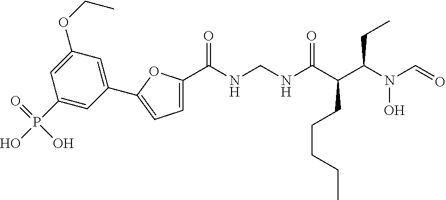 [SMILES: CCCCC[C@@H](C(=O)NCNC(=O)c1ccc(-c2cc(OCC)cc(P(=O)(O)O)c2)o1)[C@@H](CC)N(O)C=O]

TMSBr (17.96 mL, 138 mmol) was added slowly to a solution containing dimethyl (3-ethoxy-5-(5-((((R)-2-((R)-1-(N-hydroxyformamido)propyl)heptanamido)methyl)carbamoyl)furan-2-yl)phenyl)phosphonate (26.83 g, 46.1 mmol) in DCM (370 mL) and MeCN (100 mL) at 0° C. The reaction was stirred at 0° C. for 5 mins. Ice bath was removed and the reaction was stirred for 3.5 h. TMSBr (5 mL) was added again and the reaction was stirred for 1.5 h. HCl (1M, 200 mL) was then added fast drop wise with vigorous stirring. Stirring was stopped and immersed the flask in an ice bath for 10 mins. The liquid was decanted and the resulting residue was washed with water (3×), MeCN (3×). At this time the residue became a hard solid. The solid was broken up and washed with EtOAc (2×) and taken up in warm MeOH. The solution was filtered while warm. The filtrate was cooled to RT with stirring and MeCN (440 mL) was added with stirring. The solution was stirred for 4 h at which time the resulting solids were collected by filtration, washed with ACN and air dried overnight to afford the title compound as a colorless solid. (15.6 g, 61% yield). MS (m/z) 554.3 (M+H)⁺

### Intermediate 142: 4-(iodomethyl)-5-methyl-1,3-dioxol-2-one

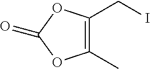

A suspension containing 4-(chloromethyl)-5-methyl-1,3-dioxol-2-one (0.5 g, 3.37 mmol) and sodium iodide (0.908 g, 6.06 mmol) in MeCN (1.122 mL) was stirred at 30° C. for 30 min. MeOH (˜1 mL) was added and the reaction was filtered and washed with DCM. Water was added and layers were separated. The organic layer was washed with a solution of Na2S2O3 (2%) and passed through a hydrophobic cartridge and concentrated to afford the title compound as a brown oil. (0.7 g, 87% yield). MS (m/z) 241.0 (M+H)⁺

### Intermediate 143: (S)-5-(4-((1,4-diethoxy-1,4-dioxobutan-2-yl)carbamoyl)-3-ethoxyphenyl)furan-2-carboxylic Acid

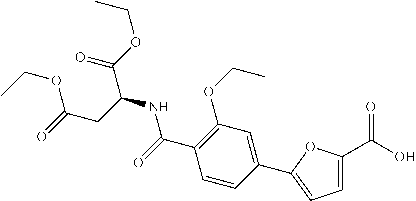 [SMILES: CCOC(=O)C[C@H](NC(=O)c1ccc(-c2ccc(C(=O)O)o2)cc1OCC)C(=O)OCC]

### Step 1: (S)-diethyl 2-(4-bromo-2-ethoxybenzamido)succinate

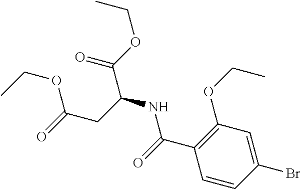 [SMILES: CCOC(=O)C[C@H](NC(=O)c1ccc(Br)cc1OCC)C(=O)OCC]

To a solution containing (S)-diethyl 2-aminosuccinate, Hydrochloride (5.53 g, 24.48 mmol) and 4-bromo-2-ethoxybenzoic acid (5 g, 20.40 mmol) in DCM (40 mL) at RT was added HATU (9.31 g, 24.48 mmol) followed by DIPEA (10.69 mL, 61.2 mmol). The reaction mixture was stirred at RT for 2 h. LCMS showed desired product-reaction mixture was diluted with NH4Cl aq. and washed with DCM. Organic layer was dried over MgSO4, filtered and concentrated onto Celite. Purification by Si (0-100% EtOAc/Hex) afforded the title compound as a pale yellow oil. (8.1 g, 95% yield). MS (m/z) 416.0 (M+H)⁺

### Step 2: (S)-diethyl 2-(2-ethoxy-4-(4,4,5,5-tetramethyl-1,3,2-dioxaborolan-2-yl)benzamido)succinate

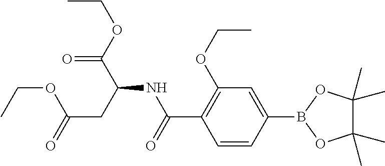 [SMILES: CCOC(=O)C[C@H](NC(=O)c1ccc(B2OC(C)(C)C(C)(C)O2)cc1OCC)C(=O)OCC]

(S)-diethyl 2-(4-bromo-2-ethoxybenzamido)succinate (8.09 g, 19.43 mmol) in 1,4-Dioxane (80 mL) was degassed for 5 min with N2. At this time, 4,4,4′,4′,5,5,5′,5′-octamethyl-2,2′-bi(1,3,2-dioxaborolane) (4.94 g, 19.43 mmol), potassium acetate (5.72 g, 58.3 mmol), and PdCl2(dppf)-CH2Cl2 (1.428 g, 1.749 mmol) were added and degassed for 5 min with N2 and then heated to 90° C. for 6 h under N2. The reaction mixture was cooled, diluted with EtOAc and brine and filtered through celite and washed with EtOAc. Organic phase was dried over MgSO4, filtered and concentrated onto SiO2. Purification by Si (0-50% EtOAc/Hex) afforded the title compound. (8.5 g, 94% yield). MS (m/z) 464.3 (M+H)⁺

### Step 3: (S)-diethyl 2-(4-(5-(tert-butoxycarbonyl)furan-2-yl)-2-ethoxybenzamido)succinate

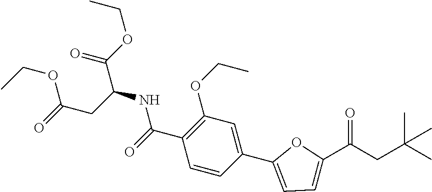 [SMILES: CCOC(=O)C[C@H](NC(=O)c1ccc(-c2ccc(C(=O)CC(C)(C)C)o2)cc1OCC)C(=O)OCC]

(S)-diethyl 2-(2-ethoxy-4-(4,4,5,5-tetramethyl-1,3,2-dioxaborolan-2-yl)benzamido)succinate (8.45 g, 18.24 mmol) in 1,4-Dioxane (50 mL) was degassed for 10 min with N2. At this time, tert-butyl 5-bromofuran-2-carboxylate (4.96 g, 20.06 mmol), sodium carbonate (54.7 mL, 54.7 mmol), and PdCl2(dppf)-CH2Cl2 (0.819 g, 1.003 mmol) were added and degassed for 5 min with N2 and then heated to 50° C. for 20 min under N2. The reaction mixture was cooled and diluted with EtOAc and washed with water. Aqueous phase was back extracted with EtOAc. Organic phase was combined, dried over MgSO4, filtered and concentrated onto SiO2. Purification by Si (0-50% EtOAc/Hex) afforded the title compound. (6.04 g, 66% yield). MS (m/z) 504.3 (M+H)⁺

### Step 4: (S)-5-(4-((1,4-diethoxy-1,4-dioxobutan-2-yl)carbamoyl)-3-ethoxyphenyl)furan-2-carboxylic Acid

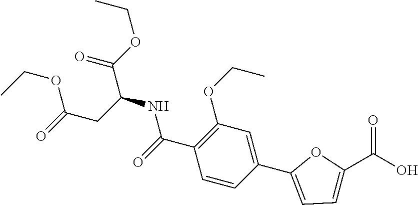 [SMILES: CCOC(=O)C[C@H](NC(=O)c1ccc(-c2ccc(C(=O)O)o2)cc1OCC)C(=O)OCC]

TFA (27.7 mL, 360 mmol) was added slowly to a stirring 0° C. solution of (S)-diethyl 2-(4-(5-(tert-butoxycarbonyl)furan-2-yl)-2-ethoxybenzamido)succinate (6.04 g, 12.00 mmol) in DCM (36.3 mL). The resulting mixture was stirred for 30 min at 0° C., and was allowed to come to RT. After 4 h, the mixture was then concentrated (azeotroping several times with PhMe) to afford the title compound as a brown solid. (5.99 g, 90% yield). MS (m/z) 448.1 (M+H)⁺

### Intermediate 144: (S)-dibenzyl 2-(2-ethoxy-4-(5-((((R)-2-((R)-1-(N-((4-fluoro-2-methylbenzoyl)oxy)formamido)propyl)heptanamido)methyl)carbamoyl)furan-2-yl)benzamido)succinate

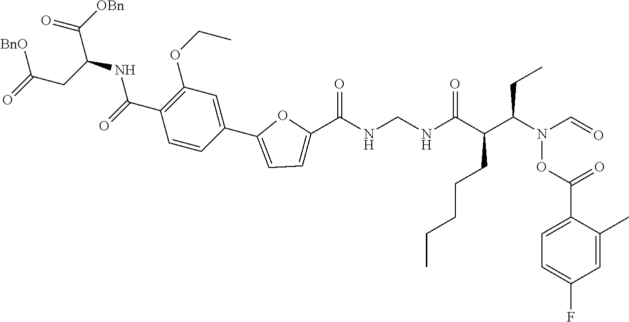 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(F)cc1C]

To a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.1 g, 0.123 mmol) and 4-fluoro-2-methylbenzoyl chloride (0.106 g, 0.615 mmol) in MeCN (2 mL) was added TEA (0.086 mL, 0.615 mmol). The resulting mixture was stirred for 15 min and evaporated. Purification by Si (0-100% EtOAc/Hex) afforded the title compound as a foam. (77 mg, 66% yield). MS (m/z) 949.5 (M+H)⁺

### Intermediate 145: (S)-dibenzyl 2-(2-ethoxy-4-(5-((((R)-2-((R)-1-(N-((piperidine-4-carbonyl)oxy)formamido)propyl)heptanamido)methyl)carbamoyl)furan-2-yl)benzamido)succinate, Trifluoroacetic Acid Salt

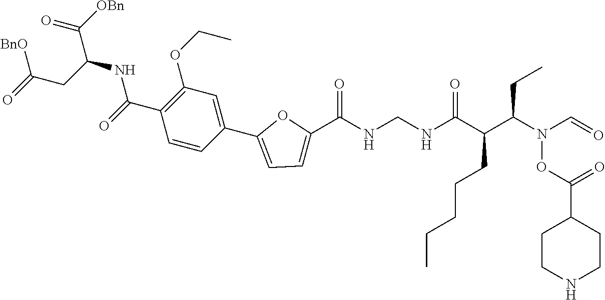 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)C1CCNCC1]

### Step 1: (S)-dibenzyl 2-(4-(5-((((R)-2-((R)-1-(N-((1-(tert-butoxycarbonyl)piperidine-4-carbonyl)oxy)formamido)propyl)heptanamido)methyl)carbamoyl)furan-2-yl)-2-ethoxybenzamido)succinate

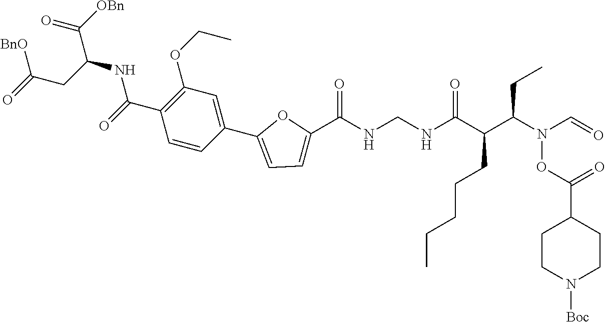 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)C1CCN(C(=O)OC(C)(C)C)CC1]

To a solution containing HATU (0.119 g, 0.314 mmol), N-methylmorpholine (0.069 mL, 0.627 mmol) and 1-(tert-butoxycarbonyl)piperidine-4-carboxylic acid (0.072 g, 0.314 mmol) in MeCN (1.046 mL) was added slowly a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (0.17 g, 0.209 mmol) in MeCN (1.046 mL). The resulting mixture was stirred for 18 h and concentrated. Purification by Si (30-100% EtOAc/Hex) afforded the title compound as a colorless oil. (200 mg, 93% yield). MS (m/z) 1024.8 (M+H)⁺

### Step 2: (S)-dibenzyl 2-(2-ethoxy-4-(5-((((R)-2-((R)-1-(N-((piperidine-4-carbonyl)oxy)formamido)propyl)heptanamido)methyl)carbamoyl)furan-2-yl)benzamido)succinate, Trifluoroacetic Acid Salt

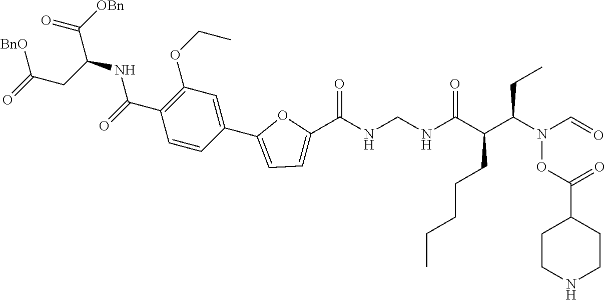 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)C1CCNCC1]

(S)-dibenzyl 2-(4-(5-((((R)-2-((R)-1-(N-((1-(tert-butoxycarbonyl)piperidine-4-carbonyl)oxy)formamido)propyl)heptanamido)methyl)carbamoyl)furan-2-yl)-2-ethoxybenzamido)succinate (0.2 g, 0.195 mmol) was dissolved in DCM (5 mL) and TFA (1.5 mL) was slowly added. The mixture was stirred for 2 h and concentrated to afford the title compound as a clear oil. (200 mg, 92% yield). MS (m/z) 924.7 (M+H)⁺

### Intermediate 146: (S)-dibenzyl 2-(2-ethoxy-4-(5-((((R)-2-((R)-1-(N-((4-(trifluoromethyl)benzoyl)oxy)formamido)propyl)heptanamido)methyl)carbamoyl)furan-2-yl)benzamido)succinate

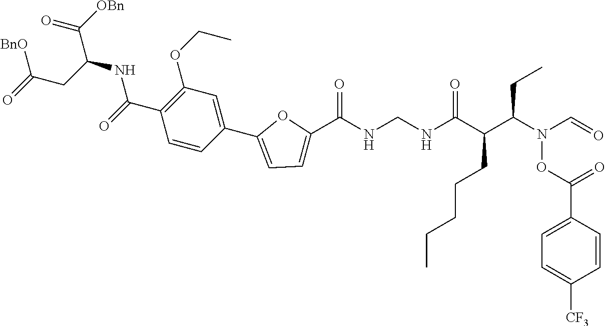 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(C(F)(F)F)cc1]

To a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (200 mg, 0.246 mmol), 2-(3H-[1,2,3]triazolo[4,5-b]pyridin-3-yl)-1,1,3,3-tetramethylisouronium hexafluorophosphate(V) (140 mg, 0.369 mmol), and 4-(trifluoromethyl)benzoic acid (56.1 mg, 0.295 mmol) in MeCN (5 mL) and DMF (5 mL) was added 4-methylmorpholine (0.081 mL, 0.738 mmol). The mixture was stirred at 55° C. for 18 h. The reaction mixture was diluted with EtOAc and washed with water. Purification by Si (0-100% EtOAc/Hex) afforded the title compound as a colorless solid. (202 mg, 79% yield). MS (m/z) 985.9 (M+H)⁺

### Intermediate 147: (S)-dibenzyl 2-(2-ethoxy-4-(5-((((R)-2-((R)-1-(N-((4-nitrobenzoyl)oxy)formamido)propyl)heptanamido)methyl)carbamoyl)furan-2-yl)benzamido)succinate

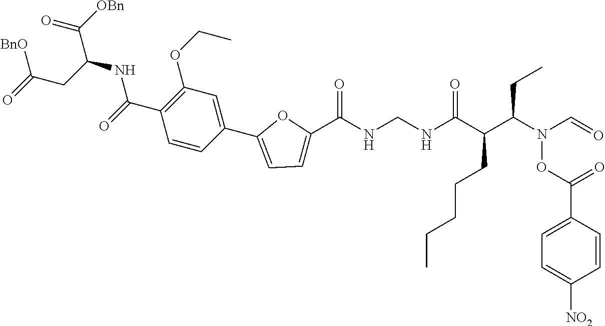 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc([N+](=O)[O-])cc1]

To a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (200 mg, 0.246 mmol), 2-(3H-[1,2,3]triazolo[4,5-b]pyridin-3-yl)-1,1,3,3-tetramethylisouronium hexafluorophosphate(V) (140 mg, 0.369 mmol), and 4-nitrobenzoic acid (49.3 mg, 0.295 mmol) in MeCN (5 mL) and DMF (5 mL) was added 4-methylmorpholine (0.081 mL, 0.738 mmol). The reaction was stirred at 55° C. for 18 h. The reaction mixture was diluted with EtOAc and washed with water. Purification by Si (0-100% EtOAc/Hex followed by 5% MeOH/EtOAc) afforded the title compound as a colorless solid. (192 mg, 79% yield). MS (m/z) 962.7 (M+H)⁺

### Intermediate 148: (S)-dibenzyl 2-(4-(5-((((R)-2-((R)-1-(N-((4-acetoxybenzoyl)oxy)formamido)propyl)heptanamido)methyl)carbamoyl)furan-2-yl)-2-ethoxybenzamido)succinate

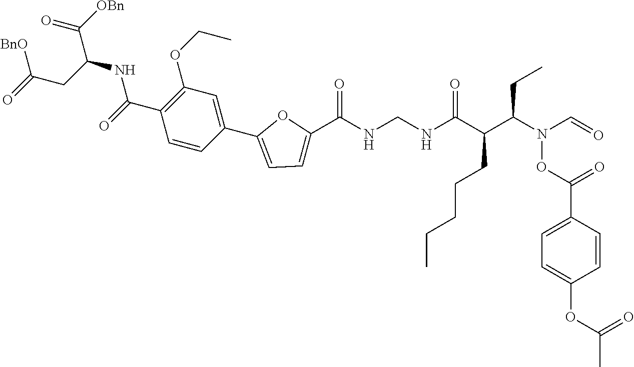 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(OC(C)=O)cc1]

To a solution containing 4-acetoxybenzoic acid (89 mg, 0.492 mmol) in MeCN (1 mL) and DMF (1 mL) was added HATU (281 mg, 0.738 mmol) and N-methylmorpholine (0.135 mL, 1.230 mmol). The reaction was stirred at RT for 15 min. (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (200 mg, 0.246 mmol) was added and stirred at RT for 5 h. Reaction was concentrated diluted with DCM and water (5 mL each) and layers were separated. The organic phase was passed through a phase separator and concentrated. Purification by Si (0-100% EtOAc/Hex) afforded the title compound as a colorless solid. (166 mg, 69% yield). MS (m/z) 976.9 (M+H)⁺

### Intermediate 149: (S)-dibenzyl 2-(2-ethoxy-4-(5-((((R)-2-((R)-1-(N-((2-fluoro-4-methoxybenzoyl)oxy)formamido)propyl)heptanamido)methyl)carbamoyl)furan-2-yl)benzamido)succinate

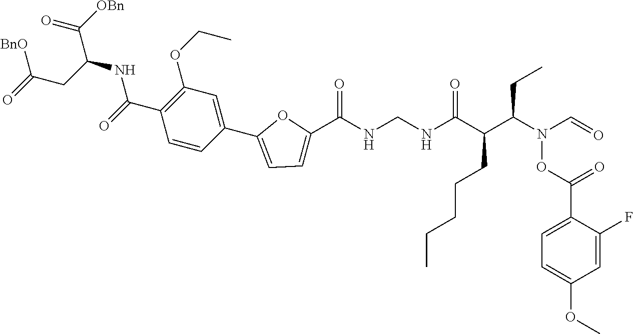 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(OC)cc1F]

To a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (96.1 mg, 0.118 mmol), 2-fluoro-4-methoxybenzoic acid (24.14 mg, 0.142 mmol), and HATU (67.4 mg, 0.177 mmol) in MeCN (0.5 mL) and DMF (0.5 mL) at RT was added N-methylmorpholine (0.039 mL, 0.355 mmol). The reaction was stirred at 50° C. for 18 h. The mixture was partitioned between EtOAc and water, and the organic layer was washed with brine. It was then dried over MgSO₄, filtered, and concentrated. Purification by Si (0-100% EtOAc/Hex) afforded the title compound as a colorless solid. (109 mg, 95% yield). MS (m/z) 965.9 (M+H)⁺

### Intermediate 150: (S)-dibenzyl 2-(2-ethoxy-4-(5-((((R)-2-((R)-1-(N-((4-methoxy-2-(trifluoromethyl)benzoyl)oxy)formamido)propyl)heptanamido)methyl)carbamoyl)furan-2-yl)benzamido)succinate

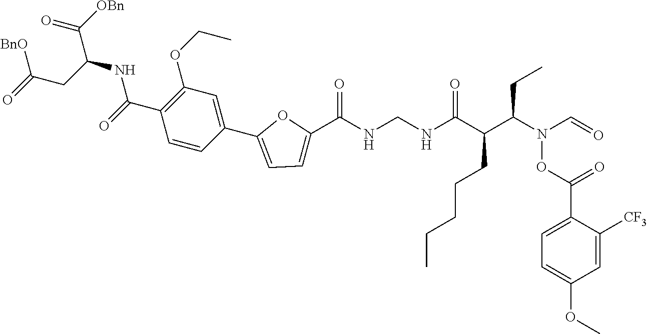 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(OC)cc1C(F)(F)F]

To a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (83.1 mg, 0.102 mmol), 4-methoxy-2-(trifluoromethyl)benzoic acid (27.0 mg, 0.123 mmol), and HATU (58.3 mg, 0.153 mmol) in MeCN (0.5 mL) and DMF (0.5 mL) at RT was added N-methylmorpholine (0.034 mL, 0.307 mmol). The reaction was stirred at 50° C. for 18 h. The mixture was partitioned between EtOAc and water, and the organic layer was washed with brine. It was then dried over MgSO₄, filtered, and concentrated. Purification by Si (0-100% EtOAc/Hex) afforded the title compound as a colorless solid. (81 mg, 78% yield). MS (m/z) 1015.5 (M+H)⁺

### Intermediate 151: (S)-dibenzyl 2-(2-ethoxy-4-(5-((((R)-2-((R)-1-(N-((4-methoxy-2-(trifluoromethoxy)benzoyl)oxy)formamido)propyl)heptanamido)methyl)carbamoyl)furan-2-yl)benzamido)succinate

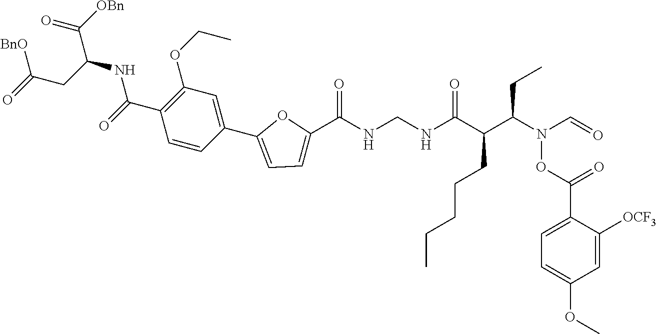 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(OC)cc1OC(F)(F)F]

To a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (76.4 mg, 0.094 mmol), 4-methoxy-2-(trifluoromethoxy)benzoic acid (26.6 mg, 0.113 mmol), and HATU (53.6 mg, 0.141 mmol) in MeCN (0.5 mL) and DMF (0.5 mL) at RT was added N-methylmorpholine (0.031 mL, 0.282 mmol). The reaction was stirred at 50° C. for 18 h. The mixture was partitioned between EtOAc and water, and the organic layer was washed with brine. It was then dried over MgSO₄, filtered, and concentrated. Purification by Si (0-100% EtOAc/Hex) afforded the title compound as a colorless solid. (93 mg, 96% yield). MS (m/z) 1031.6 (M+H)⁺

### Intermediate 152: Dibenzyl ((4-(5-((((R)-2-((R)-1-(N-(benzoyloxy)formamido)propyl)heptanamido)methyl)carbamoyl)furan-2-yl)-2-ethoxybenzamido)methyl)phosphonate

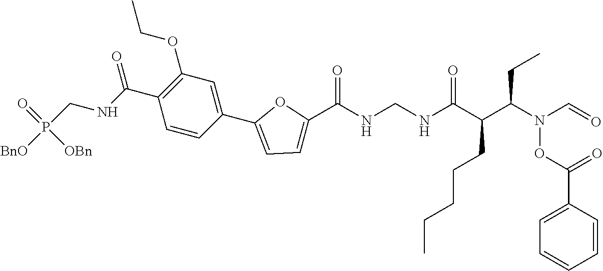 [SMILES: CCCCC[C@@H](C(=O)NCNC(=O)c1ccc(-c2ccc(C(=O)NCP(=O)(OCc3ccccc3)OCc3ccccc3)c(OCC)c2)o1)[C@@H](CC)N(C=O)OC(=O)c1ccccc1]

### Step 1: Dibenzyl ((tritylamino)methyl)phosphonate

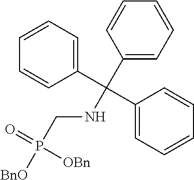 [SMILES: O=P(CNC(c1ccccc1)(c1ccccc1)c1ccccc1)(OCc1ccccc1)OCc1ccccc1]

Paraformaldehyde (1.259 g, 41.9 mmol) and AcOH (0.437 mL, 7.63 mmol) were added to a mixture of triphenylmethanamine (9.99 g, 38.5 mmol) in Toluene (201 mL). The reaction was heated to 80° C. for 1 h. Dibenzyl phosphonate (8.42 mL, 38.1 mmol) was added, and the mixture was stirred at reflux for 3 h. The reaction was cooled to RT and stirred for 48 h. TEA (2.126 mL, 15.25 mmol) was added, and the mixture was concentrated. Purification by Si (0-50% EtOAc/Hex) afforded the title compound as a colorless solid. (18.6 g, 92% yield). MS (m/z) 534.1 (M+H)⁺

### Step 2: (bis(benzyloxy)phosphoryl)methanaminium Chloride

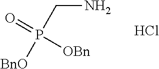 [SMILES: NCP(=O)(OCc1ccccc1)OCc1ccccc1 ; Cl]

HCl (4 M in dioxane) (40.7 mL, 163 mmol) was added to a mixture of dibenzyl ((tritylamino)methyl)phosphonate (17.39 g, 32.6 mmol) in THF (200 mL). The resulting mixture was stirred at RT for 18 h and concentrated to afford the title compound as colorless solid. (10.7 g, 100% yield). MS (m/z) 292.0 (M+H)⁺

### Step 3: Dibenzyl ((4-bromo-2-ethoxybenzamido)methyl)phosphonate

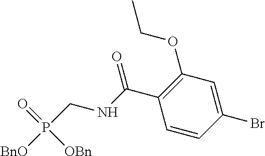 [SMILES: CCOc1cc(Br)ccc1C(=O)NCP(=O)(OCc1ccccc1)OCc1ccccc1]

To a solution containing 4-bromo-2-ethoxybenzoic acid (7.00 g, 28.6 mmol), (bis(benzyloxy)phosphoryl)methanaminium chloride (10.77 g, 32.8 mmol), EDC (8.21 g, 42.8 mmol), and 3H-[1,2,3]triazolo[4,5-b]pyridin-3-ol (3.89 g, 28.6 mmol) in DMF (143 mL) was added N-methylmorpholine (12.56 mL, 114 mmol). The reaction mixture was stirred at RT for 1 h. EtOAc was added and the organics were washed with saturated water (2×) and brine (1×). The combined washes were back extracted using EtOAc (1×). The combined extracts were dried over MgSO4, filtered, and concentrated. Purification by Si (0-100% EtOAc/Hex) afforded the title compound as a colorless solid. (4.05 g, 22% yield). MS (m/z) 518.0 (M+H)⁺

### Step 4: Dibenzyl ((2-ethoxy-4-(4,4,5,5-tetramethyl-1,3,2-dioxaborolan-2-yl)benzamido)methyl)phosphonate

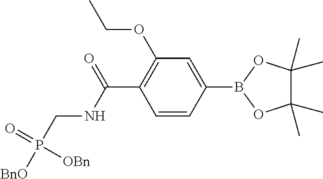 [SMILES: CCOc1cc(B2OC(C)(C)C(C)(C)O2)ccc1C(=O)NCP(=O)(OCc1ccccc1)OCc1ccccc1]

4,4,4′,4′,5,5,5′,5′-octamethyl-2,2′-bi(1,3,2-dioxaborolane) (24.45 g, 96 mmol), potassium acetate (31.5 g, 321 mmol), and PdCl2(dppf)-CH2Cl2 (2.62 g, 3.21 mmol) were added to a N₂ degassed solution of dibenzyl ((4-bromo-2-ethoxybenzamido)methyl)phosphonate (33.27 g, 64.2 mmol) in 1,4-Dioxane (321 mL). The reaction mixture was heated to 100° C. for 2 h. The mixture was cooled to RT. Water was added, and the crude mixture was filtered through a pad of Celite. The Celite was washed thoroughly with excess EtOAc (1 L). The layers were separated and additional organics were extracted from the aqueous layer with EtOAc (2×1 L). The combined organics were washed with brine (3×), dried over MgSO₄, filtered, and concentrated. Purification by Si (0-100% EtOAc/Hex) afforded the title compound as a colorless solid. (28.5 g, 79% yield). MS (m/z) 566.3 (M+H)⁺

### Step 5: Dibenzyl ((2-ethoxy-4-(5-((((R)-2-((R)-1-(N-hydroxyformamido)propyl)heptanamido)methyl)carbamoyl)furan-2-yl)benzamido)methyl)phosphonate

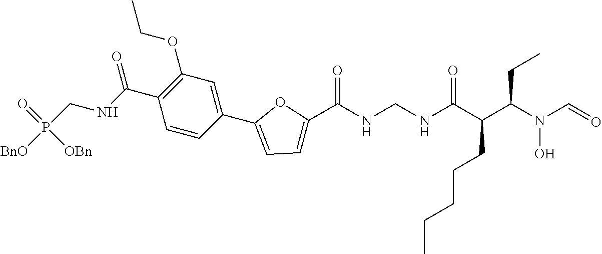 [SMILES: CCCCC[C@@H](C(=O)NCNC(=O)c1ccc(-c2ccc(C(=O)NCP(=O)(OCc3ccccc3)OCc3ccccc3)c(OCC)c2)o1)[C@@H](CC)N(O)C=O]

To a solution containing dibenzyl ((2-ethoxy-4-(4,4,5,5-tetramethyl-1,3,2-dioxaborolan-2-yl)benzamido)methyl)phosphonate (2.00 g, 3.54 mmol), 5-bromo-N—(((R)-2-((R)-1-(N-hydroxyformamido)propyl)heptanamido)methyl)furan-2-carboxamide (1.76 g, 4.07 mmol), and PdCl2(dppf)-CH2Cl2 (0.159 g, 0.195 mmol) in 1,4-Dioxane (17.69 mL) was added sodium carbonate (10.61 mL, 10.61 mmol). The resulting mixture was stirred at 70° C. for 5 h and was then heated for 18 h at 50° C. Water and EtOAc (excess) were added, and the organics were washed with water (2×) and brine (1×). The combined washes were back-extracted using EtOAc (1×). The combined organics were dried over MGSO4, filtered, and concentrated. Purification by Si (0-10% MeOH/DCM) afforded the title compound as a tan solid. (924 mg, 33% yield). MS (m/z) 791.6 (M+H)⁺

### Step 6: Dibenzyl ((4-(5-((((R)-2-((R)-1-(N-(benzoyloxy)formamido)propyl)heptanamido)methyl)carbamoyl)furan-2-yl)-2-ethoxybenzamido)methyl)phosphonate

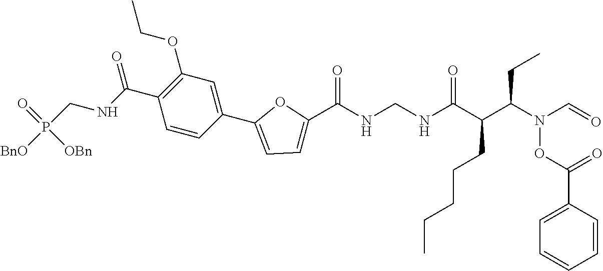 [SMILES: CCCCC[C@@H](C(=O)NCNC(=O)c1ccc(-c2ccc(C(=O)NCP(=O)(OCc3ccccc3)OCc3ccccc3)c(OCC)c2)o1)[C@@H](CC)N(C=O)OC(=O)c1ccccc1]

To a stirring solution of dibenzyl ((2-ethoxy-4-(5-((((R)-2-((R)-1-(N-hydroxyformamido)propyl)heptanamido)methyl)carbamoyl)furan-2-yl)benzamido)methyl)phosphonate (75 mg, 0.095 mmol) and benzoic anhydride (107 mg, 0.474 mmol) in MeCN (948 μl) was added TEA (66.1 μl, 0.474 mmol). The mixture was stirred for 1 h and was then concentrated to dryness. Purification by Si (0-100% EtOAc/Hex) afforded the title compound as a tan solid. (63 mg, 74% yield). MS (m/z) 895.6 (M+H)⁺

### Intermediate 153: (S)-di-tert-butyl 2-(4-(5-((((R)-2-((R)-1-(N-((2-bromo-4-methoxybenzoyl)oxy)formamido)propyl)heptanamido)methyl)carbamoyl)furan-2-yl)-2-ethoxybenzamido)succinate

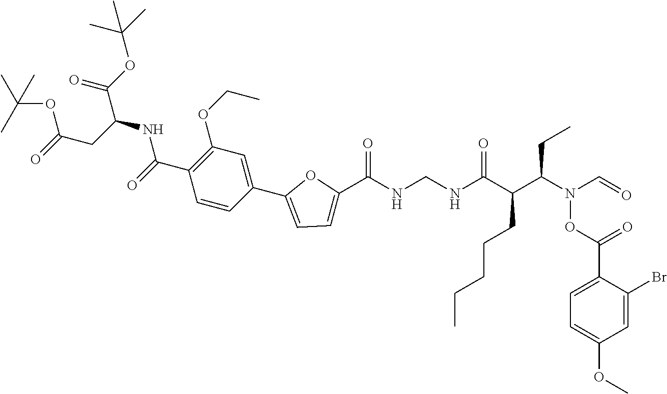 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(C=O)OC(=O)c1ccc(OC)cc1Br]

To a solution containing (S)-di-tert-butyl 2-(2-ethoxy-4-(5-((((R)-2-((R)-1-(N-hydroxyformamido)propyl)heptanamido)methyl)carbamoyl)furan-2-yl)benzamido)succinate (89.1 mg, 0.120 mmol), 2-bromo-4-methoxybenzoic acid (33.2 mg, 0.144 mmol), and HATU (68.2 mg, 0.179 mmol) in MeCN (0.7 mL) and DMF (0.7 mL) at RT was added N-methylmorpholine (0.039 mL, 0.359 mmol). The reaction mixture was stirred at RT for 1 h and then at 50° C. for 16 h. The mixture was partitioned between EtOAc and water, then the organic layer was washed with brine. It was then dried over MgSO₄, filtered, and concentrated. Purification by Si (0-100% EtOAc/Hex) afforded the title compound as a tan solid. (81 mg, 71% yield). MS (m/z) 957.9 (M+H)⁺

### Intermediate 154: (S)-di-tert-butyl 2-(4-(5-((((R)-2-((R)-1-(N-((2-chloro-4-methoxybenzoyl)oxy)formamido)propyl)heptanamido)methyl)carbamoyl)furan-2-yl)-2-ethoxybenzamido)succinate

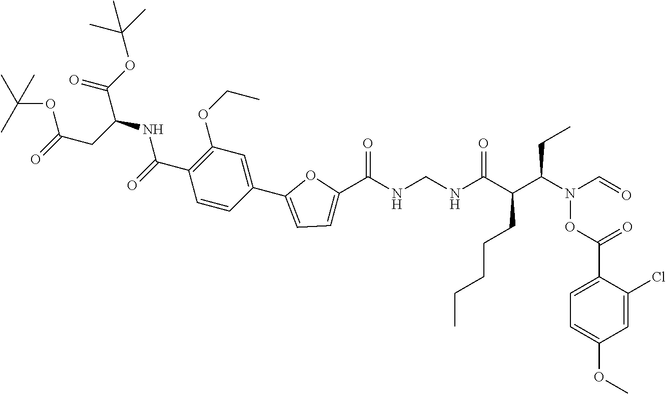 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(C=O)OC(=O)c1ccc(OC)cc1Cl]

To a solution containing (S)-di-tert-butyl 2-(2-ethoxy-4-(5-((((R)-2-((R)-1-(N-hydroxyformamido)propyl)heptanamido)methyl)carbamoyl)furan-2-yl)benzamido)succinate (89.1 mg, 0.120 mmol), 2-chloro-4-methoxybenzoic acid (26.8 mg, 0.144 mmol), and HATU (68.2 mg, 0.179 mmol) in MeCN (0.7 mL) and DMF (0.7 mL) at RT was added N-methylmorpholine (0.039 mL, 0.359 mmol). The reaction mixture was stirred at RT for 1 h and then at 50° C. for 16 h. The mixture was partitioned between EtOAc and water, then the organic layer was washed with brine. It was then dried over MgSO₄, filtered, and concentrated. Purification by Si (0-100% EtOAc/Hex) afforded the title compound as a tan solid. (88 mg, 81% yield). MS (m/z) 913.9 (M+H)⁺

### Intermediate 155: (S)-dibenzyl 2-(2-ethoxy-4-(5-((((R)-2-((R)-1-(N-((2-methyl-4-(trifluoromethyl)benzoyl)oxy)formamido)propyl)heptanamido)methyl)carbamoyl)furan-2-yl)benzamido)succinate

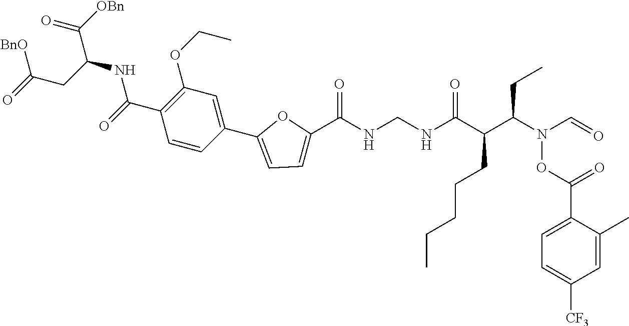 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(C(F)(F)F)cc1C]

To a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (74.4 mg, 0.092 mmol), 2-methyl-4-(trifluoromethyl)benzoic acid (22.42 mg, 0.110 mmol), and HATU (52.2 mg, 0.137 mmol) in MeCN (0.5 mL) and DMF (0.5 mL) at RT was added N-methylmorpholine (0.030 mL, 0.275 mmol). The reaction was warmed to 50° C. and stirred for 16 h. The mixture was partitioned between EtOAc and water, then the organic layer was washed with brine. It was then dried over MgSO₄, filtered, and concentrated. Purification by Si (0-100% EtOAc/Hex) afforded the title compound as a tan solid. (61 mg, 67% yield). MS (m/z) 999.5 (M+H)⁺

### Intermediate 156: (S)-2-(2-ethoxy-4-(5-((((R)-2-((R)-1-(N-((2-methyl-4-nitrosobenzoyl)oxy)formamido)propyl)heptanamido)methyl)carbamoyl)furan-2-yl)benzamido)succinic Acid and (S)-2-(4-(5-((((R)-2-((R)-1-(N-((4-amino-2-methylbenzoyl)oxy)formamido)propyl)heptanamido)methyl)carbamoyl)furan-2-yl)-2-ethoxybenzamido)succinic Acid

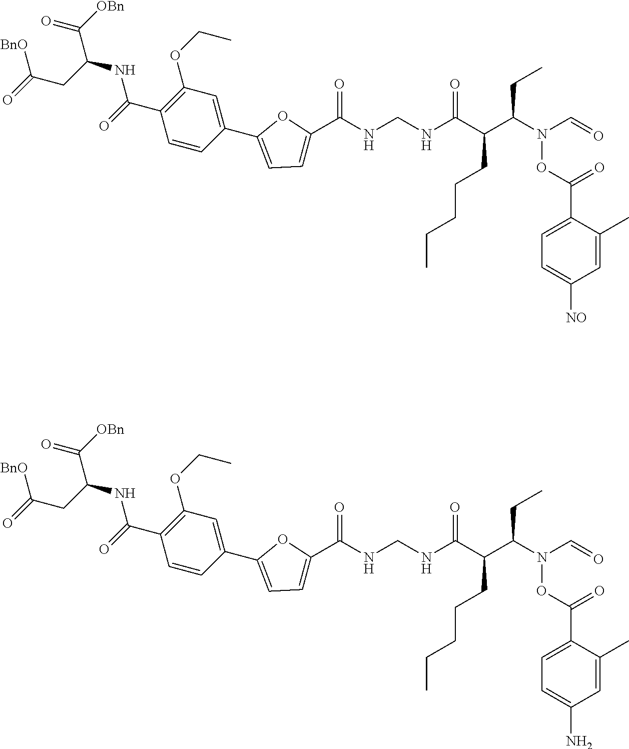

### Step 1: (S)-dibenzyl 2-(2-ethoxy-4-(5-((((R)-2-((R)-1-(N-((2-methyl-4-nitrobenzoyl)oxy)formamido)propyl)heptanamido)methyl)carbamoyl)furan-2-yl)benzamido)succinate

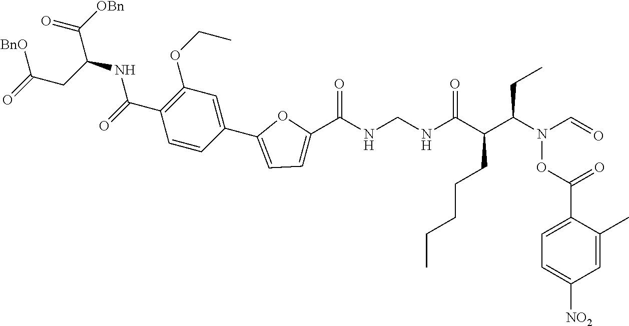 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc([N+](=O)[O-])cc1C]

To a solution containing (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (91.2 mg, 0.112 mmol), 2-methyl-4-nitrobenzoic acid (24.39 mg, 0.135 mmol), and HATU (64.0 mg, 0.168 mmol) in MeCN (0.5 mL) and DMF (0.5 mL) at RT was added N-methylmorpholine (0.037 mL, 0.337 mmol). The reaction was stirred at 50° C. for 16 h. The mixture was partitioned between EtOAc and water, then the organic layer was washed with brine. It was then dried over MgSO₄, filtered, and concentrated. Purification by Si (0-100% EtOAc/Hex) afforded the title compound. (107 mg, 98% yield). MS (m/z) 976.9 (M+H)⁺

### Step 2: (S)-2-(2-ethoxy-4-(5-((((R)-2-((R)-1-(N-((2-methyl-4-nitrosobenzoyl)oxy)formamido)propyl)heptanamido)methyl)carbamoyl)furan-2-yl)benzamido)succinic Acid and (S)-2-(4-(5-((((R)-2-((R)-1-(N-((4-amino-2-methylbenzoyl)oxy)formamido)propyl)heptanamido)methyl)carbamoyl)furan-2-yl)-2-ethoxybenzamido)succinic Acid

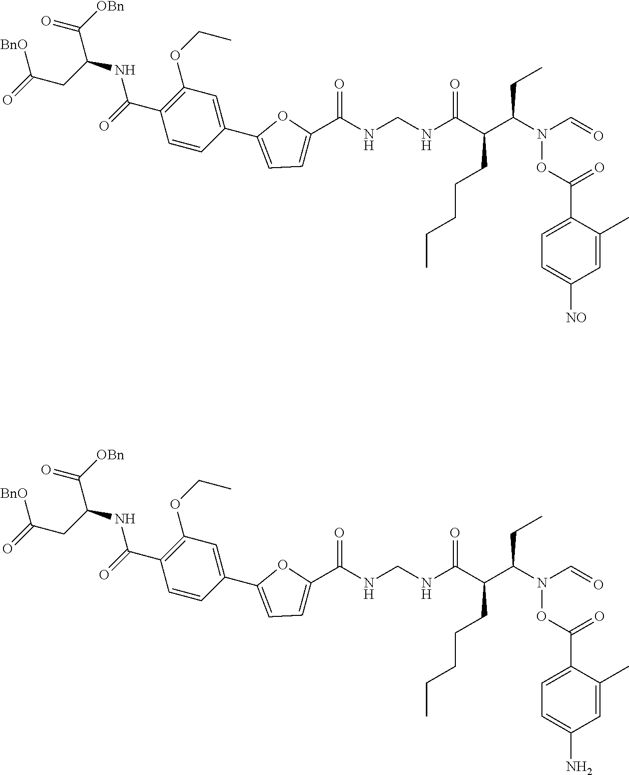

To a solution containing (S)-dibenzyl 2-(2-ethoxy-4-(5-((((R)-2-((R)-1-(N-((2-methyl-4-nitrobenzoyl)oxy)formamido)propyl)heptanamido)methyl)carbamoyl)furan-2-yl)benzamido)succinate (107 mg, 0.110 mmol) in DCM (1 mL) under N2 was added palladium on carbon (11.67 mg, 10.96 μmol) followed by EtOH (2 mL). The flask was evacuated and backfilled with N₂ twice, then evacuated and backfilled with H₂ twice. The resulting mixture was stirred at 23° C. for 20 min. The reaction was filtered through celite, washed with EtOAc and concentrated to afford the title compound as a mixture. (55 mg, 64% yield). MS (m/z) 780.6 (M+H)⁺ and (15 mg, 18% yield). MS (m/z) 766.6 (M+H)⁺

### Intermediate 157: (S)-dibenzyl 2-(4-(5-((((R)-2-((R)-1-(N-((4-(4H-1,2,4-triazol-4-yl)benzoyl)oxy)formamido)propyl)heptanamido)methyl)carbamoyl)furan-2-yl)-2-ethoxybenzamido)succinate

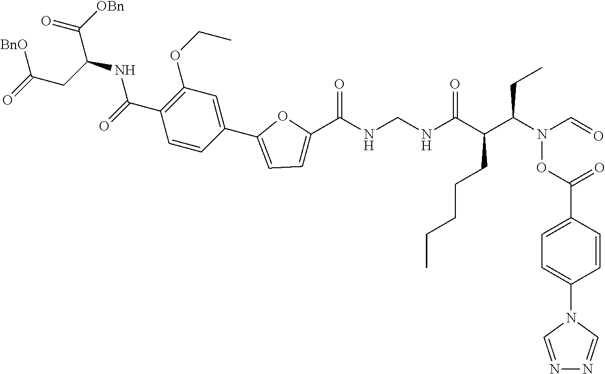 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(-n2cnnc2)cc1]

To a solution containing 4-(4H-1,2,4-triazol-4-yl)benzoic acid (25.6 mg, 0.135 mmol) and HATU (51.5 mg, 0.135 mmol) in MeCN (1 mL) was added DIPEA (0.064 mL, 0.369 mmol). The reaction was stirred for 20 min, (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (100 mg, 0.123 mmol) was added and the reaction was stirred at RT for 1 h. Water was added and the reaction was extracted with EtOAc. The organic layer was dried over Na₂SO₄ and concentrated. Purification by Si (0-% EtOAc/Hex→5% MeOH in DCM) afforded the title compound as transparent foam. (101 mg, 83% yield). MS (m/z) 984.4 (M+H)⁺

### Intermediate 158: (S)-dibenzyl 2-(4-(5-((((R)-2-((R)-1-(N-((3-(1H-pyrrol-1-yl)benzoyl)oxy)formamido)propyl)heptanamido)methyl)carbamoyl)furan-2-yl)-2-ethoxybenzamido)succinate

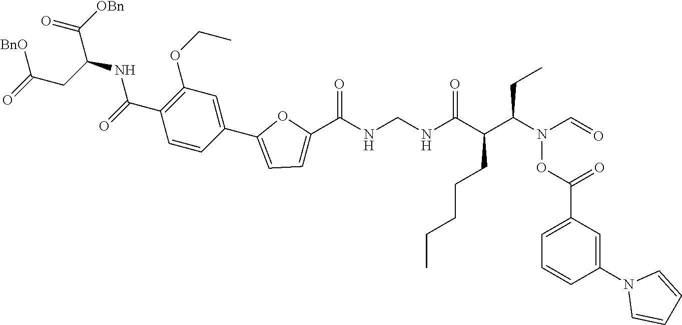 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1cccc(-n2cccc2)c1]

To a solution containing 3-(1H-pyrrol-1-yl)benzoic acid (25.3 mg, 0.135 mmol) and HATU (51.5 mg, 0.135 mmol) in MeCN (1 mL) was added DIPEA (0.064 mL, 0.369 mmol). The reaction was stirred for 20 min, (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (100 mg, 0.123 mmol) was added and the reaction was stirred at RT for 18 h. Water was added and the reaction was extracted with EtOAc. The organic layer was dried over Na₂SO₄ and concentrated. Purification by Si (0-85% EtOAc/Hex) followed by purification by reverse phase Si-C18 (0-90% water with 0.1% TFA/MeCN) afforded the title compound as an off white solid. (76 mg, 63% yield). MS (m/z) 982.4 (M+H)⁺

### Intermediate 159: (S)-dibenzyl 2-(2-ethoxy-4-(5-((((R)-2-((R)-1-(N-((4-(N-methylmethylsulfonamido)benzoyl)oxy)formamido)propyl)heptanamido)methyl)carbamoyl)furan-2-yl)benzamido)succinate

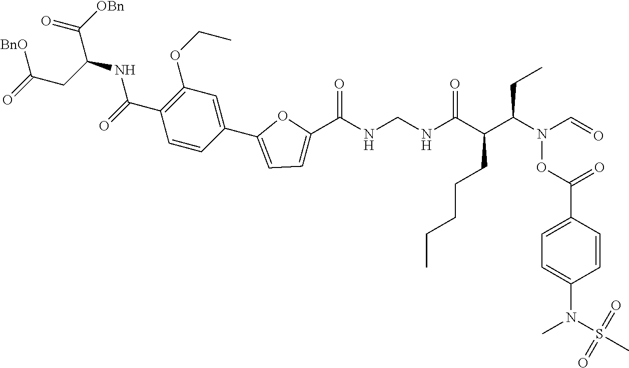 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(N(C)S(C)(=O)=O)cc1]

To a solution containing 4-(N-methylmethylsulfonamido)benzoic acid (31.0 mg, 0.135 mmol) and HATU (51.5 mg, 0.135 mmol) in MeCN (1 mL) was added DIPEA (0.064 mL, 0.369 mmol). The reaction was stirred for 20 min, (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (100 mg, 0.123 mmol) was added and the reaction was stirred at RT for 1 h. Water was added and the reaction was extracted with EtOAc. The organic layer was dried over Na₂SO₄ and concentrated. Purification by Si (0-85% EtOAc/Hex) followed by purification by reverse phase Si-C18 (0-90% water with 0.1% TFA/MeCN) afforded the title compound as an off white solid. (85 mg, 68% yield). MS (m/z) 512.8 (M/2+H)⁺

### Intermediate 160: (S)-dibenzyl 2-(4-(5-((((R)-2-((R)-1-(N-((4-(1H-pyrazol-1-yl)benzoyl)oxy)formamido)propyl)heptanamido)methyl)carbamoyl)furan-2-yl)-2-ethoxybenzamido)succinate

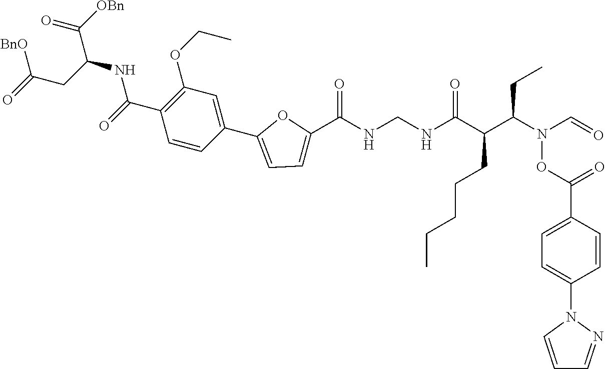 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(-n2cccn2)cc1]

To a solution containing 4-(1H-pyrazol-1-yl)benzoic acid (25.5 mg, 0.135 mmol) and HATU (51.5 mg, 0.135 mmol) in MeCN (1 mL) was added DIPEA (0.064 mL, 0.369 mmol). The reaction was stirred for 20 min, (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (100 mg, 0.123 mmol) was added and the reaction was stirred at RT for 1 h. Water was added and the reaction was extracted with EtOAc. The organic layer was dried over Na₂SO₄ and concentrated. Purification by Si (0-90% EtOAc/Hex) followed by purification by reverse phase Si-C18 (0-90% water with 0.1% TFA/MeCN) afforded the title compound as a colorless solid. (75 mg, 62% yield). MS (m/z) 983.4 (M+H)⁺

### Intermediate 161: (S)-dibenzyl 2-(4-(5-((((R)-2-((R)-1-(N-((4-(1H-imidazol-1-yl)benzoyl)oxy)formamido)propyl)heptanamido)methyl)carbamoyl)furan-2-yl)-2-ethoxybenzamido)succinate

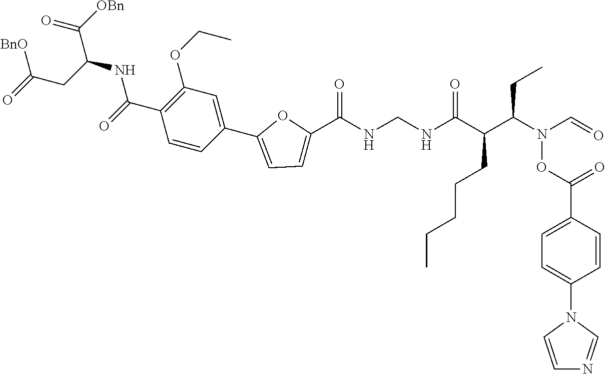 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(-n2ccnc2)cc1]

To a solution containing 4-(1H-imidazol-1-yl)benzoic acid (25.5 mg, 0.135 mmol) and HATU (51.5 mg, 0.135 mmol) in MeCN (615 μl) was added DIPEA (64.5 μl, 0.369 mmol) and stirred for at RT for 30 mins. (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (100 mg, 0.123 mmol) was added and the reaction was stirred at RT for 2 h. Water was added and the reaction was extracted with EtOAc. The organic layer was dried over Na₂SO₄ and concentrated. Purification by Si (0-100% EtOAc/Hex) afforded the title compound as a tan solid. (112 mg, 93% yield). MS (m/z) 983.4 (M+H)⁺

### Intermediate 162: Dibenzyl ((2-ethoxy-4-(5-((((R)-2-((R)-1-(N-((4-methoxy-2-methylbenzoyl)oxy)formamido)propyl)heptanamido)methyl)carbamoyl)furan-2-yl)benzamido)methyl)phosphonate

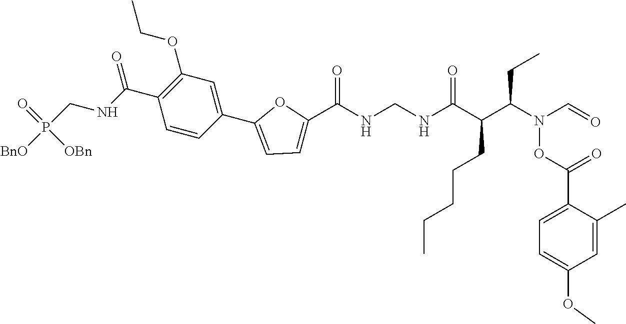 [SMILES: CCCCC[C@@H](C(=O)NCNC(=O)c1ccc(-c2ccc(C(=O)NCP(=O)(OCc3ccccc3)OCc3ccccc3)c(OCC)c2)o1)[C@@H](CC)N(C=O)OC(=O)c1ccc(OC)cc1C]

To a solution containing 4-methoxy-2-methylbenzoic acid (18.91 mg, 0.114 mmol) in MeCN (474 μl) was added N-methylmorpholine (31.3 μl, 0.285 mmol) and HATU (43.3 mg, 0.114 mmol). The mixture was stirred for 15 min and was then added to a stirring solution of dibenzyl ((2-ethoxy-4-(5-((((R)-2-((R)-1-(N-hydroxyformamido)propyl)heptanamido)methyl)carbamoyl)furan-2-yl)benzamido)methyl)phosphonate (75 mg, 0.095 mmol) in DMF (474 μl). The resulting mixture was stirred for 1 h at 50° C. and was then concentrated to dryness. Purification by Si (0-100% EtOAc/Hex) afforded the title compound as a tan solid. (42 mg, 20% yield). MS (m/z) 939.8 (M+H)⁺

### Intermediate 163: Dibenzyl ((2-ethoxy-4-(5-((((R)-2-((R)-1-(N-((2-methyl-4-(morpholinomethyl)benzoyl)oxy)formamido)propyl)heptanamido)methyl)carbamoyl)furan-2-yl)benzamido)methyl)phosphonate

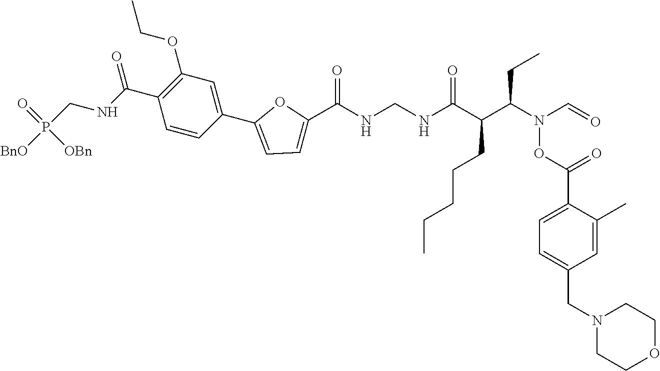 [SMILES: CCCCC[C@@H](C(=O)NCNC(=O)c1ccc(-c2ccc(C(=O)NCP(=O)(OCc3ccccc3)OCc3ccccc3)c(OCC)c2)o1)[C@@H](CC)N(C=O)OC(=O)c1ccc(CN2CCOCC2)cc1C]

To a solution containing 4-(4-carboxy-3-methylbenzyl)morpholin-4-ium chloride (30.9 mg, 0.114 mmol) in MeCN (474 μl) was added N-methylmorpholine (41.7 μl, 0.379 mmol) and HATU (43.3 mg, 0.114 mmol). The mixture was stirred for 15 min and was then added to a stirring solution of dibenzyl ((2-ethoxy-4-(5-((((R)-2-((R)-1-(N-hydroxyformamido)propyl)heptanamido)methyl)carbamoyl)furan-2-yl)benzamido)methyl)phosphonate (75 mg, 0.095 mmol) in DMF (474 μl). The resulting mixture was stirred for 1 h at 50° C. and was then concentrated to dryness. Purification by Si (0-100% EtOAc/Hex) afforded the title compound as a tan solid. (60 mg, 46% yield). MS (m/z) 505.2 ((M+2)/2)+

### Intermediate 164: Dibenzyl ((2-ethoxy-4-(5-((((R)-2-((R)-1-(N-((4-methoxybenzoyl)oxy)formamido)propyl)heptanamido)methyl)carbamoyl)furan-2-yl)benzamido)methyl)phosphonate

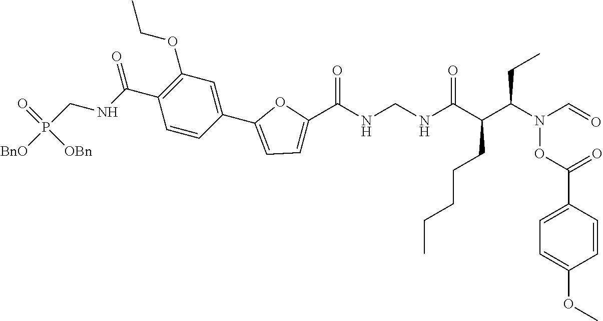 [SMILES: CCCCC[C@@H](C(=O)NCNC(=O)c1ccc(-c2ccc(C(=O)NCP(=O)(OCc3ccccc3)OCc3ccccc3)c(OCC)c2)o1)[C@@H](CC)N(C=O)OC(=O)c1ccc(OC)cc1]

To a solution containing 4-methoxybenzoic acid (17.31 mg, 0.114 mmol) in MeCN (474 μl) was added N-methylmorpholine (31.3 μl, 0.285 mmol) and HATU (43.3 mg, 0.114 mmol). The mixture was stirred for 15 min and was then added to a stirring solution of dibenzyl ((2-ethoxy-4-(5-((((R)-2-((R)-1-(N-hydroxyformamido)propyl)heptanamido)methyl)carbamoyl)furan-2-yl)benzamido)methyl)phosphonate (75 mg, 0.095 mmol) in DMF (474 μl). The resulting mixture was stirred for 1 h at 50° C. and was then concentrated to dryness. Purification by Si (0-100% EtOAc/Hex) afforded the title compound as a tan solid. (64 mg, 73% yield). MS (m/z) 925.7 (M+H)⁺

### Intermediate 165: (S)-di-tert-butyl 2-(4-(5-((((R)-2-((R)-1-(N-((4-((tert-butoxycarbonyl)(methyl)amino)benzoyl)oxy)formamido)propyl)heptanamido)methyl)carbamoyl)furan-2-yl)-2-ethoxybenzamido)succinate

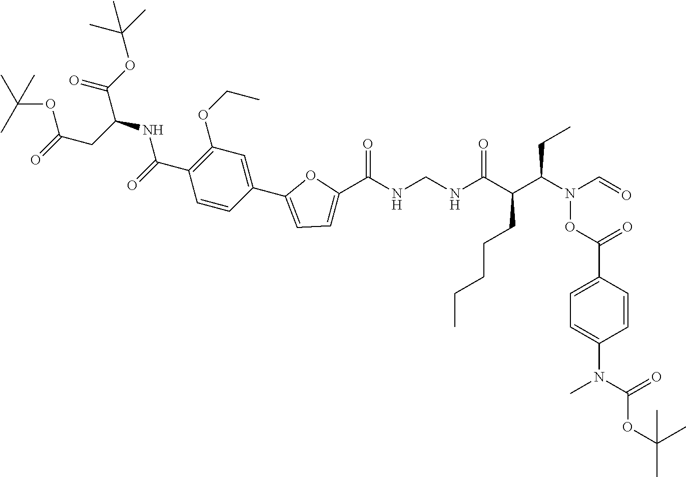 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(C=O)OC(=O)c1ccc(N(C)C(=O)OC(C)(C)C)cc1]

To a solution containing 4-((tert-butoxycarbonyl)(methyl)amino)benzoic acid (76 mg, 0.302 mmol) in MeCN (0.75 mL) and DMF (0.75 mL) was added N-methylmorpholine (66 μl, 0.604 mmol) and HATU (138 mg, 0.362 mmol). The mixture was stirred for 15 min and (S)-di-tert-butyl 2-(2-ethoxy-4-(5-((((R)-2-((R)-1-(N-hydroxyformamido)propyl)heptanamido)methyl)carbamoyl)furan-2-yl)benzamido)succinate (150 mg, 0.201 mmol) was added. The reaction was stirred for 18 h at RT and concentrated to remove solvent. The reaction was diluted each with DCM and water (5 mL each). The organic phase was passed through a phase separator. Purification by Si (0-100% EtOAc/Hex) afforded the title compound as a colorless solid. (167 mg, 83% yield). MS (m/z) 978.7 (M+H)⁺

### Intermediate 166: (S)-di-tert-butyl 2-(4-(5-((((R)-2-((R)-1-(N-((4-((tert-butoxycarbonyl)amino)benzoyl)oxy)formamido)propyl)heptanamido)methyl)carbamoyl)furan-2-yl)-2-ethoxybenzamido)succinate

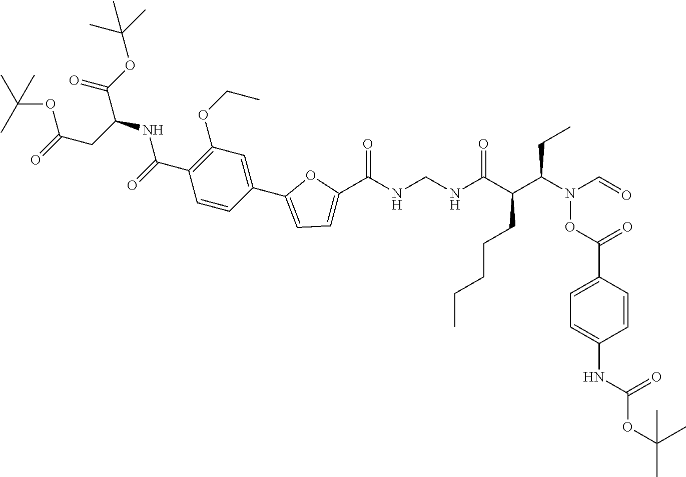 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(C=O)OC(=O)c1ccc(NC(=O)OC(C)(C)C)cc1]

To a solution containing 4-((tert-butoxycarbonyl)amino)benzoic acid (71.7 mg, 0.302 mmol) in MeCN (0.75 mL) and DMF (0.75 mL) was added N-methylmorpholine (66 μl, 0.604 mmol) and HATU (138 mg, 0.362 mmol). The mixture was stirred for 15 min and (S)-di-tert-butyl 2-(2-ethoxy-4-(5-((((R)-2-((R)-1-(N-hydroxyformamido)propyl)heptanamido)methyl)carbamoyl)furan-2-yl)benzamido)succinate (150 mg, 0.201 mmol) was added. The reaction was stirred for 48 h at RT and concentrated down to remove solvent. The reaction was diluted with DCM and water (5 mL each). The organic phase was passed through a phase separator. Purification by Si (0-100% EtOAc/Hex) afforded the title compound as a colorless solid. (135 mg, 68% yield). MS (m/z) 964.7 (M+H)⁺

### Intermediate 167: (S)-di-tert-butyl 2-(4-(5-((((R)-2-((R)-1-(N-((4-(((tert-butoxycarbonyl)amino)methyl)benzoyl)oxy)formamido)propyl)heptanamido)methyl)carbamoyl)furan-2-yl)-2-ethoxybenzamido)succinate

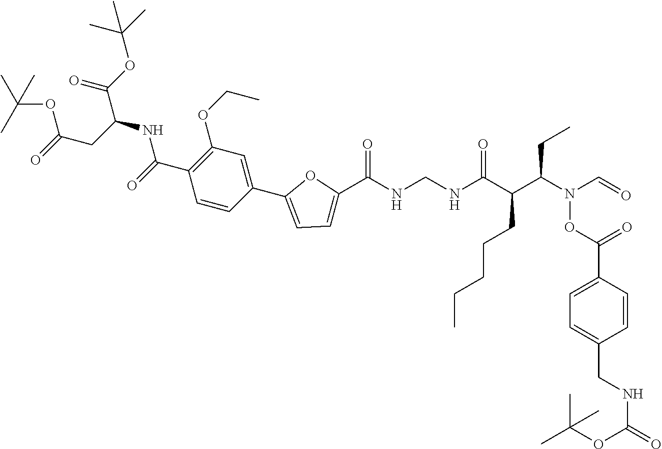 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(C=O)OC(=O)c1ccc(CNC(=O)OC(C)(C)C)cc1]

To a solution containing 4-(((tert-butoxycarbonyl)amino)methyl)benzoic acid (76 mg, 0.302 mmol) in MeCN (0.75 mL) and DMF (0.75 mL) was added N-methylmorpholine (66 μl, 0.604 mmol) and HATU (138 mg, 0.362 mmol). The mixture was stirred for 15 min and (S)-di-tert-butyl 2-(2-ethoxy-4-(5-((((R)-2-((R)-1-(N-hydroxyformamido)propyl)heptanamido)methyl)carbamoyl)furan-2-yl)benzamido)succinate (150 mg, 0.201 mmol) was added. The reaction was stirred for 48 h at RT and concentrated down to remove solvent. The reaction was diluted with DCM and water (5 mL each). The organic phase was passed through a phase separator. Purification by Si (0-100% EtOAc/Hex) afforded the title compound as a colorless solid. (77 mg, 38% yield). MS (m/z) 978.7 (M+H)⁺

### Intermediate 168: (S)-dibenzyl 2-(4-(5-((((R)-2-((R)-1-(N-((4-(2-(2-(benzyloxy)ethoxy)ethoxy)benzoyl)oxy)formamido)propyl)heptanamido)methyl)carbamoyl)furan-2-yl)-2-ethoxybenzamido)succinate

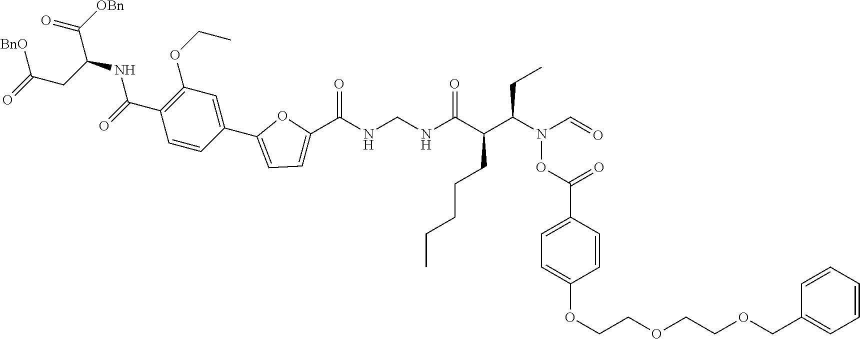 [SMILES: CCCCC[C@@H](C(=O)NCNC(=O)c1ccc(-c2ccc(C(=O)N[C@@H](CC(=O)OCc3ccccc3)C(=O)OCc3ccccc3)c(OCC)c2)o1)[C@@H](CC)N(C=O)OC(=O)c1ccc(OCCOCCOCc2ccccc2)cc1]

To a solution containing 4-(2-(2-(benzyloxy)ethoxy)ethoxy)benzoic acid (218 mg, 0.689 mmol) and hunig's base (361 μl, 2.067 mmol) in MeCN (2756 μl) was added HATU (262 mg, 0.689 mmol). The reaction was stirred at rt for 15 min. (S)-dibenzyl 2-(2-ethoxy-4-(5-((((R)-2-((R)-1-(N-hydroxyformamido)propyl)heptanamido)methyl)carbamoyl)furan-2-yl)benzamido)succinate (560 mg, 0.689 mmol) was added, and the reaction was stirred overnight. The reaction was diluted with EtOAc and quenched with water. After stirring for 20 min the layers were separated. The organics were washed with water (1×1 mL) and brine (1×10 mL). The organics were then dried over MgSO4, filtered, and concentrated to afford the title compound as a colorless foam. (766 mg, 100% yield).

### Example 1

### (S)-2-(4-(5-((((R)-2-((R)-1-(N-(benzoyloxy)formamido)propyl)heptanamido)methyl)carbamoyl)furan-2-yl)-2-ethoxybenzamido)succinic Acid

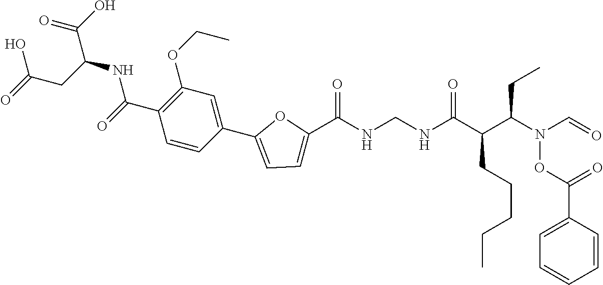 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccccc1]

Pd/C (9.98 mg, 9.38 μmol) was added to a stirring RT mixture of (S)-dibenzyl 2-(4-(5-((((R)-2-((R)-1-(N-(benzoyloxy)formamido)propyl)heptanamido)methyl)carbamoyl)furan-2-yl)-2-ethoxybenzamido)succinate (86 mg, 0.094 mmol) in DCM (188 μl). MeOH (750 μl) was added, and the mixture was evacuated and backfilled with N₂ (3×). The mixture was then evacuated and backfilled with H2 (balloon). The resulting mixture was stirred for 1 h under hydrogen. The mixture was then filtered through a syringe filter, washed with MeOH and then concentrated to afford the title compound as a colorless solid. (68.3 mg, 94% yield)

Examples 2-135 were prepared from the indicated intermediate by methods analogous to those described for Example 1 from the Intermediates listed in the table below.

| Ex. | Name | Structure | Intermediate |
|---|---|---|---|
| 2 | (3-(5-((((R)-2-((R)-1- (N- (benzoyloxy) formamido) propyl)heptanamido) methyl)carbamoyl) furan-2-yl)-5- ethoxyphenyl) phosphonic acid | 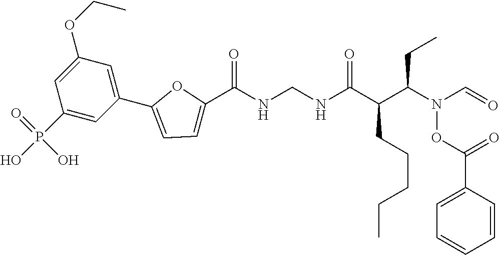 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccccc1] | dibenzyl (3-(5- ((((R)-2-((R)- 1-(N- (benzoyloxy) formamido) propyl) heptanamido) methyl) carbamoyl) furan-2- yl)-5-ethoxy phenyl) phosphonate |
| 3 | (3-ethoxy-5-(5-((((R)- 2-((R)-1-(N-(2- phenylacetoxy) formamido)propyl) heptanamido)methyl) carbamoyl) furan-2- yl)phenyl)phosphonic acid | 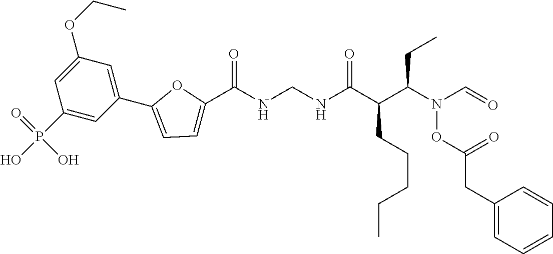 [SMILES: CCCCC[C@@H](C(=O)NCNC(=O)c1ccc(-c2cc(OCC)cc(P(=O)(O)O)c2)o1)[C@@H](CC)N(C=O)OC(=O)Cc1ccccc1] | dibenzyl (3- ethoxy-5- (5-((((R)- 2-((R)-1-(N-(2- phenylacetoxy) formamido) propyl) heptanamido) methyl) carbamoyl) furan-2- yl)phenyl) phosphonate |
| 4 | (S)-2-(2-ethoxy-4-(5- ((((R)-2-((R)-1-(N-(2- phenylacetoxy) formamido)propyl) heptanamido)methyl) carbamoyl)furan-2- yl)benzamido) succinic acid | 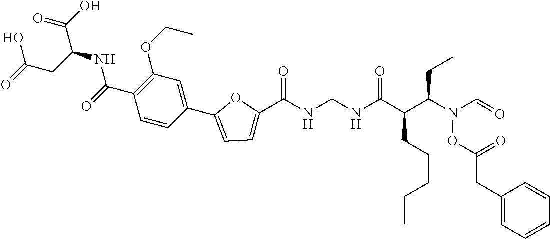 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)Cc1ccccc1] | (S)-dibenzyl 2-(2-ethoxy-4- (5-((((R)- 2-((R)-1-(N- (2-phenylacetoxy) formamido) propyl) heptanamido) methyl) carbamoyl) furan-2-yl) benzamido) succinate |
| 5 | (S)-2-(2-ethoxy-4- (5-((((R)-2-((R)-1- (N-(pivaloyloxy) formamido)propyl) heptanamido) methyl)carbamoyl) furan-2-yl)benzamido) succinic acid | 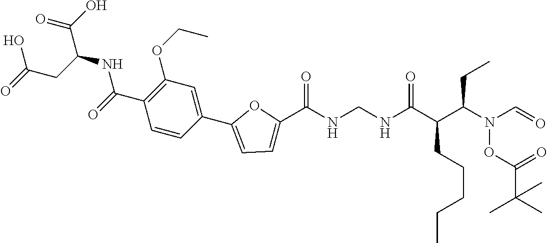 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)C(C)(C)C] | (S)-dibenzyl 2-(2- ethoxy-4-(5- ((((R)-2-((R)-1- (N-(pivaloyloxy) formamido) propyl) heptanamido) methyl) carbamoyl) furan-2- yl)benzamido) succinate |
| 6 | (S)-2-(2- (carboxymethoxy)-4- (5-((((R)-2-((R)-1- (N-((2- hydroxybenzoyl) oxy)formamido) propyl) heptanamido)methyl) carbamoyl)furan-2- yl)benzamido) succinic acid | 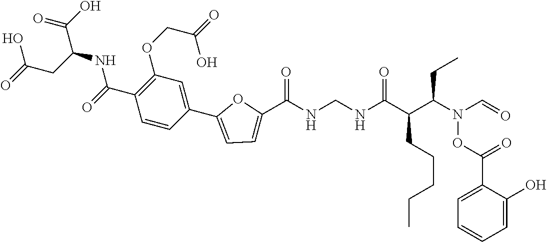 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccccc1O] | (S)-dibenzyl 2-(2- (2-(benzyloxy)-2- oxoethoxy)-4-(5- ((((R)-2-((R)-1- (N-((2-(benzyl oxy)benzoyl)oxy) formamido) propyl) heptanamido) methyl) carbamoyl)furan- 2-yl)benzamido) succinate |
| 7 | (S)-2-(4-(5-((((R)-2- ((R)-1-(N-(benzo- yloxy)formamido) propyl)heptanamido) methyl)carbamoyl) furan-2-yl)-2- (carboxymethoxy) benzamido)succinic acid | 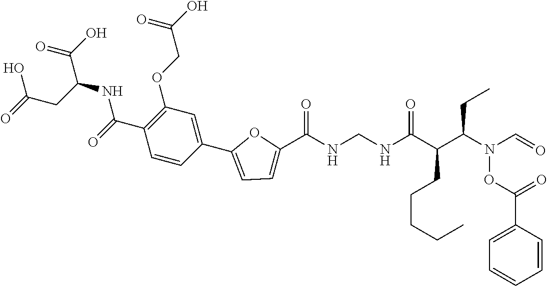 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccccc1] | (S)-dibenzyl 2-(4- (5-((((R)-2-((R)- 1-(N- (benzoyloxy) formamido) propyl) heptanamido) methyl) carbamoyl)furan- 2-yl)-2-(2- (benzyloxy)-2- oxoethoxy) benzamido) succinate |
| 8 | (S)-2-(2-(carboxy- methoxy)-4-(5-((((R)- 2-((R)-1-(N-((2- methylbenzoyl) oxy)formamido) propyl)heptanamido) methyl)carbamoyl) furan-2-yl) benzamido) succinic acid | 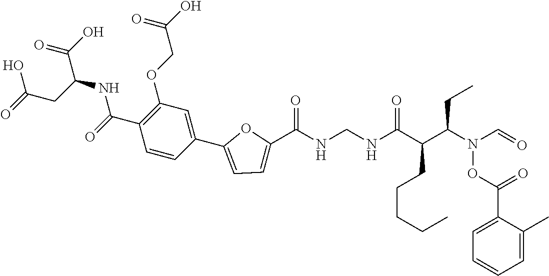 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccccc1C] | (S)-dibenzyl 2-(2- (2-(benzyloxy)- 2-oxoethoxy)-4- (5-((((R)-2-((R)- 1-(N-((2- methylbenzoyl) oxy)formamido) propyl) heptanamido) methyl) carbamoyl) furan-2-yl) benzamido) succinate |
| 9 | (3-ethoxy-5-(5- (((4R,5R)-5-ethyl-6- formyl-11,11- dimethyl- 3,10-dioxo-4- pentyl-7,9-dioxa- 2,6-diazadodecyl) carbamoyl)furan-2- yl)phenyl)phosphonic acid | 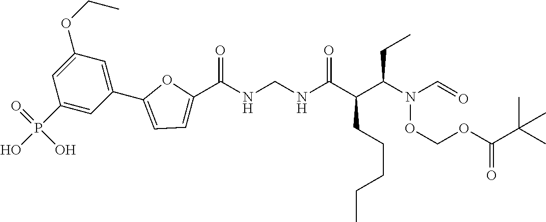 [SMILES: CCCCC[C@@H](C(=O)NCNC(=O)c1ccc(-c2cc(OCC)cc(P(=O)(O)O)c2)o1)[C@@H](CC)N(C=O)OCOC(=O)C(C)(C)C] | (4R,5R)-10-(5-(3- (bis(benzyloxy) phosphoryl)-5- ethoxy phenyl) furan-2-yl)- 4-ethyl-3-formyl- 6,10-dioxo-5- pentyl- 2-oxa-3,7,9- triazadecyl pivalate |
| 10 | (3-ethoxy-5-(5- ((((R)-2-((R)-1-(N- ((phosphonooxy) methoxy)formamido) propyl)heptanamido) methyl)carbamoyl) furan-2-yl) phenyl) phosphonic acid | 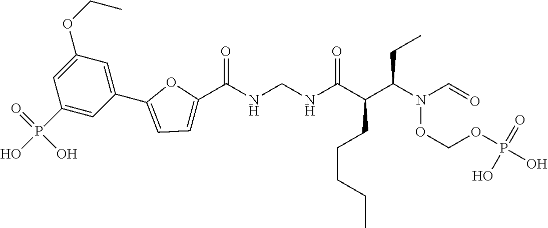 [SMILES: CCCCC[C@@H](C(=O)NCNC(=O)c1ccc(-c2cc(OCC)cc(P(=O)(O)O)c2)o1)[C@@H](CC)N(C=O)OCOP(=O)(O)O] | benzyl ((4R,5R)- 10-(5-(3- (bis(benzyloxy) phosphoryl)-5- ethoxyphenyl) furan-2-yl)-4- ethyl-3-formyl- 6,10-dioxo- 5-pentyl-2-oxa- 3,7,9-triazadecyl) hydrogen phosphate |
| 11 | (S)-2-(2- (carboxymethoxy)-4- (5-((((R)-2-((R)-1-(N- ((phosphonooxy) methoxy)formamido) propyl) heptanamido)methyl) carbamoyl)furan-2- yl)benzamido) succinic acid | 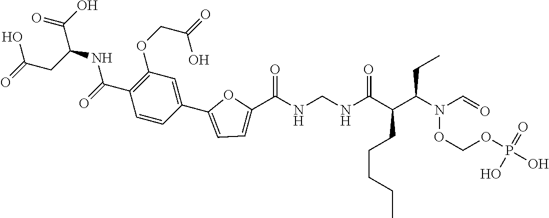 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OCOP(=O)(O)O] | (2S)-dibenzyl 2-(4-(5-((((2R)- 2-((1R)-1-(N- ((((benzyloxy) (hydroxy) phosphoryl) oxy)methoxy) formamido) propyl) heptanamido) methyl) carbamoyl) furan-2-yl)-2-(2- (benzyloxy)-2- |
| | | | oxoethoxy) |
| | | | benzamido) |
| | | | succinate |
| 12 | (S)-2-(4-(5-((((R)-2- ((R)-1-(N-((2- acetoxybenzoyl)oxy) formamido)propyl) heptanamido) methyl)carbamoyl) furan-2-yl)-2- (carboxymethoxy) benzamido) succinic acid | 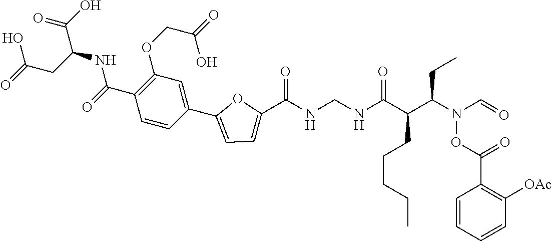 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccccc1OC(C)=O] | (S)-dibenzyl 2-(4- (5-((((R)-2-((R)- 1-(N-((2- acetoxybenzoyl) oxy)formamido) propyl) heptanamido) methyl) carbamoyl) furan-2-yl)-2-(2- (benzyloxy)-2- oxoethoxy) benzamido) succinate |
| 13 | (S)-2-(2- (carboxymethoxy)-4- (5-((((R)-2-((R)-1-(N- (nicotinoyloxy) formamido)propyl) heptanamido)methyl) carbamoyl) furan-2- yl)benzamido) succinic acid | 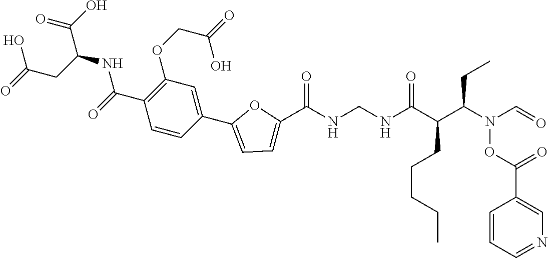 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1cccnc1] | (S)-dibenzyl 2-(2- (2-(benzyloxy)-2- oxoethoxy)-4-(5- ((((R)-2-((R)-1- (N- (nicotinoyloxy) formamido) propyl) heptanamido) methyl) carbamoyl) furan-2- yl)benzamido) succinate |
| 14 | (S)-2-(2-(carboxy methoxy)-4-(5- (((4R,5R)-5-ethyl-6- formyl-10,10- dimethyl-3,8-dioxo- 4-pentyl-7,9-dioxa- 2,6-diazaundecyl) carbamoyl)furan-2- yl)benzamido) succinic acid | 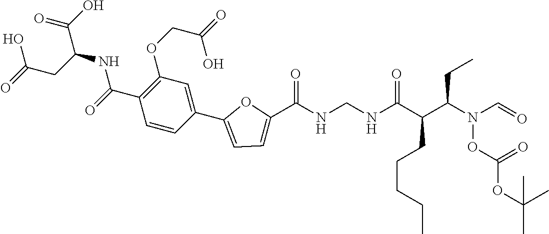 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)OC(C)(C)C] | (S)-dibenzyl 2-(2- (2-(benzyloxy)-2- oxoethoxy)-4-(5- (((4R,5R)-5- ethyl- 6-formyl-10,10- dimethyl-3,8- dioxo-4-pentyl- 7,9-dioxa-2,6- diazaundecyl) carbamoyl) furan-2- yl)benzamido) succinate |
| 16 | (3-ethoxy-5-(5- ((((R)-2-((R)-1-(N- ((2-hydroxybenzoyl) oxy)formamido) propyl)heptanamido) methyl)carbamoyl) furan-2-yl)phenyl) phosphonic acid | 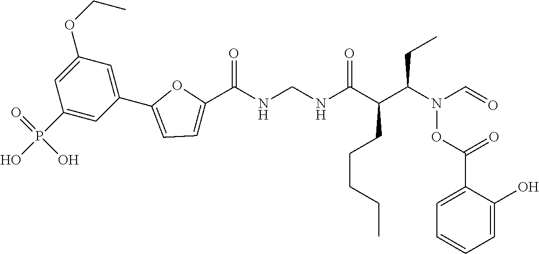 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccccc1O] | dibenzyl (3-(5- ((((R)-2-((R)- 1-(N- ((2-(benzyloxy) benzoyl)oxy) formamido) propyl) heptanamido) methyl) carbamoyl) furan-2-yl)-5- ethoxyphenyl) phosphonate |
| 17 | (S)-2-(4-(5-((((R)-2- ((R)-1-(N-((3- carboxybenzoyl)oxy) formamido)propyl) heptanamido)methyl) carbamoyl)furan-2- yl)-2- (carboxymethoxy) benzamido)succinic acid | 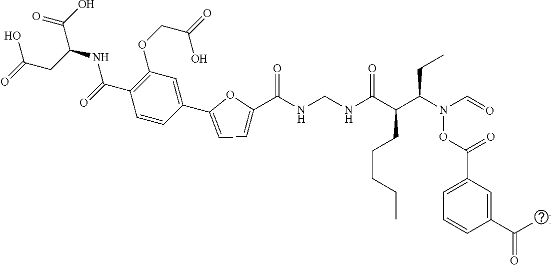 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1cccc(C(=O)O)c1] | 3-((4R,5R)-10-(5- (3-(2-(benzyloxy)- 2-oxoethoxy)-4- (((S)-1,4- bis(benzyloxy)- 1,4-dioxobutan-2- yl)carbamoyl) phenyl)furan- 2-yl)- 4-ethyl-3-formyl- 6,10-dioxo-5- pentyl-2-oxa- 3,7,9- triazadecan-1- oyl)benzoic acid |
| 18 | (S)-2-(2- (carboxymethoxy)-4- (5-((((R)-2-((R)-1-(N- ((1-methylcyclo- propanecarbonyl)oxy) formamido)propyl) heptanamido)methyl) carbamoyl)furan-2- yl)benzamido) succinic acid | 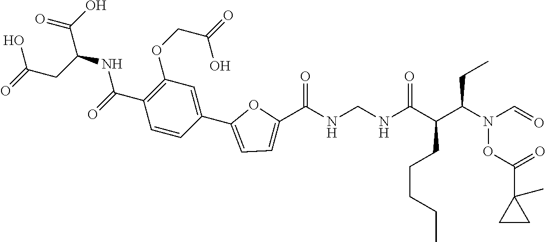 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)C1(C)CC1] | |
| 19 | (2S)-2-(2- (carboxymethoxy)-4- (5-((((2R)-2-((1R)-1- (N-((2- methylcyclopro- panecarbonyl)oxy) formamido)propyl) heptanamido)methyl) carbamoyl)furan-2- yl)benzamido) succinic acid | 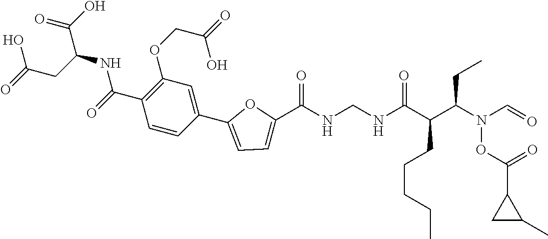 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)C1CC1C] | (2S)-dibenzyl 2-(2- (2-(benzyloxy)-2- oxoethoxy)-4-(5- ((((2R)-2-((1R)-1- (N-((2- methylcyclo- propanecarbonyl) oxy)formamido) propyl) heptanamido) methyl) carbamoyl)furan- 2-yl)benzamido) succinate |
| 20 | (S)-2-(2- (carboxymethoxy)-4- (5-((((R)-2-((R)-1-(N- ((4- methoxybenzoyl)oxy) formamido) propyl)heptanamido) methyl)carbamoyl) furan-2- yl)benzamido) succinic acid | 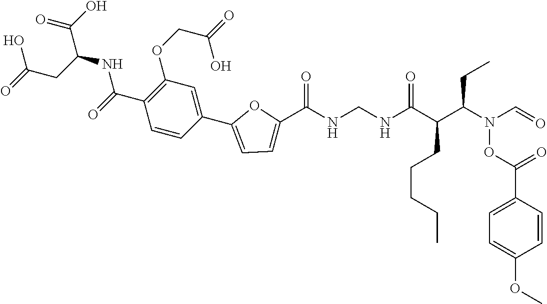 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccc(OC)cc1] | (S)-dibenzyl 2-(2- (2-(benzyloxy)-2- oxoethoxy)-4-(5- ((((R)-2-((R)-1- (N-((4- methoxybenzoyl) oxy)formamido) propyl) heptanamido) methyl) carbamoyl) furan-2- yl)benzamido) succinate |
| 21 | (S)-2-(2- (carboxymethoxy)-4- (5-(((6R,7R)-6-ethyl- 5-formyl-3,8-dioxo-7- pentyl-4-oxa-2,5,9- triazadecan-10- yl)carbamoyl)furan-2- yl)benzamido) succinic acid | 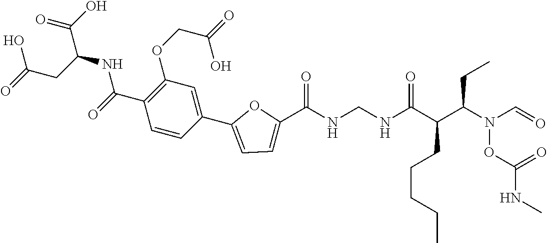 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)NC] | (S)-dibenzyl 2-(2- (2-(benzyloxy)-2- oxoethoxy)-4-(5- (((6R,7R)-6-ethyl- 5-formyl-3,8- dioxo- 7-pentyl-4-oxa- 2,5,9-triazadecan- 10-yl)carbamoyl) furan-2- yl)benzamido) succinate |
| 22 | (S)-2-(4-(5-((((R)-2- ((R)-1-(N-((2- aminobenzoyl)oxy) formamido)propyl) heptanamido)methyl) carbamoyl) furan-2-yl)-2- (carboxymethoxy) benzamido) succinic acid | 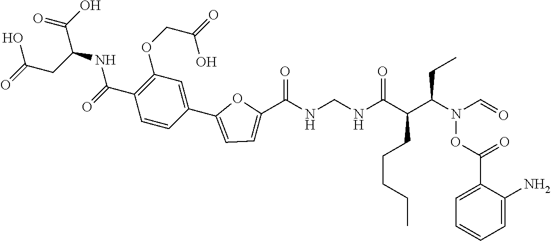 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccccc1N] | (S)-dibenzyl 2- (4-(5-((((R)- 2-((R)-1-(N-((2- aminobenzoyl) oxy) formamido) propyl) heptanamido) methyl) carbamoyl) furan-2-yl)-2-(2- (benzyloxy)- 2-oxoethoxy) benzamido) succinate |
| 23 | (S)-2-(2- (carboxymethoxy)-4- (5-((((R)-2-((R)-1- (N-((2-(methylamino) benzoyl)oxy) formamido)propyl) heptanamido) methyl)carbamoyl) furan-2- yl)benzamido) succinic acid | 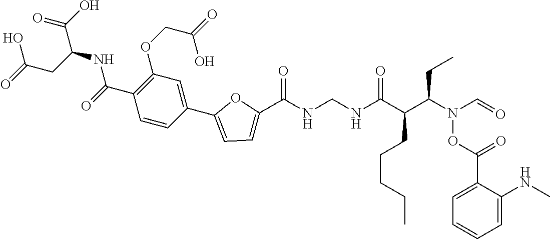 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccccc1NC] | (S)-dibenzyl 2-(2- (2-(benzyloxy)-2- oxoethoxy)-4-(5- ((((R)-2-((R)-1- (N-((2- (methylamino) benzoyl)oxy) formamido) propyl) heptanamido) methyl) carbamoyl) furan-2- yl)benzamido) succinate |
| 24 | (S)-2-(2- (carboxymethoxy)-4- (5-((((R)-2-((R)-1-(N- ((2- ethylbutanoyl)oxy) formamido)propyl) heptanamido)methyl) carbamoyl)furan-2- yl)benzamido) succinic acid | 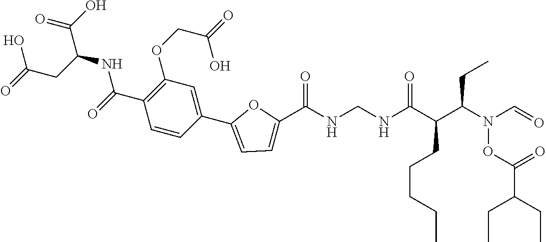 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)C(CC)CC] | (S)-dibenzyl 2-(2- (2-(benzyloxy)-2- oxoethoxy)-4-(5- ((((R)-2-((R)- 1-(N- ((2-ethylbutanoyl) oxy)formamido) propyl) heptanamido) methyl) carbamoyl) furan-2- yl)benzamido) succinate |
| 25 | (S)-2-(2- (carboxymethoxy)-4- (5-((((R)-2-((R)-1-(N- ((3,5- dimethylisoxazole-4- carbonyl)oxy) formamido)propyl) heptanamido)methyl) carbamoyl)furan-2- yl)benzamido) succinic acid | 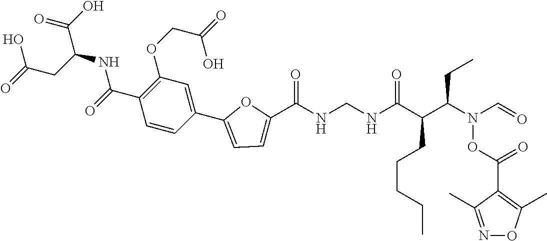 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1c(C)noc1C] | (S)-dibenzyl 2-(2- (2-(benzyloxy)-2- oxoethoxy)-4-(5- ((((R)-2-((R)-1- (N-((3,5- dimethyl- isoxazole- 4-carbonyl)oxy) formamido) propyl) heptanamido) methyl) carbamoyl)furan- 2-yl)benzamido) succinate |
| 26 | (S)-2-(2- (carboxymethoxy)-4- (5-((((R)-2-((R)-1-(N- ((2,4- dimethylnicotinoyl) oxy)formamido) propyl)heptanamido) methyl)carbamoyl) furan-2- yl)benzamido) succinic acid | 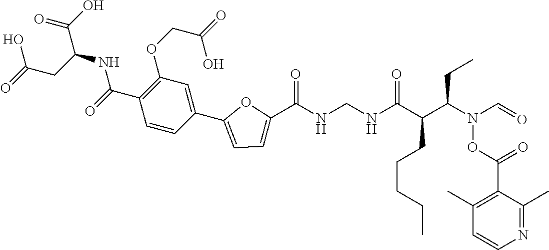 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1c(C)ccnc1C] | (S)-dibenzyl 2-(2- (2-(benzyloxy)-2- oxoethoxy)-4-(5- ((((R)-2-((R)-1- (N-((2,4- dimethyl- nicotinoyl) oxy)formamido) propyl) heptanamido) methyl) carbamoyl) furan-2- yl)benzamido) succinate |
| 27 | (S)-2-(2- (carboxymethoxy)-4- (5-((((R)-2-((R)-1-(N- (2,2- diphenylacetoxy) formamido)propyl) heptanamido)methyl) carbamoyl)furan-2- yl)benzamido) succinic acid | 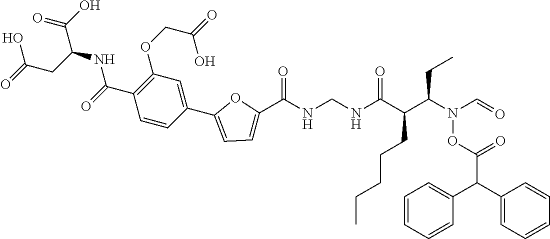 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)C(c1ccccc1)c1ccccc1] | (S)-dibenzyl 2-(2- (2-(benzyloxy)-2- oxoethoxy)-4-(5- ((((R)-2-((R)-1- (N-(2,2- diphenylacetoxy) formamido) propyl) heptanamido) methyl) carbamoyl) furan-2-yl) benzamido) succinate |
| 28 | (S)-2-(2- (carboxymethoxy)-4- (5-((((R)-2-((R)-1- (N-((2- isopropylbenzoyl) oxy) formamido)propyl) heptanamido)methyl) carbamoyl)furan-2- yl)benzamido) succinic acid | 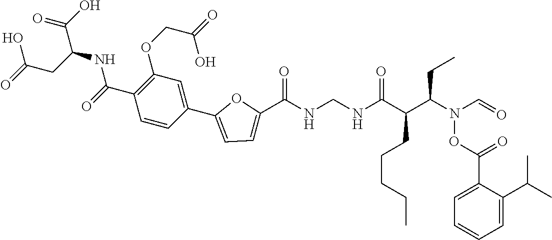 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccccc1C(C)C] | (S)-dibenzyl 2-(2- (2-(benzyloxy)-2- oxoethoxy)-4-(5- ((((R)-2-((R)-1- (N-((2- isopropylbenzoyl) oxy)formamido) propyl) heptanamido) methyl) carbamoyl) furan-2- yl)benzamido) succinate |
| 29 | (S)-2-(2- (carboxymethoxy)-4- (5-(((4R,5R)-5- ethyl-6-formyl- 10,10-dimethyl- 3,8-dioxo-4-pentyl-7- oxa-2,6,9- triazaundecyl) carbamoyl) furan-2-yl) benzamido)succinic acid | 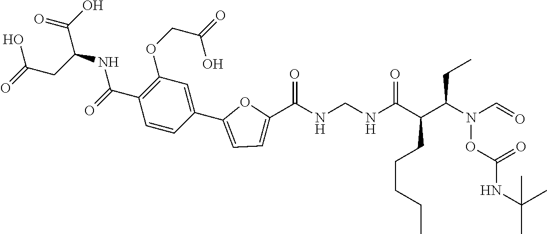 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)NC(C)(C)C] | (S)-dibenzyl 2-(2-(2- (benzyloxy)-2- oxoethoxy)-4- (5-(((4R,5R)- 5-ethyl-6- formyl-10,10- dimethyl-3,8- dioxo-4-pentyl- 7-oxa-2,6,9- triazaundecyl) carbamoyl) furan-2-yl) benzamido) succinate |
| 30 | (S)-2-(2- (carboxymethoxy)-4- (5-(((4R,5R)-5-ethyl- 6-formyl- 10-methyl-3,8- dioxo-4-pentyl-7-oxa- 2,6,9-triazaundecyl) carbamoyl)furan- 2-yl)benzamido) succinic acid | 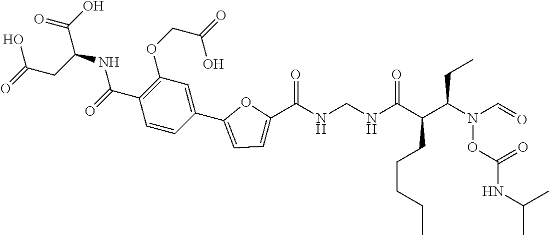 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)NC(C)C] | (S)-dibenzyl 2-(2- (2-(benzyloxy)-2- oxoethoxy)-4-(5- (((4R,5R)- 5-ethyl-6- formyl-10-methyl- 3,8-dioxo-4- pentyl-7-oxa- 2,6,9- triazaundecyl) carbamoyl)furan- 2-yl)benzamido) succinate |
| 31 | (3-(5-((((R)-2-((R)-1- (N-((2- ((dimethylcarbamoyl) oxy)benzoyl)oxy) formamido)propyl) heptanamido)methyl) carbamoyl)furan-2- yl)-5-ethoxyphenyl) phosphonic acid | 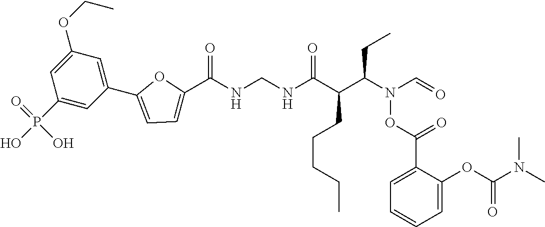 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccccc1OC(=O)N(C)C] | 2-((4R,5R)-10-(5- (3-(bis(benzyloxy) phosphoryl)-5- ethoxyphenyl) furan-2-yl)- 4-ethyl-3-formyl- 6,10-dioxo- 5-pentyl-2-oxa- 3,7,9-triazadecan- 1-oyl)phenyl dimethylcarbamate |
| 32 | (S)-2-(2- (carboxymethoxy)-4- (5-((((R)-2-((R)- 1-(N-((2- ((dimethylcarbamoyl) oxy)benzoyl) oxy)formamido) propyl) heptanamido)methyl) carbamoyl)furan-2- yl)benzamido) succinic acid | 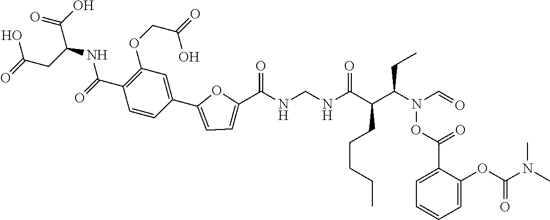 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccccc1OC(=O)N(C)C] | (S)-dibenzyl 2-(2- (2-(benzyloxy)-2- oxoethoxy)-4-(5- ((((R)- 2-((R)-1-(N-((2- ((dimethyl- carbamoyl)oxy) benzoyl)oxy) formamido) propyl) heptanamido) methyl) carbamoyl) furan-2- |
| | | | yl)benzamido) |
| | | | succinate |
| 33 | (S)-2-(4-(5-((((R)-2- ((R)-1-(N-((1- naphthoyl)oxy) formamido) propyl)heptanamido) methyl)carbamoyl) furan-2-yl)-2- (carboxymethoxy) benzamido)succinic acid | 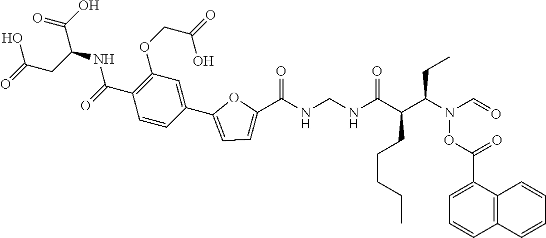 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1cccc2ccccc12] | (S)-dibenzyl 2- (4-(5-((((R)- 2-((R)-1-(N-((1- naphthoyl)oxy) formamido) propyl) heptanamido) methyl) carbamoyl)furan- 2-yl)-2-(2- (benzyloxy)-2- oxoethoxy) benzamido) succinate |
| 34 | (S)-2-(4-(5-((((R)-2- ((R)-1-(N-((2- naphthoyl)oxy) formamido) propyl)heptanamido) methyl)carbamoyl) furan-2-yl)-2- (carboxymethoxy) benzamido) succinic acid | 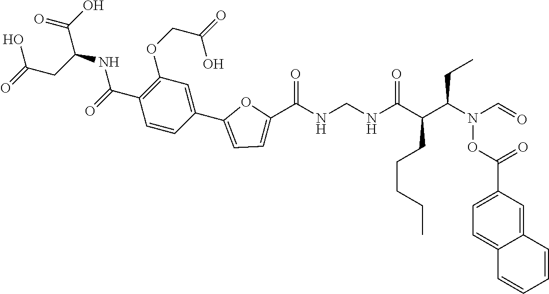 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccc2ccccc2c1] | (S)-dibenzyl 2-(4- (5-((((R)-2-((R)- 1-(N-((2- naphthoyl)oxy) formamido) propyl) heptanamido) methyl) carbamoyl)furan- 2-yl)-2-(2- (benzyloxy)-2- oxoethoxy) benzamido) succinate |
| 35 | (S)-2-(2- (carboxymethoxy)-4- (5-((((R)- 2-((R)-1-(N-((4- (morpholinomethyl) benzoyl)oxy) formamido)propyl) heptanamido)methyl) carbamoyl)furan-2- yl)benzamido) succinic acid | 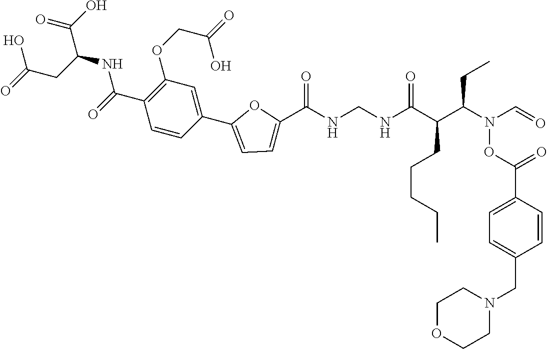 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccc(CN2CCOCC2)cc1] | (S)-dibenzyl 2-(2- (2-(benzyloxy)-2- oxoethoxy)-4-(5- ((((R)-2-((R)-1- (N-((4- (morpholino- methyl) benzoyl)oxy) formamido) propyl) heptanamido) methyl) carbamoyl) furan-2- yl)benzamido) succinate |
| 36 | (S)-2-(2- (carboxymethoxy)-4- (5-(((4R,5R)-5-ethyl- 6-formyl-10-methyl- 3,8-dioxo-4-pentyl- 10-phenyl-7-oxa- 2,6,9- triazaundecyl) carbamoyl)furan-2- yl)benzamido) succinic acid | 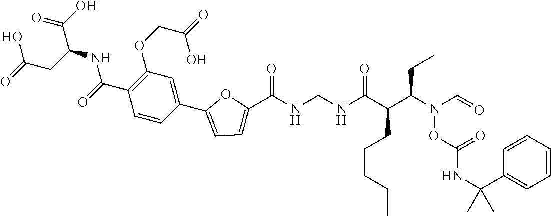 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)NC(C)(C)c1ccccc1] | (S)-dibenzyl 2-(2- (2-(benzyloxy)-2- oxoethoxy)-4-(5- (((4R,5R)-5-ethyl- 6-formyl-10- methyl-3,8-dioxo- 4-pentyl-10- phenyl-7-oxa- 2,6,9- triazaundecyl) carbamoyl)furan- 2-yl)benzamido) succinate |
| 37 | (2S)-2-(2- (carboxymethoxy)-4- (5-((((2R)-2-((1R)-1- (N-(((((S)-1-methoxy- 1-oxopropan-2- yl)amino)(phenoxy) phosphoryl)oxy) formamido)propyl) heptanamido)methyl) carbamoyl) furan-2- yl)benzamido) succinic acid, 3Ammonia salt | 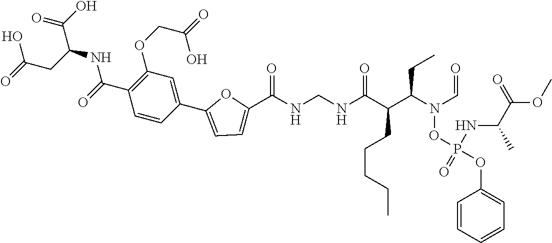 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OP(=O)(N[C@@H](C)C(=O)OC)Oc1ccccc1] | (2S)-dibenzyl 2-(2-(2- (benzyloxy)-2- oxoethoxy)-4- (5-((((2R)-2- ((1R)-1- (N-(((((S)-1- methoxy-1- oxopropan-2- yl)amino) (phenoxy) phosphoryl)oxy) formamido) propyl) heptanamido) methyl) |
| | | | carbamoyl)furan- |
| | | | 2-yl)benzamido) |
| | | | succinate |
| 38 | (S)-2-(2- (carboxymethoxy)-4- (5-((((R)-2-((R)-1-(N- ((2-fluoro-6- methylbenzoyl)oxy) formamido)propyl) heptanamido)methyl) carbamoyl)furan-2- yl)benzamido) succinic acid | 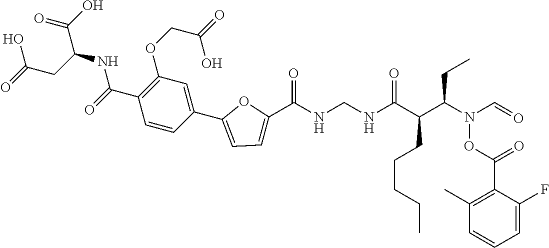 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1c(C)cccc1F] | (S)-dibenzyl 2-(2- (2-(benzyloxy)-2- oxoethoxy)-4-(5- ((((R)-2-((R)-1- (N-((2-fluoro-6- methylbenzoyl) oxy) formamido) propyl) heptanamido) methyl) carbamoyl) furan-2- yl)benzamido) succinate |
| 39 | (S)-2-(2- (carboxymethoxy)-4- (5-((((R)-2-((R)-1-(N- ((4-methoxy-2- methylbenzoyl)oxy) formamido)propyl) heptanamido)methyl) carbamoyl)furan-2- yl)benzamido) succinic acid, 3 Ammonia salt | 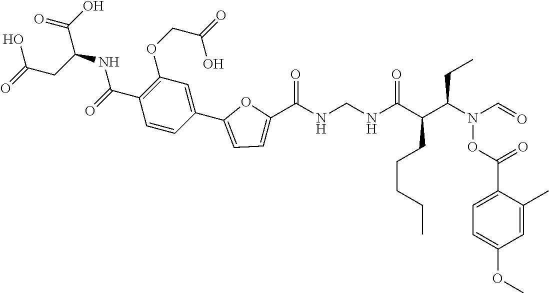 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccc(OC)cc1C] | (S)-dibenzyl 2-(2- (2-(benzyloxy)-2- oxoethoxy)-4-(5- ((((R)-2-((R)-1- (N-((4-methoxy- 2-methyl- benzoyl)oxy) formamido) propyl) heptanamido) methyl) carbamoyl) furan-2- yl)benzamido) succinate |
| 40 | (S)-2-(2- (carboxymethoxy)- 4-(5-((((R)-2-((R)-1- (N-((3- methylisonicotinoyl) oxy)formamido) propyl) heptanamido)methyl) carbamoyl)furan-2- yl)benzamido)succinic acid, 3 Ammonia salt | 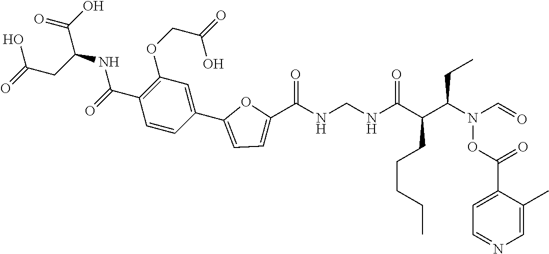 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccncc1C] | (S)-dibenzyl 2-(2- (2-(benzyloxy)-2- oxoethoxy)-4-(5- ((((R)-2-((R)-1- (N-((3- methyl- isonicotinoyl) oxy)formamido) propyl) heptanamido) methyl) carbamoyl) furan-2- yl)benzamido) succinate |
| 41 | (S)-2-(2- (carboxymethoxy)-4- (5-((((R)-2-((R)-1-(N- ((2-methyl-4- (morpholinomethyl) benzoyl)oxy) formamido) propyl)heptanamido) methyl)carbamoyl) furan-2- yl)benzamido) succinic acid | 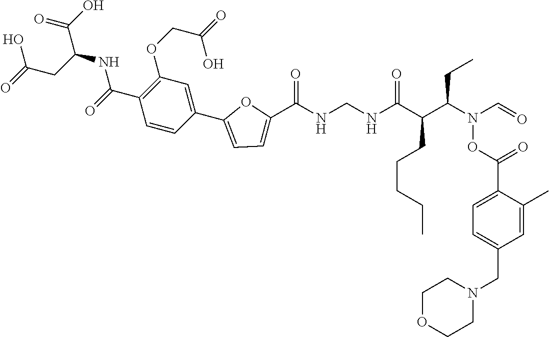 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccc(CN2CCOCC2)cc1C] | (S)-dibenzyl 2-(2- (2-(benzyloxy)-2- oxoethoxy)-4-(5- ((((R)-2-((R)-1- (N-((2-methyl-4- (morpholino- methyl) benzoyl)oxy) formamido) propyl) heptanamido) methyl) carbamoyl) furan- 2-yl)benzamido) succinate |
| 42 | (S)-2-(2- (carboxymethoxy)-4- (5-((((R)-2-((R)-1-(N- ((3,4- dimethoxybenzoyl) oxy)formamido) propyl) heptanamido)methyl) carbamoyl)furan-2- yl)benzamido) succinic acid | 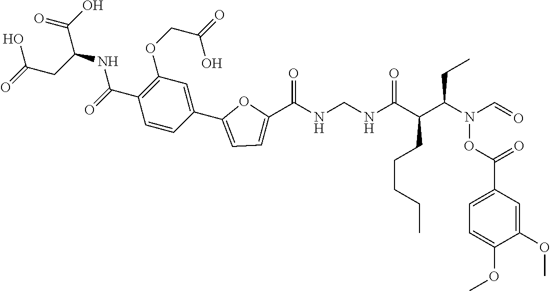 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccc(OC)c(OC)c1] | (S)-dibenzyl 2-(2- (2-(benzyloxy)-2- oxoethoxy)-4-(5- ((((R)-2-((R)-1- (N-((3,4- dimethoxy- benzoyl) oxy)formamido) propyl) heptanamido) methyl) carbamoyl) furan-2- yl)benzamido) succinate |
| 43 | (S)-2-(2- (carboxymethoxy)-4- (5-((((R)-2-((R)-1-(N- ((2- propylpentanoyl)oxy) formamido)propyl) heptanamido)methyl) carbamoyl)furan-2- yl)benzamido) succinic acid | 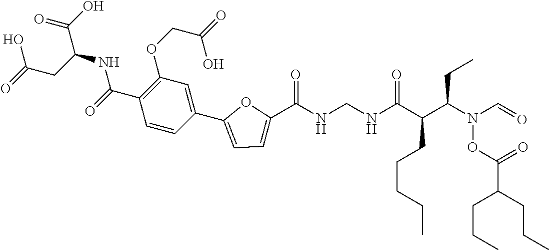 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)C(CCC)CCC] | (S)-dibenzyl 2-(2- (2-(benzyloxy)-2- oxoethoxy)-4-(5- ((((R)-2-((R)-1- (N-((2- propylpentanoyl) oxy)formamido) propyl) heptanamido) methyl) carbamoyl) furan-2-yl) benzamido) succinate |
| 44 | (S)-2-(2- (carboxymethoxy)-4- (5-((((R)-2-((R)-1-(N- ((2,4- dimethoxybenzoyl) oxy)formamido) propyl) heptanamido)methyl) carbamoyl)furan-2- yl)benzamido) succinic acid, 3 Ammonia salt | 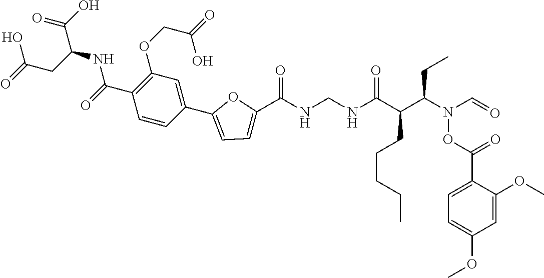 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccc(OC)cc1OC] | (S)-dibenzyl 2-(2- (2-(benzyloxy)-2- oxoethoxy)-4-(5- ((((R)-2-((R)-1- (N-((2,4- dimethoxy- benzoyl) oxy)formamido) propyl) heptanamido) methyl) carbamoyl) furan-2- yl)benzamido) succinate |
| 45 | (3-ethoxy-5-(5-((((R)- 2-((R)-1-(N-((2- ethylbutanoyl)oxy) formamido)propyl) heptanamido)methyl) carbamoyl)furan-2- yl)phenyl)phosphonic acid | 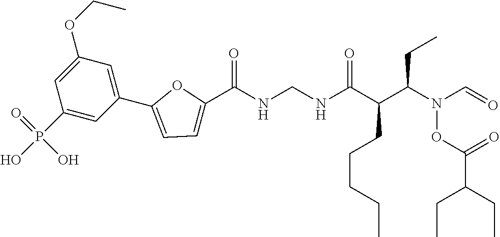 [SMILES: CCCCC[C@@H](C(=O)NCNC(=O)c1ccc(-c2cc(OCC)cc(P(=O)(O)O)c2)o1)[C@@H](CC)N(C=O)OC(=O)C(CC)CC] | dibenzyl (3- ethoxy-5-(5- ((((R)- 2-((R)-1-(N-((2- ethylbutanoyl) oxy) formamido) propyl) heptanamido) methyl) carbamoyl) furan-2- yl)phenyl) phosphonate |
| 46 | (3-ethoxy-5-(5-((((R)- 2-((R)-1-(N-((2-fluoro- 6- methylbenzoyl)oxy) formamido)propyl) heptanamido)methyl) carbamoyl)furan-2- yl)phenyl)phosphonic acid | 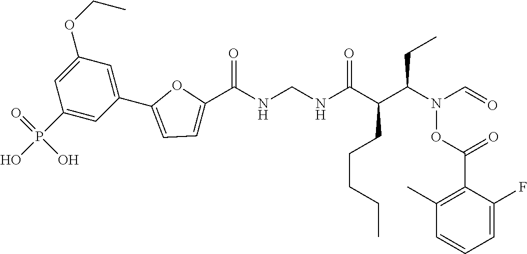 [SMILES: CCCCC[C@@H](C(=O)NCNC(=O)c1ccc(-c2cc(OCC)cc(P(=O)(O)O)c2)o1)[C@@H](CC)N(C=O)OC(=O)c1c(C)cccc1F] | dibenzyl (3- ethoxy-5-(5- ((((R)- 2-((R)-1-(N-((2- fluoro-6- methylbenzoyl) oxy)formamido) propyl) heptanamido) methyl) carbamoyl) furan-2- yl)phenyl) phosphonate |
| 47 | (3-ethoxy-5-(5-((((R)- 2-((R)-1-(N-((4- methoxybenzoyl)oxy) formamido)propyl) heptanamido)methyl) carbamoyl)furan-2- yl)phenyl) phosphonic acid | 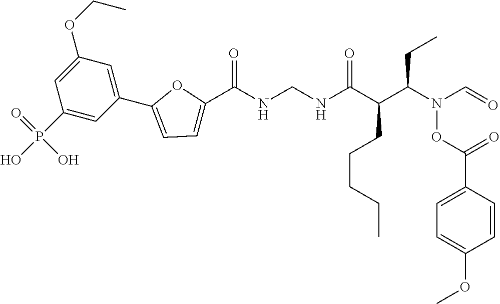 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(OC)cc1] | dibenzyl (3- ethoxy-5-(5- ((((R)- 2-((R)-1-(N-((4- methoxybenzoyl) oxy)formamido) propyl) heptanamido) methyl) carbamoyl) furan-2- yl)phenyl) phosphonate |
| 48 | (S)-2-(2-ethoxy-4-(5- ((((R)-2-((R)-1-(N- ((2-ethylbutanoyl)oxy) formamido)propyl) heptanamido)methyl) carbamoyl)furan-2- yl)benzamido) succinic acid | 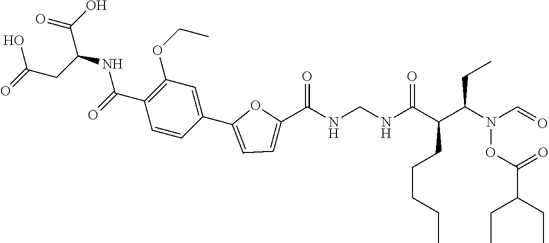 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)C(CC)CC] | (S)-dibenzyl 2-(2-ethoxy-4- (5-((((R)- 2-((R)-1-(N-((2- ethylbutanoyl) oxy) formamido) propyl) heptanamido) methyl) carbamoyl) furan-2- yl)benzamido) succinate |
| 49 | (S)-2-(2-ethoxy-4-(5- ((((R)-2-((R)-1-(N-((2- fluoro-6- methylbenzoyl)oxy) formamido)propyl) heptanamido)methyl) carbamoyl)furan-2- yl)benzamido) succinic acid | 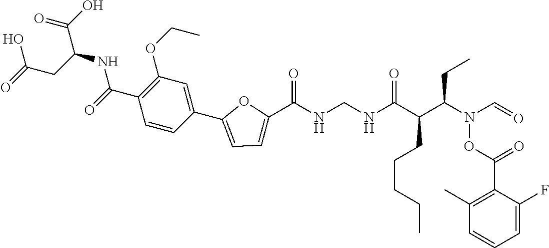 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1c(C)cccc1F] | (S)-dibenzyl 2-(2- ethoxy-4-(5- ((((R)- 2-((R)-1-(N-((2- fluoro-6- methylbenzoyl) oxy) formamido) propyl) heptanamido) methyl) carbamoyl) furan-2- yl)benzamido) succinate |
| 50 | (3-ethoxy-5-(5-((((R)- 2-((R)-1-(N-((4- (pyrrolidin-1- yl)benzoyl)oxy) formamido)propyl) heptanamido)methyl) carbamoyl)furan-2- yl)phenyl)phosphonic acid, 2Ammonia salt | 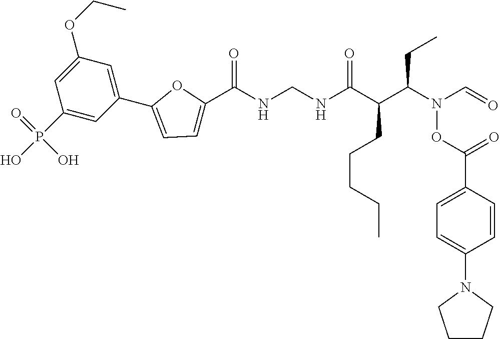 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(N2CCCC2)cc1] | dibenzyl (3- ethoxy-5-(5- ((((R)- 2-((R)-1-(N-((4- (pyrrolidin-1- yl)benzoyl)oxy) formamido) propyl) heptanamido) methyl) carbamoyl)furan- 2-yl)phenyl) phosphonate |
| 51 | (S)-2-(2-ethoxy-4-(5- ((((R)-2-((R)-1-(N- ((4-methoxybenzoyl) oxy)formamido) propyl)heptanamido) methyl)carbamoyl) furan-2-yl) benzamido)succinic acid | 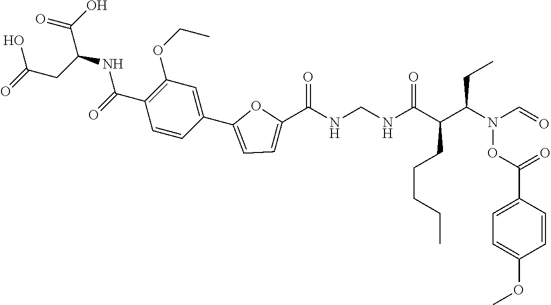 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1] | (S)-dibenzyl 2-(2-ethoxy-4- (5-((((R)- 2-((R)-1-(N-((4- methoxybenzoyl) oxy)formamido) propyl) heptanamido) methyl) carbamoyl) furan-2- yl)benzamido) succinate |
| 52 | (S)-2-(2- (carboxymethoxy)-4- (5-((((R)-2-((R)-1-(N- ((2-isopropyl-4- methoxybenzoyl)oxy) formamido)propyl) heptanamido)methyl) carbamoyl)furan-2- yl)benzamido) succinic acid | 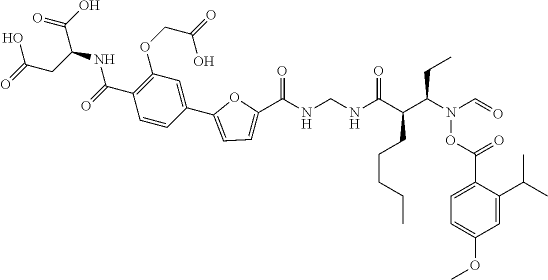 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccc(OC)cc1C(C)C] | (S)-dibenzyl 2-(2- (2-(benzyloxy)-2- oxoethoxy)-4-(5- ((((R)-2-((R)-1- (N-((2- isopropyl-4- methoxybenzoyl) oxy)formamido) propyl) heptanatnido) methyl) carbamoyl) furan-2- yl)benzamido) succinate |
| 53 | (S)-2-(2- (carboxymethoxy)-4- (5-((((R)-2-((R)-1-(N- ((4-fluoro-2- isopropylbenzoyl)oxy) formamido)propyl) heptanamido)methyl) carbamoyl)furan-2- yl)benzamido)succinic acid | 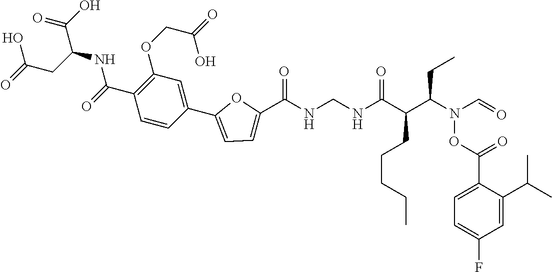 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1ccc(F)cc1C(C)C] | (S)-dibenzyl 2-(2- (2-(benzyloxy)-2- oxoethoxy)-4-(5- ((((R)-2-((R)-1- (N-((4-fluoro-2- isopropylbenzoyl) oxy)formamido) propyl) heptanamido) methyl) carbamoyl) furan-2- yl)benzamido) succinate |
| 54 | (S)-2-(2-ethoxy-4-(5- ((((R)-2-((R)-1-(N- ((4-methoxy-2- methylbenzoyl)oxy) formamido)propyl) heptanamido)methyl) carbamoyl)furan-2- yl)benzamido)succinic acid, 2Ammonia salt | 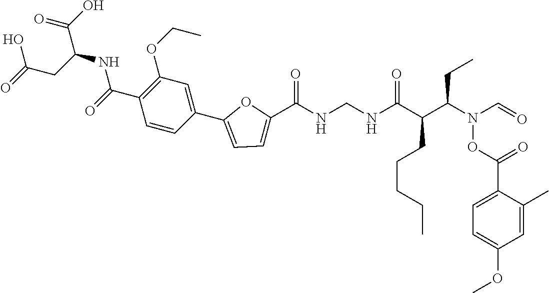 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1C] | (S)-dibenzyl 2-(2- ethoxy-4-(5- ((((R)- 2-((R)-1-(N-((4- methoxy-2- methyl- benzoyl)oxy) formamido) propyl) heptanamido) methyl) carbamoyl) furan-2- yl)benzamido) succinate |
| 55 | (3-ethoxy-5-(5- (((4R,5R)-5-ethyl-6- formyl-10,10- dimethyl-3,8-dioxo-4- pentyl-7-oxa-2,6,9- triazaundecyl) carbamoyl)furan-2- yl)phenyl)phosphonic acid | 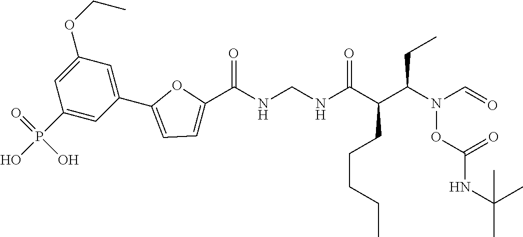 [SMILES: CCCCC[C@@H](C(=O)NCNC(=O)c1ccc(-c2cc(OCC)cc(P(=O)(O)O)c2)o1)[C@@H](CC)N(C=O)OC(=O)NC(C)(C)C] | dibenzyl (3- ethoxy-5-(5- (((4R,5R)-5-ethyl- 6-formyl-10,10- dimethyl-3,8- dioxo-4-pentyl-7- oxa-2,6,9- triazaundecyl) carbamoyl) furan-2- yl)phenyl) phosphonate |
| 56 | (3-ethoxy-5-(5- (((4R,5R)-5-ethyl-6- formyl-10-methyl-3,8- dioxo-4-pentyl-10- phenyl-7-oxa-2,6,9- triazaundecyl) carbamoyl)furan-2- yl)phenyl)phosphonic acid | 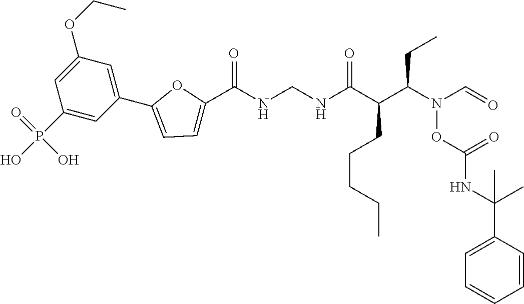 [SMILES: CCCCC[C@@H](C(=O)NCNC(=O)c1ccc(-c2cc(OCC)cc(P(=O)(O)O)c2)o1)[C@@H](CC)N(C=O)OC(=O)NC(C)(C)c1ccccc1] | dibenzyl (3- ethoxy-5-(5- (((4R,5R)-5- ethyl- 6-formyl-10- methyl-3,8-dioxo- 4-pentyl-10- phenyl-7-oxa- 2,6,9- triazaundecyl) carbamoyl)furan- 2-yl)phenyl) phosphonate |
| 57 | (3-ethoxy-5-(5-((((R)- 2-((R)-1-(N-((4- morpholinobenzoyl) oxy)formamido) propyl) heptanamido)methyl) carbamoyl)furan-2- yl)phenyl)phosphonic acid | 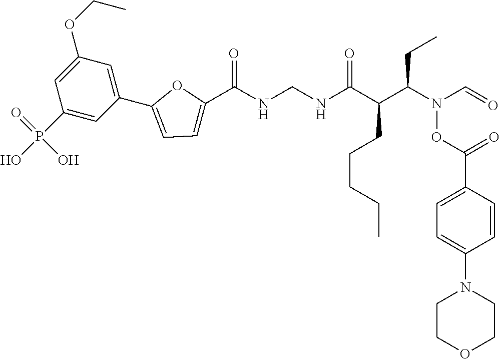 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(N2CCOCC2)cc1] | dibenzyl (3- ethoxy-5-(5- ((((R)- 2-((R)-1-(N-((4- morpholino- benzoyl) oxy)formamido) propyl) heptanamido) methyl) carbamoyl) furan-2- yl)phenyl) phosphonate |
| 58 | (3-(5-((((R)-2-((R)-1- (N-((4-(1H-pyrrol-1- yl)benzoyl)oxy) formamido)propyl) heptanamido)methyl) carbamoyl)furan- 2-yl)-5- ethoxyphenyl) phosphonic acid, 2Ammonia salt | 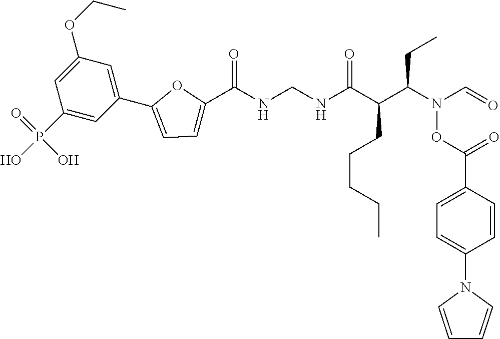 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(-n2cccc2)cc1] | dibenzyl (3-(5- ((((R)-2- ((R)-1-(N- ((4-(1H-pyrrol-1- yl)benzoyl)oxy) formamido) propyl) heptanamido) methyl) carbamoyl) furan-2-yl)-5- ethoxyphenyl) phosphonate |
| 59 | (3-ethoxy-5-(5-((((R)- 2-((R)-1-(N-((4-(2- oxopyrrolidin-1- yl)benzoyl)oxy) formamido)propyl) heptanamido)methyl) carbamoyl)furan-2- yl)phenyl)phosphonic acid, 2Ammonia salt | 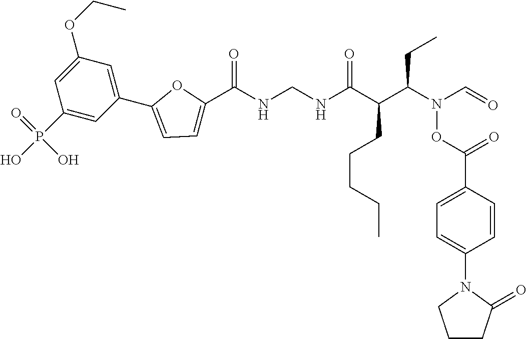 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(N2CCCC2=O)cc1] | dibenzyl (3- ethoxy-5-(5- ((((R)- 2-((R)-1-(N- ((4-(2- oxopyrrolidin-1- yl)benzoyl)oxy) formamido) propyl) heptanamido) methyl) carbamoyl) furan-2-yl) phenyl) phosphonate |
| 60 | (3-ethoxy-5-(5-((((R)- 2-((R)-1-(N-((4- methyl-3,4-dihydro- 2H- benzo[b][1,4]oxazine- 7-carbonyl)oxy) formamido)propyl) heptanamido)methyl) carbamoyl)furan-2- yl)phenyl)phosphonic acid, 2Ammonia salt | 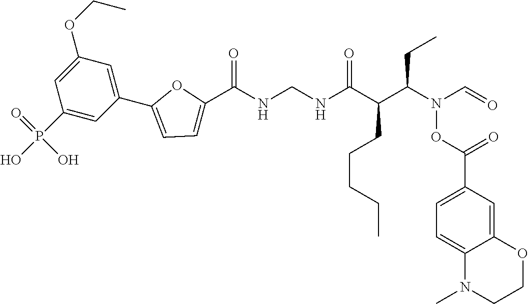 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc2c(c1)OCCN2C] | dibenzyl (3- ethoxy-5-(5- ((((R)- 2-((R)-1-(N-((4- methyl-3,4- dihydro-2H- benzo[b][1,4] oxazine-7- carbonyl)oxy) formamido) propyl) heptanamido) methyl) carbamoyl) furan-2- yl)phenyl) phosphonate |
| 61 | (3-ethoxy-5-(5-((((R)- 2-((R)-1-(N-((2- methyl-6- ((phosphonooxy) methoxy)benzoyl)oxy) formamido)propyl) heptanamido)methyl) carbamoyl)furan-2- yl)phenyl)phosphonic acid, 4Ammonia salt | 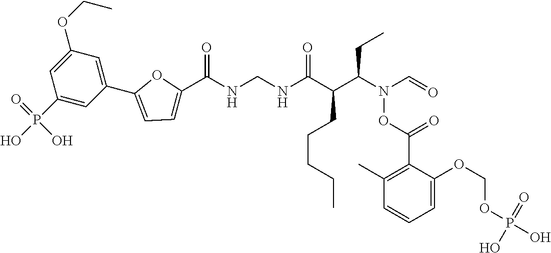 [SMILES: CCCCC[C@@H](C(=O)NCNC(=O)c1ccc(-c2cc(OCC)cc(P(=O)(O)O)c2)o1)[C@@H](CC)N(C=O)OC(=O)c1c(C)cccc1OCOP(=O)(O)O] | (2-((4R,5R)- 10-(5- (3-(bis (benzyloxy) phosphoryl)-5- ethoxyphenyl) furan-2-yl)- 4-ethyl-3- formyl-6,10- dioxo- 5-pentyl-2-oxa- 3,7,9-triazadecan- 1-oyl)-3- methylphenoxy) methyl dihydrogen |
| | | | phosphate |
| 62 | (3-ethoxy-5-(5-((((R)- 2-((R)-1-(N-((3-fluoro- 4-(pyrrolidin-1- yl)benzoyl)oxy) formamido)propyl) heptanamido)methyl) carbamoyl)furan-2- yl)phenyl)phosphonic acid, 2Ammonia salt | 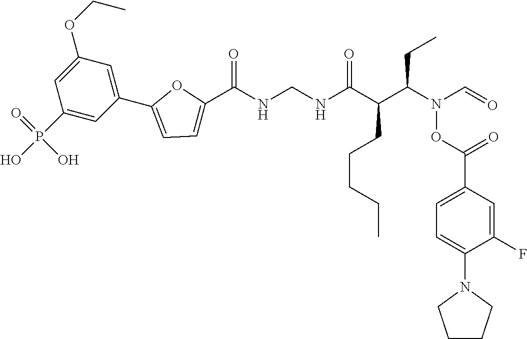 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(N2CCCC2)c(F)c1] | dibenzyl (3- ethoxy-5-(5- ((((R)- 2-((R)-1-(N-((3- fluoro-4- (pyrrolidin-1- yl)benzoyl)oxy) formamido) propyl) heptanamido) methyl) carbamoyl) furan-2- yl)phenyl) phosphonate |
| 63 | (3-ethoxy-5-(5-((((R)- 2-((R)-1-(N-((3- (pyrrolidin-1- yl)benzoyl)oxy) formamido)propyl) heptanamido)methyl) carbamoyl)furan-2- yl)phenyl)phosphonic acid, 2Ammonia salt | 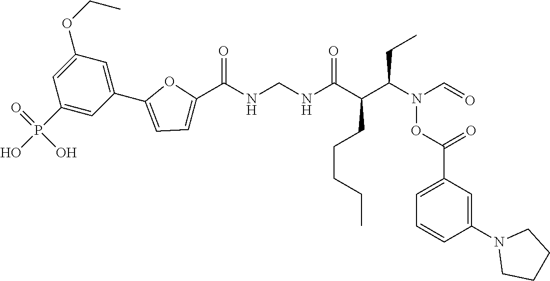 [SMILES: CCCCC[C@@H](C(=O)NCNC(=O)c1ccc(-c2cc(OCC)cc(P(=O)(O)O)c2)o1)[C@@H](CC)N(C=O)OC(=O)c1cccc(N2CCCC2)c1] | dibenzyl (3- ethoxy-5-(5- ((((R)- 2-((R)-1-(N-((3- (pyrrolidin-1- yl)benzoyl)oxy) formamido) propyl) heptanamido) methyl) carbamoyl) furan-2- yl)phenyl) phosphonate |
| 64 | (3-ethoxy-5-(5-((((R)- 2-((R)-1-(N-((2- methyl-4- (morpholinomethyl) benzoyl)oxy) formamido) propyl)heptanamido) methyl)carbamoyl) furan-2- yl)phenyl)phosphonic acid | 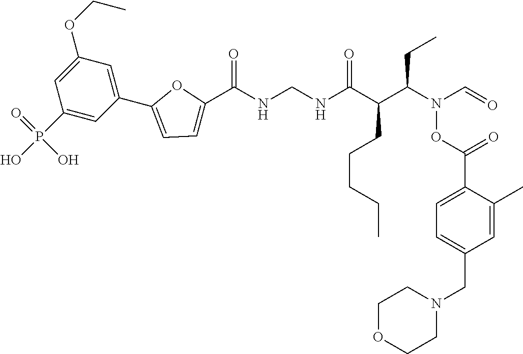 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(CN2CCOCC2)cc1C] | dibenzyl (3- ethoxy-5- (5-((((R)- 2-((R)-1-(N-((2- methyl-4- (morpholino- methyl) benzoyl)oxy) formamido) propyl) heptanamido) methyl) carbamoyl) furan-2-yl) phenyl) phosphonate |
| 65 | (3-ethoxy-5-(5- (((4R,5R)-5-ethyl-6- formyl-10-methyl-3,8- dioxo-4-pentyl-7-oxa- 2,6,9- triazaundecyl) carbamoyl)furan-2- yl)phenyl)phosphonic acid | 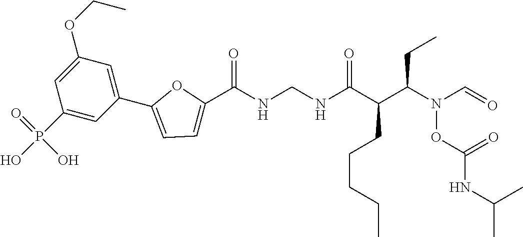 [SMILES: CCCCC[C@@H](C(=O)NCNC(=O)c1ccc(-c2cc(OCC)cc(P(=O)(O)O)c2)o1)[C@@H](CC)N(C=O)OC(=O)NC(C)C] | dibenzyl (3- ethoxy-5-(5- (((4R,5R)-5- ethyl-6-formyl- 10-methyl-3,8- dioxo- 4-pentyl-7-oxa- 2,6,9- triazaundecyl) carbamoyl) furan-2- yl)phenyl) phosphonate |
| 66 | (S)-2-(2-ethoxy-4-(5- ((((R)-2-((R)-1-(N-((2- isopropyl-4- methoxybenzoyl)oxy) formamido)propyl) heptanamido)methyl) carbamoyl)furan-2- yl)benzamido) succinic acid | 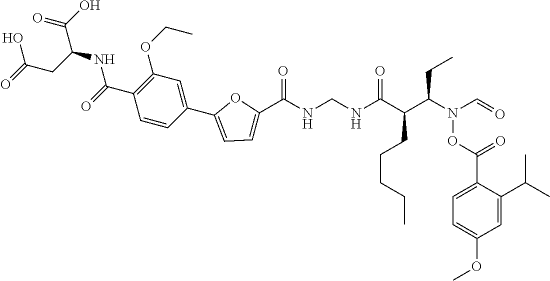 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1C(C)C] | (S)-dibenzyl 2-(2-ethoxy-4- (5-((((R)- 2-((R)-1-(N-((2- isopropyl-4- methoxybenzoyl) oxy)formamido) propyl) heptanatmido) methyl) carbamoyl) furan-2- yl)benzamido) succinate |
| 67 | (S)-2-(2-ethoxy-4-(5- ((((R)-2-((R)-1-(N- ((4-fluoro-2- isopropylbenzoyl) oxy)formamido) propyl)heptanamido) methyl)carbamoyl) furan-2-yl) benzamido)succinic acid | 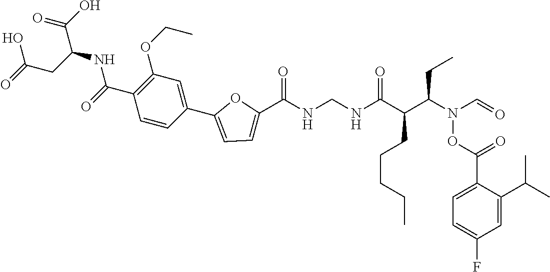 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(F)cc1C(C)C] | (S)-dibenzyl 2-(2-ethoxy-4- (5-((((R)- 2-((R)-1-(N-((4- fluoro-2- isopropyl- benzoyl) oxy)formamido) propyl) heptanamido) methyl) carbamoyl) furan-2- yl)benzamido) succinate |
| 68 | (3-ethoxy-5-(5-((((R)- 2-((R)-1-(N-((4- fluoro-2- isopropylbenzoyl)oxy) formamido)propyl) heptanamido)methyl) carbamoyl)furan-2- yl)phenyl)phosphonic acid | 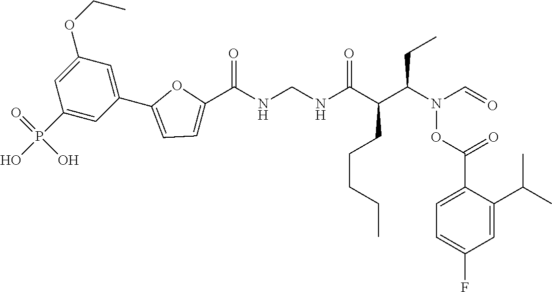 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(F)cc1C(C)C] | dibenzyl (3- ethoxy-5-(5- ((((R)- 2-((R)-1-(N-((4- fluoro-2- isopropylbenzoyl) oxy)formamido) propyl) heptanamido) methyl) carbamoyl) furan-2- yl)phenyl) phosphonate |
| 69 | (3-ethoxy-5-(5-((((R)- 2-((R)-1-(N- (nonanoyloxy) formamido)propyl) heptanamido)methyl) carbamoyl) furan-2- yl)phenyl)phosphonic acid | 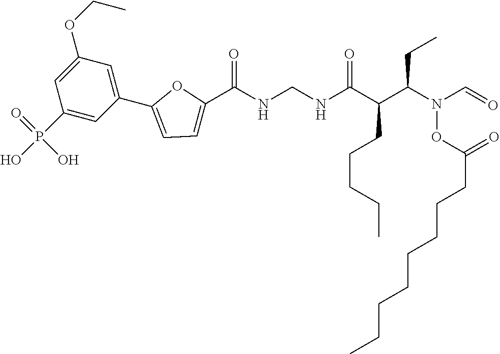 [SMILES: CCCCCCCCC(=O)ON(C=O)[C@H](CC)[C@@H](CCCCC)C(=O)NCNC(=O)c1ccc(-c2cc(OCC)cc(P(=O)(O)O)c2)o1] | dibenzyl (3- ethoxy-5-(5- ((((R)- 2-((R)-1-(N- (nonanoyloxy) formamido) propyl) heptanamido) methyl) carbamoyl) furan-2-yl) phenyl) phosphonate |
| 70 | (S)-2-(2-ethoxy-4-(5- ((((R)-2-((R)-1-(N-((4- (morpholinomethyl) benzoyl)oxy) formamido) propyl)heptanamido) methyl)carbamoyl) furan-2- yl)benzamido) succinic acid | 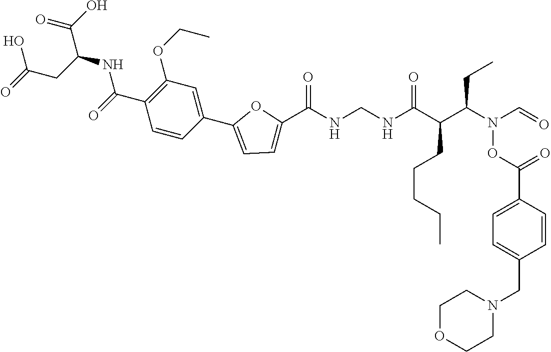 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(CN2CCOCC2)cc1] | (S)-dibenzyl 2-(2- ethoxy-4-(5- ((((R)- 2-((R)-1-(N-((4- (morpholino- methyl) benzoyl)oxy) formamido) propyl) heptanamido) methyl) carbamoyl) furan-2-yl) benzamido) succinate |
| 71 | (S)-2-(2-ethoxy-4-(5- ((((R)-2-((R)-1-(N-((2- methyl-4- (morpholinomethyl) benzoyl)oxy) formamido) propyl)heptanamido) methyl)carbamoyl) furan-2- yl)benzamido) succinic acid | 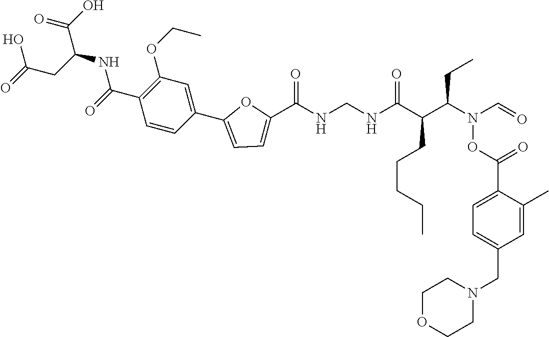 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(CN2CCOCC2)cc1C] | (S)-dibenzyl 2-(2- ethoxy-4-(5- ((((R)- 2-((R)-1-(N-((2- methyl-4- (morpholino- methyl) benzoyl)oxy) formamido) propyl) heptanamido) methyl) carbamoyl) furan-2- yl)benzamido) succinate |
| 72 | (S)-2-(2-ethoxy-4-(5- (((4R,5R)-5-ethyl-6- formyl-10-methyl-3,8- dioxo-4-pentyl-7-oxa- 2,6,9- triazaundecyl) carbamoyl)furan-2- yl)benzamido) succinic acid | 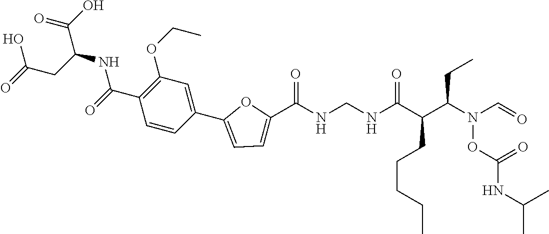 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)NC(C)C] | (S)-dibenzyl 2-(2- ethoxy-4-(5- (((4R,5R)-5- ethyl- 6-formyl-10- methyl-3,8-dioxo- 4-pentyl-7-oxa- 2,6,9- triazaundecyl) carbamoyl) furan-2- yl)benzamido) succinate |
| 73 | (3-ethoxy-5-(5-((((R)- 2-((R)-1-(N-((4- (morpholinomethyl) benzoyl)oxy) formamido) propyl)heptanamido) methyl)carbamoyl) furan-2- yl)phenyl)phosphonic acid | 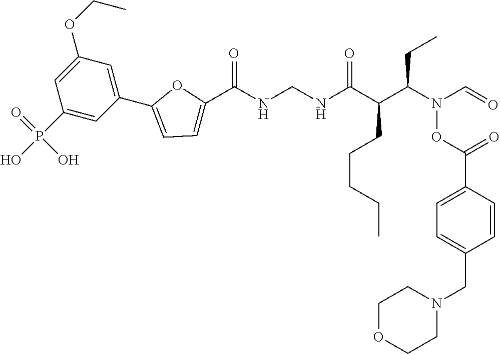 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(CN2CCOCC2)cc1] | dibenzyl (3- ethoxy-5-(5- ((((R)- 2-((R)-1-(N-((4- (morpholino- methyl) benzoyl)oxy) formamido)propyl) heptanamido) methyl) carbamoyl) furan-2-yl) phenyl) phosphonate |
| 74 | (S)-2-(2-ethoxy-4-(5- (((4R,5R)-5-ethyl-6- formyl-10,10- dimethyl-3,8-dioxo- 4-pentyl-7-oxa- 2,6,9-triazaundecyl) carbamoyl)furan-2- yl)benzamido) succinic acid | 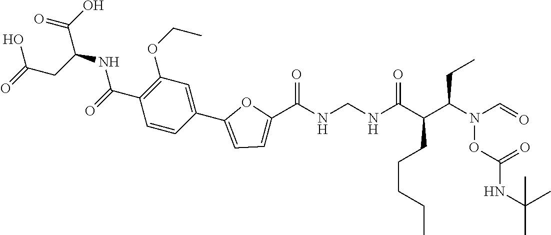 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)NC(C)(C)C] | (S)-dibenzyl 2-(2- ethoxy-4-(5- (((4R,5R)-5-ethyl- 6-formyl-10,10- dimethyl-3,8- dioxo-4-pentyl-7- oxa-2,6,9- triazaundecyl) carbamoyl)furan- 2-yl)benzamido) succinate |
| 75 | (S)-2-(2-ethoxy-4-(5- (((4R,5R)-5-ethyl-6- formyl-10-methyl-3,8- dioxo-4-pentyl-10- phenyl-7-oxa-2,6,9- triazaundecyl) carbamoyl)furan-2- yl)benzamido) succinic acid | 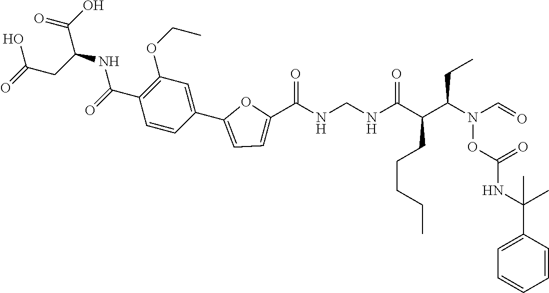 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)NC(C)(C)c1ccccc1] | (S)-dibenzyl 2-(2- ethoxy-4-(5- (((4R,5R)-5-ethyl- 6-formyl-10- methyl-3,8-dioxo- 4-pentyl-10- phenyl-7-oxa- 2,6,9- triazaundecyl) carbamoyl) furan-2- yl)benzamido) succinate |
| 76 | (S)-2-(2-ethoxy-4-(5- ((((R)-2-((R)-1-(N- ((3-(pyrrolidin-1- yl)benzoyl)oxy) formamido)propyl) heptanamido)methyl) carbamoyl)furan-2- yl)benzamido) succinic acid, 2Ammonia salt | 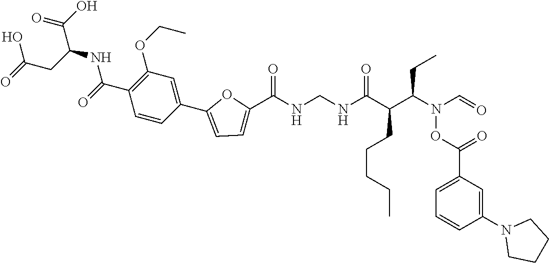 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1cccc(N2CCCC2)c1] | (S)-dibenzyl 2-(2- ethoxy-4-(5-((((R)- 2-((R)-1-(N-((3- (pyrrolidin-1- yl)benzoyl)oxy) formamido)propyl) heptanamido) methyl) carbamoyl)furan-2- yl)benzamido) succinate |
| 77 | (S)-2-(2-ethoxy-4-(5- ((((R)-2-((R)-1-(N-((3- fluoro-4-(pyrrolidin-1- yl)benzoyl)oxy) formamido)propyl) heptanamido)methyl) carbamoyl)furan-2- yl)benzamido)succinic acid, 2Ammonia salt | 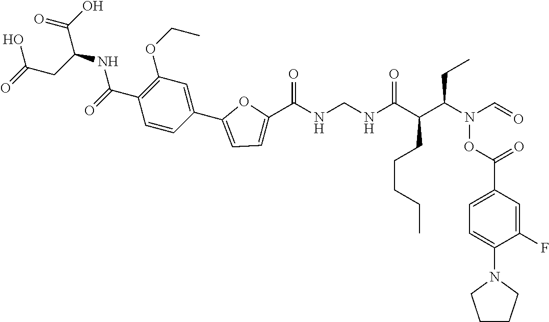 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(N2CCCC2)c(F)c1] | (S)-dibenzyl 2-(2- ethoxy-4-(5-((((R)- 2-((R)-1-(N-((3- fluoro-4- (pyrrolidin-1- yl)benzoyl)oxy) formamido)propyl) heptanamido (methyl) carbamoyl) furan-2- yl)benzamido) succinate |
| 78 | (S)-2-(2-ethoxy-4-(5- ((((R)-2-((R)-1-(N- ((4-(2-oxopyrrolidin- 1-yl)benzoyl)oxy) formamido)propyl) heptanamido)methyl) carbamoyl)furan-2- yl)benzamido) succinic acid, 2Ammonia salt | 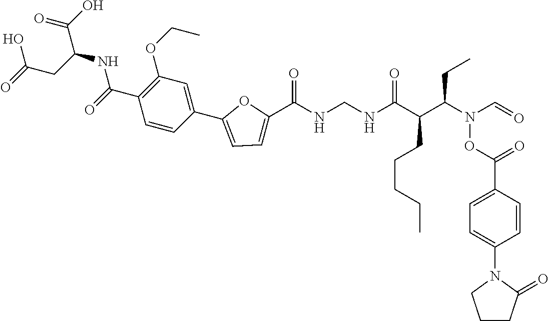 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(N2CCCC2=O)cc1] | (S)-dibenzyl 2-(2- ethoxy-4-(5-((((R)- 2-((R)-1-(N-((4-(2- oxopyrrolidin-1- yl)benzoyl)oxy) formamido)propyl) heptanamido (methyl) carbamoyl) furan-2- yl)benzamido) succinate |
| 79 | (S)-2-(4-(5-((((R)-2- ((R)-1-(N-((4-(1H- pyrrol-1- yl)benzoyl)oxy) formamido)propyl) heptanamido)methyl) carbamoyl)furan-2- yl)-2- ethoxybenzamido) succinic acid, 2Ammonia salt | 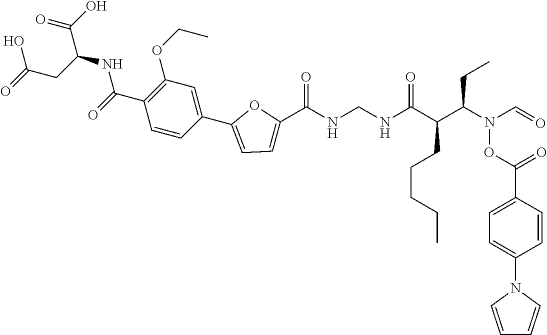 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(-n2cccc2)cc1] | (S)-dibenzyl 2-(4- (5-((((R)-2-((R)-1- (N-((4-(1H-pyrrol- 1-yl)benzoyl)oxy) formamido)propyl) heptanamido) methyl) carbamoyl) furan-2-yl)-2- ethoxybenzamido) succinate |
| 80 | (S)-2-(2-ethoxy-4-(5- ((((R)-2-((R)-1-(N- ((phosphonooxy) methoxy)formamido) propyl)heplanamido) methyl) carbamoyl)furan-2- yl)benzamido) succinic acid, Ammonia salt | 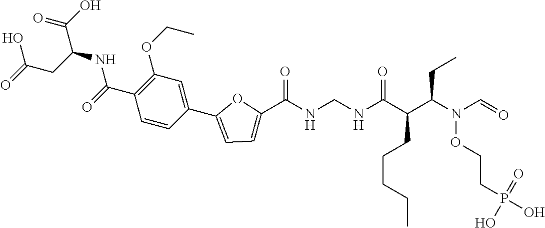 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OCCP(=O)(O)O] | (2S)-dibenzyl 2-(4- (5-((((2R)-2-((1R)- 1-(N- ((((benzyloxy) (hydroxy) phosphoryl) oxy)methoxy) formamido)propyl) heptanamido) methyl)carbamoyl) furan-2-yl)-2- ethoxybenzamido) succinate |
| 81 | (3-(5-((((R)-2-((R)-1- (N-((2,3- dihydrobenzo[b][1,4] dioxine-6- carbonyl)oxy) formamido)propyl) heptanamido)methyl) carbamoyl) furan-2-yl)-5- ethoxyphenyl) phosphonic acid | 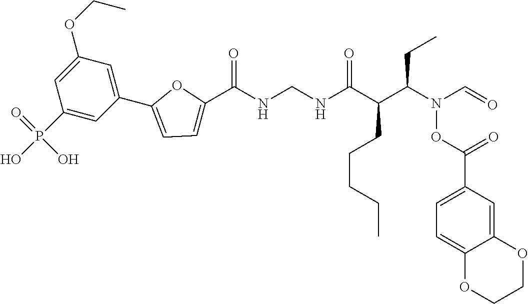 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc2c(c1)OCCO2] | dibenzyl (3-(5- ((((R)-2-((R)-1- (N-((2,3- dihydrobenzo[b] [1,4]dioxine-6- carbonyl)oxy) formamido)propyl) heptanamido) methyl) carbamoyl)furan- 2-yl)-5- ethoxyphenyl) phosphonate |
| 82 | (3-ethoxy-5-(5- (((4R,5R)-5-ethyl-6- formyl-10,10- dimethyl-3,8-dioxo-4- pentyl-7,9-dioxa-2,6- diazaundecyl) carbamoyl)furan-2- yl)phenyl) phosphonic acid | 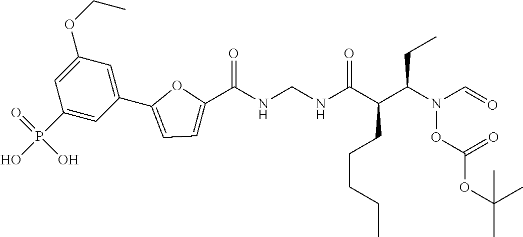 [SMILES: CCCCC[C@@H](C(=O)NCNC(=O)c1ccc(-c2cc(OCC)cc(P(=O)(O)O)c2)o1)[C@@H](CC)N(C=O)OC(=O)OC(C)(C)C] | dibenzyl (3- ethoxy-5-(5- (((4R,5R)-5-ethyl- 6-formyl-10,10- dimethyl-3,8- dioxo-4-pentyl- 7,9-dioxa-2,6- diazaundecyl) carbamoyl) furan-2- yl)phenyl) phosphonate |
| 83 | (S)-2-(4-(5-((((R)-2- ((R)-1-(N-((2,3- dihydrobenzo[b] [1,4]dioxine-6- carbonyl)oxy) formamido)propyl) heptanamido)methyl) carbamoyl)furan-2-yl)- 2-ethoxybenzamido) succinic acid, 2Ammonia salt | 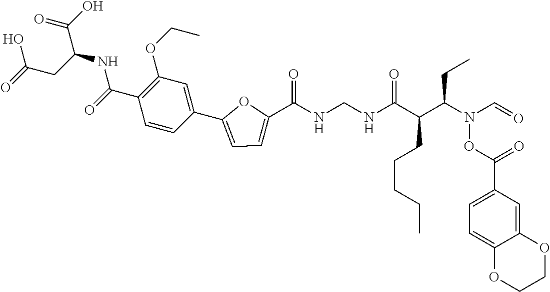 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc2c(c1)OCCO2] | (S)-dibenzyl 2-(4- (5-((((R)-2-((R)-1- (N-((2,3- dihydrobenzo[b] [1,4]dioxine-6- carbonyl)oxy) formamido) propyl) heptanamido) methyl) carbamoyl) furan-2-yl)-2- ethoxybenzamido) succinate |
| 84 | (3-ethoxy-5-(5-((((R)- 2-((R)-1-(N-((4- methyl-3,4-dihydro- 2H- benzo[b][1,4]oxazine- 6-carbonyl)oxy) formamido)propyl) heptanamido)methyl) carbamoyl)furan-2- yl)phenyl)phosphonic acid, 2Ammonia salt | 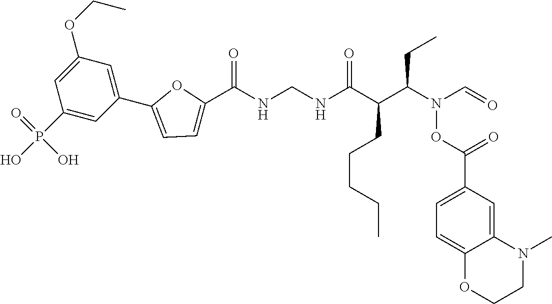 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc2c(c1)N(C)CCO2] | dibenzyl (3- ethoxy-5-(5- ((((R)-2-((R)-1- (N-((4-methyl- 3,4-dihydro-2H- benzo[b][1,4] oxazine-6- carbonyl)oxy) formamido) propyl) heptanamido) methyl)carbamoyl) furan-2-yl)phenyl) phosphonate |
| 85 | (S)-2-(2-ethoxy-4-(5- ((((R)-2-((R)-1-(N-((4- methyl-3,4-dihydro- 2H- benzo[b][1,4]oxazine- 6-carbonyl)oxy) formamido)propyl) heptanamido)methyl) carbamoyl)furan-2- yl)benzamido) succinic acid, 2Ammonia salt | 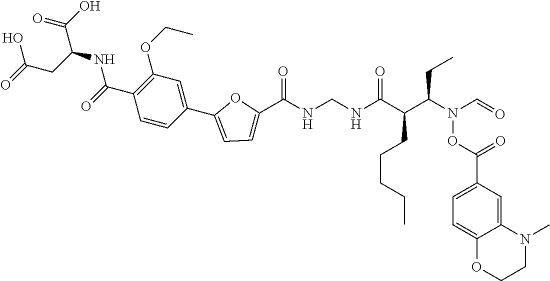 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc2c(c1)N(C)CCO2] | (S)-dibenzyl 2-(2- ethoxy-4-(5-((((R)- 2-((R)-1-(N-((4- methyl-3,4- dihydro-2H- benzo(b)[1,4] oxazine-6- carbonyl)oxy) formamido)propyl) heptanamido) methyl) carbamoyl)furan- 2-yl)benzamido) succinate |
| 86 | (S)-2-(2-ethoxy-4-(5- (((4R,5R)-5-ethyl-6- formyl-10,10- dimethyl-3,8-dioxo-4- pentyl-7,9-dioxa-2,6- diazaundecyl) carbamoyl)furan-2- yl)benzamido) succinic acid | 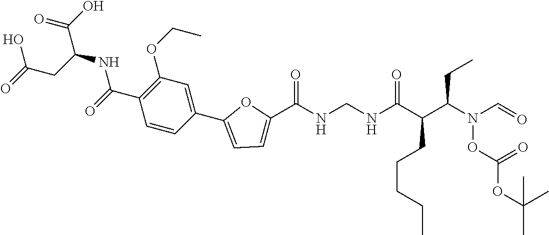 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)OC(C)(C)C] | (S)-dibenzyl 2-(2- ethoxy-4-(5- (((4R,5R)-5-ethyl- 6-formyl-10,10- dimethyl-3,8- dioxo-4-pentyl- 7,9-dioxa-2,6- diazaundecyl) carbamoyl)furan-2- yl)benzamido) succinate |
| 87 | (2S)-2-(2- (carboxymethoxy)-4- (5-((((R)-2-((R)-1-(N- ((2-methoxy-6- methylbenzoyl)oxy) formamido)propyl) heptanamido)methyl) carbamoyl) tetrahydrofuran-2- yl)benzamido)succinic acid | 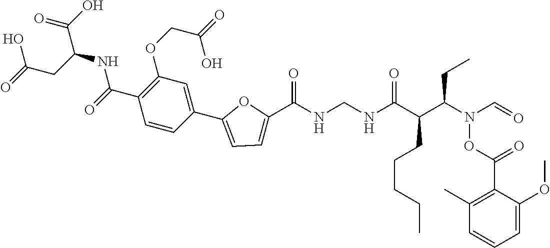 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)c1c(C)cccc1OC] | (S)-dibenzyl 2-(2- (2-(benzyloxy)-2- oxoethoxy)-4-(5- ((((R)-2-((R)-1-(N- ((2-methoxy-6- methylbenzoyl) oxy)formamido) propyl) heptanamido) methyl) carbamoyl) furan-2- yl)benzamido) succinate |
| 88 | (3-ethoxy-5-(5-((((R)- 2-((R)-1-(N-((4- methoxy-2- methylbenzoyl)oxy) formamido)propyl) heptanamido) methyl)carbamoyl) furan-2- yl)phenyl)phosphonic acid, 2Ammonia salt | 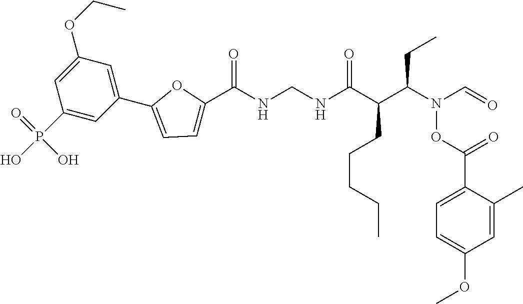 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(OC)cc1C] | dibenzyl (3- ethoxy-5-(5-((((R)- 2-((R)-1-(N-((4- methoxy-2- methylbenzoyl) oxy)formamido) propyl) heptanamido) methyl) carbamoyl) furan-2- yl)phenyl) phosphonate |
| 89 | (3-ethoxy-5-(5-((((R)- 2-((R)-1-(N-((2- isopropyl-4- methoxybenzoyl)oxy) formamido)propyl) heptanamido)methyl) carbamoyl)furan-2- yl)phenyl)phosphonic acid | 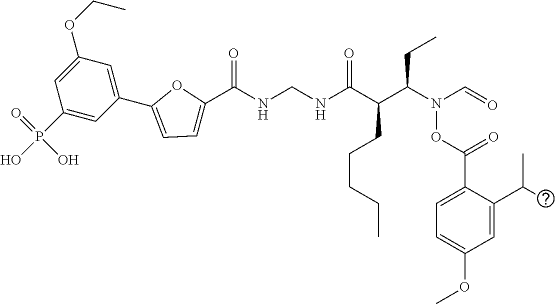 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(OC)cc1C(C)C] | dibenzyl (3- ethoxy-5-(5-((((R)- 2-((R)-1-(N-((2- isopropyl-4- methoxybenzoyl) oxy)formamido) propyl) heptanamido) methyl) carbamoyl) furan-2-yl)phenyl) phosphonate |
| 90 | (S)-2-(2- (carboxymethoxy)-4- (5-((((R)-2-((R)-1-(N- ((phenylcarbamoyl) oxy)formamido) propyl)heptanamido) methyl)carbamoyl) furan-2- yl)benzamido) succinic acid | 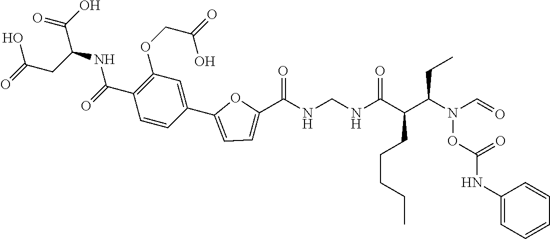 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)Nc1ccccc1] | (S)-dibenzyl 2-(2- (2-(benzyloxy)-2- oxoethoxy)-4-(5- ((((R)-2-((R)-1-(N- ((phenylcarbamoyl) oxy)formamido) propyl) heptanamido) methyl)carbamoyl) furan-2- yl)benzamido) succinate |
| 91 | (S)-2-(4-(5-((((R)-2- ((R)-1-(N-((3,4- dimethoxybenzoyl) oxy)formamido) propyl)heptanamido) methyl) carbamoyl) furan-2-yl)-2- ethoxybenzamido) succinic acid | 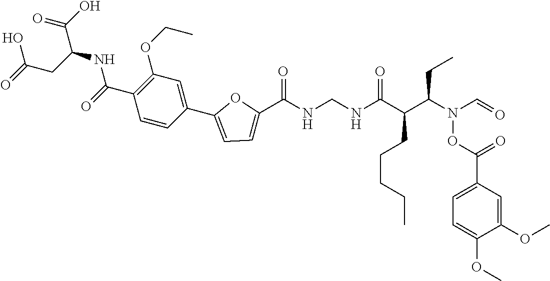 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)c(OC)c1] | (S)-dibenzyl 2-(4- (5-((((R)-2-((R)-1- (N-((3,4- dimethoxybenzoyl) oxy)formamido) propyl) heptanamido) methyl)carbamoyl) furan-2-yl)-2- ethoxybenzamido) succinate |
| 92 | (3-ethoxy-5-(5-((((R)- 2-((R)-1-(N- ((phenylcarbamoyl) oxy)formamido) propyl)heptanamido) methyl)carbamoyl) furan-2- yl)phenyl)phosphonic acid | 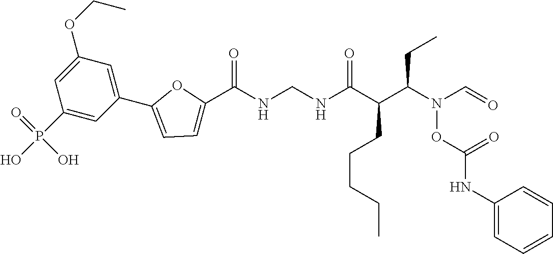 [SMILES: CCCCC[C@@H](C(=O)NCNC(=O)c1ccc(-c2cc(OCC)cc(P(=O)(O)O)c2)o1)[C@@H](CC)N(C=O)OC(=O)Nc1ccccc1] | dibenzyl (3- ethoxy-5-(5-((((R)- 2-((R)-1-(N- ((phenylcarbamoyl) oxy)formamido) propyl) heptanamido) methyl)carbamoyl) furan-2-yl) phenyl) phosphonate |
| 93 | (S)-2-(2-ethoxy-4-(5- ((((R)-2-((R)-1-(N-((2- methylbenzoyl)oxy) formamido)propyl) heptanamido)methyl) carbamoyl)furan-2- yl)benzamido) succinic acid | 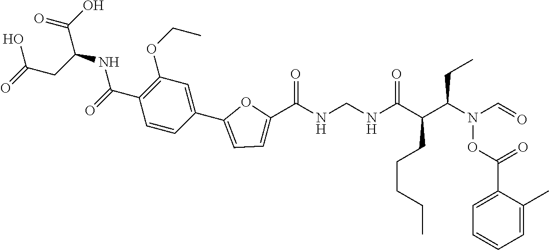 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccccc1C] | (S)-dibenzyl 2-(2- ethoxy-4-(5-((((R)- 2-((R)-1-(N-((2- methylbenzoyl) oxy)formamido) propyl) heptanamido) methyl) carbamoyl) furan-2- yl)benzamido) succinate |
| 94 | (S)-2-(2-ethoxy-4-(5- (((4R,5R)-5-ethyl-6- formyl-11,11- dimethyl-3,10-dioxo- 4-pentyl-7,9-dioxa- 2,6-diazadodecyl) carbamoyl)furan-2- yl)benzamido) succinic acid | 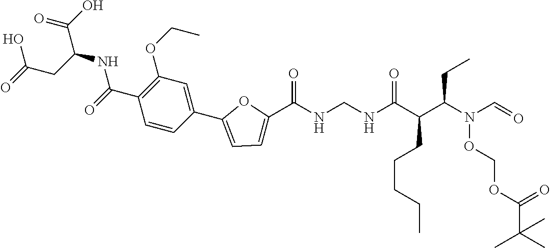 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OCOC(=O)C(C)(C)C] | (S)-dibenzyl 2-(2- ethoxy-4-(5- (((4R,5R)-5-ethyl- 6-formyl-11,11- dimethyl-3,10- dioxo-4-pentyl- 7,9-dioxa-2,6- diazadodecyl) carbamoyl) furan-2- yl)benzamido) succinate |
| 95 | N-{[2- (carboxymethoxy)-4- (5-{[(4R,5R)-5-ethyl- 6-formyl-9,12,12- trimethyl-3,8,10- trioxo-4-pentyl-7,11- dioxa-2,6,9- triazatridec-1- yl]carbamoyl}furan- 2-yl)phenyl] carbonyl}-L- aspartic acid | 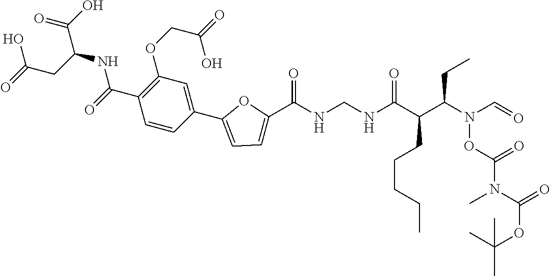 [SMILES: CCCCC[C@@H](C(=O)NCNC(=O)c1ccc(-c2ccc(C(=O)N[C@@H](CC(=O)O)C(=O)O)c(OCC(=O)O)c2)o1)[C@@H](CC)N(C=O)OC(=O)N(C)C(=O)OC(C)(C)C] | dibenzyl N-({2-[2- (benzyloxy)-2- oxoethoxy]-4-(5- {[(4R,5R)-5-ethyl- 6-formyl-9,12,12- trimethyl-3,8,10- trioxo-4-pentyl- 7,11-dioxa-2,6,9- triazatridec-1- yl]carbamoyl} furan-2- yl)phenyl} carbonyl)-L- aspartate |
| 96 | (S)-2-(4-(5-((((R)-2- ((R)-1-(N-((2,4- dimethoxybenzoyl) oxy)formamido) propyl)heptanamido) methyl)carbamoyl) furan-2-yl)-2- ethoxybenzamido) succinic acid | 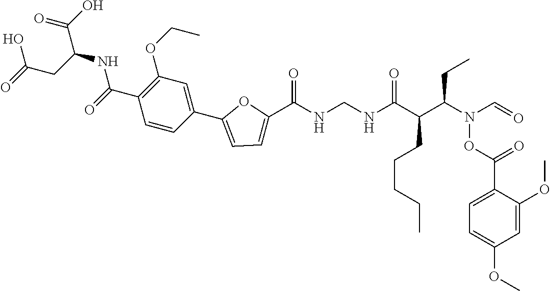 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1OC] | (S)-dibenzyl 2-(4- (5-((((R)-2-((R)-1- (N-((2,4- dimethoxybenzoyl) oxy)formamido) propyl) heptanamido) methyl)carbamoyl) furan-2-yl)-2- ethoxybenzamido) succinate |
| 97 | (3-ethoxy-5-(5-((((R)- 2-((R)-1-(N-((2- methyl-4- morpholinobenzoyl) oxy)formamido) propyl)heptanamido) methyl)carbamoyl) furan-2- yl)phenyl)phosphonic acid, 2Ammonia salt | 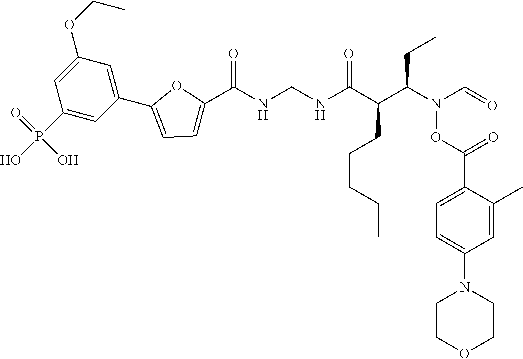 [SMILES: CCCCC[C@@H](C(=O)NCNC(=O)c1ccc(-c2cc(OCC)cc(P(=O)(O)O)c2)o1)[C@@H](CC)N(C=O)OC(=O)c1ccc(N2CCOCC2)cc1C] | dibenzyl (3- ethoxy-5-(5-((((R)- 2-((R)-1-(N-((2- methyl-4- morpholino- benzoyl)oxy) formamido) propyl) heptanamido) methyl)carbamoyl) furan-2- yl)phenyl) phosphonate |
| 98 | (S)-2-(2-ethoxy-4-(5- ((((R)-2-((R)-1-(N-((2- isopropylbenzoyl)oxy) formamido)propyl) heptanamido)methyl) carbamoyl)furan-2- yl)benzamido) succinic acid | 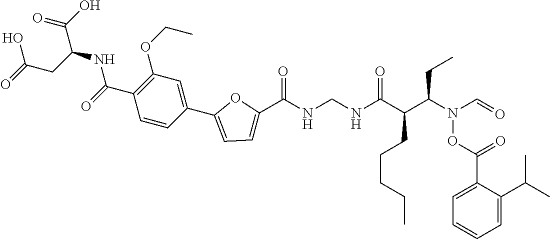 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccccc1C(C)C] | (S)-dibenzyl 2-(2- ethoxy-4-(5-((((R)- 2-((R)-1-(N-((2- isopropylbenzoyl) oxy)formamido) propyl) heptanamido) methyl)carbamoyl) furan-2- yl)benzamido) succinate |
| 99 | (S)-2-(4-(5-((((R)-2- ((R)-1-(N-((2,4- dimethylnicotinoyl) oxy)formamido) propyl)heptanamido) methyl)carbamoyl) furan-2-yl)-2- ethoxybenzamido) succinic acid | 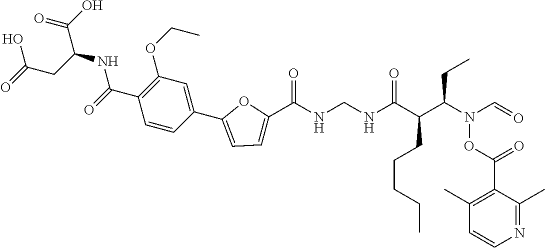 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1c(C)ccnc1C] | (S)-dibenzyl 2-(4- (5-((((R)-2-((R)-1- (N-((2,4- dimethyl- nicotinoyl) oxy)formamido) propyl) heptanamido) methyl)carbamoyl) furan-2-yl)-2- ethoxybenzamido) succinate |
| 100 | ethyl 4-(5-((((R)-2- ((R)-1-(N- hydroxyformamido) propyl)heptanamido) methyl)carbamoyl) furan-2-yl)benzoate | 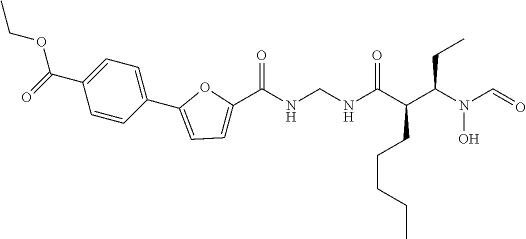 [SMILES: CCCCC[C@@H](C(=O)NCNC(=O)c1ccc(-c2ccc(C(=O)OCC)cc2)o1)[C@@H](CC)N(O)C=O] | ethyl 4-(5-((((R)-2- ((R)-1-(N- (benzyloxy) formamido)propyl) heptanamido) methyl) carbamoyl) furan-2- yl)benzoate |
| 101 | butyl 4-(5-((((R)-2- ((R)-1-(N- hydroxyformamido) propyl) heptanamido) methyl)carbamoyl) furan-2-yl)benzoate | 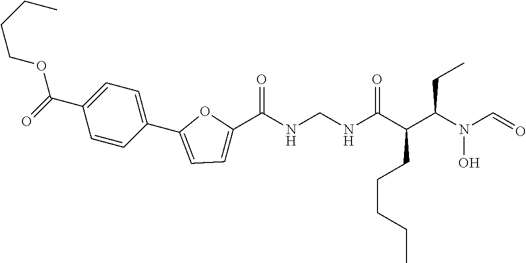 [SMILES: CCCCC[C@@H](C(=O)NCNC(=O)c1ccc(-c2ccc(C(=O)OCCCC)cc2)o1)[C@@H](CC)N(O)C=O] | butyl 4-(5-((((R)-2- ((R)-1-(N- (benzyloxy) formamido) propyl) heptanamido) methyl) carbamoyl)furan-2- yl)benzoate |
| 102 | phenethyl 4-(5-((((R)- 2-((R)-1-(N- hydroxyformamido) propyl) heptanamido) methyl)carbamoyl) furan-2-yl)benzoate | 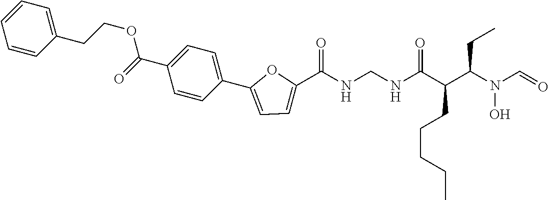 [SMILES: CCCCC[C@@H](C(=O)NCNC(=O)c1ccc(-c2ccc(C(=O)OCCc3ccccc3)cc2)o1)[C@@H](CC)N(O)C=O] | phenethyl 4-(5- ((((R)-2-((R)-1-(N- (benzyloxy) formamido) propyl) heptanamido) methyl)carbamoyl) furan-2-yl) benzoate |
| 103 | 2-morpholinoethyl 4- (5-((((R)-2-((R)-1-(N- hydroxyformamido) propyl) heptanamido) methyl)carbamoyl) furan-2-yl)benzoate | 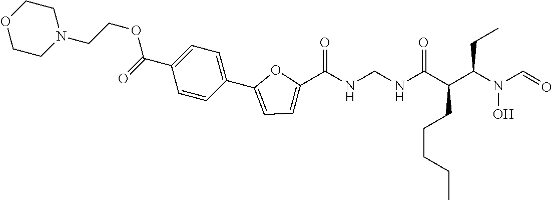 [SMILES: CCCCC[C@@H](C(=O)NCNC(=O)c1ccc(-c2ccc(C(=O)OCCN3CCOCC3)cc2)o1)[C@@H](CC)N(O)C=O] | 2-morpholinoethyl 4-(5-((((R)-2-((R)- 1-(N- (benzyloxy) formamido) propyl) heptanamido) methyl)carbamoyl) furan-2- yl)benzoate |
| 104 | 2-(dimethylamino) ethyl 4-(5-((((R)-2- ((R)-1-(N- hydroxyformamido) propyl)heptanamido) methyl)carbamoyl) furan-2-yl)benzoate | 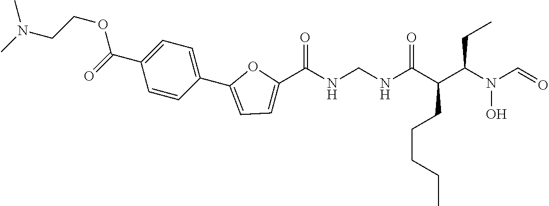 [SMILES: CCCCC[C@@H](C(=O)NCNC(=O)c1ccc(-c2ccc(C(=O)OCCN(C)C)cc2)o1)[C@@H](CC)N(O)C=O] | 2-(dimethyl- amino)ethyl 4-(5-((((R)-2- ((R)-1-(N- (benzyloxy) formamido) propyl) heptanamido) methyl)carbamoyl) furan-2- yl)benzoate |
| 105 | 2-(dimethylamino)-2- oxoethyl 4-(5-((((R)- 2-((R)-1-(N- hydroxyformamido) propyl)heptanamido) methyl)carbamoyl) furan-2-yl)benzoate | 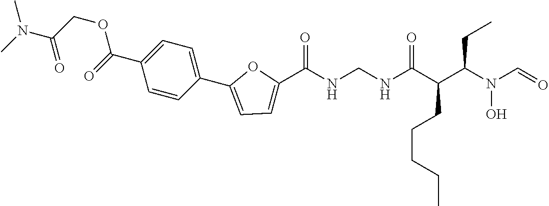 [SMILES: CCCCC[C@@H](C(=O)NCNC(=O)c1ccc(-c2ccc(C(=O)OCC(=O)N(C)C)cc2)o1)[C@@H](CC)N(O)C=O] | 2-(dimethyl- amino)- 2-oxoethyl 4-(5- ((((R)-2-((R)-1- (N-(benzyloxy) formamido) propyl) heptanamido) methyl) carbamoyl) furan-2- yl)benzoate |
| 106 | (S)-3-(2-ethoxy-4-(5- ((((R)-2-((R)-1-(N- hydroxyformamido) propyl)heptanamido) methyl)carbamoyl) furan-2-yl) benzamido)-4- ((isobutyryloxy) methoxy)-4- oxobutanoic acid | 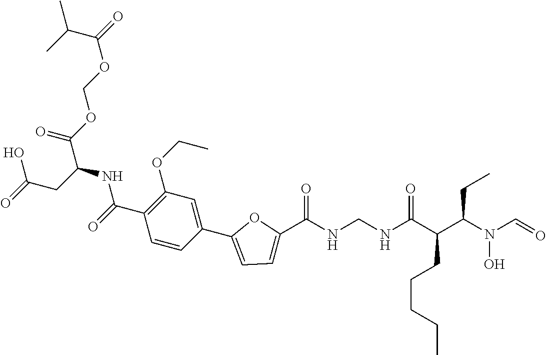 [SMILES: CCCCC[C@@H](C(=O)NCNC(=O)c1ccc(-c2ccc(C(=O)N[C@@H](CC(=O)O)C(=O)OCOC(=O)C(C)C)c(OCC)c2)o1)[C@@H](CC)N(O)C=O] | (S)-4-benzyl 1- ((isobutyryloxy) methyl) 2-(4-(5- ((((R)-2-((R)-1-(N- (benzyloxy) formamido) propyl) heptanamido) methyl)carbamoyl) furan-2-yl)-2- ethoxybenzamido) succinate |
| 107 | (((3-ethoxy-5-(5- ((((R)-2-((R)-1-(N- hydroxyformamido) propyl)heptanamido) methyl)carbamoyl) furan-2- yl)phenyl)phosphoryl) bis(oxy))bis (methylene) diisopropyl dicarbonate | 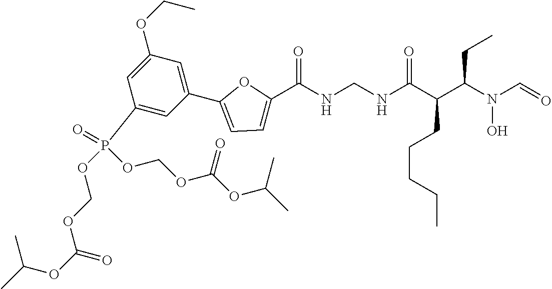 [SMILES: CCCCC[C@@H](C(=O)NCNC(=O)c1ccc(-c2cc(OCC)cc(P(=O)(OCOC(=O)OC(C)C)OCOC(=O)OC(C)C)c2)o1)[C@@H](CC)N(O)C=O] | (((3-(5-((((R)-2- ((R)-1-(N- (benzyloxy) formamido) propyl) heptanamido) methyl) carbamoyl) furan-2-yl)-5- ethoxyphenyl) phosphoryl)bis (oxy))bis (methylene) diisopropyl dicarbonate |
| 108 | diphenyl (3-ethoxy-5- (5-((((R)-2-((R)-1-(N- hydroxyformamido) propyl)heptanamido) methyl)carbamoyl) furan-2- yl)phenyl) phosphonate | 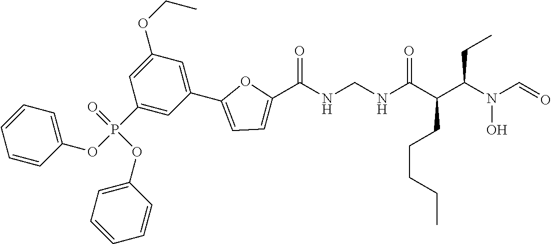 [SMILES: CCCCC[C@@H](C(=O)NCNC(=O)c1ccc(-c2cc(OCC)cc(P(=O)(Oc3ccccc3)Oc3ccccc3)c2)o1)[C@@H](CC)N(O)C=O] | diphenyl (3-(5- ((((R)-2-((R)-1-(N- (benzyloxy) formamido)propyl) heptanamido) methyl) carbamoyl) furan-2-yl)-5- ethoxyphenyl) phosphonate |
| 109 | (((3-ethoxy-5-(5- ((((R)-2-((R)-1-(N- hydroxyformamido) propyl)heptanamido) methyl)carbamoyl) furan-2- yl)phenyl)phosphoryl) bis(oxy))bis (methylene) bis(2- methylpropanoate) | 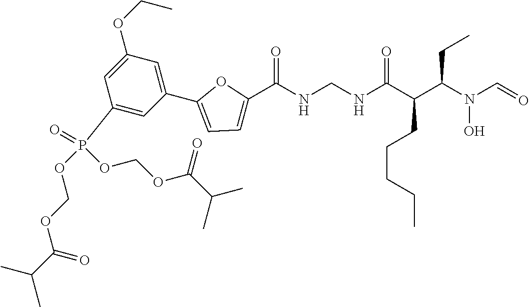 [SMILES: CCCCC[C@@H](C(=O)NCNC(=O)c1ccc(-c2cc(OCC)cc(P(=O)(OCOC(=O)C(C)C)OCOC(=O)C(C)C)c2)o1)[C@@H](CC)N(O)C=O] | (((3-(5-((((R)-2- ((R)-1-(N- (benzyloxy) formamido) propyl) heptanamido) methyl) carbamoyl)furan- 2-yl)-5- ethoxyphenyl) phosphoryl)bis (oxy))bis (methylene) bis(2- methylpropanoate |
| 110 | (S)-3-(2-ethoxy-4-(5- ((((R)-2-((R)-1-(N- hydroxyformamido) propyl)heptanamido) methyl)carbamoyl) furan-2-yl) benzamido)-4-oxo-4- phenoxybutanoic acid | 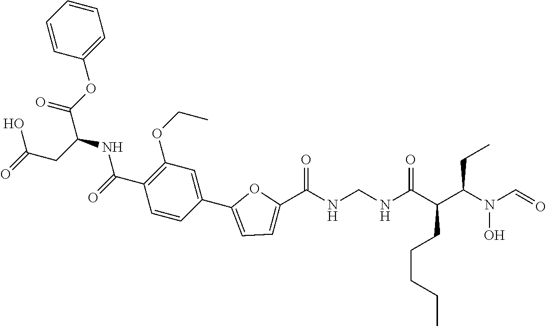 [SMILES: CCCCC[C@@H](C(=O)NCNC(=O)c1ccc(-c2ccc(C(=O)N[C@@H](CC(=O)O)C(=O)Oc3ccccc3)c(OCC)c2)o1)[C@@H](CC)N(O)C=O] | (S)-4-benzyl 1- phenyl 2-(4-(5- ((((R)-2-((R)-1-(N- (benzyloxy) formamido)propyl) heptanamido) methyl) carbamoyl)furan- 2-yl)-2- ethoxybenzamido) succinate |
| 111 | (S)- bis((isobutyryloxy) methyl) 2-(2-ethoxy-4- (5-((((R)-2-((R)-1- (N-hydroxy- formamido) propyl)heptanamido) methyl)carbamoyl) furan-2- yl)benzamido) succinate | 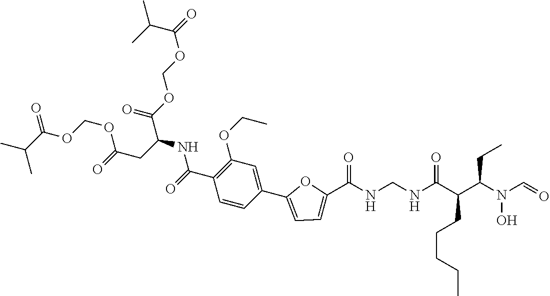 [SMILES: CCCCC[C@@H](C(=O)NCNC(=O)c1ccc(-c2ccc(C(=O)N[C@@H](CC(=O)OCOC(=O)C(C)C)C(=O)OCOC(=O)C(C)C)c(OCC)c2)o1)[C@@H](CC)N(O)C=O] | (S)- bis((isobutyryloxy) methyl) 2-(4-(5- ((((R)-2-((R)-1-(N- (benzyloxy) formamido) propyl) heptanamido) methyl) carbamoyl)furan- 2-yl)-2- ethoxybenzamido) succinate |
| 112 | (S)-di-tert-butyl 2-(2- ethoxy-4-(5-((((R)-2- ((R)-1-(N- hydroxyformamido) propyl)heptanamido) methyl)carbamoyl) furan-2- yl)benzamido) succinate | 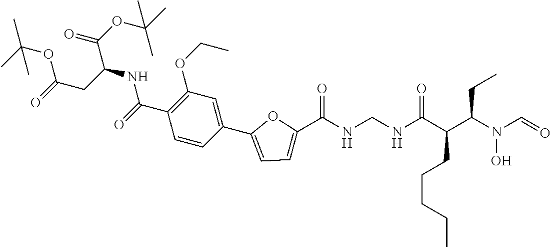 [SMILES: CCCCC[C@@H](C(=O)NCNC(=O)c1ccc(-c2ccc(C(=O)N[C@@H](CC(=O)OC(C)(C)C)C(=O)OC(C)(C)C)c(OCC)c2)o1)[C@@H](CC)N(O)C=O] | (S)-di-tert-butyl 2- (4-(5-((((R)-2-((R)- 1-(N- (benzyloxy) formamido) propyl) heptanamido) methyl) carbamoyl) furan-2-yl)-2- ethoxybenzamido) succinate |
| 113 | (S)-4-ethoxy-3-(2- ethoxy-4-(5-((((R)-2- ((R)-1-(N- hydroxyformamido) propyl)heptanamido) methyl)carbamoyl) furan-2-yl) benzamido)-4- oxobutanoic acid | 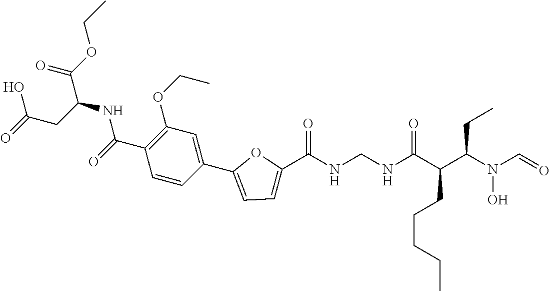 [SMILES: CCCCC[C@@H](C(=O)NCNC(=O)c1ccc(-c2ccc(C(=O)N[C@@H](CC(=O)O)C(=O)OCC)c(OCC)c2)o1)[C@@H](CC)N(O)C=O] | (S)-4-benzyl 1- ethyl 2-(2-ethoxy- 4-(5-((((R)-2-((R)- 1-(N- hydroxy- formamido) propyl) heptanamido) methyl)carbamoyl) furan-2- yl)benzamido) succinate |
| 114 | (S)-4-(tert-butoxy)-3- (2-ethoxy-4-(5-((((R)- 2-((R)-1-(N- hydroxyformamido) propyl)heptanamido) methyl)carbamoyl) furan-2-yl) benzamido)-4- oxobutanoic acid | 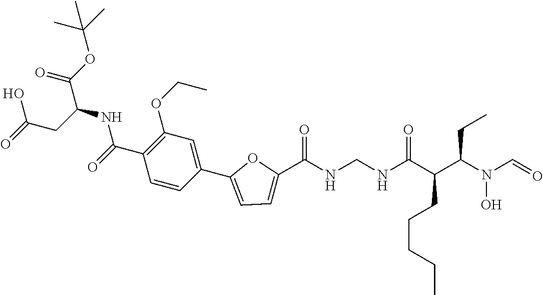 [SMILES: CCCCC[C@@H](C(=O)NCNC(=O)c1ccc(-c2ccc(C(=O)N[C@@H](CC(=O)O)C(=O)OC(C)(C)C)c(OCC)c2)o1)[C@@H](CC)N(O)C=O] | (S)-4-benzyl 1-tert- butyl 2-(4-(5- ((((R)-2-((R)-1-(N- (benzyloxy) formamido) propyl) heptanamido) methyl) carbamoyl)furan- 2-yl)-2- ethoxybenzamido) succinate |
| 115 | (((3-ethoxy-5-(5- ((((R)-2-((R)-1-(N- hydroxyformamido) propyl)heptanamido) methyl)carbamoyl) furan-2- yl)phenyl) phosphoryl) bis(oxy))bis (methylene) diacetate | 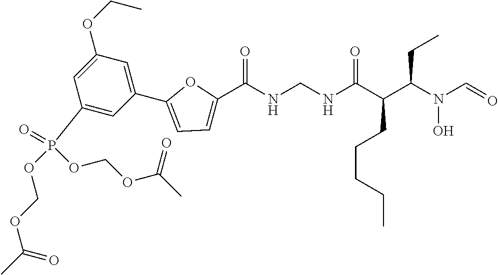 [SMILES: CCCCC[C@@H](C(=O)NCNC(=O)c1ccc(-c2cc(OCC)cc(P(=O)(OCOC(C)=O)OCOC(C)=O)c2)o1)[C@@H](CC)N(O)C=O] | (((3-(5-((((R)-2- ((R)-1-(N- (benzyloxy) formamido) propyl) heptanamido) methyl) carbamoyl)furan- 2-yl)-5- ethoxyphenyl) phosphoryl)bis (oxy))bis (methylene) diacetate |
| 116 | (S)-4- ((benzoyloxy) methoxy)-3-(2- ethoxy-4-(5-((((R)-2- ((R)-1-(N- hydroxyformamido) propyl)heptanamido) methyl)carbamoyl) furan-2-yl) benzamido)-4- oxobutanoic acid | 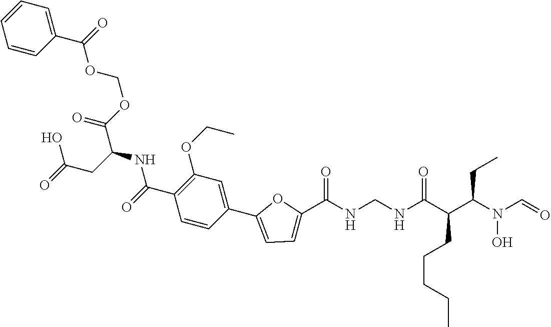 [SMILES: CCCCC[C@@H](C(=O)NCNC(=O)c1ccc(-c2ccc(C(=O)N[C@@H](CC(=O)O)C(=O)OCOC(=O)c3ccccc3)c(OCC)c2)o1)[C@@H](CC)N(O)C=O] | (S)-1- ((benzoyloxy) methyl) 4-benzyl 2-(4-(5-((((R)-2- ((R)-1-(N- (benzyloxy) formamido) propyl) heptanamido) methyl) carbamoyl) furan-2-yl)-2- ethoxybenzamido) succinate |
| 117 | (S)-2-(2-ethoxy-4-(5- ((((R)-2-((R)-1-(N- ((4-fluoro-2- methylbenzoyl)oxy) formamido)propyl) heptanamido)methyl) carbamoyl)furan-2- yl)benzamido) succinic acid | 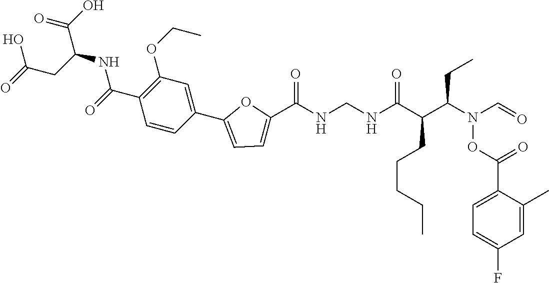 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(F)cc1C] | (S)-dibenzyl 2-(2- ethoxy-4-(5-((((R)- 2-((R)-1-(N-((4- fluoro-2- methylbenzoyl) oxy)formamido) propyl) heptanamido) methyl) carbamoyl)furan- 2-yl)benzamido) succinate |
| 118 | (S)-2-(2-ethoxy-4-(5- ((((R)-2-((R)-1-(N- ((piperidine-4- carbonyl)oxy) formamido)propyl) heptanamido)methyl) carbamoyl)furan-2- yl)benzamido) succinic acid, Trifluoroacetic acid salt | 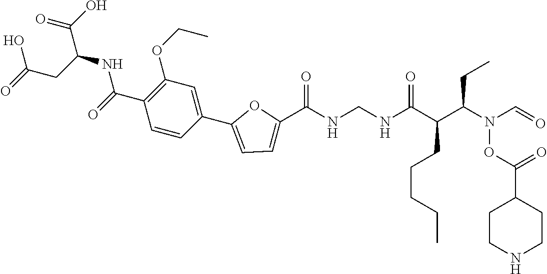 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)C1CCNCC1] | (S)-dibenzyl 2-(2- ethoxy-4-(5-((((R)- 2-((R)-1-(N- ((piperidine-4- carbonyl)oxy) formamido) propyl) heptanamido) methyl) carbamoyl) furan-2-yl) benzamido) succinate, Trifluoroacetic acid salt |
| 119 | (S)-2-(2-ethoxy-4-(5- ((((R)-2-((R)-1-(N- ((4-(trifluoromethyl) benzoyl)oxy) formamido)propyl) heptanamido) methyl)carbamoyl) furan-2- yl)benzamido) succinic acid | 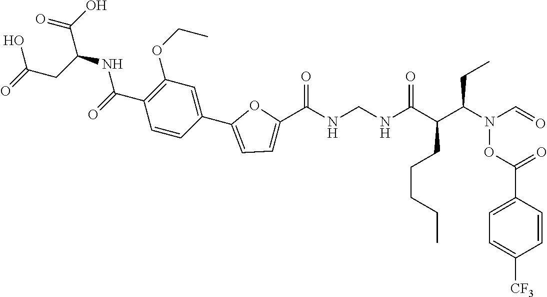 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(C(F)(F)F)cc1] | (S)-dibenzyl 2-(2- ethoxy-4-(5-((((R)- 2-((R)-1-(N-((4- (trifluoromethyl) benzoyl)oxy) formamido) propyl) heptanamido) methyl) carbamoyl) furan-2- yl)benzamido) succinate |
| 120 | (S)-2-(2-ethoxy-4-(5- ((((R)-2-((R)-1-(N- ((4-nitrobenzoyl)oxy) formamido)propyl) heptanamido) methyl)carbamoyl) furan-2-yl) benzamido) succinic acid | 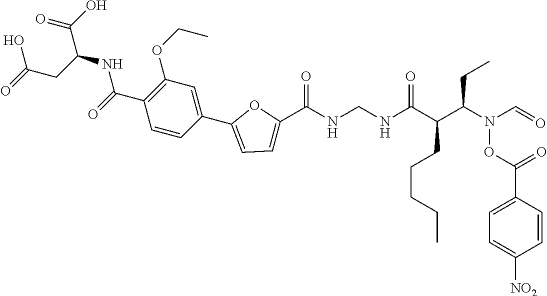 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc([N+](=O)[O-])cc1] | (S)-dibenzyl 2-(2- ethoxy-4-(5-((((R)- 2-((R)-1-(N-((4- nitrobenzoyl)oxy) formamido)propyl) heptanamido) methyl)carbamoyl) furan-2- yl)benzamido) succinate |
| 121 | (S)-2-(2-ethoxy-4-(5- ((((R)-2-((R)-1-(N- ((4-hydroxybenzoyl) oxy)formamido) propyl)heptanamido) methyl)carbamoyl) furan-2- yl)benzamido) succinic acid | 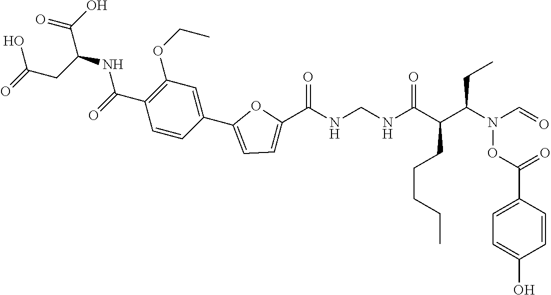 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(O)cc1] | (S)-dibenzyl 2-(4- (5-((((R)-2-((R)-1- (N-((4- acetoxybenzoyl) oxy)formamido) propyl) heptanamido) methyl)carbamoyl) furan-2-yl)-2- ethoxybenzamido) succinate |
| 122 | (S)-2-(2-ethoxy-4-(5- ((((R)-2-((R)-1-(N- ((2-fluoro-4- methoxybenzoyl)oxy) formamido)propyl) heptanamido)methyl) carbamoyl)furan-2- yl)benzamido) succinic acid | 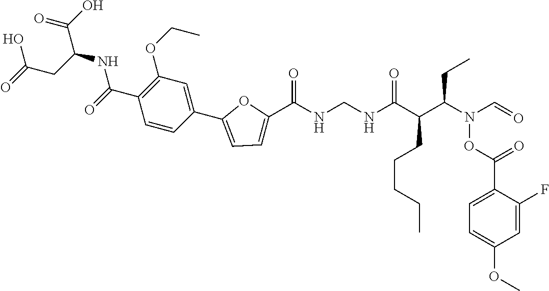 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1F] | (S)-dibenzyl 2-(2- ethoxy-4-(5-((((R)- 2-((R)-1-(N-((2- fluoro-4- methoxybenzoyl) oxy)formamido) propyl) heptanamido) methyl)carbamoyl) furan-2- yl)benzamido) succinate |
| 123 | (S)-2-(2-ethoxy-4-(5- ((((R)-2-((R)-1-(N- ((4-methoxy-2- (trifluoromethyl) benzoyl)oxy) formamido)propyl) heptanamido)methyl) carbamoyl)furan-2- yl)benzamido) succinic acid | 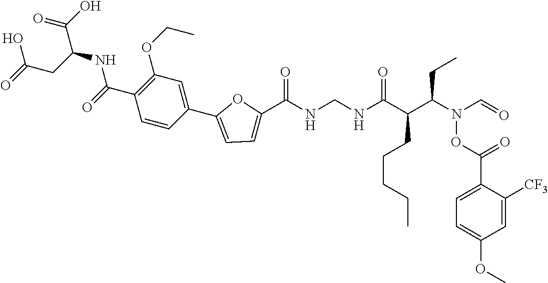 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1C(F)(F)F] | (S)-dibenzyl 2-(2- ethoxy-4-(5-((((R)- 2-((R)-1-(N-((4- methoxy-2- (trifluoromethyl) benzoyl)oxy) formamido) propyl) heptanamido) methyl)carbamoyl) furan-2-yl) benzamido) succinate |
| 124 | (S)-2-(2-ethoxy-4-(5- ((((R)-2-((R)-1-(N- ((4-methoxy-2- (trifluoromethoxy) benzoyl)oxy) formamido)propyl) heptanamido) methyl)carbamoyl) furan-2- yl)benzamido) succinic acid | 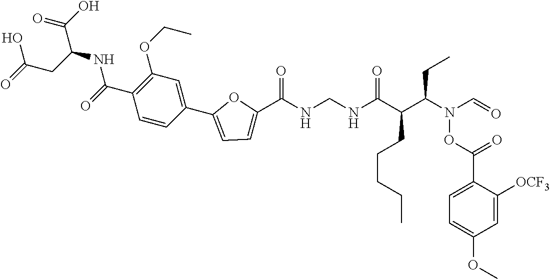 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1OC(F)(F)F] | (S)-dibenzyl 2-(2- ethoxy-4-(5-((((R)- 2-((R)-1-(N-((4- methoxy-2- (trifluoromethoxy) benzoyl)oxy) formamido) propyl) heptanamido) methyl) carbamoyl)furan-2- yl)benzamido) succinate |
| 125 | ((4-(5-((((R)-2-((R)- 1-(N-(benzoyloxy) formamido)propyl) heptanamido) methyl)carbamoyl) furan-2-yl)-2- ethoxybenzamido) methyl)phosphonic Acid | 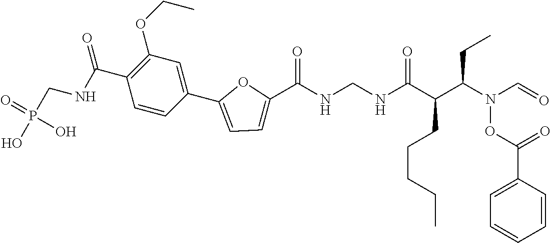 [SMILES: CCCCC[C@@H](C(=O)NCNC(=O)c1ccc(-c2ccc(C(=O)NCP(=O)(O)O)c(OCC)c2)o1)[C@@H](CC)N(C=O)OC(=O)c1ccccc1] | dibenzyl ((4-(5- ((((R)-2-((R)-1-(N- (benzoyloxy) formamido) propyl) heptanamido) methyl)carbamoyl) furan-2-yl)-2- ethoxybenzamido) methyl) phosphonate |
| 126 | (S)-2-(2-ethoxy-4-(5- ((((R)-2-((R)-1-(N- ((2-methyl-4- (trifluoromethyl) benzoyl)oxy) formamido)propyl) heptanamido) methyl)carbamoyl) furan-2- yl)benzamido) succinic acid | 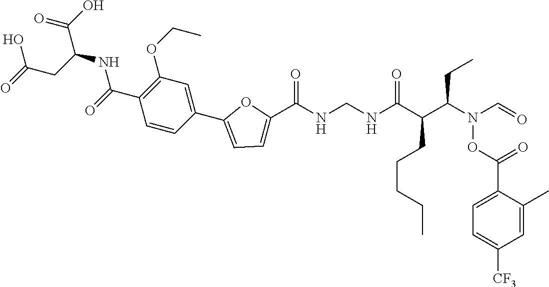 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(C(F)(F)F)cc1C] | (S)-dibenzyl 2-(2- ethoxy-4-(5-((((R)- 2-((R)-1-(N-((2- methyl-4- (trifluoromethyl) benzoyl)oxy) formamido) propyl) heptanamido) methyl)carbamoyl) furan-2- yl)benzamido) succinate |
| 127 | (S)-2-(4-(5-((((R)-2- ((R)-1-(N-((4-(4H- 1,2,4-triazol-4- yl)benzoyl)oxy) formamido)propyl) heptanamido)methyl) carbamoyl) furan-2-yl)-2- ethoxybenzamido) succinic acid | 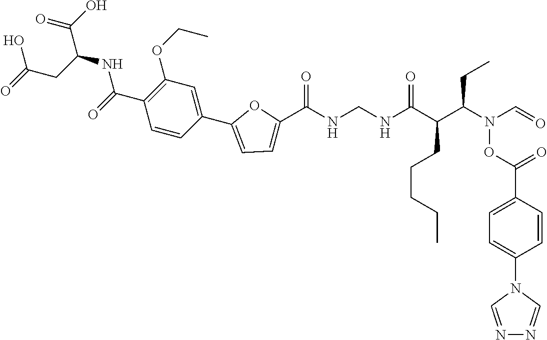 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(-n2cnnc2)cc1] | (S)-dibenzyl 2-(4- (5-((((R)-2-((R)-1- (N-((4-(4H-1,2,4- triazol-4- yl)benzoyl)oxy) formamido) propyl) heptanamido) methyl) carbamoyl) furan-2-yl)-2- ethoxybenzamido) succinate |
| 128 | (S)-2-(4-(5-((((R)-2- ((R)-1-(N-((3-(1H- pyrrol-1- yl)benzoyl)oxy) formamido)propyl) heptanamido) methyl)carbamoyl) furan-2-yl)-2- ethoxybenzamido) succinic acid | 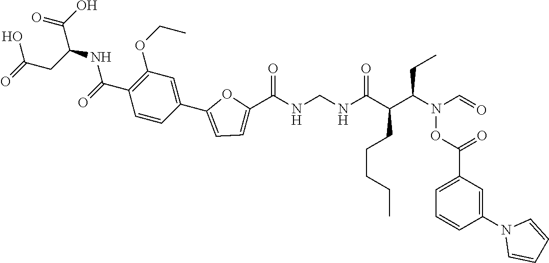 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1cccc(-n2cccc2)c1] | (S)-dibenzyl 2-(4- (5-((((R)-2-((R)-1- (N-((3-(1H- pyrrol-1- yl)benzoyl)oxy) formamido)propyl) heptanamido) methyl) carbamoyl) furan-2-yl)-2- ethoxybenzamido) succinate |
| 129 | (S)-2-(2-ethoxy-4-(5- ((((R)-2-((R)-1-(N- ((4-(N- methylmethyl- sulfonamido)benzoyl) oxy)formamido) propyl)heptanamido) methyl)carbamoyl) furan-2-yl) benzamido) succinic acid | 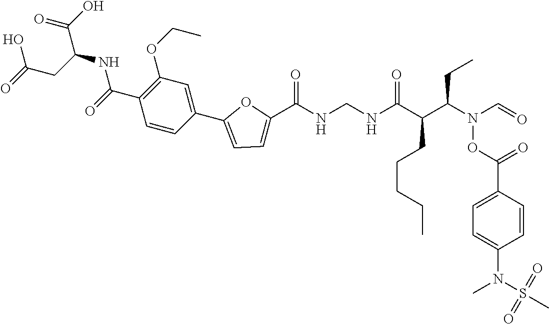 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(N(C)S(C)(=O)=O)cc1] | (S)-dibenzyl 2-(2- ethoxy-4-(5-((((R)- 2-((R)-1-(N-((4- (N-methylmethyl- sulfonamido) benzoyl)oxy) formamido) propyl) heptanamido) methyl)carbamoyl) furan-2- yl)benzamido) succinate |
| 130 | (S)-2-(4-(5-((((R)-2- ((R)-1-(N-((4-(1H- pyrazol-1- yl)benzoyl)oxy) formamido)propyl) heptanamido)methyl) carbamoyl) furan-2-yl)-2- ethoxybenzamido) succinic acid | 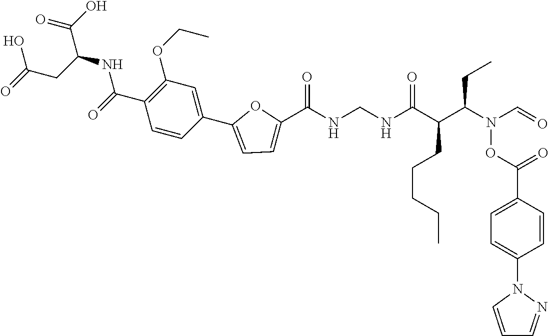 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(-n2cccn2)cc1] | (S)-dibenzyl 2-(4- (5-((((R)-2-((R)-1- (N-((4-(1H- pyrazol-1- yl)benzoyl)oxy) formamido) propyl) heptanamido) methyl) carbamoyl) furan-2-yl)-2- ethoxybenzamido) succinate |
| 131 | (S)-2-(4-(5-((((R)-2- ((R)-1-(N-((4-(1H- imidazol-1- yl)benzoyl)oxy) formamido)propyl) heptanamido)methyl) carbamoyl) furan-2-yl)-2- ethoxybenzamido) succinic acid | 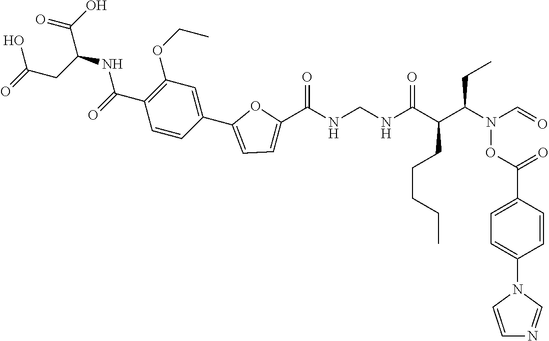 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(-n2ccnc2)cc1] | (S)-dibenzyl 2-(4- (5-((((R)-2-((R)-1- (N-((4-(1H- imidazol-1- yl)benzoyl)oxy) formamido)propyl) heptanamido) methyl) carbamoyl) furan-2-yl)-2- ethoxybenzamido) succinate |
| 132 | ((2-ethoxy-4-(5- ((((R)-2-((R)-1-(N- ((4-methoxybenzoyl) oxy)formamido) propyl)heptanamido) methyl)carbamoyl) furan-2- yl)benzamido) methyl) phosphonic acid | 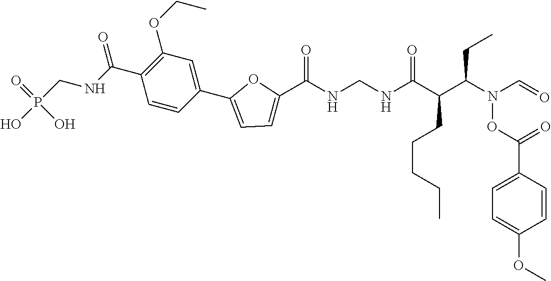 [SMILES: CCCCC[C@@H](C(=O)NCNC(=O)c1ccc(-c2ccc(C(=O)NCP(=O)(O)O)c(OCC)c2)o1)[C@@H](CC)N(C=O)OC(=O)c1ccc(OC)cc1] | dibenzyl ((2- ethoxy-4-(5-((((R)- 2-((R)-1-(N-((4- methoxybenzoyl) oxy)formamido) propyl) heptanamido) methyl)carbamoyl) furan-2- yl)benzamido) methyl) phosphonate |
| 133 | ((2-ethoxy-4-(5-((((R)- 2-((R)-1-(N-((4- methoxy-2- methylbenzoyl)oxy) formamido)propyl) heptanamido)methyl) carbamoyl)furan-2- yl)benzamido)methyl) phosphonic acid | 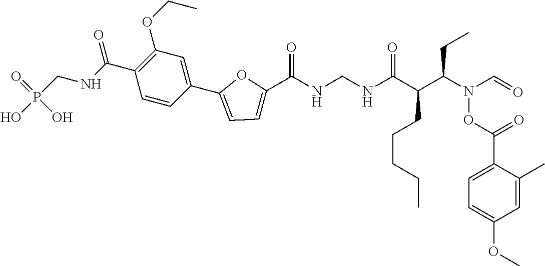 [SMILES: CCCCC[C@@H](C(=O)NCNC(=O)c1ccc(-c2ccc(C(=O)NCP(=O)(O)O)c(OCC)c2)o1)[C@@H](CC)N(C=O)OC(=O)c1ccc(OC)cc1C] | dibenzyl ((2- ethoxy-4-(5- ((((R)-2-((R)- 1-(N-((4- methoxy-2- methylbenzoyl) oxy)formamido) propyl) heptanamido) methyl) carbamoyl) furan-2- yl)benzamido) methyl) phosphonate |
| 134 | ((2-ethoxy-4-(5-((((R)- 2-((R)-1-(N-((2- methyl-4- (morpholinomethyl) benzoyl)oxy) formamido)propyl) heptanamido) methyl)carbamoyl) furan-2- yl)benzamido)methyl) phosphonic acid, Trifluoroacetic acid salt | 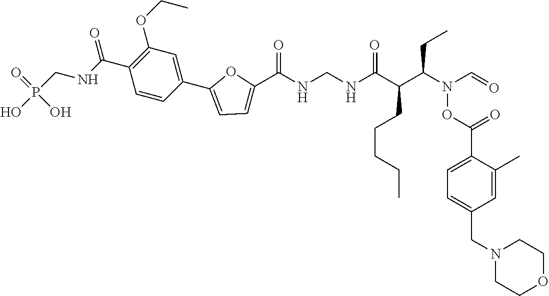 [SMILES: CCCCC[C@@H](C(=O)NCNC(=O)c1ccc(-c2ccc(C(=O)NCP(=O)(O)O)c(OCC)c2)o1)[C@@H](CC)N(C=O)OC(=O)c1ccc(CN2CCOCC2)cc1C] | dibenzyl ((2- ethoxy-4-(5- ((((R)- 2-((R)-1-(N-((2- methyl-4- (morpholino- methyl)benzoyl) oxy)formamido) propyl) heptanamido) methyl)carbamoyl) furan-2- yl)benzamido) methyl) phosphonate |
| 135 | (S)-2-(2-ethoxy-4-(5- ((((R)-2-((R)-1-(N- ((4-(2-(2- hydroxyethoxy) ethoxy)benzoyl)oxy) formamido)propyl) heptanamido)methyl) carbamoyl)furan-2- yl)benzamido) succinic acid | 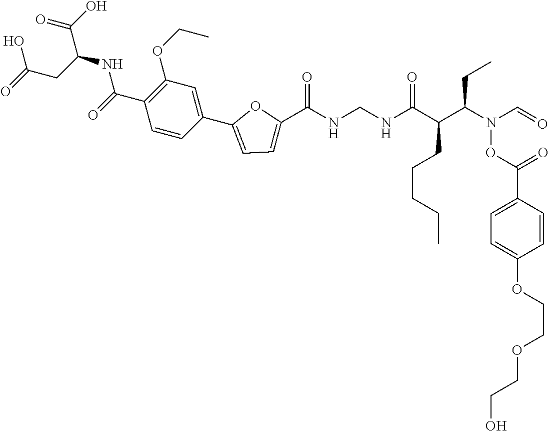 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OCCOCCO)cc1] | (S)-dibenzyl 2-(4- (5-((((R)-2-((R)-1- (N-((4-(2-(2- (benzyloxy)ethoxy) ethoxy)benzoyl) oxy)formamido) propyl) heptanamido) methyl)carbamoyl) furan-2-yl)-2- ethoxybenzamido) succinate |

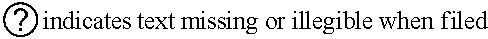

Particular preparations of the compounds of Examples 51, 54, and 78-80:

### Example 51

### (S)-2-(2-ethoxy-4-(5-((((R)-2-((R)-1-(N-((4-methoxybenzoyl)oxy)formamido)propyl)heptanamido)methyl)carbamoyl)furan-2-yl)benzamido)succinic Acid

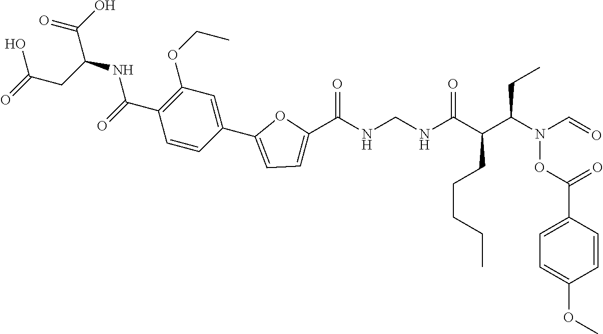 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1]

10% Pd—C (13.93 mg, 0.013 mmol) was added to a solution of (S)-dibenzyl 2-(2-ethoxy-4-(5-((((R)-2-((R)-1-(N-((4-methoxybenzoyl)oxy)formamido)propyl)heptanamido)methyl)carbamoyl)furan-2-yl)benzamido)succinate (124 mg, 0.131 mmol) in DCM (218 μl). MeOH (1091 μl) was added under N₂. The reaction vial was evacuated and back-filled with N₂ (3×) and was then evacuated and back-filled with H₂ (3×). The mixture was hydrogenated under balloon for 20 min. The mixture was evacuated and back-filled with N₂ (3×), filtered through a syringe filter washing with MeOH and concentrated. Purification on reverse phase HPLC afforded the title compound as an orange solid. (37 mg, 35% yield)

### Example 54

### (S)-2-(2-ethoxy-4-(5-((((R)-2-((R)-1-(N-((4-methoxy-2-methylbenzoyl)oxy)formamido)propyl)heptanamido)methyl)carbamoyl)furan-2-yl)benzamido)succinic Acid, 2Ammonia Salt

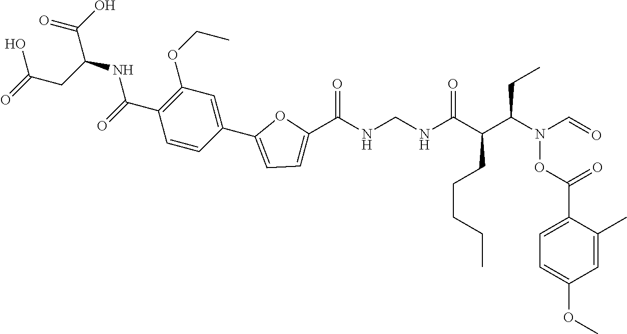 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1C]

Pd/C (11.07 mg, 10.41 μmol) was added to a flask containing (S)-dibenzyl 2-(2-ethoxy-4-(5-((((R)-2-((R)-1-(N-((4-methoxy-2-methylbenzoyl)oxy)formamido)propyl)heptanamido)methyl)carbamoyl)furan-2-yl)benzamido)succinate (100 mg, 0.104 mmol) under N₂. EtOH (5 mL) was added and the reaction was purged with N₂ for 15 min. The reaction was then purged with H₂ (balloon) for 20 min and then stirred under H₂ (balloon) for 2 h. The reaction was purged with N₂ for 30 min before it was filtered through a pad of Celite. The filtrate was concentrated. Purification on basic reverse phase HPLC afforded the title compound as an colorless solid. (29 mg, 34% yield)

### Example 78

### (S)-2-(2-ethoxy-4-(5-((((R)-2-((R)-1-(N-((4-(2-oxopyrrolidin-1-yl)benzoyl)oxy)formamido)propyl)heptanamido)methyl)carbamoyl)furan-2-yl)benzamido)succinic Acid, 2Ammonia Salt

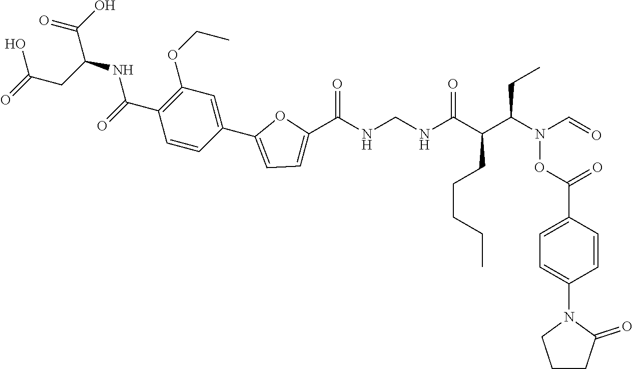 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(N2CCCC2=O)cc1]

Pd/C (10.64 mg, 10.00 μmol) was added to a flask containing (S)-dibenzyl 2-(2-ethoxy-4-(5-((((R)-2-((R)-1-(N-((4-(2-oxopyrrolidin-1-yl)benzoyl)oxy)formamido)propyl)heptanamido)methyl)carbamoyl)furan-2-yl)benzamido)succinate (100 mg, 0.100 mmol) under N₂. EtOH (5 mL) was added and the reaction was purged with N₂ for 15 min. The reaction was then purged with H₂ (balloon) for 20 min and then stirred under H₂ (balloon) for 2 h. The reaction was purged with N₂ for 30 min before it was filtered through a pad of Celite. The filtrate was concentrated. Purification on basic reverse phase HPLC afforded the title compound as an colorless solid. (31 mg, 36% yield)

### Example 79

### (S)-2-(4-(5-((((R)-2-((R)-1-(N-((4-(1H-pyrazol-1-yl)benzoyl)oxy)formamido)propyl)heptanamido)methyl)carbamoyl)furan-2-yl)-2-ethoxybenzamido)succinic Acid, 2Ammonia Salt

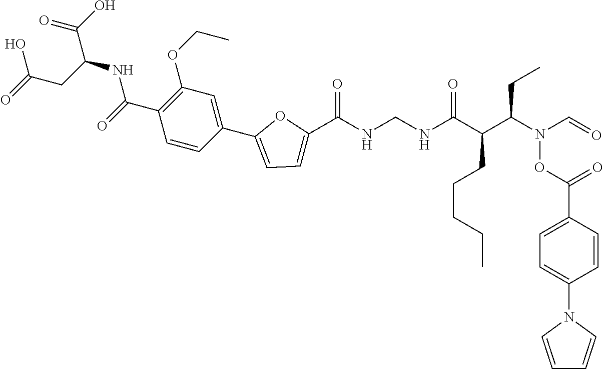 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(-n2cccc2)cc1]

Pd/C (8.5 mg, 8.00 μmol) was added to a flask containing (S)-dibenzyl 2-(4-(5-((((R)-2-((R)-1-(N-((4-(1H-pyrrol-1-yl)benzoyl)oxy)formamido)propyl)heptanamido)methyl)carbamoyl)furan-2-yl)-2-ethoxybenzamido)succinate (102 mg, 0.104 mmol) under N₂. EtOH (5 mL) was added and the reaction was purged with N₂ for 15 min. The reaction was then purged with H₂ (balloon) for 20 min and then stirred under H₂ (balloon) for 2 h. The reaction was purged with N₂ for 30 min before it was filtered through a pad of Celite. The filtrate was concentrated. Purification on basic reverse phase HPLC afforded the title compound as an colorless solid. (32 mg, 37% yield)

### Example 80

### (S)-2-(2-ethoxy-4-(5-((((R)-2-((R)-1-(N-((phosphonooxy)methoxy)formamido)propyl)heptanamido)methyl)carbamoyl)furan-2-yl)benzamido)succinic Acid, Ammonia Salt

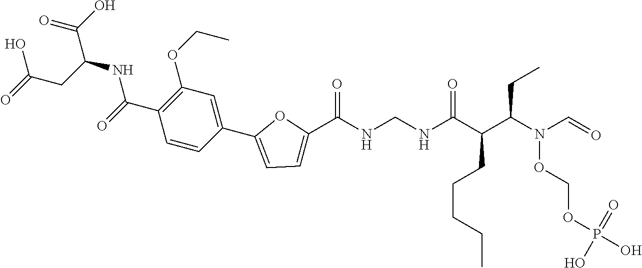 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OCOP(=O)(O)O]

A solution of (2S)-dibenzyl 2-(4-(5-((((2R)-2-((1R)-1-(N-((((benzyloxy)(hydroxy)phosphoryl)oxy)methoxy)formamido)propyl)heptanamido)methyl)carbamoyl)furan-2-yl)-2-ethoxybenzamido)succinate (572 mg, 0.565 mmol) and Pd—C (60.1 mg, 0.056 mmol) in MeOH (5646 μl) was vacuum pumped and backfilled with Nitrogen 3×. The Nitrogen was replaced with Hydrogen via a balloon. The reaction stirred for 1 h at RT. The reaction was vacuum pumped and backfilled with Nitrogen. The reaction was filtered through a pad of celite and concentrated. Purification on Si-C18 (0-50%, MeCN/0.1% NH₄OH in H2O/) afforded the title compound as a glass. (50 mg, 12% yield)

### Example 136

### 5-phenyl-N—(((R)-2-((R)-1-(N-(pivaloyloxy)formamido)propyl)heptanamido)methyl)furan-2-carboxamide

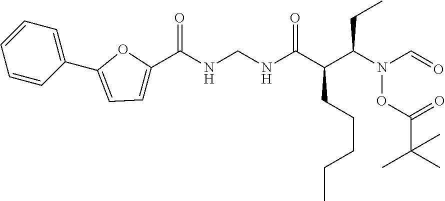 [SMILES: CCCCC[C@@H](C(=O)NCNC(=O)c1ccc(-c2ccccc2)o1)[C@@H](CC)N(C=O)OC(=O)C(C)(C)C]

Pivalic anhydride (709 μl, 3.49 mmol) was slowly added to a stirring solution of Et₃N (325 μl, 2.328 mmol) and N—(((R)-2-((R)-1-(N-hydroxyformamido)propyl)heptanamido)methyl)-5-phenylfuran-2-carboxamide (100 mg, 0.233 mmol) in CH₃CN (1.5 mL) at rt. The resulting mixture was stirred for 1 h and was then concentrated. Purification on Si (0-100% EtOAc/Hexanes) afforded the title compound as a colorless solid. (99 mg, 79% yield)

Examples 137-140 were prepared from the indicated intermediate by methods analogous to those described for Example 136 from the Intermediates listed in the table below. Example 137 was prepared using the acid chloride instead of the corresponding anhydride.

| Ex | Name | Structure | Intermediate |
|---|---|---|---|
| 137 | 5-phenyl-N-(((R)-2- ((R)-1-(N-(2- phenylacetoxy) formamido)propyl) heptanamido)methyl) furan-2-carboxamide | 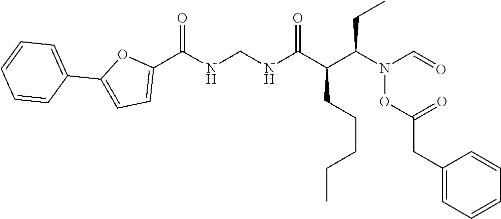 [SMILES: CCCCC[C@@H](C(=O)NCNC(=O)c1ccc(-c2ccccc2)o1)[C@@H](CC)N(C=O)OC(=O)Cc1ccccc1] | N-(((R)-2-((R)-1-(N- hydroxyformamido) propyl)heptanamido) methyl)-5-phenylfuran- 2-carboxamide |
| 138 | N-((6R,7R)-6-ethyl- 5-formyl-3,8-dioxo- 7-pentyl-2,4-dioxa- 5,9-diazadecan-10- yl)-5-phenylfuran- 2-carboxamide | 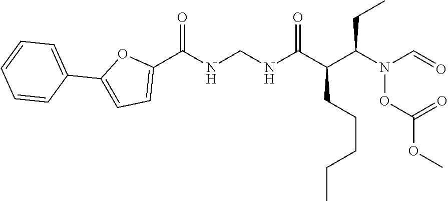 [SMILES: CCCCC[C@@H](C(=O)NCNC(=O)c1ccc(-c2ccccc2)o1)[C@@H](CC)N(C=O)OC(=O)OC] | N-(((R)-2-((R)-1-(N- hydroxyformamido) propyl)heptanamido) methyl)-5-phenylfuran- 2-carboxamide |
| 139 | N-((4R,5R)-5-ethyl- 6-formyl-10,10- dimethyl-3,8-dioxo- 4-pentyl-7,9-dioxa- 2,6-diazaundecyl)- 5-phenylfuran-2- carboxamide | 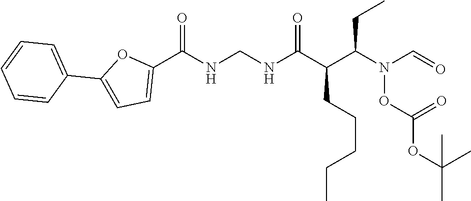 [SMILES: CCCCC[C@@H](C(=O)NCNC(=O)c1ccc(-c2ccccc2)o1)[C@@H](CC)N(C=O)OC(=O)OC(C)(C)C] | N-(((R)-2-((R)-1-(N- hydroxyformamido) propyl)heptanamido) methyl)-5-phenylfuran- 2-carboxamide |
| 140 | N-(((R)-2-((R)-1- (N-(benzoyloxy) formamido)propyl) heptanamido)methyl)- 5-phenylfuran-2- carboxamide | 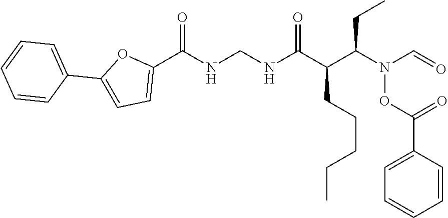 [SMILES: CCCCC[C@@H](C(=O)NCNC(=O)c1ccc(-c2ccccc2)o1)[C@@H](CC)N(C=O)OC(=O)c1ccccc1] | N-(((R)-2-((R)-1-(N- hydroxyformamido) propyl)heptanamido) methyl)-5-phenylfuran- 2-carboxamide |

### Example 141

### (3-ethoxy-5-(5-((((R)-2-((R)-1-(N-((5-methyl-2-oxo-1,3-dioxol-4-yl)methoxy)formamido)propyl)heptanamido)methyl)carbamoyl)furan-2-yl)phenyl)phosphonic Acid

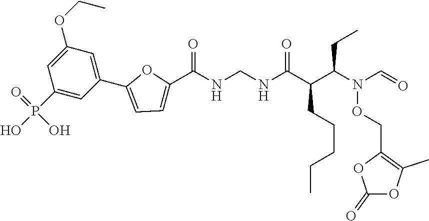 [SMILES: CCCCC[C@@H](C(=O)NCNC(=O)c1ccc(-c2cc(OCC)cc(P(=O)(O)O)c2)o1)[C@@H](CC)N(C=O)OCc1oc(=O)oc1C]

To a rt solution containing dibenzyl (3-ethoxy-5-(5-((((R)-2-((R)-1-(N-((5-methyl-2-oxo-1,3-dioxol-4-yl)methoxy)formamido)propyl)heptanamido)methyl)carbamoyl)furan-2-yl)phenyl)phosphonate (70 mg, 0.083 mmol) in DCM (1 mL) was added TMS-Br (0.037 mL, 0.287 mmol). The reaction mixture was stirred for 1 h. The mixture was concentrated and purified by HPLC. Fractions were combined and lyophilized to afford the title compound as a colorless solid. (7.7 mg, 7% yield).

### Example 142

### (S)-dimethyl 2-(4-(5-((((R)-2-((R)-1-(N-hydroxyformamido)propyl)heptanamido)methyl)carbamoyl)furan-2-yl)-2-(2-methoxy-2-oxoethoxy)benzamido)succinate

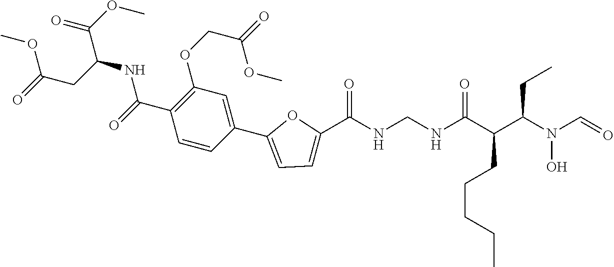 [SMILES: CCCCC[C@@H](C(=O)NCNC(=O)c1ccc(-c2ccc(C(=O)N[C@@H](CC(=O)OC)C(=O)OC)c(OCC(=O)OC)c2)o1)[C@@H](CC)N(O)C=O]

(S)-5-(4-((1,4-dimethoxy-1,4-dioxobutan-2-yl)carbamoyl)-3-(2-methoxy-2-oxoethoxy)phenyl)furan-2-carboxylic acid (55.62 g, 113 mmol) was taken up in CH₃CN (280 mL) and the resulting RT solution was treated with DIPEA (59.1 mL, 338 mmol). HATU (51.5 g, 135 mmol) was added followed by (R)—N-(aminomethyl)-2-((R)-1-(N-hydroxyformamido)propyl)heptanamide (31.5 g, 121 mmol) in DMF (280 mL). The resulting mixture was stirred at RT for 90 min. EtOAc (500 mL) was added followed by brine (500 mL). The layers were separated and the organics were washed with brine (3×). The combined brine washes were extracted with EtOAc (500 mL×2). The combined organics were dried over MgSO₄, filtered, and concentrated. Multiple purifications on Si (0-100% EtOAc/Hex followed by 10% MeOH/EtOAc) afforded the title compound as a brown gum. (19.5 g, 25% yield)

Example 143 were prepared from the indicated intermediate by methods analogous to those described for Example 142 from the Intermediates listed in the table below.

| Ex. | Name | Structure | Intermediate |
|---|---|---|---|
| 143 | (S)-diethyl 2-(2- ethoxy-4-(5-((((R)- 2-((R)-1-(N- hydroxyformamido) propyl)heptanamido) methyl)carbamoyl) furan-2-yl) benzamido) succinate | 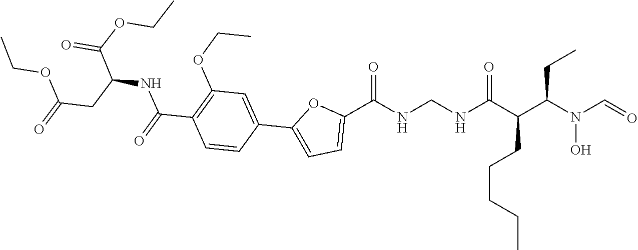 [SMILES: CCCCC[C@@H](C(=O)NCNC(=O)c1ccc(-c2ccc(C(=O)N[C@@H](CC(=O)OCC)C(=O)OCC)c(OCC)c2)o1)[C@@H](CC)N(O)C=O] | (S)-5-(4-((1,4- diethoxy-1,4- dioxobutan-2- yl)carbamoyl)- 3- ethoxyphenyl) furan-2- carboxylic acid |

### Example 200

### (4R,5R)-4-ethyl-3-formyl-6,10-dioxo-5-pentyl-10-(5-phenylfuran-2-yl)-2-oxa-3,7,9-triazadecyl Dihydrogen Phosphate, 2Ammonia Salt

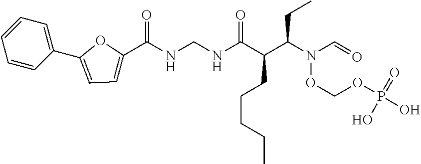 [SMILES: CCCCC[C@@H](C(=O)NCNC(=O)c1ccc(-c2ccccc2)o1)[C@@H](CC)N(C=O)OCOP(=O)(O)O]

TFA (1064 μl, 13.81 mmol) was added to a stirring mixture of di-tert-butyl ((4R,5R)-4-ethyl-3-formyl-6,10-dioxo-5-pentyl-10-(5-phenylfuran-2-yl)-2-oxa-3,7,9-triazadecyl) phosphate (300 mg, 0.460 mmol) in DCM (1534 μl). The resulting solution was stirred for 18 h and concentrated. Purification of the crude reaction mixture by reverse phase basic HPLC afforded the title compound as a colorless solid. (21 mg, 8% yield). MS (m/z) 540.5 (M+H)⁺

Examples 201-209 were prepared from the indicated intermediate by methods analogous to those described for Example 200 from the Intermediates listed in the table below.

| Ex. | Name | Structure | Intermediate |
|---|---|---|---|
| 201 | (S)-2-(2-ethoxy-4-(5- ((((R)-2-((R)-1-(N- ((2-methyl-6- (phosphonooxy) benzoyl)oxy) formamido) propyl)heptanamido) methyl)carbamoyl) furan-2-yl) benzamido) succinic acid | 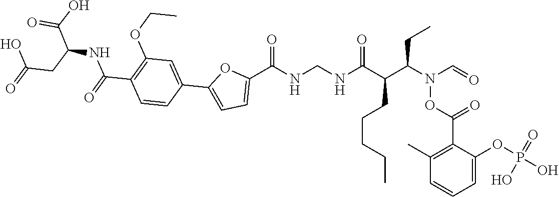 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1c(C)cccc1OP(=O)(O)O] | (S)-di-tert-butyl 2- (4-(5-((((R)-2-((R)- 1-(N-((2-((di-tert- butoxyphosphoryl) oxy)-6- methylbenzoyl)oxy) formamido)propyl) heptanamido)methyl) carbamoyl)furan-2- yl)-2-ethoxybenzamido) succinate |
| 202 | 4-(5-((((R)-2-((R)-1- (N-(benzoyloxy) formamido)propyl) heptanamido)methyl) carbamoyl)furan- 2-yl)benzoic acid | 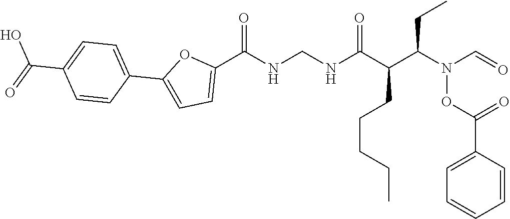 [SMILES: CCCCC[C@@H](C(=O)NCNC(=O)c1ccc(-c2ccc(C(=O)O)cc2)o1)[C@@H](CC)N(C=O)OC(=O)c1ccccc1] | tert-butyl 4-(5-((((R)- 2-((R)-1-(N- (benzoyloxy)formamido) propyl)heptanamido) methyl)carbamoyl)furan- 2-yl)benzoate |
| 203 | (S)-2-(2-ethoxy-4-(5- ((((R)-2-((R)-1-(N- ((phenylcarbamoyl) oxy)formamido) propyl)heptanamido) methyl)carbamoyl) furan-2-yl) benzamido) succinic acid | 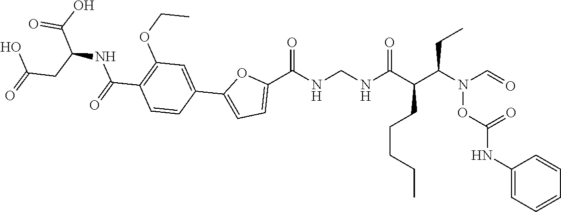 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)Nc1ccccc1] | (S)-di-tert-butyl 2- (2-ethoxy-4-(5-((((R)- 2-((R)-1-(N- ((phenylcarbamoyl)oxy) formamido)propyl) heptanamido)methyl) carbamoyl)furan-2- yl)benzamido)succinate |
| 204 | (S)-3-(2-ethoxy-4-(5- ((((R)-2-((R)-1-(N- hydroxyformamido) propyl)heptanamido) methyl)carbamoyl) furan-2-yl) benzamido)- 4-((5-methyl-2-oxo- 1,3-dioxol-4-yl) methoxy)-4- oxobutanoic acid | 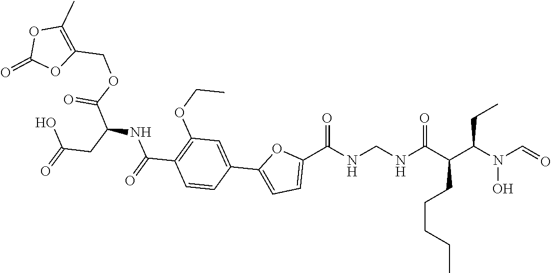 [SMILES: CCCCC[C@@H](C(=O)NCNC(=O)c1ccc(-c2ccc(C(=O)N[C@@H](CC(=O)O)C(=O)OCc3oc(=O)oc3C)c(OCC)c2)o1)[C@@H](CC)N(O)C=O] | (S)-4-tert-butyl 1-((5- methyl-2-oxo-1,3- dioxol-4-yl)methyl) 2- (2-ethoxy-4-(5-((((R)- 2-((R)-1-(N-((4- methoxybenzyl)oxy) formamido)propyl) heptanamido)methyl) carbamoyl)furan-2- yl)benzamido)succinate |
| 205 | (S)-2-(4-(5-((((R)-2- ((R)-1-(N-((2-bromo- 4-methoxybenzoyl) oxy)formamido) propyl)heptanamido) methyl)carbamoyl) furan-2-yl)- 2-ethoxybenzamido) succinic acid | 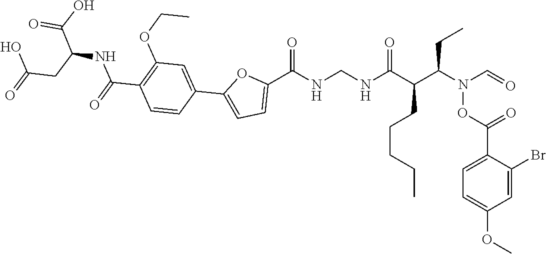 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1Br] | (S)-di-tert-butyl 2-(4-(5- ((((R)-2-((R)-1-(N-((2- bromo-4- methoxybenzoyl)oxy) formamido)propyl) heptanamido)methyl) carbamoyl)furan-2-yl)- 2-ethoxybenzamido) succinate |
| 206 | (S)-2-(4-(5-((((R)-2- ((R)-1-(N-((2-chloro- 4-methoxybenzoyl) oxy)formamido) propyl)heptanamido) methyl)carbamoyl) furan-2-yl)-2- ethoxybenzamido) succinic acid | 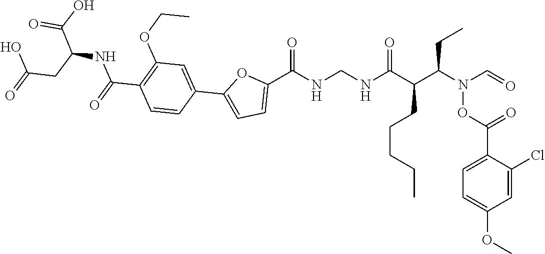 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(OC)cc1Cl] | (S)-di-tert-butyl 2- (4-(5-((((R)-2-((R)-1- (N-((2-chloro-4- methoxybenzoyl)oxy) formamido)propyl) heptanamido)methyl) carbamoyl)furan-2-yl)- 2-ethoxybenzamido) succinate |
| 207 | (S)-2-(2-ethoxy-4-(5- ((((R)-2-((R)-1-(N- ((4-(methylamino) benzoyl)oxy) formamido)propyl) heptanamido)methyl) carbamoyl)furan-2- yl)benzamido) succinic acid | 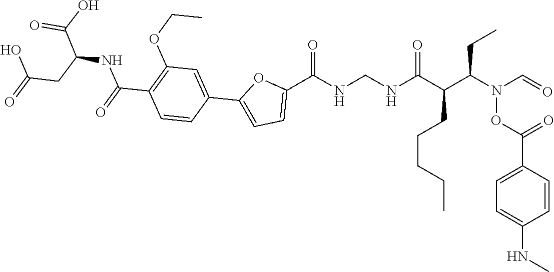 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(NC)cc1] | (S)-di-tert-butyl 2-(4- (5-((((R)-2-((R)-1-(N- ((4-((tert- butoxycarbonyl) (methyl)amino)benzoyl) oxy)formamido)propyl) heptanamido)methyl) carbamoyl)furan-2-yl)- 2-ethoxybenzamido) succinate |
| 208 | (S)-2-(4-(5-((((R)-2- ((R)-1-(N-((4- aminobenzoyl) oxy)formamido) propyl)heptanamido) methyl)carbamoyl) furan-2-yl)-2- ethoxybenzamido) succinic acid | 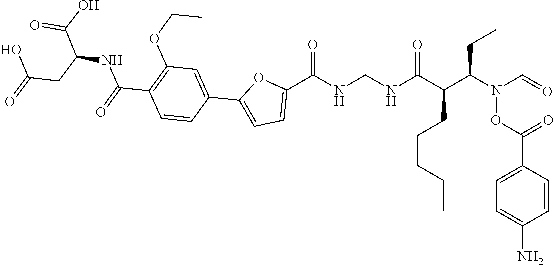 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(N)cc1] | (S)-di-tert-butyl 2-(4- (5-((((R)-2-((R)-1-(N- ((4-((tert- butoxycarbonyl) amino)benzoyl)oxy) formamido)propyl) heptanamido)methyl) carbamoyl)furan-2-yl)- 2-ethoxybenzamido) succinate |
| 209 | (S)-2-(4-(5-((((R)-2- ((R)-1-(N-((4- (aminomethyl) benzoyl)oxy) formamido)propyl) heptanamido)methyl) carbamoyl)furan- 2-yl)-2- ethoxybenzamido) succinic acid | 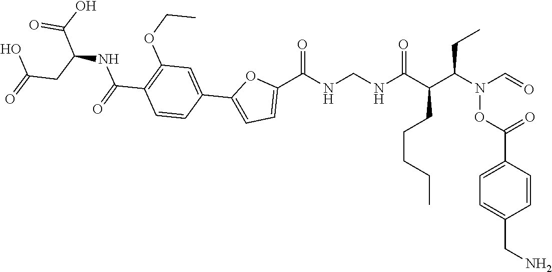 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc(CN)cc1] | (S)-di-tert-butyl 2-(4-(5- ((((R)-2-((R)-1-(N-((4- (((tert-butoxycarbonyl) amino)methyl)benzoyl) oxy)formamido)propyl) heptanamido)methyl) carbamoyl)furan-2-yl)- 2-ethoxybenzamido) succinate |

### Example 300

### (((3-ethoxy-5-(5-((((R)-2-((R)-1-(N-hydroxyformamido)propyl)heptanamido)methyl)carbamoyl)furan-2-yl)phenyl)(hydroxy)phosphoryl)oxy)methyl Isobutyrate

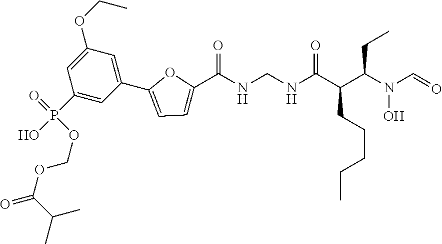 [SMILES: CCCCC[C@@H](C(=O)NCNC(=O)c1ccc(-c2cc(OCC)cc(P(=O)(O)OCOC(=O)C(C)C)c2)o1)[C@@H](CC)N(O)C=O]

A solution containing (3-ethoxy-5-(5-((((R)-2-((R)-1-(N-hydroxyformamido)propyl)heptanamido)methyl)carbamoyl)furan-2-yl)phenyl)phosphonic acid (0.1 g, 0.181 mmol), chloromethyl isopropyl carbonate (0.053 mL, 0.397 mmol), TEA (0.101 mL, 0.723 mmol), and sodium iodide (0.014 g, 0.090 mmol) in DMPU (1 mL) was stirred at 60° C. overnight. DMSO (1 mL) was added and the reaction was filtered through a syringe filter. Purification by reverse phase HPLC afforded the title compound as a colorless solid. (22 mg, 18% yield). MS (m/z) 654.3 (M+H)⁺

Examples 301-302 were prepared from the indicated intermediate by methods analogous to those described for Example 300 from the Intermediates listed in the table below.

| Ex. | Name | Structure | Intermediate |
|---|---|---|---|
| 301 | (5-methyl-2-oxo-1,3- dioxol-4-yl)methyl hydrogen (3-ethoxy- 5-(5-((((R)-2-((R)-1- (N- hydroxyformamido) propyl)heptanamido) methyl)carbamoyl) furan-2-yl)phenyl) phosphonate | 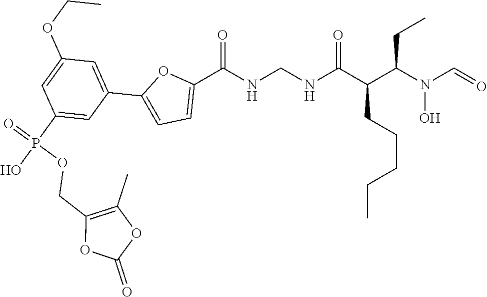 [SMILES: CCCCC[C@@H](C(=O)NCNC(=O)c1ccc(-c2cc(OCC)cc(P(=O)(O)OCc3oc(=O)oc3C)c2)o1)[C@@H](CC)N(O)C=O] | (3-ethoxy-5-(5- ((((R)-2-((R)-1-(N- hydroxyformamido) propyl)heptanamido) methyl)carbamoyl) furan-2-yl)phenyl) phosphonic acid |
| 302 | (((3-ethoxy-5-(5- ((((R)-2-((R)-1-(N- hydroxyformamido) propyl)heptanamido) methyl)carbamoyl) furan-2-yl)phenyl) (hydroxy)phosphoryl) oxy)methyl isopropyl carbonate | 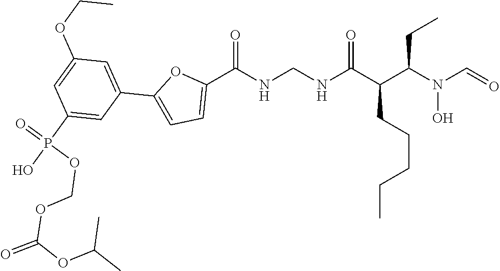 [SMILES: CCCCC[C@@H](C(=O)NCNC(=O)c1ccc(-c2cc(OCC)cc(P(=O)(O)OCOC(=O)OC(C)C)c2)o1)[C@@H](CC)N(O)C=O] | (3-ethoxy-5-(5- ((((R)-2-((R)-1-(N- hydroxyformamido) propyl)heptanamido) methyl)carbamoyl) furan-2-yl)phenyl) phosphonic acid |

### Example 400

### Phenyl Hydrogen (3-ethoxy-5-(5-((((R)-2-((R)-1-(N-hydroxyformamido)propyl)heptanamido)methyl)carbamoyl)furan-2-yl)phenyl)phosphonate, Ammonia Salt

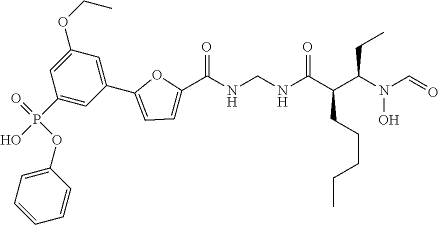 [SMILES: CCCCC[C@@H](C(=O)NCNC(=O)c1ccc(-c2cc(OCC)cc(P(=O)(O)Oc3ccccc3)c2)o1)[C@@H](CC)N(O)C=O]

To a solution containing diphenyl (3-ethoxy-5-(5-((((R)-2-((R)-1-(N-hydroxyformamido)propyl)heptanamido)methyl)carbamoyl)furan-2-yl)phenyl)phosphonate (0.190 g, 0.269 mmol) in THF (2.69 mL) was added sodium hydroxide (1M) (0.673 mL, 0.673 mmol). The reaction was stirred at RT for 5 h. The solvent was removed and the residue was diluted with water and extracted with EtOAc to remove remained SM. The water layer was acidified with 6N HCl until pH-2. The reaction was extracted with EtOAc, dried over Na2SO4 and concentrated. Purification by reverse phase HPLC afforded the title compound as a colorless solid. (85 mg, 49% yield). MS (m/z) 630.3 (M+H)⁺

### Example 500

### (S)-2-(2-ethoxy-4-(5-((((R)-2-((R)-1-(N-((2-methyl-4-nitrobenzoyl)oxy)formamido)propyl)heptanamido)methyl)carbamoyl)furan-2-yl)benzamido)succinic Acid

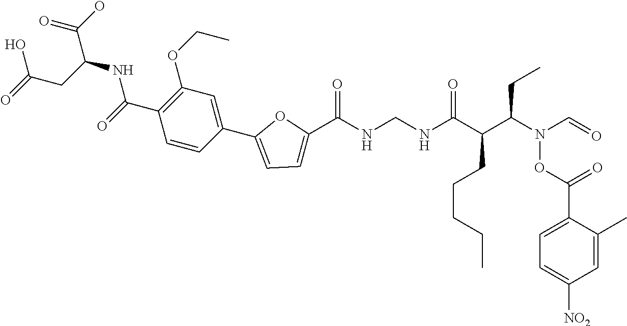 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1ccc([N+](=O)[O-])cc1C]

To a solution containing (S)-2-(2-ethoxy-4-(5-((((R)-2-((R)-1-(N-((2-methyl-4-nitrosobenzoyl)oxy)formamido)propyl)heptanamido)methyl)carbamoyl)furan-2-yl)benzamido)succinic acid (36.0 mg, 0.046 mmol) and (S)-2-(4-(5-((((R)-2-((R)-1-(N-((4-amino-2-methylbenzoyl)oxy)formamido)propyl)heptanamido)methyl)carbamoyl)furan-2-yl)-2-ethoxybenzamido)succinic acid (9.93 mg, 0.013 mmol) in acetic acid (1 mL) was added sodium perborate tetrahydrate (56.8 mg, 0.369 mmol). The vial was heated to 55° C. for 90 min. Purification by reverse phase Si-C18 (0-100% water with 0.1% TFA/MeCN) afforded the title compound. (16 mg, 23% yield). MS (m/z) 796.7 (M+H)⁺

### Example 600

### (S)-2-(4-(5-((((R)-2-((R)-1-(N-((3-aminobenzoyl)oxy)formamido)propyl)heptanamido)methyl)carbamoyl)furan-2-yl)-2-ethoxybenzamido)succinic Acid

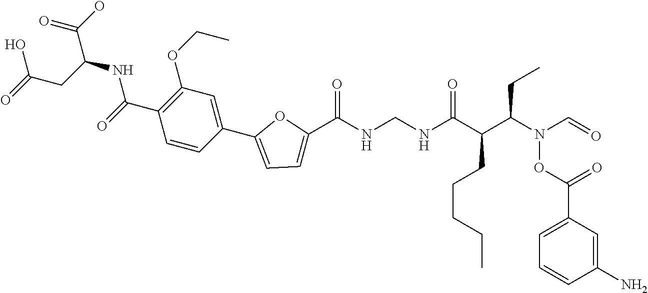 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1cccc(N)c1]

To a solution containing 3-((tert-butoxycarbonyl)amino)benzoic acid (35 mg, 0.15 mmol) in MeCN (1.2 mL) was added HATU (69.7 mg, 0.183 mmol) and DIPEA (32 μL, 0.183 mmol). The reaction was stirred for 30 min and (S)-di-tert-butyl 2-(2-ethoxy-4-(5-((((R)-2-((R)-1-(N-hydroxyformamido)propyl)heptanamido)methyl)carbamoyl)furan-2-yl)benzamido)succinate (91 mg, 0.122 mmol) in DMF (1 mL) was added. The reaction was stirred for 18 h at RT. The reaction was concentrated in-vacuo and the resulting residue was dissolved in DCM (3 mL) and treated with TFA (1 mL). The reactions stirred for 1 h at RT and additional TFA (1 mL) was added and stirred for 18 h at RT and 1 h at 50° C. The reaction was concentrated. Purification by reverse phase HPLC afforded the title compound as a colorless solid. (3 mg, 3% yield). MS (m/z) 752.6 (M+H)⁺

Examples 601-602 was prepared from the indicated intermediate by methods analogous to those described for Example 600 from the Intermediates listed in the table below.

| Ex. | Name | Structure | Intermediate |
|---|---|---|---|
| 601 | (S)-2-(4-(5-((((R)-2- ((R)-1-(N-((3- (dimethylamino) benzoyl)oxy) formamido)propyl) heptanamido)methyl) carbamoyl)furan-2- yl)-2- ethoxybenzamido) succinic acid | 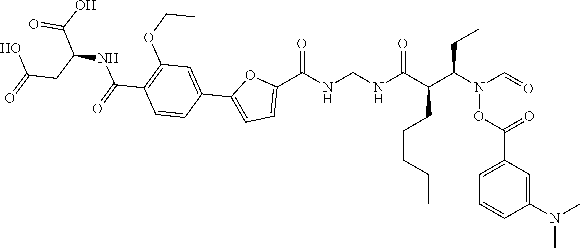 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1cccc(N(C)C)c1] | (S)-di-tert-butyl 2- (2-ethoxy-4-(5- ((((R)-2-((R)-1-(N- hydroxyformamido) propyl)heptanamido) methyl)carbamoyl) furan-2-yl) benzamido)succinate |
| 602 | (S)-2-(2-ethoxy-4-(5- ((((R)-2-((R)-1-(N- ((3-(methylamino) benzoyl)oxy) formamido)propyl) heptanamido)methyl) carbamoyl)furan-2- yl)benzamido) succinic acid | 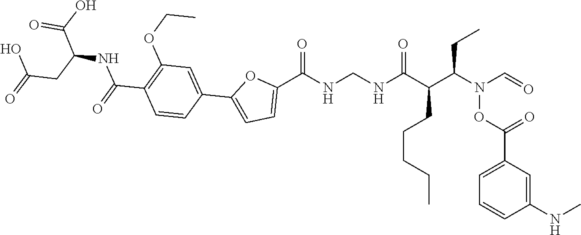 [SMILES: CCCCC[C@@H](C(=O)NCNC(=O)c1ccc(-c2ccc(C(=O)N[C@@H](CC(=O)O)C(=O)O)c(OCC)c2)o1)[C@@H](CC)N(C=O)OC(=O)c1cccc(NC)c1] | (S)-di-tert-butyl 2- (2-ethoxy-4-(5- ((((R)-2-((R)-1-(N- hydroxyformamido) propyl)heptanamido) methyl)carbamoyl) furan-2-yl) benzamido)succinate |

Tabulated spectroscopic data for Examples 1-14, 16-143, 200-209, 300-302, 400, 500, and 600-602:

| | | tR | |
|---|---|---|---|
| Ex. | ¹H NMR | (min) | MS (m/z) |
| 1 | ¹H NMR (DMSO-d₆) (Rotamers present in NMR) d: 12.96 (br. s, | 7.70^{a} | 737.3 |
| | 1H), 12.54 (br. s, 1H), 8.88 (d, J = 7.5 Hz, 2H), 8.14 (s, 1H), | | (M + H)⁺ |
| | 7.91-8.07 (m, 3H), 7.65-7.73 (m, 1H), 7.46-7.63 (m, 5H), 7.19-7.28 (m, | | |
| | 2H), 4.76-4.87 (m, 1H), 4.50-4.74 (m, 2H), 4.29 (q, J = 7.1 Hz, 2H), | | |
| | 3.96-4.05 (m, 1H), 2.70-2.96 (m, 3H), 1.65-1.76 (m, 1H), 1.47 (t, | | |
| | J = 6.9 Hz, 3H), 1.36-1.58 (m, 2H), 1.09-1.28 (m, 7H), 0.96 (t, J = 7.2 Hz, | | |
| | 3H), 0.73 (t, J = 6.9 Hz, 3H). | | |
| 2 | ¹H NMR (DMSO-d₆) (Rotamers present in NMR) d: 11.88 (br. s, | 6.89^{a} | 658.4 |
| | 2H), 9.15-9.20 (m, 0.3H), 9.07 (t, J = 4.6 Hz, 1H), 8.70-8.93 (m, | | (M + H)⁺ |
| | 0.7H), 8.12 (br. s, 1H), 8.06 (d, J = 5.0 Hz, 2H), 7.60-7.79 (m, 2H), | | |
| | 7.43-7.59 (m, 3H), 7.07-7.25 (m, 2H), 6.99 (br. s, 1H), 4.36-4.80 (m, | | |
| | 2H), 4.00 (m, 3H), 2.75 (m, 1H), 1.69 (m, 1H), 1.37-1.55 (m, 3H), | | |
| | 1.30 (t, J = 6.7 Hz, 3H), 1.06-1.25 (m, 6H), 0.96 (t, J = 6.9 Hz, 3H), | | |
| | 0.70 (t, J = 6.8 Hz, 3H). | | |
| 3 | ¹H NMR (DMSO-d₆) (Rotamers present in NMR) d: 10.70-12.77 (m, | 6.96^{a} | 672.3 |
| | 2H), 8.81-9.27 (m, 1H), 8.42-8.74 (m, 1H), 8.04 (br. s, 0.4H), | | (M + H)⁺ |
| | 7.57-7.78 (m, 1H), 7.50 (br. s, 0.6H), 7.33 (br. s, 8H), 7.03 (br. s, 1H), | | |
| | 4.41-4.74 (m, 2H), 3.94-4.07 (m, 2H), 3.77-3.91 (m, 2H), | | |
| | 3.52-3.61 (m, 1H), 2.53-2.65 (m, 1H), 1.36-1.67 (m, 3H), 1.27-1.34 (m, 3H), | | |
| | 1.02-1.26 (m, 7H), 0.62-0.89 (m, 6H). | | |
| 4 | ¹H NMR (DMSO-d₆) (Rotamers present in NMR) d: 12.33-13.23 (m, | 7.00^{a} | 751.3 |
| | 2H), 8.92-9.08 (m, 1H), 8.87 (d, J = 7.5 Hz, 1H), 8.46-8.72 (m, 1H), | | (M + H)⁺ |
| | 8.06 (s, 1H), 8.00 (d, J = 8.0 Hz, 1H), 7.63 (dd, J = 8.2, 1.4 Hz, 1H), | | |
| | 7.59-7.61 (m, 1H), 7.21-7.36 (m, 7H), 4.79-4.85 (m, 1H), | | |
| | 4.61-4.74 (m, 1H), 4.49-4.60 (m, 1H), 4.29 (q, J = 6.9 Hz, 2H), 3.77-3.92 (m, | | |
| | 1H), 3.33 (behind H₂O, s, 2H), 2.75-2.96 (m, 2H), 2.59 (m, 1H), | | |
| | 1.60 (m, 1H), 1.47 (t, J = 6.9 Hz, 3H), 1.32-1.44 (m, 3H), 1.09-1.29 (m, | | |
| | 6H), 0.78-0.90 (m, 3H), 0.73 (t, J = 6.9 Hz, 3H). | | |
| 5 | ¹H NMR (DMSO-d₆) (Rotamers present in NMR) d: 12.01-13.45 (m, | 6.96^{a} | 717.4 |
| | 2H), 8.88 (d, J = 7.8 Hz, 1H), 8.63-8.65 (m, 2H), 7.96-8.05 (m, 2H), | | (M + H)⁺ |
| | 7.64 (d, J = 1.3 Hz, 1H), 7.62 (s, 1H), 7.28 (dd, J = 18.4, 3.6 Hz, 2H), | | |
| | 4.82 (dt, J = 7.8, 4.9 Hz, 1H), 4.68 (dt, J = 12.8, 6.1 Hz, 1H), | | |
| | 4.50-4.60 (m, 1H), 4.30 (q, J = 6.9 Hz, 2H), 3.86 (br. s, 1H), 2.75-2.97 (m, 2H), | | |
| | 2.55 (br. s, 1H), 1.59-1.72 (m, 1H), 1.47 (t, J = 6.9 Hz, 3H), | | |
| | 1.24-1.28 (m, 5H), 1.18 (s, 9H), 1.09-1.15 (m, 4H), 0.89 (t, J = 7.2 Hz, 3H), | | |
| | 0.73 (t, J = 6.9 Hz, 3H). | | |
| 6 | ¹H NMR (DMSO-d₆) (Rotamers present in NMR) d: 12.39-13.74 (m, | 0.96^{e} | 783.5 |
| | 3H), 10.11 (s, 1H), 9.12 (d, J = 7.8 Hz, 1H), 8.98 (m, 1H), 8.74 (m, | | (M + H)⁺ |
| | 1H), 8.35 (s, 0.2H), 8.15 (s, 0.8H), 7.98 (d, J = 8.3 Hz, 1H), 7.88 (m, | | |
| | 1H), 7.65 (dd, J = 8.2, 1.4 Hz, 1H), 7.57 (d, J = 1.3 Hz, 1H), | | |
| | 7.48-7.55 (m, J = 15.6, 1.5 Hz, 1H), 7.21-7.31 (m, 2H), 6.99 (d, J = 8.0 Hz, 1H), | | |
| | 6.92 (t, J = 7.4 Hz, 1H), 4.97 (s, 2H), 4.76-4.84 (m, 1H), | | |
| | 4.65-4.74 (m, 0.8H), 4.56 (dd, J = 12.0, 6.0 Hz, 1H), 4.08-4.15 (m, 0.2H), | | |
| | 3.18 (br. s, 1H), 2.73-2.90 (m, 2H), 2.68 (m, 1H), 1.70 (m, 1H), 1.45 (m, | | |
| | 3H), 1.05-1.27 (m, 6H), 0.95 (t, J = 7.0 Hz, 3H), 0.71 (t, J = 6.9 Hz, | | |
| | 3H). | | |
| 7 | ¹H NMR (DMSO-d₆) (Rotamers present in NMR) d: 12.19-13.68 (m, | 2.59^{b} | 767.3 |
| | 3H), 9.13 (d, J = 7.0 Hz, 1H), 8.76-9.00 (m, 1H), 8.34-8.37 (s, 0.1H), | | (M + H)⁺ |
| | 8.14 (s., 0.9H), 7.91-8.08 (m, 3H), 7.58-7.74 (m, J = 19.7, 7.2 Hz, | | |
| | 2H), 7.46-7.58 (m, J = 13.6 Hz, 3H), 7.23 (br. s, 2H), 4.96 (s., 2H), | | |
| | 4.76-4.85 (m, 1H), 4.65-4.75 (m, 1H), 4.51-4.63 (m, 1H), | | |
| | 4.06-4.18 (m, 1H), 3.93-4.06 (m, 1H), 2.61-2.90 (m, 3H), 1.59-1.78 (m, 1H), | | |
| | 1.32-1.57 (m, 3H), 1.03-1.30 (m, 6H), 0.90-1.02 (m, 3H), | | |
| | 0.62-0.78 (m, 3H). | | |
| 8 | ¹H NMR (DMSO-d₆) (Rotamers present in NMR) d: 11.87-14.21 (m, | 2.66^{b} | 781.3 |
| | 3H), 9.12 (d, J = 8.0 Hz, 1H), 8.88-9.02 (m, 1H), 8.64-8.84 (m, 0.9H), | | (M + H)⁺ |
| | 8.58-8.63 (m, 0.1H), 8.37 (s, 0.1H), 8.15 (s, 0.9H), 7.98 (d, J = 8.3 Hz, | | |
| | 1H), 7.90-7.96 (m, 1H), 7.65 (dd, J = 8.3, 1.3 Hz, 1H), 7.57 (s, | | |
| | 1H), 7.49-7.55 (m, 1H), 7.29-7.43 (m, 2H), 7.25 (dd, J = 1.0 Hz, 2H), | | |
| | 4.97 (s, 2H), 4.81 (q, J = 1.0 Hz, 1H), 4.65-4.75 (m, 1H), | | |
| | 4.51-4.61 (m, 1H), 3.96-4.18 (1H), 2.73-2.90 (m, 2H), 2.60-2.72 (m, 1H), | | |
| | 2.45 (s, 3H), 1.62-1.78 (m, 1H), 1.35-1.58 (m, 3H), 1.07-1.26 (m, 6H), | | |
| | 0.96 (t, J = 6.5 Hz, 3H), 0.71 (t, J = 6.9 Hz, 3H). | | |
| 9 | ¹H NMR (MEOH-d₄) (Rotamers present in NMR) d: 8.81 (t, J = 5.8 Hz, | 7.70^{j} | 668.2 |
| | 1H), 8.73-8.78 (m, 1H), 8.33 (s, 0.4H), 8.02 (s, 0.6H), | | (M + H)⁺ |
| | 7.75-7.85 (m, 1H), 7.66 (br. s, 1H), 7.31 (dd, J = 15.1, 1.5 Hz, 1H), 7.27 (t, | | |
| | J = 3.5 Hz, 1H), 7.02 (d, J = 3.5 Hz, 1H), 5.49-5.63 (m, 1H), | | |
| | 5.36-5.49 (m, 1H), 4.68-4.85 (m, 2H), 4.18 (q, J = 7.0 Hz, 2H), 3.68-3.78 (m, | | |
| | 1H), 2.58-2.80 (m, 1H), 1.61-1.86 (m, 1H), 1.51-1.60 (m, 2H), | | |
| | 1.46 (t, J = 6.9 Hz, 3H), 1.22-1.37 (m, 4H), 1.20 (s, 9H), 1.13-1.16 (m, 3H), | | |
| | 0.96-1.04 (m, 1.8H), 0.88-0.95 (m, 1.2H), 0.74-0.85 (m, 3H). | | |
| 10 | ¹H NMR (DMSO-d₆) (Rotamers present in NMR) d: 9.11-9.22 (m, | 1.56^{d} | 664.1 |
| | 0.6H), 9.02 (t, J = 5.4 Hz, 0.4H), 8.53 (s, 0.6H), 8.41-8.52 (m, 1H), | | (M + H)⁺ |
| | 7.94 (s, 0.4H), 7.65-7.82 (m, 1H), 7.56 (s, 1H), 7.20-7.32 (m, 1H), | | |
| | 7.17 (d, J = 3.5 Hz, 2H), 5.14-5.40 (m, 2H), 4.51-4.78 (m, 2H), | | |
| | 4.16-4.24 (m, 0.6H), 4.12 (q, J = 6.9 Hz, 2H), 3.66-3.79 (m, 0.4H), | | |
| | 2.56-2.76 (m, 1H), 1.52-1.68 (m, 2H), 1.37 (s, 5H), 1.03-1.28 (m, 6H), | | |
| | 0.67-1.01 (m, 6H). | | |
| 11 | ¹H NMR (DMSO-d₆) (Rotamers present in NMR) d: 11.96-13.11 (m, | 0.66^{e} | 773.3 |
| | 3H), 9.83-9.93 (m, 0.5H), 9.51-9.59 (m, 0.5H), 9.27-9.40 (m, 1H), | | (M + H)⁺ |
| | 8.42-8.61 (m, 1H), 8.35 (s, 1H), 7.98 (d, J = 8.3 Hz, 2H), 7.92 (s, | | |
| | 0.5H), 7.83 (s, 0.5H), 7.58 (d, J = 8.0 Hz, 1H), 7.16-7.41 (m, 3H), | | |
| | 5.13-5.33 (m, 1H), 4.94-5.12 (m, 1H), 4.71-4.82 (m, 0.5H), | | |
| | 4.44-4.55 (m, 0.5H), 4.06-4.19 (m, 0.5H), 3.69-3.80 (m, 0.5H), | | |
| | 3.54-3.67 (m, 2H), 3.17 (s, 2H), 3.09-3.15 (m, 1H), 2.68-2.88 (m, 2H), | | |
| | 1.48-1.69 (m, 2H), 1.35-1.45 (m, 2H), 1.20-1.32 (m, 6H), 0.69-0.89 (m, | | |
| | 6H). | | |
| 12 | ¹H NMR (DMSO-d₆) (Rotamers present in NMR) d: 12.20-13.99 (m, | 2.55^{b} | 825.3 |
| | 3H), 9.12 (d, J = 7.8 Hz, 1H), 8.88-9.04 (m, 1H), 8.67-8.87 (m, 1H), | | (M + H)⁺ |
| | 8.03-8.20 (m, 2H), 7.99 (d, J = 8.0 Hz, 1H), 7.72 (td, J = 7.8, 1.6 Hz, | | |
| | 1H), 7.65 (dd, J = 8.2, 1.1 Hz, 1H), 7.56 (d, J = 1.3 Hz, 1H), 7.41 (t, | | |
| | J = 7.4 Hz, 1H), 7.23-7.32 (m, 3H), 4.97 (s, 2H), 4.76-4.86 (m, J = 6.3 Hz, | | |
| | 1H), 4.64-4.76 (m, 1H), 4.49-4.62 (m, 1H), 3.91-4.06 (m, 1H), | | |
| | 2.73-2.90 (m, 2H), 2.60-2.71 (m, 1H), 2.21 (s, 3H), 1.68 (m, 1H), | | |
| | 1.44 (m, 3H), 1.05-1.28 (m, 6H), 0.93 (t, J = 7.0 Hz, 3H), 0.72 (t, | | |
| | J = 6.9 Hz, 3H). | | |
| 13 | ¹H NMR (DMSO-d₆) (Rotamers present in NMR) d: 11.93-13.87 (m, | 5.80^{a} | 768.3 |
| | 3H), 9.18 (br. s, 1H), 9.07-9.14 (m, 1H), 9.03 (t, J = 6.1 Hz, 0.6H), | | (M + H)⁺ |
| | 8.84 (dd, J = 4.8, 1.5 Hz, 0.6H), 8.81 (dd, J = 4.9, 1.6 Hz, 0.4H), | | |
| | 8.54 (t, J = 6.0 Hz, 0.4H), 8.35 (t, J = 1.9 Hz, 0.4H), 8.33 (t, J = 1.9 Hz, | | |
| | 0.6H), 8.29-8.32 (m, 1H), 8.16 (br. s, 0.6H), 7.94-8.04 (m, 1H), | | |
| | 7.76 (s, 0.4H), 7.69 (dd, J = 8.2, 1.4 Hz, 0.6H), 7.49-7.65 (m, 2.4H), | | |
| | 7.25-7.34 (m, 1H), 7.20 (m, 1H), 4.97 (s, 2H), 4.78-4.86 (m, 1H), | | |
| | 4.50-4.76 (m, 2H), 3.97-4.08 (m, 0.6H), 3.52-3.62 (m, 0.4H), | | |
| | 2.69-2.91 (m, 2H), 2.55-2.65 (m, 1H), 1.62-1.74 (m, 1H), 1.31-1.59 (m, 3H), | | |
| | 1.05-1.29 (m, 6H), 0.87-1.02 (m, 2H), 0.68-0.82 (m, 4H). | | |
| 14 | ¹H NMR (DMSO-d₆) (Rotamers present in NMR) d: 12.27-13.80 (m, | 0.95^{e} | 763.7 |
| | 3H), 9.12 (d, J = 7.8 Hz, 1H), 8.97 (t, J = 5.6 Hz, 1H), 8.64 (br. s, 1H), | | (M + H)⁺ |
| | 7.96-8.06 (m, 2H), 7.68 (dd, J = 8.0, 1.3 Hz, 1H), 7.59 (d, J = 1.0 Hz, | | |
| | 1H), 7.12-7.34 (m, 2H), 4.93-5.04 (m, 2H), 4.76-4.86 (m, 1H), | | |
| | 4.46-4.74 (m, 2H), 3.91 (t, J = 9.2 Hz, 1H), 2.72-2.90 (m, 2H), | | |
| | 2.55-2.65 (m, 1H), 1.73-1.80 (m, 2H), 1.60-1.71 (m, 1H), 1.45-1.50 (m, 1H), | | |
| | 1.41 (s, 9H), 1.09-1.22 (m, 6H), 0.78-0.93 (m, 3H), 0.67-0.75 (m, | | |
| | 3H). | | |
| 16 | ¹H NMR (MEOH-d₄) (Rotamers present in NMR) d: 8.71-9.06 (m, | 2.72^{b} | 674.3 |
| | 1H), 8.20 (s, 1H), 7.89 (d, J = 6.8 Hz, 1H), 7.69-7.82 (m, 1H), | | (M + H)⁺ |
| | 7.55-7.63 (m, 1H), 7.39-7.50 (m, 1H), 7.24-7.34 (m, 1H), 7.20 (d, J = 3.8 Hz, | | |
| | 1H), 6.79-6.99 (m, 4H), 4.74-4.89 (m, 3H), 4.10-4.23 (m, 2H), | | |
| | 3.98-4.09 (m, 1H), 2.59-2.70 (m, 1H), 1.76-1.91 (m, 1H), | | |
| | 1.50-1.75 (m, 3H), 1.44 (t, J = 6.9 Hz, 3H), 1.18-1.38 (m, 6H), 1.01-1.13 (m, | | |
| | 3H), 0.76-0.89 (m, 3H). | | |
| 17 | ¹H NMR (DMSO-d₆) (Rotamers present in NMR) d: | 1.82^{b} | 811.2 |
| | 12.12-14.03 (br.s., 4H), 9.13 (d, J = 7.8 Hz, 1H), 8.67-9.01 (m, 2H), 8.41 (br. s, | | (M + H)⁺ |
| | 1H), 8.26-8.36 (m, 1H), 8.22 (d, J = 7.8 Hz, 1H), 8.18 (s, 1H), 7.98 (d, | | |
| | J = 8.0 Hz, 1H), 7.59-7.75 (m, 2H), 7.54 (s, 1H), 7.18-7.27 (m, 2H), | | |
| | 4.96 (s, 2H), 4.77-4.86 (m, 1H), 4.52-4.76 (m, 2H), 4.00-4.10 (br. s, | | |
| | 1H), 2.74-2.89 (m, 2H), 2.65-2.73 (m, 1H), 1.65-1.79 (m, 1H), | | |
| | 1.32-1.61 (m, 3H), 1.03-1.28 (m, 6H), 0.96 (t, J = 7.2 Hz, 3H), 0.71 (t, | | |
| | J = 6.9 Hz, 3H). | | |
| 18 | ¹H NMR (DMSO-d₆) (Rotamers present in NMR) d: 12.19-13.90 (m, | 2.53^{e} | 745.3 |
| | 3H), 9.12 (d, J = 7.8 Hz, 1H), 8.80-8.94 (m, 1H), 8.57-8.73 (m, 1H), | | (M + H)⁺ |
| | 7.96-8.03 (m, 2H), 7.68 (d, J = 8.3 Hz, 1H), 7.59 (s, 1H), 7.30 (dd, | | |
| | J = 16.6, 3.5 Hz, 2H), 4.97 (s, 2H), 4.81 (q, J = 6.5 Hz, 1H), | | |
| | 4.47-4.73 (m, 2H), 3.78-3.91 (m, 1H), 2.72-2.91 (m, 2H), 2.53-2.59 (m, 1H), | | |
| | 1.54-1.71 (m, 1H), 1.34-1.51 (m, J = 8.8 Hz, 2H), 1.23-1.29 (m, 2H), | | |
| | 1.22 (s, 3H), 1.08-1.20 (m, 6H), 0.82-0.91 (m, 3H), 0.68-0.81 (m, | | |
| | 6H). | | |
| 19 | ¹H NMR (DMSO-d₆) (Rotamers present in NMR) d: 12.24-13.79 (m, | 2.54^{e} | 745.3 |
| | 3H), 9.12 (d, J = 7.8 Hz, 1H), 8.85 (br. s, 1H), 8.67 (br. s, 1H), | | (M + H)⁺ |
| | 7.96-8.07 (m, 2H), 7.68 (d, J = 8.8 Hz, 1H), 7.59 (s, 1H), 7.26-7.34 (m, | | |
| | 2H), 4.97 (s, 2H), 4.75-4.85 (m, 1H), 4.47-4.74 (m, 2H), | | |
| | 3.79-3.93 (m, 1H), 2.72-2.90 (m, 2H), 2.53-2.64 (m, 1H), 1.58-1.69 (m, 1H), | | |
| | 1.50-1.57 (m, 1H), 1.36-1.50 (m, 2H), 1.27-1.35 (m, 2H), | | |
| | 1.09-1.26 (m, 6H), 1.05 (t, J = 5.5 Hz, 3H), 0.94-1.02 (m, 1H), 0.88 (t, J = 6.3 Hz, | | |
| | 3H), 0.78-0.84 (m, 1H), 0.73 (t, J = 6.5 Hz, 3H). | | |
| 20 | ¹H NMR (DMSO-d₆) (Rotamers present in NMR) d: 12.16-13.89 (m, | 2.60^{b} | 797.3 |
| | 3H), 9.11 (d, J = 7.8 Hz, 1H), 8.73-8.98 (m, 2H), 8.11 (s, 1H), | | (M + H)⁺ |
| | 7.86-8.03 (m, 3H), 7.62 (d, J = 7.8 Hz, 1H), 7.54 (s, 1H), 7.20-7.31 (m, | | |
| | 20 2H), 6.91-7.05 (m, 2H), 4.96 (s, 2H), 4.82 (q, J = 6.7 Hz, 1H), | | |
| | 4.50-4.76 (m, 2H), 3.90-4.03 (m, 1H), 3.82 (s, 3H), 2.74-2.91 (m, 2H), | | |
| | 2.65-2.73 (m, 1H), 1.60-1.75 (m, 1H), 1.40-1.58 (m, 2H), | | |
| | 1.05-1.33 (m, 7H), 0.96 (t, J = 6.9 Hz, 3H), 0.73 (t, J = 6.8 Hz, 3H). | | |
| 21 | ¹H NMR (DMSO-d₆) (Rotamers present in NMR) d: 12.34-13.89 (m, | 2.28^{b} | 720.2 |
| | 3H), 9.12 (d, J = 7.5 Hz, 1H), 8.97 (t, J = 5.5 Hz, 1H), 8.44 (t, J = 5.4 Hz, | | (M + H)⁺ |
| | 1H), 7.97-8.03 (m, 2H), 7.68 (d, J = 8.3 Hz, 1H), 7.59 (s, 1H), | | |
| | 7.36-7.47 (m, 1H), 7.31 (d, J = 3.5 Hz, 1H), 7.27 (d, J = 3.5 Hz, 1H), | | |
| | 4.98 (s, 2H), 4.81 (q, J = 7.0 Hz, 1H), 4.50-4.72 (m, 2H), | | |
| | 3.75-3.86 (m, 1H), 2.72-2.92 (m, 2H), 2.66 (d, J = 4.3 Hz, 1H), 2.58 (d, J = 4.5 Hz, | | |
| | 2H), 2.53-2.63 (m, 1H), 1.56-1.70 (m, 1H), 1.29-1.54 (m, 3H), | | |
| | 1.05-1.24 (m, 6H), 0.79-0.93 (m, 3H), 0.72 (t, J = 6.3 Hz, 3H). | | |
| 22 | ¹H NMR (DMSO-d₆) (Rotamers present in NMR) d: 12.25-13.80 (m, | 0.98^{e} | 782.7 |
| | 3H), 9.14 (d, J = 7.3 Hz, 1H), 8.97-9.07 (m, J = 1.0, 1.0 Hz, 1H), | | (M + H)⁺ |
| | 8.75-8.90 (m, 1H), 8.10 (s, 1H), 7.99 (d, J = 8.3 Hz, 1H), 7.80-7.90 (m, | | |
| | 1H), 7.66 (dd, J = 8.3, 1.3 Hz, 1H), 7.57 (d, J = 1.0 Hz, 1H), | | |
| | 7.22-7.34 (m, 3H), 6.74-6.81 (m, 1H), 6.62 (br. s, 2H), 6.48-6.57 (m, 1H), | | |
| | 4.97 (s, 2H), 4.80 (q, J = 6.7 Hz, 1H), 4.49-4.75 (m, 2H), 3.95-4.05 (m, | | |
| | 1H), 2.76-2.88 (m, 2H), 2.64-2.73 (m, 1H), 1.63-1.74 (m, 1H), | | |
| | 1.29-1.53 (m, 3H), 1.04-1.26 (m, 6H), 0.96 (t, J = 7.2 Hz, 3H), 0.71 (t, | | |
| | J = 7.0 Hz, 3H). | | |
| 23 | ¹H NMR (DMSO-d₆) (Rotamers present in NMR) d: 12.88 (br. s, | 1.04^{e} | 796.5 |
| | 3H), 9.14 (d, J = 7.0 Hz, 1H), 8.72-8.96 (m, 2H), 8.11 (s, 1H), 7.98 (s, | | (M + H)⁺ |
| | 1H), 7.88-7.95 (m, 1H), 7.62 (d, J = 8.0 Hz, 1H), 7.54 (s, 1H), 7.41 (t, | | |
| | J = 7.7 Hz, 1H), 7.20-7.27 (m, 3H), 6.65 (d, J = 9.0 Hz, 1H), 6.53 (t, | | |
| | J = 7.8 Hz, 1H), 4.95 (s, 2H), 4.76-4.84 (m, 1H), 4.51-4.77 (m, 2H), | | |
| | 3.91-4.04 (m, 1H), 2.78-2.88 (m, 2H), 2.75 (s, 3H), 2.64-2.71 (m, | | |
| | 1H), 1.59-1.74 (m, 1H), 1.29-1.53 (m, 3H), 1.07-1.27 (m, 6H), | | |
| | 0.96 (t, J = 7.0 Hz, 3H), 0.73 (t, J = 6.7 Hz, 3H). | | |
| 24 | ¹H NMR (DMSO-d₆) (Rotamers present in NMR) d: 12.19-13.75 (m, | 1.07^{e} | 761.5 |
| | 3H), 9.13 (d, J = 7.5 Hz, 1H), 8.80-8.90 (m, 1H), 8.62-8.78 (m, 1H), | | (M + H)⁺ |
| | 7.96-8.05 (m, 2H), 7.68 (dd, J = 8.2, 1.4 Hz, 1H), 7.60 (d, J = 1.0 Hz, | | |
| | 1H), 7.29 (dd, J = 15.9, 3.6 Hz, 2H), 4.97 (s, 2H), 4.75-4.85 (m, 1H), | | |
| | 4.49-4.73 (m, 2H), 3.80-3.92 (m, 1H), 2.72-2.91 (m, 2H), | | |
| | 2.55-2.60 (m, 1H), 2.30-2.41 (m, 1H), 1.61-1.77 (m, 1H), 1.38-1.59 (m, 6H), | | |
| | 1.07-1.28 (m, 7H), 0.91 (t, J = 6.8 Hz, 3H), 0.80-0.87 (m, 6H), 0.72 (t, | | |
| | J = 6.9 Hz, 3H). | | |
| 25 | ¹H NMR (DMSO-d₆) (Rotamers present in NMR) d: 13.51 (br. s, | 0.99^{k} | 786.4 |
| | 1H), 12.31-13.06 (m, 2H), 9.03-9.14 (m, 2H), 8.51-8.71 (m, 1H), | | (M + H)⁺ |
| | 8.17 (br. s, 1H), 7.99 (d, J = 8.3 Hz, 1H), 7.68 (dd, J = 8.3, 1.3 Hz, 1H), | | |
| | 7.58 (s, 1H), 7.28 (dd, J = 15.9, 3.6 Hz, 2H), 4.99 (s, 2H), | | |
| | 4.77-4.87 (m, 1H), 4.47-4.72 (m, 2H), 3.97-4.12 (m, 1H), 2.72-2.92 (m, 2H), | | |
| | 2.56-2.66 (m, 4H), 2.32 (s, 3H), 1.64-1.78 (m, 1H), 1.36-1.57 (m, | | |
| | 3H), 1.05-1.27 (m, 6H), 0.82-0.98 (m, 3H), 0.66-0.77 (m, 3H). | | |
| 26 | ¹H NMR (DMSO-d₆) (Rotamers present in NMR) d: 13.52 (br. s, | 0.87^{k} | 796.4 |
| | 1H), 12.19-13.11 (m, 2H), 9.10 (d, J = 7.8 Hz, 1H), 8.98 (br. s, 1H), | | (M + H)⁺ |
| | 8.70 (br. s, 1H), 8.45 (d, J = 5.3 Hz, 1H), 8.15 (s, 1H), 7.99 (d, J = 8.3 Hz, | | |
| | 1H), 7.68 (d, J = 9.5 Hz, 1H), 7.59 (s, 1H), 7.28 (dd, J = 22.4, 3.6 Hz, | | |
| | 2H), 7.23 (d, J = 5.0 Hz, 1H), 4.99 (s, 2H), 4.77-4.86 (m, 1H), | | |
| | 4.41-4.72 (m, 2H), 3.99-4.12 (m, 1H), 2.73-2.90 (m, 2H), | | |
| | 2.57-2.70 (m, 1H), 2.55 (s, 3H), 2.38 (s, 3H), 1.68-1.81 (m, 1H), 1.34-1.52 (m, | | |
| | 3H), 1.05-1.22 (m, 6H), 0.92-1.03 (m, 3H), 0.69 (t, J = 7.0 Hz, 3H). | | |
| 27 | ¹H NMR (DMSO-d₆) (Rotamers present in NMR) d: 13.51 (br. s, | 1.21^{l} | 857.4 |
| | 1H), 12.08-13.16 (m, 2H), 9.11 (d, J = 7.8 Hz, 1H), 8.97-9.06 (m, 1H), | | (M + H)⁺ |
| | 8.49-8.60 (m, 1H), 8.08 (s, 1H), 8.00 (d, J = 8.0 Hz, 1H), 7.68 (d, | | |
| | J = 9.0 Hz, 1H), 7.59 (s, 1H), 7.22-7.44 (m, 12H), 5.44 (s, 1H), | | |
| | 4.98 (s, 2H), 4.76-4.88 (m, 1H), 4.42-4.73 (m, 2H), 3.80-3.95 (m, 1H), | | |
| | 2.73-2.91 (m, 2H), 2.40-2.48 (m, 1H), 1.46-1.57 (m, J = 5.0 Hz, 1H), | | |
| | 1.34-1.45 (m, 1H), 1.18-1.34 (m, 1H), 0.92-1.16 (m, 7H), | | |
| | 0.74-0.84 (m, 3H), 0.70 (t, J = 6.7 Hz, 3H). | | |
| 28 | ¹H NMR (DMSO-d₆) (Rotamers present in NMR) d: 12.21-14.15 (m, | 1.18^{k} | 809.4 |
| | 3H), 9.13 (d, J = 6.0 Hz, 1H), 8.92-9.03 (m, 1H), 8.64-8.82 (m, 1H), | | (M + H)⁺ |
| | 8.10-8.21 (m, 1H), 7.98 (d, J = 8.3 Hz, 1H), 7.79 (d, J = 7.3 Hz, 1H), | | |
| | 7.67 (dd, J = 8.3, 1.3 Hz, 1H), 7.55-7.62 (m, 2H), 7.48-7.54 (m, 1H), | | |
| | 7.23-7.35 (m, 3H), 4.97 (s, 2H), 4.76-4.85 (m, J = 6.5 Hz, 1H), | | |
| | 4.49-4.75 (m, 2H), 3.97-4.15 (m, 1H), 3.49-3.63 (m, 1H), 2.73-2.90 (m, | | |
| | 2H), 2.61-2.72 (m, 1H), 1.63-1.81 (m, 1H), 1.35-1.58 (m, 3H), | | |
| | 1.05-1.28 (m, 12H), 0.90-1.02 (m, J = 6.5 Hz, 3H), 0.71 (t, J = 7.0 Hz, 3H). | | |
| 29 | ¹H NMR (DMSO-d₆) (Rotamers present in NMR) d: 13.50 (br. s, | 6.98^{a} | 663.1 |
| | 1H), 12.78 (br. s, 1H), 12.46 (br. s, 1H), 9.21 (t, J = 5.6 Hz, 1H), | | (M − tBuNCO + 1) |
| | 9.11 (d, J = 7.8 Hz, 0.3H), 8.92 (t, J = 5.9 Hz, 0.7H), 8.31-8.44 (m, 1H), | | |
| | 8.00-8.02 (m, 1H), 7.99 (s, 1H), 7.67 (dd, J = 8.3, 1.3 Hz, 1H), | | |
| | 7.59 (d, J = 1.0 Hz, 1H), 7.26-7.34 (m, 2H), 7.12-7.25 (m, 1H), | | |
| | 4.95-5.01 (m, 2H), 4.82 (q, J = 7.2 Hz, 1H), 4.48-4.73 (m4, 2H), 4.15-4.24 (m, | | |
| | 0.3H), 3.69-3.82 (m, 0.7H), 2.73-2.91 (m, 2H), 2.53-2.61 (m, 1H), | | |
| | 1.57-1.71 (m, 1H), 1.31-1.55 (m, 1H), 1.30-1.45 (m, 4H), 1.25 (s, | | |
| | 3H), 1.18 (s, 6H), 1.09-1.23 (m, 4H), 0.90 (t, J = 7.0 Hz, 2H), 0.83 (t, | | |
| | J = 7.2 Hz, 1H), 0.72 (t, J = 6.7 Hz, 3H). | | |
| 30 | ¹H NMR (DMSO-d₆) (Rotamers present in NMR) d: 12.17-13.88 (m, | 6.56^{a} | 748.2 |
| | 3H), 9.13-9.19 (m, 0.2H), 9.12 (d, J = 7.8 Hz, 1H), 8.93 (t, J = 5.1 Hz, | | (M + H)⁺ |
| | 0.8H), 8.40 (t, J = 5.1 Hz, 0.8H), 8.34 (t, J = 5.0 Hz, 0.2H), 8.12 (s, | | |
| | 0.2H), 8.01 (s, 1H), 7.99 (s, 0.8H), 7.68 (d, J = 8.3 Hz, 1H), 7.59 (s, | | |
| | 1H), 7.34-7.46 (m, 1H), 7.26-7.33 (m, 2H), 4.98 (br. s, 2H), | | |
| | 4.77-4.86 (m, 1H), 4.49-4.76 (m, 2H), 4.18-4.28 (m, 0.2H), 3.74-3.86 (m, | | |
| | 0.8H), 2.73-2.92 (m, 2H), 2.53-2.61 (m, 1H), 1.57-1.72 (m, J = 4.8 Hz, | | |
| | 1H), 1.28-1.56 (m, 3H), 1.08-1.26 (m, J = 11.7, 6.4 Hz, 8H), | | |
| | 0.98-1.07 (m, J = 11.0, 6.5 Hz, 5H), 0.90 (t, J = 6.8 Hz, 2.25H), | | |
| | 0.79-0.86 (m, 0.75H), 0.69-0.76 (m, 3H). | | |
| 31 | ¹H NMR (DMSO-d₆) (Rotamers present in NMR) d: 11.33 (s, 2H), | 7.33^{a} | 745.1 |
| | 8.97-9.12 (m, 1H), 8.61-8.84 (m, 1H), 8.09 (br. s, 1H), 7.94-8.03 (m, | | (M + H)⁺ |
| | 1H), 7.64-7.72 (m, 2H), 7.59 (s, 1H), 7.36 (t, J = 7.7 Hz, 1H), | | |
| | 7.22-7.30 (m, 2H), 7.11-7.18 (m, 2H), 4.47-4.72 (m, 2H), 4.10 (q, J = 6.9 Hz, | | |
| | 2H), 3.88-4.04 (m, 1H), 3.01 (s, 3H), 2.87 (s, 3H), 2.57-2.71 (m, | | |
| | 1H), 1.58-1.72 (m, 1H), 1.40-1.55 (m, 2H), 1.36 (t, J = 6.9 Hz, 3H), | | |
| | 1.05-1.25 (m, 7H), 0.85-0.98 (m, 3H), 0.71 (t, J = 6.8 Hz, 3H). | | |
| 32 | ¹H NMR (DMSO-d₆) (Rotamers present in NMR) d: 13.50 (br. s, | 6.95^{a} | ND |
| | 1H), 12.79 (br. s, 1H), 12.34-12.61 (m, 1H), 9.10 (d, J = 7.8 Hz, 1H), | | |
| | 8.68-9.02 (m, 2H), 8.07-8.15 (m, 1H), 7.98 (d, J = 8.3 Hz, 2H), | | |
| | 7.61-7.71 (m, 2H), 7.56 (s, 1H), 7.19-7.35 (m, 4H), 4.97 (s, 2H), | | |
| | 4.78-4.87 (m, 1H), 4.52-4.76 (m, 2H), 3.90-4.02 (m, 1H), 2.99 (s, 3H), | | |
| | 2.85 (s, 3H), 2.73-2.91 (m, 2H), 2.58-2.69 (m, 1H), 1.58-1.73 (m, | | |
| | 1H), 1.29-1.56 (m, 3H), 1.06-1.27 (m, J = 14.1, 7.8 Hz, 6H), 0.94 (t, | | |
| | J = 6.7 Hz, 3H), 0.72 (t, J = 6.8 Hz, 3H). | | |
| 33 | ¹H NMR (DMSO-d₆) (Rotamers present in NMR) d: 12.20-13.80 (m, | 2.15^{b} | 817.1 |
| | 3H), 9.12 (d, J = 8.3 Hz, 1H), 8.89-9.04 (m, 1H), 8.72-8.87 (m, 1H), | | (M + H)⁺ |
| | 8.62 (d, J = 8.5 Hz, 1H), 8.27-8.38 (m, 1H), 8.17-8.26 (m, 2H), | | |
| | 8.04 (dd, J = 7.8, 1.3 Hz, 1H), 7.95 (d, J = 8.0 Hz, 1H), 7.56-7.72 (m, 4H), | | |
| | 7.50-7.55 (m, 1H), 7.18-7.26 (m, 2H), 4.96 (s, 2H), 4.77-4.84 (m, | | |
| | J = 6.5, 6.5, 6.5 Hz, 1H), 4.55-4.77 (m, 2H), 3.99-4.16 (m, 1H), | | |
| | 2.73-2.90 (m, 2H), 2.68-2.77 (m, 1H), 1.68-1.80 (m, 1H), 1.40-1.63 (m, | | |
| | 3H), 1.08-1.27 (m, 6H), 0.93-1.07 (m, 3H), 0.72 (t, J = 6.7 Hz, 3H). | | |
| 34 | ¹H NMR (DMSO-d₆) (Rotamers present in NMR) d: 13.13-13.86 (m, | 1.47^{b} | 817.1 |
| | 1H), 12.14-13.04 (m, 2H), 9.12 (d, J = 7.8 Hz, 1H), 8.74-9.06 (m, 2H), | | (M + H)⁺ |
| | 8.65 (s, 1H), 8.10-8.22 (m, 2H), 7.92-8.04 (m, 4H), 7.60-7.74 (m, | | |
| | 2H), 7.52-7.60 (m, 1H), 7.50 (s, 1H), 7.04-7.20 (m, 2H), 4.94 (s, | | |
| | 2H), 4.78-4.86 (m, 1H), 4.57-4.80 (m, 2H), 3.97-4.12 (m, 1H), | | |
| | 2.70-2.92 (m, 3H), 1.65-1.81 (m, 1H), 1.35-1.64 (m, J = 7.8 Hz, 3H), | | |
| | 1.07-1.32 (m, 6H), 1.01 (t, J = 7.2 Hz, 3H), 0.67-0.79 (m, 3H). | | |
| 35 | ¹H NMR (DMSO-d₆) (Rotamers present in NMR) d: 9.16 (d, J = 7.8 Hz, | 0.76^{k} | 866.6 |
| | 1H), 8.85 (br. s, 2H), 8.06-8.20 (m, 1H), 7.87-8.06 (m, 3H), | | (M + H)⁺ |
| | 7.58-7.67 (m, 1H), 7.54 (s, 1H), 7.41 (d, J = 8.3 Hz, 2H), | | |
| | 7.12-7.29 (m, 2H), 4.94 (s, 2H), 4.67-4.85 (m, 2H), 4.54-4.67 (m, 1H), 4.00 (br. | | |
| | s, 1H), 3.56-3.64 (m, 4H), 3.54 (br. s, 2H), 2.63-2.89 (m, 3H), | | |
| | 2.37 (d, J = 4.3 Hz, 4H), 1.69 (br. s, 1H), 1.48 (d, J = 7.3 Hz, 3H), | | |
| | 1.06-1.28 (m, 6H), 0.85-1.04 (m, 3H), 0.74 (t, J = 6.9 Hz, 3H). | | |
| 36 | ¹H NMR (DMSO-d₆) (Rotamers present in NMR)d: 13.49 (br. s, 1H), | 2.67^{b} | 824.3 |
| | 12.79 (br. s, 1H), 12.46 (br. s, 1H), 9.11 (d, J = 7.8 Hz, 1H), | | (M + H)⁺ |
| | 8.87-8.99 (m, 1H), 8.32-8.43 (m, 1H), 8.00 (d, J = 8.3 Hz, 2H), 7.74-7.87 (m, | | |
| | 1H), 7.68 (d, J = 8.3 Hz, 1H), 7.55-7.63 (m, 1H), 7.23-7.40 (m, 6H), | | |
| | 7.19 (q, J = 7.3 Hz, 1H), 4.97 (s, 2H), 4.76-4.87 (m, 1H), | | |
| | 4.48-4.75 (m, 2H), 3.66-3.81 (m, 1H), 2.71-2.92 (m, 2H), 2.54-2.60 (m, 1H), | | |
| | 1.35-1.69 (m, 9H), 1.05-1.30 (m, 7H), 0.84 (t, J = 7.0 Hz, 3H), | | |
| | 0.68-0.78 (m, 3H). | | |
| 37 | ¹H NMR (DMSO-d₆) (Rotamers present in NMR) d: 13.49 (br. s, | 7.36^{j} | 904.4 |
| | 1H), 12.81 (br. s, 1H), 12.56 (br. s, 1H), 9.01-9.24 (m, 2H), | | (M + H)⁺ |
| | 8.36-8.59 (m, 1H), 8.21-8.32 (m, J = 8.3 Hz, 1H), 8.13 (br. s, 1H), 8.00 (d, | | |
| | J = 8.3 Hz, 1H), 7.68 (dd, J = 8.3, 1.0 Hz, 1H), 7.59 (s, 1H), | | |
| | 7.35-7.46 (m, 2H), 7.29 (dd, J = 15.9, 3.6 Hz, 2H), 7.24 (d, J = 8.0 Hz, 2H), | | |
| | 4.99 (s, 2H), 4.78-4.86 (m, 1H), 4.43-4.76 (m, 2H), 4.23-4.35 (m, 1H), | | |
| | 4.02-4.16 (m, 1H), 3.84-3.95 (m, 1H), 3.58 (br. s, 3H), 2.73-2.90 (m, | | |
| | 2H), 2.65-2.73 (m, 1H), 1.30-1.70 (m, 4H), 1.20-1.30 (m, 3H), | | |
| | 1.02-1.20 (m, 6H), 0.73-0.88 (m, 3H), 0.69 (t, J = 6.8 Hz, 3H). | | |
| 38 | ¹H NMR (DMSO-d₆) (Rotamers present in NMR) d: 12.27-13.85 (m, | 0.98^{k} | 799.4 |
| | 3H), 9.12 (d, J = 7.8 Hz, 1H), 8.92-9.03 (m, 1H), 8.58-8.74 (m, 1H), | | (M + H)⁺ |
| | 8.15 (s, 1H), 7.99 (d, J = 8.3 Hz, 1H), 7.68 (dd, J = 8.3, 1.3 Hz, 1H), | | |
| | 7.59 (d, J = 1.0 Hz, 1H), 7.46-7.56 (m, 1H), 7.23-7.32 (m, 2H), | | |
| | 7.19 (dd, J = 8.4, 4.6 Hz, 2H), 4.98 (s, 2H), 4.81 (q, J = 7.1 Hz, 1H), | | |
| | 4.41-4.74 (m, 2H), 3.98-4.14 (m, 1H), 2.72-2.92 (m, 2H), 2.56-2.70 (m, | | |
| | 1H), 2.39 (s, 3H), 1.64-1.81 (m, 1H), 1.33-1.54 (m, 3H), | | |
| | 1.04-1.25 (m, 6H), 0.82-1.02 (m, 3H), 0.69 (t, J = 6.9 Hz, 3H). | | |
| 39 | ¹H NMR (DMSO-d₆) (Rotamers present in NMR) d: 13.04 (br. s, | 7.56^{j} | 811.4 |
| | 3H), 9.13 (d, J = 7.8 Hz, 1H), 8.55-8.98 (m, 2H), 8.07-8.14 (m, 1H), | | (M + H)⁺ |
| | 7.98 (d, J = 8.3 Hz, 2H), 7.64 (d, J = 8.3 Hz, 1H), 7.56 (s, 1H), | | |
| | 7.21-7.29 (m, 2H), 6.73-6.92 (m, 2H), 4.96 (s, 2H), 4.80 (q, J = 6.8 Hz, | | |
| | 1H), 4.51-4.76 (m, 2H), 3.92-4.05 (m, 1H), 3.80 (s, 3H), | | |
| | 2.72-2.91 (m, 2H), 2.60-2.72 (m, 1H), 2.41 (s, 3H), 1.60-1.77 (m, 1H), | | |
| | 1.31-1.57 (m, 3H), 1.06-1.29 (m, 6H), 0.96 (t, J = 6.8 Hz, 3H), 0.73 (t, | | |
| | J = 6.8 Hz, 3H). | | |
| 40 | ¹H NMR (DMSO-d₆) (Rotamers present in NMR) d: 13.49 (br. s, | 6.35^{j} | 782.3 |
| | 1H), 12.80 (br. s, 1H), 12.41 (br. s, 1H), 9.10 (d, J = 7.8 Hz, 1H), | | (M + H)⁺ |
| | 8.60-8.65 (m, 1H), 8.57 (d, J = 5.0 Hz, 1H), 8.18 (br. s, 1H), | | |
| | 7.95-8.02 (m, 1H), 7.79 (d, J = 4.5 Hz, 1H), 7.65 (d, J = 8.0 Hz, 1H), 7.56 (s, | | |
| | 1H), 7.24-7.32 (m, 1H), 7.22 (s, 1H), 7.10 (s, 1H), 6.97 (s, 1H), | | |
| | 4.98 (s, 2H), 4.77-4.86 (m, 1H), 4.50-4.74 (m, 2H), 3.98-4.13 (m, 1H), | | |
| | 2.73-2.90 (m, 2H), 2.60-2.72 (m, 1H), 2.40 (br. s, 3H), 1.65-1.78 (m, | | |
| | 1H), 1.34-1.58 (m, 3H), 1.07-1.27 (m, 6H), 0.85-1.01 (m, 3H), | | |
| | 0.67-0.76 (m, 3H). | | |
| 41 | ¹H NMR (DMSO-d₆) (Rotamers present in NMR) d: 12.66 (br. s, | 0.74^{k} | 880.5 |
| | 2H), 9.13 (d, J = 7.8 Hz, 1H), 8.90 (br. s, 1H), 8.73 (br. s, 1H), | | (M + H)⁺ |
| | 8.14 (br. s, 1H), 7.86-8.02 (m, 2H), 7.64 (d, J = 8.0 Hz, 1H), 7.57 (s, 1H), | | |
| | 7.25 (dd, J = 12.3, 3.5 Hz, 4H), 4.96 (s, 2H), 4.77-4.87 (m, 1H), | | |
| | 4.69 (dd, J = 12.3, 6.0 Hz, 1H), 4.51-4.63 (m, 1H), 4.01 (br. s, 1H), | | |
| | 3.61 (br. s, 6H), 2.71-2.92 (m, 2H), 2.67 (br. s, 1H), 2.51 (br. s, 4H), | | |
| | 2.43 (br. s, 3H), 1.70 (br. s, 1H), 1.32-1.59 (m, 3H), 1.06-1.23 (m, 6H), | | |
| | 0.96 (br. s, 3H), 0.73 (t, J = 6.8 Hz, 3H). | | |
| 42 | ¹H NMR (DMSO-d₆) (Rotamers present in NMR) d: 12.94 (br. s, | 7.16^{j} | 827.3 |
| | 3H), 9.13 (d, J = 7.8 Hz, 1H), 8.67-8.95 (m, 2H), 8.12 (s, 1H), 7.97 (d, | | (M + H)⁺ |
| | J = 8.3 Hz, 1H), 7.66-7.76 (m, 1H), 7.57-7.66 (m, 1H), 7.52 (s, 1H), | | |
| | 7.27-7.35 (m, 1H), 7.19-7.26 (m, 2H), 6.87-6.99 (m, 1H), 4.95 (s, | | |
| | 2H), 4.76-4.86 (m, 1H), 4.55-4.77 (m, 2H), 3.98 (t, J = 8.9 Hz, 1H), | | |
| | 3.82 (s, 3H), 3.74 (s, 3H), 2.73-2.90 (m, 2H), 2.65-2.73 (m, 1H), | | |
| | 1.62-1.78 (m, 1H), 1.31-1.61 (m, 3H), 1.04-1.30 (m, 6H), 0.97 (t, | | |
| | J = 7.2 Hz, 3H), 0.74 (t, J = 6.8 Hz, 3H). | | |
| 43 | ¹H NMR (DMSO-d₆) (Rotamers present in NMR) d: 12.20-13.75 (m, | 1.10^{k} | 789.3 |
| | 3H), 9.12 (d, J = 7.8 Hz, 1H), 8.79-8.94 (m, 1H), 8.63-8.77 (m, 1H), | | (M + H)⁺ |
| | 7.95-8.06 (m, 2H), 7.68 (dd, J = 8.2, 1.4 Hz, 1H), 7.60 (s, 1H), | | |
| | 7.29 (dd, J = 17.1, 3.5 Hz, 2H), 4.97 (s, 2H), 4.76-4.86 (m, 1H), | | |
| | 4.48-4.73 (m, 2H), 4.06-4.16 (m, 0.2H), 3.78-3.92 (m, 0.8H), 2.73-2.91 (m, | | |
| | 2H), 2.53-2.59 (m, 1H), 1.61-1.73 (m, 1H), 1.49-1.60 (m, 2H), | | |
| | 1.35-1.48 (m, 4H), 1.22-1.35 (m, 5H), 1.04-1.21 (m, 7H), 0.90 (t, J = 7.2 Hz, | | |
| | 3H), 0.85 (t, J = 7.3 Hz, 3H), 0.79 (t, J = 7.2 Hz, 3H), 0.72 (t, J = 6.9 Hz, | | |
| | 3H). | | |
| 44 | ¹H NMR (DMSO-d₆) (Rotamers present in NMR) d: 9.65-9.79 (m, | 7.26^{j} | 827.3 |
| | 1H), 8.90-9.01 (m, 1H), 8.08 (br. s, 1H), 7.82-7.94 (m, 2H), 7.57 (d, | | (M + H)⁺ |
| | J = 9.0 Hz, 1H), 7.50 (s, 1H), 7.20-7.25 (m, 4H), 6.60-6.64 (m, 1H), | | |
| | 6.49-6.58 (m, 1H), 4.61-4.76 (m, 3H), 4.44-4.60 (m, 2H), | | |
| | 3.89-4.03 (m, 1H), 3.82 (s, 3H), 3.80 (s, 3H), 2.62-2.76 (m, 2H), 2.58-2.61 (m, | | |
| | 1H), 1.59-1.73 (m, 1H), 1.28-1.58 (m, 3H), 1.05-1.27 (m, 6H), | | |
| | 0.95 (t, J = 6.5 Hz, 3H), 0.71 (t, J = 6.0 Hz, 3H). | | |
| 45 | ¹H NMR (DMSO-d₆) (Rotamers present in NMR) d: 11.26 (br. s, | 1.05^{k} | 652.3 |
| | 2H), 8.96 (br. s, 1H), 8.69 (br. s, 1H), 7.95-8.06 (m, 1H), | | (M + H)⁺ |
| | 7.57-7.71 (m, 2H), 7.21-7.29 (m, 1H), 7.08-7.19 (m, 2H), 4.44-4.69 (m, 2H), | | |
| | 4.11 (q, J = 7.0 Hz, 2H), 3.80-3.90 (m, 1H), 2.54-2.62 (m, 1H), | | |
| | 2.29-2.43 (m, 1H), 1.60-1.71 (m, 1H), 1.47-1.60 (m, 4H), 1.41-1.47 (m, | | |
| | 2H), 1.37 (t, J = 6.9 Hz, 3H), 1.05-1.25 (m, 7H), 0.79-0.94 (m, 9H), | | |
| | 0.71 (t, J = 7.0 Hz, 3H). | | |
| 46 | ¹H NMR (DMSO-d₆) (Rotamers present in NMR) d: 11.27 (br. s, | 1.04^{k} | 690.3 |
| | 2H), 8.97-9.11 (m, 1H), 8.56-8.74 (m, 1H), 8.14 (s, 1H), 7.67 (d, | | (M + H)⁺ |
| | J = 13.3 Hz, 1H), 7.61 (s, 1H), 7.46-7.56 (m, 1H), 7.10-7.25 (m, 5H), | | |
| | 4.38-4.71 (m, 2H), 4.11 (q, J = 7.0 Hz, 2H), 3.98-4.06 (m, 1H), | | |
| | 2.54-2.71 (m, 1H), 2.41 (s, 3H), 1.64-1.79 (m, 1H), 1.41-1.52 (m, 3H), | | |
| | 1.37 (t, J = 6.9 Hz, 3H), 1.03-1.21 (m, 6H), 0.89-1.01 (m, 3H), 0.69 (t, | | |
| | J = 6.9 Hz, 3H). | | |
| 47 | ¹H NMR (DMSO-d₆) (Rotamers present in NMR) d: 11.25 (br. s, | 0.99^{k} | 688.3 |
| | 2H), 9.02 (t, J = 5.9 Hz, 1H), 8.70-8.94 (m, 1H), 8.10 (s, 1H), | | (M + H)⁺ |
| | 7.94-8.03 (m, 2H), 7.65 (d, J = 13.3 Hz, 1H), 7.56-7.60 (m, 1H), | | |
| | 7.20-7.25 (m, 1H), 7.07-7.18 (m, 2H), 7.02 (d, J = 8.8 Hz, 2H), 4.48-4.71 (m, | | |
| | 2H), 4.11 (q, J = 7.0 Hz, 2H), 3.90-4.02 (m, 1H), 3.83 (s, 3H), | | |
| | 2.54-2.64 (m, 1H), 1.60-1.77 (m, 1H), 1.40-1.53 (m, 2H), 1.37 (t, J = 6.9 Hz, | | |
| | 3H), 1.03-1.26 (m, 7H), 0.96 (t, J = 7.0 Hz, 3H), 0.71 (t, J = 6.9 Hz, | | |
| | 3H). | | |
| 48 | ¹H NMR (DMSO-d₆) (Rotamers present in NMR) d: 12.95 (br. s, | 1.08^{k} | 731.4 |
| | 1H), 12.55 (br. s, 1H), 8.89 (d, J = 7.8 Hz, 1H), 8.82 (br. s, 1H), | | (M + H)⁺ |
| | 8.63-8.75 (m, 1H), 7.95-8.07 (m, 2H), 7.59-7.67 (m, 2H), 7.28 (dd, | | |
| | J = 16.3, 3.5 Hz, 2H), 4.74-4.85 (m, 1H), 4.50-4.73 (m, 2H), 4.30 (q, | | |
| | J = 7.0 Hz, 2H), 3.86 (t, J = 9.5 Hz, 1H), 2.74-2.95 (m, 2H), | | |
| | 2.54-2.62 (m, 1H), 2.31-2.40 (m, 1H), 1.60-1.72 (m, 1H), 1.38-1.59 (m, 9H), | | |
| | 1.04-1.27 (m, 7H), 0.91 (t, J = 6.9 Hz, 3H), 0.78-0.88 (m, 6H), 0.73 (t, | | |
| | J = 6.9 Hz, 3H). | | |
| 49 | ¹H NMR (DMSO-d₆) (Rotamers present in NMR) d: 12.93 (br. s, | 1.07^{k} | 769.4 |
| | 1H), 12.51 (br. s, 1H), 8.93 (m, 1H), 8.88 (d, J = 7.8 Hz, 1H), 8.67 (br. | | (M + H)⁺ |
| | s, 1H), 8.16 (s, 1H), 8.00 (d, J = 8.0 Hz, 1H), 7.57-7.66 (m, 2H), | | |
| | 7.46-7.56 (m, 1H), 7.22-7.33 (m, 2H), 7.15-7.22 (m, 2H), 4.76-4.86 (m, | | |
| | 1H), 4.43-4.74 (m, 2H), 4.31 (q, J = 6.9 Hz, 2H), 3.96-4.11 (m, 1H), | | |
| | 2.73-2.97 (m, 2H), 2.55-2.70 (m, 1H), 2.39 (s, 3H), 1.66-1.79 (m, | | |
| | 1H), 1.35-1.52 (m, 6H), 1.03-1.24 (m, 6H), 0.87-1.01 (m, 3H), | | |
| | 0.70 (t, J = 7.0 Hz, 3H). | | |
| 50 | ¹H NMR (DMSO-d₆) (Rotamers present in NMR) d: 9.01 (t, J = 5.6 Hz, | 8.25^{j} | 727.4 |
| | 1H), 8.76-8.92 (m, 1H), 8.05 (s, 1H), 7.82 (d, J = 8.5 Hz, 2H), | | (M + H)⁺ |
| | 7.67 (d, J = 13.1 Hz, 1H), 7.45 (br. s, 1H), 7.19 (d, J = 3.5 Hz, 1H), | | |
| | 7.15 (dd, J = 14.4, 1.6 Hz, 1H), 6.97 (d, J = 3.0 Hz, 1H), 6.44-6.55 (m, | | |
| | 2H), 4.47-4.78 (m, 2H), 3.99 (q, J = 6.6 Hz, 2H), 3.84-3.95 (m, 1H), | | |
| | 3.13-3.31 (m, 4H), 2.61-2.75 (m, 1H), 1.82-1.98 (m, 4H), | | |
| | 1.57-1.71 (m, 1H), 1.35-1.54 (m, 2H), 1.29 (t, J = 6.9 Hz, 3H), 1.04-1.25 (m, | | |
| | 7H), 0.95 (t, J = 7.2 Hz, 3H), 0.71 (t, J = 6.9 Hz, 3H). | | |
| 51 | ¹H NMR (DMSO-d₆) (Rotamers present in NMR) d: 12.95 (br. s, | 1.06^{k} | 767.4 |
| | 1H), 12.55 (br. s, 1H), 8.69-9.04 (m, J = 6.5 Hz, 3H), 8.12 (br. s, 1H), | | (M + H)⁺ |
| | 7.97 (t, J = 8.5 Hz, 3H), 7.47-7.64 (m, 2H), 7.23 (br. s, 2H), | | |
| | 6.90-7.05 (m, 2H), 4.77-4.90 (m, 1H), 4.51-4.77 (m, 2H), 4.28 (q, J = 6.8 Hz, | | |
| | 2H), 3.90-4.04 (m, 1H), 3.82 (s, 3H), 2.75-3.00 (m, 2H), | | |
| | 2.63-2.74 (m, 1H), 1.59-1.78 (m, 1H), 1.47 (t, J = 6.7 Hz, 3H), 1.38-1.57 (m, | | |
| | 2H), 1.05-1.30 (m, 7H), 0.86-1.02 (m, 3H), 0.67-0.78 (m, 3H). | | |
| 52 | ¹H NMR (DMSO-d₆) (Rotamers present in NMR) d: 13.49 (br. s, | 1.13^{k} | 839.4 |
| | 1H), 12.80 (br. s, 1H), 12.45 (br. s, 1H), 9.10 (d, J = 7.8 Hz, 1H), | | (M + H)⁺ |
| | 8.88-9.04 (m, 1H), 8.06-8.16 (m, 1H), 7.98 (d, J = 8.3 Hz, 1H), | | |
| | 7.66 (dd, J = 8.3, 1.3 Hz, 1H), 7.57 (s, 1H), 7.24-7.30 (m, 2H), 7.23 (s, | | |
| | 1H), 7.10 (s, 1H), 6.92-7.00 (m, 2H), 4.97 (s, 2H), 4.76-4.87 (m, | | |
| | 1H), 4.49-4.76 (m, 2H), 3.91-4.08 (m, 1H), 3.81 (s, 3H), | | |
| | 3.59-3.74 (m, 1H), 2.73-2.92 (m, 2H), 2.59-2.71 (m, 1H), 1.63-1.75 (m, 1H), | | |
| | 1.30-1.57 (m, 3H), 1.04-1.25 (m, 12H), 0.86-1.01 (m, 3H), 0.72 (t, | | |
| | J = 6.9 Hz, 3H). | | |
| 53 | ¹H NMR (DMSO-d₆) (Rotamers present in NMR) d: 12.26-13.84 (m, | 1.14^{k} | 827.4 |
| | 3H), 9.13 (d, J = 7.8 Hz, 1H), 8.91-9.05 (m, 1H), 8.07-8.22 (m, 1H), | | (M + H)⁺ |
| | 7.98 (d, J = 8.3 Hz, 1H), 7.85-7.94 (m, 1H), 7.66 (dd, J = 8.2, 1.1 Hz, | | |
| | 1H), 7.57 (d, J = 1.0 Hz, 1H), 7.34 (dd, J = 10.8, 2.5 Hz, 1H), | | |
| | 7.25-7.31 (m, 2H), 6.95-7.24 (m, 2H), 4.97 (s, 2H), 4.80 (q, J = 7.0 Hz, 1H), | | |
| | 4.48-4.74 (m, 2H), 3.95-4.12 (m, 1H), 3.54-3.67 (m, 1H), | | |
| | 2.72-2.90 (m, 2H), 2.58-2.72 (m, 1H), 1.63-1.79 (m, 1H), 1.36-1.58 (m, 3H), | | |
| | 1.05-1.26 (m, 12H), 0.84-1.03 (m, 3H), 0.71 (t, J = 6.9 Hz, 3H). | | |
| 54 | ¹H NMR (DMSO-d₆) (Rotamers present in NMR) d: 8.95 (d, J = 6.0 Hz, | 2.15^{b} | 781.3 |
| | 1H), 8.79-8.90 (m, 1H), 8.12 (br. s, 1H), 7.99 (d, J = 8.0 Hz, 2H), | | (M + H)⁺ |
| | 7.53-7.62 (m, 2H), 7.20-7.29 (m, 2H), 6.84-6.91 (m, 1H), | | |
| | 6.74-6.84 (m, 1H), 4.45-4.75 (m, 3H), 4.18-4.39 (m, J = 8.3 Hz, 3H), | | |
| | 3.91-4.05 (m, 1H), 3.79 (s, 3H), 2.56-2.88 (m, 3H), 2.42 (s, 3H), 1.61-1.76 (m, | | |
| | 1H), 1.49 (t, J = 6.9 Hz, 3H), 1.32-1.45 (m, 2H), 1.06-1.30 (m, 7H), | | |
| | 0.96 (t, J = 5.8 Hz, 3H), 0.73 (t, J = 6.9 Hz, 3H). | | |
| 55 | ¹H NMR (DMSO-d₆) (Rotamers present in NMR) d: 11.32 (br. s, | 6.74^{a} | 554.2 |
| | 2H), 9.21 (t, J = 5.5 Hz, 0.3H), 9.04 (t, J = 5.9 Hz, 0.7H), 8.37-8.44 (m, | | (M − tBuNCO + 1) |
| | 0.7H), 8.34 (t, J = 5.9 Hz, 0.3H), 8.05 (s, 0.3H), 7.98 (s, 0.7H), | | |
| | 7.67 (d, J = 13.3 Hz, 1H), 7.61 (s, 0.7H), 7.44 (s, 0.3H), 7.25 (d, J = 3.8 Hz, | | |
| | 1H), 7.18-7.24 (m, 1H), 7.12-7.18 (m, 2H), 4.44-4.69 (m, 2H), | | |
| | 4.15-4.25 (m, 0.3H), 4.06-4.17 (m, 2H), 3.74 (t, J = 8.8 Hz, 0.7H), | | |
| | 2.53-2.58 (m, 1H), 1.55-1.69 (m, J = 3.5 Hz, 1H), 1.45-1.54 (m, 1H), | | |
| | 1.37 (t, J = 6.9 Hz, 3H), 1.29-1.44 (m, 2H), 1.25 (s, 3H), 1.22 (s, 6H), | | |
| | 1.05-1.19 (m, 6H), 0.90 (t, J = 7.0 Hz, 2H), 0.83 (t, J = 7.2 Hz, 1H), | | |
| | 0.66-0.74 (m, 3H). | | |
| 56 | ¹H NMR (MEOH-d₄) (Rotamers present in NMR) d: 8.24-8.46 (m, | 7.12^{a} | 715.3 |
| | 1H), 7.95-8.07 (m, 1H), 7.79 (d, J = 13.6 Hz, 1H), 7.59-7.68 (m, 1H), | | (M + H)⁺ |
| | 7.38 (d, J = 7.5 Hz, 2H), 7.27-7.34 (m, 1H), 7.22-7.28 (m, 5H), | | |
| | 7.12-7.21 (m, 1H), 7.01 (dd, J = 10.2, 3.6 Hz, 1H), 4.65-4.80 (m, 2H), | | |
| | 4.30-4.37 (m, 0.3H), 4.16 (q, J = 7.2 Hz, 2H), 3.75-3.88 (m, 0.7H), | | |
| | 2.44-2.64 (m, 1H), 1.69-1.86 (m, J = 7.8 Hz, 1H), 1.48-1.68 (m, 8H), | | |
| | 1.45 (t, J = 7.0 Hz, 3H), 1.12-1.35 (m, 7H), 0.96 (t, J = 7.0 Hz, 2H), | | |
| | 0.86-0.92 (m, 1H), 0.78-0.86 (m, 3H). | | |
| 57 | ¹H NMR (DMSO-d₆) (Rotamers present in NMR) d: 9.13-9.22 (m, | 1.03 | 743.6 |
| | 0.1H), 9.02 (t, J = 5.6 Hz, 0.9H), 8.65-8.90 (m, 0.9H), 8.56-8.64 (m, | JDalton | (M + H)⁺ |
| | 0.1H), 8.25 (s, 0.1H), 8.08 (br. s, 0.9H), 7.86 (d, J = 7.5 Hz, 2H), | | |
| | 7.66 (d, J = 13.3 Hz, 1H), 7.58 (br. s, 1H), 7.24 (d, J = 3.5 Hz, 1H), 7.17 (br. | | |
| | s, 1H), 7.08-7.15 (m, 1H), 6.92 (d, J = 8.5 Hz, 2H), 4.49-4.74 (m, 2H), | | |
| | 4.05-4.15 (m, 2H), 3.89-4.00 (m, 1H), 3.61-3.75 (m, 4H), | | |
| | 3.19-3.26 (m, 4H), 2.62-2.75 (m, 1H), 1.58-1.74 (m, 1H), 1.41-1.54 (m, 2H), | | |
| | 1.36 (t, J = 6.9 Hz, 3H), 1.02-1.25 (m, 7H), 0.83-1.01 (m, 3H), 0.71 (t, | | |
| | J = 6.7 Hz, 3H). | | |
| 58 | ¹H NMR (DMSO-d₆) (Rotamers present in NMR) d: 9.08 (t, J = 5.8 Hz, | 8.07^{j} | 723.3 |
| | 1H), 8.81-9.01 (m, 1H), 8.03-8.18 (m, J = 9.8 Hz, 2H), 7.74 (d, | | (M + H)⁺ |
| | J = 8.3 Hz, 2H), 7.67 (d, J = 12.3 Hz, 1H), 7.52 (t, J = 2.1 Hz, 2H), | | |
| | 7.42-7.48 (m, 1H), 7.19 (d, J = 3.5 Hz, 1H), 7.15 (dd, J = 13.4, 1.6 Hz, 2H), | | |
| | 6.98 (d, J = 3.3 Hz, 1H), 6.28-6.36 (m, 2H), 4.47-4.77 (m, 2H), | | |
| | 3.95-4.05 (m, 3H), 2.65-2.80 (m, 1H), 1.58-1.78 (m, 1H), 1.37-1.56 (m, | | |
| | 2H), 1.30 (t, J = 6.9 Hz, 3H), 1.04-1.24 (m, 7H), 0.96 (t, J = 7.2 Hz, | | |
| | 3H), 0.70 (t, J = 6.9 Hz, 3H). | | |
| 59 | ¹H NMR (DMSO-d₆) (Rotamers present in NMR) d: 8.94-9.04 (m, | 7.34^{j} | 741.3 |
| | 1H), 8.11 (br. s, 1H), 8.05 (d, J = 8.3 Hz, 2H), 7.80 (d, J = 8.8 Hz, 2H), | | (M + H)⁺ |
| | 7.67 (d, J = 12.8 Hz, 1H), 7.43 (s, 1H), 7.18 (d, J = 3.5 Hz, 1H), | | |
| | 7.15 (dd, J = 13.9, 2.4 Hz, 2H), 6.97 (d, J = 3.3 Hz, 1H), 4.47-4.76 (m, 2H), | | |
| | 3.92-4.05 (m, 3H), 3.80 (t, J = 7.0 Hz, 2H), 2.61-2.75 (m, 1H), | | |
| | 2.50-2.53 (m, 2H), 1.91-2.12 (m, 2H), 1.60-1.76 (m, 1H), 1.37-1.58 (m, | | |
| | 2H), 1.30 (t, J = 6.9 Hz, 3H), 1.05-1.26 (m, 7H), 0.96 (t, J = 7.2 Hz, | | |
| | 3H), 0.71 (t, J = 7.0 Hz, 3H). | | |
| 60 | ¹H NMR (DMSO-d₆) (Rotamers present in NMR) d: 9.02 (t, J = 5.9 Hz, | 7.60^{j} | 729.3 |
| | 1H), 8.05 (s, 1H), 7.67 (d, J = 12.5 Hz, 1H), 7.48-7.57 (m, 1H), | | (M + H)⁺ |
| | 7.43 (br. s, 1H), 7.25-7.35 (m, 1H), 7.19 (d, J = 3.8 Hz, 1H), 7.15 (dd, | | |
| | J = 13.4, 1.6 Hz, 2H), 6.97 (d, J Hz, 1H), 6.67 (d, J = 8.8 Hz, 1H), | | |
| | 4.46-4.80 (m, 2H), 4.18 (t, J = 4.4 Hz, 2H), 3.87-4.05 (m, 3H), 3.38 (t, | | |
| | J = 4.3 Hz, 2H), 2.92 (s, 3H), 2.62-2.77 (m, 1H), 1.56-1.71 (m, 1H), | | |
| | 1.35-1.52 (m, 2H), 1.30 (t, J = 6.9 Hz, 3H), 1.03-1.23 (m, 7H), 0.94 (t, | | |
| | J = 7.2 Hz, 3H), 0.69 (t, J = 7.0 Hz, 3H). | | |
| 61 | ¹H NMR (DMSO-d₆) (Rotamers present in NMR) d: 8.76-9.11 (m, | 6.24^{a} | 798.3 |
| | 1H), 8.13-8.25 (m, 1H), 8.02 (d, J = 13.6 Hz, 1H), 7.33-7.44 (m, 2H), | | (M + H)⁺ |
| | 7.11-7.18 (m, 4H), 7.09 (d, J = 1.5 Hz, 1H), 6.97 (d, J = 7.8 Hz, 1H), | | |
| | 5.66 (dd, J = 16.2, 6.1 Hz, 2H), 5.52-5.60 (m, 1H), 4.09 (q, J = 7.0 Hz, | | |
| | 3H), 3.74-3.89 (m, 1H), 2.56-2.73 (m, 1H), 2.34 (s, 3H), | | |
| | 1.62-1.78 (m, 1H), 1.38-1.54 (m, 4H), 1.35 (t, J = 7.0 Hz, 3H), 1.13-1.24 (m, | | |
| | 2H), 0.99-1.12 (m, 3H), 0.69-0.96 (m, 3H), 0.62 (t, J = 6.9 Hz, 3H). | | |
| 62 | ¹H NMR (DMSO-d₆) (Rotamers present in NMR) d: 8.76-9.02 (m, | 8.36^{j} | 745.3 |
| | 1H), 8.06 (s, 1H), 7.59-7.71 (m, 3H), 7.48 (br. s, 1H), 7.19 (d, J = 3.5 Hz, | | (M + H)⁺ |
| | 1H), 7.14 (dd, J = 13.7, 1.6 Hz, 2H), 7.00 (d, J = 3.0 Hz, 1H), | | |
| | 6.63 (t, J = 8.8 Hz, 1H), 4.44-4.74 (m, 2H), 3.98-4.05 (m, 2H), | | |
| | 3.88-3.97 (m, 1H), 3.40-3.44 (m, 4H), 2.64-2.76 (m, 1H), 1.78-1.95 (m, 4H), | | |
| | 1.57-1.74 (m, 1H), 1.36-1.53 (m, 2H), 1.31 (t, J = 6.9 Hz, 3H), | | |
| | 1.04-1.28 (m, 7H), 0.95 (t, J = 7.2 Hz, 3H), 0.71 (t, J = 6.9 Hz, 3H). | | |
| 63 | ¹H NMR (DMSO-d₆) (Rotamers present in NMR) d: 9.03 (t, J = 6.1 Hz, | 8.14^{j} | 727.3 |
| | 1H), 8.12 (s, 1H), 7.64 (d, J = 13.1 Hz, 1H), 7.50 (br. s, 1H), | | (M + H)⁺ |
| | 7.26-7.36 (m, 2H), 7.19 (d, J = 3.5 Hz, 1H), 7.14 (dd, J = 13.7, 2.1 Hz, 2H), | | |
| | 7.02-7.05 (m, J = 0.5 Hz, 1H), 7.01 (d, J = 3.5 Hz, 1H), 6.83 (dd, | | |
| | J = 9.0, 2.8 Hz, 1H), 4.46-4.78 (m, 2H), 3.89-4.09 (m, 3H), | | |
| | 3.13-3.29 (m, 4H), 2.62-2.74 (m, 1H), 1.95 (t, J = 6.5 Hz, 4H), 1.61-1.77 (m, | | |
| | 1H), 1.36-1.54 (m, 2H), 1.31 (t, J = 7.0 Hz, 3H), 1.04-1.25 (m, 7H), | | |
| | 0.96 (t, J = 7.0 Hz, 3H), 0.70 (t, J = 7.0 Hz, 3H). | | |
| 64 | ¹H NMR (DMSO-d₆) (Rotamers present in NMR) d: 8.96-9.08 (m, | 0.79^{k} | 771.3 |
| | 1H), 8.52-8.84 (m, 1H), 8.12 (br. s, 1H), 7.87-7.97 (m, 1H), 7.66 (d, | | (M + H)⁺ |
| | J = 12.8 Hz, 1H), 7.49 (br. s, 1H), 7.24-7.32 (m, 2H), 7.20 (d, J = 3.5 Hz, | | |
| | 1H), 7.15 (dd, J = 14.2, 1.9 Hz, 1H), 6.99-7.07 (m, 1H), | | |
| | 4.45-4.71 (m, 2H), 4.03 (q, J = 6.4 Hz, 3H), 3.59 (t, J = 4.5 Hz, 4H), | | |
| | 3.48-3.53 (m, 2H), 2.63-2.70 (m, 1H), 2.46 (s, 3H), 2.34-2.42 (m, 4H), | | |
| | 1.62-1.77 (m, 1H), 1.37-1.57 (m, 3H), 1.32 (t, J = 6.9 Hz, 3H), | | |
| | 1.04-1.25 (m, 6H), 0.86-1.01 (m, 3H), 0.71 (t, J = 6.8 Hz, 3H). | | |
| 65 | ¹H NMR (DMSO-d₆) (Rotamers present in NMR) d: 9.16 (t, J = 5.6 Hz, | 0.90^{k} | 639.2 |
| | 0.7H), 9.03 (t, J = 5.5 Hz, 0.3H), 8.41 (t, J = 5.6 Hz, 0.7H), 8.33 (t, | | (M + H)⁺ |
| | J = 5.5 Hz, 0.3H), 8.12 (s, 0.3H), 7.99 (s, 0.7H), 7.67 (d, J = 13.3 Hz, | | |
| | 1H), 7.59-7.63 (m, 1H), 7.37-7.49 (m, 1H), 7.26 (d, J = 3.5 Hz, 1H), | | |
| | 7.15-7.18 (m, 1H), 7.12-7.17 (m, 1H), 4.45-4.70 (m, 2H), | | |
| | 4.18-4.27 (m, 0.3H), 4.12 (q, J = 7.0 Hz, 2H), 3.74-3.85 (m, 0.7H), | | |
| | 3.48-3.63 (m, 1H), 2.54-2.59 (m, 1H), 1.55-1.70 (m, 1H), 1.41-1.55 (m, 2H), | | |
| | 1.37 (t, J = 6.9 Hz, 3H), 1.09-1.23 (m, 7H), 1.07 (d, J = 6.5 Hz, 6H), | | |
| | 0.89 (t, J = 7.3 Hz, 2.1H), 0.83 (t, J = 7.3 Hz, 0.9H), 0.70 (t, J = 6.3 Hz, | | |
| | 3H). | | |
| 66 | ¹H NMR (DMSO-d₆) (Rotamers present in NMR) d: 12.96 (br. s, | 1.13^{k} | 809.3 |
| | 1H), 12.54 (br. s, 1H), 8.90-8.98 (m, 1H), 8.87 (d, J = 7.8 Hz, 1H), | | (M + H)⁺ |
| | 8.67-8.68 (m, 1H), 8.64-8.80 (m, 1H), 8.12 (br. s, 1H), 7.98 (d, J = 8.0 Hz, | | |
| | 1H), 7.88 (br. s, 1H), 7.61 (d, J = 8.0 Hz, 1H), 7.58 (s, 1H), | | |
| | 7.28-7.30 (m, 1H), 7.24-7.27 (m, 1H), 6.95 (d, J = 2.5 Hz, 1H), | | |
| | 6.75-6.85 (m, 1H), 4.77-4.85 (m, 1H), 4.52-4.74 (m, 2H), 4.29 (q, J = 6.8 Hz, | | |
| | 2H), 3.92-4.06 (m, 1H), 3.81 (s, 3H), 3.39-3.76 (m, 1H), | | |
| | 2.74-2.96 (m, 1H), 2.60-2.66 (m, 1H), 1.63-1.78 (m, 1H), 1.47 (t, J = 7.0 Hz, | | |
| | 3H), 1.38-1.51 (m, 2H), 1.04-1.26 (m, 13H), 0.87-1.03 (m, 3H), | | |
| | 0.72 (t, J = 6.9 Hz, 3H). | | |
| 67 | ¹H NMR (DMSO-d₆) (Rotamers present in NMR) d: 12.95 (br. s, | 1.15^{k} | 797.3 |
| | 1H), 12.54 (br. s, 1H), 8.91-9.00 (m, 1H), 8.87 (d, J = 7.8 Hz, 1H), | | (M + H)⁺ |
| | 8.58-8.81 (m, 1H), 8.15 (br. s, 1H), 7.99 (d, J = 8.0 Hz, 1H), | | |
| | 7.88-7.94 (m, 1H), 7.61 (d, J = 8.3 Hz, 1H), 7.59 (s, 1H), 7.34 (dd, J = 10.9, | | |
| | 2.6 Hz, 1H), 7.29 (d, J = 3.8 Hz, 1H), 7.26 (d, J = 3.8 Hz, 1H), | | |
| | 7.07-7.17 (m, 1H), 4.77-4.86 (m, 1H), 4.49-4.75 (m, 2H), 4.30 (q, J = 7.0 Hz, | | |
| | 2H), 3.95-4.09 (m, 1H), 3.52-3.66 (m, 1H), 2.74-2.96 (m, 2H), | | |
| | 2.56-2.66 (m, 1H), 1.64-1.78 (m, 1H), 1.47 (t, J = 7.0 Hz, 3H), | | |
| | 1.39-1.56 (m, 2H), 1.08-1.26 (m, 13H), 0.88-1.01 (m, 3H), 0.72 (t, J = 6.9 Hz, | | |
| | 3H). | | |
| 68 | ¹H NMR (DMSO-d₆) (Rotamers present in NMR) d: 9.01-9.15 (m, | 1.13^{k} | 718.3 |
| | 1H), 8.54-8.81 (m, 1H), 8.07-8.19 (m, 1H), 7.86-7.97 (m, 1H), | | (M + H)⁺ |
| | 7.66 (d, J = 13.3 Hz, 1H), 7.57-7.63 (m, 1H), 7.36 (dd, J = 11.0, 2.5 Hz, | | |
| | 1H), 7.24 (d, J = 3.5 Hz, 1H), 7.11-7.18 (m, 3H), 4.41-4.73 (m, 2H), | | |
| | 4.12 (q, J = 6.9 Hz, 2H), 3.55-3.67 (m, 2H), 2.63-2.71 (m, 1H), | | |
| | 1.64-1.79 (m, 1H), 1.41-1.53 (m, 2H), 1.37 (t, J = 6.9 Hz, 3H), | | |
| | 1.05-1.23 (m, 13H), 0.87-1.00 (m, 3H), 0.70 (t, J = 6.9 Hz, 3H). | | |
| 69 | ¹H NMR (DMSO-d₆) (Rotamers present in NMR) d: 11.24 (br. s, | 1.23^{k} | 694.3 |
| | 2H), 8.93-9.03 (m, 1H), 8.53-8.68 (m, 1H), 8.02 (s, 1H), 7.67 (d, | | (M + H)⁺ |
| | J = 13.3 Hz, 1H), 7.61 (s, 1H), 7.25 (d, J = 3.5 Hz, 1H), 7.15 (d, J = 3.5 Hz, | | |
| | 1H), 7.15 (dd, J = 14.2, 1.9 Hz, 1H), 4.43-4.69 (m, 2H), 4.11 (q, | | |
| | J = 7.1 Hz, 2H), 3.80-3.91 (m, 1H), 2.54-2.62 (m, 1H), 2.35-2.43 (m, | | |
| | 2H), 1.57-1.70 (m, 1H), 1.41-1.57 (m, 4H), 1.37 (t, J = 6.9 Hz, 3H), | | |
| | 1.03-1.31 (m, 17H), 0.78-0.91 (m, 6H), 0.71 (t, J = 6.9 Hz, 3H). | | |
| 70 | ¹H NMR (DMSO-d₆) (Rotamers present in NMR) d: 12.79-13.21 (br. | 0.77^{k} | 836.3 |
| | s, 1H), 12.36-12.78 (br. s, 1H), 8.88 (d, J = 8.0 Hz, 2H), 8.16 (br. s, | | (M + H)⁺ |
| | 1H), 8.04-8.12 (m, 2H), 7.99 (d, J = 8.3 Hz, 1H), 7.53-7.65 (m, 3H), | | |
| | 7.20-7.27 (m, 2H), 7.11 (s, 1H), 6.98 (s, 1H), 4.76-4.88 (m, 1H), | | |
| | 4.52-4.74 (m, 2H), 4.36-4.51 (m, 1H), 4.29 (q, J = 7.4 Hz, 2H), | | |
| | 3.79-4.09 (m, 3H), 3.43-3.75 (m, 4H), 3.00-3.35 (m, 3H), 2.76-2.96 (m, | | |
| | 2H), 2.66-2.75 (m, 1H), 1.60-1.80 (m, 1H), 1.47 (t, J = 7.0 Hz, 3H), | | |
| | 1.40-1.58 (m, 2H), 1.08-1.28 (m, 7H), 0.96 (t, J = 7.2 Hz, 3H), 0.73 (t, | | |
| | J = 6.9 Hz, 3H). | | |
| 71 | ¹H NMR (DMSO-d₆) (Rotamers present in NMR) d: 12.92 (br. s, | 0.79^{k} | 850.4 |
| | 2H), 8.90 (d, J = 7.5 Hz, 2H), 8.75 (br. s, 1H), 8.14 (br. s, 1H), | | (M + H)⁺ |
| | 7.99 (d, J = 8.0 Hz, 1H), 7.86-7.95 (m, 1H), 7.54-7.64 (m, 2H), | | |
| | 7.15-7.30 (m, 4H), 4.51-4.81 (m, 3H), 4.28 (q, J = 6.9 Hz, 2H), 4.00 (br. s, 1H), | | |
| | 3.57 (t, J = 4.4 Hz, 3H), 3.41-3.48 (m, 2H), 2.57-2.93 (m, 4H), | | |
| | 2.43 (s, 3H), 2.27-2.37 (m, 4H), 1.63-1.78 (m, 1H), 1.47 (t, J = 6.9 Hz, 3H), | | |
| | 1.40-1.58 (m, 2H), 1.07-1.27 (m, 7H), 0.88-1.02 (m, 3H), 0.73 (t, | | |
| | J = 6.9 Hz, 3H). | | |
| 72 | ¹H NMR (DMSO-d₆) (Rotamers present in NMR) d: 12.96 (br. s, | 0.90^{k} | 718.3 |
| | 1H), 12.54 (br. s, 1H), 9.10-9.17 (m, J = 7.3 Hz, 0.25H), | | (M + H)⁺ |
| | 8.89-8.93 (m, 0.75H), 8.88 (d, J = 7.8 Hz, 1H), 8.39 (t, J = 5.0 Hz, 0.75H), | | |
| | 8.29-8.36 (m, 0.25H), 8.12 (s, 0.25H), 8.01 (s, 1H), 7.97-8.00 (m, 0.75H), | | |
| | 7.61-7.65 (m, 1H), 7.59-7.61 (m, 1H), 7.34-7.45 (m, 1H), 7.31 (d, | | |
| | J = 3.6 Hz, 1H), 7.27 (d, J = 3.5 Hz, 1H), 4.76-4.88 (m, 1H), | | |
| | 4.51-4.74 (m, 2H), 4.25-4.37 (m, 2H), 3.73-3.86 (m, 1H), 3.44-3.65 (m, 1H), | | |
| | 2.74-2.98 (m, 2H), 2.53-2.57 (m, 1H), 1.56-1.72 (m, 1H), 1.47 (t, | | |
| | J = 6.9 Hz, 3H), 1.29-1.57 (m, 3H), 1.08-1.22 (m, 7H), 0.96-1.07 (m, | | |
| | 5H), 0.90 (t, J = 6.7 Hz, 2.25H), 0.80-0.86 (m, 0.75H), 0.69-0.76 (m, | | |
| | 3H). | | |
| 73 | ¹H NMR (DMSO-d₆) (Rotamers present in NMR) d: 8.95-9.07 (m, | 0.80^{k} | 757.3 |
| | 1H), 8.66-8.92 (m, 1H), 8.12 (br. s, 1H), 7.99 (d, J = 6.8 Hz, 2H), | | (M + H)⁺ |
| | 7.67 (d, J = 13.1 Hz, 1H), 7.42-7.55 (m, 3H), 7.22 (d, J = 3.3 Hz, 1H), | | |
| | 7.15 (dd, J = 14.1, 1.8 Hz, 1H), 7.07 (d, J = 3.3 Hz, 1H), 4.63 (br. s, 1H), | | |
| | 4.48-4.59 (m, 1H), 4.06 (q, J = 6.9 Hz, 2H), 3.93-4.02 (m, 1H), | | |
| | 3.55-3.66 (m, 6H), 2.62-2.76 (m, 1H), 2.32-2.47 (m, 4H), 1.61-1.76 (m, | | |
| | 1H), 1.37-1.57 (m, 3H), 1.34 (t, J = 7.0 Hz, 3H), 1.06-1.27 (m, 6H), | | |
| | 0.95 (t, J = 7.0 Hz, 3H), 0.72 (t, J = 7.0 Hz, 3H). | | |
| 74 | ¹H NMR (MEOH-d₄) (Rotamers present in NMR) d: 9.07-9.17 (m, | 2.04^{b} | 633.3 |
| | 0.3H), 8.85 (t, J = 5.1 Hz, 0.7H), 8.57 (t, J = 6.0 Hz, 0.3H), 8.50 (t, | | (M − tBuNCO + 1) |
| | J = 6.0 Hz, 0.7H), 8.09-8.14 (m, 1H), 8.00-8.07 (m, 1H), | | |
| | 7.68-7.74 (m, 1H), 7.55 (dd, J = 8.2, 1.4 Hz, 1H), 7.37-7.49 (m, 1H), | | |
| | 7.22-7.31 (m, 1H), 7.10-7.16 (m, 1H), 6.82-6.99 (m, 1H), 4.98 (t, J = 4.5 Hz, | | |
| | 1H), 4.74-4.85 (m, 2H), 4.30-4.46 (m, 2H), 3.76-3.89 (m, 1H), | | |
| | 2.89-3.17 (m, 2H), 2.47-2.61 (m, 1H), 1.68-1.81 (m, 1H), 1.56-1.64 (m, | | |
| | 3H), 1.45-1.67 (m, 3H), 1.33 (s, 2H), 1.24-1.36 (m, 6H), 1.24 (s, | | |
| | 7H), 1.03 (t, J = 7.0 Hz, 2H), 0.96 (t, J = 7.2 Hz, 1H), 0.83 (t, J = 6.8 Hz, | | |
| | 3H). | | |
| 75 | ¹H NMR (MEOH-d₄) (Rotamers present in NMR) d: 9.28 (d, J = 7.8 Hz, | 2.15^{b} | 633.3 |
| | 1H), 8.99-9.07 (m, 1H), 8.73-8.83 (m, 1H), 8.37-8.46 (m, 1H), | | (M − Ph(H₃C)₂CNCO + 1) |
| | 8.11 (d, J = 8.3 Hz, 1H), 7.98-8.05 (m, 1H), 7.63-7.71 (m, 1H), | | |
| | 7.54 (d, J = 8.3 Hz, 1H), 7.31-7.40 (m, 2H), 7.08-7.30 (m, 5H), 4.99 (t, | | |
| | J = 4.5 Hz, 1H), 4.70-4.85 (m, 2H), 4.27-4.41 (m, 2H), 3.71-3.88 (m, | | |
| | 1H), 2.89-3.17 (m, 2H), 2.47-2.63 (m, 1H), 1.69-1.81 (m, 1H), | | |
| | 1.66 (d, J = 5.3 Hz, 2H), 1.58 (d, J = 5.3 Hz, 4H), 1.50-1.63 (m, 5H), | | |
| | 1.16-1.37 (m, 7H), 0.97 (t, J = 7.0 Hz, 2H), 0.86-0.91 (m, 1H), | | |
| | 0.81-0.86 (m, 3H). | | |
| 76 | ¹H NMR (DMSO-d₆) (Rotamers present in NMR) d: 12.95 (br. s, | 1.11^{k} | 806.4 |
| | 1H), 12.56 (br. s, 1H), 8.88 (d, J = 7.8 Hz, 1H), 8.66-8.83 (m, 1H), | | (M + H)⁺ |
| | 8.14 (s, 1H), 7.98 (d, J = 8.3 Hz, 1H), 7.57 (d, J = 8.3 Hz, 1H), 7.54 (s, | | |
| | 1H), 7.26-7.37 (m, 2H), 7.19-7.25 (m, 3H), 6.95 (d, J = 6.0 Hz, 1H), | | |
| | 6.78 (d, J = 7.3 Hz, 1H), 4.78-4.85 (m, 1H), 4.55-4.77 (m, 2H), | | |
| | 4.22-4.33 (m, 2H), 3.92-4.06 (m, 1H), 3.14-3.20 (m, 4H), 2.75-2.96 (m, | | |
| | 2H), 2.62-2.74 (m, 1H), 1.89-1.95 (m, 4H), 1.63-1.77 (m, 1H), | | |
| | 1.46 (t, J = 6.9 Hz, 3H), 1.33-1.61 (m, 2H), 1.08-1.28 (m, 7H), 0.97 (t, | | |
| | J = 7.3 Hz, 3H), 0.73 (t, J = 6.8 Hz, 3H). | | |
| 77 | ¹H NMR (DMSO-d₆) (Rotamers present in NMR) d: 12.94 (br. s, | 1.11^{k} | 824.4 |
| | 1H), 12.54 (br. s, 1H), 8.88 (d, J = 7.8 Hz, 2H), 8.57-8.72 (m, 1H), | | (M + H)⁺ |
| | 8.08 (s, 1H), 7.98 (d, J = 8.0 Hz, 1H), 7.48-7.64 (m, 4H), 7.22 (s, 2H), | | |
| | 6.49-6.59 (m, 1H), 4.78-4.86 (m, 1H), 4.55-4.78 (m, 2H), | | |
| | 4.21-4.31 (m, 2H), 3.87-3.98 (m, 1H), 3.37-3.43 (m, 4H), 2.75-2.96 (m, 2H), | | |
| | 2.66-2.75 (m, 1H), 1.84-1.94 (m, 4H), 1.60-1.72 (m, 1H), 1.46 (t, | | |
| | J = 7.0 Hz, 3H), 1.37-1.55 (m, 2H), 1.09-1.29 (m, 7H), 0.96 (t, J = 7.2 Hz, | | |
| | 3H), 0.75 (t, J = 6.8 Hz, 3H). | | |
| 78 | ¹H NMR (DMSO-d₆) (Rotamers present in NMR) d: 12.95 (br. s, | 7.33^{b} | 820.3 |
| | 1H), 12.56 (br. s, 1H), 8.88 (d, J = 7.8 Hz, 2H), 8.67-8.82 (m, 1H), | | (M + H)⁺ |
| | 8.13 (s, 1H), 7.95-8.03 (m, 3H), 7.75 (d, J = 8.5 Hz, 2H), 7.56 (d, | | |
| | J = 8.3 Hz, 1H), 7.53 (s, 1H), 7.19-7.23 (m, 2H), 4.82 (dt, J = 7.8, 4.7 Hz, | | |
| | 1H), 4.54-4.77 (m, 2H), 4.28 (q, J = 6.9 Hz, 2H), 3.97 (t, J = 9.8 Hz, | | |
| | 1H), 3.75-3.87 (m, 2H), 2.76-2.96 (m, 2H), 2.65-2.75 (m, 1H), | | |
| | 2.53-2.56 (m, 2H), 1.99-2.10 (m, 2H), 1.61-1.77 (m, 1H), 1.47 (t, | | |
| | J = 6.9 Hz, 3H), 1.40-1.57 (m, 2H), 1.09-1.27 (m, 7H), 0.96 (t, J = 7.0 Hz, | | |
| | 3H), 0.74 (t, J = 6.8 Hz, 3H). | | |
| 79 | ¹H NMR (DMSO-d₆) (Rotamers present in NMR) d: 12.77-13.14 (m, | 1.07^{k} | 802.4 |
| | 1H), 12.34-12.71 (m, 1H), 8.88 (d, J = 8.0 Hz, 3H), 8.15 (s, 1H), | | (M + H)⁺ |
| | 8.06 (d, J = 8.3 Hz, 2H), 7.97 (d, J = 8.3 Hz, 1H), 7.61-7.72 (m, 2H), | | |
| | 7.51-7.60 (m, 2H), 7.49 (t, J = 2.1 Hz, 2H), 7.16-7.26 (m, 2H), 6.34 (t, | | |
| | J = 2.3 Hz, 2H), 4.82 (dt, J = 8.0, 4.7 Hz, 1H), 4.54-4.77 (m, 2H), | | |
| | 4.22-4.33 (m, 2H), 4.01 (t, J = 9.2 Hz, 1H), 2.76-2.96 (m, 2H), | | |
| | 2.68-2.75 (m, 1H), 1.63-1.79 (m, 1H), 1.46 (t, J = 6.9 Hz, 3H), 1.35-1.56 (m, | | |
| | 2H), 1.07-1.29 (m, 7H), 0.97 (t, J = 7.3 Hz, 3H), 0.74 (t, J = 6.9 Hz, | | |
| | 3H). | | |
| 80 | ¹H NMR (DMSO-d₆) (Rotamers present in NMR): 9.82-9.88 (m, | 0.66^{k} | 743.3 |
| | J = 5.5 Hz, 0.7H), 9.74-9.81 (m, 0.7H), 9.61-9.70 (m, 0.3H), 9.05 (d, | | (M + H)⁺ |
| | J = 5.0 Hz, 1H), 8.93-9.01 (m, 0.3H), 8.47 (s, 0.3H), 8.02 (d, J = 8.3 Hz, | | |
| | 1H), 7.82 (s, 0.7H), 7.66 (s, 1H), 7.53-7.62 (m, 1H), 7.37 (d, | | |
| | J = 3.5 Hz, 0.3H), 7.30 (d, J = 3.5 Hz, 0.7H), 7.26 (d, J = 3.3 Hz, 0.3H), | | |
| | 7.24 (d, J = 3.5 Hz, 0.7H), 5.13-5.23 (m, 2H), 5.02-5.13 (m, 1H), | | |
| | 4.72-4.83 (m, 0.3H), 4.47-4.57 (m, 0.3H), 4.29-4.40 (m, 2H), | | |
| | 4.21-4.29 (m, 1.4H), 4.13-4.20 (m, 0.3H) 3.70-3.82 (m, 0.7H), 2.94 (q, | | |
| | J = 8.2 Hz, 2H), 2.59-2.77 (m, 1H), 1.55-1.63 (m, 2H), 1.47-1.55 (m, | | |
| | 3H), 1.30-1.46 (m, 2H), 1.07-1.29 (m, 6H), 0.71-0.84 (m, 6H). | | |
| 81 | ¹H NMR (DMSO-d₆) (Rotamers present in NMR) d: 11.29 (br. s, | 6.94^{a} | 716.3 |
| | 2H), 9.02 (t, J = 5.8 Hz, 1H), 8.71-8.97 (m, 1H), 8.14-8.25 (m, 1H), | | (M + H)⁺ |
| | 8.08 (br. s, 1H), 7.66 (d, J = 13.3 Hz, 1H), 7.49-7.61 (m, 2H), | | |
| | 7.21-7.25 (m, 1H), 7.15 (dd, J = 14.7, 2.1 Hz, 1H), 7.12 (d, J = 3.5 Hz, 1H), | | |
| | 6.94-6.99 (m, 1H), 4.45-4.75 (m, 2H), 4.24-4.37 (m, 4H), 4.11 (q, | | |
| | J = 6.9 Hz, 2H), 3.55-3.67 (m, 1H), 2.65-2.79 (m, 1H), 1.60-1.74 (m, | | |
| | 1H), 1.41-1.55 (m, 2H), 1.36 (t, J = 7.0 Hz, 3H), 1.05-1.23 (m, 7H), | | |
| | 0.94 (t, J = 7.2 Hz, 3H), 0.70 (t, J = 7.0 Hz, 3H). | | |
| 82 | ¹H NMR (DMSO-d₆) (Rotamers present in NMR) d: 11.27 (br. s, | 0.96^{k} | 654.2 |
| | 2H), 9.04 (t, J = 6.0 Hz, 1H), 8.57-8.66 (m, 1H), 8.02 (s, 1H), 7.67 (d, | | (M + H)⁺ |
| | J = 13.3 Hz, 1H), 7.61 (s, 1H), 7.25 (d, J = 3.8 Hz, 1H), 7.12-7.19 (m, | | |
| | 2H), 4.44-4.71 (m, 2H), 4.12 (q, J = 6.9 Hz, 2H), 3.91 (t, J = 9.0 Hz, | | |
| | 1H), 2.55-2.65 (m, 1H), 1.58-1.72 (m, 1H), 1.45-1.47 (m, 1H), | | |
| | 1.43 (s, 9H), 1.34-1.40 (m, 4H), 1.06-1.21 (m, 7H), 0.89 (t, J = 6.5 Hz, 3H), | | |
| | 0.71 (t, J = 6.9 Hz, 3H). | | |
| 83 | ¹H NMR (DMSO-d₆) (Rotamers present in NMR) d: 9.04 (d, J = 5.3 Hz, | 7.60^{a} | 795.4 |
| | 1H), 8.74-8.96 (m, 1H), 8.10 (s, 1H), 8.01 (d, J = 8.0 Hz, 1H), | | (M + H)⁺ |
| | 7.44-7.61 (m, 4H), 7.19-7.28 (m, 2H), 6.93-7.15 (m, 1H), 6.91 (d, | | |
| | J = 8.5 Hz, 1H), 4.50-4.79 (m, 2H), 4.20-4.35 (m, 7H), 3.91-4.02 (m, | | |
| | 1H), 2.57-2.76 (m, 3H), 1.61-1.75 (m, 1H), 1.38-1.57 (m, 5H), | | |
| | 1.06-1.27 (m, 7H), 0.95 (t, J = 7.3 Hz, 3H), 0.73 (t, J = 6.9 Hz, 3H). | | |
| 84 | ¹H NMR (DMSO-d₆) (Rotamers present in NMR) d: 8.97-9.07 (m, | 7.69^{a} | 729.4 |
| | 1H), 8.75-8.96 (m, 1H), 8.10 (s, 1H), 7.68 (d, J = 12.0 Hz, 1H), | | (M + H)⁺ |
| | 7.42 (br. s, 2H), 7.18 (d, J = 3.3 Hz, 2H), 7.12-7.18 (m, 1H), 6.96 (d, J = 3.5 Hz, | | |
| | 1H), 6.76 (d, J = 8.3 Hz, 1H), 4.46-4.77 (m, 2H), 4.30 (t, J = 4.1 Hz, | | |
| | 2H), 3.90-4.04 (m, 3H), 3.20-3.29 (m, 2H), 2.85 (s, 3H), | | |
| | 2.58-2.72 (m, 1H), 1.58-1.72 (m, 1H), 1.35-1.54 (m, 2H), 1.30 (t, J = 7.0 Hz, | | |
| | 3H), 1.05-1.21 (m, 7H), 0.95 (t, J = 7.2 Hz, 3H), 0.69 (t, J = 6.9 Hz, | | |
| | 3H). | | |
| 85 | ¹H NMR (DMSO-d₆) (Rotamers present in NMR) d: 9.03 (d, J = 5.3 Hz, | 7.69^{a} | 808.4 |
| | 1H), 8.68-8.88 (m, 1H), 8.11 (s, 1H), 8.01 (d, J = 8.0 Hz, 1H), | | (M + H)⁺ |
| | 7.51-7.58 (m, 2H), 7.35-7.43 (m, 1H), 7.19-7.25 (m, 2H), | | |
| | 6.98-7.14 (m, 2H), 6.68 (d, J = 8.3 Hz, 1H), 4.53-4.79 (m, 2H), 4.22-4.37 (m, | | |
| | 5H), 3.91-4.02 (m, 1H), 3.19-3.26 (m, 2H), 2.77 (s, 3H), | | |
| | 2.56-2.71 (m, 3H), 1.61-1.74 (m, 1H), 1.41-1.55 (m, 5H), 1.06-1.27 (m, 7H), | | |
| | 0.96 (t, J = 7.2 Hz, 3H), 0.73 (t, J = 6.9 Hz, 3H). | | |
| 86 | ¹H NMR (DMSO-d₆) (Rotamers present in NMR) d: 12.96 (br. s, | 0.99^{k} | 733.4 |
| | 1H), 12.54 (br. s, 1H), 8.93-9.01 (m, 1H), 8.84-8.92 (m, 1H), | | (M + H)⁺ |
| | 8.59-8.69 (m, 1H), 7.98-8.05 (m, 2H), 7.59-7.66 (m, 2H), 7.29 (dd, | | |
| | J = 16.7, 3.6 Hz, 2H), 4.74-4.86 (m, 1H), 4.46-4.75 (m, 2H), 4.31 (q, | | |
| | J = 6.7 Hz, 2H), 3.85-3.96 (m, 1H), 2.71-3.00 (m, 2H), 2.57-2.64 (m, | | |
| | 1H), 1.59-1.73 (m, 1H), 1.43-1.52 (m, 2H), 1.41 (s, 9H), | | |
| | 1.08-1.21 (m, 7H), 0.78-0.93 (m, 6H), 0.72 (t, J = 7.0 Hz, 3H). | | |
| 87 | ¹H NMR (DMSO-d₆) (Rotamers present in NMR) d: 12.27-13.97 (m, | 1.10^{k} | 811.3 |
| | 3), 9.12 (d, J = 7.5 Hz, 1H), 8.83-8.97 (m, 1H), 8.47-8.69 (m, 1H), | | (M + H)⁺ |
| | 8.12 (s, 1H), 7.99 (d, J = 8.3 Hz, 1H), 7.68 (d, J = 9.5 Hz, 1H), 7.60 (s, | | |
| | 1H), 7.37 (t, J = 8.0 Hz, 1H), 7.22-7.32 (m, 2H), 6.83-7.02 (m, 2H), | | |
| | 4.98 (s, 2H), 4.81 (s, 1H), 4.43-4.74 (m, 2H), 3.96-4.18 (m, 1H), | | |
| | 3.81 (s, 3H), 2.72-2.89 (m, 2H), 2.55-2.69 (m, 1H), 2.27 (s, 3H), | | |
| | 1.64-1.81 (m, 1H), 1.52-1.64 (m, 1H), 1.47 (s, 2H), 1.06-1.22 (m, | | |
| | 6H), 0.88-1.02 (m, 3H), 0.70 (t, J = 6.9 Hz, 3H). | | |
| 88 | ¹H NMR (DMSO-d₆) (Rotamers present in NMR) d: 9.01-9.12 (m, | 7.85^{a} | 702.3 |
| | 1H), 8.69-9.00 (m, 1H), 8.10 (br. s, 1H), 7.88-8.05 (m, 1H), 7.69 (d, | | (M + H)⁺ |
| | J = 12.3 Hz, 1H), 7.41 (s, 1H), 7.18 (d, J = 3.5 Hz, 1H), 7.15 (dd, | | |
| | J = 13.1, 2.3 Hz, 1H), 6.96 (d, J = 3.5 Hz, 1H), 6.90 (d, J = 2.3 Hz, 1H), | | |
| | 6.82-6.88 (m, 1H), 4.47-4.75 (m, 2H), 3.92-4.05 (m, 3H), 3.79 (s, | | |
| | 3H), 2.56-2.70 (m, 1H), 2.48 (s, 3H), 1.59-1.74 (m, 1H), | | |
| | 1.34-1.55 (m, 2H), 1.30 (t, J = 7.0 Hz, 3H), 1.03-1.24 (m, 7H), 0.86-1.01 (m, | | |
| | 3H), 0.70 (t, J = 6.9 Hz, 3H). | | |
| 89 | ¹H NMR (DMSO-d₆) (Rotamers present in NMR) d: 9.02-9.12 (m, | 1.11^{k} | 730.3 |
| | J = 6.0, 6.0 Hz, 1H), 8.55-8.79 (m, 1H), 8.11 (br. s, 1H), | | (M + H)⁺ |
| | 7.80-7.94 (m, 1H), 7.66 (d, J = 13.3 Hz, 1H), 7.61 (s, 1H), 7.25 (d, J = 3.5 Hz, | | |
| | 1H), 7.11-7.19 (m, 2H), 6.97 (d, J = 2.5 Hz, 1H), 6.83 (d, J = 5.8 Hz, | | |
| | 1H), 4.48-4.72 (m, 2H), 4.12 (q, J = 6.9 Hz, 2H), 3.92-4.04 (m, 1H), | | |
| | 3.81 (s, 3H), 3.58-3.77 (m, 1H), 2.57-2.68 (m, 1H), 1.62-1.79 (m, | | |
| | 1H), 1.40-1.52 (m, 2H), 1.37 (t, J = 6.9 Hz, 3H), 1.06-1.22 (m, 13H), | | |
| | 0.89-1.01 (m, 3H), 0.70 (t, J = 7.0 Hz, 3H). | | |
| 90 | ¹H NMR (DMSO-d₆) (Rotamers present in NMR) d: 12.81 (s, 3H), | 1.97^{b} | 782.2 |
| | 10.06 (s, 0.2H), 10.01 (br. s, 0.8H), 9.19 (t, J = 5.5 Hz, 0.2H), 9.11 (d, | | (M + H)⁺ |
| | J = 7.8 Hz, 1H), 9.04 (t, J = 5.5 Hz, 0.8H), 8.49 (t, J = 5.1 Hz, 0.8H), | | |
| | 8.42-8.46 (m, 0.2H), 8.28 (s, 0.2H), 8.11 (s, 0.8H), 7.98 (d, J = 8.0 Hz, | | |
| | 1H), 7.65 (d, J = 8.3 Hz, 1H), 7.57 (s, 1H), 7.47 (d, J = 8.0 Hz, | | |
| | 0.4H), 7.42 (d, J = 7.8 Hz, 1.6H), 7.24-7.37 (m, 4H), 7.06-7.12 (m, | | |
| | 0.2H), 6.99-7.06 (m, 0.8H), 4.98 (s, 2H), 4.77-4.87 (m, 1H), | | |
| | 4.52-4.75 (m, 2H), 4.34 (t, J = 9.3 Hz, 0.2H), 3.95 (t, J = 9.3 Hz, 0.8H), | | |
| | 2.72-2.91 (m, 2H), 2.58-2.69 (m, 1H), 1.62-1.77 (m, 1H), | | |
| | 1.31-1.59 (m, 3H), 1.03-1.29 (m, 6H), 0.93 (t, J = 6.5 Hz, 2.4H), 0.87 (t, J = 6.5 Hz, | | |
| | 0.6H), 0.71 (t, J = 6.7 Hz, 3H). | | |
| 91 | ¹H NMR (MEOH-d₄) (Rotamers present in NMR) d: 9.27 (d, J = 7.8 Hz, | 6.77^{a} | 797.3 |
| | 1H), 8.84-8.99 (m, 1H), 8.47-8.60 (m, 1H), 8.20 (s, 1H), | | (M + H)⁺ |
| | 8.01-8.07 (m, 1H), 7.65 (dd, J = 8.5, 2.0 Hz, 1H), 7.33-7.45 (m, 2H), | | |
| | 7.21-7.27 (m, 1H), 7.19 (d, J = 3.5 Hz, 1H), 6.92-6.99 (m, 1H), | | |
| | 6.61-6.71 (m, 1H), 4.96-5.03 (m, 1H), 4.83-4.90 (m, 2H), 4.28-4.42 (m, 1H), | | |
| | 4.25 (q, J = 7.0 Hz, 2H), 3.76 (s, 3H), 3.62 (s, 3H), 2.90-3.18 (m, 2H), | | |
| | 2.59-2.71 (m, 1H), 1.74-1.88 (m, 1H), 1.62-1.74 (m, 1H), 1.55 (t, | | |
| | J = 6.9 Hz, 3H), 1.45-1.62 (m, 2H), 1.22-1.41 (m, 6H), 1.00-1.14 (m, | | |
| | 3H), 0.89 (t, J = 5.8 Hz, 3H). | | |
| 92 | ¹H NMR (DMSO-d₆) (Rotamers present in NMR) d: 10.06-10.10 (m, | 7.49^{a} | 673.2 |
| | 0.2H), 10.03 (br. s, 0.8H), 9.15-9.21 (m, 0.2H), 9.11 (t, J = 5.6 Hz, | | (M + H)⁺ |
| | 0.8H), 8.48 (t, J = 5.8 Hz, 1H), 8.30 (s, 0.2H), 8.10 (s, 0.8H), 7.67 (d, | | |
| | J = 13.3 Hz, 1H), 7.59 (s, 1H), 7.42-7.51 (m, 2H), 7.26-7.36 (m, 2H), | | |
| | 7.21-7.26 (m, 1H), 7.11-7.19 (m, 2H), 6.95-7.10 (m, 1H), | | |
| | 4.50-4.72 (m, 2H), 4.34 (t, J = 9.0 Hz, 0.2H), 4.10 (q, J = 6.9 Hz, 2H), 3.94 (t, | | |
| | J = 9.3 Hz, 0.8H), 2.56-2.69 (m, 1H), 1.60-1.75 (m, 1H), | | |
| | 1.40-1.56 (m, 2H), 1.36 (t, J = 6.9 Hz, 3H), 1.03-1.23 (m, 7H), 0.93 (t, J = 6.8 Hz, | | |
| | 2.4H), 0.84-0.89 (m, 0.6H), 0.69 (t, J = 6.8 Hz, 3H). | | |
| 93 | ¹H NMR (MEOH-d₄) (Rotamers present in NMR) d: 9.25 (d, J = 7.8 Hz, | 1.04^{k} | 751.3 |
| | 1H), 8.80-9.00 (m, 1H), 8.74 (t, J = 6.4 Hz, 1H), 8.20 (s, 1H), | | (M + H)⁺ |
| | 8.06 (d, J = 8.3 Hz, 1H), 7.95 (d, J = 8.0 Hz, 1H), 7.55 (s, 1H), | | |
| | 7.41-7.47 (m, 1H), 7.27-7.40 (m, 1H), 7.08-7.23 (m, 3H), 7.01 (d, J = 3.3 Hz, | | |
| | 1H), 4.96-5.02 (m, 1H), 4.84-4.90 (m, 2H), 4.23-4.35 (m, 2H), | | |
| | 3.93-4.12 (m, 1H), 2.88-3.20 (m, 2H), 2.54-2.70 (m, 1H), 2.42 (s, 3H), | | |
| | 1.74-1.91 (m, 1H), 1.56 (t, J = 7.0 Hz, 3H), 1.47-1.71 (m, 3H), | | |
| | 1.19-1.39 (m, 6H), 1.06-1.15 (m, 3H), 0.82-0.93 (m, 3H). | | |
| 94 | ¹H NMR (MEOH-d₄) (Rotamers present in NMR) d: 9.28 (d, J = 8.0 Hz, | 7.09^{a} | 747.3 |
| | 1H), 9.07-9.15 (m, 0.5H), 8.96 (t, J = 6.3 Hz, 0.5H), 8.73-8.83 (m, | | (M + H)⁺ |
| | 1H), 8.32 (s, 0.5H), 8.12 (d, J = 8.3 Hz, 1H), 8.02 (s, 0.5H), 7.71 (s, | | |
| | 1H), 7.53-7.61 (m, 1H), 7.28 (t, J = 3.4 Hz, 1H), 7.10-7.16 (m, 1H), | | |
| | 5.36-5.62 (m, 2H), 4.95-5.05 (m, 1H), 4.68-4.85 (m, 2H), 4.40 (q, | | |
| | J = 6.9 Hz, 2H), 4.17-4.27 (m, 0.5H), 3.73 (t, J = 9.3 Hz, 0.5H), | | |
| | 2.88-3.17 (m, 2H), 2.60-2.79 (m, 1H), 1.74-1.88 (m, 1H), 1.64-1.72 (m, | | |
| | 1H), 1.61 (t, J = 7.0 Hz, 3H), 1.52-1.65 (m, 2H), 1.22-1.34 (m, 6H), | | |
| | 1.20 (s, 4.5H), 1.13 (s, 4.5H), 1.00 (t, J = 7.0 Hz, 1.5H), 0.93 (t, J = 7.2 Hz, | | |
| | 1.5H), 0.76-0.84 (m, 3H). | | |
| 95 | ¹H NMR (DMSO-d₆) (Rotamers present in NMR) d: 12.92 (br. s, | 0.99^{k} | 820.3 |
| | 3H), 9.13 (d, J = 7.8 Hz, 1H), 8.88-9.03 (m, 1H), 8.55 (t, J = 5.4 Hz, | | (M + H)⁺ |
| | 1H), 8.06-8.14 (m, 1H), 8.00 (d, J = 8.3 Hz, 1H), 7.68 (d, J = 8.8 Hz, | | |
| | 1H), 7.59 (s, 1H), 7.29 (dd, J = 16.9, 3.6 Hz, 2H), 4.97 (s, 2H), | | |
| | 4.81 (q, J = 7.0 Hz, 1H), 4.48-4.71 (m, 2H), 3.84-4.00 (m, 1H), | | |
| | 3.08-3.13 (m, 3H), 2.72-2.90 (m, 2H), 2.53-2.64 (m, 1H), 1.57-1.73 (m, 1H), | | |
| | 1.43 (s, 9H), 1.32-1.57 (m, 2H), 1.07-1.30 (m, 7H), 0.78-0.98 (m, | | |
| | 3H), 0.72 (t, J = 6.7 Hz, 3H). | | |
| 96 | ¹H NMR (MEOH-d₄) (Rotamers present in NMR) d: 8.62-8.80 (m, | 6.87^{a} | 797.3 |
| | 1H), 8.18 (s, 1H), 8.07 (d, J = 8.3 Hz, 1H), 7.85 (d, J = 8.8 Hz, 1H), | | (M + H)⁺ |
| | 7.37-7.54 (m, 3H), 7.22 (d, J = 3.5 Hz, 1H), 7.02 (d, J = 2.8 Hz, 1H), | | |
| | 6.60-6.74 (m, 1H), 6.09-6.37 (m, 2H), 5.00 (t, J = 4.6 Hz, 1H), | | |
| | 4.94-4.97 (m, 2H), 4.24 (q, J = 6.9 Hz, 2H), 3.85-4.01 (m, 1H), 3.70 (s, | | |
| | 3H), 3.68 (s, 3H), 2.88-3.17 (m, 2H), 2.55-2.67 (m, 1H), | | |
| | 1.74-1.88 (m, 1H), 1.55 (t, J = 6.9 Hz, 3H), 1.50-1.73 (m, 3H), 1.21-1.40 (m, | | |
| | 6H), 0.97-1.16 (m, 3H), 0.83-0.96 (m, 3H). | | |
| 97 | ¹H NMR (DMSO-d₆) (Rotamers present in NMR) d: 9.03 (t, J = 6.0 Hz, | 7.68^{a} | 757.3 |
| | 1H), 8.49-8.90 (m, 1H), 8.01-8.14 (m, 1H), 7.80-7.96 (m, 1H), | | (M + H)⁺ |
| | 7.65 (d, J = 12.8 Hz, 1H), 7.51 (s, 1H), 7.21 (d, J = 3.5 Hz, 1H), | | |
| | 7.15 (dd, J = 13.9, 1.9 Hz, 1H), 7.03 (d, J = 3.3 Hz, 1H), 6.83 (d, J = 1.8 Hz, | | |
| | 1H), 6.68-6.78 (m, J = 9.5 Hz, 1H), 4.48-4.74 (m, 2H), 4.02 (q, J = 6.8 Hz, | | |
| | 2H), 3.89-3.97 (m, 1H), 3.63-3.75 (m, 4H), 3.18-3.28 (m, 4H), | | |
| | 2.55-2.72 (m, 1H), 2.44 (s, 3H), 1.55-1.74 (m, 1H), 1.36-1.54 (m, | | |
| | 2H), 1.31 (t, J = 6.9 Hz, 3H), 1.04-1.24 (m, 7H), 0.95 (t, J = 6.7 Hz, | | |
| | 3H), 0.71 (t, J = 7.0 Hz, 3H). | | |
| 98 | ¹H NMR (MEOH-d₄) (Rotamers present in NMR) d: 9.26 (d, J = 7.8 Hz, | 7.61^{a} | 779.2 |
| | 1H), 8.81-8.97 (m, 1H), 8.76 (t, J = 5.8 Hz, 1H), 8.21 (s, 1H), | | (M + H)⁺ |
| | 8.07 (d, J = 8.3 Hz, 1H), 7.82 (dd, J = 7.8, 1.0 Hz, 1H), 7.63 (d, J = 1.3 Hz, | | |
| | 1H), 7.37-7.55 (m, 3H), 7.25 (d, J = 3.8 Hz, 1H), 7.09-7.20 (m, 1H), | | |
| | 7.07 (d, J = 3.8 Hz, 1H), 4.95-5.02 (m, 1H), 4.77-4.89 (m, 2H), | | |
| | 4.25-4.39 (m, 2H), 3.93-4.13 (m, 1H), 3.54-3.68 (m, 1H), 2.90-3.15 (m, | | |
| | 2H), 2.56-2.69 (m, 1H), 1.77-1.93 (m, 1H), 1.57 (t, J = 6.9 Hz, 3H), | | |
| | 1.46-1.70 (m, 2H), 1.20-1.40 (m, 7H), 1.13-1.18 (m, 6H), | | |
| | 1.02-1.13 (m, 3H), 0.84 (t, J = 6.9 Hz, 3H). | | |
| 99 | ¹H NMR (MEOH-d₄) (Rotamers present in NMR) d: 9.20-9.33 (m, | 5.96^{a} | 766.3 |
| | 1H), 8.75-8.93 (m, 2H), 8.52 (d, J = 5.8 Hz, 1H), 8.23 (s, 1H), 8.10 (d, | | (M + H)⁺ |
| | J = 8.3 Hz, 1H), 7.69 (s, 1H), 7.50-7.58 (m, 2H), 7.23 (d, J = 3.5 Hz, | | |
| | 1H), 7.12 (d, J = 3.5 Hz, 1H), 5.00 (t, J = 4.5 Hz, 1H), 4.78 (t, J = 5.4 Hz, | | |
| | 2H), 4.33-4.43 (m, 2H), 4.04-4.18 (m, 1H), 2.89-3.20 (m, 2H), | | |
| | 2.70 (s, 3H), 2.58-2.63 (m, 1H), 2.56 (s, 3H), 1.83-1.98 (m, 1H), | | |
| | 1.60 (t, J = 6.9 Hz, 3H), 1.46-1.70 (m, 2H), 1.19-1.36 (m, 7H), | | |
| | 1.04-1.15 (m, 3H), 0.80-0.86 (m, 3H). | | |
| 100 | 1H NMR (DMSO-d6) (Rotamers present in NMR) d: 9.68 (s, 0.2H), | 1.05^{k} | 502.4 |
| | 9.31 (br. s, 0.6H), 9.23-9.29 (m, 0.3H), 9.11 (t, J = 5.9 Hz, 0.6H), | | (M + H)⁺ |
| | 8.83 (br. s, 0.2H), 8.56 (br. s, 0.6H), 8.29 (s, 0.3H), 8.05 (q, J = 8.6 Hz, | | |
| | 4H), 7.76 (s, 0.7H), 7.28-7.32 (m, 2H), 4.49-4.72 (m, 2H), 4.34 (q, | | |
| | J = 7.2 Hz, 2H), 4.18-4.25 (m, 0.3H), 3.54-3.61 (m, 0.7H), | | |
| | 2.53-2.63 (m, 1H), 1.31-1.54 (m, 7H), 1.06-1.26 (m, 6H), 0.65-0.83 (m, 6H). | | |
| 101 | 1H NMR (MEOH-d4) (Rotamers present in NMR) d: 8.32 (s, 0.3H), | 1.17^{k} | 530.2 |
| | 7.97-8.12 (m, 4H), 7.87 (s, 0.8H), 7.29 (d, J = 3.5 Hz, 1H), | | (M + H)⁺ |
| | 7.09-7.14 (m, 1H), 4.70-4.90 (m, 2H), 4.37 (t, J = 6.5 Hz, 2.4H), 3.64 (d, J = 9.3 Hz, | | |
| | 0.8H), 2.72 (d, J = 4.8 Hz, 1H), 1.43-1.91 (m, 8H), 1.15-1.37 (m, | | |
| | 6H), 1.03 (t, J = 7.4 Hz, 3H), 0.84-0.95 (m, 3H), 0.73-0.83 (m, 3H). | | |
| 102 | 1H NMR (DMSO-d6) (Rotamers present in NMR) d: 9.62 (s, 0.2H), | 1.18^{k} | 578.2 |
| | 9.30 (s, 0.6H), 9.27 (s, 0.3H), 9.12 (t, J = 6.0 Hz, 0.6H), 8.80 (s, | | (M + H)⁺ |
| | 0.2H), 8.55 (t, J = 5.6 Hz, 0.6H), 8.29 (s, 0.3H), 8.02-8.11 (m, 2H), | | |
| | 7.92-8.01 (m, 2H), 7.76 (s, 0.7H), 7.32-7.36 (m, 4H), 7.27-7.32 (m, | | |
| | 2H), 7.20-7.27 (m, 1H), 4.52-4.75 (m, 2H), 4.50 (t, J = 6.7 Hz, 2H), | | |
| | 4.22 (d, J = 7.8 Hz, 0.3H), 3.53-3.61 (m, 0.7H), 3.06 (t, J = 6.7 Hz, | | |
| | 2H), 2.53-2.63 (m, 1H), 1.34-1.56 (m, 4H), 1.06-1.24 (m, 6H), | | |
| | 0.65-0.81 (m, 6H). | | |
| 103 | 1H NMR (DMSO-d6) (Rotamers present in NMR) d: 9.61 (s, 0.2H), | 0.69^{k} | 587.2 |
| | 9.30 (s, 0.6H), 9.26 (t, J = 6.0 Hz, 0.3H), 9.11 (t, J = 6.0 Hz, 0.6H), | | (M + H)⁺ |
| | 8.80 (s, 0.2H), 8.55 (t, J = 5.9 Hz, 0.7H), 8.29 (s, 0.3H), 7.99-8.11 (m, | | |
| | 4H), 7.76 (s, 0.7H), 7.28-7.31 (m, 2H), 4.49-4.72 (m, 2H), 4.41 (t, | | |
| | J = 5.8 Hz, 2H), 4.22 (d, J = 7.5 Hz, 0.3H), 3.52-3.61 (m, 4.6H), | | |
| | 2.71 (t, J = 5.8 Hz, 2H), 2.54-2.64 (m, 1H), 2.33-2.49 (m, 4H), | | |
| | 1.34-1.56 (m, 4H), 1.05-1.23 (m, 6H), 0.65-0.81 (m, 6H). | | |
| 104 | 1H NMR (MEOH-d4) (Rotamers present in NMR) d: 8.32 (s, 0.3H), | 0.67^{k} | 545.2 |
| | 8.11-8.18 (m, 2H), 7.98-8.04 (m, 2H), 7.87 (s, 0.7H), 7.29 (d, J = 3.8 Hz, | | (M + H)⁺ |
| | 1H), 7.11-7.15 (m, 1H), 4.71-4.88 (m, 2H), 4.49 (t, J = 5.5 Hz, | | |
| | 2H), 4.28-4.40 (m, 0.3H), 3.59-3.68 (m, 0.7H), 2.82 (t, J = 5.5 Hz, | | |
| | 2H), 2.72 (td, J = 9.8, 4.8 Hz, 0.7H), 2.54-2.67 (m, 0.3H), 2.39 (s, | | |
| | 6H), 1.49-1.74 (m, 4H), 1.15-1.33 (m, 6H), 0.85-0.98 (m, 3H), | | |
| | 0.68-0.83 (m, 3H). | | |
| 105 | 1H NMR (DMSO-d6) (Rotamers present in NMR) d: 9.61 (s, 0.3H), | 0.84^{k} | 559.2 |
| | 9.30 (s, 0.7H), 9.28 (s, 0.2H), 9.13 (t, J = 6.0 Hz, 0.7H), 8.80 (s, | | (M + H)⁺ |
| | 0.3H), 8.55 (t, J = 5.8 Hz, 0.7H), 8.29 (s, 0.3H), 8.08 (q, J = 8.8 Hz, | | |
| | 4H), 7.76 (s, 0.7H), 7.29-7.33 (m, 2H), 5.06 (s, 2H), 4.49-4.72 (m, | | |
| | 2H), 4.22 (d, J = 7.8 Hz, 0.3H), 3.54-3.61 (m, 0.7H), 3.00 (s, 3H), | | |
| | 2.85 (s, 3H), 2.53-2.63 (m, 1H), 1.34-1.56 (m, 4H), 1.05-1.23 (m, | | |
| | 6H), 0.65-0.81 (m, 6H). | | |
| 106 | ¹H NMR (DMSO-d₆) (Rotamers present in NMR) d: 12.68 (br. s, | 0.97^{k} | 733.6 |
| | 1H), 9.60 (s, 1H), 9.17 (t, J = 5.9 Hz, 0.3H), 9.03 (t, J = 6.0 Hz, 0.7H), | | (M + H)⁺ |
| | 8.88 (d, J = 7.8 Hz, 1H), 8.77 (t, J = 5.6 Hz, 0.3H), 8.48-8.57 (m, | | |
| | 0.7H), 8.29 (s, 0.3H), 7.97 (d, J = 8.0 Hz, 1H), 7.77 (s, 0.7H), | | |
| | 7.58-7.67 (m, 2H), 7.25-7.36 (m, 2H), 5.72-5.80 (m, 2H), 4.97 (dt, J = 8.0, | | |
| | 5.0 Hz, 0.7H), 4.82-4.90 (m, 0.3H), 4.50-4.76 (m, 2H), 4.27-4.38 (m, | | |
| | 2H), 4.16-4.26 (m, 0.3H), 3.48-3.61 (m, 0.7H), 2.88 (qd, J = 17.2, 5.0 Hz, | | |
| | 2H), 2.55-2.64 (m, 2H), 1.44-1.55 (m, 5H), 1.31-1.42 (m, 2H), | | |
| | 1.11-1.27 (m, 6H), 1.09 (d, J = 7.0 Hz, 6H), 0.65-0.83 (m, 6H). | | |
| 107 | ¹H NMR (DMSO-d₆) (Rotamers present in NMR) d: 9.62 (br. s, | 8.61^{a} | 786.3 |
| | 0.3H), 9.57 (s, 0.7H), 9.25-9.29 (m, 0.3H), 9.15 (t, J = 6.0 Hz, 0.7H), | | (M + H)⁺ |
| | 8.79 (t, J = 5.6 Hz, 0.3H), 8.53 (t, J = 5.6 Hz, 0.7H), 8.29 (s, 0.3H), | | |
| | 7.80 (s, 1H), 7.76 (s, 0.7H), 7.73 (d, J = 14.8 Hz, 1H), 7.27 (s, 0.3H), | | |
| | 7.26 (s, 0.7H), 7.14 (d, J = 15.6 Hz, 0.7H), 7.06 (d, J = 15.8 Hz, 0.3H), | | |
| | 5.64-5.77 (m, 4H), 4.36-4.84 (m, 4H), 4.05-4.26 (m, 2H), | | |
| | 3.49-3.64 (m, J = 5.0 Hz, 1H), 2.54-2.65 (m, 1H), 1.45-1.60 (m, 2H), | | |
| | 1.28-1.45 (m, 5H), 1.02-1.27 (m, 19H), 0.65-0.83 (m, 6H). | | |
| 108 | ¹H NMR (DMSO-d₆) (Rotamers present in NMR) d: 9.64 (br. s, | 9.04^{a} | 706.3 |
| | 0.3H), 9.30 (s, 0.7H), 9.25-9.29 (m, 0.3H), 9.16 (t, J = 6.0 Hz, 0.7H), | | (M + H)⁺ |
| | 8.79 (t, J = 5.6 Hz, 0.3H), 8.54 (t, J = 5.1 Hz, 0.7H), 8.29 (s, 0.3H), | | |
| | 7.96 (d, J = 14.3 Hz, 1H), 7.82 (s, 1H), 7.76 (s, 0.7H), 7.29-7.44 (m, | | |
| | 6H), 7.17-7.29 (m, 7H), 4.46-4.74 (m, 2H), 4.08-4.26 (m, 2.3H), | | |
| | 3.57 (td, J = 9.3, 4.9 Hz, 0.7H), 2.53-2.64 (m, 1H), 1.43-1.56 (m, 2H), | | |
| | 1.30-1.42 (m, 5H), 1.05-1.19 (m, 6H), 0.72-0.80 (m, 3H), | | |
| | 0.62-0.71 (m, 3H). | | |
| 109 | ¹H NMR (DMSO-d₆) (Rotamers present in NMR) d: 9.61 (s, 0.3H), | 8.66^{a} | 754.3 |
| | 9.29 (s, 0.7H), 9.24-9.28 (m, 0.3H), 9.14 (t, J = 6.0 Hz, 0.7H), | | (M + H)⁺ |
| | 8.75-8.83 (m, 0.3H), 8.53 (t, J = 5.8 Hz, 0.7H), 8.29 (s, 0.3H), 7.79 (s, 1H), | | |
| | 7.76 (s, 0.7H), 7.72 (d, J = 14.6 Hz, 1H), 7.27 (s, 0.6H), 7.26 (s, | | |
| | 1.4H), 7.12 (d, J = 15.3 Hz, 1H), 5.64-5.77 (m, 4H), 4.45-4.74 (m, | | |
| | 2H), 4.07-4.26 (m, 2.3H), 3.58 (td, J = 9.5, 4.8 Hz, 0.7H), | | |
| | 2.54-2.64 (m, 1H), 2.41-2.49 (m, 2H), 1.45-1.57 (m, 2H), 1.33-1.43 (m, 5H), | | |
| | 1.05-1.22 (m, 6H), 1.00 (d, J = 7.0 Hz, 6H), 0.97 (d, J = 7.0 Hz, 6H), | | |
| | 0.74-0.82 (m, 3H), 0.65-0.73 (m, 3H). | | |
| 110 | ¹H NMR (DMSO-d₆) (Rotamers present in NMR) d: 12.83 (s, 1H), | 1.05^{k} | 709.2 |
| | 9.60 (br. s, 0.3H), 9.13-9.21 (m, 0.3H), 9.02-9.06 (m, 0.7H), 9.01 (s, | | (M + H)⁺ |
| | 0.7H), 8.93 (d, J = 7.5 Hz, 1H), 8.73-8.80 (m, 0.3H), 8.53 (t, J = 5.8 Hz, | | |
| | 0.7H), 8.29 (s, 0.3H), 7.97-8.05 (m, 1H), 7.76 (s, 0.7H), | | |
| | 7.61-7.70 (m, 2H), 7.40-7.51 (m, 2H), 7.24-7.36 (m, 3H), 7.06-7.17 (m, | | |
| | 2H), 5.20 (dt, J = 7.8, 4.9 Hz, 0.7H), 4.94-5.04 (m, 0.3H), | | |
| | 4.47-4.75 (m, 2H), 4.32 (q, J = 7.2 Hz, 2H), 4.22 (dd, J = 14.8, 7.8 Hz, 0.3H), | | |
| | 3.52-3.61 (m, 0.7H), 2.93-3.16 (m, 2H), 2.53-2.63 (m, 1H), | | |
| | 1.48-1.59 (m, 2H), 1.45 (t, J = 6.9 Hz, 3H), 1.31-1.41 (m, 2H), | | |
| | 1.06-1.24 (m, 6H), 0.68-0.81 (m, 6H). | | |
| 111 | ¹H NMR (DMSO-d₆) (Rotamers present in NMR) d: 9.60 (s, 0.2H), | 1.19^{k} | 833.6 |
| | 9.29 (br. s, 0.8H), 9.18 (t, J = 5.5 Hz, 0.3H), 9.04 (t, J = 5.8 Hz, 0.8H), | | (M + H)⁺ |
| | 8.81-8.87 (m, 1H), 8.77 (t, J = 5.6 Hz, 0.2H), 8.53 (t, J = 5.6 Hz, 0.8H), | | |
| | 8.29 (s, 0.2H), 7.93 (d, J = 8.0 Hz, 0.8H), 7.85 (d, J = 7.5 Hz, 0.2H), | | |
| | 7.77 (s, 0.8H), 7.65 (s, 0.4H), 7.62 (s, 1.6H), 7.26-7.37 (m, 2H), | | |
| | 5.67-5.78 (m, 4H), 4.93-5.06 (m, 1H), 4.48-4.76 (m, 2H), | | |
| | 4.15-4.37 (m, 3H), 3.00-3.07 (m, 2H), 2.53-2.63 (m, 3H), 1.50-1.56 (m, J = 7.3 Hz, | | |
| | 1H), 1.47 (t, J = 6.9 Hz, 3H), 1.29-1.44 (m, 3H), 1.11-1.23 (m, | | |
| | 6H), 1.09 (d, J = 7.0 Hz, 6H), 1.04 (dd, J = 7.0, 1.0 Hz, 6H), | | |
| | 0.68-0.81 (m, 6H). | | |
| 112 | ¹H NMR (DMSO-d₆) (Rotamers present in NMR) d: 9.61 (s, 1H), | 1.28^{k} | 745.4 |
| | 9.18 (t, J = 5.8 Hz, 0.3H), 9.04 (t, J = 6.0 Hz, 0.7H), 8.81 (d, J = 7.8 Hz, | | (M + H)⁺ |
| | 1H), 8.75-8.79 (m, 0.3H), 8.53 (t, J = 5.9 Hz, 0.7H), 8.29 (s, 0.3H), | | |
| | 7.95-8.02 (m, 1H), 7.76 (s, 0.7H), 7.62-7.67 (m, 2H), 7.25-7.34 (m, | | |
| | 2H), 4.79 (dt, J = 7.9, 5.1 Hz, 1H), 4.48-4.75 (m, 2H), 4.28-4.39 (m, | | |
| | 2H), 3.57 (td, J = 9.5, 4.3 Hz, 1H), 2.73-2.89 (m, 2H), 2.54-2.64 (m, | | |
| | 1H), 1.46-1.57 (m, 5H), 1.43 (s, 9H), 1.40 (s, 9H), 1.06-1.22 (m, | | |
| | 8H), 0.66-0.81 (m, 6H). | | |
| 113 | ¹H NMR (DMSO-d₆) (Rotamers present in NMR) d: 12.64 (s, 1H), | 6.96^{a} | 661.3 |
| | 9.60 (s, 1H), 9.17 (t, J = 5.8 Hz, 0.3H), 9.03 (t, J = 6.0 Hz, 0.7H), | | (M + H)⁺ |
| | 8.83-8.90 (m, 1H), 8.77 (t, J = 6.1 Hz, 0.3H), 8.53 (t, J = 5.8 Hz, 0.7H), | | |
| | 8.29 (s, 0.3H), 7.98 (d, J = 8.0 Hz, 1H), 7.77 (s, 0.7H), 7.61-7.66 (m, 2H), | | |
| | 7.27-7.33 (m, 2H), 4.81-4.95 (m, 1H), 4.48-4.76 (m, 2H), | | |
| | 4.27-4.37 (m, 2H), 4.15 (q, J = 6.9 Hz, 2H), 3.53-3.62 (m, 1H), 2.77-2.97 (m, | | |
| | 2H), 2.54-2.65 (m, 1H), 1.44-1.57 (m, 5H), 1.32-1.44 (m, 2H), | | |
| | 1.08-1.22 (m, 9H), 0.67-0.82 (m, 6H). | | |
| 114 | ¹H NMR (DMSO-d₆) (Rotamers present in NMR) d: 12.59 (br. s, | 7.53^{a} | 689.4 |
| | 1H), 9.60 (s, 0.3H), 9.28 (s, 0.7H), 9.17 (t, J = 6.1 Hz, 0.3H), 9.03 (t, | | (M + H)⁺ |
| | J = 5.9 Hz, 0.7H), 8.83 (d, J = 8.0 Hz, 1H), 8.77 (t, J = 5.8 Hz, 0.3H), | | |
| | 8.52 (t, J = 5.9 Hz, 0.7H), 8.29 (s, 0.3H), 7.99 (d, J = 8.3 Hz, 1H), | | |
| | 7.77 (s, 0.7H), 7.60-7.67 (m, 2H), 7.26-7.34 (m, 2H), 4.80 (dt, J = 8.2, 5.0 Hz, | | |
| | 1H), 4.49-4.74 (m, 2H), 4.27-4.38 (m, 2H), 3.57 (td, J = 9.7, 4.4 Hz, | | |
| | 1H), 2.71-2.94 (m, 2H), 2.54-2.65 (m, 1H), 1.44-1.57 (m, 6H), | | |
| | 1.42 (s, 9H), 1.32-1.40 (m, 1H), 1.07-1.21 (m, 6H), 0.68-0.82 (m, | | |
| | 6H). | | |
| 115 | ¹H NMR (DMSO-d₆) (Rotamers present in NMR) d: 9.63 (s, 0.3H), | 7.52^{a} | 698.3 |
| | 9.29 (s, 0.7H), 9.24-9.30 (m, 0.3H), 9.14 (t, J = 6.0 Hz, 0.7H), 8.79 (t, | | (M + H)⁺ |
| | J = 5.8 Hz, 0.3H), 8.54 (t, J = 5.6 Hz, 0.7H), 8.29 (s, 0.3H), 7.80 (s, | | |
| | 1H), 7.76 (s, 0.7H), 7.69-7.76 (m, 1H), 7.25-7.31 (m, 2H), | | |
| | 7.11-7.18 (m, 1H), 5.60-5.74 (m, 4H), 4.45-4.73 (m, 2H), 4.03-4.26 (m, 2H), | | |
| | 3.57 (td, J = 9.5, 4.6 Hz, 1H), 2.54-2.65 (m, 1H), 2.01 (s, 6H), | | |
| | 1.44-1.58 (m, 2H), 1.31-1.43 (m, 5H), 1.06-1.21 (m, 6H), 0.65-0.89 (m, | | |
| | 6H). | | |
| 116 | ¹H NMR (DMSO-d₆) (Rotamers present in NMR) d: 12.69 (br. s, | 0.99^{k} | 767.5 |
| | 1H), 9.60 (s, 0.4H), 9.30 (br. s, 0.6H), 9.17 (t, J = 6.0 Hz, 0.4H), | | (M + H)⁺ |
| | 9.03 (t, J = 5.9 Hz, 0.6H), 8.89 (d, J = 8.0 Hz, 1H), 8.77 (t, J = 5.8 Hz, 0.4H), | | |
| | 8.52 (t, J = 5.8 Hz, 0.6H), 8.30 (s, 0.4H), 7.96-8.01 (m, 1.2H), | | |
| | 7.89-7.96 (m, 0.8H), 7.77 (s, 0.6H), 7.69-7.75 (m, 1H), 7.53-7.65 (m, 5H), | | |
| | 7.26-7.33 (m, 2H), 6.02 (s, 2H), 4.96-5.06 (m, 1H), 4.48-4.77 (m, | | |
| | 2H), 4.29 (q, J = 6.9 Hz, 2H), 3.57 (td, J = 9.5, 4.3 Hz, 1H), | | |
| | 2.79-3.00 (m, 2H), 2.53-2.65 (m, 1H), 1.46-1.58 (m, 2H), 1.43 (t, J = 6.9 Hz, | | |
| | 3H), 1.35-1.46 (m, 2H), 1.05-1.28 (m, 6H), 0.64-0.84 (m, 6H). | | |
| 117 | ¹H NMR (DMSO-d₆) (Rotamers present in NMR)d: 12.93 (br. s, 1H), | 7.21^{a} | 769.3 |
| | 12.56 (br. s, 1H), 8.87 (d, J = 7.8 Hz, 2H), 8.65-8.83 (m, 1H), | | (M + H)⁺ |
| | 8.15 (br. s, 1H), 8.01-8.09 (m, 1H), 7.98 (d, J = 8.3 Hz, 1H), 7.54-7.63 (m, | | |
| | 2H), 7.17-7.29 (m, 3H), 7.06-7.17 (m, 1H), 4.78-4.86 (m, 1H), | | |
| | 4.52-4.74 (m, 2H), 4.29 (q, J = 7.1 Hz, 2H), 3.93-4.08 (m, 1H), | | |
| | 2.75-2.97 (m, 2H), 2.59-2.73 (m, 1H), 2.44 (br. s, 3H), 1.62-1.75 (m, 1H), | | |
| | 1.47 (t, J = 6.9 Hz, 3H), 1.32-1.60 (m, 2H), 1.07-1.27 (m, 7H), | | |
| | 0.88-1.02 (m, 3H), 0.73 (t, J = 6.8 Hz, 3H). | | |
| 118 | ¹H NMR (DMSO-d₆) (Rotamers present in NMR)d: 12.98 (br. s, 1H), | 5.54^{a} | 744.3 |
| | 12.55 (br. s, 1H), 9.03 (t, J = 5.1 Hz, 1H), 8.88 (d, J = 7.8 Hz, 1H), | | (M + H)⁺ |
| | 8.58-8.77 (m, 1H), 8.48-8.57 (m, 1H), 8.26-8.38 (m, J = 5.8 Hz, 1H), | | |
| | 8.05 (br. s, 1H), 8.02 (d, J = 8.3 Hz, 1H), 7.59-7.66 (m, 2H), 7.30 (dd, | | |
| | J = 17.8, 3.5 Hz, 2H), 4.82 (dt, J = 7.8, 4.8 Hz, 1H), 4.47-4.69 (m, 2H), | | |
| | 4.31 (q, J = 7.2 Hz, 2H), 3.91 (t, J = 8.2 Hz, 1H), 3.17-3.30 (m, 2H), | | |
| | 2.75-3.02 (m, 4H), 2.53-2.61 (m, 1H), 1.95-2.13 (m, 2H), | | |
| | 1.73-1.88 (m, 2H), 1.56-1.71 (m, 1H), 1.48 (t, J = 6.9 Hz, 3H), 1.33-1.54 (m, | | |
| | 4H), 1.04-1.26 (m, 6H), 0.80-0.94 (m, 3H), 0.72 (t, J = 6.9 Hz, 3H). | | |
| 119 | ¹H NMR (DMSO-d₆) (Rotamers present in NMR) d: 12.95 (br. s, | 1.10^{k} | 805.6 |
| | 1H), 12.52 (br. s, 1H), 8.68-8.91 (m, 2.2H), 8.15-8.23 (m, 2.7H), | | (M + H)⁺ |
| | 7.98 (d, J = 8.0 Hz, 1H), 7.75-7.90 (m, 1.8H), 7.50-7.58 (m, 1.8H), | | |
| | 7.19 (br. s, 1.7H), 4.78-4.86 (m, 1H), 4.54-4.75 (m, 2H), 4.28 (q, | | |
| | J = 7.0 Hz, 2H), 3.96-4.09 (m, 1H), 2.76-2.95 (m, 2H), 2.65-2.76 (m, | | |
| | 1H), 1.64-1.79 (m, 1H), 1.41-1.58 (m, 5.2H), 1.10-1.28 (m, 6H), | | |
| | 0.96 (t, J = 7.0 Hz, 2.6H), 0.69-0.79 (m, 2.8H). | | |
| 120 | ¹H NMR (DMSO-d₆) (Rotamers present in NMR)d: 12.96 (br. s, 1H), | 1.03^{k} | 782.6 |
| | 12.54 (br. s, 1H), 8.88 (d, J = 7.8 Hz, 2H), 8.59-9.02 (m, 2H), | | (M + H)⁺ |
| | 8.15-8.31 (m, 4H), 7.97 (d, J = 8.3 Hz, 1H), 7.47-7.59 (m, 2H), | | |
| | 7.15-7.26 (m, 2H), 4.82 (dt, J = 7.9, 4.8 Hz, 1H), 4.53-4.74 (m, 2H), 4.28 (q, | | |
| | J = 7.3 Hz, 2H), 3.97-4.11 (m, 1H), 2.76-2.96 (m, 2H), 2.66-2.76 (m, | | |
| | 1H), 1.65-1.80 (m, J = 9.3 Hz, 1H), 1.47 (t, J = 6.9 Hz, 3H), | | |
| | 1.34-1.60 (m, 2H), 1.07-1.30 (m, 7H), 0.96 (t, J = 7.0 Hz, 3H), 0.75 (t, J = 6.4 Hz, | | |
| | 3H). | | |
| 121 | ¹H NMR (DMSO-d₆) (Rotamers present in NMR) d: 9.87-11.08 (m, | 0.90^{k} | 753.6 |
| | 0.5H), 9.00 (d, J = 5.5 Hz, 1H), 8.90 (br. s, 1.4H), 8.26 (s, 0.1H), | | (M + H)⁺ |
| | 8.09 (s, 0.9H), 8.01 (d, J = 8.3 Hz, 1H), 7.85-7.94 (m, 2H), 7.54-7.63 (m, | | |
| | 2H), 7.20-7.29 (m, 2H), 7.09 (br. s, 1H), 6.81-6.90 (m, 2H), | | |
| | 4.53-4.75 (m, 2H), 4.40 (dd, J = 18.6, 7.3 Hz, 1H), 4.23-4.35 (m, 2H), | | |
| | 4.11 (br. s, 0.6H), 3.95 (br. s, 0.8H), 2.53-2.75 (m, 3H), 1.35-1.79 (m, | | |
| | 7H), 1.09-1.26 (m, 6H), 0.96 (t, J = 7.2 Hz, 3H), 0.72 (t, J = 6.9 Hz, | | |
| | 3H). | | |
| 122 | ¹H NMR (DMSO-d₆) (Rotamers present in NMR)d: 12.92 (br. s, 1H), | 1.00^{k} | 785.6 |
| | 12.49 (br. s, 1H), 8.79-9.13 (m, 1H), 8.87 (d, J = 7.8 Hz, 1H), | | (M + H)⁺ |
| | 8.53-8.78 (m, 1H), 8.07-8.36 (m, 1H), 7.98 (d, J = 8.0 Hz, 1H), | | |
| | 7.85-8.02 (m, 1H), 7.58 (d, J = 8.0 Hz, 1H), 7.55 (s, 1H), 7.17-7.29 (m, 2H), | | |
| | 6.89-7.00 (m, 1H), 6.73-6.88 (m, 1H), 4.75-4.86 (m, 1H), 4.68 (quin, | | |
| | J = 6.4 Hz, 1H), 4.56 (quin, J = 6.4 Hz, 1H), 4.28 (q, J = 7.0 Hz, 2H), | | |
| | 3.90-4.07 (m, 1H), 3.83 (s, 3H), 2.91 (dd, J = 16.6, 5.4 Hz, 1H), | | |
| | 2.79 (dd, J = 16.6, 5.4 Hz, 1H), 2.56-2.71 (m, 1H), 1.62-1.78 (m, 1H), | | |
| | 1.46 (t, J = 7.0 Hz, 3H), 1.32-1.58 (m, 3H), 1.04-1.28 (m, 6H), | | |
| | 0.83-1.01 (m, 3H), 0.72 (t, J = 6.8 Hz, 3H). | | |
| 123 | ¹H NMR (DMSO-d₆) (Rotamers present in NMR) d: 12.95 (br. s, | 1.09^{k} | 835.6 |
| | 1H), 12.54 (br. s, 1H), 8.89-9.15 (m, 1H), 8.86 (d, J = 7.6 Hz, 1H), | | (M + H)⁺ |
| | 8.56-8.79 (m, 1H), 8.10-8.29 (m, 1H), 8.07 (d, J = 8.4 Hz, 1H), | | |
| | 7.98 (d, J = 8.2 Hz, 1H), 7.60 (d, J = 8.2 Hz, 1H), 7.58 (s, 1H), | | |
| | 7.31-7.43 (m, 1H), 7.27 (dd, J = 6.8, 3.6 Hz, 2H), 7.18-7.31 (m, 1H), | | |
| | 4.76-4.85 (m, 1H), 4.66 (quin, J = 6.4 Hz, 1H), 4.57 (quin, J = 6.4 Hz, 1H), | | |
| | 4.28 (q, J = 7.0 Hz, 2H), 3.93-4.04 (m, 1H), 3.88 (s, 3H), 2.90 (dd, J = 16.6, | | |
| | 5.4 Hz, 1H), 2.79 (dd, J = 16.6, 5.2 Hz, 1H), 2.58-2.68 (m, 1H), | | |
| | 1.63-1.76 (m, 1H), 1.46 (t, J = 7.2 Hz, 3H), 1.31-1.58 (m, 3H), | | |
| | 1.04-1.26 (m, 6H), 0.84-1.02 (m, 3H), 0.72 (t, J = 7.0 Hz, 3H). | | |
| 124 | ¹H NMR (DMSO-d₆) (Rotamers present in NMR) d: 12.94 (br. s, | 1.10^{k} | 851.6 |
| | 1H), 12.54 (br. s, 1H), 8.90-9.16 (m, 1H), 8.86 (d, J = 7.2 Hz, 1H), | | (M + H)⁺ |
| | 8.56-8.82 (m, 1H), 8.04-8.30 (m, 2H), 7.98 (d, J = 8.0 Hz, 1H), | | |
| | 7.59 (d, J = 8.0 Hz, 1H), 7.57 (s, 1H), 7.21-7.30 (m, 2H), 6.99-7.09 (m, | | |
| | 2H), 4.77-4.85 (m, 1H), 4.66 (quin, J = 6.4 Hz, 1H), 4.56 (quin, J = 6.4 Hz, | | |
| | 1H), 4.28 (q, J = 7.0 Hz, 2H), 3.93-4.05 (m, 1H), 3.86 (s, 3H), | | |
| | 2.90 (dd, J = 17.0, 5.0 Hz, 1H), 2.79 (dd, J = 17.0, 5.0 Hz, 1H), | | |
| | 2.58-2.67 (m, 1H), 1.61-1.76 (m, 1H), 1.46 (t, J = 7.2 Hz, 3H), | | |
| | 1.35-1.56 (m, 2H), 1.04-1.25 (m, 7H), 0.84-0.99 (m, 3H), 0.71 (t, J = 6.8 Hz, | | |
| | 3H). | | |
| 125 | ¹H NMR (DMSO-d₆) (Rotamers present in NMR) d: 11.34 (br. s, | 0.92^{k} | 715.5 |
| | 2H), 8.75-8.99 (m, 1H), 8.32-8.42 (m, 1H), 8.14 (s, 1H), | | (M + H)⁺ |
| | 7.92-8.07 (m, 3H), 7.46-7.72 (m, 6H), 7.22 (s, 2H), 4.51-4.78 (m, 2H), 4.29 (q, | | |
| | J = 6.9 Hz, 2H), 3.91-4.08 (m, 1H), 3.57 (dd, J = 13.2, 5.4 Hz, 2H), | | |
| | 2.63-2.78 (m, 1H), 1.62-1.77 (m, J = 5.5 Hz, 1H), 1.48 (t, J = 7.0 Hz, | | |
| | 3H), 1.30-1.58 (m, 2H), 1.06-1.27 (m, 7H), 0.96 (t, J = 7.2 Hz, 3H), | | |
| | 0.73 (t, J = 6.9 Hz, 3H). | | |
| 126 | ¹H NMR (DMSO-d₆): d = 12.95 (br. s, 1H), 12.54 (br. s, 1H), | 1.14^{k} | 819.6 |
| | 8.80-9.16 (m, 1H), 8.86 (d, J = 7.6 Hz, 1H), 8.41-8.79 (m, 1H), | | (M + H)⁺ |
| | 8.14-8.25 (m, 1H), 8.08 (d, J = 8.0 Hz, 1H), 7.97 (d, J = 8.0 Hz, 1H), | | |
| | 7.68-7.79 (m, 1H), 7.50-7.67 (m, 3H), 6.48-7.30 (m, 2H), 4.76-4.87 (m, 1H), | | |
| | 4.68 (quin, J = 6.4 Hz, 1H), 4.57 (quin, J = 6.4 Hz, 1H), 4.28 (q, J = 7.0 Hz, | | |
| | 2H), 3.95-4.11 (m, 1H), 2.90 (dd, J = 16.8, 5.0 Hz, 1H), 2.79 (dd, | | |
| | J = 16.8, 5.0 Hz, 1H), 2.58-2.71 (m, 1H), 2.50 (s, 3H, under DMSO | | |
| | peak), 1.62-1.78 (m, 1H), 1.33-1.59 (m, 3H), 1.46 (t, J = 7.0 Hz, 3H), | | |
| | 1.03-1.29 (m, 6H), 0.83-1.02 (m, 3H), 0.72 (t, J = 6.8 Hz, 3H). | | |
| 127 | ¹H NMR (DMSO-d₆) (Rotamers present in NMR) d: 12.96 (br. s, | 6.82^{b} | 804.4 |
| | 1H), 12.56 (br. s, 1H), 9.28 (s, 2H), 8.91-9.04 (m, 1H), 8.88 (d, | | (M + H)⁺ |
| | J = 7.8 Hz, 1H), 8.64-8.81 (m, 1H), 8.09-8.23 (m, 3H), 7.97 (d, J = 8.3 Hz, | | |
| | 1H), 7.81 (d, J = 8.3 Hz, 2H), 7.51-7.58 (m, 2H), 7.15-7.24 (m, | | |
| | 2H), 4.79-4.85 (m, 1H), 4.53-4.78 (m, 2H), 4.22-4.34 (m, 2H), | | |
| | 3.96-4.09 (m, 1H), 2.76-2.96 (m, 2H), 2.67-2.75 (m, 1H), 1.66-1.79 (m, | | |
| | 1H), 1.47 (t, J = 6.9 Hz, 3H), 1.31-1.58 (m, 2H), 1.05-1.30 (m, 7H), | | |
| | 0.97 (t, J = 7.2 Hz, 3H), 0.74 (t, J = 6.9 Hz, 3H). | | |
| 128 | ¹H NMR (DMSO-d₆) (Rotamers present in NMR) d: 12.94 (br. s, | 8.08^{b} | 802.3 |
| | 1H), 12.54 (br. s, 1H), 8.87 (d, J = 7.8 Hz, 1H), 8.71-8.95 (m, 2H), | | (M + H)⁺ |
| | 8.18 (s, 1H), 7.96 (d, J = 8.0 Hz, 2H), 7.86-7.94 (m, 2H), | | |
| | 7.50-7.61 (m, 3H), 7.40 (t, J = 2.1 Hz, 2H), 7.17-7.26 (m, 2H), 6.30 (t, J = 2.1 Hz, | | |
| | 2H), 4.82 (dt, J = 7.8, 4.9 Hz, 1H), 4.54-4.78 (m, 2H), 4.25 (quin, | | |
| | J = 6.8 Hz, 2H), 4.03 (t, J = 9.8 Hz, 1H), 2.75-2.99 (m, 2H), | | |
| | 2.65-2.79 (m, 1H), 1.65-1.80 (m, 1H), 1.46 (t, J = 6.9 Hz, 3H), 1.33-1.60 (m, | | |
| | 2H), 1.05-1.31 (m, 7H), 0.98 (t, J = 7.2 Hz, 3H), 0.74 (t, J = 6.8 Hz, | | |
| | 3H). | | |
| 129 | ¹H NMR (DMSO-d₆) (Rotamers present in NMR) d: 12.94 (br. s, | 7.37^{b} | 844.3 |
| | 1H), 12.49 (s, 1H), 8.88 (d, J = 7.8 Hz, 1H), 8.73-8.95 (m, 2H), | | (M + H)⁺ |
| | 8.15 (s, 1H), 7.96-8.07 (m, 3H), 7.53-7.61 (m, 2H), 7.48 (d, J = 8.3 Hz, | | |
| | 2H), 7.22 (s, 2H), 4.82 (dt, J = 7.8, 4.9 Hz, 1H), 4.52-4.77 (m, 2H), | | |
| | 4.29 (q, J = 7.0 Hz, 2H), 4.00 (t, J = 9.7 Hz, 1H), 3.28 (s, 3H), 3.01 (s, | | |
| | 3H), 2.75-2.97 (m, 2H), 2.65-2.74 (m, 1H), 1.62-1.78 (m, 1H), | | |
| | 1.47 (t, J = 6.9 Hz, 3H), 1.37-1.58 (m, 2H), 1.09-1.28 (m, 7H), 0.96 (t, | | |
| | J = 7.2 Hz, 3H), 0.74 (t, J = 6.8 Hz, 3H). | | |
| 130 | ¹H NMR (DMSO-d₆) (Rotamers present in NMR) d: 12.95 (br. s, | 7.54^{b} | 803.3 |
| | 1H), 12.54 (br. s, 1H), 8.87 (d, J = 7.5 Hz, 1H), 8.74-9.02 (m, 2H), | | (M + H)⁺ |
| | 8.63 (d, J = 2.8 Hz, 1H), 8.16 (s, 1H), 8.11 (d, J = 8.8 Hz, 2H), | | |
| | 7.92-7.99 (m, 3H), 7.85 (d, J = 1.8 Hz, 1H), 7.49-7.58 (m, 2H), | | |
| | 7.14-7.24 (m, 2H), 6.61-6.64 (m, 1H), 4.78-4.86 (m, 1H), 4.55-4.77 (m, 2H), | | |
| | 4.21-4.32 (m, 2H), 3.95-4.06 (m, 1H), 2.76-2.97 (m, 2H), | | |
| | 2.69-2.79 (m, 1H), 1.63-1.79 (m, 1H), 1.46 (t, J = 6.9 Hz, 3H), 1.35-1.58 (m, | | |
| | 2H), 1.08-1.27 (m, 7H), 0.98 (t, J = 7.3 Hz, 3H), 0.74 (t, J = 6.8 Hz, | | |
| | 3H). | | |
| 131 | ¹H NMR (DMSO-d₆) (Rotamers present in NMR) d:12.22-13.13 (br. | 6.33^{b} | 803.3 |
| | s, 2H), 9.06 (br. s, 1H), 8.87 (d, J = 7.5 Hz, 1H), 8.75-8.97 (m, 2H), | | (M + H)⁺ |
| | 8.17 (d, J = 8.3 Hz, 3H), 8.11 (s, 1H), 7.97 (d, J = 8.0 Hz, 1H), 7.83 (d, | | |
| | J = 6.5 Hz, 2H), 7.49-7.59 (m, 3H), 7.16-7.23 (m, 2H), 4.79-4.86 (m, | | |
| | 1H), 4.55-4.77 (m, 2H), 4.23-4.33 (m, 2H), 4.03 (t, J = 9.0 Hz, 1H), | | |
| | 2.76-2.96 (m, 2H), 2.69-2.80 (m, 1H), 1.64-1.80 (m, 1H), 1.47 (t, | | |
| | J = 6.9 Hz, 3H), 1.36-1.58 (m, 2H), 1.09-1.29 (m, 7H), 0.97 (t, J = 7.2 Hz, | | |
| | 3H), 0.74 (t, J = 6.9 Hz, 3H). | | |
| 132 | ¹H NMR (DMSO-d₆) (Rotamers present in NMR) d: 8.60-9.16 (m, | 0.93^{k} | 745.6 |
| | 2H), 8.25-8.43 (m, 1H), 8.04-8.16 (m, 0.8H), 7.99 (d, J = 8.3 Hz, 1H), | | (M + H)⁺ |
| | 7.94 (d, J = 8.8 Hz, 2H), 7.56 (d, J = 8.4 Hz, 1H), 7.54 (s, 1H), | | |
| | 7.14-7.26 (m, 2H), 6.87-7.04 (m, 2H), 6.50-6.57 (m, 0.2H), 4.70 (quin, | | |
| | J = 6.4 Hz, 1H), 4.59 (quin, J = 6.4 Hz, 1H), 4.28 (q, J = 7.0 Hz, 2H), | | |
| | 3.91-4.01 (m, 1H), 3.80 (s, 3H), 3.59 (dd, J = 13.2, 5.4 Hz, 2H), | | |
| | 2.63-2.75 (m, 1H), 1.61-1.75 (m, 1H), 1.47 (t, J = 6.9 Hz, 3H), | | |
| | 1.38-1.56 (m, 2H), 1.07-1.27 (m, 7H), 0.95 (t, J = 7.2 Hz, 2.4H), 0.90 (t, J = 7.0 Hz, | | |
| | 0.6H), 0.73 (t, J = 6.9 Hz, 3H). | | |
| 133 | ¹H NMR (DMSO-d₆) (Rotamers present in NMR) d: 8.55-9.13 (m, | 0.97^{k} | 759.7 |
| | 2H), 8.24-8.42 (m, 1H), 8.06-8.16 (m, 1H), 7.99 (d, J = 8.0 Hz, 1H), | | (M + H)⁺ |
| | 7.88-8.04 (m, 1H), 7.57 (d, J = 8.4 Hz, 1H), 7.55 (s, 1H), | | |
| | 7.14-7.27 (m, 2H), 6.82-6.90 (m, 1H), 6.71-6.81 (m, 0.7H), 6.50-6.60 (m, | | |
| | 0.3H), 4.69 (quin, J = 6.4 Hz, 1H), 4.58 (quin, J = 6.4 Hz, 1H), 4.28 (q, | | |
| | J = 7.0 Hz, 2H), 3.89-4.05 (m, 1H), 3.79 (s, 3H), 3.51-3.64 (m, 2H), | | |
| | 2.62-2.70 (m, 1H), 2.40 (s, 3H), 1.61-1.75 (m, 1H), 1.47 (t, J = 6.9 Hz, | | |
| | 3H), 1.33-1.55 (m, 2H), 1.06-1.28 (m, 7H), 0.83-1.00 (m, 3H), | | |
| | 0.72 (t, J = 6.8 Hz, 3H). | | |
| 134 | ¹H NMR (DMSO-d₆) (Rotamers present in NMR) d: 8.80-9.15 (m, | 0.75^{k} | 828.7 |
| | 1H), 8.50-8.79 (m, 1H), 8.30-8.41 (m, 1H), 8.09-8.21 (m, 1H), | | (M + H)⁺ |
| | 7.99 (d, J = 8.0 Hz, 1H), 7.90-8.07 (m, 1H), 7.59 (d, J = 8.4 Hz, 1H), | | |
| | 7.57 (s, 1H), 7.30-7.46 (m, 1.7H), 7.24 (dd, J = 12.4, 3.0 Hz, 2H), | | |
| | 6.48-6.60 (m, 0.3H), 4.68 (quin, J = 6.4 Hz, 1H), 4.57 (quin, J = 6.4 Hz, 1H), | | |
| | 4.29 (q, J = 7.0 Hz, 2H), 3.97-4.10 (m, 1H), 3.47-3.86 (m, 4H), | | |
| | 3.58 (dd, J = 13.1, 5.5 Hz, 2H), 2.92-3.24 (m, 4H), 2.62-2.71 (m, 1H), | | |
| | 2.45 (s, 3H), 1.63-1.79 (m, 1H), 1.47 (t, J = 6.9 Hz, 3H), | | |
| | 1.33-1.60 (m, 3H), 1.04-1.28 (m, 6H), 0.84-1.03 (m, 3H), 0.72 (t, J = 6.9 Hz, | | |
| | 3H). | | |
| 135 | ¹H NMR (DMSO-d₆) (Rotamers present in NMR) d: 12.95 (br. s., | 2.55^{b} | 841.4 |
| | 1H), 12.54 (br. s., 1H), 8.88 (d, J = 7.8 Hz, 1H), 8.82 (br. s., 2H), | | (M + H)⁺ |
| | 8.11 (s, 1H), 7.91-8.03 (m, 3H), 7.51-7.62 (m, 2H), 7.23 (s, 2H), | | |
| | 6.93-7.04 (m, 2H), 4.78-4.86 (m, 1H), 4.52-4.77 (m, 2H), 4.29 (q, J = 6.9 Hz, | | |
| | 2H), 4.10-4.19 (m, 2H), 3.91-4.03 (m, 1H), 3.72-3.79 (m, 2H), | | |
| | 3.46-3.56 (m, 4H), 2.75-2.97 (m, 2H), 2.64-2.76 (m, 1H), | | |
| | 1.62-1.76 (m, 1H), 1.47 (t, J = 7.0 Hz, 3H), 1.36-1.57 (m, 2H), 1.06-1.28 (m, | | |
| | 7H), 0.96 (t, J = 7.2 Hz, 3H), 0.73 (t, J = 6.9 Hz, 3H). | | |
| 136 | ¹H NMR (DMSO-d₆) (Rotamers present in NMR) d: 8.63-8.85 (m, | 3.01^{a} | 514.3 |
| | 2H), 8.08 (s, 0.1H), 8.00 (s, 0.9H), 7.88-7.95 (m, 2H), 7.42-7.49 (m, | | (M + H)⁺ |
| | 2H), 7.33-7.41 (m, 1H), 7.23 (d, J = 3.5 Hz, 1H), 7.10 (d, J = 3.5 Hz, | | |
| | 1H), 4.47-4.70 (m, 2H), 3.86 (m, 1H), 2.55 (m, 1H), 1.64 (m, 1H), | | |
| | 1.43 (m, 2H), 1.04-1.26 (m, 16H), 0.81-0.94 (m, 3H), 0.72 (t, J = 6.9 Hz, | | |
| | 3H). | | |
| 137 | ¹H NMR (DMSO-d₆) (Rotamers present in NMR) d: 9.04-9.11 (m, | 2.99^{b} | 548.3 |
| | 0.1H), 8.86-8.94 (m, 0.9H), 8.54-8.64 (m, 0.9H), 8.44-8.52 (m, | | (M + H)⁺ |
| | 0.1H), 8.16 (s, 0.1H), 8.05 (s, 0.9H), 7.91 (d, J = 7.8 Hz, 2H), | | |
| | 7.40-7.51 (m, 2H), 7.34-7.40 (m, 1H), 7.29-7.33 (m, 4H), 7.25-7.29 (m, | | |
| | 1H), 7.24 (d, J = 3.5 Hz, 1H), 7.09 (d, J = 3.5 Hz, 1H), 4.61-4.70 (m, | | |
| | 1H), 4.47-4.56 (m, 1H), 4.26-4.35 (m, 1H), 3.75-3.93 (m, 3H), | | |
| | 2.53-2.64 (m, 1H), 1.54-1.67 (m, J = 6.3 Hz, 1H), 1.43-1.53 (m, 1H), | | |
| | 1.31-1.43 (m, 1H), 1.07-1.26 (m, 6H), 0.76-0.88 (m, 3H), 0.72 (t, J = 6.8 Hz, | | |
| | 3H). | | |
| 138 | ¹H NMR (DMSO-d₆) (Rotamers present in NMR) d: 8.97-9.04 (m, | 7.51^{a} | 488.3 |
| | 0.1H), 8.86-8.93 (m, 0.9H), 8.54-8.63 (m, 0.9H), 8.46-8.51 (m, | | (M + H)⁺ |
| | 0.1H), 8.41-8.45 (m, 0.1H), 8.09 (s, 0.9H), 7.92 (d, J = 0.5 Hz, 2H), | | |
| | 7.42-7.51 (m, 2H), 7.34-7.42 (m, 1H), 7.23 (d, J = 3.8 Hz, 1H), | | |
| | 7.09 (d, J = 3.5 Hz, 1H), 4.61-4.70 (m, 1H), 4.44-4.61 (m, 1H), | | |
| | 4.28-4.38 (m, 1H), 3.91-3.99 (m, 1H), 3.79 (s, 3H), 2.54-2.64 (m, 1H), | | |
| | 1.58-1.72 (m, 1H), 1.34-1.52 (m, 3H), 1.05-1.25 (m, 6H), 0.80-0.93 (m, | | |
| | 3H), 0.72 (t, J = 6.7 Hz, 3H). | | |
| 139 | ¹H NMR (DMSO-d₆) (Rotamers present in NMR) d: 8.86 (t, J = 5.9 Hz, | 2.98^{b} | 530.3 |
| | 1H), 8.55-8.63 (m, 1H), 8.03 (s, 1H), 7.91 (d, J = 7.8 Hz, 2H), | | (M + H)⁺ |
| | 7.42-7.51 (m, 2H), 7.33-7.41 (m, 1H), 7.23 (d, J = 3.5 Hz, 1H), | | |
| | 7.09 (d, J = 3.5 Hz, 1H), 4.60-4.72 (m, 1H), 4.46-4.56 (m, 1H), | | |
| | 3.83-3.97 (m, 1H), 2.55-2.64 (m, 1H), 1.59-1.72 (m, J = 6.5 Hz, 1H), | | |
| | 1.29-1.51 (m, 12H), 1.05-1.26 (m, 6H), 0.78-0.94 (m, 3H), 0.67-0.76 (m, 3H). | | |
| 140 | ¹H NMR (DMSO-d₆) (Rotamers present in NMR) d: 9.06-9.15 (m, | 2.99^{b} | 534.3 |
| | 0.1H), 8.88-8.98 (m, 0.9H), 8.76-8.86 (m, 0.9H), 8.61-8.71 (m, | | (M + H)⁺ |
| | 0.1H), 8.36 (br. s, 0.1H), 8.13 (br. s, 0.9H), 8.00-8.08 (m, 2H), | | |
| | 7.88 (d, J = 8.0 Hz, 2H), 7.70 (t, J = 7.3 Hz, 1H), 7.50-7.59 (m, 2H), | | |
| | 7.41-7.49 (m, 2H), 7.33-7.40 (m, 1H), 7.19-7.23 (m, 1H), 7.02-7.09 (m, | | |
| | 1H), 4.62-4.71 (m, 1H), 4.48-4.61 (m, 1H), 3.96-4.07 (m, 1H), | | |
| | 2.66-2.75 (m, 1H), 1.64-1.75 (m, 1H), 1.39-1.55 (m, 3H), 1.06-1.26 (m, | | |
| | 6H), 0.90-1.01 (m, 3H), 0.71 (t, J = 6.1 Hz, 3H). | | |
| 141 | ¹H NMR (MEOH-d₄) (Rotamers present in NMR) d: 8.88 (t, J = 5.9 Hz, | 6.48^{a} | 666.3 |
| | 1H), 8.42 (s, 1H), 8.01 (s, 1H), 7.81 (d, J = 13.8 Hz, 1H), 7.66 (s, | | (M + H)⁺ |
| | 1H), 7.29-7.35 (m, 1H), 7.27 (d, J = 3.8 Hz, 1H), 7.03 (d, J = 3.5 Hz, | | |
| | 1H), 4.68-4.88 (m, 3H), 4.19 (q, J = 7.0 Hz, 2H), 3.67-3.78 (m, 1H), | | |
| | 2.61-2.72 (m, J = 9.6, 9.6, 5.6 Hz, 1H), 2.05 (s, 3H), 1.73-1.86 (m, | | |
| | 1H), 1.62-1.72 (m, 1H), 1.52-1.61 (m, 2H), 1.46 (t, J = 7.0 Hz, 3H), | | |
| | 1.17-1.37 (m, 7H), 0.94 (t, J = 7.3 Hz, 3H), 0.82 (t, J = 6.5 Hz, 3H). | | |
| 142 | ¹H NMR (DMSO-d₆) (Rotamers present in NMR) d: 9.53-9.70 (m, | 0.94^{e} | 705.6 |
| | 0.3H), 9.22-9.41 (m, 0.7H), 9.18 (t, J = 6.0 Hz, 0.3H), 9.01 (t, J = 6.1 Hz, | | (M + H)⁺ |
| | 0.7H), 8.97 (d, J = 7.5 Hz, 1H), 8.79 (t, J = 1.0 Hz, 0.3H), 8.55 (t, | | |
| | J = 4.8 Hz, 0.7H), 8.26-8.31 (s, 0.3H), 7.98 (dd, J = 8.0 Hz, 1H), | | |
| | 7.76 (s, 0.7H), 7.67-7.74 (m, 1H), 7.58-7.66 (m, J = 1.3 Hz, 1H), | | |
| | 7.31-7.35 (m, J = 3.8 Hz, 1H), 7.27-7.30 (m, 1H), 5.11 (s, 2H), 4.89-4.96 (m, | | |
| | 1H), 4.64-4.75 (m, J = 6.5 Hz, 1H), 4.50-4.62 (m, 0.7H), | | |
| | 4.17-4.29 (m, 0.3H), 3.77 (s, 3H), 3.67 (s, 3H), 3.63 (s, 3H), 3.57 (td, J = 9.5, | | |
| | 4.8 Hz, 1H), 2.95 (ddd, J = 11.5 Hz, 2H), 2.54-2.64 (m, 1H), | | |
| | 1.45-1.62 (m, 2H), 1.40 (d, J = 6.3 Hz, 2H), 1.05-1.22 (m, 6H), | | |
| | 0.66-0.83 (m, 6H). | | |
| 143 | ¹H NMR (DMSO-d₆) (Rotamers present in NMR) d: 9.60 (s, 0.3H), | 1.07^{k} | 689.5 |
| | 9.28 (br. s, 0.7H), 9.17 (t, J = 6.1 Hz, 0.3H), 9.03 (t, J = 6.0 Hz, 0.7H), | | (M + H)⁺ |
| | 8.84 (d, J = 7.5 Hz, 1H), 8.77 (t, J = 5.9 Hz, 0.3H), 8.52 (t, J = 5.8 Hz, | | |
| | 0.7H), 8.29 (s, 0.3H), 7.96 (d, J = 8.3 Hz, 1H), 7.77 (s, 0.7H), | | |
| | 7.60-7.66 (m, 2H), 7.27-7.33 (m, 2H), 4.94 (dt, J = 7.7, 5.3 Hz, 1H), | | |
| | 4.48-4.74 (m, 2H), 4.28-4.37 (m, 2H), 4.16 (qd, J = 7.1, 2.9 Hz, 2H), | | |
| | 4.09 (q, J = 7.1 Hz, 2H), 3.58 (dt, J = 9.6, 4.9 Hz, 1H), 2.86-3.03 (m, 2H), | | |
| | 2.53-2.65 (m, 1H), 1.48 (t, J = 6.9 Hz, 3H), 1.44-1.57 (m, 1H), | | |
| | 1.31-1.43 (m, 2H), 1.08-1.26 (m, 13H), 0.75-0.82 (m, 3H), 0.68-0.75 (m, | | |
| | 3H). | | |
| 200 | ¹H NMR (DMSO-d₆) (Rotamers present in NMR) d: 9.14 (t, J = 6.3 Hz, | 0.89^{e} | 540.5 |
| | 1H), 8.02 (br. s, 2H), 7.90-7.95 (m, 2H), 7.44-7.51 (m, 2H), | | (M + H)⁺ |
| | 7.35-7.41 (m, 1H), 7.28 (d, J = 3.5 Hz, 1H), 7.11 (d, J = 3.8 Hz, 1H), | | |
| | 4.85-5.04 (m, 1H), 4.59-4.76 (m, 1H), 4.44-4.53 (m, 2H), | | |
| | 2.62-2.78 (m, 1H), 2.10-2.31 (m, 1H), 1.64-1.86 (m, 1H), 1.35-1.54 (m, 3H), | | |
| | 1.26-1.35 (m, 1H), 1.05-1.25 (m, 5H), 0.94 (t, J = 7.3 Hz, 3H), 0.78 (t, | | |
| | J = 6.7 Hz, 3H). | | |
| 201 | ¹H NMR (DMSO-d₆) (Rotamers present in NMR) d: 11.83-13.17 (m, | 0.73^{k} | 847.1 |
| | 2H), 8.92-9.10 (m, 1H), 8.88 (d, J = 7.8 Hz, 1H), 8.07-8.21 (m, 1H), | | (M + H)⁺ |
| | 7.99 (d, J = 8.3 Hz, 1H), 7.53-7.67 (m, 2H), 7.32-7.40 (m, 2H), | | |
| | 7.25 (dd, J = 18.7, 3.4 Hz, 3H), 7.00 (d, J = 7.3 Hz, 1H), 4.78-4.87 (m, 1H), | | |
| | 4.44-4.78 (m, 2H), 4.21-4.38 (m, 2H), 3.90-4.15 (m, 1H), | | |
| | 2.73-2.97 (m, 2H), 2.54-2.66 (m, 1H), 2.33 (s., 3H), 1.52-1.72 (m, 2H), | | |
| | 1.35-1.51 (m, 4H), 1.06-1.28 (m, 7H), 0.79-0.99 (m, 3H), 0.73 (t, J = 6.4 Hz, | | |
| | 3H). | | |
| 202 | ¹H NMR (DMSO-d₆) (Rotamers present in NMR) d: 13.10 (br. s, | 1.51^{b} | 578.2 |
| | 1H), 8.96-9.04 (m, 1H), 8.72-8.94 (m, 1H), 8.14 (br. s, 1H), | | (M + H)⁺ |
| | 8.01-8.07 (m, 3H), 7.95-8.01 (m, 3H), 7.67-7.74 (m, 1H), 7.53 (t, J = 7.7 Hz, | | |
| | 2H), 7.18-7.27 (m, 2H), 4.50-4.74 (m, 2H), 4.03 (q, J = 7.0 Hz, | | |
| | 1H), 2.64-2.78 (m, 1H), 1.70 (d, J = 5.0 Hz, 1H), 1.29-1.56 (m, 3H), | | |
| | 1.06-1.27 (m, 6H), 0.88-1.00 (m, 3H), 0.71 (t, J = 6.9 Hz, 3H). | | |
| 203 | ¹H NMR (DMSO-d₆) (Rotamers present in NMR) d: 9.87-11.08 (m, | 0.90^{k} | 753.6 |
| | 0.5H), 9.00 (d, J = 5.5 Hz, 1H), 8.90 (br. s, 1.4H), 8.26 (s, 0.1H), | | (M + H)⁺ |
| | 8.09 (s, 0.9H), 8.01 (d, J = 8.3 Hz, 1H), 7.85-7.94 (m, 2H), 7.54-7.63 (m, | | |
| | 2H), 7.20-7.29 (m, 2H), 7.09 (br. s, 1H), 6.81-6.90 (m, 2H), | | |
| | 4.53-4.75 (m, 2H), 4.40 (dd, J = 18.6, 7.3 Hz, 1H), 4.23-4.35 (m, 2H), | | |
| | 4.11 (br. s, 0.6H), 3.95 (br. s, 0.8H), 2.53-2.75 (m, 3H), 1.35-1.79 (m, | | |
| | 7H), 1.09-1.26 (m, 6H), 0.96 (t, J = 7.2 Hz, 3H), 0.72 (t, J = 6.9 Hz, | | |
| | 3H). | | |
| 204 | ¹H NMR (DMSO-d₆) (Rotamers present in NMR) d: 12.73 (s, 1H), | 0.90^{k} | 745.6 |
| | 9.60 (s, 0.7H), 9.51 (s, 0.3H), 9.18 (t, J = 5.8 Hz, 0.3H), 9.04 (t, J = 6.0 Hz, | | (M + H)⁺ |
| | 0.7H), 8.89 (d, J = 8.0 Hz, 1H), 8.77 (t, J = 5.6 Hz, 0.3H), 8.53 (t, | | |
| | J = 5.8 Hz, 0.7H), 8.30 (s, 0.3H), 7.95-8.03 (m, 1H), 7.77 (s, 0.7H), | | |
| | 7.60-7.68 (m, 2H), 7.25-7.34 (m, 2H), 5.06 (s, 2H), 4.97 (dt, J = 8.0, | | |
| | 4.8 Hz, 1H), 4.48-4.76 (m, 2H), 4.32 (qd, J = 7.0, 1.3 Hz, 2H), | | |
| | 3.47-3.61 (m, 1H), 2.78-2.99 (m, 2H), 2.54-2.64 (m, 1H), 2.16 (s, 3H), | | |
| | 1.46 (t, J = 6.9 Hz, 3H), 1.33-1.60 (m, 4H), 1.05-1.28 (m, 6H), | | |
| | 0.66-0.97 (m, 6H). | | |
| 205 | ¹H NMR (DMSO-d₆) (Rotamers present in NMR) d: 12.96 (br. s, | 1.05^{k} | 845.5 |
| | 1H), 12.56 (br. s, 1H), 8.80-9.14 (m, 1H), 8.86 (d, J = 7.6 Hz, 1H), | | (M + H)⁺ |
| | 8.56-8.78 (m, 1H), 8.08-8.31 (m, 1H), 7.98 (d, J = 7.6 Hz, 1H), | | |
| | 7.93-8.07 (m, 1H), 7.59 (d, J = 8.4 Hz, 1H), 7.57 (s, 1H), 7.28-7.33 (m, | | |
| | 1H), 7.23-7.28 (m, 2H), 6.90-7.06 (m, 0.7H), 6.49-6.61 (m, 0.3H), | | |
| | 4.75-4.87 (m, 1H), 4.67 (quin, J = 6.4 Hz, 1H), 4.57 (quin, J = 6.4 Hz, | | |
| | 1H), 4.28 (q, J = 7.2 Hz, 2H), 3.91-4.04 (m, 1H), 3.82 (s, 3H), | | |
| | 2.90 (dd, J = 16.6, 5.6 Hz, 1H), 2.79 (dd, J = 16.6, 5.4 Hz, 1H), | | |
| | 2.61-2.71 (m, 1H), 1.61-1.76 (m, 1H), 1.46 (t, J = 7.2 Hz, 3H), 1.32-1.57 (m, | | |
| | 2H), 1.04-1.30 (m, 6H), 0.80-1.02 (m, 4H), 0.72 (t, J = 6.8 Hz, 3H). | | |
| 206 | ¹H NMR (DMSO-d₆) (Rotamers present in NMR) d: 12.91 (br. s, 1H), | 1.04^{k} | 801.6 |
| | 12.51 (br. s, 1H), 8.79-9.10 (m, 1H), 8.86 (d, J = 7.2 Hz, 1H), | | (M + H)⁺ |
| | 8.56-8.78 (m, 1H), 8.08-8.32 (m, 1H), 7.91-8.07 (m, 1H), 7.98 (d, J = 7.6 Hz, | | |
| | 1H), 7.59 (d, J = 8.4 Hz, 1H), 7.56 (s, 1H), 7.18-7.31 (m, 2H), | | |
| | 7.09-7.17 (m, 1H), 6.85-7.00 (m, 1H), 4.76-4.86 (m, 1H), 4.68 (quin, | | |
| | J = 6.4 Hz, 1H), 4.57 (quin, J = 6.2 Hz, 1H), 4.28 (q, J = 7.0 Hz, 2H), | | |
| | 3.87-4.06 (m, 1H), 3.83 (s, 3H), 2.90 (dd, J = 16.8, 5.4 Hz, 1H), | | |
| | 2.79 (dd, J = 16.8, 5.4 Hz, 1H), 2.61-2.72 (m, 1H), 1.56-1.79 (m, 1H), | | |
| | 1.46 (t, J = 6.9 Hz, 3H), 1.33-1.55 (m, 2H), 1.04-1.32 (m, 7H), | | |
| | 0.81-1.03 (m, 3H), 0.72 (t, J = 6.8 Hz, 3H). | | |
| 207 | ¹H NMR (DMSO-d₆) d: 12.94 (br. s, 0.7H), 12.55 (br. s, 0.6H), | 0.96^{k} | 766.8 |
| | 8.88 (d, J = 7.8 Hz, 2.7H), 8.03-8.23 (m, 1H), 7.99 (d, J = 8.3 Hz, 1H), | | (M + H)⁺ |
| | 7.72-7.80 (m, 2H), 7.55-7.63 (m, 2H), 7.23-7.30 (m, 2H), 6.72 (br. s, | | |
| | 0.7H), 6.48-6.56 (m, 2H), 4.79-4.85 (m, 1H), 4.55-4.74 (m, 2H), | | |
| | 4.29 (q, J = 7.2 Hz, 2.2H), 3.92 (br. s, 0.8H), 2.76-2.95 (m, 2H), 2.72 (s, | | |
| | 3H), 2.68 (br. s, 0.7H), 1.65 (br. s, 0.9H), 1.47 (t, J = 6.9 Hz, 6H), | | |
| | 1.08-1.31 (m, 6H), 0.86-0.99 (m, 3H), 0.73 (t, J = 7.0 Hz, 3H). | | |
| 208 | ¹H NMR (DMSO-d₆) d: 12.74-13.35 (m, 1H), 12.21-12.73 (m, 0.9H), | 0.90^{k} | 752.8 |
| | 8.57-9.18 (m, 3H), 8.19 (s, 0.1H), 8.05 (s, 0.8H), 7.99 (d, J = 8.0 Hz, | | (M + H)⁺ |
| | 1H), 7.65-7.77 (m, 2H), 7.55-7.64 (m, 2H), 7.21-7.29 (m, 2H), | | |
| | 6.52-6.63 (m, 2H), 6.17 (br. s, 1.4H), 4.82 (dt, J = 7.8, 4.9 Hz, 1H), | | |
| | 4.53-4.75 (m, 2H), 4.36-4.47 (m, 0.2H), 4.30 (q, J = 7.0 Hz, 2H), 3.91 (br. | | |
| | s, 0.9H), 2.75-2.98 (m, 2H), 2.68 (br. s, 1H), 1.59-1.71 (m, 1H), | | |
| | 1.28-1.56 (m, 6H), 1.07-1.26 (m, 6H), 0.91-1.00 (m, 3H), 0.72 (t, | | |
| | J = 7.0 Hz, 3H). | | |
| 209 | ¹H NMR (DMSO-d₆) (Rotamers present in NMR) d: 12.15-13.49 (m, | 0.76^{k} | 766.8 |
| | 1.1H), 8.72-9.09 (m, 3H), 7.92-8.49 (2H, broad peak underneath | | (M + H)⁺ |
| | several peaks), 8.15 (br. s., 1H), 8.08 (d, J = 7.3 Hz, 2H), | | |
| | 7.97-8.04 (m, 1H), 7.56-7.65 (m, 4H), 7.22-7.32 (m, 2H), 4.62-4.80 (m, 2H), | | |
| | 4.51-4.61 (m, 1H), 4.26-4.35 (m, 2.3H), 4.14 (s, 2H), 4.02 (br. s., | | |
| | 0.8H), 2.65-2.92 (m, 3H), 1.30-1.76 (m, 7H), 1.09-1.27 (m, 6H), | | |
| | 0.96 (t, J = 6.9 Hz, 3H), 0.73 (t, J = 6.9 Hz, 3H). | | |
| 300 | ¹H NMR (DMSO-d₆) (Rotamers present in NMR) d: 9.70 (br. s, | 6.63^{a} | 654.3 |
| | 0.3H), 9.36 (br. s, 0.7H), 9.13-9.19 (m, 0.3H), 9.04 (t, J = 5.9 Hz, | | (M + H)⁺ |
| | 0.7H), 8.78 (t, J = 5.5 Hz, 0.3H), 8.53 (t, J = 5.8 Hz, 0.7H), 8.29 (s, | | |
| | 0.3H), 7.76 (s, 0.7H), 7.66-7.73 (m, 1H), 7.63-7.66 (m, 0.7H), | | |
| | 7.60-7.63 (m, 0.3H), 7.16-7.28 (m, 2H), 7.10-7.16 (m, 1H), 5.55 (d, | | |
| | J = 13.6 Hz, 2H), 4.45-4.73 (m, 2H), 4.12 (q, J = 6.8 Hz, 2H), 3.57 (td, | | |
| | J = 9.3, 5.0 Hz, 1H), 2.55-2.64 (m, 1H), 2.27-2.37 (m, 1H), | | |
| | 1.44-1.57 (m, 2H), 1.34-1.40 (m, 5H), 1.05-1.22 (m, 6H), 0.90 (d, J = 7.0 Hz, | | |
| | 6H), 0.65-0.81 (m, 6H). | | |
| 301 | ¹H NMR (DMSO-d₆) (Rotamers present in NMR) d: 9.63 (s, 0.3H), | 6.37^{a} | 666.3 |
| | 9.32 (br. s, 0.7H), 9.22 (t, J = 6.0 Hz, 0.3H), 9.07 (t, J = 6.0 Hz, 0.7H), | | (M + H)⁺ |
| | 8.78 (t, J = 5.8 Hz, 0.3H), 8.52 (t, J = 5.8 Hz, 0.7H), 8.29 (s, 0.3H), | | |
| | 7.76 (s, 0.7H), 7.71-7.73 (m, 0.6H), 7.66-7.71 (m, 1.4H), | | |
| | 7.20-7.28 (m, 2H), 7.09-7.17 (m, 1H), 4.79 (d, J = 9.8 Hz, 2H), 4.47-4.73 (m, | | |
| | 2H), 4.14 (q, J = 7.0 Hz, 2H), 3.57 (td, J = 9.5, 4.6 Hz, 1H), | | |
| | 2.54-2.64 (m, 1H), 2.05 (s, 3H), 1.44-1.58 (m, 2H), 1.38 (t, J = 7.0 Hz, 3H), | | |
| | 1.33-1.43 (m, 2H), 1.06-1.22 (m, 6H), 0.67-0.82 (m, 6H). | | |
| 302 | ¹H NMR (DMSO-d₆) (Rotamers present in NMR) d: 9.65 (s, 0.3H), | 6.49^{a} | 670.3 |
| | 9.32 (br. s, 0.7H), 9.17-9.22 (m, 0.3H), 9.05 (t, J = 5.9 Hz, 0.7H), | | (M + H)⁺ |
| | 8.74-8.81 (m, 0.3H), 8.52 (t, J = 5.8 Hz, 0.7H), 8.29 (s, 0.3H), 7.76 (s, | | |
| | 0.7H), 7.70-7.73 (m, 0.6H), 7.66-7.69 (m, 1.4H), 7.18-7.28 (m, 2H), | | |
| | 7.10-7.17 (m, 1H), 5.56 (d, J = 13.6 Hz, 2H), 4.47-4.76 (m, 3H), | | |
| | 4.13 (q, J = 6.8 Hz, 2H), 3.57 (td, J = 9.3, 5.4 Hz, 1H), 2.54-2.65 (m, 1H), | | |
| | 1.45-1.58 (m, 2H), 1.38 (t, J = 7.0 Hz, 3H), 1.33-1.44 (m, 2H), | | |
| | 1.13 (d, J = 6.3 Hz, 6H), 1.08-1.22 (m, 6H), 0.68-0.81 (m, 6H). | | |
| 400 | ¹H NMR (DMSO-d₆) (Rotamers present in NMR) d: 10.12 (br. s, | 6.73^{a} | 630.3 |
| | 0.3H), 9.71 (br. s, 0.7H), 8.73-8.87 (m, 1.3H), 8.56 (t, J = 5.8 Hz, | | (M + H)⁺ |
| | 0.7H), 8.27 (s, 0.3H), 7.81 (d, J = 12.5 Hz, 1H), 7.75 (s, 0.7H), | | |
| | 7.47 (br. s, 1H), 7.12-7.24 (m, 4H), 7.02-7.11 (m, 3H), 6.89-6.97 (m, 1H), | | |
| | 4.50-4.71 (m, 2H), 4.03 (q, J = 7.0 Hz, 2H), 3.50-3.59 (m, 1H), | | |
| | 2.53-2.66 (m, 1H), 1.47-1.60 (m, 2H), 1.35-1.45 (m, 2H), 1.32 (t, J = 6.9 Hz, | | |
| | 3H), 1.06-1.23 (m, 6H), 0.68-0.83 (m, 6H). | | |
| 500 | ¹H NMR (DMSO-d₆) (Rotamers present in NMR) d: 12.93 (br. s, | 1.04^{k} | 796.7 |
| | 1H), 12.53 (br. s, 1H), 8.80-9.15 (m, 1H), 8.86 (d, J = 7.8 Hz, 1H), | | (M + H)⁺ |
| | 8.57-8.79 (m, 1H), 8.16-8.47 (m, 1H), 8.18 (d, J = 8.8 Hz, 1H), | | |
| | 8.09-8.15 (m, 1H), 8.02-8.09 (m, 1H), 7.97 (d, J = 8.3 Hz, 1H), | | |
| | 7.50-7.63 (m, 2H), 7.15-7.28 (m, 2H), 4.75-4.86 (m, 1H), 4.67 (quin, J = 6.3 Hz, | | |
| | 1H), 4.57 (quin, J = 6.4 Hz, 1H), 4.28 (q, J = 7.0 Hz, 2H), 3.97-4.13 (m, | | |
| | 1H), 2.91 (dd, J = 16.6, 5.0 Hz, 1H), 2.79 (dd, J = 17.0, 5.0 Hz, 1H), | | |
| | 2.61-2.71 (m, 1H), 2.52 (s, 3H), 1.65-1.79 (m, 1H), 1.46 (t, J = 6.9 Hz, | | |
| | 3H), 1.35-1.60 (m, 3H), 1.07-1.32 (m, 6H), 0.84-1.03 (m, 3H), | | |
| | 0.73 (t, J = 6.8 Hz, 3H). | | |
| 600 | ¹H NMR (DMSO-d₆) (Rotamers present in NMR) d: 12.97 (br. s, | 0.89^{k} | 752.6 |
| | 1H), 12.55 (br. s, 1H), 9.00 (t, J = 6.0 Hz, 1H), 8.87 (d, J = 7.8 Hz, 1H), | | (M + H)⁺ |
| | 8.65-8.80 (m, 1H), 8.11 (br. s, 1H), 7.99 (d, J = 8.3 Hz, 1H), | | |
| | 7.58-7.65 (m, 2H), 7.23-7.31 (m, 2H), 7.12-7.23 (m, 3H), 6.87 (d, J = 7.0 Hz, | | |
| | 1H), 4.82 (dt, J = 7.8, 4.8 Hz, 1H), 4.52-4.75 (m, 2H), 4.30 (q, | | |
| | J = 7.0 Hz, 2H), 3.91-4.06 (m, 1H), 2.74-2.96 (m, 2H), 2.58-2.70 (m, | | |
| | 1H), 1.60-1.76 (m, 1H), 1.47 (t, J = 6.9 Hz, 3H), 1.32-1.58 (m, 2H), | | |
| | 1.03-1.29 (m, 7H), 0.95 (t, J = 7.3 Hz, 3H), 0.72 (t, J = 6.9 Hz, 3H). | | |
| 601 | ¹H NMR (DMSO-d₆) (Rotamers present in NMR) d: 12.88 (br. s, | 1.00^{k} | 780.7 |
| | 2H), 8.88 (d, J = 7.8 Hz, 1H), 8.81 (br. s, 1H), 8.14 (s, 1H), 7.98 (d, | | (M + H)⁺ |
| | J = 8.0 Hz, 1H), 7.51-7.63 (m, 2H), 7.36 (br. s, 1H), 7.19-7.32 (m, | | |
| | 4H), 7.15 (br. s, 1H), 7.01 (d, J = 6.8 Hz, 1H), 4.82 (dt, J = 7.8, 4.9 Hz, | | |
| | 1H), 4.53-4.77 (m, 2H), 4.29 (q, J = 6.9 Hz, 2H), 3.96-4.03 (m, 1H), | | |
| | 2.89 (s, 6H), 2.76-2.96 (m, 2H), 2.61-2.74 (m, 1H), 1.63-1.77 (m, | | |
| | 1H), 1.47 (t, J = 7.0 Hz, 3H), 1.35-1.58 (m, 2H), 1.05-1.28 (m, 7H), | | |
| | 0.91-1.01 (m, 3H), 0.73 (t, J = 6.8 Hz, 3H). | | |
| 602 | ¹H NMR (DMSO-d₆) (Rotamers present in NMR) d: 12.96 (br. s, | 1.88^{b} | 766.3 |
| | 1H), 12.57 (br. s, 1H), 8.94 (br. s, 1H), 8.88 (d, J = 7.8 Hz, 1H), | | (M + H)⁺ |
| | 8.71-8.82 (m, 1H), 8.13 (br. s, 1H), 7.95-8.07 (m, 1H), 7.53-7.66 (m, 2H), | | |
| | 7.16-7.34 (m, 4H), 7.08 (br. s, 1H), 6.78-6.89 (m, 1H), 4.77-4.87 (m, | | |
| | J = 5.3 Hz, 1H), 4.51-4.77 (m, 2H), 4.21-4.37 (m, 2H), 3.92-4.06 (m, | | |
| | 1H), 2.74-2.99 (m, 2H), 2.68 (br. s, 3H), 2.54-2.59 (m, 1H), | | |
| | 1.63-1.76 (m, 1H), 1.39-1.52 (m, 3H), 1.32-1.59 (m, 2H), 1.06-1.27 (m, | | |
| | 7H), 0.96 (t, J = 6.3 Hz, 3H), 0.72 (t, J = 5.4 Hz, 3H). | | |

Analytical methods:
^{a}LCMS Method: Agilent 1100 Series LC/MSD SL or VL using electrospray positive [ES+ ve to give M + H⁺] equipped with a Xorbax Eclipse XDB-C8 5.0 μm column (4.6 mm × 150 mm, i.d.), eluting with 0.05% TFA in water (solvent A) and 0.05% TFA in CH₃CN (solvent B), using the following elution gradient 10-100% (solvent B) over 10.0 min and holding at 100% for 1.6 min at a flow rate of 1.0 mL/min.
^{b}LCMS Method: Agilent 1100 Series LC/MSD SL or VL using electrospray positive [ES+ ve to give M + H⁺] equipped with a Sunfire C18 5.0 μm column (3.0 mm × 50 mm, i.d.), eluting with 0.05% TFA in water (solvent A) and 0.05% TFA in CH₃CN (solvent B), using the following elution gradient: 10-100% (solvent B) over 2.5 min and holding at 100% for 1.7 min at a flow rate of 1.0 mL/min.
^{c}LCMS Method: Agilent 1200 Series LC/MSD SL or VL using electrospray positive [ES+ ve to give M + H⁺] equipped with a Sunfire C18 5.0 μm column (3.0 mm × 50 mm, i.d.), eluting with 0.1% TFA in water (solvent A) and 0.1% TFA in CH₃CN (solvent B), using the following elution gradient: 10-100% (solvent B) over 2.5 min and holding at 100% for 1.7 min at a flow rate of 1.0 mL/min.
^{d}UPLC Method: Acquity UPLC with SQD MSD using electrospray positive [ES+ ve to give M + H⁺] equipped with a BEH C18 1.7 μm column (2.1 mm × 50 mm i.d.) eluting with 0.1% formic acid in water (solvent A) and 0.1% formic acid in CH₃CN (solvent B), using the following elution gradient: 3-100% (solvent B) over 1.5 min and holding at 100% for 0.4 min at a flow rate of 1.0 mL/min.
^{e}LCMS Method: Shimadzu 10Avp with Sedere Sedex 75C and PE Sciex Single Quadrupole 150EX using electrospray positive [ES+ ve to give M + H⁺] equipped with a Thermo Hypersil Gold C18 1.9 μm column (2.1 mm × 20 mm i.d.) eluting with 0.02% TFA in water (solvent A) and 0.02% TFA in CH₃CN (solvent B), using the following elution gradient: 4-95% (solvent B) over 1.88 min and holding at 4% for 0.9 min at a flow rate of 1.4 mL/min.
^{f}LCMS Method: Shimadzu 10Avp with Sedere Sedex 75C and Waters ZQ Single Quadrupole using electrospray positive [ES+ ve to give M + H⁺] equipped with a Thermo Hypersil Gold C18 1.9 μm column (2.1 mm × 20 mm i.d.) eluting with 0.02% TFA in water (solvent A) and 0.02% TFA in CH₃CN (solvent B), using the following elution gradient: 4-95% (solvent B) over 1.88 min and holding at 4% for 0.9 min at a flow rate of 1.4 mL/min.
^{g}LCMS Method: Agilent 1200 Series LC/MSD SL or VL using electrospray positive [ES+ ve to give M + H⁺] equipped with a Zorbax C18 5.0 μm column (4.6 mm × 150 mm, id.), eluting with 0.1% TFA in water (solvent A) and 0.1% TFA in CH₃CN (solvent B), using the following elution gradient: 10-100% (solvent B) over 12.5 min and holding at 100% for 1.8 min at a flow rate of 1.0 mL/min.
^{h}LCMS Method: Agilent 1200 Series LC/MSD SL or VL using electrospray positive [ES+ ve to give M + H⁺] equipped with a Sunfire C18 2.5 μm column (2.1 mm × 20 mm, id.), eluting with 0.05% TFA in water (solvent A) and 0.05% TFA in CH₃CN (solvent B), using the following elution gradient: 10-100% (solvent B) over 2.5 min and holding at 100% for 0.2 min at a flow rate of 1.3 mL/min.
ⁱLCMS Method: Agilent 1200 Series LC/MSD SL or VL using electrospray positive [ES+ ve to give M + H⁺] equipped with an Agilent Eclipse XBD-C18 5.0 μm column (4.6 mm × 250 mm, id.), eluting with 0.05% TFA in water (solvent A) and 0.05% TFA in CH₃CN (solvent B), using the following elution gradient: 1-99% (solvent B) over 10 min at a flow rate of 1.0 mL/min.
^{j}LCMS Method: Agilent 1200 Series LC/MSD SL or VL using electrospray positive [ES+ ve to give M + H⁺] equipped with an Zorbax Eclipse XBD-C18 5.0 μm column (4.6 mm × 150 mm, id.), eluting with 0.1% TFA in water (solvent A) and 0.1% TFA in CH₃CN (solvent B), using the following elution gradient: 10-100% (solvent B) over 12 min and holding at 100% for 1 min at a flow rate of 1.0 mL/min.
^{k}UPLC Method: Acquity UPLC with SQD MSD using electrospray positive [ES+ ve to give M + H⁺] equipped with a Thermo Hypersil Gold C18 1.9 μm, (20 mm × 2.1 mm id.) eluting with 0.02% TFA in water (solvent A) and 0.02% TFA in CH₃CN (solvent B), using the following elution gradient: 0.5-98% (solvent B) over 1.9 min at a flow rate of 1.6 mL/min.

### Pharmaceutical Compositions

Example A—Tablets are prepared using conventional methods and are formulated as follows:

| Ingredient | Amount per tablet |
|---|---|
| Compound of the invention | 5 mg |
| Microcrystalline cellulose | 100 mg |
| Lactose | 100 mg |
| Sodium starch glycollate | 30 mg |
| Magnesium stearate | 2 mg |
| Total | 237 mg |

Example B—Capsules are prepared using conventional methods and are formulated as follows:

| Ingredient | Amount per tablet |
|---|---|
| Compound of the invention | 15 mg |
| Dried starch | 178 mg |
| Magnesium stearate | 2 mg |
| Total | 195 mg |

Example C—Nanosuspensions and micron-sized suspensions are prepared using conventional aqueous milling technology such as bead milling methods and are formulated as follows:

| Ingredient | Amount per nanosuspension |
|---|---|
| Compound of the invention | 50 mg |
| Polysorbate 20 | 10 mg |
| Polyethylene Glycol 4000 | 20 mg |
| Mannitol | 30 mg |
| Purified Water | qs |
| Total | 110 mg |

Example D—Melt Extrudates are prepared using conventional melt extrusion techniques and cryomilling to achieve adequate particle size as follows:

| Ingredient | Amount per Melt Extrudate |
|---|---|
| Compound of the invention | 67 mg |
| 75:25 Poly(lactic-co-glycolic acid) | 34 mg |
| Total | 100 mg |

Example E—A lyophilized product is prepared by conventional methods formulated as follows:

| Ingredient | Amount per Lyophilized Formulation |
|---|---|
| Compound of the invention | 20 mg |
| Sodium Hydroxide | qs |
| Glycine | 30 mg |
| Polyethylene Glycol | 50 mg |
| Polysorbate | 2.5 mg |

Example F—A solution for injection product is prepared by conventional methods formulated as follows:

| Ingredient | Amount |
|---|---|
| Compound of the invention | 10 mg |
| Sodium Hydroxide | qs to pH adjust |
| Hydrochloric acid | qs to pH adjust |
| Sodium Chloride or Mannitol | qs to adjust tonicity |
| Polyethylene Glycol | qs to solubilize |
| Cyclodextrin | qs to solubilize |
| Tromethamine (or other buffer) | qs |
| Water for injection | qs to volume (concentration) |

### Biological Assays

Compounds, including compounds of the invention, may be tested for BMP-1/TLL1/TLL2 inhibition activity according to the enzyme inhibition assays below.

Compounds, including compounds of the invention, may be tested for inhibition of the processing of procollagen substrate by native enzyme produced by the fibroblast (the cell type that drives fibrosis in vivo), according to the cellular assay below.

### Materials:

Buffer components are purchased from Sigma-Aldrich (St. Louis, Mo.) or an equivalent supplier. The promyostatin peptide substrate is custom synthesized by American Peptide Company (Sunnyvale, Calif.) using the myostatin protein sequence (Uniprot accession number 014793) surrounding the cleavage site reviewed in Hopkins, D. R., et al., 2007 Matrix Biology, 26, 508-523. The procollagen peptide substrate used in the high enzyme BMP1 cleavage assay is custom synthesized by 21^{st} Century Biochemicals (Marlboro, Mass.) using the procollagen Iα protein sequence (Uniprot accession number P02452) surrounding the cleavage site reviewed in Hopkins, D. R., et al., 2007 Matrix Biology, 26, 508-523.

### Preparation of Human BMP1 Protein:

The DNA sequence encoding amino acids 23-721 of human BMP1 (NM_001199.3) with the human RAGE signal sequence (aa1-22 of NM_001136) at the N-terminus and FLAG-6×His epitope tags at the C-terminus is amplified using PCR technology. The resultant Rgss-BMP1(23-721)-FLAG-6×His fragment is subcloned into pCDN, a mammalian expression vector driven by the CMV promoter and containing the DHFR gene to allow selection in nucleoside-free cell culture media. This construct is electroporated into CHOE1a cells. After selection, conditioned media from individual clones are analyzed using a BMP1 assay for promyostatin-derived peptidase activity (see assay below). Conditioned media from several clones with the highest activity are analyzed via western blot to confirm expression. The clone with the highest expression and peptidase activity is used for protein expression.

The mature form of human BMP1 (121-721), secreted from the stably transfected CHO cell line, is purified. All purification steps are carried out at 4° C. 10 l of conditioned medium is concentrated to 1.2 l with a Watson Marlow diafiltration system (A/G Technology Corporation, Model # UFP-10-C-55) using a 10 kDa cut off cartridge. A subsequent buffer exchange is carried out on the same system with 5 l of 50 mM Tris buffer, pH 8.0, containing 0.5 M NaCl, 20% glycerol, 1 mM CHAPS, 5 mM CaCl₂, 10 μM ZnCl₂, and 20 mM imidazole. The diafiltrated medium is subjected to successive nickel NTA superflow chromatography (Qiagen, Valencia, Calif.) using 50 ml, 30 ml, and 15 ml resin volumes, each overnight at 4° C., and the unbound fraction containing most of the BMP1 is retained. 100 ml of this unbound fraction is diluted into 1000 ml of 50 mM Tris buffer, pH 8.0, containing 20% glycerol, 10 mM NaCl, 5 mM CaCl₂, 10 μM ZnCl₂, and 1 mM CHAPS and applied to 20 ml of Q Sepharose Fast Flow (GE Healthcare Life Sciences). The Q Sepharose unbound fraction, which contains BMP1, is further concentrated on a Viva Spin, 10 kDa cut off cartridge (Viviproducts, Littleton, Mass.).

### Preparation of Human TLL1 Protein:

The DNA sequence encoding a natural variant of full length native human TLL1 (NM_012464.4) containing three amino acid substitutions I156V, N221S, V284A is amplified from human heart and brain cDNA and subcloned into the pCDN expression vector. The plasmid is electroporated into CHOE1A cells. After selection, a clone expressing high levels of TLL1 is scaled and used for protein purification.

All purification steps are carried out at 4° C. CHO conditioned medium was diluted 3-fold with 5 mM Tris buffer, pH 8.4, and human TLL1 is captured by Source 30 Q resin (GE Healthcare Life Sciences). After an extensive wash with 50 mM Tris buffer, pH 8.0, human TLL1 is eluted with a linear gradient of 0 to 0.5 M NaCl in 50 mM Tris buffer, pH 8.0. Following a 3.6-fold dilution into 20 mM Tris buffer, pH 7.4, human TLL1 from the Source 30Q pool is then captured onto a Macro-prep ceramic hydroxyapatite (HA) type I 40 μm resin (BioRad, Hercules, Calif.). The HA resin is washed with 20 mM Tris buffer, pH 7.4, and human TLL1 is eluted with 0.5 M potassium phosphate buffer, pH 7.4, in a linear gradient from wash buffer. Human TLL1 from the HA pool was salt fractionated with 40% ammonium sulfate saturation and resolubilized with 20 mM Tris buffer, pH 7.0, containing 0.25 M NaCl and 7 mM CaCl₂.

### Preparation of Human TLL2 Protein:

The DNA sequence encoding amino acids 26-1015 of human TLL2 (NM_012465) is PCR amplified from DNA template with the human RAGE signal sequence at the N-terminus and Avi-6×His epitope tags at the C-terminus (GGLNDIFEAQKIEWHEHHHHHH). The Rgss-TLL2 Avi-6×His fragment is subcloned into a pCDN expression vector by Gateway™ recombination (Life Technologies, Grand Island, N.Y.). DHFR deficient CHOE1a cells are maintained in MR1 media (Life Technologies) supplemented with nucleosides at 37° C. in 5% CO2. Linearized plasmid DNA is electroporated into the cells and clones are generated in media without nucleosides. Clones are screened for TLL2 activity in the promyostatin-derived peptidase assay (see below) which allowed identification of clones that expressed optimal levels of the active form of TLL2.

Stably expressing TLL2 CHO cell conditioned medium is concentrated by diafiltration as described for BMP1. 325 ml of concentrated medium is purified by nickel NTA superflow chromatography (20 ml Ni-NTA SF, overnight at 4° C.). The resin is washed with a 15 mM to 100 mM imidazole linear gradient, and protein is eluted with 0.3 M imidazole in buffer A (50 mM Tris, pH 8.0, 0.5 M NaCl, 20% glycerol, 1 mM CHAPS, 5 mM CaCl₂, 10 μM ZnCl₂).

### Enzyme Inhibition Assay for Human BMP1:

(i) Low Enzyme Concentration

Inhibition of BMP1 peptidase activity by test compounds of the invention is measured by monitoring cleavage of a promyostatin peptide substrate by recombinant, mature BMP1 protein (BMP1(121-721)-Flag-His). FRET quenching of dual-labeled peptide ((5-FAM)-ELIDQYDVQRDDSSDGSLED-K(5,6 TAMRA)-CONH₂) is relieved by BMP1-catalyzed cleavage. This assay is run as a 10 μl endpoint assay in 384-well format where the reaction contains 0.5 nM BMP1 and 0.8 μM promyostatin peptide substrate in 25 mM HEPES buffer, pH 7.5, containing 0.01% Brij-35 detergent, 5 mM CaCl₂, and 1 μM ZnCl₂. The assay is run by adding 5 μl enzyme solution to a black, low volume assay plate (Greiner 784076) pre-dispensed with 100 nl test compound solutions in DMSO. After 10 mins, 5 μl substrate are added and the reaction is incubated at ambient temperature for an additional 60 mins. The reaction is quenched with 5 μl of 0.5 M EDTA and the plate is read on a ViewLux (PerkinElmer) multilabel plate reader using a 480 nm excitation filter and 540 nm emission filter. The test compounds are prepared in neat DMSO at a concentration of 10 mM. For inhibition curves, compounds are diluted in DMSO using a three-fold serial dilution and tested at 11 concentrations (100 μM-1.7 nM, final 1% DMSO). Responses are normalized to the uninhibited and no-enzyme controls within each plate. Dose-response curves are analyzed using a four-parameter logistic fit in ActivityBase and results are expressed as pIC₅₀ values.

The compounds of PCT application no. PCT/IB2015/050179 and PCT publication no. WO2015/104684 Examples 1-115 and 117-149 were tested and exhibited a pIC₅₀>6.9 according to this assay.

(ii) High Enzyme Concentration

Use of a high enzyme concentration assay may be useful, e.g., as discussed in Habig, M., et al., Journal of Biomolecular Screening, 2009, 14, 679-689.

This assay is run as a 10 μl endpoint assay in 384-well format where the reaction contains 50 nM BMP1 enzyme and 6 μM procollagen I peptide substrate ((5-FAM)-DGGRYYRADDANVVRD-K(5,6-TAMRA)-CONH₂) in 25 mM HEPES buffer, pH 7.5, containing 0.01% Brij-35 detergent, 5 mM CaCl₂, and 1 μM ZnCl₂. The assay is run by adding 5 μl enzyme solution to a black, low volume assay plate (Greiner 784076) pre-dispensed with 100 nl test compound solutions in DMSO. After 10 mins, 5 μl substrate are added and the reaction is incubated at ambient temperature for an additional 30 mins. The reaction is quenched with 5 μl of 0.5 M EDTA and the plate is read on a ViewLux (Perkin Elmer) multilabel plate reader using a 480 nm excitation filter and 540 nm emission filter. Data fitting and compound preparations are performed as described above for the low enzyme concentration.

The compounds of PCT application no. PCT/IB2015/050179 and PCT publication no. WO2015/104684 Examples 1-115, 117-146 and 149 were tested and exhibited a pIC₅₀>6.7 according to this assay.

The compounds of Examples 100-101, 104, 108, 112-116, 142-143, 302, and 400 of the present invention were tested in both the low enzyme and high enzyme concentration inhibition assays and exhibited a pIC₅₀>6.5 according to both assays.

### Enzyme Inhibition Assay for Human TLL1 and TLL2:

Inhibition of human TLL1 and TLL2 recombinant enzymes is measured in 10 ul endpoint assays in 384-well format using the same promyostatin peptide substrate employed in the above Enzyme Inhibition Assay for human BMP1. The TLL1 reaction contains 2 nM TLL1 and 0.8 μM promyostatin peptide substrate in 25 mM HEPES buffer, pH 7.5, containing 0.01% Brij-35 detergent, 5 mM CaCl₂, and 1 μM ZnCl₂. The TLL1 assay is run by adding 5 μl enzyme solution to a black, low volume assay plate (Greiner 784076) pre-dispensed with 100 nl test compound solutions in DMSO. Following a 10 minute preincubation of enzyme with inhibitor, 5 μl of substrate solution are added. TLL1 reactions are incubated at ambient temperature for an additional 60 minutes. The TLL2 reaction contains 18 nM TLL2 and 5 μM promyostatin peptide substrate in 25 mM HEPES buffer, pH 7.5, containing 0.01% Brij-35 detergent, 5 mM CaCl₂, and 1 μM ZnCl₂. The TLL2 assay is run without an enzyme-inhibitor preincubation by adding 5 μl enzyme and 5 μl substrate solutions to a black, low volume assay plate (Greiner 784076) pre-dispensed with 100 nl compound solutions in DMSO. TLL2 reactions are incubated at ambient temperature for 60 minutes. TLL1 and TLL2 reactions are quenched with 5 μl of 0.5 M EDTA and plates are read on a ViewLux (Perkin Elmer) multilabel plate reader using a 480 nm excitation filter and 540 nm emission filter. Data fitting and compound preparations are performed as described above for the Enzyme Inhibition Assay for human BMP1.

The compounds of PCT application no. PCT/IB2015/050179 and PCT publication no. WO2015/104684 Examples 1-33, 35-71, 73-84, 86-115, 117-140, and 142-146 were tested in the TLL1 enzyme inhibition assay and exhibited a pIC₅₀>6.4 according to this assay.

The compounds of PCT application no. PCT/IB2015/050179 and PCT publication no. WO2015/104684 Examples 1-24, 26-33, 35-71, 73-78, 80-84, 86-115, 117-140, and 142-146 were tested in the TLL2 enzyme inhibition assay and exhibited a pIC₅₀>6.1 according to this assay.

The enzyme assay results indicate that the tested compounds of PCT application no. PCT/IB2015/050179 and PCT publication no. WO2015/104684 are potent inhibitors of one or more of BMP1, TLL1 and TLL2 enzymatic activity. The tested compounds inhibited one or more of these metalloproteases in biochemical assays using isolated enzymes and peptide substrates.

The compounds of Examples 100-101, 104, 112-116, 142-143, 302, and 4000f the present invention were tested in the TLL1 enzyme inhibition assay and exhibited a pIC₅₀>6.6 according to this assay.

The compounds of Examples 100-101, 104, 112-116, 142-143, 302, and 400 of the present invention were tested in the TLL2 enzyme inhibition assay and exhibited a pIC₅₀>6.4 according to this assay.

### Cell-Based Inhibition Assay of Generation of Procollagen I C-Terminal Propeptide (PICP) and Mature Collagen:

An adaptation of the collagen deposition assay described by Chen, C. Z. C., et al., British Journal of Pharmacology, 2009, 158, 1196-1209 is used to examine effect of compounds on procollagen I processing and collagen deposition. In the adapted assay, human cardiac fibroblasts are utilized. Processing of procollagen I is determined by a PICP ELISA assay and deposition of mature collagen is determined by immunostaining.

Human cardiac fibroblasts are cultured and maintained until passage 6 in FGM-3 media (Lonza, #CC-3132) in a 37° C. humidified incubator with 5% CO₂. They are then seeded in 96-well black wall, clear bottom plates at 10,000 to 15,000 cells per well in eagle's minimum essential media (EMEM, ATCC #30-2003) containing 10% fetal bovine serum (FBS, Life Technologies #10082147), 1% Glutamax (Life Technologies #35050061) and 1% Penicillin and Streptomycin (Life Technologies #15070063). These cultures are placed in 37° C. incubator. The next day, media is removed by aspiration and cells are rinsed with phosphate buffered saline. Crowding media (also called ficoll media) is prepared by adding 112.5 mg/ml of ficoll70 and 75 mg/ml ficoll400 (GE healthcare #17-0310-10 and 17-0300-10, respectively), 100 μM ascorbic acid, 1% Glutamax and 1% Penicillin and Streptomycin to EMEM media. Test compounds (dissolved in DMSO) are diluted into crowding media and then added to the cells. Final concentration of DMSO in crowding media is less than 0.3%. Cells are treated for 24 to 48 hr in a 37° C. incubator. At the end of the treatment period, cell media are collected. The level of PICP in the media is determined by a PICP ELISA assay (Quidel #8003) following the manufacturer's protocol. Potencies of test compounds are calculated by fitting PICP levels, relative to untreated controls, to log (inhibitor) vs. response equation using Graphpad Prism software 5.0 and expressed as pIC₅₀.

For some compounds, deposition of mature collagen is measured by immunostaining in addition to PICP levels. At the end of the treatment period, cells on culture plate are fixed with 100% methanol (prechilled to −20° C.) for 10 min. Then the cells are immunostained with mouse anti-mature collagen I antibody (1:500 dilution, Sigma#C2456), anti-mouse secondary antibody Alexa647 (1:500 dilution, Invitrogen#A21236) and Hoechst (for nuclei, 2 μg/ml, Invitrogen#H3596). Fluorescent image acquisition is done using the Operetta High Content Imaging system (Perkin Elmer). For each image field, the intensity of mature collagen staining is normalized with the number of nuclei. Normalized collagen levels are used to calculate the potency of test compounds with Graphpad Prism software, as described above.

The compounds of PCT application no. PCT/IB2015/050179 and PCT publication no. WO2015/104684 Examples 1, 5, 12-14, 16, 18, 22-30, 33-35, 39-45, 47-53, 55, 59-62, 64, 65, 67, 70-78, 80-89, 91-106, 108, 111-115, and 117-146 were tested in the PICP cellular inhibition assay and exhibited a pIC₅₀>5.4 in this assay.

The compounds of PCT application no. PCT/IB2015/050179 and PCT publication no. WO2015/104684 Examples 5, 24, 39, 47, 74, 75, 77, 80-82, 86, 93, 96, 98, 99, 111-113, 121-124, 126-128, 132, and 139 were tested in the mature collagen cellular inhibition assay and exhibited a pIC₅₀>6.0 in this assay.

The above cellular assay results demonstrate that the tested compounds of PCT application no. PCT/IB2015/050179 and PCT publication no. WO2015/104684 inhibit the processing of procollagen substrate by native enzyme produced by the fibroblast, the cell type that drives fibrosis in vivo.

In view of the above biological data, compounds of PCT application no. PCT/IB2015/050179 and PCT publication no. WO2015/104684 should have benefit as antifibrotic agents across a wide variety of diseases driven by pathological fibrosis, and diseases related to other in vivo substrates for these enzymes, e.g., where muscle function or muscle mass is diminished.

The compounds of Examples 100-101, 104, 112-116, 142-143, 302, and 400 of the present invention were tested in the PICP cellular inhibition assay and exhibited a pIC₅₀>6.4 in this assay.

### Human Plasma Stability Assay

A plasma stability assay may be used to determine the ability of a compound of the invention to convert to a BMP1, TLL1 and/or TLL2 inhibitor (for example, a compound disclosed in PCT application no. PCT/IB2015/050179 or PCT publication no. WO2015/104684), in plasma.

Heparinized, pooled male human plasma is obtained from a commercial source (BioreclamationIVT, Baltimore, Md.). Plasma is stored at or about −80° C. until used, and is stored in aliquots to avoid multiple freeze/thaw cycles. Test compound stock solutions are prepared in DMSO. Thawed plasma is centrifuged to remove debris; the pH is adjusted to approximately 7.5 with 1% aqueous phosphoric acid, if necessary. Plasma is prewarmed in a 37° C. water bath for approximately 5 minutes. Incubation mixtures are prepared by adding an aliquot of the test compound DMSO stock solution to the prewarmed plasma. The final test compound incubation concentration ranges from 100 to 2000 ng/mL, and the final DMSO concentration in the incubation mixtures is <0.4%, e.g., 0.2%. Incubations are vortexed to ensure complete mixing, and are incubated in a 37° C. water bath with gentle shaking. Samples (30 uL) are collected at various predetermined time points following addition of the test compounds, typically up to either 4 or 24 hours. Proteins are precipitated immediately following collection of each sample by the addition of 120 uL of acetonitrile or 150 uL methanol containing an appropriate analytical internal standard. Samples are stored at or about −80° C. until analyzed. Concentrations of both the test compound as well as a BMP1, TLL1 and/or TLL2 inhibitor which may be revealed (e.g., a compound disclosed in PCT application no. PCT/IB2015/050179 or PCT publication no. WO2015/104684) in the plasma samples are determined by LC-MS/MS. Concentrations at each time point are expressed as a percent of the initial test compound (e.g. prodrug) concentration in the incubation. The half-life for disappearance of the test compound is calculated from the slope of the log-transformed percent remaining versus time curve.

Generally, e.g., for screening purposes, a conversion of more than 10% of the test compound within 4 hours by this assay may be pursued as a pro-drug, although compounds having less conversion may also be useful as a pro-drug.

The compounds of Examples 1-14, 16-99, 102-103, 105-107, 109-111, 115, 117-141, 200-209, 300-301, 500, and 600-602, were tested in the human plasma stability assay and found to convert to a BMP1, TLL1 and/or TLL2 inhibitor within 24 hours:

The title compound of Examples 2, 3, 9, 10, 16, 31, 45-47, 50, 55-65, 68, 69, 73, 81, 82, 84, 88, 89, 92, 97, 109, 115, 141, 300, 301 revealed (3-ethoxy-5-(5-((((R)-2-((R)-1-(N-hydroxyformamido)propyl)heptanamido)methyl)carbamoyl) furan-2-yl)phenyl)phosphonic acid (title compound of Example 96 in PCT/IB2015/050179 and PCT publication no. WO2015/104684);

The title compound of Examples 1, 4, 5, 48, 49, 51, 54, 66, 67, 70-72, 74-80, 83, 85, 86, 91, 93, 94, 96, 98, 99, 106, 110, 111, 117-124, 126-131, 135, 201, 203-209, 500, 600-602 revealed (S)-2-(2-ethoxy-4-(5-((((R)-2-((R)-1-(N-hydroxyformamido)propyl)heptanamido)methyl) carbamoyl)furan-2-yl)benzamido)succinic acid (title compound of Example 74 in PCT/IB2015/050179 and PCT publication no. WO2015/104684);

The title compound of Examples 6-8, 11-14, 17-30, 32-44, 52, 53, 87, 90, 95 revealed (S)-2-(2-(carboxymethoxy)-4-(5-((((R)-2-((R)-1-(N-hydroxyformamido)propyl)heptanamido)methyl)carbamoyl)furan-2-yl)benzamido)succinic acid (title compound of Example 126 in PCT/IB2015/050179 and PCT publication no. WO2015/104684);

The title compound of Examples 136-140 and 200 revealed N—(((R)-2-((R)-1-(N-hydroxyformamido)propyl)heptanamido)methyl)-5-phenylfuran-2-carboxamide (title compound of Example 24 in PCT/IB2015/050179 and PCT publication no. WO2015/104684);

The title compound of Examples 102, 103, 105, 202 revealed 4-(5-((((R)-2-((R)-1-(N-hydroxyformamido) propyl)heptanamido)methyl)carbamoyl)furan-2-yl)benzoic acid (title compound of Example 71 in PCT/IB2015/050179 and PCT publication no. WO2015/104684);

The title compound of Examples 125, 132-134 revealed ((2-ethoxy-4-(5-((((R)-2-((R)-1-(N-hydroxyformamido)propyl)heptanamido)methyl)carbamoyl)furan-2-yl)benzamido)methyl)phosphonic acid (title compound of Example 139 in PCT/IB2015/050179 and PCT publication no. WO2015/104684);

The title compound of Examples 107 revealed (((3-ethoxy-5-(5-((((R)-2-((R)-1-(N-hydroxyformamido)propyl)heptanamido)methyl)carbamoyl)furan-2-yl)phenyl)(hydroxy)phosphoryl)oxy)methyl isopropyl carbonate (title compound of Example 302 in the current application).

At least the compounds of Examples 1-14 and 136-141 were tested in the human plasma stability assay and found to convert >10% (in 4 hours) to a BMP1, TLL1 and/or TLL2 inhibitor.

### Solution Stability Assay

A solution stability assay may be used to determine the ability of a compound of the invention to convert to a BMP1, TLL1 and/or TLL2 inhibitor derivative (for example, a compound disclosed in PCT application no. PCT/IB2015/050179 or PCT publication no. WO2015/104684), in solution.

A 1 mg/mL of the test compound in 100 mM Britton Robinson buffer pH 7.96 is placed in an amber vial and heated to 40° C. Aliquots are removed at 24 hr intervals and injected five times on LCMS. Samples are monitored at 300 nM and relative percent purity is determined by averaging the five injections at time=0 h and each subsequent time point up to 200 hr. The amount of the test compound present at each timepoint is determined by averaging the percent present from the five runs and is reported as percent present relative to time zero point which is 100%. A linear regression is performed and the relative % purity is determined at 200 hr.

LCMS Method: Shimadzu 10Avp with Sedere Sedex 75C and Waters ZQ Single Quadrupole using electrospray positive [ES+ve to give M+H⁺] equipped with a Thermo Hypersil Gold C18 1.9 μm column (2.1 mm×20 mm i.d.) eluting with 0.02% TFA in water (solvent A) and 0.02% TFA in CH₃CN (solvent B), using the following elution gradient: 4-95% (solvent B) over 1.88 min and holding at 4% for 0.9 min at a flow rate of 1.4 mL/min.

Generally, e.g., for screening purposes, a purity of less than 80% (reflecting at least 20% conversion of a test compound) at 200 hr may be pursued as a pre-drug, although compounds having less conversion may also be useful as a pre-drug.

The compounds of examples 1-9, 12-14, and 136-142 were tested in the solution stability assay and found to have <80% purity present at 200 hours.

1. A compound of Formula (I): 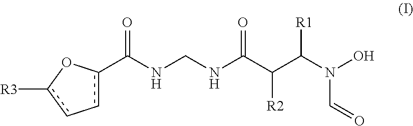
or a pharmaceutically acceptable salt thereof,
wherein:
R1 is selected from the group consisting of H, (C₁-C₄) straight chain alkyl, and (C₁-C₄) straight chain alkyl substituted with one hydroxy group;
R2 is selected from H, (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl, wherein heterocyclyl is a monocyclic ring having 5-6 ring atoms wherein 1-2 of the ring atoms are selected from nitrogen, oxygen and sulfur, and wherein said (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl are optionally substituted with 1-2 groups independently selected from (C₁-C₄)alkyl, (C₁-C₄)alkoxy, halo, and cyano; and
R3 is selected from phenyl and heteroaryl, wherein said phenyl and heteroaryl are substituted with 1-3 substituents independently selected from:
(C₁-C₆)alkyl, optionally substituted with 1-3 groups independently selected from: fluoro; —CO₂R′; —P(O)R″R″; —NR^{a}R^{b} wherein R^{a} is selected from H and (C₁-C₄)alkyl and R^{b} is selected from (C₁-C₄)alkyl substituted with 1-3 groups independently selected from —CO₂R′ and —P(O)R″R″; and —C(O)NR^{a1}R^{b1} wherein R^{a1} and R^{b1} are independently selected from H and (C₁-C₄)alkyl, wherein the (C₁-C₄)alkyl is optionally substituted with 1-3 groups independently selected from hydroxy, —CO₂R′, -and —P(O)R″R″;
cyclopropyl, optionally substituted with one —CO₂R′;
—C(O)NR^{a2}R^{b2} wherein R^{a2} and R^{b2} are independently selected from H and (C₁-C₄)alkyl, wherein the (C₁-C₄)alkyl is optionally substituted with 1-3 groups independently selected from hydroxy, —CO₂R′, —P(O)R″R″, —NR^{c}R^{d} and —N⁺R^{c}R^{d}R^{e};
(C₁-C₆)alkoxy, optionally substituted with 1-3 substituents independently selected from halo, hydroxy, —CO₂R′, (C₃-C₆)cycloalkyl, —C(O)NH₂ and pyrrolidinyl;
(C₃-C₆)cycloalkoxy, optionally substituted with 1-3 substituents independently selected from halo, hydroxy, and —CO₂R′;
—NR^{a3}R^{b3} wherein R^{a3} and R^{b3} are independently selected from H and (C₁-C₄)alkyl, wherein the (C₁-C₄)alkyl is optionally substituted with 1-3 groups independently selected from oxo and —CO₂R′;
—SR^{a4} wherein R^{a4} is selected from H and (C₁-C₄)alkyl;
—CO₂R′; —C(NOH)NH₂; cyano; —P(O)R″R″; —OP(O)R″R″; halo; hydroxy; nitro; —NHSO₂(C₁-C₂)alkyl; —SO₃H; —SO₂(C₁-C₂)alkyl; —SO₂NR^{c}R^{d}; —SO₂NHC(O)(C₁-C₂)alkyl; and —B(OH)₂;
wherein in each occurrence:
R^{c}, R^{d} and R^{e} are independently selected from H and (C₁-C₂)alkyl;
R′ is independently selected from H, phenyl, (5-methyl-2-oxo-1,3-dioxol-4-yl)methyl: 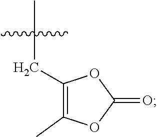 [SMILES: *Cc1oc(=O)oc1C]
and (C₁-C₄)alkyl, wherein said (C₁-C₄)alkyl is optionally substituted with 1-2 groups independently selected from phenyl, heteroaryl, —NR^{f}R^{f} wherein each R^{f} is independently selected from H and (C₁—C₄)alkyl, heterocycloalkyl optionally substituted with one oxo, —OC(O)O(C₁-C₄)alkyl, —OC(O)R^{g} wherein R^{g} is (C₁-C₄)alkyl or phenyl, and —C(O)NR^{h}R^{h} wherein R^{h} is independently selected from H and (C₁-C₄)alkyl;
R″ is independently selected from the group consisting of: —OH; (C₁-C₄)alkyl; (C₁-C₄)alkoxy; benzyloxy; phenoxy; (5-methyl-2-oxo-1,3-dioxol-4-yl)methoxy: 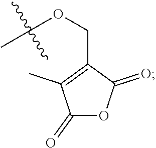 [SMILES: *OCC1=C(C)C(=O)OC1=O]
and —OCR^{a5}R^{b5}OC(O)R^{z}, wherein: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl;
provided that at least one of said 1-3 phenyl and heteroaryl substituents comprises at least 1 group selected from:
—CO₂R′, wherein R′ is phenyl, (5-methyl-2-oxo-1,3-dioxol-4-yl)methyl, or (C₁-C₄)alkyl, wherein said (C₁-C₄)alkyl is optionally substituted with 1-2 groups independently selected from phenyl, heteroaryl, NR^{f}R^{f} wherein each R^{f} is independently selected from H and (C₁-C₄)alkyl, heterocycloalkyl, —OC(O)O(C₁-C₄)alkyl, —OC(O)R^{g} wherein R^{g} is (C₁-C₄)alkyl or phenyl, and —C(O)NR^{h}R^{h} wherein R^{h} is independently selected from H and (C₁-C₄)alkyl;
—P(O)R″R″, wherein one R″ is selected from: (C₁-C₄)alkoxy; benzyloxy; phenoxy; (5-methyl-2-oxo-1,3-dioxol-4-yl)methoxy; and —OCR^{a5}R^{b5}OC(O)R^{z}, and the other R″ is selected from: OH; (C₁-C₄)alkyl; (C₁-C₄)alkoxy; benzyloxy; phenoxy; (5-methyl-2-oxo-1,3-dioxol-4-yl)methoxy; and —OCR^{a5}R^{b5}OC(O)R^{z} wherein in each occurrence: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl; and
—OP(O)R″R″, wherein one R″ is selected from: (C₁-C₄)alkoxy; benzyloxy; phenoxy; (5-methyl-2-oxo-1,3-dioxol-4-yl)methoxy; and —OCR^{a5}R^{b5}OC(O)R^{z}, and the other R″ is selected from: OH; (C₁-C₄)alkyl; (C₁-C₄)alkoxy; benzyloxy; phenoxy; (5-methyl-2-oxo-1,3-dioxol-4-yl)methoxy; and —OCR^{a5}R^{b5}OC(O)R^{z} wherein in each occurrence: R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl).

2. The compound or salt thereof according to claim 1, wherein the compound according to Formula (I) has the Formula (I)(a):

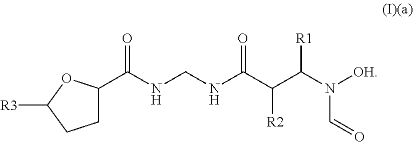

3. The compound or salt thereof according to claim 1, wherein the compound according to Formula (I) has the Formula (I)(b):

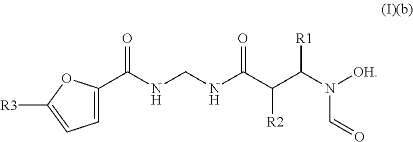

4. The compound or salt thereof according to claim 1, wherein R1 is H, methyl, ethyl or —CH₂OH.

5. The compound or salt thereof according to claim 1, wherein R2 is H or optionally substituted: n-pentyl, 2-ethylbutyl, (cyclopentyl)methyl, benzyl, 2-phenylethyl, 3-phenylpropyl, or 2-naphthylethyl.

6. The compound or salt thereof according to claim 1, wherein:
R1 is H, and R2 has (R) stereochemistry;
R1 is —CH₂OH and has (S) stereochemistry, and R2 has (R) stereochemistry; or
R1 is (C₁-C₄) straight chain alkyl or (C₂-C₄) straight chain alkyl substituted with one hydroxy group, and both R1 and R2 have (R) stereochemistry.

7. The compound or salt thereof according to claim 1, wherein R′ is independently selected from (C₁-C₄)alkyl and benzyl, and R″ is independently selected from the group consisting of: (C₁-C₄)alkyl; (C₁-C₄)alkoxy; benzyloxy; and —OCR^{a5}R^{b5}OC(O)R^{z}.

8. The compound or salt thereof according to claim 1 wherein at least one of said 1-3 substituents of R3 phenyl or heteroaryl comprises: at least one —CO₂R′ group, wherein R′ is (C₁-C₄)alkyl or benzyl: or at least one —P(O)R″R″ or —OP(O)R″R″ group, wherein one or both R″ of said P(O)R″R″ or —OP(O)R″R″ is independently C₃- or C₄-alkyl; or at least one —P(O)R″R″ or —OP(O)R″R″ group, wherein one or both R″ of said P(O)R″R″ or —OP(O)R″R″ is independently selected from said benzyloxy and —OCR^{a5}R^{b5}OC(O)R^{z}.

9-10. (canceled)

11. The compound or salt thereof according to claim 1, wherein R3 is substituted phenyl.

12-16. (canceled)

17. The compound or salt thereof according to claim 1, wherein R3 is substituted heteroaryl.

18. (canceled)

19. A compound or salt according to claim 1, wherein the compound of Formula (I) is selected from the group consisting of:
(S)-dimethyl 2-(4-(5-((((R)-2-((R)-1-(N-
hydroxyformamido)propyl)heptanamido)methyl)carbamoyl)furan-2-yl)-2-(2-methoxy-2-
oxoethoxy)benzamido)succinate
ethyl 4-(5-((((R)-2-((R)-1-(N-hydroxyformamido)propyl)heptanamido)methyl)carbamoyl)furan-
2-yl)benzoate
butyl 4-(5-((((R)-2-((R)-1-(N-hydroxyformamido)propyl)heptanamido)methyl)carbamoyl)furan-
2-yl)benzoate
phenethyl 4-(5-((((R)-2-((R)-1-(N-
hydroxyformamido)propyl)heptanamido)methyl)carbamoyl)furan-2-yl)benzoate
2-morpholinoethyl 4-(5-((((R)-2-((R)-1-(N-
hydroxyformamido)propyl)heptanamido)methyl)carbamoyl)furan-2-yl)benzoate
2-(dimethylamino)ethyl 4-(5-((((R)-2-((R)-1-(N-
hydroxyformamido)propyl)heptanamido)methyl)carbamoyl)furan-2-yl)benzoate
2-(dimethylamino)-2-oxoethyl 4-(5-((((R)-2-((R)-1-(N-
hydroxyformamido)propyl)heptanamido)methyl)carbamoyl)furan-2-yl)benzoate
(S)-3-(2-ethoxy-4-(5-((((R)-2-((R)-1-(N-
hydroxyformamido)propyl)heptanamido)methyl)carbamoyl)furan-2-yl)benzamido)-4-
((isobutyryloxy)methoxy)-4-oxobutanoic acid
(((3-ethoxy-5-(5-((((R)-2-((R)-1-(N-
hydroxyformamido)propyl)heptanamido)methyl)carbamoyl)furan-2-
yl)phenyl)phosphoryl)bis(oxy))bis(methylene) diisopropyl dicarbonate
diphenyl (3-ethoxy-5-(5-((((R)-2-((R)-1-(N-
hydroxyformamido)propyl)heptanamido)methyl)carbamoyl)furan-2-yl)phenyl)phosphonate
(((3-ethoxy-5-(5-((((R)-2-((R)-1-(N-
hydroxyformamido)propyl)heptanamido)methyl)carbamoyl)furan-2-
yl)phenyl)phosphoryl)bis(oxy))bis(methylene) bis(2-methylpropanoate)
(S)-3-(2-ethoxy-4-(5-((((R)-2-((R)-1-(N-
hydroxyformamido)propyl)heptanamido)methyl)carbamoyl)furan-2-yl)benzamido)-4-oxo-4-
phenoxybutanoic acid
(S)-bis((isobutyryloxy)methyl) 2-(2-ethoxy-4-(5-((((R)-2-((R)-1-(N-
hydroxyformamido)propyl)heptanamido)methyl)carbamoyl)furan-2-yl)benzamido)succinate
(S)-di-tert-butyl 2-(2-ethoxy-4-(5-((((R)-2-((R)-1-(N-
hydroxyformamido)propyl)heptanamido)methyl)carbamoyl)furan-2-yl)benzamido)succinate
(S)-4-ethoxy-3-(2-ethoxy-4-(5-((((R)-2-((R)-1-(N-
hydroxyformamido)propyl)heptanamido)methyl)carbamoyl)furan-2-yl)benzamido)-4-
oxobutanoic acid
(S)-4-(tert-butoxy)-3-(2-ethoxy-4-(5-((((R)-2-((R)-1-(N-
hydroxyformamido)propyl)heptanamido)methyl)carbamoyl)furan-2-yl)benzamido)-4-
oxobutanoic acid
(((3-ethoxy-5-(5-((((R)-2-((R)-1-(N-
hydroxyformamido)propyl)heptanamido)methyl)carbamoyl)furan-2-
yl)phenyl)phosphoryl)bis(oxy))bis(methylene) diacetate
(S)-4-((benzoyloxy)methoxy)-3-(2-ethoxy-4-(5-((((R)-2-((R)-1-(N-
hydroxyformamido)propyl)heptanamido)methyl)carbamoyl)furan-2-yl)benzamido)-4-
oxobutanoic acid
(S)-diethyl 2-(2-ethoxy-4-(5-((((R)-2-((R)-1-(N-
hydroxyformamido)propyl)heptanamido)methyl)carbamoyl)furan-2-yl)benzamido)succinate
(S)-3-(2-ethoxy-4-(5-((((R)-2-((R)-1-(N-
hydroxyformamido)propyl)heptanamido)methyl)carbamoyl)furan-2-yl)benzamido)-4-((5-
methyl-2-oxo-1,3-dioxol-4-yl)methoxy)-4-oxobutanoicacid
(((3-ethoxy-5-(5-((((R)-2-((R)-1-(N-
hydroxyformamido)propyl)heptanamido)methyl)carbamoyl)furan-2-
yl)phenyl)(hydroxy)phosphoryl)oxy)methyl isobutyrate
(5-methyl-2-oxo-1,3-dioxol-4-yl)methyl hydrogen (3-ethoxy-5-(5-((((R)-2-((R)-1-(N-
hydroxyformamido)propyl)heptanamido)methyl)carbamoyl)furan-2-yl)phenyl)phosphonate
(((3-ethoxy-5-(5-((((R)-2-((R)-1-(N-
hydroxyformamido)propyl)heptanamido)methyl)carbamoyl)furan-2-
yl)phenyl)(hydroxy)phosphoryl)oxy)methyl isopropyl carbonate; and
phenyl hydrogen (3-ethoxy-5-(5-((((R)-2-((R)-1-(N-
hydroxyformamido)propyl)heptanamido)methyl)carbamoyl)furan-2-yl)phenyl)phosphonate.

20. A compound of Formula (II): 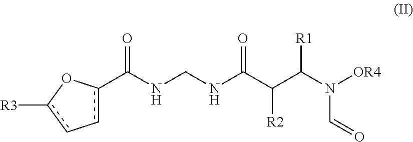
or a pharmaceutically acceptable salt thereof,
wherein:
R1 is selected from the group consisting of H, (C₁-C₄) straight chain alkyl, and (C₁-C₄) straight chain alkyl substituted with one hydroxy group;
R2 is selected from H, (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl, wherein heterocyclyl is a monocyclic ring having 5-6 ring atoms wherein 1-2 of the ring atoms are selected from nitrogen, oxygen and sulfur, and wherein said (C₁-C₁₁)alkyl, (C₁-C₃)alkyl-(C₃-C₆)cycloalkyl, (C₁-C₃)alkyl-phenyl, (C₁-C₃)alkyl-naphthyl and (C₁-C₃)alkyl-heterocyclyl are optionally substituted with 1-2 groups independently selected from (C₁-C₄)alkyl, (C₁-C₄)alkoxy, halo, and cyano; and
R3 is selected from: phenyl and heteroaryl, wherein said phenyl and heteroaryl are optionally substituted with 1-3 substituents independently selected from:
(C₁-C₆)alkyl, optionally substituted with 1-3 groups independently selected from: fluoro; —CO₂R′; —P(O)R″R″; —NR^{a}R^{b} wherein R^{a} is selected from H and (C₁-C₄)alkyl and R^{b} is selected from (C₁-C₄)alkyl substituted with 1-3 groups independently selected from —CO₂R′ and —P(O)R″R″; and —C(O)NR^{a1}R^{b1} wherein R^{a1} and R^{b1} are independently selected from H and (C₁-C₄)alkyl, wherein the (C₁-C₄)alkyl is optionally substituted with 1-3 groups independently selected from hydroxy, —CO₂R′, and —P(O)R″R″;
cyclopropyl, optionally substituted with one —CO₂R′;
—C(O)NR^{a2}R^{b2} wherein R^{a2} and R^{b2} are independently selected from H and (C₁-C₄)alkyl, wherein the (C₁-C₄)alkyl is optionally substituted with 1-3 groups independently selected from hydroxy, —CO₂R′, —P(O)R″R″, —NR^{c}R^{d} and —N⁺R^{c}R^{d}R^{e};
(C₁-C₆)alkoxy, optionally substituted with 1-3 substituents independently selected from halo, hydroxy, —CO₂R′, (C₃-C₆)cycloalkyl, —C(O)NH₂ and pyrrolidinyl;
(C₃-C₆)cycloalkoxy, optionally substituted with 1-3 substituents independently selected from halo, hydroxy, and —CO₂R′;
—NR^{a3}R^{b3} wherein R^{a3} and R^{b3} are independently selected from H and (C₁-C₄)alkyl, wherein the (C₁-C₄)alkyl is optionally substituted with 1-3 groups independently selected from oxo and —CO₂R′;
—SR^{a4} wherein R^{a4} is selected from H and (C₁-C₄)alkyl;
—CO₂R′; —C(NOH)NH₂; cyano; —P(O)R″R″; —OP(O)R″R″; halo; hydroxy; nitro; —NHSO₂(C₁-C₂)alkyl; —SO₃H; —SO₂(C₁-C₂)alkyl; —SO₂NR^{c}R^{d}; —SO₂NHC(O)(C₁-C₂)alkyl; and —B(OH)₂;
wherein in each occurrence:
R^{c}, R^{d} and R^{e} are independently selected from H and (C₁-C₂)alkyl;
R′ is independently selected from H, phenyl, (5-methyl-2-oxo-1,3-dioxol-4-yl)methyl, and (C₁-C₄)alkyl, wherein said (C₁-C₄)alkyl is optionally substituted with 1-2 groups independently selected from phenyl, heteroaryl, —NR^{f}R^{f} wherein each R^{f} is independently selected from H and (C₁-C₄)alkyl, heterocycloalkyl optionally substituted with one oxo, —OC(O)O(C₁-C₄)alkyl, —OC(O)R^{g} wherein R^{g} is (C₁-C₄)alkyl or phenyl, and —C(O)NR^{h}R^{h} wherein R^{h} is independently selected from H and (C₁-C₄)alkyl;
R″ is independently selected from the group consisting of —OH; (C₁-C₄)alkyl; (C₁-C₄)alkoxy; benzyloxy; phenoxy; (5-methyl-2-oxo-1,3-dioxol-4-yl)methoxy; and
—OCR^{a5}R^{b5}OC(O)R^{z} wherein R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl; and
R4 is selected from the groups A-G defined below:
A. —C(O)^{x} where R^{x} is selected from:
a. —NRⁱRⁱ where one Rⁱ is H or (C₁-C₄)alkyl, and the other is a —C₂H₄NH-tripeptide group; or where each Rⁱ is independently selected from H, phenyl, (C₁-C₄)alkyl optionally substituted with 1-2 hydroxy or phenyl groups, —C(O)(C₁-C₄)alkyl, —C(O)(C₁-C₈)alkoxy, and phenyl;
b. phenyl, optionally substituted with 1-2 groups independently selected from:
hydroxy;
halo;
(C₁-C₄)alkyl optionally substituted with one group NR^{v}R^{v} where each R^{v} is independently selected from H and (C₁-C₄)alkyl;
—CO₂H;
(C₁-C₄)alkoxy;
—PO₃H₂;
—OPO₃H₂;
—OC(O)(C₁-C₄)alkyl;
—NR^{j}R^{j} where each R^{j} is independently selected from H and (C₁-C₄)alkyl wherein the (C₁-C₄)alkyl is optionally substituted with 1-4 groups independently selected from —NHMe and —NMe₂;
—CF₃;
—OCF₃;
—NO₂;
—B(OH)₂;
—OC(O)NR^{k}R^{k} wherein each R^{k} is independently selected from H and (C₁-C₄)alkyl;
—(CH₂)₀₋₁ heterocycloalkyl containing 1-2 ring nitrogens and/or 1-2 ring oxygens, optionally substituted with one oxo group;
heteroaryl optionally substituted with a C-linked pyranose group: 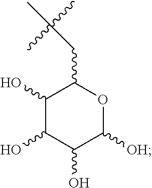 [SMILES: *CC1OC(O)C(O)C(O)C1O]
—O(C₁-C₄)alkyl substituted with one group selected from —NR^{l}R^{l} wherein each R^{l} is independently selected from H and (C₁-C₄)alkyl, heterocycloalkyl, heteroaryl wherein heteroaryl is optionally substituted with one C-linked pyranose group: 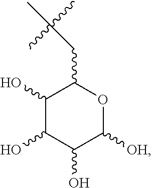 [SMILES: *CC1OC(O)C(O)C(O)C1O]
and —O(C₁-C₄)alkyl substituted with one —OH group;
—NR^{m}SO₂R^{m} wherein each R^{m} is independently selected from H and (C₁-C₄)alkyl;
—OCH₂OPO₃H₂;
an O-linked pyranose group selected from 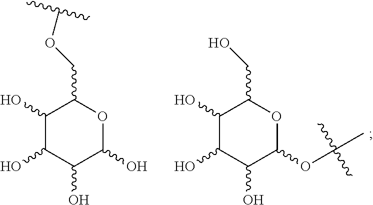 [SMILES: *OCC1OC(O)C(O)C(O)C1O ; *OC1OC(CO)C(O)C(O)C1O]
a group 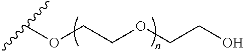
where n=an integer of from 1-20;
a group 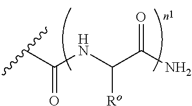
where n¹=an integer of from 1-5 and R^{o} is an amino acid side chain; and
a group 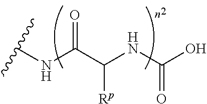
where n=an integer of from 1-5 and R^{p} is an amino acid side chain;
c. bicyclic heteroaryl containing a phenyl ring moiety fused to a heterocycloalkyl ring moiety, wherein the heterocycloalkyl ring has 1-4 ring atoms selected from nitrogen and oxygen and is optionally substituted with 1-2 (C₁-C₄)alkyl groups;
d. a group: 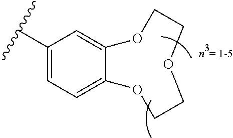
where n³ is an integer of from 1-5;
e. —NR^{q}C₂H₄OC(O)R^{r} where R^{q} is H or (C₁-C₄)alkyl and R^{r} is selected from: (C₁-C₅)alkyl; phenyl optionally substituted with 1-2 groups independently selected from hydroxy, halo, (C₁-C₄)alkyl, —CO₂H, (C₁-C₄)alkoxy, —PO₃H₂, and —OC(O)Me; pyridyl optionally substituted with 1-2 groups independently selected from hydroxy, halogen, (C₁-C₄)alkyl, —CO₂H, (C₁-C₄)alkoxy, —PO₃H₂, and —OC(O)Me; and (C₁-C₄) alkoxy;
f. (C₁-C₁₀)alkyl, optionally substituted with 1-2 phenyl groups;
g. (C₁-C₄)alkoxy;
h. monocyclic heteroaryl comprising 5 or 6 ring atoms comprising 1-3 heteroatoms selected from N and O, optionally substituted with 1-2 groups independently selected from hydroxy, halogen, (C₁-C₄)alkyl, —CO₂H, (C₁-C₄)alkoxy, —PO₃H₂, and —OC(O)Me;
i. naphthyl;
j. (C₁-C₄)alkyl optionally substituted with one —NR^{s}R^{s} where R^{s} is independently selected from: H, Me, and phenoxy optionally substituted with 1-2 groups independently selected from halogen, (C₁-C₄)alkyl, —CO₂H, (C₁-C₄)alkoxy, —PO₃H₂, and —OC(O)Me;
k. piperidinyl;
l. pyrrolidinyl; and
m. (C₃-C₆)cycloalkyl optionally substituted with one (C₁-C₄)alkyl group;
B. —CR^{a6}R^{b6}OR^{y} wherein each R^{a6} and R^{b6} is independently selected from H, (C₁-C₄)alkyl, phenyl, and (C₃-C₆)cycloalkyl, or R^{a6} and R^{b6} together with the carbon to which they are attached form a (C₃-C₆)cycloalkyl, and R^{y} is selected from:
a. —COR^{z1} where R^{z1} is selected from:
—NR^{t}R^{t} where one R^{t} is H or (C₁-C₄)alkyl, and the other is a —C₂H₄NH-tripeptide group;
phenyl optionally substituted with 1-2 groups independently selected from hydroxy, halo, (C₁-C₄)alkyl, —CO₂H, (C₁-C₄)alkoxy, —PO₃H₂, and —OC(O)Me;
benzyl;
(C₁-C₅)alkyl;
(C₁-C₄)alkoxy;
(C₁-C₄)alkyl optionally substituted with one —NR^{u}R^{u} where R^{u} is independently selected from H, Me, and phenoxy optionally substituted with 1-2 groups independently selected from halogen, (C₁-C₄)alkyl, —CO₂H, (C₁-C₄)alkoxy, —PO₃H₂, and —OC(O)Me;
pyridyl optionally substituted with 1-2 groups independently selected from hydroxy, halogen, (C₁-C₄)alkyl, —CO₂H, (C₁-C₄)alkoxy, —PO₃H₂, and —OC(O)Me;
pyrrolidinyl; and
—CH═CHCO₂H;
b. —P(O)R^{z2}R^{z2} where each R^{z2} is independently selected from:
H;
—OH;
(C₁-C₅)alkyl;
(C₁-C₅)alkoxy;
phenoxy optionally substituted with 1-2 groups independently selected from halo, —OMe, and —OEt;
benzyloxy;
—NR^{a7}R^{b7} wherein R^{a7} is selected from H and (C₁-C₄)alkyl and R^{b7} is selected from (C₁-C₄)alkyl substituted with 1-3 groups independently selected from: —CO₂R′^{A}, where R′^{A} is independently selected from H, (C₁-C₄)alkyl and benzyl; phenyl; —OH; —SH; —SMe; and phenyl substituted with one —OH, —NH₂, —C(O)NH₂, imidazol-4-yl, or indol-3-yl;
—NR^{a8}R^{b8} wherein R^{a8} and R^{b8} together with the nitrogen to which they are attached form a 4 to 6 membered heterocycloalkyl, optionally substituted with one —CO₂R′^{B} group, where R′^{B} is selected from H, (C₁-C₄)alkyl and benzyl;
—OCR^{a9}R^{b9}OC(O)R^{p1} wherein R^{a9} and R^{b9} are independently selected from H, (C₁-C₄)alkyl, phenyl, (C₁-C₄)alkoxy, and (C₃-C₆)cycloalkyl, or R^{a9} and R^{b9} together with the carbon to which they are attached form a (C₃-C₆)cycloalkyl; and R^{p1} is selected from (C₁-C₅)alkyl, (C₁-C₅)alkoxy, and phenyl;
c. a group which is: 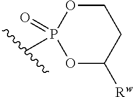
wherein R^{w} is selected from: phenyl optionally substituted with one halo, and pyridyl; and
d. a group selected from: 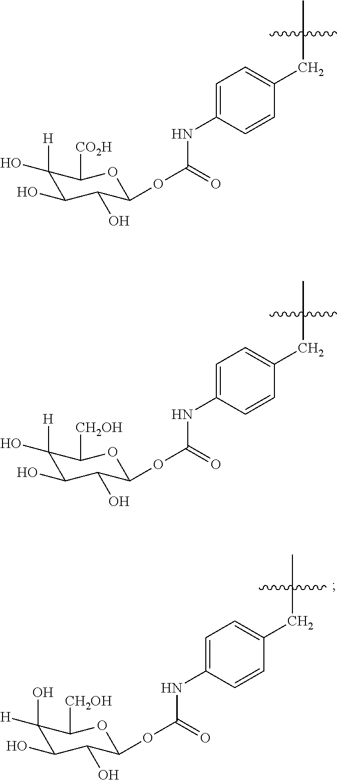 [SMILES: *Cc1ccc(NC(=O)OC2OC(C(=O)O)C(O)C(O)C2O)cc1 ; *Cc1ccc(NC(=O)OC2OC(CO)C(O)C(O)C2O)cc1 ; *Cc1ccc(NC(=O)OC2OC(CO)C(O)C(O)C2O)cc1]
and
C. (5-methyl-2-oxo-1,3-dioxol-4-yl)methyl: 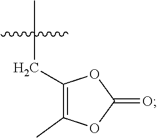 [SMILES: *Cc1oc(=O)oc1C]
D. —P(O)R^{z3}R^{z3} where each R^{z3} is independently selected from:
H;
—OH;
(C₁-C₅)alkyl;
(C₁-C₅)alkoxy;
phenoxy optionally substituted with 1-2 groups independently selected from halo, —OMe, and —OEt;
benzyloxy;
—NR^{a10}R^{b10} wherein R^{a10} is selected from H and (C₁-C₄)alkyl and R^{b10} is selected from (C₁-C₄)alkyl substituted with 1-3 groups independently selected from: —CO₂R′^{C}, where R′^{C} is independently selected from H, (C₁-C₄)alkyl and benzyl; phenyl; —OH; —SH; —SMe; and phenyl substituted with one group selected from —OH, —NH₂, —C(O)NH₂, imidazol-4-yl, and indol-3-yl;
—NR^{a11}R^{b11} wherein R^{a11} and R^{b11} together with the nitrogen to which they are attached form a 4 to 6 membered heterocycloalkyl optionally substituted with one —CO₂R′^{D} group, where R′^{D} is selected from H, (C₁-C₄)alkyl and benzyl; and
—OCR^{a12}R^{b12}OC(O)R^{p2} wherein R^{a12} and R^{b12} are independently selected from H, (C₁-C₄)alkyl, phenyl, (C₁-C₄)alkoxy, and (C₃-C₆)cycloalkyl, or R^{a12} and R^{b12} together with the carbon to which they are attached form a (C₃-C₆)cycloalkyl; and R^{p2} is selected from (C₁-C₅)alkyl, (C₁-C₅)alkoxy, and phenyl;
E. a group which is: 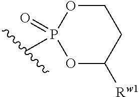
wherein R^{w1} is selected from: phenyl, phenyl substituted with one halo, and pyridyl;
F. a group selected from: 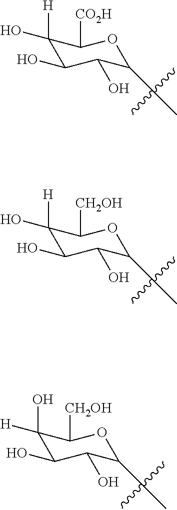 [SMILES: *C1OC(C(=O)O)C(O)C(O)C1O ; *C1OC(CO)C(O)C(O)C1O ; *C1OC(CO)C(O)C(O)C1O]
and
G. a group selected from:

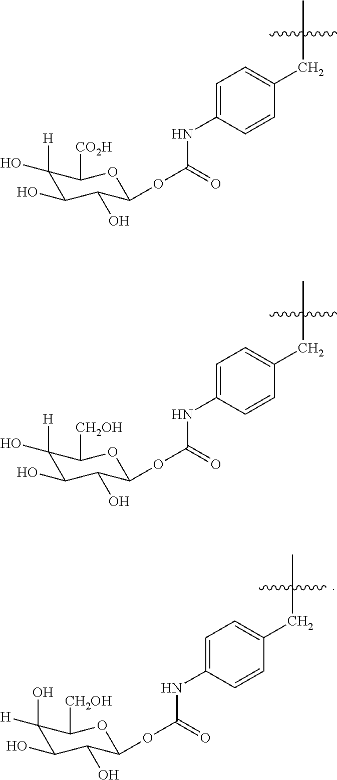 [SMILES: *Cc1ccc(NC(=O)OC2OC(C(=O)O)C(O)C(O)C2O)cc1 ; *Cc1ccc(NC(=O)OC2OC(CO)C(O)C(O)C2O)cc1 ; *Cc1ccc(NC(=O)OC2OC(CO)C(O)C(O)C2O)cc1]

21. The compound or salt thereof according to claim 20, wherein the compound according to Formula (II) has the Formula (II)(a):

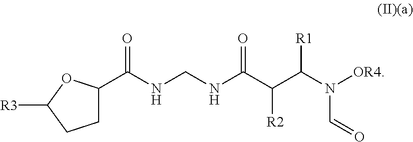

22. The compound or salt thereof according to claim 20, wherein the compound according to Formula (II) has the Formula (II)(b):

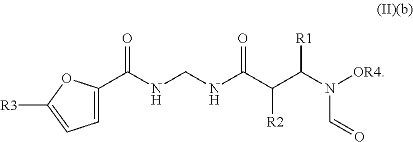

23. The compound or salt thereof according to claim 20, wherein R1 is H, methyl, ethyl or —CH₂OH.

24. The compound or salt thereof according to claim 20, wherein R2 is H or optionally substituted n-pentyl, 2-ethylbutyl, (cyclopentyl)methyl, benzyl, 2-phenylethyl, 3-phenylpropyl, or 2-naphthylethyl.

25. The compound or salt thereof according to claim 20, wherein:
R1 is H, and R2 has (R) stereochemistry; or
R1 is —CH₂OH and has (S) stereochemistry, and R2 has (R) stereochemistry; or
R1 is (C₁-C₄) straight chain alkyl, or (C₂-C₄) straight chain alkyl substituted with one hydroxy group, and both R1 and R2 have (R) stereochemistry.

26. The compound or salt thereof according to claim 20, wherein at least one of said R3 phenyl and heteroaryl substituents comprises at least 1 group selected from:
—CO₂R′, wherein R′ is (C₁-C₄)alkyl or benzyl;
—P(O)R″R″, wherein one R″ is selected from (C₁-C₄)alkoxy; benzyloxy; and —OCR^{a5}R^{b5}OC(O)R^{z} wherein: R^{a5} and R^{b5} are independently selected from H, (C₁-C₄)alkyl, phenyl, (C₁-C₄)alkoxy, and (C₃-C₆)cycloalkyl, or together with the carbon to which they are attached, form a (C₃-C₆)cycloalkyl; and R^{z} is selected from (C₁-C₅)alkyl, (C₁-C₅)alkoxy, and phenyl, and the other R″ is selected from the group defined for R″ according to Formula (II) (e.g., —OH; (C₁-C₄)alkyl; (C₁-C₄)alkoxy; benzyloxy; and —OCR^{a5}R^{b5}OC(O)R^{z} wherein R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl); and
—OP(O)R″R″, wherein one R″ is selected from (C₁-C₄)alkoxy; benzyloxy; and —OCR^{a5}R^{b5}OC(O)R^{z} wherein R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl, and the other R″ is selected from the group defined for R″ according to Formula (II) (e.g. —OH; (C₁-C₄)alkyl; (C₁-C₄)alkoxy; benzyloxy; and —OCR^{a5}R^{b5}OC(O)R^{z} wherein R^{a5} and R^{b5} are independently selected from H, (C₁-C₄)alkyl, phenyl, (C₁-C₄)alkoxy, and (C₃-C₆)cycloalkyl, or R^{a5} and R^{b5} together with the carbon to which they are attached form a (C₃-C₆)cycloalkyl; and R^{z} is selected from (C₁-C₅)alkyl, (C₁-C₅)alkoxy, and phenyl).

27. The compound or salt thereof according to claim 20, wherein R′ is H and R″ is independently selected from OH, (C₁-C₄)alkyl, and (C₁-C₄)alkoxy.

28. The compound or salt thereof according to claim 1, wherein R3 is selected from phenyl and substituted phenyl.

29-37. (canceled)

38. The compound or salt thereof according to claim 20, wherein R3 is optionally substituted heteroaryl.

39-41. (canceled)

42. The compound or salt thereof according to claim 20, wherein R4 is selected from: the groups A. —C(O)R^{x}; B. —CR^{a6}R^{b6}OR^{y}; C. (5-methyl-2-oxo-1,3-dioxol-4-yl)methyl; and D. —P(O)R^{z3}R^{z3} as defined above for Formula (II).

43. A compound or salt according to claim 20, wherein the compound of Formula (II) is selected from the group consisting of:
S)-dimethyl 2-(4-(5-((((R)-2-((R)-1-(N-
hydroxyformamido)propyl)heptanamido)methyl)carbamoyl)furan-2-yl)-2-(2-methoxy-2-
oxoethoxy)benzamido)succinate
ethyl 4-(5-((((R)-2-((R)-1-(N-hydroxyformamido)propyl)heptanamido)methyl)carbamoyl)furan-2-
yl)benzoate
butyl 4-(5-((((R)-2-((R)-1-(N-hydroxyformamido)propyl)heptanamido)methyl)carbamoyl)furan-2-
yl)benzoate
phenethyl 4-(5-((((R)-2-((R)-1-(N-
hydroxyformamido)propyl)heptanamido)methyl)carbamoyl)furan-2-yl)benzoate
2-morpholinoethyl 4-(5-((((R)-2-((R)-1-(N-
hydroxyformamido)propyl)heptanamido)methyl)carbamoyl)furan-2-yl)benzoate
2-(dimethylamino)ethyl 4-(5-((((R)-2-((R)-1-(N-
hydroxyformamido)propyl)heptanamido)methyl)carbamoyl)furan-2-yl)benzoate
2-(dimethylamino)-2-oxoethyl 4-(5-((((R)-2-((R)-1-(N-
hydroxyformamido)propyl)heptanamido)methyl)carbamoyl)furan-2-yl)benzoate
(S)-3-(2-ethoxy-4-(5-((((R)-2-((R)-1-(N-
hydroxyformamido)propyl)heptanamido)methyl)carbamoyl)furan-2-yl)benzamido)-4-
((isobutyryloxy)methoxy)-4-oxobutanoic acid
(((3-ethoxy-5-(5-((((R)-2-((R)-1-(N-
hydroxyformamido)propyl)heptanamido)methyl)carbamoyl)furan-2-
yl)phenyl)phosphoryl)bis(oxy))bis(methylene) diisopropyl dicarbonate
diphenyl (3-ethoxy-5-(5-((((R)-2-((R)-1-(N-
hydroxyformamido)propyl)heptanamido)methyl)carbamoyl)furan-2-yl)phenyl)phosphonate
(((3-ethoxy-5-(5-((((R)-2-((R)-1-(N-
hydroxyformamido)propyl)heptanamido)methyl)carbamoyl)furan-2-
yl)phenyl)phosphoryl)bis(oxy))bis(methylene) bis(2-methylpropanoate)
(S)-3-(2-ethoxy-4-(5-((((R)-2-((R)-1-(N-
hydroxyformamido)propyl)heptanamido)methyl)carbamoyl)furan-2-yl)benzamido)-4-oxo-4-
phenoxybutanoic acid
(S)-bis((isobutyryloxy)methyl) 2-(2-ethoxy-4-(5-((((R)-2-((R)-1-(N-
hydroxyformamido)propyl)heptanamido)methyl)carbamoyl)furan-2-yl)benzamido)succinate
(S)-di-tert-butyl 2-(2-ethoxy-4-(5-((((R)-2-((R)-1-(N-
hydroxyformamido)propyl)heptanamido)methyl)carbamoyl)furan-2-yl)benzamido)succinate
(S)-4-ethoxy-3-(2-ethoxy-4-(5-((((R)-2-((R)-1-(N-
hydroxyformamido)propyl)heptanamido)methyl)carbamoyl)furan-2-yl)benzamido)-4-oxobutanoic
acid
(S)-4-(tert-butoxy)-3-(2-ethoxy-4-(5-((((R)-2-((R)-1-(N-
hydroxyformamido)propyl)heptanamido)methyl)carbamoyl)furan-2-yl)benzamido)-4-oxobutanoic
acid
(((3-ethoxy-5-(5-((((R)-2-((R)-1-(N-
hydroxyformamido)propyl)heptanamido)methyl)carbamoyl)furan-2-
yl)phenyl)phosphoryl)bis(oxy))bis(methylene) diacetate
(S)-4-((benzoyloxy)methoxy)-3-(2-ethoxy-4-(5-((((R)-2-((R)-1-(N-
hydroxyformamido)propyl)heptanamido)methyl)carbamoyl)furan-2-yl)benzamido)-4-oxobutanoic
acid
(S)-diethyl 2-(2-ethoxy-4-(5-((((R)-2-((R)-1-(N-
hydroxyformamido)propyl)heptanamido)methyl)carbamoyl)furan-2-yl)benzamido)succinate
(S)-3-(2-ethoxy-4-(5-((((R)-2-((R)-1-(N-
hydroxyformamido)propyl)heptanamido)methyl)carbamoyl)furan-2-yl)benzamido)-4-((5-methyl-2-
oxo-1,3-dioxol-4-yl)methoxy)-4-oxobutanoicacid
(((3-ethoxy-5-(5-((((R)-2-((R)-1-(N-
hydroxyformamido)propyl)heptanamido)methyl)carbamoyl)furan-2-
yl)phenyl)(hydroxy)phosphoryl)oxy)methyl isobutyrate
(5-methyl-2-oxo-1,3-dioxol-4-yl)methyl hydrogen (3-ethoxy-5-(5-((((R)-2-((R)-1-(N-
hydroxyformamido)propyl)heptanamido)methyl)carbamoyl)furan-2-yl)phenyl)phosphonate
(((3-ethoxy-5-(5-((((R)-2-((R)-1-(N-
hydroxyformamido)propyl)heptanamido)methyl)carbamoyl)furan-2-
yl)phenyl)(hydroxy)phosphoryl)oxy)methyl isopropyl carbonate
phenyl hydrogen (3-ethoxy-5-(5-((((R)-2-((R)-1-(N-
hydroxyformamido)propyl)heptanamido)methyl)carbamoyl)furan-2-yl)phenyl)phosphonate
(S)-2-(4-(5-((((R)-2-((R)-1-(N-
(benzoyloxy)formamido)propyl)heptanamido)methyl)carbamoyl)furan-2-yl)-2-
ethoxybenzamido)succinic acid
(3-(5-((((R)-2-((R)-1-(N-(benzoyloxy)formamido)propyl)heptanamido)methyl)carbamoyl)furan-2-
yl)-5-ethoxyphenyl)phosphonic acid
(3-ethoxy-5-(5-((((R)-2-((R)-1-(N-(2-
phenylacetoxy)formamido)propyl)heptanamido)methyl)carbamoyl)furan-2-yl)phenyl)phosphonic
acid
(S)-2-(2-ethoxy-4-(5-((((R)-2-((R)-1-(N-(2-
phenylacetoxy)formamido)propyl)heptanamido)methyl)carbamoyl)furan-2-yl)benzamido)succinic
acid
(S)-2-(2-ethoxy-4-(5-((((R)-2-((R)-1-(N-
(pivaloyloxy)formamido)propyl)heptanamido)methyl)carbamoyl)furan-2-yl)benzamido)succinic
acid
(S)-2-(2-(carboxymethoxy)-4-(5-((((R)-2-((R)-1-(N-((2-
hydroxybenzoyl)oxy)formamido)propyl)heptanamido)methyl)carbamoyl)furan-2-
yl)benzamido)succinic acid
(S)-2-(4-(5-((((R)-2-((R)-1-(N-
(benzoyloxy)formamido)propyl)heptanamido)methyl)carbamoyl)furan-2-yl)-2-
(carboxymethoxy)benzamido)succinic acid
(S)-2-(2-(carboxymethoxy)-4-(5-((((R)-2-((R)-1-(N-((2-
methylbenzoyl)oxy)formamido)propyl)heptanamido)methyl)carbamoyl)furan-2-
yl)benzamido)succinic acid
(3-ethoxy-5-(5-(((4R,5R)-5-ethyl-6-formyl-11,11-dimethyl-3,10-dioxo-4-pentyl-7,9-dioxa-2,6-
diazadodecyl)carbamoyl)furan-2-yl)phenyl)phosphonic acid
(3-ethoxy-5-(5-((((R)-2-((R)-1-(N-((phosphonooxy)me-
thoxy)formamido)propyl)heptanamido)methyl)carbamoyl)furan-2-yl)phenyl)phosphonic acid
(S)-2-(2-(carboxymethoxy)-4-(5-((((R)-2-((R)-1-(N-
((phosphonooxy)methoxy)formamido)propyl)heptanamido)methyl)carbamoyl)furan-2-
yl)benzamido)succinic acid
(S)-2-(4-(5-((((R)-2-((R)-1-(N-((2-
acetoxybenzoyl)oxy)formamido)propyl)heptanamido)methyl)carbamoyl)furan-2-yl)-2-
(carboxymethoxy)benzamido)succinic acid
(S)-2-(2-(carboxymethoxy)-4-(5-((((R)-2-((R)-1-(N-
(nicotinoyloxy)formamido)propyl)heptanamido)methyl)carbamoyl)furan-2-yl)benzamido)succinic
acid
(S)-2-(2-(carboxymethoxy)-4-(5-(((4R,5R)-5-ethyl-6-formyl-10,10-dimethyl-3,8-dioxo-4-pentyl-
7,9-dioxa-2,6-diazaundecyl)carbamoyl)furan-2-yl)benzamido)succinic acid
5-phenyl-N-(((R)-2-((R)-1-(N-(pivaloyloxy)formamido)propyl)heptanamido)methyl)furan-2-
carboxamide
5-phenyl-N-(((R)-2-((R)-1-(N-(2-phenylacetoxy)formamido)propyl)heptanamido)methyl)furan-2-
carboxamide
N-((6R,7R)-6-ethyl-5-formyl-3,8-dioxo-7-pentyl-2,4-dioxa-5,9-diazadecan-10-yl)-5-phenylfuran-2-
carboxamide
N-((4R,5R)-5-ethyl-6-formyl-10,10-dimethyl-3,8-dioxo-4-pentyl-7,9-dioxa-2,6-diazaundecyl)-5-
phenylfuran-2-carboxamide
N-(((R)-2-((R)-1-(N-(benzoyloxy)formamido)propyl)heptanamido)methyl)-5-phenylfuran-2-
carboxamide
(3-ethoxy-5-(5-((((R)-2-((R)-1-(N-((5-methyl-2-oxo-1,3-dioxol-4-
yl)methoxy)formamido)propyl)heptanamido)methyl)carbamoyl)furan-2-yl)phenyl)phosphonic acid
(3-ethoxy-5-(5-((((R)-2-((R)-1-(N-((2-
hydroxybenzoyl)oxy)formamido)propyl)heptanamido)methyl)carbamoyl)furan-2-
yl)phenyl)phosphonic acid
(S)-2-(4-(5-((((R)-2-((R)-1-(N-((3-
carboxybenzoyl)oxy)formamido)propyl)heptanamido)methyl)carbamoyl)furan-2-yl)-2-
(carboxymethoxy)benzamido)succinic acid
(S)-2-(2-(carboxymethoxy)-4-(5-((((R)-2-((R)-1-(N-((1-
methylcyclopropanecarbonyl)oxy)formamido)propyl)heptanamido)methyl)carbamoyl)furan-2-
yl)benzamido)succinic acid
(2S)-2-(2-(carboxymethoxy)-4-(5-((((2R)-2-((1R)-1-(N-((2-methylcyclopropanecarbonyl)oxy)
formamido)propyl)heptanamido)methyl)carbamoyl)furan-2-yl)benzamido)succinic acid
(S)-2-(2-(carboxymethoxy)-4-(5-((((R)-2-((R)-1-(N-((4-
methoxybenzoyl)oxy)formamido)propyl)heptanamido)methyl)carbamoyl)furan-2-
yl)benzamido)succinic acid
(S)-2-(2-(carboxymethoxy)-4-(5-(((6R,7R)-6-ethyl-5-formyl-3,8-dioxo-7-pentyl-4-oxa-2,5,9-
triazadecan-10-yl)carbamoyl)furan-2-yl)benzamido)succinic acid
(S)-2-(4-(5-((((R)-2-((R)-1-(N-((2-
aminobenzoyl)oxy)formamido)propyl)heptanamido)methyl)carbamoyl)furan-2-yl)-2-
(carboxymethoxy)benzamido)succinic acid
(S)-2-(2-(carboxymethoxy)-4-(5-((((R)-2-((R)-1-(N-((2-
(methylamino)benzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-
ethylbutanoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((3,5-dimethylisoxazole-4-
carbonyl)oxy)formamido)propyl)heptanamido)methyl)carbamoyl)furan-2-yl)benzamido)succinic
acid
(S)-2-(2-(carboxymethoxy)-4-(5-((((R)-2-((R)-1-(N-((2,4-
dimethylnicotinoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-(2,2-
diphenylacetoxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-
isopropylbenzoyl)oxy)formamido)propyl)heptanamido)methyl)carbamoyl)furan-2-
yl)benzamido)succinic acid
(S)-2-(2-(carboxymethoxy)-4-(5-(((4R,5R)-5-ethyl-6-formyl-10,10-dimethyl-3,8-dioxo-4-pentyl-7-
oxa-2,6,9-triazaundecyl)carbamoyl)furan-2-yl)benzamido)succinic acid
(S)-2-(2-(carboxymethoxy)-4-(5-(((4R,5R)-5-ethyl-6-formyl-10-methyl-3,8-dioxo-4-pentyl-7-oxa-
2,6,9-triazaundecyl)carbamoyl)furan-2-yl)benzamido)succinic acid
(3-(5-((((R)-2-((R)-1-(N-((2-
((dimethylcarbamoyl)oxy)benzoyl)oxy)formamido)propyl)heptanamido)methyl)carbamoyl)furan-
2-yl)-5-ethoxyphenyl)phosphonic acid
(S)-2-(2-(carboxymethoxy)-4-(5-((((R)-2-((R)-1-(N-((2-
((dimethylcarbamoyl)oxy)benzoyl)oxy)formamido)propyl)heptanamido)methyl)carbamoyl)furan-
2-yl)benzamido)succinic acid
(S)-2-(4-(5-((((R)-2-((R)-1-(N-((1-
naphthoyl)oxy)formamido)propyl)heptanamido)methyl)carbamoyl)furan-2-yl)-2-
(carboxymethoxy)benzamido)succinic acid
(S)-2-(4-(5-((((R)-2-((R)-1-(N-((2-
naphthoyl)oxy)formamido)propyl)heptanamido)methyl)carbamoyl)furan-2-yl)-2-
(carboxymethoxy)benzamido)succinic acid
(S)-2-(2-(carboxymethoxy)-4-(5-((((R)-2-((R)-1-(N-((4-
(morpholinomethyl)benzoyl)oxy)formamido)propyl)heptanamido)methyl)carbamoyl)furan-2-
yl)benzamido)succinic acid
(S)-2-(2-(carboxymethoxy)-4-(5-(((4R,5R)-5-ethyl-6-formyl-10-methyl-3,8-dioxo-4-pentyl-10-
phenyl-7-oxa-2,6,9-triazaundecyl)carbamoyl)furan-2-yl)benzamido)succinic acid
(2S)-2-(2-(carboxymethoxy)-4-(5-((((2R)-2-((1R)-1-(N-(((((S)-1-methoxy-1-oxopropan-2-
yl)amino)(phenoxy)phosphoryl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-fluoro-6-
methylbenzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((4-methoxy-2-
methylbenzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((3-
methylisonicotinoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-methyl-4-
(morpholinomethyl)benzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((3,4-
dimethoxybenzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-
propylpentanoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2,4-
dimethoxybenzoyl)oxy)formamido)propyl)heptanamido)methyl)carbamoyl)furan-2-
yl)benzamido)succinic acid
(3-ethoxy-5-(5-((((R)-2-((R)-1-(N-((2-
ethylbutanoyl)oxy)formamido)propyl)heptanamido)methyl)carbamoyl)furan-2-
yl)phenyl)phosphonic acid
(3-ethoxy-5-(5-((((R)-2-((R)-1-(N-((2-fluoro-6-
methylbenzoyl)oxy)formamido)propyl)heptanamido)methyl)carbamoyl)furan-2-
yl)phenyl)phosphonic acid
(3-ethoxy-5-(5-((((R)-2-((R)-1-(N-((4-
methoxybenzoyl)oxy)formamido)propyl)heptanamido)methyl)carbamoyl)furan-2-
yl)phenyl)phosphonic acid
(S)-2-(2-ethoxy-4-(5-((((R)-2-((R)-1-(N-((2-
ethylbutanoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((2-fluoro-6-
methylbenzoyl)oxy)formamido)propyl)heptanamido)methyl)carbamoyl)furan-2-
yl)benzamido)succinic acid
(3-ethoxy-5-(5-((((R)-2-((R)-1-(N-((4-(pyrrolidin-1-
yl)benzoyl)oxy)formamido)propyl)heptanamido)methyl)carbamoyl)furan-2-yl)phenyl)phosphonic
acid
(S)-2-(2-ethoxy-4-(5-((((R)-2-((R)-1-(N-((4-
methoxybenzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((2-isopropyl-4-
methoxybenzoyl)oxy)formamido)propyl)heptanamido)methyl)carbamoyl)furan-2-
yl)benzamido)succinic acid
(S)-2-(2-(carboxymethoxy)-4-(5-((((R)-2-((R)-1-(N-((4-fluoro-2-
isopropylbenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-methoxy-2-
methylbenzoyl)oxy)formamido)propyl)heptanamido)methyl)carbamoyl)furan-2-
yl)benzamido)succinic acid
(3-ethoxy-5-(5-(((4R,5R)-5-ethyl-6-formyl-10,10-dimethyl-3,8-dioxo-4-pentyl-7-oxa-2,6,9-
triazaundecyl)carbamoyl)furan-2-yl)phenyl)phosphonic acid
(3-ethoxy-5-(5-(((4R,5R)-5-ethyl-6-formyl-10-methyl-3,8-dioxo-4-pentyl-10-phenyl-7-oxa-2,6,9-
triazaundecyl)carbamoyl)furan-2-yl)phenyl)phosphonic acid
(3-ethoxy-5-(5-((((R)-2-((R)-1-(N-((4-
morpholinobenzoyl)oxy)formamido)propyl)heptanamido)methyl)carbamoyl)furan-2-
yl)phenyl)phosphonic acid
(3-(5-((((R)-2-((R)-1-(N-((4-(1H-pyrazol-1-
yl)benzoyl)oxy)formamido)propyl)heptanamido)methyl)carbamoyl)furan-2-yl)-5-
ethoxyphenyl)phosphonic acid
(3-ethoxy-5-(5-((((R)-2-((R)-1-(N-((4-(2-oxopyrrolidin-1-
yl)benzoyl)oxy)formamido)propyl)heptanamido)methyl)carbamoyl)furan-2-yl)phenyl)phosphonic
acid
(3-ethoxy-5-(5-((((R)-2-((R)-1-(N-((4-methyl-3,4-dihydro-2H-benzo[b][1,4]oxazine-7-
carbonyl)oxy)formamido)propyl)heptanamido)methyl)carbamoyl)furan-2-yl)phenyl)phosphonic
acid
(3-ethoxy-5-(5-((((R)-2-((R)-1-(N-((2-methyl-6-
((phosphonooxy)methoxy)benzoyl)oxy)formamido)propyl)heptanamido)methyl)carbamoyl)furan-
2-yl)phenyl)phosphonic acid
(3-ethoxy-5-(5-((((R)-2-((R)-1-(N-((3-fluoro-4-(pyrrolidin-1-
yl)benzoyl)oxy)formamido)propyl)heptanamido)methyl)carbamoyl)furan-2-yl)phenyl)phosphonic
acid
(3-ethoxy-5-(5-((((R)-2-((R)-1-(N-((3-(pyrrolidin-1-
yl)benzoyl)oxy)formamido)propyl)heptanamido)methyl)carbamoyl)furan-2-yl)phenyl)phosphonic
acid
(3-ethoxy-5-(5-((((R)-2-((R)-1-(N-((2-methyl-4-
(morpholinomethyl)benzoyl)oxy)formamido)propyl)heptanamido)methyl)carbamoyl)furan-2-
yl)phenyl)phosphonic acid
(3-ethoxy-5-(5-(((4R,5R)-5-ethyl-6-formyl-10-methyl-3,8-dioxo-4-pentyl-7-oxa-2,6,9-
triazaundecyl)carbamoyl)furan-2-yl)phenyl)phosphonic acid
(S)-2-(2-ethoxy-4-(5-((((R)-2-((R)-1-(N-((2-isopropyl-4-
methoxybenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-fluoro-2-
isopropylbenzoyl)oxy)formamido)propyl)heptanamido)methyl)carbamoyl)furan-2-
yl)benzamido)succinic acid
(3-ethoxy-5-(5-((((R)-2-((R)-1-(N-((4-fluoro-2-
isopropylbenzoyl)oxy)formamido)propyl)heptanamido)methyl)carbamoyl)furan-2-
yl)phenyl)phosphonic acid
(3-ethoxy-5-(5-((((R)-2-((R)-1-(N-
(nonanoyloxy)formamido)propyl)heptanamido)methyl)carbamoyl)furan-2-yl)phenyl)phosphonic
acid
(S)-2-(2-ethoxy-4-(5-((((R)-2-((R)-1-(N-((4-
(morpholinomethyl)benzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((2-methyl-4-
(morpholinomethyl)benzoyl)oxy)formamido)propyl)heptanamido)methyl)carbamoyl)furan-2-
yl)benzamido)succinic acid
(S)-2-(2-ethoxy-4-(5-(((4R,5R)-5-ethyl-6-formyl-10-methyl-3,8-dioxo-4-pentyl-7-oxa-2,6,9-
triazaundecyl)carbamoyl)furan-2-yl)benzamido)succinic acid
(3-ethoxy-5-(5-((((R)-2-((R)-1-(N-((4-
(morpholinomethyl)benzoyl)oxy)formamido)propyl)heptanamido)methyl)carbamoyl)furan-2-
yl)phenyl)phosphonic acid
(S)-2-(2-ethoxy-4-(5-(((4R,5R)-5-ethyl-6-formyl-10,10-dimethyl-3,8-dioxo-4-pentyl-7-oxa-2,6,9-
triazaundecyl)carbamoyl)furan-2-yl)benzamido)succinic acid
(S)-2-(2-ethoxy-4-(5-(((4R,5R)-5-ethyl-6-formyl-10-methyl-3,8-dioxo-4-pentyl-10-phenyl-7-oxa-
2,6,9-triazaundecyl)carbamoyl)furan-2-yl)benzamido)succinic acid
(S)-2-(2-ethoxy-4-(5-((((R)-2-((R)-1-(N-((3-(pyrrolidin-1-
yl)benzoyl)oxy)formamido)propyl)heptanamido)methyl)carbamoyl)furan-2-yl)benzamido)succinic
acid
(S)-2-(2-ethoxy-4-(5-((((R)-2-((R)-1-(N-((3-fluoro-4-(pyrrolidin-1-
yl)benzoyl)oxy)formamido)propyl)heptanamido)methyl)carbamoyl)furan-2-yl)benzamido)succinic
acid
(S)-2-(2-ethoxy-4-(5-((((R)-2-((R)-1-(N-((4-(2-oxopyrrolidin-1-
yl)benzoyl)oxy)formamido)propyl)heptanamido)methyl)carbamoyl)furan-2-yl)benzamido)succinic
acid
(S)-2-(4-(5-((((R)-2-((R)-1-(N-((4-(1H-pyrazol-1-
yl)benzoyl)oxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-
((phosphonooxy)methoxy)formamido)propyl)heptanamido)methyl)carbamoyl)furan-2-
yl)benzamido)succinic acid
(3-(5-((((R)-2-((R)-1-(N-((2,3-dihydrobenzo[b][1,4]dioxine-6-
carbonyl)oxy)formamido)propyl)heptanamido)methyl)carbamoyl)furan-2-yl)-5-
ethoxyphenyl)phosphonic acid
(3-ethoxy-5-(5-(((4R,5R)-5-ethyl-6-formyl-10,10-dimethyl-3,8-dioxo-4-pentyl-7,9-dioxa-2,6-
diazaundecyl)carbamoyl)furan-2-yl)phenyl)phosphonic acid
(S)-2-(4-(5-((((R)-2-((R)-1-(N-((2,3-dihydrobenzo[b][1,4]dioxine-6-
carbonyl)oxy)formamido)propyl)heptanamido)methyl)carbamoyl)furan-2-yl)-2-
ethoxybenzamido)succinic acid
(3-ethoxy-5-(5-((((R)-2-((R)-1-(N-((4-methyl-3,4-dihydro-2H-benzo[b][1,4]oxazine-6-
carbonyl)oxy)formamido)propyl)heptanamido)methyl)carbamoyl)furan-2-yl)phenyl)phosphonic
acid
(S)-2-(2-ethoxy-4-(5-((((R)-2-((R)-1-(N-((4-methyl-3,4-dihydro-2H-benzo[b][1,4]oxazine-6-
carbonyl)oxy)formamido)propyl)heptanamido)methyl)carbamoyl)furan-2-yl)benzamido)succinic
acid
(S)-2-(2-ethoxy-4-(5-(((4R,5R)-5-ethyl-6-formyl-10,10-dimethyl-3,8-dioxo-4-pentyl-7,9-dioxa-2,6-
diazaundecyl)carbamoyl)furan-2-yl)benzamido)succinic acid
(2S)-2-(2-(carboxymethoxy)-4-(5-((((R)-2-((R)-1-(N-((2-methoxy-6-
methylbenzoyl)oxy)formamido)propyl)heptanamido)methyl)carbamoyl)tetrahydrofuran-2-
yl)benzamido)succinic acid
(3-ethoxy-5-(5-((((R)-2-((R)-1-(N-((4-methoxy-2-
methylbenzoyl)oxy)formamido)propyl)heptanamido)methyl)carbamoyl)furan-2-
yl)phenyl)phosphonic acid
(3-ethoxy-5-(5-((((R)-2-((R)-1-(N-((2-isopropyl-4-
methoxybenzoyl)oxy)formamido)propyl)heptanamido)methyl)carbamoyl)furan-2-
yl)phenyl)phosphonic acid
(S)-2-(2-(carboxymethoxy)-4-(5-((((R)-2-((R)-1-(N-
((phenylcarbamoyl)oxy)formamido)propyl)heptanamido)methyl)carbamoyl)furan-2-
yl)benzamido)succinic acid
(S)-2-(4-(5-((((R)-2-((R)-1-(N-((3,4-
dimethoxybenzoyl)oxy)formamido)propyl)heptanamido)methyl)carbamoyl)furan-2-yl)-2-
ethoxybenzamido)succinic acid
(3-ethoxy-5-(5-((((R)-2-((R)-1-(N-
((phenylcarbamoyl)oxy)formamido)propyl)heptanamido)methyl)carbamoyl)furan-2-
yl)phenyl)phosphonic acid
(S)-2-(2-ethoxy-4-(5-((((R)-2-((R)-1-(N-((2-
methylbenzoyl)oxy)formamido)propyl)heptanamido)methyl)carbamoyl)furan-2-
yl)benzamido)succinic acid
(S)-2-(2-ethoxy-4-(5-(((4R,5R)-5-ethyl-6-formyl-11,11-dimethyl-3,10-dioxo-4-pentyl-7,9-dioxa-
2,6-diazadodecyl)carbamoyl)furan-2-yl)benzamido)succinic acid
N-{[2-(carboxymethoxy)-4-(5-{[(4R,5R)-5-ethyl-6-formyl-9,12,12-trimethyl-3,8,10-trioxo-4-
pentyl-7,11-dioxa-2,6,9-triazatridec-1-yl]carbamoyl}furan-2-yl)phenyl]carbonyl}-L-aspartic acid
(S)-2-(4-(5-((((R)-2-((R)-1-(N-((2,4-
dimethoxybenzoyl)oxy)formamido)propyl)heptanamido)methyl)carbamoyl)furan-2-yl)-2-
ethoxybenzamido)succinic acid
(3-ethoxy-5-(5-((((R)-2-((R)-1-(N-((2-methyl-4-
morpholinobenzoyl)oxy)formamido)propyl)heptanamido)methyl)carbamoyl)furan-2-
yl)phenyl)phosphonic acid
(S)-2-(2-ethoxy-4-(5-((((R)-2-((R)-1-(N-((2-
isopropylbenzoyl)oxy)formamido)propyl)heptanamido)methyl)carbamoyl)furan-2-
yl)benzamido)succinic acid
(S)-2-(4-(5-((((R)-2-((R)-1-(N-((2,4-
dimethylnicotinoyl)oxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-((4-fluoro-2-
methylbenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((piperidine-4-
carbonyl)oxy)formamido)propyl)heptanamido)methyl)carbamoyl)furan-2-yl)benzamido)succinic
acid
(S)-2-(2-ethoxy-4-(5-((((R)-2-((R)-1-(N-((4-
(trifluoromethyl)benzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-
nitrobenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-
hydroxybenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((2-fluoro-4-
methoxybenzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-methoxy-2-
(trifluoromethyl)benzoyl)oxy)formamido)propyl)heptanamido)methyl)carbamoyl)furan-2-
yl)benzamido)succinic acid
(S)-2-(2-ethoxy-4-(5-((((R)-2-((R)-1-(N-((4-methoxy-2-
(trifluoromethoxy)benzoyl)oxy)formamido)propyl)heptanamido)methyl)carbamoyl)furan-2-
yl)benzamido)succinic acid
((4-(5-((((R)-2-((R)-1-(N-(benzoyloxy)formamido)propyl)heptanamido)methyl)carbamoyl)furan-2-
yl)-2-ethoxybenzamido)methyl)phosphonic acid
(S)-2-(2-ethoxy-4-(5-((((R)-2-((R)-1-(N-((2-methyl-4-
(trifluoromethyl)benzoyl)oxy)formamido)propyl)heptanamido)methyl)carbamoyl)furan-2-
yl)benzamido)succinic acid
(S)-2-(4-(5-((((R)-2-((R)-1-(N-((4-(4H-1,2,4-triazol-4-
yl)benzoyl)oxy)formamido)propyl)heptanamido)methyl)carbamoyl)furan-2-yl)-2-
ethoxybenzamido)succinic acid
(S)-2-(4-(5-((((R)-2-((R)-1-(N-((3-(1H-pyrrol-1-
yl)benzoyl)oxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-((4-(N-
methyhnethylsulfonamido)benzoyl)oxy)formamido)propyl)heptanamido)methyl)carbamoyl)furan-
2-yl)benzamido)succinic acid
(S)-2-(4-(5-((((R)-2-((R)-1-(N-((4-(1H-pyrazol-1-
yl)benzoyl)oxy)formamido)propyl)heptanamido)methyl)carbamoyl)furan-2-yl)-2-
ethoxybenzamido)succinic acid
(S)-2-(4-(5-((((R)-2-((R)-1-(N-((4-(1H-imidazol-1-
yl)benzoyl)oxy)formamido)propyl)heptanamido)methyl)carbamoyl)furan-2-yl)-2-
ethoxybenzamido)succinic acid
((2-ethoxy-4-(5-((((R)-2-((R)-1-(N-((4-
methoxybenzoyl)oxy)formamido)propyl)heptanamido)methyl)carbamoyl)furan-2-
yl)benzamido)methyl)phosphonic acid
((2-ethoxy-4-(5-((((R)-2-((R)-1-(N-((4-methoxy-2-
methylbenzoyl)oxy)formamido)propyl)heptanamido)methyl)carbamoyl)furan-2-
yl)benzamido)methyl)phosphonic acid
((2-ethoxy-4-(5-((((R)-2-((R)-1-(N-((2-methyl-4-
(morpholinomethyl)benzoyl)oxy)formamido)propyl)heptanamido)methyl)carbamoyl)furan-2-
yl)benzamido)methyl)phosphonic acid
(S)-2-(2-ethoxy-4-(5-((((R)-2-((R)-1-(N-((4-(2-(2-
hydroxyethoxy)ethoxy)benzoyl)oxy)formamido)propyl)heptanamido)methyl)carbamoyl)furan-2-
yl)benzamido)succinic acid
(4R,5R)-4-ethyl-3-formyl-6,10-dioxo-5-pentyl-10-(5-phenylfuran-2-yl)-2-oxa-3,7,9-triazadecyl
dihydrogen phosphate
(S)-2-(2-ethoxy-4-(5-((((R)-2-((R)-1-(N-((2-methyl-6-
(phosphonooxy)benzoyl)oxy)formamido)propyl)heptanamido)methyl)carbamoyl)furan-2-
yl)benzamido)succinic acid
4-(5-((((R)-2-((R)-1-(N-(benzoyloxy)formamido)propyl)heptanamido)methyl)carbamoyl)furan-2-
yl)benzoic acid
(S)-2-(2-ethoxy-4-(5-((((R)-2-((R)-1-(N-
((phenylcarbamoyl)oxy)formamido)propyl)heptanamido)methyl)carbamoyl)furan-2-
yl)benzamido)succinic acid
(S)-2-(4-(5-((((R)-2-((R)-1-(N-((2-bromo-4-
methoxybenzoyl)oxy)formamido)propyl)heptanamido)methyl)carbamoyl)furan-2-yl)-2-
ethoxybenzamido)succinic acid
(S)-2-(4-(5-((((R)-2-((R)-1-(N-((2-chloro-4-
methoxybenzoyl)oxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-((4-
(methylamino)benzoyl)oxy)formamido)propyl)heptanamido)methyl)carbamoyl)furan-2-
yl)benzamido)succinic acid
(S)-2-(4-(5-((((R)-2-((R)-1-(N-((4-
aminobenzoyl)oxy)formamido)propyl)heptanamido)methyl)carbamoyl)furan-2-yl)-2-
ethoxybenzamido)succinic acid
(S)-2-(4-(5-((((R)-2-((R)-1-(N-((4-
(aminomethyl)benzoyl)oxy)formamido)propyl)heptanamido)methyl)carbamoyl)furan-2-yl)-2-
ethoxybenzamido)succinic acid
(S)-2-(2-ethoxy-4-(5-((((R)-2-((R)-1-(N-((2-methyl-4-
nitrobenzoyl)oxy)formamido)propyl)heptanamido)methyl)carbamoyl)furan-2-
yl)benzamido)succinic acid
(S)-2-(4-(5-((((R)-2-((R)-1-(N-((3-
aminobenzoyl)oxy)formamido)propyl)heptanamido)methyl)carbamoyl)furan-2-yl)-2-
ethoxybenzamido)succinic acid
(S)-2-(4-(5-((((R)-2-((R)-1-(N-((3-
(dimethylamino)benzoyl)oxy)formamido)propyl)heptanamido)methyl)carbamoyl)furan-2-yl)-2-
ethoxybenzamido)succinic acid; and
(S)-2-(2-ethoxy-4-(5-((((R)-2-((R)-1-(N-((3-
(methylamino)benzoyl)oxy)formamido)propyl)heptanamido)methyl)carbamoyl)furan-2-
yl)benzamido)succinic acid.

44. (canceled)

45. A pharmaceutical composition comprising the compound or pharmaceutically acceptable salt thereof according to claim 1, and one or more pharmaceutically acceptable excipients.

46. A method of treating a disease associated with BMP1, TLL1 and/or TLL2 activity in a human in need thereof comprising administering to said human a therapeutically effective amount of the compound or pharmaceutically acceptable salt thereof according to claim 1.

47-64. (canceled)

65. A pharmaceutical composition comprising the compound or pharmaceutically acceptable salt thereof according to claim 20, and one or more pharmaceutically acceptable excipients.

66. A method of treating a disease associated with BMP1, TLL1 and/or TLL2 activity in a human in need thereof comprising administering to said human a therapeutically effective amount of the compound or pharmaceutically acceptable salt thereof according to claim 20.
